(19) United States
(12) Patent Application Publication
(10) Pub. No.: US 2019/0216893 A1
(43) Pub. Date: Jul. 18, 2019

(54) COMPOSITIONS AND METHODS OF MODULATING IMMUNE RESPONSE

(71) Applicant: The Scripps Research Institute, La Jolla, CA (US)

(72) Inventor: Benjamin F. Cravatt, La Jolla, CA (US)

(21) Appl. No.: 16/306,362

(22) PCT Filed: Jun. 2, 2017
(86) PCT No.: PCT/US17/35748

§ 371 Date: Nov. 30, 2018

Related U.S. Application Data
(60) Provisional application No. 62/345,715, filed on Jun. 3, 2016.

(51) Int. Cl. A61K 38/17

(52) U.S. Cl. CPC A61K 38/1709

(57) ABSTRACT

Disclosed herein are methods, pharmaceutical compositions, and vaccines for modulating an immune response. Also disclosed, herein are methods, pharmaceutical compositions, and vaccines for inducing an immune response.

### CROSS-REFERENCE

This application claims the benefit of U.S. Provisional Application No. 62/345,715, filed on Jun. 3, 2016, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 31, 2017, is named 48054-705_601_SL.txt and is 3,856,317 bytes in size.

### BACKGROUND OF THE DISCLOSURE

The immune system is a complex network of responses and processes that protects an organism and enables the organism to fight against a foreign agent. In some instances, there are two types of immune response when presented with a foreign agent. In one instance, the immune system responds with a B cell-mediated response (e.g., humoral response or antibody-mediated response) when foreign agents (e.g., antigens and/or pathogens) are present in the lymph or blood. In another instance, the immune system responds with a T cell-mediated response (e.g., a cell-mediated response) when cells that display aberrant MHC markers are present. In some instances, both humoral response and cell-mediated response are triggered by a foreign agent when, e.g., both antigens and cells containing aberrant MHC markers are present.

### SUMMARY OF THE DISCLOSURE

In certain embodiments, disclosed herein include methods, pharmaceutical compositions, and vaccines for modulating an immune response. In some embodiments, included herein are methods of administering a small molecule fragment described herein for modulating an immune response. In additional embodiments, described herein are pharmaceutical compositions and vaccines which comprise a small molecule fragment described herein for modulating an immune response.

Disclosed herein, in certain embodiments, is a method of modulating an immune response in a subject in need thereof, comprising administering to the subject a therapeutically effective amount of a small molecule fragment of Formula (I):

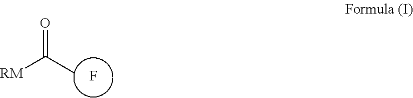

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with an endogenous cysteine-containing polypeptide expressed in the subject to form a cysteine-containing polypeptide-small molecule fragment adduct. In some embodiments, the small molecule fragment is covalently bound to a cysteine residue of the cysteine-containing polypeptide. In some embodiments, the cysteine-containing polypeptide-small molecule fragment adduct induces an immune response. In some embodiments, the cysteine-containing polypeptide-small molecule fragment adduct induces a humoral immune response. In some embodiments, the cysteine-containing polypeptide-small molecule fragment adduct induces a cell-mediated immune response. In some embodiments, the cysteine-containing polypeptide-small molecule fragment adduct increases an immune response relative to a control. In some embodiments, the cysteine-containing polypeptide-small molecule fragment adduct increases a humoral immune response relative to a control. In some embodiments, the cysteine-containing polypeptide-small molecule fragment adduct increases a cell-mediated immune response relative to a control. In some embodiments, the control is the level of an immune response in the subject prior to administration of the small molecule fragment. In some embodiments, the control is the level of an immune response in a subject who has not been exposed to the small molecule fragment. In some embodiments, the control is the level of a humoral immune response or a cell-mediated immune response in the subject prior to administration of the small molecule fragment. In some embodiments, the control is the level of a humoral immune response or a cell-mediated immune response in a subject who has not been exposed to the small molecule fragment. In some embodiments, the cysteine-containing polypeptide is overexpressed in a disease or condition. In some embodiments, the cysteine-containing polypeptide comprises one or more mutations. In some embodiments, the cysteine-containing polypeptide comprising one or more mutations is overexpressed in a disease or condition. In some embodiments, the disease or condition is cancer. In some embodiments, the cysteine-containing polypeptide is a cancer-associated protein. In some embodiments, the cysteine-containing polypeptide is overexpressed in a cancer. In some embodiments, the cysteine-containing polypeptide comprising one or more mutations is overexpressed in a cancer. In some embodiments, the cysteine-containing polypeptide is a non-denatured form of the polypeptide. In some embodiments, the cysteine-containing polypeptide comprises a biologically active cysteine site. In some embodiments, the biologically active cysteine site is a cysteine residue that is located about 10 Å or less to an active-site ligand or residue. In some embodiments, the cysteine residue that is located about 10 Å or less to the active-site ligand or residue is an active site cysteine. In some embodiments, the biologically active cysteine site is an active site cysteine. In some embodiments, the biologically active cysteine site is a cysteine residue that is located greater than 10 Å from an active-site ligand or residue. In some embodiments, the cysteine residue that is located greater than 10 Å from the active-site ligand or residue is a non-active site cysteine. In some embodiments, the biologically active cysteine site is a non-active site cysteine. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide is an enzyme, a transporter, a receptor, a channel protein, an adaptor protein, a chaperone, a signaling protein, transcription related protein, or translation related protein. In some embodiments, the enzyme comprises kinases, proteases, or deubiquitinating enzymes. In some embodiments, the protease is a cysteine protease. In some embodiments, the cysteine protease comprises caspases. In some embodiments, the signaling protein comprises vascular endothelial growth factor. In some embodiments, the signaling protein comprises a redox signaling protein. In some embodiments, the cysteine-containing polypeptide is about 20, 30, 35, 40, 45, 50, 55, 60, 65, 70, 75, 80, 85, 90, 95, 100 amino acid residues in length or more. In some embodiments, the cysteine-containing polypeptide comprises a protein illustrated in Tables 1-5. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in FIG. 1 (e.g., FIG. 1A and/or FIG. 1B). In some embodiments, F further comprises a linker moiety that connects F to the carbonyl moiety. In some embodiments, the small molecule fragment is a small molecule fragment illustrated in FIG. 1 (e.g., FIG. 1A and/or FIG. 1B). In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the method further comprises administering a cysteine-containing polypeptide-small molecule fragment adduct. In some embodiments, the cysteine-containing polypeptide is at most 50 amino acid residues in length. In some embodiments, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQ ID NOs: 1-9655. In some embodiments, the method further comprises administration of an adjuvant. In some embodiments, the small molecule fragment is formulated for parenteral, oral, or intranasal administration. In some embodiments, the subject is a human.

Disclosed herein, in certain embodiments, is a vaccine comprising a small molecule fragment of Formula (I):

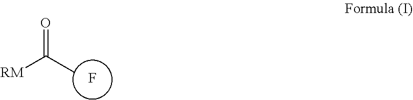

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with a cysteine-containing polypeptide to form a cysteine-containing polypeptide-small molecule fragment adduct. In some embodiments, the small molecule fragment is covalently bond to a cysteine residue of the cysteine-containing polypeptide. In some embodiments, the cysteine-containing polypeptide is an endogenous cysteine-containing polypeptide expressed in a subject. In some embodiments, administration of the small molecule fragment induces an immune response. In some embodiments, administration of the small molecule fragment induces a humoral immune response. In some embodiments, administration of the small molecule fragment induces a cell-mediated immune response. In some embodiments, administration of the small molecule fragment increases an immune response relative to a control. In some embodiments, administration of the small molecule fragment increases a humoral immune response relative to a control. In some embodiments, administration of the small molecule fragment increases a cell-mediated immune response relative to a control. In some embodiments, the control is the level of an immune response in the subject prior to administration of the small molecule fragment. In some embodiments, the control is the level of an immune response in a subject who has not been exposed to the small molecule fragment. In some embodiments, the control is the level of a humoral immune response or a cell-mediated immune response in the subject prior to administration of the small molecule fragment. In some embodiments, the control is the level of a humoral immune response or a cell-mediated immune response in a subject who has not been exposed to the small molecule fragment. In some embodiments, the cysteine-containing polypeptide is overexpressed in a disease or condition. In some embodiments, the cysteine-containing polypeptide comprises one or more mutations. In some embodiments, the cysteine-containing polypeptide comprising one or more mutations is overexpressed in a disease or condition. In some embodiments, the disease or condition is cancer. In some embodiments, the cysteine-containing polypeptide is a cancer-associated protein. In some embodiments, the cysteine-containing polypeptide is overexpressed in a cancer. In some embodiments, the cysteine-containing polypeptide comprising one or more mutations is overexpressed in a cancer. In some embodiments, the cysteine-containing polypeptide is a non-denatured form of the polypeptide. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide comprises a protein illustrated in Tables 1-5. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in FIG. 1 (e.g., FIG. 1A and/or FIG. 1B). In some embodiments, F further comprises a linker moiety that connects F to the carbonyl moiety. In some embodiments, the small molecule fragment is a small molecule fragment illustrated in FIG. 1 (e.g., FIG. 1A and/or FIG. 1B). In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the vaccine further comprises a cysteine-containing polypeptide-small molecule fragment adduct. In some embodiments, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQ ID NOs: 1-9655. In some embodiments, the vaccine further comprises an adjuvant. In some embodiments, the vaccine is formulated for parenteral, oral, or intranasal administration.

Disclosed herein, in certain embodiments, is a pharmaceutical composition comprising:
a) a cysteine-containing polypeptide covalently bond to a small molecule fragment, wherein the small molecule fragment is a small molecule fragment of Formula (I):

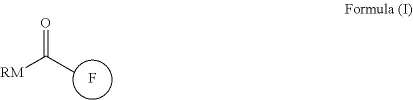

wherein:
RM is a reactive moiety selected from a Michael acceptor moiety, a leaving group moiety, or a moiety capable of forming a covalent bond with the thiol group of a cysteine residue; and
F is a small molecule fragment moiety; and wherein the small molecule fragment is covalently bond to a cysteine residue of the cysteine-containing polypeptide; and
b) an excipient.

In some embodiments, the cysteine-containing polypeptide is a non-denatured form of the polypeptide. In some embodiments, the cysteine-containing polypeptide comprises a biologically active cysteine site. In some embodiments, the biologically active cysteine site is a cysteine residue that is located about 10 Å or less to an active-site ligand or residue. In some embodiments, the cysteine residue that is located about 10 Å or less to the active-site ligand or residue is an active site cysteine. In some embodiments, the biologically active cysteine site is an active site cysteine. In some embodiments, the biologically active cysteine site is a cysteine residue that is located greater than 10 Å from an active-site ligand or residue. In some embodiments, the cysteine residue that is located greater than 10 Å from the active-site ligand or residue is a non-active site cysteine. In some embodiments, the biologically active cysteine site is a non-active site cysteine. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide is an enzyme, a transporter, a receptor, a channel protein, an adaptor protein, a chaperone, a signaling protein, transcription related protein, or translation related protein. In some embodiments, the enzyme comprises kinases, proteases, or deubiquitinating enzymes. In some embodiments, the protease is a cysteine protease. In some embodiments, the cysteine protease comprises caspases. In some embodiments, the signaling protein comprises vascular endothelial growth factor. In some embodiments, the signaling protein comprises a redox signaling protein. In some embodiments, the cysteine-containing polypeptide is about 20, 30, 35, 40, 45, 50, 55, 60, 65, 70, 75, 80, 85, 90, 95, 100 amino acid residues in length or more. In some embodiments, the cysteine-containing polypeptide comprises a protein illustrated in Tables 1-5. In some embodiments, the cysteine-containing polypeptide comprises an isolated and purified protein. In some embodiments, the isolated and purified protein is a protein illustrated in Tables 1-5. In some embodiments, the cysteine-containing polypeptide is at most 50 amino acid residues in length. In some embodiments, the cysteine-containing polypeptide comprises an isolated and purified polypeptide comprising at least 80% sequence identity to at least seven contiguous amino acids of an amino acid sequence selected from SEQ ID NOs: 1-9655. In some embodiments, the cysteine-containing polypeptide comprises an isolated and purified polypeptide comprising at least 85% sequence identity to at least seven contiguous amino acids of an amino acid sequence selected from SEQ ID NOs: 1-9655. In some embodiments, the cysteine-containing polypeptide comprises an isolated and purified polypeptide comprising at least 90% sequence identity to at least seven contiguous amino acids of an amino acid sequence selected from SEQ ID NOs: 1-9655. In some embodiments, the cysteine-containing polypeptide comprises an isolated and purified polypeptide comprising at least 95% sequence identity to at least seven contiguous amino acids of an amino acid sequence selected from SEQ ID NOs: 1-9655. In some embodiments, the cysteine-containing polypeptide comprises an isolated and purified polypeptide comprising at least 96% sequence identity to at least seven contiguous amino acids of an amino acid sequence selected from SEQ ID NOs: 1-9655. In some embodiments, the cysteine-containing polypeptide comprises an isolated and purified polypeptide comprising at least 97% sequence identity to at least seven contiguous amino acids of an amino acid sequence selected from SEQ ID NOs: 1-9655. In some embodiments, the cysteine-containing polypeptide comprises an isolated and purified polypeptide comprising at least 98% sequence identity to at least seven contiguous amino acids of an amino acid sequence selected from SEQ ID NOs: 1-9655. In some embodiments, the cysteine-containing polypeptide comprises an isolated and purified polypeptide comprising at least 99% sequence identity to at least seven contiguous amino acids of an amino acid sequence selected from SEQ ID NOs: 1-9655. In some embodiments, the cysteine-containing polypeptide comprises an isolated and purified polypeptide comprising 100% sequence identity to at least seven contiguous amino acids of an amino acid sequence selected from SEQ ID NOs: 1-9655. In some embodiments, the cysteine-containing polypeptide comprises an isolated and purified polypeptide consisting of 100% sequence identity to at least seven contiguous amino acids of an amino acid sequence selected from SEQ ID NOs: 1-9655. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in FIG. 1 (e.g., FIG. 1A and/or FIG. 1B). In some embodiments, F further comprises a linker moiety that connects F to the carbonyl moiety. In some embodiments, the small molecule fragment is a small molecule fragment illustrated in FIG. 1 (e.g., FIG. 1A and/or FIG. 1B). In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the pharmaceutical composition is formulated for parenteral, oral, or intranasal administration.

Disclosed herein, in certain embodiments, is a vaccine comprising a pharmaceutical composition disclosed above. In some embodiments, the vaccine further comprises an adjuvant. In some embodiments, the vaccine is formulated for parenteral, oral, or intranasal administration.

Disclosed herein, in certain embodiments, is an isolated and purified antibody or its binding fragment thereof comprising a heavy chain CDR1, CDR2 and CDR3 sequence and a light chain CDR1, CDR2, and CDR3 sequence, wherein the heavy chain and light chain CDRs interact with a cysteine-containing polypeptide that is covalently bond to a small molecule fragment, wherein the small molecule fragment is a small molecule fragment of Formula (I):

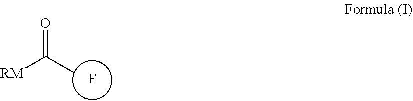

wherein:
RM is a reactive moiety selected from a Michael acceptor moiety, a leaving group moiety, or a moiety capable of forming a covalent bond with the thiol group of a cysteine residue; and
wherein the small molecule fragment is covalently bond to a cysteine residue of the cysteine-containing polypeptide.

In some embodiments, the antibody or its binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), camelid antibody, or binding fragment thereof.

Disclosed herein, in certain embodiments, is a kit comprising a pharmaceutical composition described above.

Disclosed herein, in certain embodiments, is a kit comprising an isolated and purified antibody or its binding fragment thereof disclosed above.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1B illustrate exemplary small molecule fragments described herein.

### DETAILED DESCRIPTION OF THE DISCLOSURE

Cysteine containing proteins encompass a large repertoire of proteins that participate in numerous cellular functions such as mitogenesis, proliferation, apoptosis, gene regulation, and proteolysis. These proteins include enzymes, transporters, receptors, channel proteins, adaptor proteins, chaperones, signaling proteins, plasma proteins, transcription related proteins, translation related proteins, mitochondrial proteins, or cytoskeleton related proteins. Dysregulated expression of a cysteine containing protein, in many cases, is associated with or modulates a disease, for example, such as cancer.

In some instances, small molecule compounds are capable of eliciting an immune response. In some instances, these small molecule compounds are referred to as haptens. In some cases, a hapten is a non-immunogenic compound but becomes immunogenic when it interacts with a carrier molecule such as a protein. For example, upon administration of a small molecule hapten, the hapten forms an adduct with a protein of interest in a process refers to as haptenization. In some cases, the protein-hapten adduct becomes antigenically active and enables priming of T cells and B cells, thereby directing immune response to a cell that expresses the protein of interest.

In some embodiments, disclosed herein are small molecule fragments that elicit an immune response upon interaction with cysteine-containing proteins (or cysteine-containing polypeptides). In some instances, also disclosed herein includes use of a small molecule fragment described herein to elicit or modulate an immune response in a subject. In such instances, the small molecule fragment forms an adduct with an endogenous cysteine-containing protein, and subsequently directs immune response to the cell that expresses the endogenous cysteine-containing protein. In some instances, the cell that expresses the endogenous cysteine-containing protein is a disease cell (e.g., a cancerous cell). In some instances, the endogenous cysteine-containing protein is present only in a diseased cell (e.g., a cancerous cell). In other instances, the endogenous cysteine-containing protein is overexpressed in a diseased cell (e.g., a cancerous cell) and/or comprises one or more mutations in a diseased cell (e.g., a cancerous cell).

In some embodiments, also disclosed herein are vaccines and pharmaceutical compositions that comprise one or more small molecule fragments described herein. In some instances, additionally descried herein are vaccines and pharmaceutical compositions that comprise one or more cysteine-containing polypeptide-small molecule fragment adducts or antibodies that recognize a cysteine-containing polypeptide-small molecule fragment adduct described herein.

In additional embodiments, described herein include kits for use with any of the methods, vaccines, and pharmaceutical compositions disclosed herein.

### Small Molecule Fragments

In some embodiments, described herein include pharmaceutical compositions, vaccines, and methods of use of a small molecule fragment. In some embodiments, a small molecule fragment described herein comprises a non-naturally occurring molecule. In some instances, the non-naturally occurring molecule does not include a natural and/or non-natural peptide fragment, or a small molecule that is produced naturally within the body of a mammal.

In some embodiments, a small molecule fragment described herein comprises a molecular weight of about 100 Dalton or higher. In some embodiments, a small molecule fragment comprises a molecular weight of about 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 550, 600, 650, 700, 750, 800, 850, 900, 950, 1000 Dalton, or higher. In some instances, the molecular weight of a small molecule fragment is between about 150 and about 500, about 150 and about 450, abut 150 and about 440, about 150 and about 430, about 150 and about 400, about 150 and about 350, about 150 and about 300, about 150 and about 250, about 170 and about 500, about 180 and about 450, about 190 and about 400, about 200 and about 350, about 130 and about 300, or about 120 and about 250 Dalton.

In some embodiments, the molecular weight of a small molecule fragment described herein is the molecular weight prior to enrichment with one or more elements selected from a halogen, a nonmetal, a transition metal, or a combination thereof. In some embodiments, the molecular weight of a small molecule fragment described herein is the molecular weight prior to enrichment with a halogen. In some embodiments, the molecular weight of a small molecule fragment described herein is the molecular weight prior to enrichment with a nonmetal. In some embodiments, the molecular weight of a small molecule fragment described herein is the molecular weight prior to enrichment with a transition metal.

In some embodiments, a small molecule fragment described herein comprises micromolar or millimolar binding affinity. In some instances, a small molecule fragment comprises a binding affinity of about 1 μM, 10 μM, 100 μM, 500 μM, 1 mM, 10 mM, or higher.

In some embodiments, a small molecule fragment described herein has a high ligand efficiency (LE). Ligand efficiency is the measurement of the binding energy per atom of a ligand to its binding partner. In some instances, the ligand efficiency is defined as the ratio of the Gibbs free energy (ΔG) to the number of non-hydrogen atoms of the compound (N):

LE=(ΔG)/N.

In some cases, LE is also arranged as:

LE=1.4(−log IC₅₀)/N.

In some instances, the LE score is about 0.3 kcal mol⁻¹ HA⁻¹, about 0.35 kcal mol⁻¹ HA⁻¹, about 0.4 kcal mol⁻¹ HA⁻¹, or higher.

In some embodiments, a small molecule fragment described herein is designed based on the Rule of 3. In some embodiments, the Rule of 3 comprises a non-polar solvent-polar solvent (e.g. octanol-water) partition coefficient log P of about 3 or less, a molecular mass of about 300 Daltons or less, about 3 hydrogen bond donors or less, about 3 hydrogen bond acceptors or less, and about 3 rotatable bonds or less.

In some embodiments, a small molecule fragment described herein comprises three cyclic rings or less.

In some embodiments, a small molecule fragment described herein binds to a cysteine residue of a polypeptide that is about 20 amino acid residues in length or more. In some instances, a small molecule fragment described herein binds to a cysteine residue of a polypeptide that is about 30, 35, 40, 45, 50, 55, 60, 65, 70, 75, 80, 85, 90, 95, 100 amino acid residues in length or more.

In some embodiments, a small molecule fragment described herein further comprises pharmacokinetic parameters that are unsuitable as a therapeutic agent for administration without further optimization of the small molecule fragments. In some instances, the pharmacokinetic parameters that are suitable as a therapeutic agent comprise parameters in accordance with FDA guideline, or in accordance with a guideline from an equivalent Food and Drug Administration outside of the United States. In some instances, the pharmacokinetic parameters comprise the peak plasma concentration (Cmax), the lowest concentration of a therapeutic agent (Cmin), volume of distribution, time to reach Cmax, elimination half-life, clearance, and the life. In some embodiments, the pharmacokinetic parameters of the small molecule fragments are outside of the parameters set by the FDA guideline, or by an equivalent Food and Drug Administration outside of the United States. In some instances, a skilled artisan understands, in view of the pharmacokinetic parameters of the small molecule fragments described herein, that these small molecule fragments are unsuited as therapeutic agents without further optimization.

In some embodiments, a small molecule fragment described herein comprises a reactive moiety which forms a covalent interaction with the thiol group of a cysteine residue of a cysteine-containing protein, and an affinity handle moiety.

In some instances, a small molecule fragment described herein is a small molecule fragment of Formula (I):

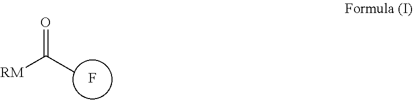

wherein:
RM is a reactive moiety selected from a Michael acceptor moiety, a leaving group moiety, or a moiety capable of forming a covalent bond with the thiol group of a cysteine residue; and F is a small molecule fragment moiety.

In some instances, the Michael acceptor moiety comprises an alkene or an alkyne moiety. In some cases, F is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some embodiments, a small molecule fragment of Formula (I) selectively interact with one or more protein variants. In some instances for example, a small molecule fragment of Formula (I) interacts or binds to the wild-type protein but does not bind to a mutant form of the protein. Conversely, in some instances, a small molecule fragment of Formula (I) interacts or binds to one specific protein mutant but does not interact with either the wild-type or the same protein comprising a different mutation. As used herein, the term “variant” comprises mutations within the protein sequence, additions or deletions of the protein sequence, and/or termini truncations. As used herein, the term “variant” comprises a protein having different conformations, for example, an active conformation or an inactive conformation. In some instances, a small molecule fragment of Formula (I) interacts with about 1, 2, 3, 4, 5, or more different variants of a protein of interest. In additional instances, a small molecule fragment of Formula (I) interacts with about 1 variant of a protein of interest. In additional instances, a small molecule fragment of Formula (I) interacts with about 2 variants of a protein of interest. In additional instances, a small molecule fragment of Formula (I) interacts with about 3 variants of a protein of interest. In additional instances, a small molecule fragment of Formula (I) interacts with about 4 variants of a protein of interest. In additional instances, a small molecule fragment of Formula (I) interacts with about 5 variants of a protein of interest.

In some embodiments, a small molecule fragment of Formula (I) does not contain a second binding site. In some instances, a small molecule fragment moiety does not bind to the protein. In some cases, a small molecule fragment moiety does not covalently bind to the protein. In some instances, a small molecule fragment moiety does not interact with a secondary binding site on the protein. In some instances, the secondary binding site is an active site such as an ATP binding site. In some cases, the active site is at least about 10, 15, 20, 25, 35, 40 Å, or more away from the biologically active cysteine residue. In some instances, the small molecule fragment moiety does not interact with an active site such as an ATP binding site.

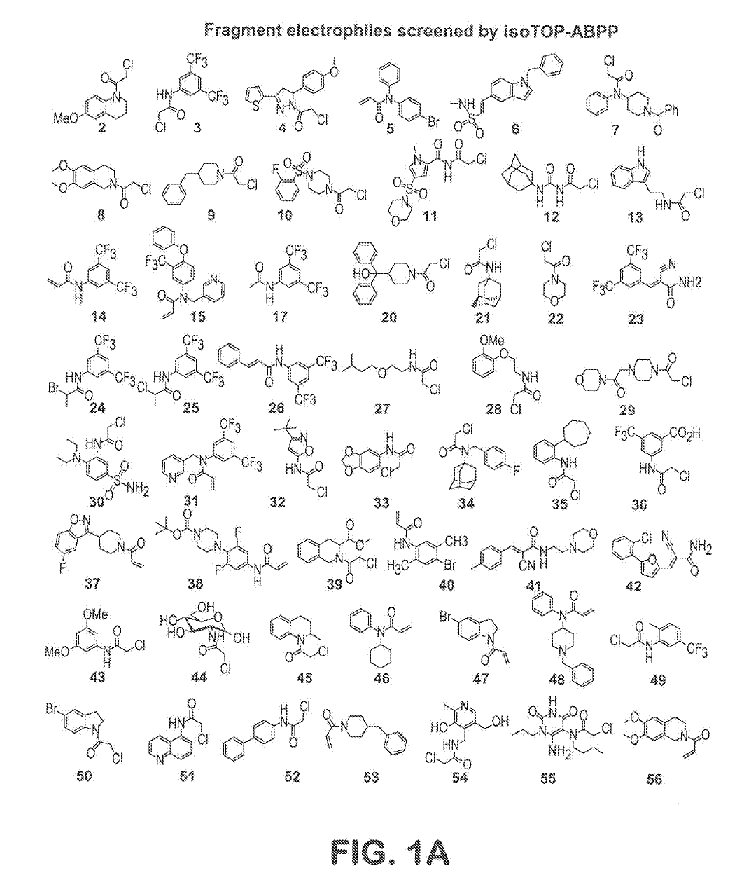

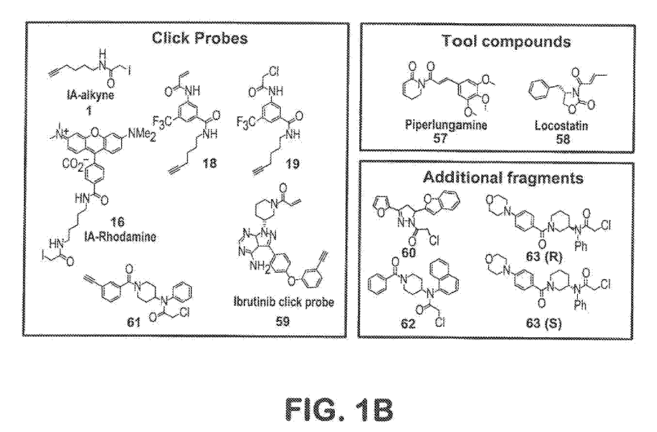

In some instances, F is a small molecule fragment moiety illustrated in FIG. 1 (e.g., FIG. 1A and/or FIG. 1B). In some instances, F is a small molecule fragment moiety illustrated in FIG. 1A. In some cases, F further comprises a linker moiety that connects F to the carbonyl moiety. In some cases, the small molecule fragment is a small molecule fragment illustrated in FIG. 1 (e.g., FIG. 1A and/or FIG. 1B).

In some instances, F is a small molecule fragment moiety selected from: N-(4-bromophenyl)-N-phenylacrylamide, N-(1-benzoylpiperidin-4-yl)-2-chloro-N-phenylacetamide, 1-(4-benzylpiperidin-1-yl)-2-chloroethan-1-one, N-(2-(1H-indol-3-yl)ethyl)-2-chloroacetamide, N-(3,5-bis(trifluoromethyl)phenyl)acrylamide, N-(4-phenoxy-3-(trifluoromethyl)phenyl)-N-(pyridin-3-ylmethyl)acrylamide, N-(3,5-bis(trifluoromethyl)phenyl)acetamide, 2-chloro-1-(4-(hydroxydiphenylmethyl)piperidin-1-yl)ethan-1-one, (E)-3-(3,5-bis(trifluoromethyl)phenyl)-2-cyanoacrylamide, N-(3,5-bis(trifluoromethyl)phenyl)-2-bromopropanamide, N-(3,5-bis(trifluoromethyl)phenyl)-2-chloropropanamide, N-(3,5-bis(trifluoromethyl)phenyl)-N-(pyridin-3-ylmethyl)acrylamide, 3-(2-chloroacetamido)-5-(trifluoromethyl)benzoic acid, I-(4-(5-fluorobenzisoxazol-3-yl)piperidin-1-yl)prop-2-en-1-one, tert-butyl 4-(4-acrylamido-2,6-difluorophenyl)piperazine-1-carboxylate, N-(4-bromo-2,5-dimethylphenyl)acrylamide, 2-chloroacetamido-2-deoxy-α/β-D-glucopyranose, 2-chloro-1-(2-methyl-3,4-dihydroquinolin-1 (2H)-yl)ethan-1-one, N-cyclohexyl-N-phenylacrylamide, 1-(5-bromoindolin-1-yl)prop-2-en-1-one, N-(1-benzylpiperidin-4-yl)-N-phenylacrylamide, 2-chloro-N-(2-methyl-5-(trifluoromethyl)phenyl)acetamide, 1-(5-bromoindolin-1-yl)-2-chloroethan-1-one, 2-chloro-N-(quinolin-5-yl)acetamide, 1-(4-benzylpiperidin-1-yl)prop-2-en-1-one, 2-chloro-N-((3-hydroxy-5-(hydroxymethyl)-2-methylpyridin-4-yl)methyl)acetamide, or 1-(6,7-dimethoxy-3,4-dihydroisoquinolin-2(1H)-yl)prop-2-en-1-one.

In some embodiments, the small molecule fragment of Formula (I) comprise a molecular weight of about 100, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 550, 600, 650, 700, 750, 800, 850, 900, 950, 1000 Dalton, or higher. In some instances, the molecular weight of the small molecule fragment of Formula (I) is between about 150 and about 500, about 150 and about 450, abut 150 and about 440, about 150 and about 430, about 150 and about 400, about 150 and about 350, about 150 and about 300, about 150 and about 250, about 170 and about 500, about 180 and about 450, about 190 and about 400, about 200 and about 350, about 130 and about 300, or about 120 and about 250 Dalton.

In some embodiments, the molecular weight of the small molecule fragment of Formula (I) is the molecular weight prior to enrichment with one or more elements selected from a halogen, a nonmetal, a transition metal, or a combination thereof. In some embodiments, the molecular weight of the small molecule fragment of Formula (I) is the molecular weight prior to enrichment with a halogen. In some embodiments, the molecular weight of the small molecule fragment of Formula (I) is the molecular weight prior to enrichment with a nonmetal. In some embodiments, the molecular weight of the small molecule fragment of Formula (I) is the molecular weight prior to enrichment with a transition metal.

In some instances, the small molecule fragment of Formula (I) comprises micromolar or millimolar binding affinity. In some instances, the small molecule fragment of Formula (I) comprises a binding affinity of about 1 μM, 10 μM, 10 μM, 500 μM, 1 mM, 10 mM, or higher.

In some cases, the small molecule fragment of Formula (I) has a LE score about 0.3 kcal mol⁻¹ HA⁻¹, about 0.35 kcal mol⁻¹ HA⁻¹, about 0.4 kcal mol⁻¹ HA⁻¹, or higher

In some embodiments, the small molecule fragment of Formula (I) follows the design parameters of Rule of 3. In some instances, the small molecule fragment of Formula (I) has a non-polar solvent-polar solvent (e.g. octanol-water) partition coefficient log P of about 3 or less, a molecular mass of about 300 Daltons or less, about 3 hydrogen bond donors or less, about 3 hydrogen bond acceptors or less, and about 3 rotatable bonds or less.

In some embodiments, the small molecule fragment of Formula (I) comprises three cyclic rings or less.

In some embodiments, the small molecule fragment of Formula (I) binds to a cysteine residue of a polypeptide (e.g., a cysteine-containing protein) that is about 20 amino acid residues in length or more. In some instances, the small molecule fragments described herein binds to a cysteine residue of a polypeptide (e.g., a cysteine-containing protein) that is about 30, 35, 40, 45, 50, 55, 60, 65, 70, 75, 80, 85, 90, 95, 100 amino acid residues in length or more.

In some instances, the small molecule fragment of Formula (I) has pharmacokinetic parameters outside of the parameters set by the FDA guideline, or by an equivalent Food and Drug Administration outside of the United States. In some instances, a skilled artisan understands in view of the pharmacokinetic parameters of the small molecule fragment of Formula (I) described herein that these small molecule fragment is unsuited as a therapeutic agent without further optimization.

### Cysteine-Containing Proteins

In some embodiments, disclosed herein include a cysteine-containing polypeptide. In some instances, the cysteine-containing polypeptide is about 20, 30, 35, 40, 45, 50, 55, 60, 65, 70, 75, 80, 85, 90, 95, 100 amino acid residues in length or more. In some instances, the cysteine-containing polypeptide is a cysteine-containing protein or its fragment thereof. In some instances, the cysteine-containing protein is a soluble protein or its fragment thereof, or a membrane protein or its fragment thereof. In some instances, the cysteine-containing protein is involved in one or more of a biological process such as protein transport, lipid metabolism, apoptosis, transcription, electron transport, mRNA processing, or host-virus interaction. In some instances, the cysteine-containing protein is associated with one or more of diseases such as cancer or one or more disorders or conditions such as immune, metabolic, developmental, reproductive, neurological, psychiatric, renal, cardiovascular, or hematological disorders or conditions.

In some embodiments, the cysteine-containing protein comprises a biologically active cysteine residue. In some embodiments, the cysteine-containing protein comprises one or more cysteines in which at least one cysteine is a biologically active cysteine residue. In some cases, the biologically active cysteine site is a cysteine residue that is located about 10 Å or less to an active-site ligand or residue. In some cases, the cysteine residue that is located about 10 Å or less to the active-site ligand or residue is an active site cysteine. In other cases, the biologically active cysteine site is a cysteine residue that is located greater than 10 Å from an active-site ligand or residue. In some instances, the cysteine residue is located greater than 12 Å, 15 Å, 20 Å, 25 Å, 30 Å, 35 Å, 40 Å, 45 Å, or greater than 50 Å from an active-site ligand or residue. In some cases, the cysteine residue that is located greater than 10 Å from the active-site ligand or residue is a non-active site cysteine. In additional cases, the cysteine-containing protein exists in an active form, or in a pro-active form.

In some embodiments, the cysteine-containing protein comprises one or more functions of an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing protein is an enzyme, a transporter, a receptor, a channel protein, an adaptor protein, a chaperone, a signaling protein, a plasma protein, transcription related protein, translation related protein, mitochondrial protein, or cytoskeleton related protein. In some instances, the cysteine-containing protein has an uncategorized function.

In some embodiments, the cysteine-containing protein is an enzyme. An enzyme is a protein molecule that accelerates or catalyzes chemical reaction. In some embodiments, non-limiting examples of enzymes include kinases, proteases, or deubiquitinating enzymes.

In some instances, exemplary kinases include tyrosine kinases such as the TEC family of kinases such as Tee, Bruton's tyrosine kinase (Btk), interleukin-2-inducible T-cell kinase (Itk) (or Emt/Tsk), Bmx, and Txk/Rlk; spleen tyrosine kinase (Syk) family such as SYK and Zeta-chain-associated protein kinase 70 (ZAP-70); Src kinases such as Src, Yes, Fyn, Fgr, Lck, Hck, Blk, Lyn, and Frk; JAK kinases such as Janus kinase 1 (JAK1), Janus kinase 2 (JAK2), Janus kinase 3 (JAK3), and Tyrosine kinase 2 (TYK2); or ErbB family of kinases such as Her1 (EGFR, ErbB1), Her2 (Neu, ErbB2), Her3 (ErbB3), and Her4 (ErbB4).

In some embodiments, the cysteine-containing protein is a protease. In some embodiments, the protease is a cysteine protease. In some cases, the cysteine protease is a caspase. In some instances, the caspase is an initiator (apical) caspase. In some instances, the caspase is an effector (executioner) caspase. Exemplary caspase includes CASP2, CASP8, CASP9, CASP10, CASP3, CASP6, CASP7, CASP4, and CASP5. In some instances, the cysteine protease is a cathepsin. Exemplary cathepsin includes Cathepsin B, Cathepsin C, CathepsinF, Cathepsin H, Cathepsin K, Cathepsin L1, Cathepsin L2, Cathepsin O, Cathepsin S, Cathepsin W, or Cathepsin Z.

In some embodiments, the cysteine-containing protein is a deubiquitinating enzyme (DUB). In some embodiments, exemplary deubiquitinating enzymes include cysteine proteases DUBs or metalloproteases. Exemplary cysteine protease DUBs include ubiquitin-specific protease (USP/UBP) such as USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46; ovarian tumor (OTU) proteases such as OTUB1 and OTUB2; Machado-Josephin domain (MJD) proteases such as ATXN3 and ATXN3L; and ubiquitin C-terminal hydrolase (UCH) proteases such as BAP1, UCHL1, UCHL3, and UCHL5. Exemplary metalloproteases include the Jab1/Mov34/Mpr1 Pad1 N-terminal+ (MPN+) (JAMM) domain proteases.

In some embodiments, exemplary cysteine-containing proteins as enzymes include, but are not limited to, Glyceraldehyde-3-phosphate dehydrogenase (GAPDH), Protein arginine N-methyltransferase 1 (PRMT1), Peptidyl-prolyl cis-trans isomerase NIMA-interaction (PIN1), Acetyl-CoA acetyltransferase (mitochondrial) (ACAT1), Glutathione S-transferase P (GSTP1), Elongation factor 2 (EEF2), Glutathione S-transferase omega-1 (GSTO1), Acetyl-CoA acetyltransferase (mitochondrial) (ACAT1), Protein disulfide-isomerase A4 (PDIA4), Prostaglandin E synthase 3 (PTGES3), Adenosine kinase (ADK), Elongation factor 2 (EEF2), Isoamyl acetate-hydrolyzing esterase 1 homolog (IAH1), Peroxiredoxin-5 (mitochondrial) (PRDX5), Inosine-5-monophosphate dehydrogenase 2 (IMPDH2), 3-hydroxyacyl-CoA dehydrogenase type-2 (HSDI7B10), Omega-amidase NIT2 (NIT2), Aldose reductase (AKR1B1), Monofunctional C1-tetrahydrofolate synthase (mitochondrial) (MTHFD1L), Protein disulfide-isomerase A6 (PDIA6), Pyruvate kinase isozymes M1/M2 (PKM), 6-phosphogluconolactonase (PGLS), Acetyl-CoA acetyltransferase (mitochondrial) (ACAT1), ERO1-like protein alpha (EROIL), Thioredoxin domain-containing protein 17 (TXNDC17), Protein disulfide-isomerase A4 (PDIA4), Protein disulfide-isomerase A3 (PDIA3), 3-ketoacyl-CoA thiolase (mitochondrial) (ACAA2), Dynamin-2 (DNM2), DNA replication licensing factor MCM3 (MCM3), Serine-tRNA ligase (cytoplasmic) (SARS), Fatty acid synthase (FASN), Acetyl-CoA acetyltransferase (mitochondrial) (ACAT1), Protein disulfide-isomerase (P4HB), Deoxycytidine kinase (DCK), Eukaryotic translation initiation factor 3 subunit (EIF3F), Protein disulfide-isomerase A6 (PDIA6), UDP-N-acetylglucosamine-peptide N-acetylglucosamine (OGT), Ketosamine-3-kinase (FN3KRP), Protein DJ-1 (PARK7), Phosphoglycolate phosphatase (PGP), DNA replication licensing factor MCM6 (MCM6), Fructose-2,6-bisphosphatase TIGAR (TIGAR), Cleavage and polyadenylation specificity factor subunit (CPSF3), Ubiquitin-conjugating enzyme E2 L3 (UBE2L3), Alanine-tRNA ligase, cytoplasmic (AARS), Mannose-1-phosphate guanyltransferase alpha (GMPPA), C-1-tetrahydrofolate synthase (cytoplasmic) (MTHFD1), Dynamin-1-like protein (DNM1L), Protein disulfide-isomerase A3 (PDIA3), Aspartyl aminopeptidase (DNPEP), Acetyl-CoA acetyltransferase (cytosolic) (ACAT2), Thioredoxin domain-containing protein 5 (TXNDC5), Thymidine kinase (cytosolic) (TK1), Inosine-5-monophosphate dehydrogenase 2 (IMPDH2), Ubiquitin carboxyl-terminal hydrolase isozyme L3 (UCHL3), Integrin-linked protein kinase (ILK), Cyclin-dependent kinase 2 (CDK2), Histone acetyltransferase type B catalytic subunit (HAT1), Enoyl-CoA delta isomerase 2 (mitochondrial) (ECI2), C-1-tetrahydrofolate synthase (cytoplasmic) (MTHFD1), Deoxycytidine kinase (DCK), Ubiquitin-like modifier-activating enzyme 6 (UBA6), Protein-L-isoaspartate (D-aspartate)O-methyltransferase (PCMT1), Monofunctional C1-tetrahydrofolate synthase (mitochondrial) (MTHFD1L), Thymidylate kinase (DTYMK), Protein ETHEl (mitochondrial) (ETHEl), Arginine-tRNA ligase (cytoplasmic) (RARS), NEDD8-activating enzyme E1 catalytic subunit (UBA3), Dual specificity mitogen-activated protein kinase (MAP2K3), Ubiquitin-conjugating enzyme E2S (UBE2S), Amidophosphoribosyltransferase (PPAT), Succinate-semialdehyde dehydrogenase (mitochondrial) (ALDH5A1), CAD, Phosphoenolpyruvate carboxykinase (PCK2), 6-phosphofructokinase type C (PFKP), Acyl-CoA synthetase family member 2 (mitochondrial) (ACSF2), Multifunctional protein ADE2 (PAICS), Desumoylating isopeptidase 1 (DESI1), 6-phosphofructokinase type C (PFKIF), V-type proton ATPase catalytic subunit A (ATP6VIA), 3-ketoacyl-CoA thiolase (peroxisomal) (ACAA1), Galactokinase (GALK1), Thymidine kinase (cytosolic) (TK1), ATPase WRNIP1 (WRNIP1), Phosphoribosylformylglycinamidine synthase (PFAS), V-type proton ATPase catalytic subunit A (ATP6V1A), Thioredoxin domain-containing protein 5 (TXNDC5), 4-trimethylaminobutyraldehyde dehydrogenase (ALDH9A1), Dual specificity mitogen-activated protein kinase (MAP2K4), Calcineurin-like phosphoesterase domain-containing (CPPED1), Dual specificity protein phosphatase 12 (DUSP12), Phosphoribosylformylglycinamidine synthase (PFAS), Diphosphomevalonate decarboxylase (MVD), D-3-phosphoglycerate dehydrogenase (PHGDH), Cell cycle checkpoint control protein RAD9A (RAD9A), Peroxiredoxin-1 (PRDX1), Sorbitol dehydrogenase (SORD), Peroxiredoxin-4 (PRDX4), AMP deaminase 2 (AMPD2), Isocitrate dehydrogenase (IDH1), Pyruvate carboxylase (mitochondrial) (PC), Integrin-linked kinase-associated serine/threonine (ILKAP), Methylmalonate-semialdehyde dehydrogenase (ALDH6A1), 26S proteasome non-ATPase regulatory subunit 14 (PSMD14), Thymidylate kinase (DTYMK), 6-phosphofructo-2-kinase/fructose-2,6-bisphosphata (PFKFB2), Peroxiredoxin-5 (mitochondrial) (PRDX5), PDP1, Cathepsin B (CTSB), Transmembrane protease serine 12 (TMPRSS12), UDP-glucose 6-dehydrogenase (UGDH), Histidine triad nucleotide-binding protein 1 (HINT1), E3 ubiquitin-protein ligase UBR5 (UBR5), SAM domain and HD domain-containing protein 1 (SAMHD1), Probable tRNA threonylcarbamoyladenosine biosynthesis (OSGEP), Methylated-DNA-protein-cysteine methyltransferase (MGMT), Fatty acid synthase (FASN), Adenosine deaminase (ADA), Cyclin-dependent kinase 19 (CDK19), Serine/threonine-protein kinase 38 (STK38), Mitogen-activated protein kinase 9 (MAPK9), tRNA (adenine(58)-N(1))-methyltransferase catalytic (TRMT61A), Glyoxylate reductase/hydroxypyruvate reductase (GRHPR), Aldehyde dehydrogenase (mitochondrial) (ALDH2), Mitochondrial-processing peptidase subunit beta (PMPCB), 3-ketoacyl-CoA thiolase, peroxisomal (ACAA1), Lysophosphatidic acid phosphatase type 6 (ACP6), Ubiquitin/ISG15-conjugating enzyme E2 L6 (UBE2L6), Caspase-8 (CASP8), 2,5-phosphodiesterase 12 (PDE12), Thioredoxin domain-containing protein 12 (TXNDC12), Nitrilase homolog 1 (NIT1), ERO1-like protein alpha (ERO1L), SUMO-activating enzyme subunit 1 (SAE1), Leucine-tRNA ligase (cytoplasmic) (LARS), Protein-glutamine gamma-glutamyltransferase 2 (TGM2), Probable DNA dC-dU-editing enzyme APOBEC-3C (APOBEC3C), Double-stranded RNA-specific adenosine deaminase (ADAR), Isocitrate dehydrogenase (IDH2), Methylcrotonoyl-CoA carboxylase beta chain (mitochondrial) (MCCC2), Uridine phosphorylase 1 (UPP1), Glycogen phosphorylase (brain form) (PYGB), E3 ubiquitin-protein ligase UBR5 (UBR5), Procollagen-lysine,2-oxoglutarate 5-dioxygenase 1 (PLOD1), Ubiquitin carboxyl-terminal hydrolase 48 (USP48), Aconitate hydratase (mitochondrial) (ACO2), GMP reductase 2 (GMPR2), Pyrroline-5-carboxylate reductase 1 (mitochondrial) (PYCR1), Cathepsin Z (CTSZ), E3 ubiquitin-protein ligase UBR2 (UBR2), Cysteine protease ATG4B (ATG4B), Serine/threonine-protein kinase Nek9 (NEK9), Lysine-specific demethylase 4B (KDM4B), Insulin-degrading enzyme (IDE), Dipeptidyl peptidase 9 (DPP9), Decaprenyl-diphosphate synthase subunit 2 (PDSS2), TFIIH basal transcription factor complex helicase (ERCC3), Methionine-R-sulfoxide reductase B2 (mitochondrial) (MSRB2), E3 ubiquitin-protein ligase BRE1B (RNF40), Thymidylate synthase (TYMS), Cyclin-dependent kinase 5 (CDK5), Bifunctional 3-phosphoadenosine 5-phosphosulfate (PAPSS2), Short/branched chain specific acyl-CoA dehydrogenase (ACADSB), Cathepsin D (CTSD), E3 ubiquitin-protein ligase HUWE1 (HUWE1), Calpain-2 catalytic subunit (CAPN2), Dual specificity mitogen-activated protein kinase (MAP2K7), Mitogen-activated protein kinase kinase kinase MLT (MLTK), Bleomycin hydrolase (BLMH), Probable ATP-dependent RNA helicase DDX59 (DDX59), Cystathionine gamma-lyase (CTH), S-adenosylmethionine synthase isoform type-2 (MAT2A), 6-phosphofructokinase type C (PFKP), Cytidine deaminase (CDA), DNA-directed RNA polymerase II subunit RPB2 (POLR2B), Protein disulfide-isomerase (P4HB), Procollagen-lysine,2-oxoglutarate 5-dioxygenase 3 (PLOD3), Nucleoside diphosphate-linked moiety X motif 8 (mitochondrial) (NUDT8), E3 ubiquitin-protein ligase HUWE1 (HUWE1), Methylated-DNA-protein-cysteine methyltransferase (MGMT), Nitrilase homolog 1 (NIT1), Interferon regulatory factor 2-binding protein 1 (IRF2BP1), Ubiquitin carboxyl-terminal hydrolase 16 (USP16), Glycylpeptide N-tetradecanoyltransferase 2 (NMT2), Cyclin-dependent kinase inhibitor 3 (CDKN3), Hydroxysteroid dehydrogenase-like protein 2 (HSDL2), Serine/threonine-protein kinase VRK1 (VRK1), Serine/threonine-protein kinase A-Raf (ARAF), ATP-citrate synthase (ACLY), Probable ribonuclease ZC3H12D (ZC3HI2D), Peripheral plasma membrane protein CASK (CASK), DNA polymerase epsilon subunit 3 (POLE3), Aldehyde dehydrogenase X (mitochondrial) (ALDHIB1), UDP-N-acetylglucosamine transferase subunit ALG3 (ALG3), Protein disulfide-isomerase A4 (PDIA4), DNA polymerase alpha catalytic subunit (POLA1), Ethylmalonyl-CoA decarboxylase (ECHDC 1), Protein-tyrosine kinase 2-beta (PTK2B), E3 SUMO-protein ligase RanBP2 (RANBP2), Legumain (LGMN), Non-specific lipid-transfer protein (SCP2), Long-chain-fatty-acid-CoA ligase 4 (ACSL4), Dual specificity protein phosphatase 12 (DUSP12), Oxidoreductase HTATIP2 (HTATIP2), Serine/threonine-protein kinase MRCK beta (CDC42BPB), Histone-lysine N-methyltransferase EZH2 (EZH2), Non-specific lipid-transfer protein (SCP2), Dual specificity mitogen-activated protein kinase (MAP2K7), Ubiquitin carboxyl-terminal hydrolase 28 (USP28), 6-phosphofructokinase (liver type) (PFKL), SWI/SNF-related matrix-associated actin-dependent (SMARCAD1), Protein phosphatase methylesterase 1 (PPME1), DNA replication licensing factor MCM5 (MCM5), 6-phosphofructo-2-kinase/fructose-2,6-bisphosphata (PFKFB4), Dehydrogenase/reductase SDR family member 11 (DHRS11), Pyroglutamyl-peptidase 1 (PGPEP1), Probable E3 ubiquitin-protein ligase (MYCBP2), DNA fragmentation factor subunit beta (DFFB), Deubiquitinating protein VCIP135 (VCPIP1), Putative transferase CAF17 (mitochondrial) (IBA57), Calpain-7 (CAPN7), GDP-L-fucose synthase (TSTA3), Protein disulfide-isomerase A4 (PDIA4, Probable ATP-dependent RNA helicase (DDX59), RNA exonuclease 4 (REXO4), PDK1, E3 SUMO-protein ligase (PIAS4), DNA (cytosine-5)-methyltransferase 1 (DNMT1), Alpha-aminoadipic semialdehyde dehydrogenase (ALDH7A1), Hydroxymethylglutaryl-CoA synthase (cytoplasmic) (HMGCS1), E3 ubiquitin-protein ligase (SMURF2), Aldehyde dehydrogenase X (mitochondrial) (ALDHIB1), Tyrosine-protein kinase (BTK), DNA repair protein RAD50 (RAD50), ATP-binding domain-containing protein 4 (ATPBD4), Nucleoside diphosphate kinase 3 (NME3), Interleukin-1 receptor-associated kinase 1 (IRAK1), Ribonuclease P/MRP protein subunit POP5 (POP5), Peptide-N(4)-(N-acetyl-beta-glucosaminyl)asparagin (NGLY1), Caspase-2 (CASP2), Ribosomal protein S6 kinase alpha-3 (RPS6KA3), E3 ubiquitin-protein ligase UBR1 (UBR1), Serine/threonine-protein kinase Chk2 (CHEK2), Phosphatidylinositol 3,4,5-trisphosphate 5-phospha (INPPL1), Histone acetyltransferase p300 (EP300), Creatine kinase U-type (mitochondrial) (CKMT1B), E3 ubiquitin-protein ligase TRIM33 (TRIM33), Cancer-related nucleoside-triphosphatase (NTPCR), Aconitate hydratase (mitochondrial) (ACO2), Ubiquitin carboxyl-terminal hydrolase 34 (USP34), Probable E3 ubiquitin-protein ligase HERC4 (HERC4), E3 ubiquitin-protein ligase HECTD1 (HECTD1), Peroxisomal 2,4-dienoyl-CoA reductase (DECR2), Helicase ARIP4 (RAD54L2), Ubiquitin-like modifier-activating enzyme 7 (UBA7), ER degradation-enhancing alpha-mannosidase-like 3 (EDEM3), Ubiquitin-conjugating enzyme E20 (UBE20), Dual specificity mitogen-activated protein kinase (MAP2K7), Myotubularin-related protein 1 (MTMR1), Calcium-dependent phospholipase A2 (PLA2G5), Mitotic checkpoint serine/threonine-protein kinase (BUB1B), Putative transferase CAF17 (mitochondrial) (IBA57), Tyrosine-protein kinase ZAP-70 (ZAP70), E3 ubiquitin-protein ligase pellino homolog 1 (PELI1), Neuropathy target esterase (PNPLA6), Ribosomal protein S6 kinase alpha-3 (RPS6KA3), N6-adenosine-methyltransferase 70 kDa subunit (METTL3), Fructosamine-3-kinase (FN3K), Ubiquitin carboxyl-terminal hydrolase 22 (USP22), Rab3 GTPase-activating protein catalytic subunit (RAB3GAP1), Caspase-5 (CASP5), L-2-hydroxyglutarate dehydrogenase (mitochondrial) (L2HGDH), Saccharopine dehydrogenase-like oxidoreductase (SCCPDH), FLAD1 FAD synthase, Lysine-specific demethylase 3A (KDM3A), or Ubiquitin carboxyl-terminal hydrolase 34 (USP34).

In some embodiments, the cysteine-containing protein is a signaling protein. In some instances, exemplary signaling protein includes vascular endothelial growth factor (VEGF) proteins or proteins involved in redox signaling. Exemplary VEGF proteins include VEGF-A, VEGF-B, VEGF-C, VEGF-D, and PGF. Exemplary proteins involved in redox signaling include redox-regulatory protein FAM213A.

In some embodiments, the cysteine-containing protein is a transcription factor or regulator. Exemplary cysteine-containing proteins as transcription factors and regulators include, but are not limited to, 40S ribosomal protein S3 (RPS3), Basic leucine zipper and W2 domain-containing protein (BZW1), Poly(rC)-binding protein 1 (PCBP1), 40S ribosomal protein S11 (RPS11), 40S ribosomal protein S4, X isoform (RPS4X), Signal recognition particle 9 kDa protein (SRP9), Non-POU domain-containing octamer-binding protein (NONO), N-alpha-acetyltransferase 15, NatA auxiliary subunit (NAA15), Cleavage stimulation factor subunit 2 (CSTF2), Lamina-associated polypeptide 2, isoform alpha (TMPO), Heterogeneous nuclear ribonucleoprotein R (HNRNPR), MMS19 nucleotide excision repair protein homolog (MMS19), SWI/SNF complex subunit SMARCC2 (SMARCC2), Enhancer of mRNA-decapping protein 3 (EDC3), H/ACA ribonucleoprotein complex subunit 2 (NHP2), WW domain-containing adapter protein with coiled-c (WAC), N-alpha-acetyltransferase 15 NatA auxiliary subunit (NAA15), 40S ribosomal protein S11 (RPS11), Signal transducer and activator of transcription 1 (STAT1), Mediator of RNA polymerase II transcription subunit (MED15), Lamina-associated polypeptide 2 (isoform alpha) (TMPO), MMS19 nucleotide excision repair protein homolog (MMS19), DNA mismatch repair protein Msh2 (MSH2), Recombining binding protein suppressor of hairless (RBPJ), Mediator of RNA polymerase II transcription subunit (MED17), Heterogeneous nuclear ribonucleoprotein U (HNRNPU), Transcription initiation factor IIA subunit 2 (GTF2A2), Chromatin accessibility complex protein 1 (CHRAC1), CDKN2A-interacting protein (CDKN2AIP), Zinc finger protein 217 (ZNF217), Signal transducer and activator of transcription 3 (STAT3), WD repeat and HMG-box DNA-binding protein 1 (WDHD1), Lamina-associated polypeptide 2 (isoform alpha) (TMPO), Lamina-associated polypeptide 2 (isoforms beta/gam) (TMPO), Interferon regulatory factor 4 (IRF4), Protein flightless-1 homolog (FLI1), Heterogeneous nuclear ribonucleoprotein F (HNRNPF), Nucleus accumbens-associated protein 1 (NACC1), Transcription elongation regulator 1 (TCERG1), Protein HEXIM1 (HEXIM1), Enhancer of mRNA-decapping protein (EDC3), Zinc finger protein Aiolos (IKZF3), Transcription elongation factor SPT5 (SUPT5H), Forkhead box protein K1 (FOXK1), LIM domain-containing protein 1 (LIMD1), MMS19 nucleotide excision repair protein homolog (MMS19), Elongator complex protein 4 (ELP4), Ankyrin repeat and KH domain-containing protein 1 (ANKHD1), PML, Nuclear factor NF-kappa-B p100 subunit (NFKB2), Heterogeneous nuclear ribonucleoprotein L-like (HNRPLL), CCR4-NOT transcription complex subunit 3 (CNOT3), Constitutive coactivator of PPAR-gamma-like protein (FAMI20A), Mediator of RNA polymerase II transcription subunit (MED15), 60S ribosomal protein L7 (RPL7), Interferon regulatory factor 8 (IRF8), COUP transcription factor 2 (NR2F2), Mediator of RNA polymerase II transcription subunit (MED1), tRNA (uracil-5-)-methyltransferase homolog A (TRMT2A), Transcription factor p65 (RELA), Exosome complex component RRP42 (EXOSC7), General transcription factor 3C polypeptide 1 (GTF3C1), Mothers against decapentaplegic homolog 2 (SMAD2), Ankyrin repeat domain-containing protein 17 (ANKRD17), MMS19 nucleotide excision repair protein homolog (MMS19), Death domain-associated protein 6 (DAXX), Zinc finger protein 318 (ZNF318), Thioredoxin-interacting protein (TXNIP), Glucocorticoid receptor (NR3C1), Iron-responsive element-binding protein 2 (IREB2), Zinc finger protein 295 (ZNF295), Polycomb protein SUZ12 (SUZ12), Cleavage stimulation factor subunit 2 tau variant (CSTF2T), C-myc promoter-binding protein (DENND4A), Pinin (PNN), Mediator of RNA polymerase II transcription subunit (MED9), POU domain, class 2, transcription factor 2 (POU2F2), Enhancer of mRNA-decapping protein 3 (EDC3), A-kinase anchor protein 1 (mitochondrial) (AKAP1), Transcription factor RelB (RELB), RNA polymerase II-associated protein 1 (RPAP1), Zinc finger protein 346 (ZNF346), Chromosome-associated kinesin KIF4A (KIF4A), Mediator of RNA polymerase II transcription subunit (MED12), Protein NPAT (NPAT), Leucine-rich PPR motif-containing protein (mitochondrial) (LRPPRC), AT-hook DNA-binding motif-containing protein 1 (AHDC1), Mediator of RNA polymerase II transcription subunit (MED12), Bromodomain-containing protein 8 (BRD8), Trinucleotide repeat-containing gene 6B protein (TNRC6B), Aryl hydrocarbon receptor nuclear translocator (ARNT), Activating transcription factor 7-interacting protein (ATF7IP), Glucocorticoid receptor (NR3C1), Chromosome transmission fidelity protein 18 homolog (CHTF18), or C-myc promoter-binding protein (DENND4A).

In some embodiments, the cysteine-containing protein is a channel, transporter, or receptor. Exemplary cysteine-containing proteins as channels, transporters, or receptors include, but are not limited to, Chloride intracellular channel protein 4 (CLIC4), Exportin-1 (XPO1), Thioredoxin (TXN), Protein SEC13 homolog (SEC13), Chloride intracellular channel protein 1 (CLIC1), Guanine nucleotide-binding protein subunit beta-2 (GNB2L1), Sorting nexin-6 (SNX6), Conserved oligomeric Golgi complex subunit 3 (COG3), Nuclear cap-binding protein subunit 1 (NCBP1), Cytoplasmic dynein 1 light intermediate chain 1 (DYNC1LI1), MOB-like protein phocein (MOB4), Programmed cell death 6-interacting protein (PDCD6IP), Glutaredoxin-1 (GLRX), ATP synthase subunit alpha (mitochondrial) (ATP5A1), Treacle protein (TCOF1), Dynactin subunit 1 (DCTN1), Importin-7 (IPO7), Exportin-2 (CSE1L), ATP synthase subunit gamma (mitochondrial) (ATP5C1), Trafficking protein particle complex subunit 5 (TRAPPC5), Thioredoxin mitochondrial (TXN2), THO complex subunit 6 homolog (THOC6), Exportin-1 (XPO1), Nuclear pore complex protein Nup50 (NUP50), Treacle protein (TCOF1), Nuclear pore complex protein Nup93 (NUP93), Nuclear pore glycoprotein p62 (NUP62), Cytoplasmic dynein 1 heavy chain 1 (DYNC1H1), Thioredoxin-like protein 1 (TXNL1), Nuclear pore complex protein Nup214 (NUP214), Protein lin-7 homolog C (LIN7C), ADP-ribosylation factor-binding protein GGA2 (GGA2), Trafficking protein particle complex subunit 4 (TRAPPC4), Protein quaking (QK1), Perilipin-3 (PLIN3), Copper transport protein ATOX1 (ATOX1), Unconventional myosin-Ic (MYO1C), Nucleoporin NUP53 (NUP35), Vacuolar protein sorting-associated protein 18 homolog (VPS18), Dedicator of cytokinesis protein 7 (DOCK7), Nucleoporin p54 (NUP54), Ras-related GTP-binding protein C (RRAGC), Arf-GAP with Rho-GAP domain (ANK repeat and PH domain) (ARAP1), Exportin-5 (XPO5), Kinectin (KTN1), Chloride intracellular channel protein 6 (CLIC6), Voltage-gated potassium channel subunit beta-2 (KCNAB2), Exportin-5 (XPO5), Ras-related GTP-binding protein C (RRAGC), Ribosome-binding protein 1 (RRBP1), Acyl-CoA-binding domain-containing protein 6 (ACBD6), Chloride intracellular channel protein 5 (CLIC5), Pleckstrin homology domain-containing family A member (PLEKHA2), ADP-ribosylation factor-like protein 3 (ARL3), Protein transport protein Sec24C (SEC24C), Voltage-dependent anion-selective channel protein (VDAC3), Programmed cell death 6-interacting protein (PDCD6IP), Chloride intracellular channel protein 3 (CLIC3), Multivesicular body subunit 12A (FAM125A), Eukaryotic translation initiation factor 4E transporter (EIF4ENIF1), NmrA-like family domain-containing protein 1 (NMRAL1), Nuclear pore complex protein Nup98-Nup96 (NUP98), Conserved oligomeric Golgi complex subunit 1 (COG1), Importin-4 (IPO4), Pleckstrin homology domain-containing family A member (PLEKHA2), Cytoplasmic dynein 1 heavy chain 1 (DYNC1H1), DENN domain-containing protein 1C (DENND1C), Cytoplasmic dynein 1 heavy chain 1 (DYNC1H1), Protein ELYS (AHCTF1), Trafficking protein particle complex subunit 1 (TRAPPC1), Guanine nucleotide-binding protein-like 3 (GNL3), or Importin-13 (IPO13).

In some embodiments, the cysteine-containing protein is a chaperone. Exemplary cysteine-containing proteins as chaperones include, but are not limited to, 60 kDa heat shock protein (mitochondrial) (HSPD1), T-complex protein 1 subunit eta (CCT7), T-complex protein 1 subunit epsilon (CCT5), Heat shock 70 kDa protein 4 (HSPA4), GrpE protein homolog 1 (mitochondrial) (GRPEL1), Tubulin-specific chaperone E (TBCE), Protein unc-45 homolog A (UNC45A), Serpin H1 (SERPINH1), Tubulin-specific chaperone D (TBCD), Peroxisomal biogenesis factor 19 (PEXI9), BAG family molecular chaperone regulator 5 (BAGS), T-complex protein 1 subunit theta (CCT8), Protein canopy homolog 3 (CNPY3), DnaJ homolog subfamily C member 10 (DNAJC10), ATP-dependent Clp protease ATP-binding subunit clp (CLPX), or Midasin (MDN1).

In some embodiments, the cysteine-containing protein is an adapter, scaffolding, or modulator protein. Exemplary cysteine-containing proteins as adapter, scaffolding, or modulator proteins include, but are not limited to, Proteasome activator complex subunit 1 (PSME1), TIP41-like protein (TIPRL), Crk-like protein (CRKL), Cofilin-1 (CFL1), Condensin complex subunit 1 (NCAPD2), Translational activator GCN1 (GCNIL1), Serine/threonine-protein phosphatase 2A 56 kDa regulatory (PPP2R5D), UPF0539 protein C7orf59 (C7orf59), Protein diaphanous homolog 1 (DIAPH1), Protein asunder homolog (Asun), Ras GTPase-activating-like protein IQGAP1 (IQGAP1), Sister chromatid cohesion protein PDS5 homolog A (PDS5A), Reticulon-4 (RTN4), Proteasome activator complex subunit 4 (PSME4), Condensin complex subunit 2 (NCAPH), Sister chromatid cohesion protein PDS5 homolog A (PDS5A), cAMP-dependent protein kinase type I-alpha regulatory (PRKAR1A), Host cell factor 1 (HCFC1), Serine/threonine-protein phosphatase 4 regulatory (PPP4R2), Apoptotic chromatin condensation inducer in the nucleus (ACIN1), BRISC and BRCA1-A complex member 1 (BABAM1), Interferon-induced protein with tetratricopeptide (IFIT3), Ras association domain-containing protein 2 (RASSF2), Hsp70-binding protein 1 (HSPBP1), TBC1 domain family member 15 (TBC1D15), Dynamin-binding protein (DNMBP), Condensin complex subunit 1 (NCAPD2), Beta-2-syntrophin (SNTB2), Disks large homolog 1 (DLG1), TBC1 domain family member 13 (TBC1D13), Formin-binding protein 1-like (FNBP1L), Translational activator GCN1 (GCN1L1), GRB2-related adapter protein (GRAP), G2/mitotic-specific cyclin-B1 (CCNB 1), Myotubularin-related protein 12 (MTMR12), Protein FADD (FADD), Translational activator GCN1 (GCN1L1), Wings apart-like protein homolog (WAPAL), cAMP-dependent protein kinase type U-beta regulatory (PRKAR2B), Malcavernin (CCM2), MPP1 55 kDa erythrocyte membrane protein, Actin filament-associated protein 1 (AFAP1), Tensin-3 (TNS3), tRNA methyltransferase 112 homolog (TRMT112), Symplekin (SYMPK), TBC1 domain family member 2A (TBC1D2), ATR-interacting protein (ATRIP), Ataxin-10 (ATXN10), Succinate dehydrogenase assembly factor 2 (mitochondrial) (SDHAF2), Formin-binding protein 1 (FNBP1), Myotubularin-related protein 12 (MTMR12), Interferon-induced protein with tetratricopeptide (IFIT3), Protein CBFA2T2 (CBFA2T2), Neutrophil cytosol factor 1 (NCF1), or Protein syndesmos (NUDT16L1).

In some embodiments, a cysteine-containing polypeptide comprises a protein illustrated in Tables 1-5. In some instances, a cysteine-containing polypeptide comprises a protein illustrated in Table 1. In some instances, a cysteine-containing polypeptide comprises a protein illustrated in Table 2. In some instances, a cysteine-containing polypeptide comprises a protein illustrated in Table 3. In some instances, a cysteine-containing polypeptide comprises a protein illustrated in Table 4. In some instances, a cysteine-containing polypeptide comprises a protein illustrated in Table 5.

In some embodiments, a cysteine-containing polypeptide comprises a protein illustrated in Tables 6A-6E. In some instances, a cysteine-containing polypeptide comprises a protein illustrated in Table 6A. In some instances, a cysteine-containing polypeptide comprises a protein illustrated in Table 6B. In some instances, a cysteine-containing polypeptide comprises a protein illustrated in Table 6C. In some instances, a cysteine-containing polypeptide comprises a protein illustrated in Table 6D. In some instances, a cysteine-containing polypeptide comprises a protein illustrated in Table 6E.

In some embodiments, a cysteine-containing polypeptide comprises cereblon. Cereblon is a substrate receptor that interacts with the protein adaptor damaged DNA binding protein 1 (DDB1), scaffold Cullin-4A (CUL4A), and regulator of cullins 1 (ROC1) to form an E3 ubiquitin ligase complex. The cereblon-E3 ligase complex is involved in targeting a plurality of substrates for ubiquitination, which are then subsequently degraded by proteasomes. In some instances, thalidomide and related immunomodulatory (IMiD) compounds such as lenalidomide and pomalidomide promote and modulate cereblon recruitment of neosubstrates. For example, a cereblon modulator CC-220 has been shown to improve degradation of Ikaros and Aiolos, two zinc finger transcription factors that have been implicated in lymphoid development and differentiation (Matyskiela, et al., “A cereblon modulator (CC-220) with improved degradation of Ikaros and Aiolos,” J Med Chem. Apr. 20, 2017). Further, dBET1, a bifunctional phthalimide-conjugated ligand which is a substrate for cereblon, also selectively targets BRD4, a transcriptional coactivator, for degradation.

In some instances, cereblon is a eukaryotic protein ranging from 400-600 residues in length. The human cereblon (SEQ ID NO: 9665), which is about 442 residues in length, is encoded by the CRBN gene. The cereblon protein comprises a central LON domain (residues 80-317) followed by a C-terminal CULT domain. The LON domain is further subdivided into an N-terminal LON-N subdomain, a four helix bundle, and a C-terminal LON-C subdomain.

In some embodiments, a small molecule fragment described herein binds to a cysteine residue within cereblon. In some cases, a small molecule fragment described herein binds to a cysteine residue in the LON domain of cereblon. In some cases, a small molecule fragment described herein binds to a cysteine residue in the LON-N domain of cereblon. In some cases, a small molecule fragment described herein binds to a cysteine residue in the LON-C domain of cereblon. In other cases, a small molecule fragment described herein binds to a cysteine residue in the CULT domain of cereblon. In some instances, a small molecule fragment described herein binds to cysteine residue 188 of cereblon, wherein residue position 188 corresponds to position 188 of SEQ ID NO: 9665. In some instances, a small molecule fragment described herein binds to cysteine residue 287 of cereblon, wherein residue position 287 corresponds to position 287 of SEQ ID NO: 9665.

In some embodiments, described herein is a method of modulating cereblon activity, which comprises contacting a cell expressing cereblon with a small molecule fragment of Formula (I):

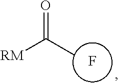

wherein RM is a reactive moiety selected from a Michael acceptor moiety, a leaving group moiety, or a moiety capable of forming a covalent bond with the thiol group of cysteine residue; and F is a small molecule fragment moiety; wherein the small molecule fragment of Formula (I) covalently binds to residue 187 or residue 288 of cereblon; and wherein residue positions 187 and 288 correspond to positions 187 and 288 of SEQ ID NO: 9665. In some instances, F is a small molecule fragment moiety illustrated in FIG. 1 (e.g., FIG. 1A and/or FIG. 1B). In some instances, F optionally comprises a second reactive moiety. In some cases, the cell is a mammalian cell. In some cases, the method is an in vivo method.

### Polypeptides Comprising a Cysteine Interacting Site

In some embodiments, a cysteine-containing polypeptide comprises a polypeptide that is at most 50 amino acid residues in length. In some embodiments, a cysteine-containing polypeptide comprises an isolated and purified polypeptide comprising at least 70% sequence identity to at least seven contiguous amino acids of an amino acid sequence selected from SEQ ID NOs: 1-9655. In some embodiments, a cysteine-containing polypeptide comprises an isolated and purified polypeptide comprising at least 75% sequence identity to at least seven contiguous amino acids of an amino acid sequence selected from SEQ ID NOs: 1-9655. In some embodiments, a cysteine-containing polypeptide comprises an isolated and purified polypeptide comprising at least 80% sequence identity to at least seven contiguous amino acids of an amino acid sequence selected from SEQ ID NOs: 1-9655. In some embodiments, a cysteine-containing polypeptide comprises an isolated and purified polypeptide comprising at least 85% sequence identity to at least seven contiguous amino acids of an amino acid sequence selected from SEQ ID NOs: 1-9655. In some embodiments, a cysteine-containing polypeptide comprises an isolated and purified polypeptide comprising at least 90% sequence identity to at least seven contiguous amino acids of an amino acid sequence selected from SEQ ID NOs: 1-9655. In some embodiments, a cysteine-containing polypeptide comprises an isolated and purified polypeptide comprising at least 91% sequence identity to at least seven contiguous amino acids of an amino acid sequence selected from SEQ ID NOs: 1-9655. In some embodiments, a cysteine-containing polypeptide comprises an isolated and purified polypeptide comprising at least 92% sequence identity to at least seven contiguous amino acids of an amino acid sequence selected from SEQ ID NOs: 1-9655. In some embodiments, a cysteine-containing polypeptide comprises an isolated and purified polypeptide comprising at least 93% sequence identity to at least seven contiguous amino acids of an amino acid sequence selected from SEQ ID NOs: 1-9655. In some embodiments, a cysteine-containing polypeptide comprises an isolated and purified polypeptide comprising at least 94% sequence identity to at least seven contiguous amino acids of an amino acid sequence selected from SEQ ID NOs: 1-9655. In some embodiments, a cysteine-containing polypeptide comprises an isolated and purified polypeptide comprising at least 95% sequence identity to at least seven contiguous amino acids of an amino acid sequence selected from SEQ ID NOs: 1-9655. In some embodiments, a cysteine-containing polypeptide comprises an isolated and purified polypeptide comprising at least 96% sequence identity to at least seven contiguous amino acids of an amino acid sequence selected from SEQ ID NOs: 1-9655. In some embodiments, a cysteine-containing polypeptide comprises an isolated and purified polypeptide comprising at least 97% sequence identity to at least seven contiguous amino acids of an amino acid sequence selected from SEQ ID NOs: 1-9655. In some embodiments, a cysteine-containing polypeptide comprises an isolated and purified polypeptide comprising at least 98% sequence identity to at least seven contiguous amino acids of an amino acid sequence selected from SEQ ID NOs: 1-9655. In some embodiments, a cysteine-containing polypeptide comprises an isolated and purified polypeptide comprising at least 99% sequence identity to at least seven contiguous amino acids of an amino acid sequence selected from SEQ ID NOs: 1-9655. In some embodiments, a cysteine-containing polypeptide comprises an isolated and purified polypeptide comprising 100% sequence identity to at least seven contiguous amino acids of an amino acid sequence selected from SEQ ID NOs: 1-9655. In some embodiments, a cysteine-containing polypeptide comprises an isolated and purified polypeptide consisting of 100% sequence identity to at least seven contiguous amino acids of an amino acid sequence selected from SEQ ID NOs: 1-9655.

In some embodiments, a cysteine-containing polypeptide comprises an isolated and purified polypeptide comprising at least 70%, 75%, 80%, 85%, 90%, 91%, 92%, 93%, 94%, 95%, 96%, 97%, 98%, or 99% sequence identity to at least seven contiguous amino acids of SEQ ID NO: 1607. In some embodiments, a cysteine-containing polypeptide comprises an isolated and purified polypeptide comprising at least 70% sequence identity to at least seven contiguous amino acids of SEQ ID NO: 1607. In some embodiments, a cysteine-containing polypeptide comprises an isolated and purified polypeptide comprising at least 75% sequence identity to at least seven contiguous amino acids of SEQ ID NO: 1607. In some embodiments, a cysteine-containing polypeptide comprises an isolated and purified polypeptide comprising at least 80% sequence identity to at least seven contiguous amino acids of SEQ ID NO: 1607. In some embodiments, a cysteine-containing polypeptide comprises an isolated and purified polypeptide comprising at least 85% sequence identity to at least seven contiguous amino acids of SEQ ID NO: 1607. In some embodiments, a cysteine-containing polypeptide comprises an isolated and purified polypeptide comprising at least 90% sequence identity to at least seven contiguous amino acids of SEQ ID NO: 1607. In some embodiments, a cysteine-containing polypeptide comprises an isolated and purified polypeptide comprising at least 91% sequence identity to at least seven contiguous amino acids of SEQ ID NO: 1607. In some embodiments, a cysteine-containing polypeptide comprises an isolated and purified polypeptide comprising at least 92% sequence identity to at least seven contiguous amino acids of SEQ ID NO: 1607. In some embodiments, a cysteine-containing polypeptide comprises an isolated and purified polypeptide comprising at least 93% sequence identity to at least seven contiguous amino acids of SEQ ID NO: 1607. In some embodiments, a cysteine-containing polypeptide comprises an isolated and purified polypeptide comprising at least 94% sequence identity to at least seven contiguous amino acids of SEQ ID NO: 1607. In some embodiments, a cysteine-containing polypeptide comprises an isolated and purified polypeptide comprising at least 95% sequence identity to at least seven contiguous amino acids of SEQ ID NO: 1607. In some embodiments, a cysteine-containing polypeptide comprises an isolated and purified polypeptide comprising at least 96% sequence identity to at least seven contiguous amino acids of SEQ ID NO: 1607. In some embodiments, a cysteine-containing polypeptide comprises an isolated and purified polypeptide comprising at least 97% sequence identity to at least seven contiguous amino acids of SEQ ID NO: 1607. In some embodiments, a cysteine-containing polypeptide comprises an isolated and purified polypeptide comprising at least 98% sequence identity to at least seven contiguous amino acids of SEQ ID NO: 1607. In some embodiments, a cysteine-containing polypeptide comprises an isolated and purified polypeptide comprising at least 99% sequence identity to at least seven contiguous amino acids of SEQ ID NO: 1607. In some embodiments, a cysteine-containing polypeptide comprises an isolated and purified polypeptide comprising 100% sequence identity to at least seven contiguous amino acids of SEQ ID NO: 1607.

In some embodiments, a cysteine-containing polypeptide comprises an isolated and purified polypeptide comprising at least 70%, 75%, 80%, 85%, 90%, 91%, 92%, 93%, 94%, 95%, 96%, 97%, 98%, or 99% sequence identity to SEQ ID NO: 1607. In some embodiments, a cysteine-containing polypeptide comprises an isolated and purified polypeptide comprising 100% sequence identity to SEQ ID NO: 1607. In some embodiments, a cysteine-containing polypeptide comprises an isolated and purified polypeptide consisting of 100% sequence identity to SEQ ID NO: 1607.

In some embodiments, a cysteine-containing polypeptide comprises an isolated and purified polypeptide comprising at least 70%, 75%, 80%, 85%, 90%, 91%, 92%, 93%, 94%, 95%, 96%, 97%, 98%, or 99% sequence identity to at: least seven contiguous amino acids of SEQ ID NO: 6311. In some embodiments, a cysteine-containing polypeptide comprises an isolated and purified polypeptide comprising at least 70% sequence identity to at least seven contiguous amino acids of SEQ ID NO: 6311. In some embodiments, a cysteine-containing polypeptide comprises an isolated and purified polypeptide comprising at least 75% sequence identity to at least seven contiguous amino acids of SEQ ID NO: 6311. In some embodiments, a cysteine-containing polypeptide comprises an isolated and purified polypeptide comprising at least 80% sequence identity to at least seven contiguous amino acids of SEQ ID NO: 6311. In some embodiments, a cysteine-containing polypeptide comprises an isolated and purified polypeptide comprising at least 85% sequence identity to at least seven contiguous amino acids of SEQ ID NO: 6311. In some embodiments, a cysteine-containing polypeptide comprises an isolated and purified polypeptide comprising at least 90% sequence identity to at least seven contiguous amino acids of SEQ ID NO: 6311. In some embodiments, a cysteine-containing polypeptide comprises an isolated and purified polypeptide comprising at least 91% sequence identity to at least seven contiguous amino acids of SEQ ID NO: 6311. In some embodiments, a cysteine-containing polypeptide comprises an isolated and purified polypeptide comprising at least 92% sequence identity to at least seven contiguous amino acids of SEQ ID NO: 6311. In some embodiments, a cysteine-containing polypeptide comprises an isolated and purified polypeptide comprising at least 93% sequence identity to at least seven contiguous amino acids of SEQ ID NO: 6311. In some embodiments, a cysteine-containing polypeptide comprises an isolated and purified polypeptide comprising at least 94% sequence identity to at least seven contiguous amino acids of SEQ ID NO: 6311. In some embodiments, a cysteine-containing polypeptide comprises an isolated and purified polypeptide comprising at least 95% sequence identity to at least seven contiguous amino acids of SEQ ID NO: 6311. In some embodiments, a cysteine-containing polypeptide comprises an isolated and purified polypeptide comprising at least 96% sequence identity to at least seven contiguous amino acids of SEQ ID NO: 6311. In some embodiments, a cysteine-containing polypeptide comprises an isolated and purified polypeptide comprising at least 97% sequence identity to at least seven contiguous amino acids of SEQ ID NO: 6311. In some embodiments, a cysteine-containing polypeptide comprises an isolated and purified polypeptide comprising at least 98% sequence identity to at least seven contiguous amino acids of SEQ ID NO: 6311. In some embodiments, a cysteine-containing polypeptide comprises an isolated and purified polypeptide comprising at least 99% sequence identity to at least seven contiguous amino acids of SEQ ID NO: 6311. In some embodiments, a cysteine-containing polypeptide comprises an isolated and purified polypeptide comprising 100% sequence identity to at least seven contiguous amino acids of SEQ ID NO: 6311.

In some embodiments, a cysteine-containing polypeptide comprises an isolated and purified polypeptide comprising at least 70%, 75%, 80%, 85%, 90%, 91%, 92%, 93%, 94%, 95%, 96%, 97%, 98%, or 99% sequence identity to SEQ ID NO: 6311. In some embodiments, a cysteine-containing polypeptide comprises an isolated and purified polypeptide comprising 100% sequence identity to SEQ ID NO: 6311. In some embodiments, a cysteine-containing polypeptide comprises an isolated and purified polypeptide consisting of 100% sequence identity to SEQ ID NO: 6311.

As used herein, a polypeptide includes natural amino acids, unnatural amino acids, or a combination thereof. In some instances, an amino acid residue refers to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

“Naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y, and V.

“Hydrophobic amino acids” include small hydrophobic amino acids and large hydrophobic amino acids. “Small hydrophobic amino acid” are glycine, alanine, proline, and analogs thereof. “Large hydrophobic amino acids” are valine, leucine, isoleucine, phenylalanine, methionine, tryptophan, and analogs thereof. “Polar amino acids” are serine, threonine, asparagine, glutamine, cysteine, tyrosine, and analogs thereof. “Charged amino acids” are lysine, arginine, histidine, aspartate, glutamate, and analogs thereof.

The term “amino acid analog” refers to a molecule which is structurally similar to an amino acid and which is substituted for an amino acid in the formation of a peptidomimetic macrocycle. Amino acid analogs include, without limitation, p-amino acids and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y, and V.

In some instances, amino acid analogs include 1-amino acid analogs. Examples of β-amino acid analogs include, but are not limited to, the following: cyclic p-amino acid analogs; 3-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-3-phenylalanine; 3-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-O-homoserine; O-benzyl-L-β-homothreonine; O-benzyl-L-β-homotyrosine; γ-trityl-L-O-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; No-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

In some instances, amino acid analogs include analogs of alanine, valine, glycine, or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

In some instances, amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; NS-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-omithine; D-ornithine; L-ornithine; Arg(Me)(Pbf)-OH; Arg(Me)₂-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

In some instances, amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

In some instances, amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cysteine, cystathionine, Cys(StBu)-OH, and acetamidomethyl-D-penicillamine.

In some instances, amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

In some instances, amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

In some instances, amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

In some instances, amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some instances, amino acid analogs are racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

### Cysteine-Containing Polypeptide Production

In some embodiments, a cysteine-containing polypeptide described above is generated recombinantly or is synthesized chemically. In some instances, a cysteine-containing polypeptide described above is generated recombinantly, for example, by a host cell system or in a cell-free system. In some instances, a cysteine-containing polypeptide described above is synthesized chemically.

In some embodiments, a cysteine-containing polypeptide described above is generated recombinantly by a host cell system. Exemplary host cell systems include a eukaryotic cell system (e.g., mammalian cell, insect cell, yeast cell or plant cell) or a prokaryotic cell system (e.g., gram-positive bacterium or a gram-negative bacterium).

In some embodiments, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris yeast strains such as GS 115, KM71H, SMD1168, SMD1168H, and X-33, and Saccharomyces cerevisiae yeast strains such as INVSc1.

In some embodiments, a eukaryotic host cell is a plant host cell. In some instances, the plant cells comprises a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable vectors for the production of a cysteine-containing polypeptide include any suitable vectors derived from either a eukaryotic or prokaryotic sources. Exemplary vectors include vectors from bacteria (e.g., E. coli), insects, yeast (e.g., Pichia pastoris), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is used for the production of a cysteine-containing polypeptide. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, or HeLa cells. Exemplary cell-free systems include E. coli S30 Extract system, E. coli T7 S30 system, or PURExpress®.

### Methods of Use

In some embodiments, disclosed herein include methods of modulating an immune response in a subject. In some embodiments, disclosed herein is a method of modulating an immune response in a subject, which comprises administering to the subject a therapeutically effective amount of a small molecule fragment of Formula (I):

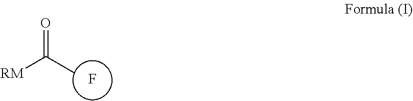

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with an endogenous cysteine-containing polypeptide expressed in the subject to form a cysteine-containing polypeptide-small molecule fragment adduct. In some instances, the cysteine-containing polypeptide-small molecule fragment adduct comprises a covalent bonding. In some cases, the cysteine-containing polypeptide-small molecule fragment adduct comprises an irreversible bonding. In other cases, the cysteine-containing polypeptide-small molecule fragment adduct comprises a reversible bonding. In some instances, an endogenous cysteine-containing polypeptide is a polypeptide that is expressed or present in a cell of interest (e.g., a diseased cell such as a cancerous cell). In some instances, an endogenous cysteine-containing polypeptide is a polypeptide that is overexpressed in a cell of interest (e.g., a diseased cell such as a cancerous cell). In some instances, an endogenous cysteine-containing polypeptide is a polypeptide that harbors one or more mutations in a cell of interest (e.g., a diseased cell such as a cancerous cell). In some instances, a mutation comprises a missense mutation, an insertion, or a deletion. In some instances, a mutation comprises a truncation, for example, a truncation at the N-terminus or the C-terminus of the polypeptide. In additional instances, an endogenous cysteine-containing polypeptide is a polypeptide that has an altered conformation in a cell of interest (e.g., a diseased cell such as a cancerous cell) relative to the conformation of the wild-type polypeptide.

In some instances, a cysteine-containing polypeptide-small molecule fragment adduct induces an immune response. In some cases, the immune response is a humoral immune response. In other cases, the immune response is a cell-mediated immune response. In some instances, the cysteine-containing polypeptide-small molecule fragment adduct induces a humoral immune response. In some instances, a cysteine-containing polypeptide-small molecule fragment adduct induces a cell-mediated immune response. In additional instances, a cysteine-containing polypeptide-small molecule fragment adduct induces a humoral immune response and a cell-mediated immune response. In some instances, humoral immunity (or antibody-mediated beta cellularis immune system) is the production of antibody and its accessory processes such as Th2 activation, cytokine production, germinal center formation, isotype switching, affinity maturation, and memory cell generation. In some instances, humoral immunity is mediated by macromolecules in the extracellular fluids. In some cases, cell-mediated immunity comprises activation of phagocytes, antigen-specific cytotoxic T-lymphocytes, and release of cytokines in response to an antigen. In some cases, cell-mediated immunity differs from humoral immunity in that it does not involve production of antibody.

In some embodiments, a cysteine-containing polypeptide-small molecule fragment adduct increases an immune response relative to a control. In some cases, a cysteine-containing polypeptide-small molecule fragment adduct increases a humoral immune response relative to a control. In additional cases, a cysteine-containing polypeptide-small molecule fragment adduct increases a cell-mediated immune response relative to a control. In additional cases, a cysteine-containing polypeptide-small molecule fragment adduct increases a humoral immune response and a cell-mediated immune response relative to a control.

In some cases, a control is the level of an immune response in the subject prior to administration of the small molecule fragment or is the level of an immune response in a subject who has not been exposed to the small molecule fragment. In some cases, a control is the level of a humoral immune response or a cell-mediated immune response in the subject prior to administration of the small molecule fragment or is the level of a humoral immune response or a cell-mediated immune response in a subject who has not been exposed to the small molecule fragment.

In some instances, a cysteine-containing polypeptide-small molecule fragment adduct modulates an immune response. In some cases, the immune response is a humoral immune response. In other cases, the immune response is a cell-mediated immune response. In some instances, the cysteine-containing polypeptide-small molecule fragment adduct modulates a humoral immune response. In some instances, a cysteine-containing polypeptide-small molecule fragment adduct modulates a cell-mediated immune response. In additional instances, a cysteine-containing polypeptide-small molecule fragment adduct modulates a humoral immune response and a cell-mediated immune response.

In some instances, a cysteine-containing polypeptide is a non-denatured form of the polypeptide.

In some instances, a cysteine-containing polypeptide comprises a biologically active cysteine site. As described above and elsewhere herein, in some cases, a biologically active cysteine site is a cysteine residue that is located about 10 Å or less to an active-site ligand or residue. In other cases, a biologically active cysteine site is a cysteine residue that is located greater than 10 Å from an active-site ligand or residue. In some cases, the cysteine residue that is located greater than 10 Å from the active-site ligand or residue is a non-active site cysteine.

Further as described elsewhere herein, a cysteine-containing polypeptide comprises, in some instances, an enzyme, a transporter, a receptor, a channel protein, an adaptor protein, a chaperone, a signaling protein, a plasma protein, transcription related protein, translation related protein, mitochondrial protein, or cytoskeleton related protein. In some cases, the cysteine-containing polypeptide comprises an enzyme, a transporter, a receptor, a channel protein, an adaptor protein, a chaperone, a signaling protein, transcription related protein, or translation related protein.

In some embodiments, a cysteine-containing polypeptide is about 20, 30, 35, 40, 45, 50, 55, 60, 65, 70, 75, 80, 85, 90, 95, 100, 150, 200, 250, 300, 350, 400, 450, 500, 600, 700, 800, 900, 1000, 1100, 1200, 1500, 2000, 2100, 2200, 2500 amino acid residues in length or more. In some cases, a cysteine-containing polypeptide is about 20 amino acid residues in length or more. In some cases, a cysteine-containing polypeptide is about 60 amino acid residues in length or more. In some cases, a cysteine-containing polypeptide is about 70 amino acid residues in length or more. In some cases, a cysteine-containing polypeptide is about 80 amino acid residues in length or more. In some cases, a cysteine-containing polypeptide is about 90 amino acid residues in length or more. In some cases, a cysteine-containing polypeptide is about 100 amino acid residues in length or more. In some cases, a cysteine-containing polypeptide is about 150 amino acid residues in length or more. In some cases, a cysteine-containing polypeptide is about 200 amino acid residues in length or more. In some cases, a cysteine-containing polypeptide is about 300 amino acid residues in length or more. In some cases, a cysteine-containing polypeptide is about 400 amino acid residues in length or more. In some cases, a cysteine-containing polypeptide is about 500 amino acid residues in length or more. In some cases, a cysteine-containing polypeptide is about 800 amino acid residues in length or more. In some cases, a cysteine-containing polypeptide is about 1000 amino acid residues in length or more. In some cases, a cysteine-containing polypeptide is about 1500 amino acid residues in length or more.

In some instances, a cysteine-containing polypeptide comprises a protein illustrated in Tables 1-5. In some cases, a cysteine-containing polypeptide comprises a protein illustrated in Table 1. In some cases, a cysteine-containing polypeptide comprises a protein illustrated in Table 2. In some cases, a cysteine-containing polypeptide comprises a protein illustrated in Table 3. In some cases, a cysteine-containing polypeptide comprises a protein illustrated in Table 4. In some cases, a cysteine-containing polypeptide comprises a protein illustrated in Table 5. In some instances, the cysteine residue of interest is denoted by a (*) in Tables 1-5.

In some instances, a cysteine-containing polypeptide comprises a protein illustrated in Tables 6A-6E. In some cases, a cysteine-containing polypeptide comprises a protein illustrated in Table 6A. In some cases, a cysteine-containing polypeptide comprises a protein illustrated in Table 6B. In some cases, a cysteine-containing polypeptide comprises a protein illustrated in Table 6C. In some cases, a cysteine-containing polypeptide comprises a protein illustrated in Table 6D. In some cases, a cysteine-containing polypeptide comprises a protein illustrated in Table 6E.

In some embodiments, as described above, a small molecule fragment comprises a Michael acceptor moiety which comprises an alkene or an alkyne moiety. In some instances, a covalent bond is formed between a portion of the Michael acceptor moiety on the small molecule fragment and a portion of a cysteine residue of the cysteine-containing polypeptide.

In some instances, a small molecule fragment comprises a small molecule fragment moiety F which is obtained from a compound library. In some instances, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some cases, F is a small molecule fragment moiety illustrated in FIG. 1 (e.g., FIG. 1A and/or FIG. 1B). In some cases, the small molecule fragment is a small molecule fragment illustrated in FIG. 1 (e.g., FIG. 1A and/or FIG. 1B).

In some instances, a small molecule fragment has a molecular weight of about 150 Dalton or higher. In some cases, a small molecular fragment has a molecular weight of about 175, 200, 225, 250, 275, 300, 350, 400, 450, 500, 550, 600, 650, 700, 750, 800, 850, 900, 950, 1000 Dalton, or higher. In some cases, a molecular weight of the small molecular fragment is prior to enrichment with a halogen, a nonmetal, or a transition metal. In some cases, a small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher.

In some instances, the method further comprises administration of a cysteine-containing polypeptide-small molecule fragment adduct. In some instances, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQ ID NOs: 1-9655. In some cases, the cysteine-containing polypeptide-small molecule fragment adduct further enhances or increases an immune response. In some instances, an enhancement or an increase of the immune response is relative to a level of the immune response prior to administration of the cysteine-containing polypeptide-small molecule fragment adduct.

In some embodiments, disclosed herein is a derivative of cereblon protein having the structure of Formula (I),

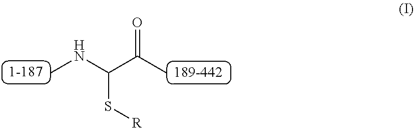

wherein,
   the derivation occurs at cereblon cysteine residue position 188 based on SEQ ID NO: 9665;
   R is selected from:

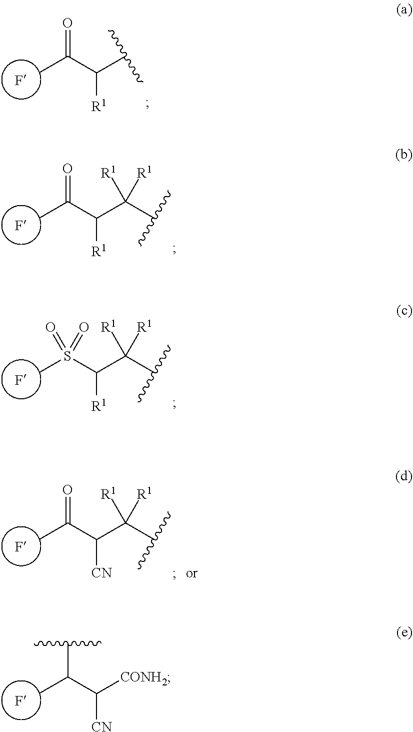

wherein R¹ is H, C1-C3 alkyl, or aryl; and
F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a derivative of cereblon protein having the structure of Formula (I),

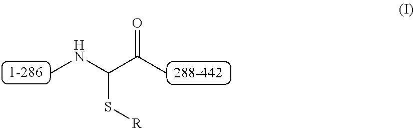

wherein,
   the derivation occurs at cereblon cysteine residue position 287 based on SEQ ID NO: 9665;
   R is selected from:

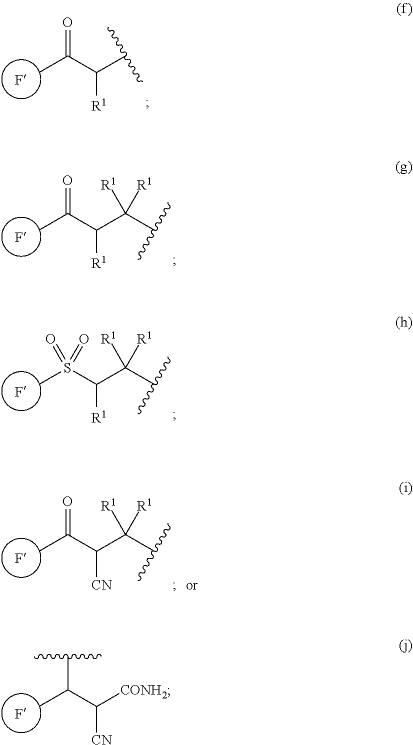

wherein R¹ is H, C1-C3 alkyl, or aryl; and
F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some cases, the method further comprises administration of an adjuvant.

In some cases, the small molecule fragment is formulated for parenteral, oral, or intranasal administration.

### Diseases or Indications

In some embodiments, disclosed herein include a method of administrating a small molecule fragment to a subject in which the small molecule fragment interacts with an endogenous cysteine-containing polypeptide expressed in the subject to form a cysteine-containing polypeptide-small molecule fragment adduct. In some embodiments, the cysteine-containing polypeptide is overexpressed in a disease or condition. In some cases, the overexpressed cysteine-containing polypeptide comprises one or more mutations. In some cases, the cysteine-containing polypeptide comprising one or more mutations is overexpressed in a disease or condition.

In some instances, the disease or condition is cancer. In some cases, the cysteine-containing polypeptide is a cancer-associated protein. In some cases, the cysteine-containing polypeptide is overexpressed in a cancer. In some cases, the cysteine-containing polypeptide comprising one or more mutations is overexpressed in a cancer. In some instances, a mutation comprises a missense mutation, an insertion, or a deletion. In some instances, a mutation comprises a truncation at a terminus of a protein. In some instances, a mutation alters the conformation of a protein relative to the conformation of its wild-type protein. In additional instances, a mutation does not alter the conformation of a protein.

In some instances, a cancer is a solid tumor. In some instances, a cancer is a hematologic malignancy. In some instances, a cancer is a relapsed or refractory cancer, or a metastatic cancer. In some instances, a solid tumor is a relapsed or refractory solid tumor, or a metastatic solid tumor. In some cases, a hematologic malignancy is a relapsed or refractory hematologic malignancy, or a metastatic hematologic malignancy.

In some embodiments, a cancer is a solid tumor. Exemplary solid tumor includes, but is not limited to, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer or vulvar cancer.

In some embodiments, a cysteine-containing polypeptide described herein that is overexpressed and/or comprises one or more mutations is present in a solid tumor. In some cases, a cysteine-containing polypeptide described herein that is overexpressed and/or comprises one or more mutations is present in metastatic solid tumor. In some cases, a cysteine-containing polypeptide described herein that is overexpressed and/or comprises one or more mutations is present in a relapsed or refractory solid tumor. In some instances, a small molecule fragment described herein interacts with a cysteine-containing polypeptide that is present, overexpressed, and/or comprises a mutation in a solid tumor.

In some instances, a cancer is a hematologic malignancy. In some instances, a hematologic malignancy is a leukemia, a lymphoma, a myeloma, a non-Hodgkin's lymphoma, or a Hodgkin's lymphoma. In some instances, a hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, a cysteine-containing polypeptide described herein that is overexpressed and/or comprises one or more mutations is present in a hematologic malignancy. In some cases, a cysteine-containing polypeptide described herein that is overexpressed and/or comprises one or more mutations is present in metastatic hematologic malignancy. In some cases, a cysteine-containing polypeptide described herein that is overexpressed and/or comprises one or more mutations is present in a relapsed or refractory hematologic malignancy. In some instances, a small molecule fragment described herein interacts with a cysteine-containing polypeptide that is present, overexpressed, and/or comprises a mutation in a hematologic malignancy.

### Vaccines

In some embodiments, disclosed herein include vaccines and vaccine formulations that comprises a small molecule fragment described herein, an antibody that recognizes a cysteine-containing polypeptide-small molecule fragment adduct described herein, or a cysteine-containing polypeptide-small molecule fragment adduct described herein. In some embodiments, disclosed herein is a vaccine that comprises a small molecule fragment described herein. In some embodiments, disclosed herein is a vaccine that comprises an antibody that recognizes a cysteine-containing polypeptide-small molecule fragment adduct described herein. In some embodiments, disclosed herein is a vaccine that comprises a cysteine-containing polypeptide-small molecule fragment adduct described herein.

In some instances, a cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some cases, the cysteine-containing polypeptide is an enzyme, a transporter, a receptor, a channel protein, an adaptor protein, a chaperone, a signaling protein, transcription related protein, or translation related protein.

In some instances, a cysteine-containing polypeptide comprises a protein illustrated in Tables 1-6. In some cases, a cysteine-containing polypeptide comprises a protein illustrated in Table 1. In some cases, a cysteine-containing polypeptide comprises a protein illustrated in Table 2. In some cases, a cysteine-containing polypeptide comprises a protein illustrated in Table 3. In some cases, a cysteine-containing polypeptide comprises a protein illustrated in Table 4. In some cases, a cysteine-containing polypeptide comprises a protein illustrated in Table 5. In some cases, a cysteine-containing polypeptide comprises a protein illustrated in Table 6 (e.g., Tables 6A-6E).

In some embodiments, a small molecule fragment comprises a Michael acceptor moiety which comprises an alkene or an alkyne moiety. In some instances, a covalent bond is formed between a portion of the Michael acceptor moiety on the small molecule fragment and a portion of a cysteine residue of the cysteine-containing polypeptide.

In some instances, a small molecule fragment comprises a small molecule fragment moiety F which is obtained from a compound library. In some instances, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some cases, F is a small molecule fragment moiety illustrated in FIG. 1 (e.g., FIG. 1A and/or FIG. 1B). In some cases, the small molecule fragment is a small molecule fragment illustrated in FIG. 1 (e.g., FIG. 1A and/or FIG. 1B).

In some instances, a small molecule fragment has a molecular weight of about 150 Dalton or higher. In some cases, a small molecular fragment has a molecular weight of about 175, 200, 225, 250, 275, 300, 350, 400, 450, 500, 550, 600, 650, 700, 750, 800, 850, 900, 950, 1000 Dalton, or higher. In some cases, the molecular weight of the small molecular fragment is prior to enrichment with a halogen, a nonmetal, or a transition metal. In some cases, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher.

In some instances, a vaccine is formulated in a conventional manner using one or more physiologically acceptable carriers including excipients and auxiliaries which facilitate processing of the active agents into preparations which are used pharmaceutically. Proper formulation is dependent upon the route of administration chosen. Any of the well-known techniques, carriers, and excipients are used as suitable and as understood in the art.

In some instances, a vaccine is further formulated with a cysteine-containing polypeptide-small molecule fragment adduct. In some instances, a cysteine-containing polypeptide-small molecule fragment adduct enhances an immune response. In some instances, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQ ID NOs: 1-9655.

In some embodiments, disclosed herein is a vaccine comprising an antibody or its binding fragment thereof that recognizes a derivative of a cysteine-containing protein having the structure of Formula (I),

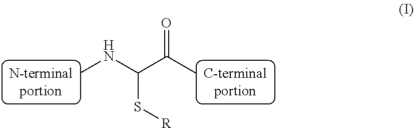

wherein,
   the derivation occurs at a cysteine residue;
   R is selected from:

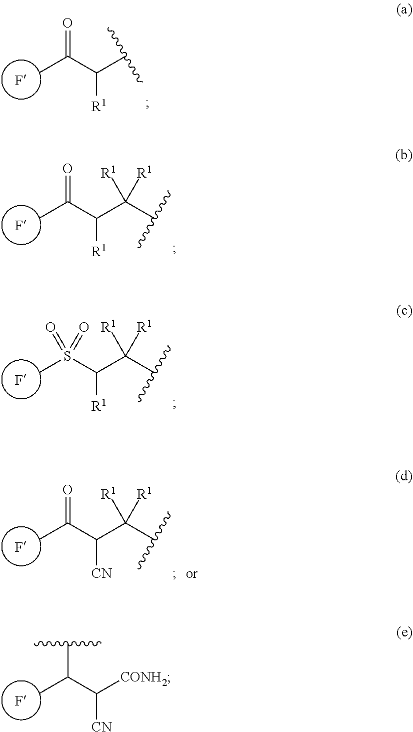

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of IDH1 protein having the structure of Formula (I),

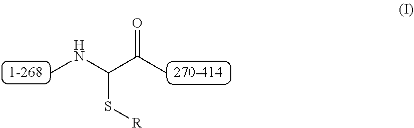

wherein,
   the derivation occurs at IDH1 cysteine residue position 269 based on SEQ ID NO: 9656;
   R is selected from:

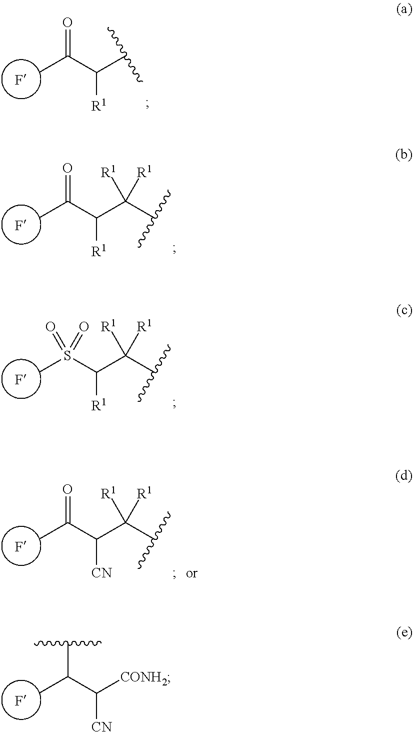

wherein R¹ is H, C1-C3 alkyl, or aryl; and
F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of IDH2 protein having the structure of Formula (I),

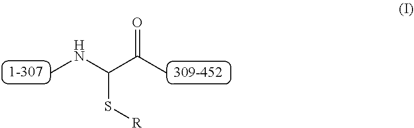

wherein,
the derivation occurs at IDH2 cysteine residue position 308 based on SEQ ID NO: 9657;
R is selected from:

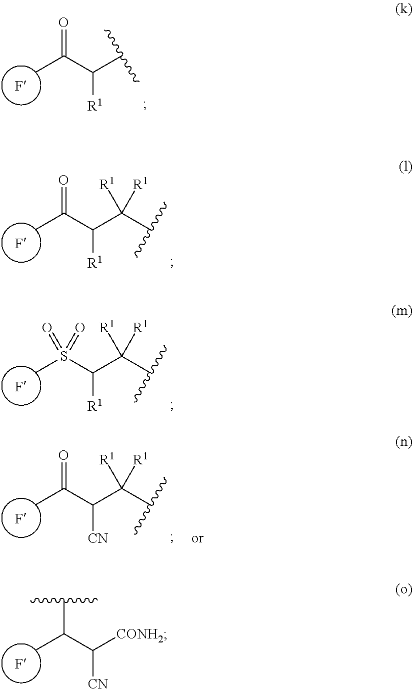

wherein R is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of caspase-8 protein having the structure of Formula (I),

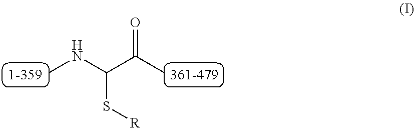

wherein,
the derivation occurs at caspase-8 cysteine residue position 360 based on SEQ ID NO: 9658;
R is selected from:

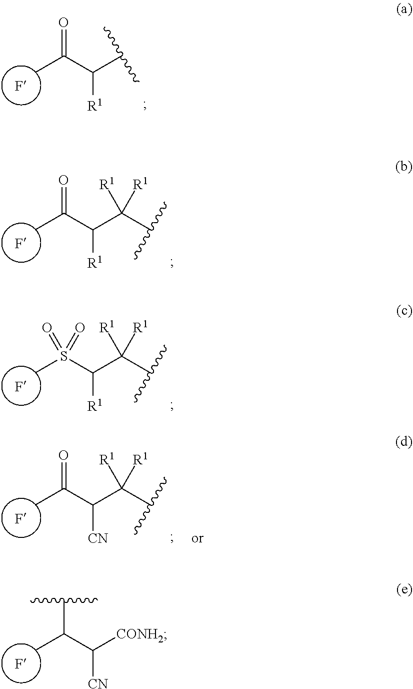

wherein R is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of caspase-10 protein having the structure of Formula (I),

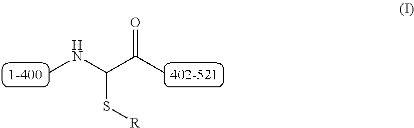

wherein,
the derivation occurs at caspase-10 cysteine residue position 401 based on SEQ ID NO: 9659;
R is selected from:

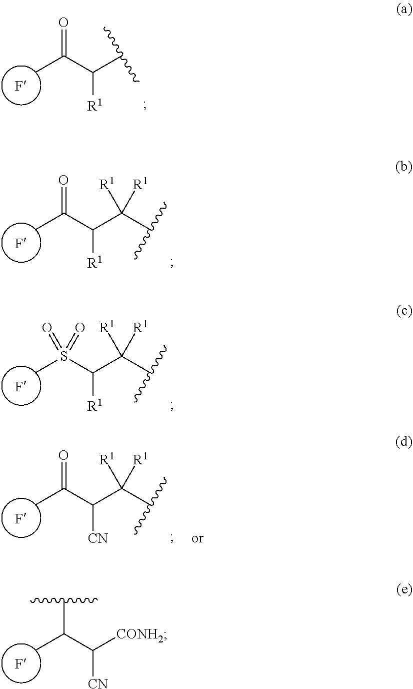

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of PRMT-1 protein having the structure of Formula (I),

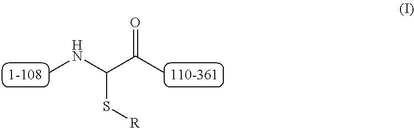

wherein,
the derivation occurs at PRMT-1 cysteine residue position 109 based on SEQ ID NO: 9660;
R is selected from:

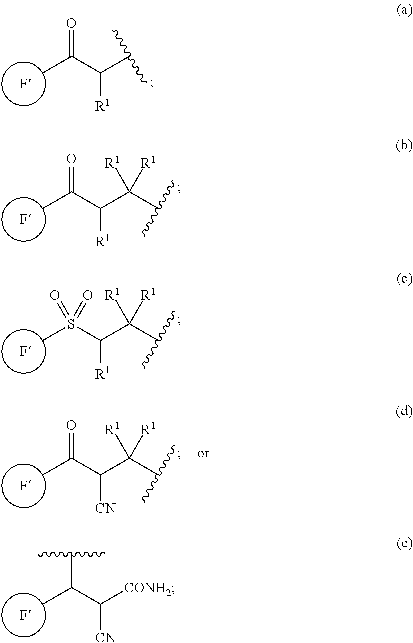

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of ZAK protein having the structure of Formula (I),

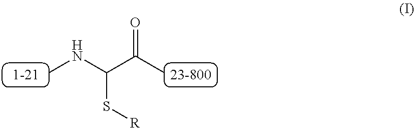

wherein,
the derivation occurs at ZAK cysteine residue position 22 based on SEQ ID NO: 9661;
R is selected from:

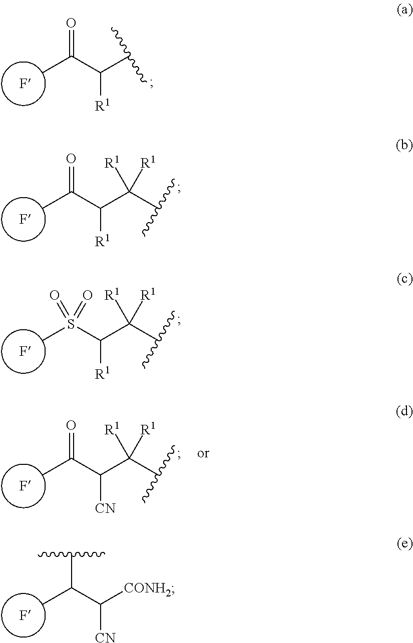

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of IMPDH2 protein having the structure of Formula (I),

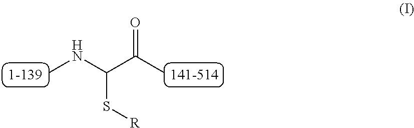

wherein,
the derivation occurs at IMPDH2 cysteine residue position 140 based on SEQ ID NO: 9662;
R is selected from:

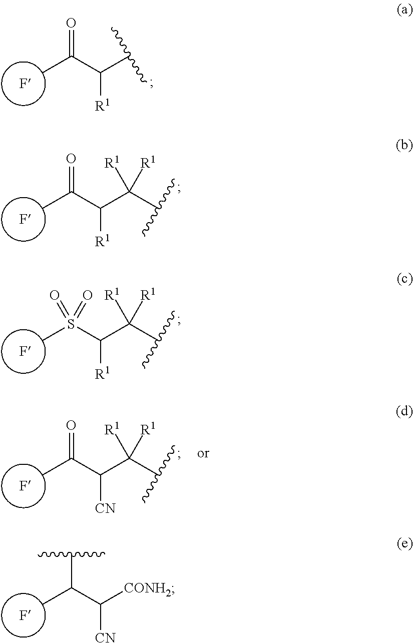

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of IMPDH2 protein having the structure of Formula (I),

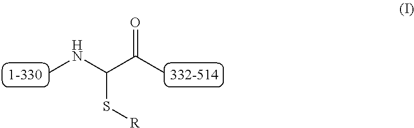

wherein,
the derivation occurs at IMPDH2 cysteine residue position 331 based on SEQ ID NO: 9662;
R is selected from:

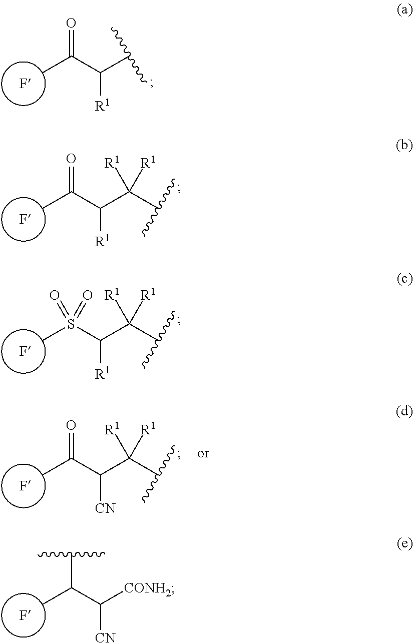

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of TIGAR protein having the structure of Formula (I),

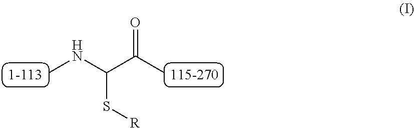

wherein,
the derivation occurs at TIGAR cysteine residue position 114 based on SEQ ID NO: 9663;
R is selected from:

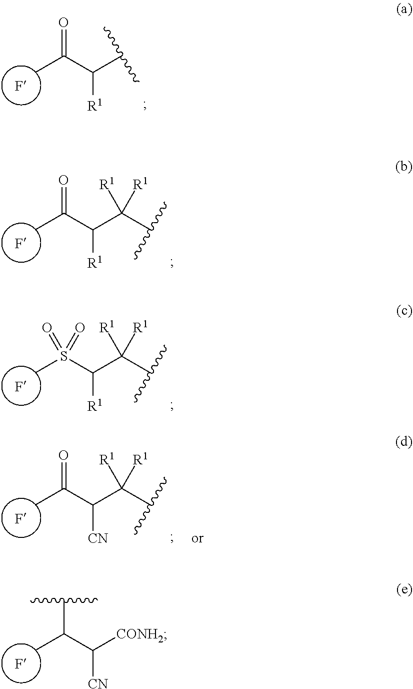

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of TIGAR protein having the structure of Formula (I),

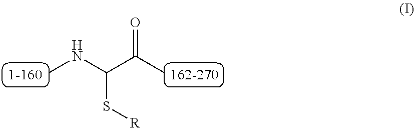

wherein,
   the derivation occurs at TIGAR cysteine residue position 161 based on SEQ ID NO: 9663;
   R is selected from:

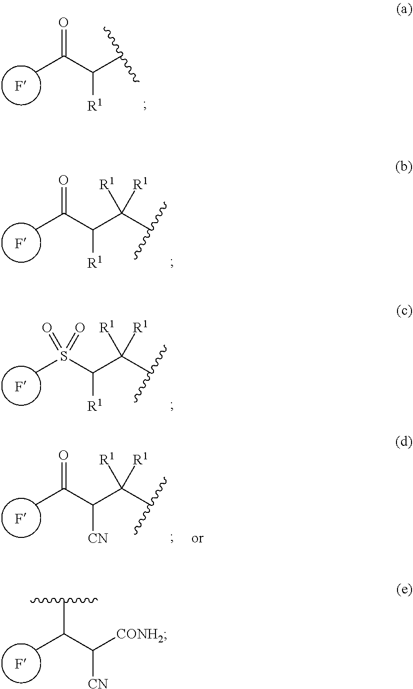

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of PKCθ protein having the structure of Formula (I),

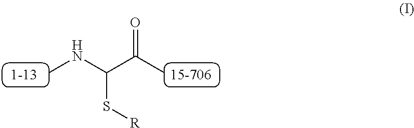

wherein,
the derivation occurs at PKCθ cysteine residue position 14 based on SEQ ID NO: 9664;
R is selected from:

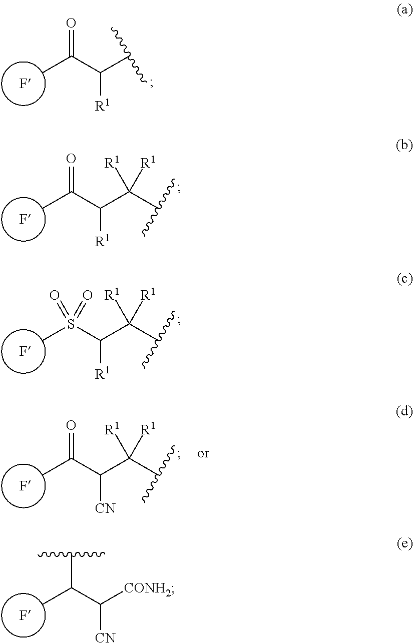

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of PKCθ protein having the structure of Formula (I),

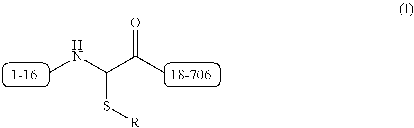

wherein,
the derivation occurs at PKCθ cysteine residue position 17 based on SEQ ID NO: 9664;
R is selected from:

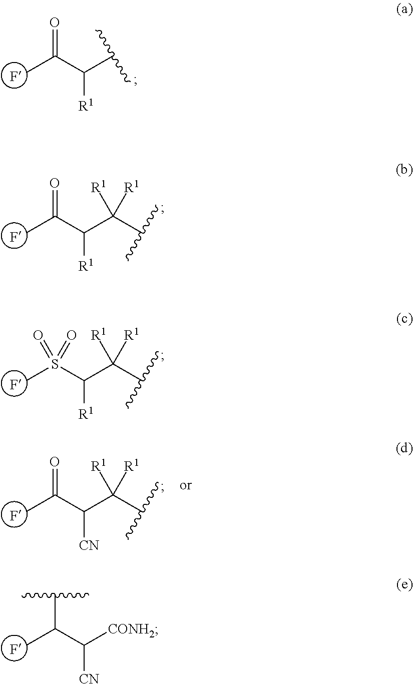

wherein R¹ is H, C1-C3 alkyl, or aryl; and

F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of cereblon protein having the structure of Formula (I),

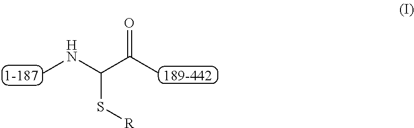

wherein,
   the derivation occurs at cereblon cysteine residue position 188 based on SEQ ID NO: 9665;
   R is selected from:

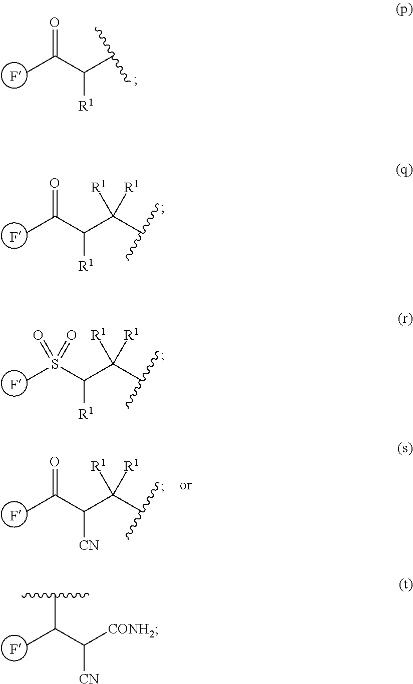

wherein R¹ is H, C1-C3 alkyl, or aryl; and
F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of cereblon protein having the structure of Formula (I),

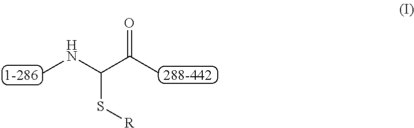

wherein,
   the derivation occurs at cereblon cysteine residue position 287 based on SEQ ID NO: 9665;
   R is selected from:

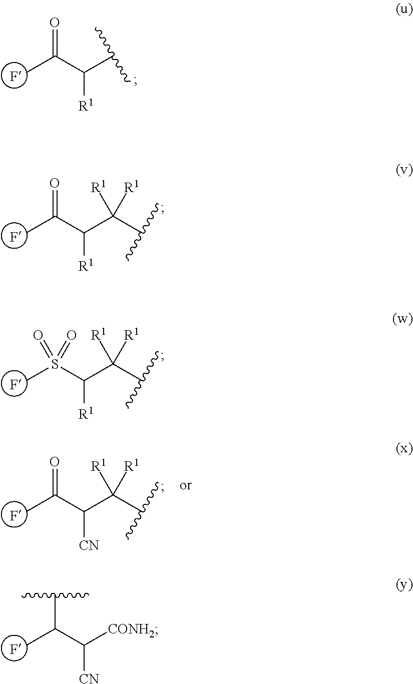

wherein R¹ is H, C1-C3 alkyl, or aryl; and
F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in FIG. 1 (e.g., FIG. 1A and/or FIG. 1B).

In some embodiments, disclosed herein is a vaccine comprising an antibody or its binding fragment thereof that recognizes a derivative of a cysteine containing protein that comprises the motif XₚC*Z, wherein Xₚ is a polar residue, C* denotes the site of modification, and Z is any amino acid. In some cases, the cysteine containing protein is selected from AIP, PES1, IKBKB, XPO1, KDM4B, NR3C1, GSTP1, TNFAIP3, ACAT1, IRAK1, GNB2L1, IRF4, USP34, ZC3HAV1, USP7, PELI1, DCUN1D1, USP28, UBE2O, RRAGC, MLTK, USP22, KDM3A, and USP16.

In some embodiments, disclosed herein is a vaccine comprising an antibody or its binding fragment thereof that recognizes a derivative of a cysteine containing protein that comprises the motif XₚC*Xₙ, wherein Xₚ is a polar residue, C* denotes the site of modification, and Xₙ is a nonpolar residue. In some cases, the cysteine containing protein is selected from AIP, PES1, IKBKB, XPO1, GSTP1, ACAT1, IRAK1, IRF4, ZC3HAV1, USP7, PELI1, USP28, UBE20, RRAGC, MLTK, USP22, KDM3A, and USP16.

In some embodiments, disclosed herein is a vaccine comprising an antibody or its binding fragment thereof that recognizes a derivative of a cysteine containing protein that comprises the motif XₚC*Xₚ, wherein Xₚ is a polar residue and C* denotes the site of modification. In some cases, the cysteine containing protein is selected from KDM4B, NR3C1, TNFAIP3, USP7, and USP22.

In some embodiments, disclosed herein is a vaccine comprising an antibody or its binding fragment thereof that recognizes a derivative of a cysteine containing protein that comprises the motif XₚC*X_{b}, wherein Xₚ is a polar residue, C* denotes the site of modification, and X_{b} is a basic residue. In some cases, the cysteine containing protein is selected from GNB2L1 and USP34.

In some embodiments, disclosed herein is a vaccine comprising an antibody or its binding fragment thereof that recognizes a derivative of a cysteine containing protein that comprises the motif XₚC*Xₐ, wherein Xₚ is a polar residue, C* denotes the site of modification, and Xₐ is an acidic residue. In some cases, the cysteine containing protein is DCUN1D1.

In some embodiments, disclosed herein is a vaccine comprising an antibody or its binding fragment thereof that recognizes a derivative of a cysteine containing protein that comprises the motif SC*Z, wherein C* denotes the site of modification, and Z is any amino acid. In some cases, the cysteine containing protein is selected from PES1, IKBKB, GSTP1, ACAT1, IRAK1, ZC3HAV1, and RRAGC.

In some embodiments, disclosed herein is a vaccine comprising an antibody or its binding fragment thereof that recognizes a derivative of a cysteine containing protein that comprises the motif NC*Z, wherein C* denotes the site of modification, and Z is any amino acid. In some cases, the cysteine containing protein is selected from XPO1, GNB2L1, USP34, UBE2O, MLTK, and USP22.

In some embodiments, disclosed herein is a vaccine comprising an antibody or its binding fragment thereof that recognizes a derivative of a cysteine containing protein that comprises the motif YC*Z, wherein C* denotes the site of modification, and Z is any amino acid. In some cases, the cysteine containing protein is selected from KDM4B and NR3C1.

In some embodiments, disclosed herein is a vaccine comprising an antibody or its binding fragment thereof that recognizes a derivative of a cysteine containing protein that comprises the motif TC*Z, wherein C* denotes the site of modification, and Z is any amino acid. In some cases, the cysteine containing protein is selected from TNFAIP3, USP7, USP28, KDM3A, and USP16.

In some embodiments, disclosed herein is a vaccine comprising an antibody or its binding fragment thereof that recognizes a derivative of a cysteine containing protein that comprises the motif QC*Z, wherein C* denotes the site of modification, and Z is any amino acid. In some cases, the cysteine containing protein is selected from IRF4, PELI1, DCUN1D1, and USP22.

In some embodiments, disclosed herein is a vaccine comprising an antibody or its binding fragment thereof that recognizes a derivative of a cysteine containing protein that comprises the motif CC*Z, wherein C* denotes the site of modification, and Z is any amino acid. In some cases, the cysteine containing protein is AIP.

In some embodiments, disclosed herein is a vaccine comprising an antibody or its binding fragment thereof that recognizes a derivative of a cysteine containing protein that comprises the motif XₚC*Z, wherein Xₚ is a polar residue, C* denotes the site of modification, and Z is any amino acid. In some cases, the cysteine containing protein is selected from IKBKB, KDM4B, GSTP1, TNFAIP3, ACAT1, IRAK1, USP34, USP7, PELI1, USP28, UBE2O, MLTK, USP22, KDM3A, and USP16.

In some embodiments, disclosed herein is a vaccine comprising an antibody or its binding fragment thereof that recognizes a derivative of a cysteine containing protein that comprises the motif XₚC*Z, wherein Xₚ is a polar residue, C* denotes the site of modification, and Z is any amino acid. In some cases, the cysteine containing protein is selected from NR3C1, IRF4, and ZC3HAV1.

In some embodiments, disclosed herein is a vaccine comprising an antibody or its binding fragment thereof that recognizes a derivative of a cysteine containing protein that comprises the motif XₚC*Z, wherein Xₚ is a polar residue, C* denotes the site of modification, and Z is any amino acid. In some cases, the cysteine containing protein is selected from GNB2L1 and RRAGC.

In some embodiments, disclosed herein is a vaccine comprising an antibody or its binding fragment thereof that recognizes a derivative of a cysteine containing protein that comprises the motif XₚC*Z, wherein Xₚ is a polar residue, C* denotes the site of modification, and Z is any amino acid. In some cases, the cysteine containing protein is selected from AIP, PES1, XPO1, and DCUN1D1.

In some embodiments, disclosed herein is a vaccine comprising an antibody or its binding fragment thereof that recognizes a derivative of a cysteine containing protein that comprises the motif XₙC*Z, wherein Xₙ is a nonpolar residue, C* denotes the site of modification, and Z is any amino acid. In some cases, the cysteine containing protein is selected from PES1, CYR61, UBE2L6, XPO1, ADA, NR3C1, POU2F2, UCHL3, MGMT, ERCC3, ACAT1, STAT3, UBA7, CASP2, IDH2, LRBA, UBE2L3, RELB, IRF8, CASP8, PDIA6, PCK2, PFKFB4, PDE12, USP34, USP48, SMARCC2, and SAMHD1.

In some embodiments, disclosed herein is a vaccine comprising an antibody or its binding fragment thereof that recognizes a derivative of a cysteine containing protein that comprises the motif XₙC*Xₙ, wherein Xₙ is a nonpolar residue and C* denotes the site of modification. In some cases, the cysteine containing protein is selected from PES1, CYR61, NR3C1, UCHL3, ERCC3, ACAT1, STAT3, CASP2, LRBA, UBE2L3, RELB, PDIA6, PCK2, PFKFB4, USP48, and SMARCC2.

In some embodiments, disclosed herein is a vaccine comprising an antibody or its binding fragment thereof that recognizes a derivative of a cysteine containing protein that comprises the motif XₙC*Xₚ, wherein Xₐ is a nonpolar residue, C* denotes the site of modification, and Xₚ is a polar residue. In some cases, the cysteine containing protein is selected from UBE2L6, POU2F2, MGMT, ACAT1, UBA7, CASP8, PDE12, and USP34.

In some embodiments, disclosed herein is a vaccine comprising an antibody or its binding fragment thereof that recognizes a derivative of a cysteine containing protein that comprises the motif XₙC*Xₐ, wherein Xₙ is a nonpolar residue, C* denotes the site of modification, and Xₐ is an acidic residue. In some cases, the cysteine containing protein is selected from CYR61 and XPO1.

In some embodiments, disclosed herein is a vaccine comprising an antibody or its binding fragment thereof that recognizes a derivative of a cysteine containing protein that comprises the motif XₙC*X_{b}, wherein Xₙ is a nonpolar residue, C* denotes the site of modification, and X_{b} is a basic residue. In some cases, the cysteine containing protein is selected from ADA, MGMT, IDH2, IRF8, and SAMHD1.

In some embodiments, disclosed herein is a vaccine comprising an antibody or its binding fragment thereof that recognizes a derivative of a cysteine containing protein that comprises the motif LC*Z, wherein C* denotes the site of modification, and Z is any amino acid. In some cases, the cysteine containing protein is selected from PES1, CYR61, XPO1, NR3C1, and SMARCC2.

In some embodiments, disclosed herein is a vaccine comprising an antibody or its binding fragment thereof that recognizes a derivative of a cysteine containing protein that comprises the motif PC*Z, wherein C* denotes the site of modification, and Z is any amino acid. In some cases, the cysteine containing protein is selected from CYR61, UBE2L6, MGMT, ERCC3, ACAT1, and USP48.

In some embodiments, disclosed herein is a vaccine comprising an antibody or its binding fragment thereof that recognizes a derivative of a cysteine containing protein that comprises the motif GC*Z, wherein C* denotes the site of modification, and Z is any amino acid. In some cases, the cysteine containing protein is selected from ADA, RELB, and USP34.

In some embodiments, disclosed herein is a vaccine comprising an antibody or its binding fragment thereof that recognizes a derivative of a cysteine containing protein that comprises the motif AC*Z, wherein C* denotes the site of modification, and Z is any amino acid. In some cases, the cysteine containing protein is selected from UCHL3, CASP2, IDH2, LRBA, CASP8, PCK2, and PDE12.

In some embodiments, disclosed herein is a vaccine comprising an antibody or its binding fragment thereof that recognizes a derivative of a cysteine containing protein that comprises the motif VC*Z, wherein C* denotes the site of modification, and Z is any amino acid. In some cases, the cysteine containing protein is selected from MGMT, ACAT1, UBA7, UBE2L3, and IRF8.

In some embodiments, disclosed herein is a vaccine comprising an antibody or its binding fragment thereof that recognizes a derivative of a cysteine containing protein that comprises the motif XᵣC*Z, wherein X, denotes an aromatic residue, C* denotes the site of modification, and Z is any amino acid. In some cases, the cysteine containing protein is selected from POU2F2, PDIA6, and SAMHD1.

In some embodiments, disclosed herein is a vaccine comprising an antibody or its binding fragment thereof that recognizes a derivative of a cysteine containing protein that comprises the motif XₐC*Z, wherein Xₙ is a nonpolar residue, C* denotes the site of modification, and Z is any amino acid. In some cases, the cysteine containing protein is selected from UBE2L6, ADA, UCHL3, MGMT, ERCC3, ACAT1, UBA7, CASP2, IDH2, UBE2L3, CASP8, PDIA6, PCK2, PFKFB4, PDE12, USP34, USP48, and SAMHD1.

In some embodiments, disclosed herein is a vaccine comprising an antibody or its binding fragment thereof that recognizes a derivative of a cysteine containing protein that comprises the motif XₙC*Z, wherein Xₙ is a nonpolar residue, C* denotes the site of modification, and Z is any amino acid. In some cases, the cysteine containing protein is selected from NR3C1, POU2F2, STAT3, RELB, IRF8, and SMARCC2.

In some embodiments, disclosed herein is a vaccine comprising an antibody or its binding fragment thereof that recognizes a derivative of a cysteine containing protein that comprises the motif XₙC*Z, wherein X, is a nonpolar residue, C* denotes the site of modification, and Z is any amino acid. In some cases, the cysteine containing protein is selected from PES1, CYR61, XPO1, and LRBA.

In some embodiments, disclosed herein is a vaccine comprising an antibody or its binding fragment thereof that recognizes a derivative of a cysteine containing protein that comprises the motif XₐC*Z, wherein Xₐ is an acidic residue, C* denotes the site of modification, and Z is any amino acid. In some cases, the cysteine containing protein is selected from ZAP70, PRKCQ, and PRMT1.

In some embodiments, disclosed herein is a vaccine comprising an antibody or its binding fragment thereof that recognizes a derivative of a cysteine containing protein that comprises the motif EC*Z, wherein C* denotes the site of modification, and Z is any amino acid. In some cases, the cysteine containing protein is selected from ZAP70 and PRKCQ.

In some embodiments, disclosed herein is a vaccine comprising an antibody or its binding fragment thereof that recognizes a derivative of a cysteine containing protein that comprises the motif X_{b}C*Z, wherein X_{b} is a basic residue, C* denotes the site of modification, and Z is any amino acid. In some cases, the cysteine containing protein is selected from CYR61, ZNF217, NCF1, IREB2, LRBA, CDK5, EP300, EZH2, UBE2S, VCPIP1, RRAGC, and IRAK4.

In some embodiments, disclosed herein is a vaccine comprising an antibody or its binding fragment thereof that recognizes a derivative of a cysteine containing protein that comprises the motif X_{b}C*Xₙ, wherein X_{b} is a basic residue, C* denotes the site of modification, and Xₙ is a nonpolar residue. In some cases, the cysteine containing protein is selected from CYR61, ZNF217, IREB2, EP300, UBE2S, VCPIP1, RRAGC, and IRAK4.

In some embodiments, disclosed herein is a vaccine comprising an antibody or its binding fragment thereof that recognizes a derivative of a cysteine containing protein that comprises the motif X_{b}C*Xₚ, wherein X_{b} is a basic residue, C* denotes the site of modification, and Xₚ is a polar residue. In some cases, the cysteine containing protein is selected from NCF1, LRBA, and CDK5.

In some embodiments, disclosed herein is a vaccine comprising an antibody or its binding fragment thereof that recognizes a derivative of a cysteine containing protein that comprises the motif X_{b}C*X_{b}, wherein X_{b} is a basic residue and C* denotes the site of modification. In some cases, the cysteine containing protein is EZH2.

In some embodiments, disclosed herein is a vaccine comprising an antibody or its binding fragment thereof that recognizes a derivative of a cysteine containing protein that comprises the motif RC*Z, wherein C* denotes the site of modification, and Z is any amino acid. In some cases, the cysteine containing protein is selected from ZNF217, NCF1, CDK5, EP300, and IRAK4.

In some embodiments, disclosed herein is a vaccine comprising an antibody or its binding fragment thereof that recognizes a derivative of a cysteine containing protein that comprises the motif KC*Z, wherein C* denotes the site of modification, and Z is any amino acid. In some cases, the cysteine containing protein is selected from CYR61, IREB2, LRBA, and UBE2S.

In some embodiments, disclosed herein is a vaccine comprising an antibody or its binding fragment thereof that recognizes a derivative of a cysteine containing protein that comprises the motif HC*Z, wherein C* denotes the site of modification, and Z is any amino acid. In some cases, the cysteine containing protein is selected from EZH2, VCPIP1, and RRAGC.

In some embodiments, disclosed herein is a vaccine comprising an antibody or its binding fragment thereof that recognizes a derivative of a cysteine containing protein that comprises the motif X_{b}C*Z, wherein X_{b} is a basic residue, C* denotes the site of modification, and Z is any amino acid. In some cases, the cysteine containing protein is selected from CDK5, EP300, EZH2, UBE2S, VCPIP1, and IRAK4.

In some embodiments, disclosed herein is a vaccine comprising an antibody or its binding fragment thereof that recognizes a derivative of a cysteine containing protein that comprises the motif X_{b}C*Z, wherein X_{b} is a basic residue, C* denotes the site of modification, and Z is any amino acid. In some cases, the cysteine containing protein is selected from ZNF217 and IREB2.

In some embodiments, disclosed herein is a vaccine comprising an antibody or its binding fragment thereof that recognizes a derivative of a cysteine containing protein that comprises the motif X_{b}C*Z, wherein X_{b} is a basic residue, C* denotes the site of modification, and Z is any amino acid. In some cases, the adapter, scaffolding protein or the modulator protein is selected from NCF1.

In some embodiments, disclosed herein is a vaccine comprising an antibody or its binding fragment thereof that recognizes a derivative of a cysteine containing protein that comprises the motif X_{b}C*Z, wherein X_{b} is a basic residue, C* denotes the site of modification, and Z is any amino acid. In some cases, the cysteine containing protein is selected from RRAGC.

In some embodiments, disclosed herein is a vaccine comprising an antibody or its binding fragment thereof that recognizes a derivative of a cysteine containing protein that comprises the motif X_{b}C*Z, wherein X_{b} is a basic residue, C* denotes the site of modification, and Z is any amino acid. In some cases, the cysteine containing protein is selected from CYR61 and LRBA.

In some cases, a vaccine described herein is further formulated with an adjuvant and/or additional carriers or excipients;

### Adjuvant

In some embodiments, the pharmaceutical composition and/or the vaccine further comprises an adjuvant. In some instances, an adjuvant enhances the immune response (humoral and/or cellular) elicited in a subject receiving the pharmaceutical composition and/or the vaccine. In some instances, an adjuvant elicits a Th1-type response. In other instances, an adjuvant elicits a Th2-type response. In some instances, a Th1-type response is characterized by the production of cytokines such as IFN-γ as opposed to a Th2-type response which is characterized by the production of cytokines such as IL-4, IL-5, and IL-10.

In some embodiments, an adjuvant comprises a stimulatory molecule such as a cytokine. Non-limiting examples of cytokines include: CCL20, a-interferon (IFN-a), β-interferon (IFN-β), γ-interferon, platelet derived growth factor (PDGF), TNFa, TNFp, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-1a, MIP-1-, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2.

Additional adjuvants include, for example: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some embodiments, an adjuvant is a modulator of a toll like receptor. Examples of modulators of toll-like receptors include TLR-9 agonists and are not limited to small molecule modulators of toll-like receptors such as Imiquimod. Other examples of adjuvants that are used in combination with a vaccine described herein include and are not limited to saponin, CpG ODN, and the like.

Sometimes, an adjuvant is selected from bacteria toxoids, polyoxypropylene-polyoxyethylene block polymers, aluminum salts, liposomes, CpG polymers, oil-in-water emulsions, or a combination thereof.

In some embodiments, an adjuvant is a lipid-based adjuvant, such as MPLA and MDP. In some instances, monophosphoryl lipid A (MPLA) is an adjuvant that causes increased presentation of liposomal antigen to specific T Lymphocytes. In some cases, a muramyl dipeptide (MDP) is used as a suitable adjuvant in conjunction with the vaccine formulations described herein.

In some embodiments, an adjuvant is an oil-in-water emulsion. The oil-in-water emulsion suitable for use with a vaccine described herein include, for example, at least one oil and at least one surfactant, with the oil(s) and surfactant(s) being biodegradable (metabolisable) and biocompatible. In some instances, the oil droplets in the emulsion is less than 5 μm in diameter, or have a sub-micron diameter, with these small sizes being achieved with a high pressure homogenizer to provide stable emulsions. Droplets with a size less than 220 nm are optionally subjected to filter sterilization.

In some instances, oils used include such as those from an animal (such as fish) or vegetable source. Sources for vegetable oils include, for example, nuts, seeds and grains. Peanut oil, soybean oil, coconut oil, and olive oil, exemplify the nut oils. Jojoba oil is used e.g. obtained from the jojoba bean. Seed oils include safflower oil, cottonseed oil, sunflower seed oil, sesame seed oil, etc. The grain group include: corn oil and oils of other cereal grains such as wheat, oats, rye, rice, teff, triticale, and the like. 6-10 carbon fatty acid esters of glycerol and 1,2-propanediol, while not occurring naturally in seed oils, can be prepared by hydrolysis, separation and esterification of the appropriate materials starting from the nut and seed oils. Fats and oils from mammalian milk are optionally metabolizable and are therefore used in with the vaccines described herein. The procedures for separation, purification, saponification and other means necessary for obtaining pure oils from animal sources are well known in the art. Fish contain metabolizable oils which are readily recovered. For example, cod liver oil, shark liver oils, and whale oil such as spermaceti can exemplify several of the fish oils which can be used herein. A number of branched chain oils can be synthesized biochemically in 5-carbon isoprene units and can be generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, can also be used. Fish oils, including squalene and squalane, can be readily available from commercial sources or can be obtained by methods known in the art.

Other useful oils include tocopherols, for use in elderly patients (e.g. aged 60 years or older) due to vitamin E been reported to have a positive effect on the immune response in this patient group. Further, tocopherols have antioxidant properties that, for example, help to stabilize the emulsions. Various tocopherols exist (α, β, γ, δ, ε or ξ) but α is usually used. An example of α-tocopherol is DL-α-tocopherol. α-tocopherol succinate can be compatible with HIV vaccines and can be a useful preservative as an alternative to mercurial compounds.

Mixtures of oils are used e.g. squalene and α-tocopherol. In some instances, an oil content in the range of 2-20% (by volume) is used.

In some instances, surfactants are classified by their ‘HLB’ (hydrophile/lipophile balance). In some cases, surfactants have a HLB of at least 10, at least 15, and/or at least 16. Surfactants can include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, such as octoxynol-9 (Triton X-100, or t-octylphenoxypolyethoxyethanol); (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl, and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants can be used herein.

Mixtures of surfactants are used e.g. Tween 80/Span 85 mixtures. A combination of a polyoxyethylene sorbitan ester and an octoxynol are also suitable. Another combination comprises, for example, laureth 9 plus a polyoxyethylene sorbitan ester and/or an octoxynol.

The amounts of surfactants (% by weight) include, for example, polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, such as 0.005 to 0.02%; polyoxyethylene ethers (such as laureth 9) 0.1 to 20%, such as 0.1 to 10% and in particular 0.1 to 1% or about 0.5%.

### Carriers and Excipients

In some instances, a vaccine further includes carriers and excipients (including, but not limited to, buffers, carbohydrates, mannitol, proteins, polypeptides or amino acids such as glycine, antioxidants, bacteriostats, chelating agents, suspending agents, thickening agents, and/or preservatives), water, oils (including those of petroleum, animal, vegetable or synthetic origin, such as peanut oil, soybean oil, mineral oil, sesame oil, and the like), saline solutions, aqueous dextrose and glycerol solutions, flavoring agents, coloring agents, detackifiers, and other acceptable additives, adjuvants, binders, or other pharmaceutically acceptable auxiliary substances as required to approximate physiological conditions (such as pH buffering agents, tonicity adjusting agents, emulsifying agents, wetting agents, and the like). Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In another instances, the pharmaceutical preparation is substantially free of preservatives. In other instances, the pharmaceutical preparation can contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)). It will be recognized that, while any suitable carrier known to those of ordinary skill in the art can be employed to administer the pharmaceutical compositions described herein, the type of carrier will vary depending on the mode of administration.

In some instances, a pharmaceutical composition of the vaccine is encapsulated within liposomes using well-known technology. Biodegradable microspheres can also be employed as carriers for the pharmaceutical compositions described herein. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

In some cases, a pharmaceutical composition is administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 2.sup.87-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

In some cases, a vaccine includes preservatives such as thiomersal or 2-phenoxyethanol. In some instances, the vaccine is substantially free from (e.g. <10 μg/ml) mercurial material e.g. thiomersal-free. In some instances, α-Tocopherol succinate is used as an alternative to mercurial compounds.

For controlling the tonicity, a physiological salt such as sodium salt are optionally included in the vaccine. Other salts include potassium chloride, potassium dihydrogen phosphate, disodium phosphate, and/or magnesium chloride, or the like.

In some instances, a vaccine has an osmolality of between 200 mOsm/kg and 400 mOsm/kg, between 240-360 mOsm/kg, or within the range of 290-310 mOsm/kg.

In some cases, a vaccine comprises one or more buffers, such as a Tris buffer; a borate buffer; a succinate buffer; a histidine buffer (particularly with an aluminum hydroxide adjuvant); or a citrate buffer. Buffers, in some cases, are included in the 5-20 mM range.

In some cases, the pH of the vaccine is between about 5.0 and about 8.5, between about 6.0 and about 8.0, between about 6.5 and about 7.5, or between about 7.0 and about 7.8.

In some instances, a vaccine is sterile. In some cases, the vaccine is non-pyrogenic e.g. containing <1 EU (endotoxin unit, a standard measure) per dose, and can be <0.1 EU per dose.

In some instances, a vaccine includes detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent can be present only at trace amounts. Thus the vaccine can include less than 1 mg/ml of each of octoxynol-10 and polysorbate 80. Other residual components in trace amounts can be antibiotics (e.g. neomycin, kanamycin, polymyxin B).

In some instances, a vaccine is formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration. Suitable formulations and additional carriers are described in Remington “The Science and Practice of Pharmacy” (20^{th} Ed., Lippincott Williams & Wilkins, Baltimore Md.), the teachings of which are incorporated by reference in their entirety herein.

In some instances, a vaccine is formulated with one or more pharmaceutically acceptable salts. Pharmaceutically acceptable salts can include those of the inorganic ions, such as, for example, sodium, potassium, calcium, magnesium ions, and the like. Such salts can include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid, or maleic acid. In addition, if the agent(s) contain a carboxy group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

Pharmaceutical compositions comprising an active agent such as small molecule fragment and/or a cysteine-containing polypeptide-small molecule fragment adduct described herein, in combination with one or more adjuvants, can be formulated to comprise certain molar ratios. For example, molar ratios of about 99:1 to about 1:99 of an active agent such as a peptide, a nucleic acid, an antibody or fragments thereof, and/or an APC described herein, in combination with one or more adjuvants, can be used. In some instances, the range of molar ratios of an active agent such as a peptide, a nucleic acid, an antibody or fragments thereof, and/or an APC described herein, in combination with one or more adjuvants, can be selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of an active agent such as a peptide, a nucleic acid, an antibody or fragments thereof, and/or an APC described herein, in combination with one or more adjuvants, can be about 1:9, and in some cases can be about 1:1. The active agent such as a peptide, a nucleic acid, an antibody or fragments thereof, and/or an APC described herein, in combination with one or more adjuvants, can be formulated together, in the same dosage unit e.g., in one vial, suppository, tablet, capsule, an aerosol spray; or each agent, form, and/or compound can be formulated in separate units, e.g., two vials, suppositories, tablets, two capsules, a tablet and a vial, an aerosol spray, and the like.

### Methods of Generating an Antibody

In some embodiments, a method of generating or raising an antibody or its binding fragment thereof comprises inoculating a mammal (e.g., a mouse, rat, or rabbit) with a small molecule fragment composition described herein. In some instances, the small molecule fragment is a small molecule fragment of Formula (I). In some instances, the method further comprises harvesting and purifying an antibody against the small molecule fragment composition.

In some embodiments, a method of generating or raising an antibody or its binding fragment thereof comprises inoculating a mammal (e.g., a mouse, rat, or rabbit) with a cysteine-containing polypeptide-small molecule fragment adduct described herein. In some instances, the cysteine-containing polypeptide-small molecule fragment adduct is a purified cysteine-containing polypeptide-small molecule fragment adduct. In some instances, the cysteine-containing polypeptide is a polypeptide illustrated in Tables 1-5. In some instances, the cysteine-containing polypeptide an isolated and purified polypeptide comprising at least 70%, 80%, 85%, 90%, 95%, 96%, 97%, 98%, 99%, or more sequence identity to at least seven contiguous amino acids of an amino acid sequence selected from SEQ ID NOs: 1-9655. In some instances, the method further comprises harvesting and purifying an antibody against the cysteine-containing polypeptide-small molecule fragment adduct.

In some instances, a method of generating or raising an antibody or its binding fragment thereof comprises inoculating a mammal (e.g., a mouse, rat, or rabbit) with a cultured cell expressing a cysteine-containing polypeptide and further administrating a small molecule fragment described herein to generate a cysteine-containing polypeptide-small molecule fragment adduct. In some instances, the cysteine-containing polypeptide is a polypeptide illustrated in Tables 1-5. In some instances, the cysteine-containing polypeptide is an isolated and purified polypeptide comprising at least 70%, 80%, 85%, 90%, 95%, 96%, 97%, 98%, 99%, or more sequence identity to at least seven contiguous amino acids of an amino acid sequence selected from SEQ ID NOs: 1-9655. In some instances, the method further comprises harvesting and purifying an antibody against the cultured cell expressing a cysteine-containing polypeptide and further incubated with a small molecule fragment described herein.

In some instances, a method of generating or raising an antibody or its binding fragment thereof comprises inoculating a mammal (e.g., a mouse, rat, or rabbit) with dendritic-cell derived exosomes. In some instances, a dendritic-cell derived exosome comprises an antigen (e.g., a cysteine-containing polypeptide-small molecule fragment adduct) which then includes activation of the antigen-specific B-cell antibody response. In some cases, the dendritic-cell derived exosome comprises a cysteine-containing polypeptide-small molecule fragment antigen. In some cases, a method of generating or raising an antibody or its binding fragment thereof comprises inoculating a mammal (e.g., a mouse, rat, or rabbit) with dendritic-cell derived exosomes comprising a cysteine-containing polypeptide-small molecule fragment antigen. In some instances, the method further comprises harvesting and purifying an antibody against the dendritic-cell derived exosomes.

### Vaccine Formulations

In some embodiments, a vaccine described herein, in combination with one or more adjuvants, is formulated in conventional manner using one or more physiologically acceptable carriers, comprising excipients, diluents, and/or auxiliaries, e.g., which facilitate processing of the active agents into preparations that can be administered. Proper formulation depends at least in part upon the route of administration chosen. The agent(s) described herein can be delivered to a patient using a number of routes or modes of administration, including oral, buccal, topical, rectal, transdermal, transmucosal, subcutaneous, intravenous, and intramuscular applications, as well as by inhalation.

In some instances, the active agents are formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible and biodegradable, such as poly(lactic-co-glycolic)acid. These materials can be made into micro or nanospheres, loaded with drug and further coated or derivatized to provide superior sustained-release performance. Vehicles suitable for periocular or intraocular injection include, for example, suspensions of therapeutic agent in injection grade water, liposomes, and vehicles suitable for lipophilic substances. Other vehicles for periocular or intraocular injection are well known in the art.

When administration is by injection, the active agent is sometimes formulated in aqueous solutions, specifically in physiologically compatible buffers such as Hanks' solution, Ringer's solution, or physiological saline buffer. The solution can contain formulatory agents such as suspending, stabilizing and/or dispersing agents. Alternatively, the active compound can be in powder form for constitution with a suitable vehicle, e.g., sterile pyrogen-free water, before use. In another embodiment, the pharmaceutical composition does not comprise an adjuvant or any other substance added to enhance the immune response stimulated by the peptide. In another embodiment, the pharmaceutical composition comprises a substance that inhibits an immune response to the peptide. Methods of formulation are known in the art, for example, as disclosed in Remington's Pharmaceutical Sciences, latest edition, Mack Publishing Co., Easton P.

For oral administration, the active agent is sometimes formulated readily by combining the active agent with pharmaceutically acceptable carriers well known in the art. Such carriers enable the agents of the disclosure to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Generally, the active agents can be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

In some instances, the vaccine is formulated into aerosol solutions, suspensions, or dry powders. The aerosol can be administered through the respiratory system or nasal passages. For example, one skilled in the art will recognize that a composition of the present disclosure can be suspended or dissolved in an appropriate carrier, e.g., a pharmaceutically acceptable propellant, and administered directly into the lungs using a nasal spray or inhalant. For example, an aerosol formulation comprising a transporter, carrier, or ion channel inhibitor can be dissolved, suspended or emulsified in a propellant or a mixture of solvent and propellant, e.g., for administration as a nasal spray or inhalant. Aerosol formulations can contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

In some instances, an aerosol formulation for inhalations and inhalants are designed so that the agent or combination of agents is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the disclosure include, for example, propane, isobutane, n-butane, pentane, isopentane, and neopentane. A blend of hydrocarbons can also be used as a propellant. Ether propellants include, for example, dimethyl ether as well as the ethers. An aerosol formulation in some instances also comprises more than one propellant. For example, the aerosol formulation can comprise more than one propellant from the same class, such as two or more fluorocarbons; or more than one, more than two, more than three propellants from different classes, such as a fluorohydrocarbon and a hydrocarbon. In some instances, vaccines are also dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

In some instances, the aerosol formulation is packaged under pressure and is formulated as an aerosol using solutions, suspensions, emulsions, powders and semisolid preparations. For example, a solution aerosol formulation can comprise a solution of an agent of the disclosure such as a transporter, carrier, or ion channel inhibitor in (substantially) pure propellant or as a mixture of propellant and solvent. The solvent can be used to dissolve the agent and/or retard the evaporation of the propellant. Solvents can include, for example, water, ethanol, and glycols. Any combination of suitable solvents can be used, optionally combined with preservatives, antioxidants, and/or other aerosol components.

In some instances, an aerosol formulation is a dispersion or suspension. A suspension aerosol formulation can comprise a suspension of an agent or combination of agents of the instant disclosure, e.g., a transporter, carrier, or ion channel inhibitor, and a dispersing agent. Dispersing agents can include, for example, sorbitan trioleate, oleyl alcohol, oleic acid, lecithin, and corn oil. A suspension aerosol formulation can also include lubricants, preservatives, antioxidant, and/or other aerosol components.

In some cases, an aerosol formulation is formulated as an emulsion. An emulsion aerosol formulation can include, for example, an alcohol such as ethanol, a surfactant, water, and a propellant, as well as an agent or combination of agents of the disclosure, e.g., a transporter, carrier, or ion channel. The surfactant used can be nonionic, anionic or cationic. One example of an emulsion aerosol formulation comprises, for example, ethanol, surfactant, water, and propellant. Another example of an emulsion aerosol formulation comprises, for example, vegetable oil, glyceryl monostearate and propane.

### Vaccine Dosages, Routes of Administration and Therapeutic Regimens

In some instances, a vaccine is delivered via a variety of routes. Exemplary delivery routes include oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous, and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999). The vaccine described herein can be administered to muscle, or can be administered via intradermal or subcutaneous injections, or transdermally, such as by iontophoresis. Epidermal administration of the vaccine can be employed.

In some instances, the vaccine is formulated for administration via the nasal passages. Formulations suitable for nasal administration, wherein the carrier is a solid, can include a coarse powder having a particle size, for example, in the range of about 10 to about 500 microns, which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

In some cases, the vaccine is a liquid preparation such as a suspension, syrup or elixir. The vaccine can also be a preparation for parenteral, subcutaneous, intradermal, intramuscular, or intravenous administration (e.g., injectable administration), such as a sterile suspension or emulsion.

In some instances, the vaccine includes material for a single immunization, or may include material for multiple immunizations (i.e. a ‘multidose’ kit). The inclusion of a preservative is preferred in multidose arrangements. As an alternative (or in addition) to including a preservative in multidose compositions, the compositions can be contained in a container having an aseptic adaptor for removal of material.

In some instances, the vaccine is administered in a dosage volume of about 0.5 mL, although a half dose (i.e. about 0.25 mL) can be administered to children. Sometimes the vaccine can be administered in a higher dose e.g. about 1 ml.

In some instances, the vaccine is administered as a 1, 2, 3, 4, 5, or more dose-course regimen. Sometimes, the vaccine is administered as a 2, 3, or 4 dose-course regimen. Sometimes the vaccine is administered as a 2 dose-course regimen.

In some instances, the administration of the first dose and second dose of the 2 dose-course regimen are separated by about 0 day, 1 day, 2 days, 5 days, 7 days, 14 days, 21 days, 30 days, 2 months, 4 months, 6 months, 9 months, 1 year, 1.5 years, 2 years, 3 years, 4 years, or more.

In some instances, the vaccine described herein is administered every 1, 2, 3, 4, 5, 6, 7, 8, 9, 10, or more years. Sometimes, the vaccine described herein is administered every 2, 3, 4, 5, 6, 7, or more years. Sometimes, the vaccine described herein is administered every 4, 5, 6, 7, or more years. Sometimes, the vaccine described herein is administered once.

The dosage examples are not limiting and are only used to exemplify particular dosing regiments for administering a vaccine described herein. The effective amount for use in humans can be determined from animal models. For example, a dose for humans can be formulated to achieve circulating, liver, topical, and/or gastrointestinal concentrations that have been found to be effective in animals. Based on animal data, and other types of similar data, those skilled in the art can determine the effective amounts of a vaccine composition appropriate for humans.

The effective amount when referring to an agent or combination of agents will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

In some instances, the vaccine is administered before, during, or after the onset of a symptom associated with a disease or condition (e.g., a cancer). Exemplary symptoms can include fever, cough, sore throat, runny and/or stuffy nose, headaches, chills, fatigue, nausea, vomiting, diarrhea, pain, or a combination thereof. In some instances, a vaccine is administered for treatment of a cancer. In some cases, a vaccine is administered for prevention, such as a prophylactic treatment of a cancer. In some cases, a vaccine is administered to illicit an immune response from a patient.

In some aspects, a vaccine and kit described herein are stored at between 2° C. and 8° C. In some instances, a vaccine is not stored frozen. In some instances, a vaccine is stored in temperatures of such as at −20° C. or −80° C. In some instances, a vaccine is stored away from sunlight.

### Pharmaceutical Compositions and Formulations

In some embodiments, disclosed herein include pharmaceutical composition and formulations comprising a small molecule fragment of Formula (I). In some instances, also described herein include pharmaceutical composition and formulations comprising a cysteine-containing polypeptide-small molecule fragment adduct. In some instances, the cysteine-containing polypeptide is a polypeptide illustrated in Tables 1-5. In other instances, the cysteine-containing polypeptide is an isolated and purified polypeptide comprising at least 70%, 80%, 85%, 90%, 95%, 96%, 97%, 98%, 99% or more sequence identity to at least seven contiguous amino acids of an amino acid sequence selected from SEQ ID NOs: 1-9655. In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronica (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and the like.

### Therapeutic Regimens for a Pharmaceutical Composition

In some embodiments, pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a small molecule fragment disclosed herein or an antibody that recognizes a cysteine-containing polypeptide-small molecule fragment adduct described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a f-pleated-sheet configuration, connected by three CDRs, which form loops connecting, and in some cases forming part of, the 3-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises, amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. It is understood that the alkyl group is acyclic. In some instances, the alkyl group is branched or unbranched. In some instances, the alkyl group is also substituted or unsubstituted. For example, the alkyl group is substituted with one or more groups including, but not limited to, alkyl, cycloalkyl, alkoxy, amino, ether, halide, hydroxy, nitro, silyl, sulfo-oxo, or thiol. A “lower alkyl” group is an alkyl group containing from one to six (e.g., from one to four) carbon atoms. In some instances, the term alkyl group is also a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

The term “aryl” as used herein is a group that contains any carbon-based aromatic group including, but not limited to, benzene, naphthalene, phenyl, biphenyl, anthracene, and the like. The aryl group can be substituted or unsubstituted. In some instances, the aryl group is substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol. The term “biaryl” is a specific type of aryl group and is included in the definition of “aryl.” In addition, the aryl group is optionally a single ring structure or comprise multiple ring structures that are either fused ring structures or attached via one or more bridging groups such as a carbon-carbon bond. For example, biaryl refers to two aryl groups that are bound together via a fused ring structure, as in naphthalene, or are attached via one or more carbon-carbon bonds, as in biphenyl.

### EXAMPLES

These examples are provided for illustrative purposes only and not to limit the scope of the claims provided herein.

### Example 1

### General Synthetic Methods

Chemicals and reagents were purchased from a variety of vendors, including Sigma Aldrich, Acros, Fisher, Fluka, Santa Cruz, CombiBlocks, BioBlocks, and Matrix Scientific, and were used without further purification, unless noted otherwise. Anhydrous solvents were obtained as commercially available pre-dried, oxygen-free formulations. Flash chromatography was carried out using 230-400 mesh silica gel. Preparative thin layer chromotography (PTLC) was carried out using glass backed PTLC plates 500-2000 μm thickness (Analtech). All reactions were monitored by thin layer chromatography carried out on 0.25 mm E. Merck silica gel plates (60F-254) and visualized with UV light, or by ninhydrin, ethanolic phosphomolybdic acid, iodine, p-anisaldehyde or potassium permanganate stain. NMR spectra were recorded on Varian INOVA-400, Bruker DRX-600 or Bruker DRX-500 spectrometers in the indicated solvent. Multiplicities are reported with the following abbreviations: s singlet; d doublet; t triplet; q quartet; p pentet; m multiplet; br broad. Chemical shifts were reported in ppm relative to TMS and J values were reported in Hz. Mass spectrometry data were collected on a HP1100 single-quadrupole instrument (ESI; low resolution) or an Agilent ESI-TOF instrument (HRMS).

In some embodiments, General Procedure A was used for the synthesis of one or more of the small molecule fragments and/or cysteine-reactive probes described herein. The amine was dissolved in anhydrous CH₂Cl₂ (0.2 M) and cooled to 0° C. To this, anhydrous pyridine (1.5 equiv.) was added in one portion, then chloroacetyl chloride (1.5 equiv.) dropwise and the reaction was monitored by TLC until complete disappearance of starting material and conversion to product was detected (typically 1 h). If the reaction did not proceed to completion, additional aliquots of pyridine (0.5 equiv.) and chloroacetyl chloride (0.5 equiv.) were added. The reaction was quenched with H₂O (1 mL), diluted with CH₂Cl₂ (20 mL), and washed twice with saturated NaHCO₃ (100 mL). The organic layer was concentrated in vacuo and purified by preparatory thin layer or flash column chromatography to afford the desired product. In some embodiments, General Procedure A1 is similar to General Procedure A except triethylamine (3 equiv.) was used instead of pyridine. In some embodiments, General Procedure A2 is similar to General Procedure A except N-methylmorpholine (3 equiv.) was used instead of pyridine.

In some embodiments, General Procedure B was used for the synthesis of one or more of the small molecule fragments and/or cysteine-reactive probes described herein. The amine was dissolved in anhydrous CH₂Cl₂ (0.2 M) and cooled to 0° C. To this, triethylamine (TEA, 1.5 equiv.), was added in one portion, then acryloyl chloride (1.5 equiv.) dropwise, and the reaction was monitored by TLC until complete disappearance of starting material and conversion to product was detected (typically 1 h). If the reaction did not proceed to completion, additional aliquots of TEA (0.5 equiv.) and acryloyl chloride (0.5 equiv.) were added. The reaction was quenched with H₂O (1 mL), diluted with CH₂Cl₂ (20 mL), and washed twice with saturated NaHCO₃ (100 mL). The organic layer was passed through a plug of silica, after which, the eluant was concentrated in vacuo and purified by preparatory thin layer or flash column chromatography to afford the desired product.

In some embodiments, General Procedure C was used for the synthesis of one or more of the small molecule fragments and/or cysteine-reactive probes described herein. Acryloyl chloride (80.4 μL, 1.0 mmol, 2 equiv.) was dissolved in anhydrous CH₂Cl₂ (4 mL) and cooled to 0° C. A solution of the amine (0.5 mmol, 1 equiv.) and N-methylmorpholine (0.16 mL, 1.5 mmol, 3 equiv.) in CH₂Cl₂ (2 mL) was then added dropwise. The reaction was stirred for 1 hr at 0° C. then allowed to warm up to room temperature slowly. After TLC analysis showed disappearance of starting material, or 6 h, whichever was sooner, the reaction was quenched with saturated aqueous NaHCO₃ (5 mL) and extracted with CH₂Cl₂ (3×10 mL). The combined organic layers were dried over anhydrous Na₂SO₄, concentrated in vacuo, and the residue obtained was purified by preparatory thin layer chromatography to afford the desired product.

### Synthesis of Probes and Fragments

### Purchased Fragments

The following electrophilic fragments were purchased from the indicated vendors. 2 (Santa Cruz Biotechnology sc-345083), 3 (Key Organics JS-092C), 4 (Sigma Aldrich T142433-10 mg), 6 (Toronto Research Chemicals M320600), 8 (Alfa Aesar H33763), 10 (Santa Cruz Biotechnology sc-345060), 11 (Santa Cruz Biotechnology sc-354895), 12 (Santa Cruz Biotechnology sc-354966), 21 (Santa Cruz Biotechnology, sc-279681), 22 (Sigma Aldrich 699357-5G), 26 (Sigma Aldrich T109959), 27 (Santa Cruz Biotechnology sc-342184), 28 (Santa Cruz Biotechnology sc-335173), 29 (Santa Cruz Biotechnology sc-348978), 30 (Santa Cruz Biotechnology sc-355362), 32 (Santa Cruz Biotechnology sc-354613), 33 (Sigma Aldrich R996505), 34 (Santa Cruz Biotechnology sc-355477), 35 (Santa Cruz Biotechnology sc-328985), 41 (Sigma Aldrich L469769), 42 (Sigma Aldrich R901946), 43 (Santa Cruz Biotechnology sc-307626), 52 (Enamine, EN300-08075), 55 (Santa Cruz Biotechnology sc-354880), 57 (VWR 100268-442), 58 (Enzo Life Sciences ALX-430-142-M005), 62 (WuXi Apptec). Synthesis of isotopically-labeled TEV-tags:

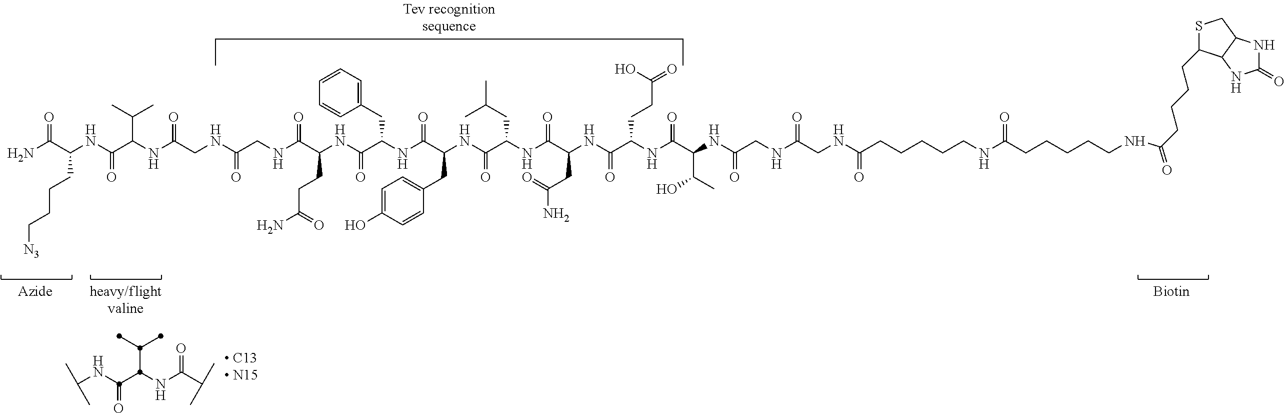

Isotopically-labeled heavy and light tags were synthesized with minor modifications to the procedure reported in Weerapana et al. Nat Protoc 2:1414-1425 (2007) and Weerapana et al. Nature 468:790-795 (2010). Fmoc-Rink-Amide-MBHA resin (EMD Biosciences; 0.5 M, 830 mg, 0.6 mmol/g loading) was deprotected with 4-methylpiperidine in DMF (50% v/v, 2×5 mL, 1 min). Fmoc-Lys(N₃)—OH (Anaspec) (500 mg, 1.26 mmol, 1.26 equiv.) was coupled to the resin overnight at room temperature with DIEA (113 μl) and 2-(6-chloro-1H-benzotriazole-1-yl)-, 1,3,3-tetramethylaminium hexafluorophosphate (HCTU; 1.3 mL of 0.5 M stock in DMF) followed by a second overnight coupling with Fmoc-Lys(N₃)—OH (500 mg, 1.26 mmol, 1.26 equiv.), DIEA (113 μl), O-(7-azabenzotriazol-1-yl)-N,N,N′,N-tetramethyluronium hexafluorophosphate (HATU; 1.3 mL of 0.5 M stock in DMF). Unmodified resin was then capped (2×30 min) with Ac₂P (400 μL) and DIEA (700 μL) in DMF after which the resin was washed with DMF (2×1 min). Deprotection with 4-methylpiperidine in DMF (50% v/v, 2×5 mL, 1 min) and coupling cycles (4 equiv. Fmoc-protected amino acid (EMD biosciences) in DMF) with HCTU (2 mL, 0.5 M in DMF) and DIEA (347.7 μL) were then repeated for the remaining amino acids. For the heavy TEV-tag, Fmoc-Valine-OH (¹³C₅C₁₅H₂₁¹⁵NO₄, ¹³C₅, 97-99%, ¹⁵N, 97-99%, Cambridge Isotope Laboratories, Inc.) was used. Reactions were monitored by ninhydrin stain and dual couplings were used for all steps that did not go to completion. Biotin (0.24 g, 2 equiv.) was coupled for two days at room temperature with NHS (0.1 g, 2 equiv.), DIC (0.16 g, 2 equiv.) and DIEA (0.175 g, 2 equiv.). The resin was then washed with DMF (5 mL, 2×1 min) followed by 1:1 CH₂Cl₂:MeOH (5 mL, 2×1 min), dried under a stream of nitrogen and transferred to a round-bottom flask. The peptides were cleaved for 90 minutes from the resin by treatment with 95:2.5:2.5 trifluoroacetic acid: water:triisopropylsilane. The resin was removed by filtration and the remaining solution was triturated with cold ether to provide either the light or heavy TEV-tag as a white solid. HPLC-MS revealed only minor impurities and the compounds were used without further purification. HRMS-ESI (m/z): calculated for C₈₃H₁₂₈N₂₃O₂₃S [M+H]: (Light-TEV-Tag) 1846.9268; found: 1846.9187; calculated for C₇₈¹³C₅H₁₂₈N₂₂¹⁵NO₂₃S [M+H]: (Heavy-TEV-Tag): 1852.9237; found: 1852.9309.

### Synthesis of Probes and Fragments

### Synthesis of 1

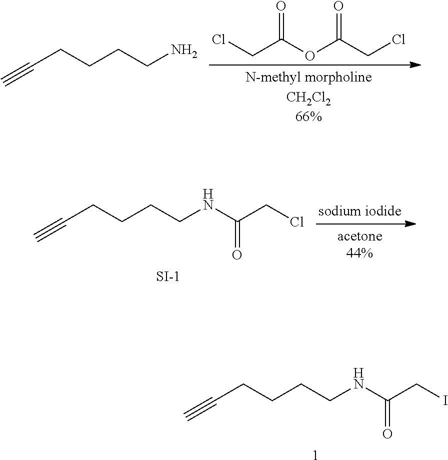

### N-(hex-5-yn-1-yl)-2-chloroacetamide (SI-1)

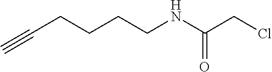

To a solution of 5-hexenylamine (63 mg, 0.65 mmol, 1.0 equiv.) in CH₂Cl₂ (3.2 mL, 0.2 M) at 0° C. was added N-methylmorpholine (215 μL, 3 equiv.) followed by chloroacetic anhydride portionwise (222 mg, 2 equiv.). The reaction was allowed to come to room temperature and then stirred overnight. The reaction was then diluted with ether (50 mL), washed with 1 M HCl, 1 M NaOH, then brine (20 mL each). The combined organic layers were dried over magnesium sulfate and concentrated to yield chloroacetamide SI-1 (74 mg, 66%). ¹H NMR (400 MHz, Chloroform-d) δ 6.79 (s, 1H), 4.09 (d, J=1.1 Hz, 2H), 3.34 (q, J=6.8 Hz, 2H), 2.23 (td, J=6.9, 2.7 Hz, 2H), 1.98 (t, J=2.7 Hz, 1H), 1.75-1.62 (m, 4H), 1.62-1.51 (m, 2H).

### N-(hex-5-yn-1-yl)-2-iodoacetamide (1)

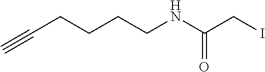

To a solution of chloroacetamide SI-1 (36.1 mg, 0.2 mmol) in acetone (1 mL, 0.2 M) was added sodium iodide (47 mg, 1.5 equiv.) and the reaction was stirred overnight. The next day the reaction was filtered through a plug of silica eluting with 20% ethyl acetate in hexanes, and the filtrate was concentrated to yield a 10:1 mixture of the desired iodoacetamide 1 and starting material. This mixture was re-subjected to the reaction conditions for one further day, at which point complete conversion was observed. The product was purified by silica gel chromatography, utilizing a gradient of 5 to 10 to 15 to 20% ethyl acetate in hexanes to yield the desired product (24 mg, 44%). In some embodiments, the reaction is performed with 2.5 equiv. of sodium iodide, in which case re-subjection is not necessary, and purification by PTLC is accomplished in 30% EtOAc/hexanes as eluent. ¹H NMR (500 MHz, Chloroform-d) δ 6.16 (s, 1H), 3.69 (s, 2H), 3.30 (q, J=6.8 Hz, 2H), 2.23 (td, J=6.8, 2.6 Hz, 2H), 1.97 (t, J=2.6 Hz, 1H), 1.75-1.61 (m, 2H), 1.61-1.52 (m, 2H). N-(4-bromophenyl)-N-phenylacrylamide (5)

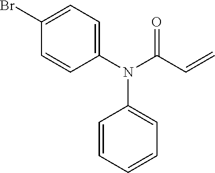 [SMILES: C=CC(=O)N(c1ccccc1)c1ccc(Br)cc1]

The title compound was synthesized according to General Procedure C from 4-bromophenylaniline (18.9 mg, 0.0762 mmol, 1 equiv.). Purification of the crude product by prep. TLC (30% EtOAc/hexanes) provided the title compound as a white solid (12.5 mg, 54%). ¹H NMR (500 MHz, Chloroform-d) δ 7.47 (d, J=8.2 Hz, 2H), 7.39 (t, J=7.6 Hz, 2H), 7.32 (d, J=7.4 Hz, 1H), 7.21 (d, J=7.7 Hz, 2H), 7.12 (d, J=8.2 Hz, 2H), 6.48 (d, J=16.7 Hz, 1H), 6.17 (dd, J=16.8, 10.3 Hz, 1H), 5.65 (d, J=10.3 Hz, 1H); HRMS-ESI (m/z) calculated for C₁₅H₁₃BrNO [M+H]: 302.0175; found: 302.0176.

### Synthesis of 7

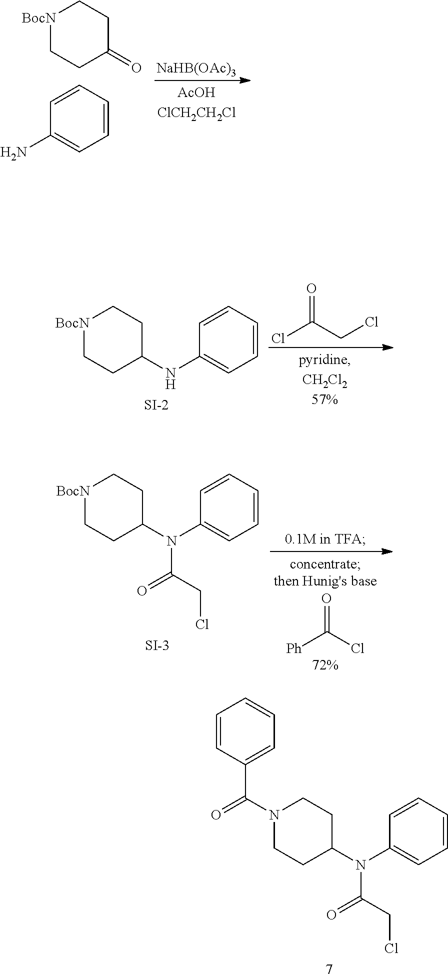

### tert-butyl 4-(phenylamino)piperidine-1-carboxylate (SI-2)

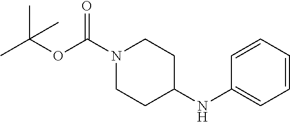 [SMILES: CC(C)(C)OC(=O)N1CCC(Nc2ccccc2)CC1]

SI-2 was prepared according to Thoma et al, J. Med. Chem. 47:1939-1955 (2004). ¹H NMR (400 MHz, Chloroform-d) δ 7.24-7.12 (m, 2H), 6.75-6.68 (m, 1H), 6.66-6.58 (m, 2H), 3.88-3.81 (m, 1H), 3.44 (tt, J=10.4, 3.9 Hz, 2H), 3.00-2.88 (m, 2H), 2.10-1.99 (m, 2H), 1.48 (bs 9H), 1.41-1.27 (m, 2H).

### tert-butyl 4-(2-chloro-N-phenylacetamido)piperidine-1-carboxylate (SI-3)

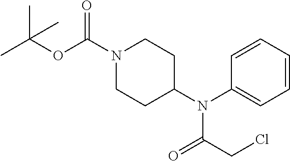 [SMILES: CC(C)(C)OC(=O)N1CCC(N(C(=O)CCl)c2ccccc2)CC1]

To a solution of aniline SI-2 (65 mg, 0.24 mmol) at 0° C. in CH₂Cl₂ (0.6 mL) was added pyridine (38 μL, 2 equiv.) followed by chloroacetyl chloride (37.4 μL, 2.0 equiv.) in CH₂Cl₂ (0.6 mL). The resulting solution was allowed to warm to room temperature and stirred overnight. The solution was then quenched with saturated aqueous sodium bicarbonate, extracted with Et₂O (3×10 mL). The combined organic layers were dried over magnesium sulfate, filtered and concentrated to give an off-white solid, which was used without further purification (47 mg, 57%). ¹H NMR (400 MHz, Chloroform-d) δ 7.47-7.38 (m, 3H), 7.18-7.03 (m, 2H), 4.75-4.63 (m, 1H), 4.07 (s, 2H), 3.68 (s, 2H), 2.76 (s, 2H), 1.84-1.69 (m, 2H), 1.35 (s, 9H), 1.27-1.12 (m, 2H).

### N-(1-benzoylpiperidin-4-yl)-2-chloro-N-phenylacetamide (7)

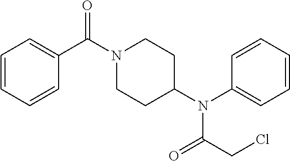 [SMILES: O=C(c1ccccc1)N1CCC(N(C(=O)CCl)c2ccccc2)CC1]

To neat SI-3 (47 mg, 0.128 mmol) was added trifluoroacetic acid (0.7 mL, final 0.2 M). The resulting solution was concentrated under a stream of nitrogen until no further evaporation was observed, providing the deprotected amine as its trifluoroacetate salt. This viscous gum was then treated with triethylamine in ethyl acetate (10% v/v, 2 mL; solution smokes upon addition). The resulting solution was concentrated to afford the free base, which contained only triethylammonium trifluoroacetate and the free amine by proton NMR. A stock solution was prepared by dissolving the resulting gum in CH₂Cl₂ (1.2 mL, ˜0.1 M final).

The deprotected amine (0.3 mL of stock solution, 0.0319 mmol) was treated with Hunig's base (17.5 μL, 3 equiv.) and benzoyl chloride (7.6 μL, 2.0 equiv.). This solution was stirred overnight, quenched with saturated aqueous sodium bicarbonate, extracted with Et₂O (3×10 mL). The resulting solution was dried over magnesium sulfate, filtered and concentrated. The resulting oil was purified by silica gel chromatography (20% EtOAc/hexanes) to afford chloroacetamide 7 as a white solid (8.6 mg, 75%). ¹H NMR (500 MHz, Chloroform-d) δ 7.55 (dd, J=5.5, 3.0 Hz, 3H), 7.50-7.32 (m, 5H), 7.21 (s, 2H), 4.92 (tt, J=12.3, 4.0 Hz, 1H), 4.87 (s, 1H), 3.87 (s, 1H), 3.78 (s, 2H), 3.21 (s, 1H), 2.97-2.90 (m, 1H), 2.01 (s, 1H), 1.90 (s, 1H), 1.45 (s, 1H), 1.36-1.26 (m, 1H); HRMS-ESI (m/z) calculated for C₂₀H₂₂ClN₂O₂ [M+H]: 357.1364; found: 357.1362.

### 1-(4-benzylpiperidin-J-yl)-2-chloroethan-1-one (9)

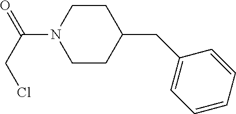 [SMILES: O=C(CCl)N1CCC(Cc2ccccc2)CC1]

Following General Procedure A, starting from 4-benzylpiperidine (840 mg, 5.2 mmol, 1 equiv.), the desired compound was obtained after column chromatography as a yellow oil (1 g, 81%). Spectroscopic data matches those reported previously reported in Papadopoulou et al. J. Med. Chem. 55:5554-5565 (2012). ¹H NMR (500 MHz, Chloroform-d) δ 7.42-7.14 (m, 5H), 4.61 (d, J=13.4 Hz, 1H), 4.14 (q, J=21.9, 11.5 Hz, 2H), 3.89 (d, J=13.5, 1H), 3.11 (td, J=13.1, 2.7 Hz, 1H), 2.69-2.57 (m, 3H), 1.92-1.75 (m, 3H), 1.40-1.21 (m, 2H); HRMS-ESI (m/z) calculated for C₁₄H₁₉ClNO [M+H]: 252.115; found: 252.115.

### N-(2-(1H-indol-3-yl)ethyl)-2-chloroacetamide (13)

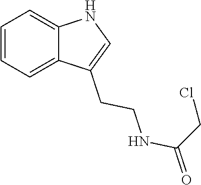 [SMILES: O=C(CCl)NCCc1c[nH]c2ccccc12]

Following General Procedure A, starting from tryptamine (400 mg, 2.5 mmol, 1 equiv.), the desired compound was obtained after column chromatography as a brownish solid (460 mg, 77%). ¹H NMR (500 MHz, Chloroform-d) δ 8.55 (s, 1H), 7.70 (d, J=7.9 Hz, 1H), 7.45 (d, J=8.1 Hz, 1H), 7.30 (t, J=7.5 Hz, 1H), 7.23 (t, J=7.4 Hz, 1H), 7.10 (s, 1H), 6.84 (s, 1H), 4.08 (s, 2H), 3.72 (q, J=6.4 Hz, 2H), 3.10 (t, J=6.8 Hz, 2H); HRMS-ESI (m/z) calculated for C₁₂H₁₄ClN₂O₂ [M+H]: 237.0789; found: 237.0791.

### N-(3,5-bis(trifluoromethyl)phenyl)acrylamide (14)

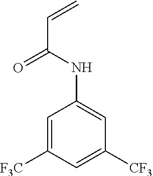 [SMILES: C=CC(=O)Nc1cc(C(F)(F)F)cc(C(F)(F)F)c1]

Following General Procedure B, starting from 3,5-bis(trifluoromethyl)aniline (1.16 g, 5 mmol, 1 equiv.), the desired compound was obtained after column chromatography as a white solid (1.05 g, 74%). ¹H NMR (500 MHz, Chloroform-d) δ 8.33 (s, 1H), 8.18 (s, 2H), 7.68 (s, 1H), 6.57 (d, J=17.5 Hz, 1H), 6.38 (dd, J=16.9, 10.3 Hz, 1H), 5.93 (d, J=12.5 Hz, 1H); HRMS-ESI (m/z) calculated for C₁₁H₈F₆NO₂ [M+H]: 284.0505; found: 284.0504.

### N-(4-phenoxy-3-(trifluoromethyl)phenyl)-N-(pyridin-3-ylmethyl)acrylamide (15)

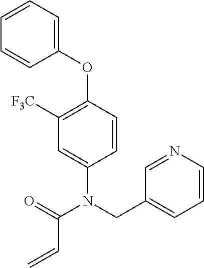 [SMILES: C=CC(=O)N(Cc1cccnc1)c1ccc(Oc2ccccc2)c(C(F)(F)F)c1]

4-phenoxy-3-(trifluoromethyl)aniline (260 mg, 1 mmol, 1 equiv.) (Combi-Blocks) was dissolved in TFA (5 mL). Following the reductive amination protocol reported by Boros et al. J. Org. Chem 74:3587-3590 (2009), the reaction mixture was cooled to 0° C. and to this sodium triacetoxyborohydride (STAB) (270 mg, 1.3 mmol, 1.3 equiv.) was added. 3-pyridinecarboxaldehyde (200 mg, 2 mmol, 2 equiv.) was dissolved in CH₂Cl₂ (5 mL) and slowly added to the reaction mixture. Upon complete conversion to product, the reaction was diluted with CH₂Cl₂ (20 mL) and washed with saturated sodium bicarbonate solution (3×20 mL) and the organic layer was dried then concentrated under reduced pressure. Without further purification the crude material was dissolved in anhydrous CH₂Cl₂ and subjected to General Procedure B. The resulting crude was purified by prep. TLC to give a white solid (31 mg, 10%). ¹H NMR (500 MHz, Chloroform-d) δ 8.52 (d, J=3.5 Hz, 1H), 8.39 (s, 1H), 7.68 (d, J=7.8. Hz, 1H), 7.40 (t, J=7.7 Hz, 2H), 7.34 (s, 1H), 7.28-7.18 (m, 2H), 7.07 (d, J=8.2 Hz, 2H), 6.98 (d, J=7.5 Hz, 1H), 6.82 (d, J=8.8 Hz, 1H), 6.46 (d, J=16.8 Hz, 1H), 6.01 (dd, J=16.2, 10.7 Hz, 1H), 5.64 (d, J=10.3 Hz, 1H), 4.96 (s, 2H). HRMS-ESI (m/z) calculated for C₂₂H₁₈F₃N₂O₂ [M+H]: 399.1315; found: 399.1315.

### Iodoacetamide-rhodamine (16)

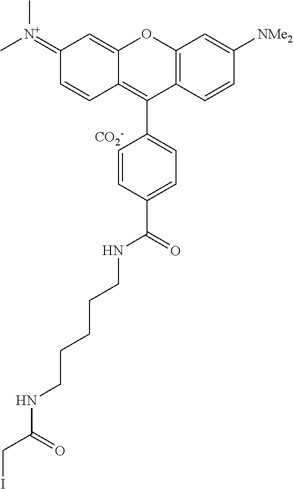

5-(and-6)-((N-(5-aminopentyl)amino)carbonyl)tetramethylrhodamine (tetramethylrhodamine cadaverine) mixed isomers (60 mg, 0.12 mmol, 1 equiv.) were dissolved in anhydrous DMF (500 μL) with sonication. To this was added DIPEA (60 μL, 0.34 mmol, 3 equiv.) and chloroacetyl chloride (10 μL, 0.13 mmol, 1 equiv., diluted 1:10 in DMF) and the reaction was stirred at room temperature for 20 min until complete conversion to the product was detected by TLC. The DMF was removed under a stream of nitrogen and the reaction mixture was separated by PTLC in MeOH:CH₂Cl₂:TEA (15:85:0.001). The chloroacetamide rhodamine was then eluted in MeOH:CH₂Cl₂ (15:85), concentrated under reduced pressure and redissolved in acetone (500 μL). NaI (150 mg, 1 mmol, 10 equiv.) was added to this and the reaction was stirred for 20 min at 50° C. until complete conversion to product was detected and the crude reaction mixture was purified by reverse phase HPLC on a C18 column and concentrated to yield the title compound as a purple solid that is a mixture of 5 and 6 carboxamide tetramethylrhodamine isomers (ratio˜6:1) (10 mg, 12%). ¹H NMR (600 MHz, Methanol-d₄) δ 8.87 (t, J=4.8 Hz, 0.14H), 8.80-8.71 (m, 1H), 8.41 (dd, J=8.2, 1.1 Hz, 0.86H), 8.35 (br s, 1H), 8.27 (dt, J=7.9, 1.5 Hz, 0.164H), 8.20 (dt, J=8.2, 1.5 Hz, 0.86H), 7.81 (s, 0.86H), 7.53 (d, J=7.8 Hz, 0.14H), 7.18-7.11 (m, 2H), 7.07 (d, J=9.5 Hz, 2H), 7.00 (s, 2H), 3.68-3.62 (m, 2H), 3.46-3.37 (m, 2H), 3.31 (s, 12H, obscured by solvent) 3.21-3.12 (m, 2H), 1.81-1.21 (m, 6H); HRMS-ESI (m/z) calculated for C₃₂H₃₆N40₅ [M+H]: 683.1725; found: 683.1716.

### N-(3,5-bis(trifluoromethyl)phenyl)acetamide (17)

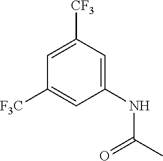 [SMILES: CC(=O)Nc1cc(C(F)(F)F)cc(C(F)(F)F)c1]

Following General Procedure A, starting with 3,5-bis(trifluoromethyl)aniline (327 mg, 1.42 mmol, 1 equiv.) and acetic anhydride (200 μL, 3 mmol, 2 equiv.), the title compound was obtained after PTLC as a white solid (302 mg, 78%). ¹H NMR (500 MHz, Chloroform-d) δ 8.10 (s, 2H), 7.72 (s, 1H), 7.68 (s, 1H), 2.32 (d, J=0.9 Hz, 3H). HRMS-ESI (m/z) calculated for C₁₁H₈F₆NO₂ [M+H]: 284.0505; found: 284.0504.

### Synthesis of 18 and 19

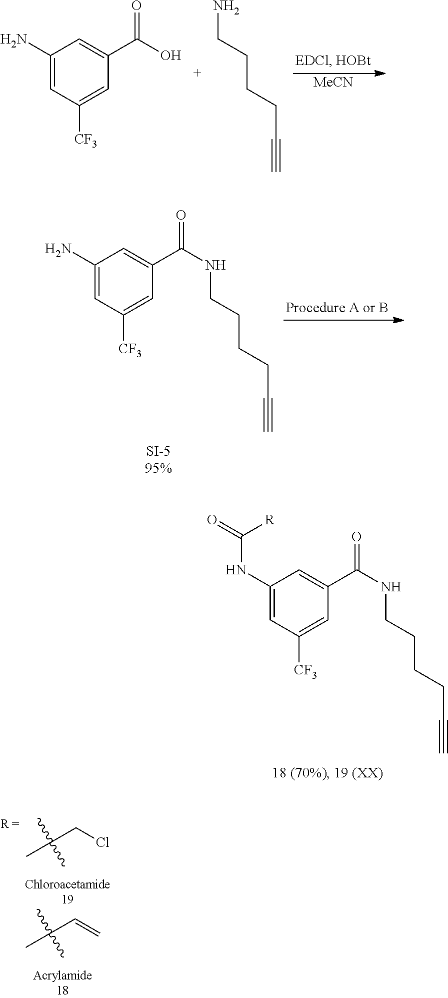

### 3-amino-N-(hex-5-yn-1-yl)-5-(trifluoromethyl)benzamide (SI-5)

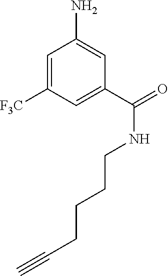 [SMILES: C#CCCCCNC(=O)c1cc(N)cc(C(F)(F)F)c1]

To a solution of 3-amino-5-(trifluoromethyl)benzoic acid (74 mg, 0.36 mmol) in acetonitrile (3.6 mL, 0.1 M final) was added EDCI (83 mg, 1.2 equiv.) followed by hex-5-ynamine (35 mg, 1.0 equiv.) followed by 1-hydroxybenzotriazole hydrate (HOBt, 66.3 mg, 1.2 equiv.) and the resulting solution was stirred overnight. The reaction was diluted with ethyl acetate, washed with 1 M HCl twice and then brine. The organic layer was dried over magnesium sulfate and concentrated to yield aniline SI-5 (97.4 mg, 95%) as a white solid. ¹H NMR (400 MHz, Chloroform-d) δ 7.29-7.22 (m, 2H), 6.98 (t, J=1.8 Hz, 1H), 6.38 (t, J=5.5 Hz, 1H), 4.08 (s, 2H), 3.46 (td, J=7.1, 5.7 Hz, 2H), 2.25 (td, J=6.9, 2.6 Hz, 2H), 1.99 (t, J=2.7 Hz, 1H), 1.81-1.55 (m, 4H).

### 3-acrylamido-N-(hex-5-yn-1-yl)-5-(trifluoromethyl)benzamide (18)

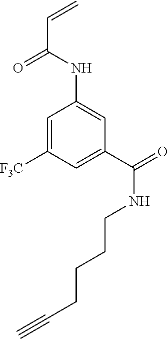 [SMILES: C#CCCCCNC(=O)c1cc(NC(=O)C=C)cc(C(F)(F)F)c1]

Following General Procedure B, starting with SI-5 (42 mg, 0.15 mmol, 1 equiv.), the title compound was obtained after column chromatography as a white solid (34 mg, 70%). ¹H NMR (500 MHz, Chloroform-d) δ 8.94 (s, 1H), 8.24 (d, J=11.9 Hz, 2H), 7.71 (s, 1H), 6.87 (t, J=5.7 Hz, 1H), 6.55 (dd, J=17.4, 0.7 Hz, 1H), 6.43 (dd, J=16.9, 10.1 Hz, 1H), 5.88 (dd, J=10.1, 1.3 Hz, 1H), 3.56 (q, J=6.7 Hz, 2H), 2.33 (td, J=6.9, 2.7 Hz, 2H), 2.06 (t, J=2.7 Hz, 1H), 1.87 (p, J=7.3 Hz, 2H), 1.69 (p, J=7.8 Hz, 2H); HRMS-ESI (m/z) calculated for C₁₇H₁₈F₃N₂O₂ [M+H]: 339.1314; found 339.1313.

### 3-acrylamido-N-(hex-5-yn-1-y)-5-(trifluoromethyl)benzamide (19)

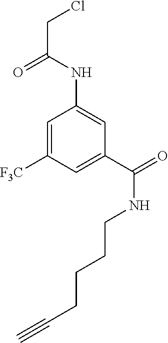 [SMILES: C#CCCCCNC(=O)c1cc(NC(=O)CCl)cc(C(F)(F)F)c1]

Synthesized according to General Procedure A2, starting from SI-5. ¹H NMR (600 MHz, Chloroform-d) δ 8.57 (s, 1H), 8.16 (t, J=1.8 Hz, 1H), 8.05 (t, J=1.8 Hz, 1H), 7.79 (d, J=2.0 Hz, 1H), 6.38 (d, J=6.1 Hz, 1H), 4.23 (s, 2H), 3.51 (td, J=7.1, 5.7 Hz, 2H), 2.27 (td, J=6.9, 2.7 Hz, 2H), 2.00 (t, J=2.6 Hz, 1H), 1.82-1.74 (m, 2H), 1.71-1.59 (m, 2H); HRMS-ESI (m/z) calculated for C₁₆H₁₇ClF₃N₂O₂ [M+H]: 361.0925; found: 361.0925.

### 2-chloro-1-(4-(hydroxydiphenylmethyl)piperidin-1-yl)ethan-1-one (20)

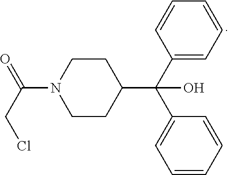 [SMILES: O=C(CCl)N1CCC(C(O)(c2ccccc2)c2ccccc2)CC1]

Following General Procedure A, starting with α,α-diphenyl-4-piperidinemethanol (800 mg, 3 mmol, 1 equiv.), the title compound was obtained after column chromatography as a white solid (637 mg, 61%). ¹H NMR (500 MHz, Chloroform-d) δ 7.56 (d, J=7.6 Hz, 4H), 7.39 (q, J=7.1 Hz, 4H), 7.28 (q, J=6.8 Hz, 2H), 4.66 (d, J=13.3 Hz, 1H), 4.07 (dd, J=12.2, 4.2 Hz, 2H), 3.91 (d, J=13.4 Hz, 1H), 3.18 (t, J=12.9 Hz, 1H), 2.77-2.62 (m, 3H), 1.67 (t, J=12.5 Hz, 2H), 1.56 (q, J=11.8 Hz, 1H), 1.44 (q, J=12.4, 11.8 Hz, 1H); HRMS-ESI (m/z) calculated for C₂₀H₂₃ClNO₂ [M+H]: 344.1412; found: 344.1412.

### (E)-3-(3,5-bis(trifluoromethyl)phenyl)-2-cyanoacrylamide (23)

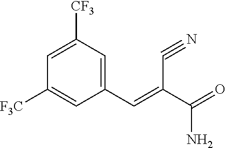 [SMILES: N#C/C(=C\c1cc(C(F)(F)F)cc(C(F)(F)F)c1)C(N)=O]

3,5-bis(trifluoromethyl)benzaldehyde (880 mg, 3.6 mmol, 1 equiv.) and 2-cyanoacetamide (460 mg, 5.5 mmol, 1.5 equiv.) were dissolved in MeOH (10 mL). To this was added piperidine (214 mg, 0.7 equiv.) and the reaction was stirred at room temperature for 30 minutes at which point starting material was consumed. After addition of an equivalent volume of water (10 mL), the precipitate was collected by filtration and washed with water/methanol (1:1) to yield the title compound as a white solid (534 mg, 47%). ¹H NMR (400 MHz, Acetone-d₆) δ 8.78 (s, 2H), 8.61 (s, 1H), 8.41 (s, 1H), 7.57 (s, 1H), 7.42 (s, 1H); HRMS-ESI (m/z) calculated for C₁₂H₇F6N₂O₂ [M+H]: 309.0457; found: 309.0459.

### N-(3,5-bis(trifluoromethyl)phenyl)-2-bromopropanamide (24)

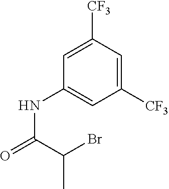 [SMILES: CC(Br)C(=O)Nc1cc(C(F)(F)F)cc(C(F)(F)F)c1]

Following General Procedure A1, starting with 3,5-bis(trifluoromethyl)aniline (250 mg, 1.1 mmol, 1 equiv.) and 2-bromopropionyl chloride (200 j±L, 2 mmol, 1.8 equiv.) the title compound was obtained by PTLC as a white solid (130 mg, 35%). ¹H NMR (500 MHz, Chloroform-d) δ 8.34 (s, 1H), 8.06 (s, 2H), 7.66 (s, 1H), 4.58 (q, J=7.0 Hz, 1H), 1.98 (d, J=7.0 Hz, 3H); HRMS-ESI (m/z) calculated for C₁₁H₇BrF₆NO [M−H]: 361.9621; found: 361.9623.

### N-(3,5-bis(trifluoromethyl)phenyl)-2-chloropropanamide (25)

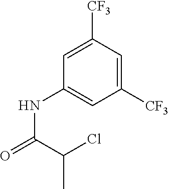 [SMILES: CC(Cl)C(=O)Nc1cc(C(F)(F)F)cc(C(F)(F)F)c1]

Following General Procedure A1, starting with 3,5-bis(trifluoromethyl)aniline (327 mg, 1.42 mmol, 1 equiv.) and 2-chloropropionyl chloride (200 μL, 2 mmol, 1.8 equiv.) the title compound was obtained by PTLC as a white solid (250 mg, 55%). ¹H NMR (500 MHz, Chloroform-d) δ 8.61 (s, 1H), 8.16 (s, 2H), 7.75 (s, 1H), 4.67 (q, J=7.1 Hz, 1H), 1.93 (d, J=7.1 Hz, 3H). HRMS-ESI (m/z) calculated for C₁₁H₇ClF₆NO [M−H]: 318.0126; found: 318.0126.

### N-(3,5-bis(trifluoromethyl)phenyl)-N-(pyridin-3-ylmethyl)acrylamide (31)

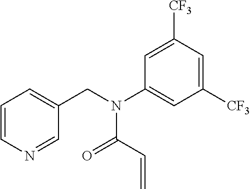 [SMILES: C=CC(=O)N(Cc1cccnc1)c1cc(C(F)(F)F)cc(C(F)(F)F)c1]

3,5-bis(trifluoromethyl)aniline (350 mg, 1.6 mmol, 1 equiv.) was dissolved in TFA (5 mL). The reaction mixture was cooled to 0° C. and to this sodium triacetoxyborohydride (STAB) (400 mg, 2 mmol, 1.3 equiv.) was added. 3-pyridinecarboxaldehyde (244 mg, 1.5 mmol, 1 equiv.) was dissolved in CH₂Cl₂ (5 mL) and slowly added to the reaction mixture dropwise over 10 minutes. Upon complete conversion to product, the reaction mixture was diluted with CH₂Cl₂ (20 mL) and washed with saturated sodium bicarbonate solution (3×20 mL) and the organic layer was dried then concentrated under reduced pressure. Without further purification the crude material was dissolved in anhydrous CH₂Cl₂ and subjected to General Procedure B. The resulting crude was purified by PTLC to give a white solid (10 mg, 2%). ¹H NMR (500 MHz, Chloroform-d) δ 8.63 (d, J=3.8 Hz, 1H), 8.49 (s, 1H), 7.93 (s, 1H), 7.70 (d, J=7.7 Hz, 1H), 7.55 (s, 2H), 7.35 (dd, J=7.6, 5.3 Hz, 1H), 6.60 (dd, J=16.6, 1.6 Hz, 1H), 6.02 (dd, J=16.9, 10.2 Hz, 1H), 5.79 (dd, J=10.3, 1.6 Hz, 1H), 5.11 (s, 2H). HRMS-ESI (m/z) calculated for C₁₇H₁₃F₆N₂O [M+H]: 375.0927; found: 375.0928.

### 3-(2-chloroacetamido)-5-(trifluoromethyl)benzoic acid (36)

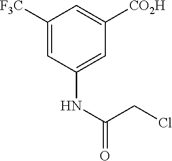 [SMILES: O=C(CCl)Nc1cc(C(=O)O)cc(C(F)(F)F)c1]

To a solution of 3-amino-5-(trifluoromethyl)benzoic acid (500 mg, 2.44 mmol) in 1.5 mL of dimethylacetamide (1.6 M) at 0° C. was added chloroacetyl chloride (214 μL, 2.69 mmol, 1.1 equiv.). The resulting solution was warmed to ambient temperature and stirred for 20 minutes, at which point ethyl acetate (40 mL) and water (30 mL) were added. The pH of the aqueous layer was adjusted to pH 10 via addition of 1 N NaOH, and the phases were separated. The aqueous layer was washed with 40 mL of ethyl acetate, then acidified by adding 1 N HCl. The product was extracted with ethyl acetate (40 mL), and the organic layer was washed with 1M HCl (2×40 mL), brine (40 mL), dried over magnesium sulfate and concentrated to provide the desired product (456 mg, 66%). ¹H NMR (500 MHz, Chloroform-d) δ 8.31 (s, 1H), 8.27 (s, 1H), 8.14 (s, 1H), 4.13 (s, 2H); HRMS-ESI (m/z) calculated for C₁₀H₈ClF₃NO₃ [M+H]: 282.0139; found: 282.0141.

### 1-(4-(5-fluorobenzisoxazol-3-yl)piperidin-1-yl)prop-2-en-1-one (37)

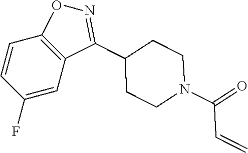 [SMILES: C=CC(=O)N1CCC(c2noc3ccc(F)cc23)CC1]

The title compound was obtained starting from 6-fluoro-3(4-piperidinyl)-1,2-benzisoxazole hydrochloride (53 mg, 0.2 mmol, 1 equiv.) according to General Procedure C as a colorless oil (49.1 mg, 87%). ¹H NMR (400 MHz, Chloroform-d) δ 7.64 (dd, J=8.7, 5.1 Hz, 1H), 7.27 (dd, J=8.4, 2.3 Hz, 1H), 7.08 (td, J=8.9, 2.1 Hz, 1H), 6.64 (dd, J=16.8, 10.6 Hz, 1H), 6.32 (dd, J=16.9, 1.9 Hz, 11H), 5.73 (dd, J=10.6, 1.9 Hz, 1H), 4.70 (d, J=13.4 Hz, 1H), 4.15 (d, J=12.4 Hz, 1H), 3.53-3.13 (m, 2H), 2.99 (t, J=13.1 Hz, 1H), 2.25-2.07 (m, 2H), 2.00 (ddd, J=23.1, 14.2, 7.8 Hz, 211); HRMS-ESI (m/z) calculated for C₁₅H₁₆FN₂O [M+H]: 275.119; found: 275.119.

### tert-butyl 4-(4-acrylamido-2,6-difluorophenyl)piperazine-1-carboxylate (38)

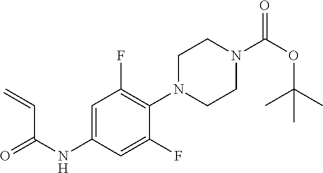 [SMILES: C=CC(=O)Nc1cc(F)c(N2CCN(C(=O)OC(C)(C)C)CC2)c(F)c1]

The title compound was obtained starting from tert-Butyl 4-(4-amino-2,6-difluorophenyl)piperazine-1-carboxylate according to General Procedure B. ¹H NMR (400 MHz, Chloroform-d) δ 8.12 (s, 1H), 7.13 (d, J=10.4 Hz, 2H), 6.36 (d, J=16.9 Hz, 1H), 6.19 (dd, J=16.8, 10.2 Hz, 1H), 5.70 (d, J=10.2 Hz, 1H), 3.45 (t, J=4.7 Hz, 4H), 3.00 (t, J=3.7 Hz, 4H), 1.41 (s, 9H); HRMS-ESI (m/z) calculated for C₁₈H₂₄F₂N₃O₃ [M+H]: 368.178; found: 368.178.

### N-(4-bromo-2,5-dimethylphenyl)acrylamide (40)

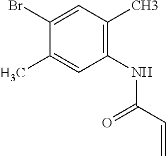

Following General Procedure B, starting from 4-bromo-2,5-dimethylaniline (900 mg, 4.5 mmol, 1 equiv.), the title compound was obtained after column chromatography and recrystallization from cold CH₂Cl₂ as a white solid (611 mg, 40%). ¹H NMR (500 MHz, Chloroform-d) δ 7.87 (s, 1H), 7.43 (s, 1H), 7.16 (s, 1H), 6.50 (d, J=16.7 Hz, 1H), 6.35 (dd, J=16.4, 10.3 Hz, 1H), 5.86 (d, J=10.3 Hz, 1H), 2.42 (s, 3H), 2.28 (s, 3H); HRMS-ESI (m/z) calculated for C₁₁H₁₃BrNO [M+H]: 254.0175; found: 254.0175.

### 2-Chloroacetamido-2-deoxy-α/β-D-glucopyranose (44)

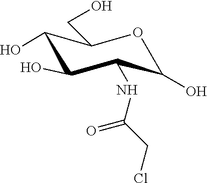

To a stirred solution of hexosamine hydrochloride (590 mg, 3.39 mmol, 1 equiv.) in anhydrous MeOH (200 mL) at room temperature was added sodium metal (60 mg, 2.6 mmol, 0.78 equiv.), TEA (400 μL, 5.7 mmol, 1.8 equiv.). Chloroacetic anhydride (1 g, 5.9 mmol, 1 equiv.) was then added and the mixture stirred for 6 h, monitoring for completeness by TLC. After which, the reaction mixture was concentrated in vacuo. The crude product then was purified by two rounds of column chromatography to afford the pure title product as a white solid (610 mg, 72%). ¹H NMR (500 MHz, Methanol-d₄) δ 5.20 (d, J=3.7 Hz, 1Hα), 4.75 (d, J=8.3 Hz, 1H3), 4.19 (dd, J=20.2, 13.9 Hz, 2H), 4.19 (d, J=12.6 Hz, 1H), 3.95 (dd, J=10.6, 3.5 Hz, 1Hα), 3.83 (m, 3Hα, 3HP), 3.74 (d, J=5.1 Hz, 1Hβ), 3.70 (dd, J=11.4, 8.9 Hz, 1Hβ), 3.60 (dd, J=10.7, 9.5 Hz, 1Hβ), 3.46 (t, J=9.3 Hz, 1H), 3.42 (t, J=10.0 Hz, 1H); HRMS-ESI (m/z) calculated for C₈H₁₅ClNO₆ [M+H]: 256.0582; found: 256.0582.

### 2-chloro-1-(2-methyl-3,4-dihydroquinolin-1 (2H)-yl)ethan-1-one (45)

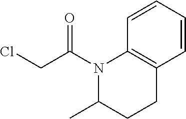

Chloroacetyl chloride (80.4 μL, 0.9 mmol, 1.7 equiv.) was dissolved in anhydrous CH₂Cl₂ (3 mL) and cooled to 0° C. A solution of 2-methyl-1,2,3,4-tetrahydroquinoline (80.1 mg, 0.544 mmol, 1 equiv.) and N-methylmorpholine (0.11 mL, 1.0 mmol, 1.8 equiv.) in CH₂Cl₂ (2 mL) was then added dropwise. After 6 h, the reaction was quenched with saturated aqueous NaHCO₃ (5 mL) and extracted with CH₂Cl₂ (3×10 mL). The combined organic layers were dried over anhydrous Na₂SO₄ and concentrated under reduced pressure. The resultant residue was purified by prep. TLC (30% EtOAc/hexanes), providing the title compound as an off-white solid (108.8 mg, 89%). ¹H NMR (400 MHz, chloroform-d) δ 7.30-7.13 (m, 4H), 4.86-4.75 (m, 1H), 4.20 (d, J=12.5 Hz, 1H), 4.09 (d, J=12.5 Hz, 1H), 2.69-2.58 (m, 1H), 2.59-2.46 (m, 1H), 2.46-2.31 (m, 1H), 1.36-1.29 (m, 1H), 1.15 (d, J=6.5 Hz, 3H); HRMS-ESI (m/z) calculated for C₂H₁₅ClNO [M+H]: 224.0837; found: 224.0836.

### N-cyclohexyl-N-phenylacrylamide (46)

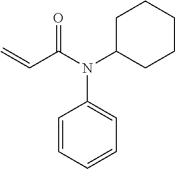 [SMILES: C=CC(=O)N(c1ccccc1)C1CCCCC1]

The title compound was synthesized according to General Procedure C from N-cyclohexylaniline (89.5 mg, 0.511 mmol, 1 equiv.). Purification of the crude product by flash column chromatography (10-20% EtOAc/hexanes) then prep. TLC (30% EtOAc/hexanes) provided the title compound as an off-white solid (53.1 mg, 45%). ¹H NMR (400 MHz, chloroform-d) δ 7.42-7.33 (m, 3H), 7.10-7.06 (m, 2H), 6.31 (dd, J=16.7, 2.1 Hz, 1H), 5.77 (dd, J=16.7, 10.3 Hz, 1H), 5.41 (dd, J=10.4, 2.1 Hz, 1H), 4.65 (tt, J=12.2, 3.7 Hz, 1H), 1.85 (dt, J=11.2, 1.8 Hz, 2H), 1.75-1.68 (m, 2H), 1.61-1.53 (m, 1H), 1.40 (qt, J=13.3, 3.6 Hz, 2H), 1.07 (qd, J=12.4, 3.6 Hz, 2H), 0.91 (qt, J=13.1, 3.8 Hz, 1H); HRMS-ESI (m/z) calculated for C₁₅H₂₀NO [M+H]: 230.1539; found: 230.1539.

### 1-(5-bromoindolin-1-yl)prop-2-en-1-one (47)

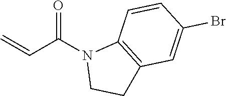

The title compound was synthesized according to General Procedure C from 5-bromoindoline (41.7 mg, 0.211 mmol, 1 equiv.), acryloyl chloride (32 μL, 0.40 mmol, 1.9 equiv.), and changing the base to pyridine (32 μL, 0.40 mmol, 1.9 equiv.). Purification of the crude product by re-precipitation from EtOAc provided the title compound as a white solid (67.8 mg, 64%). ¹H NMR (400 MHz, chloroform-d) δ 8.16 (d, J=8.6 Hz, 1H), 7.33-7.25 (m, 2H), 6.60-6.42 (m, 211), 5.84-5.76 (m, 1H), 4.15 (t, J=8.6 Hz, 2H), 3.17 (t, J=8.6 Hz, 2H); HRMS-ESI (m/z) calculated for C₁₁H₁₁BrNO [M+H]: 252.0018; found: 252.0017.

### N-(1-benzylpiperidin-4-yl)-N-phenylacrylamide (48)

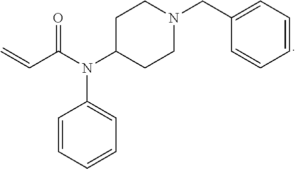 [SMILES: C=CC(=O)N(c1ccccc1)C1CCN(Cc2ccccc2)CC1]

The title compound was synthesized according to General Procedure C from 1-benzyl-N-phenylpiperidin-4-amine (30.0 mg, 0.113 mmol, 1 equiv.), acryloyl chloride (17 μL, 0.21 mmol, 1.9 equiv.), and changing the base to pyridine (17 μL, 0.21 mmol, 1.9 equiv.). Purification of the crude product by prep. TLC provided the title compound as a white solid (22.5 mg, 64%). ¹H NMR (400 MHz, chloroform-d) δ 7.62-7.56 (m, 2H), 7.43-7.36 (m, 6H), 7.05 (d, J=6.2 Hz, 2H), 6.29 (dd, J=16.8, 2.1 Hz, 1H), 5.79 (dd, J=16.8, 10.3 Hz, 1H), 5.46 (dd, J=10.3, 2.1 Hz, 1H), 4.81-4.70 (m, 1H), 4.09 (s, 2H), 3.41 (d, J=12.0 Hz, 2H), 2.82 (q, J=11.5 Hz, 2H), 2.21 (q, J=11.9 Hz, 2H), 1.94 (d, J=14.2 Hz, 2H); HRMS-ESI (m/z) calculated for C₂₁H₂₅N₂O [M+H]: 321.1961; found: 321.1962.

### 2-chloro-N-(2-methyl-5-(trifluoromethyl)phenyl)acetamide (49)

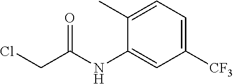 [SMILES: Cc1ccc(C(F)(F)F)cc1NC(=O)CCl]

The title compound was synthesized according to General Procedure A1 from 2-methyl-5-(trifluoromethyl)aniline (35.0 mg, 0.2 mmol, 1 equiv.). Purification of the crude product by prep. TLC (20% EtOAc/hexanes) provided the title compound as a white solid (48.2 mg, 95%). ¹H NMR (600 MHz, chloroform-d) δ 8.31 (s, 1H), 8.25 (d, J=1.9 Hz, 1H), 7.37 (dd, J=7.9, 1.8 Hz, 1H), 7.32 (d, J=7.9 Hz, 1H), 4.25 (s, 2H1), 2.36 (s, 3H); HRMS-ESI calculated for C₁₀H₁₀ClF₃NO [M+H]: 252.0397; found: 252.0397.

### 1-(5-bromoindolin-1-yl)-2-chloroethan-1-one (50)

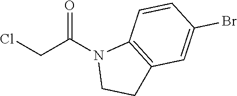 [SMILES: O=C(CCl)N1CCc2cc(Br)ccc21]

The title compound was synthesized according to General Procedure A1 from 5-bromoindoline (39.6 mg, 0.2 mmol, 1 equiv.). Purification of the crude product by prep. TLC (25% EtOAc/hexanes) provided the title compound as an off-white solid (48.6 mg, 89%). ¹H NMR (600 MHz, CDCl₃) δ 8.07 (d, J=8.4 Hz, 1H), 7.32 (d, J=8.8 Hz, 2H), 4.17 (t, J=8.6 Hz, 2H), 4.14 (s, 2H), 3.22 (t, J=8.4 Hz, 2H); HRMS-ESI (m/z) calculated for C₁₀H₁₀BrClNO [M+H]: 273.9629; found: 273.9629.

### 2-chloro-N-(quinolin-5-yl)acetamide (51)

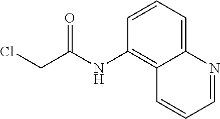 [SMILES: O=C(CCl)Nc1cccc2ncccc12]

To a stirring suspension of 5-aminoquinoline (28.8 mg, 0.2 mmol, 1 equiv.) and potassium carbonate (82.9 mg, 0.6 mmol, 3 equiv.) in anhydrous CH₂Cl₂ (3 mL) at 0° C. was added chloroacetyl chloride (24 μL, 1.5 equiv.). The reaction was allowed to slowly warm up to room temperature. After 3 hours, the mixture was filtered, washed with EtOAc (10 mL) and CH₂Cl₂ (10 mL). The solid cake was then eluted with MeOH (20 mL) and the filtrate concentrated in vacuo. The residue was taken up in 10% MeOH/CH₂Cl₂ and passed through a pad of silica to provide the title compound as an off-white solid (42.6 mg, 82%). ¹H NMR (500 MHz, CDCl₃) δ 8.96 (d, J=2.5 Hz, 1H), 8.71 (s, 1H), 8.20 (d, J=8.6 Hz, 1H), 8.04 (d, J=8.5 Hz, 1H), 7.94 (d, J=7.5 Hz, 1H), 7.74 (t, J=8.0 Hz, 1H), 7.48 (dd, J=8.5, 4.2 Hz, 1H), 4.35 (s, 2H); HRMS-ESI (m/z) calculated for C₁₁H₉ClN₂O [M+H]: 221.0476; found: 221.0477.

### 1-(4-benzylpiperidin-1-yl)prop-2-en-1-one (53)

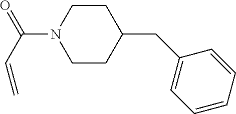 [SMILES: C=CC(=O)N1CCC(Cc2ccccc2)CC1]

Following General Procedure B, starting from 4-benzylpiperidine (1 g, 5.7 mmol, 1 equiv.), the title compound was obtained after column chromatography as a yellow oil (748 mg, 57%). ¹H NMR (500 MHz, Chloroform-d) δ 7.36 (t, J=7.4 Hz, 2H), 7.28 (t, J=7.4 Hz, 1H), 7.20 (d, J=7.1 Hz, 2H), 6.64 (dd, J=16.8, 10.6 Hz, 1H), 6.32 (dd, J=16.8, 1.9 Hz, 1H), 5.72 (dd, J=10.6, 1.9 Hz, 1H), 4.72 (d, J=12.7 Hz, 1H), 4.03 (d, J=13.0 Hz, 1H), 3.05 (t, J=12.7 Hz, 1H), 2.70-2.59 (m, 3H), 1.86 (ddp, J=14.6, 7.2, 3.5 Hz, 1H), 1.77 (m, 2H), 1.37-1.18 (m, 2H); HRMS-ESI (m/z) calculated for C₁₅H₂₀ClNO [M+H]: 230.1539; found: 230.1539.

### 2-chloro-N-((3-hydroxy-5-(hydroxymethyl)-2-methylpyridin-4-yl)methyl)acetamide (54)

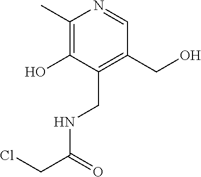 [SMILES: Cc1ncc(CO)c(CNC(=O)CCl)c1O]

To a stirred solution of pyridoxamine hydrochloride (150 mg, 0.64 mmol, 1 equiv.) in anhydrous MeOH (20 mL) at room temperature was added sodium metal (30 mg, 1.5 mmol, 2.3 equiv.), TEA (100 μL, 1 mmol, 1.6 equiv.). Chloroacetic anhydride (390 mg, 2.29 mmol, 3.5 equiv.) was added and the mixture stirred for 6 h, monitoring for completeness by TLC. After which, the reaction mixture was concentrated in vacuo. The crude product then was the purified by prep. TLC to afford the title compound as a white solid (46 mg, 30%). ¹H NMR (500 MHz, Methanol-d₄) δ 7.97 (s, 1H), 4.81 (s, 2H), 4.61 (s, 2H), 4.17 (s, 3H), 4.06 (s, 1H), 3.35 (s, 1H), 2.52 (s, 3H); HRMS-ESI (m/z) calculated for C₁₀H₁₄ClN₂O₃ [M+H]: 245.0687; found: 245.0688.

### 1-(6,7-dimethoxy-3,4-dihydroisoquinolin-2(1H)-yl)prop-2-en-1-one (56)

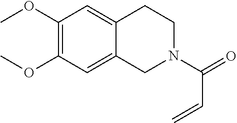 [SMILES: C=CC(=O)N1CCc2cc(OC)c(OC)cc2C1]

To a stirring suspension of the 6,7-dimethoxy-3,4-dihydroisoquinoline (1 g, 5.2 mmol, 1 equiv.) and TEA (1800 μL, 12.6 mmol, 2.5 equiv.) in anhydrous THF (10 mL) at 0° C. was added acryloyl chloride (1320 μL, 13.2 mmol, 2.6 equiv.) and the reaction was allowed to slowly warm up to room temperature. After 2 hours, the mixture was diluted with CH₂Cl₂ (2×50 mL) and washed with saturated brine (2×50 mL) and the combined organics were concentrated in vacuo. The residue was taken up in 10% MeOH/CH₂Cl₂ and purified by column chromatography to afford the title compound as a white solid (700 mg, 54%, mixture of E/Z isomers). ¹H NMR (500 MHz, Chloroform-d) δ 6.63 (m, 3H), 6.29 (d, J=16.8 Hz, 1H), 5.69 (dd, J=10.6, 1.8 Hz, 1H), 4.69 (s, 1H [major]), 4.63 (s, 0.8H [minor]), 3.82 (s, 7H), 3.73 (t, J=5.6 Hz, 1H), 2.84-2.77 (m, 2H); HRMS-ESI (m/z) calculated for C₁₄H₁₈NO₃ [M+H]: 248.128; found: 248.1281.

### 2-chloro-N-(1-(3-ethynylbenzyl)piperidin-4-yl)-N-phenylacetamide (61)

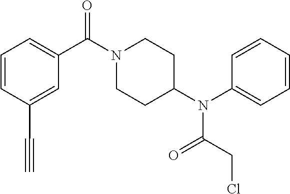 [SMILES: C#Cc1cccc(C(=O)N2CCC(N(C(=O)CCl)c3ccccc3)CC2)c1]

To an excess of neat SI-3 was added 0.7 mL of trifluoroacetic acid (0.2 M). The resulting solution was concentrated under a stream of nitrogen until no further evaporation was observed, providing the deprotected amine as its trifluoroacetate salt. The trifluoroacetate amine salt (90.6 mg, 0.25 mmol) was taken up in DMF (0.5 mL, 0.5 M) and the resulting solution was cooled to 0° C. 3-ethynyl benzoic acid (44 mg, 1.2 equiv.), HATU (113 mg, 1.2 equiv.), and Hunig's base (86 μL, 2 equiv.) were sequentially added. The reaction was stirred for 2 hours at 0° C., diluted with Et₂O, and then washed with 1 M HCl. The organic layer was dried over magnesium sulfate, concentrated, and purified by flash chromatography (gradient from 40 to 70% ethyl acetate in hexanes) to provide the title compound (87 mg, 92%). ¹H NMR (400 MHz, Chloroform-d) δ 7.51 (dd, J=9.5, 5.4 Hz, 4H), 7.43 (d, J=1.9 Hz, 1H), 7.39-7.25 (m, 2H), 7.14 (d, J=10.4 Hz, 2H), 4.86 (tt, J=15.1, 5.3 Hz, 2H), 3.72 (s, 3H), 3.19 (d, J=14.0 Hz, 1H), 3.11 (s, 1H), 2.86 (s, 1H), 1.90 (d, J=36.6 Hz, 2H), 1.38 (s, 1H), 1.24 (d, J=19.9 Hz, 1H); HRMS-ESI (m/z) calculated for C₂₂H₂₂C1N₂O₂ [M+H]: 381.1364; found: 381.1363.

### Example 2

Animal Treatment

Female DBA/1 mice (7-10 week of age) are purchased from The Jackson Laboratory (Bar Harbor, Me.), and are kept for 1 week before treatments. The animal facilities are certified by the Association for Assessment and Accreditation of Laboratory Animal Care. An illustrative compound from FIG. 1, compound A, is used for this study. The animals are injected i.p. with about 50 mg/kg of compound A (dissolved in phosphate-buffered saline) or vehicle four times weekly for 3 weeks. Four days after the last dose, mice are scarified, and splenocytes and lymph node cells are isolated for ex vivo T-cell proliferation assays.

Lymph Node and Splenic T-Cell Proliferation Assay

Splenocytes and lymph node cells obtained from the Animal Treatment study are separately pooled from three to five mice, and single-cell suspension are prepared. The cells (about 1×10⁶ cells/well) are stimulated with 10 μg/ml of compound A, and then incubated for 4 days in a 96-well plate in DMEM containing 10% fetal calf serum (FCS). During the last 16 hours, the cells are pulsed with [³H]thymidine (0.5 μCi/well), and T-cell proliferation is determined by thymidine uptake. In the lymph node proliferation assay, serum-free X-VIVO medium is used.

Electrophoresis Analysis

Splenocytes and lymph node cells obtained from the Animal Treatment study are separately pooled and centrifuged to collect the respective cell pellet. The cell pellet is subsequently lysed and resolved on a 10-12% polyacrylamide gel. Protein bands are subsequently visualized by silver staining.

### Example 3

Tumor Cell Lines and Mice

Six to eight-week female C57BL and C3H mice are purchased (Charles River Laboratories, Wilmington, Mass.). The animal facilities are certified by the Association for Assessment and Accreditation of Laboratory Animal Care.

ID8 is a clone of the MOSEC ovarian carcinoma of C57BL/6 origin. SW1 is a clone derived from the K1735 melanoma of C3H origin.

In Vivo Studies

In experiments with the ID8 ovarian carcinoma, mice (5 or 10/group) are transplanted i.p. with 3×10⁶ cells. Either 10 or 15 days later, they are injected i.p. with compound A or vehicle, which is repeated weekly for a total of 3 times. Mice are monitored daily for tumor growth, including swollen bellies indicating that they have developed ascites, and for evidence of toxicity. Tumor growth is recorded using a digital caliper. The survival of each mouse is further recorded and overall survival is calculated as mean±standard error of mean (M±SEM).

In experiments with the SW1 melanoma, 5×10⁵ cells are transplanted s.c. on the right flank, When the mice have developed tumors of about 4-5 mm in mean diameter, they are randomized into treatment group and control group; with either compound A or vehicle injected i.p., respectively, at weekly intervals for a total of 3 times. Mice are monitored daily for evidence of toxicity. Tumor diameters are measured twice/week using a digital caliper and tumor surfaces are calculated. Overall survival is also recorded.

### Example 4

Phase 1 Clinical Trial

Purpose: this clinical trial is to assess the safety and tolerability of administration of compound A in combination with low-dose cytokines (IL-2 and IFN-alpha) in patients with metastatic or refractory cancer.

Study Type: Interventional

Study Design: Allocation: Non-Randomized

Intervention Model: Single Group Assignment

Masking: Open Label

Primary Purpose: Treatment

Primary Outcome Measures:
Safety [Time Frame: Initial dose of study therapy through 30 days post last dose of study therapy]
Tolerability [Time Frame: Initial dose of study therapy through 30 days post last dose of study therapy]
Anti-tumor Activity [Time Frame: From initial dose of study therapy to disease progression]

Eligibility

Ages Eligible for Study: 18 Years and older (Adult, Senior)

Sexes Eligible for Study: All

Accepts Healthy Volunteers: No

Criteria

Inclusion Criteria:
Have a histologically confirmed diagnosis of metastatic or refractory cancer for which there are no effective standard therapeutic options available;
Have signed an Institutional Review Board (IRB) approved informed consent form (ICF) prior to performing any study evaluation/procedures;
Be > or =18 years of age and women must either be 1) not of childbearing potential or 2) have a negative serum pregnancy test within 7 days prior to commencing treatment. Patients are considered not of childbearing potential if they are surgically sterile (they have undergone a hysterectomy, bilateral tubal ligation or bilateral oophorectomy) or they are postmenopausal (12 consecutive months of amenorrhea [lack of menstruation]);
(If applicable) Have completed prior cytotoxic chemotherapy, radiotherapy or immunotherapy or experimental therapy > or =30 days prior to the study enrollment, and recovered form associated toxicities;
Have an Eastern Cooperative Oncology Group (ECOG) score of < or =2, and an anticipated life expectancy of at least 6 months;
Have adequate hematologic function, as defined by an absolute or calculated neutrophil count > or =1500/microL, platelet count > or =100000/microL, lymphocyte count > or =500/microL, and hemoglobin level > or =10 g/dL. Patients may not receive prophylactic transfusion in order to qualify for trial eligibility;
Have adequate renal function, as defined by a documented serum creatinine of < or =2.0 mg/dL. Greater than “1+” proteinuria will require microscope evaluation and the results discussed with the medical monitor prior to patient enrollment; or if serum creatine is >2.0, patient must have an actual or calculated 24-hour creatinine clearance of >60 mL/min and no obvious evidence of concurrent medullary cystic disease or obstructive uropathy;
Have adequate hepatic function, as defined by a total bilirubin level < or =1.5× upper limit of normal (ULN) and alkaline phosphatase, aspartate transaminase (AST), and alanine transaminase (ALT) levels < or =2.5×ULN. If alkaline phosphatase is outside of these parameters and is due to bone metastases (as verified by the assessment of isoenzymes), then the patient is eligible.

Exclusion Criteria:
Have a history of severe hypersensitivity (grade 3-4 allergic reaction) to fluorescein or any drug, radiologic contrast agent, insect bite, food, cytokines, or any other agent; or have received fluorescein within 30 days of the study;
Have medical conditions that preclude the use of IL-2 or IFN-alpha. These conditions include but are not limited to, diabetes mellitus with a history of progression to diabetic ketoacidosis, history of severe coagulation disorder, psoriasis, sarcoidosis, retinal hemorrhage, symptomatic pulmonary disease, heart failure (> or =New York Heart Association NYHA class II), or transplant requiring immunosuppressive therapy;
Be pregnant or breast-feeding;
Be currently receiving an experimental drug, or used an experimental device within 30 days of study entry;
Be currently undergoing chemotherapy, anticancer hormonal therapy, and/or therapy with immunosuppressant agents;
Have any concomitant malignancy with the exception of basal cell or squamous cell carcinoma of skin;
Have radiographically documented evidence of current brain metastases, a history of stem cell transplant, immunodeficiency, and/or a medical or psychiatric illness (that in the investigator's opinion, would prevent adequate compliance with study therapy or evaluation of the endpoints).

### Example 5

Tables 1-5 illustrate exemplary lists of cysteine-containing polypeptides.

Table 1 illustrates an exemplary list of liganded cysteines which are identified from isoTOP-ABPP experiments performed in cell lysates (in vitro). Table 1 further shows the accession number (or the protein identifier) of the protein, cysteine residue number, and an illustrative peptide fragment containing the cysteine of interest (denoted by C*). For example, P23396 (row 2, col 1) is the accession number (or protein identifier) of RPS3 40S ribosomal protein S3. C97 (row 2, col 1) is the cysteine residue number of interest. The peptide fragment: R.GLC*AIAQAESLR.Y (SEQ ID NO: 1) is an illustrative peptide fragment of RPS3 40S ribosomal protein S3 containing the cysteine residue C97 and is denoted by C*.

TABLE 1

| | | | SEQ ID |
|---|---|---|---|
| Identifier | Protein Name | Illustrative Cysteine Fragment | NO: |
| P23396_ | RPS3 40S ribosomal protein S3 | R.GLC*AIAQAESLR.Y | 1 |
| C97 | | | |
| P68366_ | TUBA4A Tubulin alpha-4A chain | K.AYHEQLSVAEITNAC*FEPANQMVK.C | 2 |
| C295 | | | |
| P68366_ | TUBA4A Tubulin alpha-4A chain | K.RSIQFVDWC*PTGFK.V | 3 |
| C347 | | | |
| Q71U36_ | TUBA1A Tubulin alpha-1A chain | K.RTIQFVDWC*PTGFK.V | 4 |
| C347 | | | |
| Q71U36_ | TUBA1A Tubulin alpha-1A chain | R.AVC*MLSNTTAIAEAWAR.L | 5 |
| C376 | | | |
| Q7L1Q6_ | BZW1 Basic leucine zipper and W2 | K.ERFDPTQFQDC*IIQGLTETGTDLEAVAK. | 6 |
| C35 | domain-containing protein | F | |
| P04406_ | GAPDH Glyceraldehyde-3-phosphate | K.IISNASC*TTNCLAPLAK.V | 7 |
| C152 | dehydrogenase | | |
| Q99873_ | PRMT1 Protein arginine N- | K.VIGIEC*SSISDYAVK.I | 8 |
| C109 | methyltransferase 1 | | |
| Q15365_ | PCBP1 Poly(rC)-binding protein 1 | R.LVVPATQC*GSLIGK.G | 9 |
| C109 | | | |
| Q13526_ | PIN1 Peptidyl-prolyl cis-trans | K.IKSGEEDFESLASQFSDC*SSAK.A | 10 |
| C113 | isomerase NIMA-interacting 1 | | |
| Q9Y266_ | NUDC Nuclear migration protein | R.WTQTLSELDLAVPFC*VNFR.L | 11 |
| C188 | nudC | | |
| Q9Y696_ | CLIC4 Chloride intracellular channel | K.AGSDGESIGNC*PFSQR.L | 12 |
| C35 | protein 4 | | |
| P24752_ | ACAT1 Acetyl-CoA | R.QAVLGAGLPISTPC*TTINK.V | 13 |
| C119 | acetyltransferase, mitochondrial | | |
| P10809_ | HSPD1 60 kDa heat shock protein, | R.AAVEEGIVLGGGC*ALLR.C | 14 |
| C442 | mitochondrial | | |
| P68366_ | TUBA4A Tubulin alpha-4A chain | K.YMAC*CLLYR.G | 15 |
| C315 | | | |
| P09211_ | GSTP1 Glutathione S-transferase P | K.ASC*LYGQLPK.F | 16 |
| C48 | | | |
| Q86SX6_ | GLRX5 Glutaredoxin-related protein | K.GTPEQPQC*GFSNAVVQILR.L | 17 |
| C67 | 5, mitochondrial | | |
| P13639_ | EEF2 Elongation factor 2 | K.STLTDSLVC*K.A | 18 |
| C41 | | | |
| O14980_ | XPO1 Exportin-1 | K.LDINLLDNVVNC*LYHGEGAQQR.M | 19 |
| C34 | | | |
| Q15365_ | PCBP1 Poly(rC)-binding protein 1 | R.VMTIPYQPMPASSPVIC*AGGQDR.C | 20 |
| C194 | | | |
| Q99832_ | CCT7 T-complex protein 1 subunit eta | K.EGTDSSQGIPQLVSNISAC*QVIAEAVR.T | 21 |
| C29 | | | |
| P62280_ | RPS11 40S ribosomal protein S11l | K.C*PFTGNVSIR.G | 22 |
| C60 | | | |
| P78417_ | GSTO1 Glutathione S-transferase | R.FC*PFAER.T | 23 |
| C32 | omega-1 | | |
| P24752_ | ACAT1 Acetyl-CoA | K.IHMGSC*AENTAK.K | 24 |
| C196 | acetyltransferase, mitochondrial | | |
| P10599_ | TXN Thioredoxin | K.LVVVDFSATWC*GPCK.M | 25 |
| C32 | | | |
| P50990_ | CCT8 T-complex protein 1 subunit | KIAVYSC*PFDGMITETK.G | 26 |
| C244 | theta | | |
| P13667_ | PDIA4 Protein disulfide-isomerase A4 | K.ENFDEVVNDADIILVEFYAPWC*GHCK.K | 27 |
| C206 | | | |
| P55735_ | SEC13 Protein SEC13 homolog | R.FASGGC*DNLIK.L | 28 |
| C187 | | | |
| Q15185_ | PTGES3 Prostaglandin E synthase 3 | K.HLNEIDLFHC*IDPNDSK.H | 29 |
| C58 | | | |
| P55263_ | ADK Adenosine kinase | R.TGC*TFPEKPDFH | 30 |
| C353 | | | |
| P46782_ | RPS5 40S ribosomal protein S5 | K.AQC*PIVER.L | 31 |
| C66 | | | |
| Q99439_ | CNN2 Calponin-2 | K.AGQC*VIGLQMGTNK.C | 32 |
| C164 | | | |
| P13639_ | EEF2 Elongation factor 2 | R.VTDGALVVVDCVSGVC*VQTETVLR.Q | 33 |
| C136 | | | |
| Q2TAA2_ | IAH1 Isoamyl acetate-hydrolyzing | R.VILITPTPLC*ETAWEEQCIIQGCK.L | 34 |
| C137 | esterase 1 homolog | | |
| P30044_ | PRDX5 Peroxiredoxin-5, | K.ALNVEPDGTGLTC*SLAPNIISQL | 35 |
| C204 | mitochondrial | | |
| P12268_ | IMPDH2 Inosine-5-monophosphate | R.HGFC*GIPITDTGR.M | 36 |
| C140 | dehydrogenase 2 | | |
| Q99714_ | HSD17810 3-hydroxyacyl-CoA | K.VC*NFLASQVPFPSR.L | 37 |
| C214 | dehydrogenase type-2 | | |
| Q9NQR4_ | NIT2 Omega-amidase NIT2 | R.VGLGIC*YDMR.F | 38 |
| C153 | | | |
| P15121_ | AICR1B1 Aldose reductase | R.VC*ALLSCTSHK.D | 39 |
| C299 | | | |
| Q6UB35_ | MTHFD1L Monofunctional C1 - | K.IDRYTQQGFGNLPIC*MAK.T | 40 |
| C906 | tetrahydrofolate synthase, | | |
| | mitochondrial | | |
| P07437_ | TUBB Tubulin beta chain | K.LTTPTYGDLNHLVSATMSGVTTC*LR.F | 41 |
| C239 | | | |
| Q15084_ | PDIA6 Protein disulfide-isomerase A6 | R.EVIQSDSLWLVEFYAPWC*GHCQR.L | 42 |
| C55 | | | |
| P14618_ | PKM Pyruvate kinase isozymes | K.C*CSGAIIVLTK.S | 43 |
| C423 | M1/M2 | | |
| P62266_ | RPS23 40S ribosomal protein S23 | K.ITAFVPNDGC*LNFIEENDEVLVAGFGR.K | 44 |
| C90 | | | |
| Q71U36_ | TUBA1A Tubulin alpha-1A chain | MRECISIHVGQAGVQIGNAC*WELYCLEHG | 45 |
| C20 | | IQPDGQMPSDK.T | |
| O95336_ | PGLS 6-phosphogluconolactonase | R.AAC*CLAGAR.A | 46 |
| C32 | | | |
| P24752_ | ACAT1 Acetyl-CoA | K.QGEYGLASIC*NGGGGASAMLIQK.L | 47 |
| C413 | acetyltransferase, mitochondrial | | |
| P62701_ | RPS4X 40S ribosomal protein S4, X | K.LREC*LPLIIFLR.N | 48 |
| C41 | isoform | | |
| P62829_ | RPL23 60S ribosomal protein L23 | R.ISLGLPVGAVINC*ADNTGAK.N | 49 |
| C28 | | | |
| Q71U36_ | TUBA1A Tubulin alpha-1A chain | R.ECISTHVGQAGVQIGNACWELYC*LEHGI | 50 |
| C25 | | QPDGQMPSDK.T | |
| Q96HE7_ | ERO1L ERO1-like alpha | K.HDDSSDNFC*EADDIQSPEAEYVDLLLNP | 51 |
| C166 | protein | ER.Y | |
| Q96HE7_ | ERO1L ERO1-like protein alpha | K.RPLNPLASGQGTSEENTFYSWLEGLC*VE | 52 |
| C241 | | K.R | |
| Q9BRA2_ | TXNDC17 Thioredoxin domain- | K.SWC*PDCVQAEPVVR.E | 53 |
| C43 | containing protein 17 | | |
| P13667_ | PDIA4 Protein disulfide-isomerase A4 | K.DVLIEFYAPWC*GHCK.Q | 54 |
| C555 | | | |
| P30101_ | PDIA3 Protein disulfide-isomerase A3 | K.DVLIEFYAPWC*GHCK.N | 55 |
| C406 | | | |
| P07437_ | TUBB Tubulin beta chain | K.TAVC*DIPPR.G | 56 |
| C354 | | | |
| P83731_ | RPL24 60S ribosomal protein L24 | K.C*ESAFLSK.R | 57 |
| C36 | | | |
| Q9ULV4_ | CORO1C Coronin-1C | K.C*DLISIPK.K | 58 |
| C420 | | | |
| P42677_ | RPS27 40S ribosomal protein S27 | R.LTEGC*SFR.R | 59 |
| C77 | | | |
| O00299_ | CLIC1 Chloride intracellular channel | KIGNC*PFSQR.L | 60 |
| C24 | protein 1 | | |
| P49458_ | SRP9 Signal recognition particle 9 | K.VTDDLVC*LVYK.T | 61 |
| C48 | kDa protein | | |
| P42765_ | ACAA2 3-ketoacyl-CoA thiolase, | R.LC*GSGFQSIVNGCQEICVK.E | 62 |
| C92 | mitochondrial | | |
| Q15233_ | NONO Non-POU domain-containing | R.FAC*HSASLTVR.N | 63 |
| C145 | octamer-binding protein | | |
| P50570_ | DNM2 Dynamin-2 | K.LQDAFSSIGQSC*HLDLPQIAVVGGQSAG | 64 |
| C27 | | K.S | |
| P25205_ | MCM3 DNA replication licensing | R.TLTSC*FLSCVVCVEGIVTK.C | 65 |
| C119 | factor MCM3 | | |
| Q5TFE4_ | NT5DC1 5-nucleotidase domain- | K.HFLSDTGMAC*R.S | 66 |
| C119 | containing protein 1 | | |
| Q99439_ | CNN2 Calponin-2 | K.C*ASQVGMTAPGTR.R | 67 |
| C215 | | | |
| Q06323_ | PSME1 Proteasome activator complex | K.VDVFREDLC*TK.T | 68 |
| C22 | subunit 1 | | |
| P49591_ | SARS Serine--tRNA ligase, | R.TIC*AILENYQTEK.G | 69 |
| C438 | cytoplasmic | | |
| Q96RS6_ | NUDCD1 NudC domain-containing | R.DSAQC*AAIAER.L | 70 |
| C376 | protein 1 | | |
| Q71U36_ | TUBA1A Tubulin alpha-1A chain | K.LADQC*TGLQGFLVFHSFGGGTGSGFTSL | 71 |
| C129 | | LMER.L | |
| Q9BXJ9_ | NAA15 N-alpha-acetyltransferase 15, | R.LFNTAVC*ESK.D | 72 |
| C721 | NatA auxiliary subunit | | |
| P33240_ | CSTF2 Cleavage stimulation factor | K.LC*VQNSPQEAR.N | 73 |
| C150 | subunit 2 | | |
| Q13155_ | AIMP2 Aminoacyl tRNA synthase | K.SPWLAGNELTVADVVLWSVLQQIGGC*S | 74 |
| C291 | complex-interacting multif | VTVPANVQR.W | |
| O75663_ | T1PRL TIP41-like protein | K.VAC*AEEWQESR.T | 75 |
| C87 | | | |
| P63244_ | GNB2L1 Guanine nucleotide-binding | K.VWNLANC*K.L | 76 |
| C182 | protein subunit beta-2-like 1 | | |
| Q7L2H7_ | EIF3M Eukaryotic translation | K.VAASC*GAIQYIPTELDQVR.K | 77 |
| C134 | initiation factor 3 subunit | | |
| P49327_ | FASN Fatty acid synthase | K.LTPGC*EAEAETEAICFFVQQFTDMEHNR. | 78 |
| C2359 | | V | |
| P42166_ | TMPO Lamina-associated polypeptide | K.VDDEILGFISEATPLGGIQAASTESC*NQQ | 79 |
| C561 | 2, isoform alpha | LDLALCR.A | |
| P24752_ | ACAT1 Acetyl-CoA | K.VC*ASGMK.A | 80 |
| C126 | acetyltransferase, mitochondrial | | |
| P36578_ | RPL4 60S ribosomal protein L4 | R.SGQGAFGNMC*R.G | 81 |
| C96 | | | |
| Q9NXG2_ | THUMPD1 THUMP domain- | R.RC*DAGGPR.Q | 82 |
| C31 | containing protein 1 | | |
| P07237_ | P4HB Protein disulfide-isomerase | K.KNVFVEFYAPWC*GHCK.Q | 83 |
| C397 | | | |
| P27707_ | DCK Deoxycytidine kinase | R.SC*PSFSASSEGTR.I | 84 |
| C9 | | | |
| O00303_ | EIF3F Eukaryotic translation initiation | K.TC*FSPNR.V | 85 |
| C256 | factor 3 subunit | | |
| Q9UNH7_ | SNX6 Sorting nexin-6 | K.SAADDYNRIGSSLYALGTQDSTDIC*KFF | 86 |
| C264 | | LK.V | |
| Q15084_ | PDIA6 Protein disulfide-isomerase A6 | K.DVIELTDDSFDKNVLDSEDVWMVEFYAP | 87 |
| C190 | | WC*GHCK.N | |
| O43390_ | HNRNPR Heterogeneous nuclear | K.SAFLC*GVMK.T | 88 |
| C99 | ribonucleoprotein R | | |
| Q96JB2_ | COG3 Conserved oligomeric Golgi | K.AAAENLPVPAELPIEDLC*SLTSQSLPIELT | 89 |
| C65 | complex subunit 3 | SVVPESTEDILLK.G | |
| P46109_ | CRKL Crk-Like protein | K.RVPC*AYDK.T | 90 |
| C249 | | | |
| P62888_ | RPL30 60S ribosomal protein L30 | R.VC*TLAIIDPGDSDIIR.S | 91 |
| C92 | | | |
| Q9NUY8_ | TBC1D23 TBC1 domain family | K.FLENTPSSLNIEDIEDLFSLAQYYC*SK.T | 92 |
| C283 | member 23 | | |
| Q09161_ | NCBP1 Nuclear cap-binding protein | K.SAC*SLESNLEGLAGVLEADLPNYK.S | 93 |
| C44 | subunit 1 | | |
| Q9UJW0_ | DCTN4 Dynactin subunit 4 | R.LLQPDFQPVC*ASQLYPR.H | 94 |
| C258 | | | |
| O15294_ | OGT UDP-N-acetylglucosamine- | K.C*PDGGDNADSSNTALNMPVIPMNTIAE | 95 |
| C758 | peptide N-acetylglucosaminyl | AVIEMINR.G | |
| | transferase | | |
| P38646_ | HSPA9 Stress-70 protein, | K.AKC*ELSSSVQTDINLPYLTMDSSGPK.H | 96 |
| C317 | mitochondrial | | |
| Q9HA64_ | FN3KRP Ketosamine-3-kinase | R.ATGHSGGGC*ISQGR.S | 97 |
| C24 | | | |
| Q9Y6G9_ | DYNCILI1 Cytoplasmic dynein 1 | R.VGSFGSSPPGLSSTYTGGPLGNEIASGNG | 98 |
| C51 | light intermediate chain 1 | GAAAGDDEDGQNLWSC*ILSEVSTR.S | |
| P52597_ | HNRNPF Heterogeneous nuclear | R.DLSYC*LSGMYDHR.Y | 99 |
| C267 | ribonucleoprotein F | | |
| Q99497_ | PARK7 Protein DJ-1 | K.GLIAAIC*AGPTALLAHEIGFGSK.V | 100 |
| C106 | | | |
| P57764_ | GSDMD Gasdermin-D | R.C*LHNFLTDGVPAEGAFTEDFQGLR.A | 101 |
| C268 | | | |
| A6NDG6_ | PGP Phosphoglycolate phosphatase | K.NNQESDC*VSK.K | 102 |
| C297 | | | |
| Q14566_ | MCM6 DNA replication licensing | R.LVFLAC*CVAPTNPR.F | 103 |
| C301 | factor MCM6 | | |
| Q00610_ | CLTC Clathrin heavy chain 1 | R.IHEGC*EEPATHNALAK.I | 104 |
| C870 | | | |
| Q96T76_ | MMS19 MMS19 nucleotide excision | R.LMGLLSDPELGPAAADGFSLLMSDC*TD | 105 |
| C848 | repair protein homolog | VLTR.A | |
| P61158_ | ACTR3 Actin-related protein 3 | R.YSYVC*PDLVK.E | 106 |
| C235 | | | |
| P23528_ | CFL1 Cofilin-1 | K.MLPDKDC*R.Y | 107 |
| C80 | | | |
| Q9NQ88_ | TIGAR Fructose-2,6-bisphosphatase | K.EADQKEQFSQGSPSNC*LETSLAEIFPLGK. | 108 |
| C161 | TIGAR | N | |
| Q9UKF6_ | CPSF3 Cleavage and polyadenylation | R.NFNYHILSPC*DLSNYTDLAMSTVK.Q | 109 |
| C498 | specificity factor subunit | | |
| Q8TAQ2_ | SMARCC2 SWI/SNF complex | R.PNIFLC*PEIEPK.L | 110 |
| C145 | subunit SMARCC2 | | |
| P68036_ | UBE2L3 Ubiquitin-conjugating | K.GQVC*LPVISAENWKPATK.T | 111 |
| C86 | enzyme E2 L3 | | |
| Q9Y3A3_ | MOB4 MOB-like protein phocein | R.HTLDGAAC*LLNSNK.Y | 112 |
| C134 | | | |
| P49588_ | AARS Alanine-tRNA ligase, | K.C*LSVMEAK.V | 113 |
| C773 | cytoplasmic | | |
| Q961J6_ | GMPPA Mannose-1 -phosphate | K.LLPAITILGC*R.V | 114 |
| C389 | guanyltransferase alpha | | |
| P27635_ | RPL10 60S ribosomal protein L10 | K.MLSC*AGADR.L | 115 |
| C105 | | | |
| O15145_ | ARPC3 Actin-related protein 2/3 | K.WWTC*FVK.R | 116 |
| C162 | complex subunit 3 | | |
| P48643_ | CCT5 T-complex protein 1 subunit | K.IAILTC*PFEPPKPK.T | 117 |
| C253 | epsilon | | |
| P11586_ | MTHFD1 C-1-tetrahydrofolate | R.ASVGAGFLYPLVGTMSTMPGLPTRPC*F | 118 |
| C918 | synthase, cytoplasmic | YDIDLDPETEQVNGLF | |
| Q96GX2_ | ATXN7L3B Putative ataxin-7-like | R.LPLC*SLPGEPGNGPDQQLQRS | 119 |
| C75 | protein 3B | | |
| P17987_ | TCP1 T-complex protein 1 subunit | K.VLC*ELADLQDK.E | 120 |
| C76 | alpha | | |
| Q9NYL9_ | TMOD3 Tropomodulin-3 | K.VSLDPELEEALTSASDTELC*DLAAILGM | 121 |
| C132 | | HNLITNTK.F | |
| O00429_ | DNM1L Dynkamin-1-like protein | R.IC*YIFHETFGR.T | 122 |
| C367 | | | |
| Q13155_ | AIMP2 Aminoacyl tRNA synthase | R.VELPTC*MYR.L | 123 |
| C23 | complex-interacting multif | | |
| P30101_ | PDIA3 Protein disulfide-isomerase A3 | R.ISDTGSAGLMLVEFFAPWC*GHCK.R | 124 |
| C571 | | | |
| Q9ULA0_ | DNPEP Aspartyl aminopeptidase | K.C*PTSGR.L | 125 |
| C144 | | | |
| Q9BWD1_ | ACAT2 Acetyl-CoA | R.QASVGAGIPYSVPAWSCQMIC*GSGLK.A | 126 |
| C92 | acetyltransferase, cytosolic | | |
| Q8NBS9_ | TXNDC5 Thioredoxin domain- | K.FYAPWC*GHCK.T | 127 |
| C350 | containing protein 5 | | |
| P68366_ | TUBA4A Tubulin alpha-4A chain | K.TIGGGDDSFTTFFC*ETGAGK.H | 128 |
| C54 | | | |
| P04183_ | TK1 Thymidine kinase, cytosolic | R.YSSSFC*THDR.N | 129 |
| C66 | | | |
| Q15021_ | NCAPD2 Condensin complex subunit | K.NAIQLLASFLANNPFSC*K.L | 130 |
| C439 | 1 | | |
| QSWUM4_ | PDCD6IP Programmed cell death 6- | K.HC*IMQANAEYHQSILAK.Q | 131 |
| C250 | interacting protein | | |
| Q96F86_ | EDC3 Enhancer of mRNA-decapping | K.DLPTSPVDLVINCLDC*PENVFLR.D | 132 |
| C413 | protein 3 | | |
| P35754_ | GLRX Glutaredoxin-1 | K.VVVFIKPTC*PYCR.R | 133 |
| C23 | | | |
| Q9NQ88_ | TIGAR Fructose-2,6-bisphosphatase | R.EEC*PVFTPPGGETLDQVK.M | 134 |
| C114 | TIGAR | | |
| P12268_ | IMPDH2 Inosine-5-monophosphate | R.VGMGSGSIC*ITQEVLACGRPQATAVYK. | 135 |
| C331 | dehydrogenase 2 | V | |
| P25705_ | ATP5A1 ATP synthase subunit alpha, | K.TIVVSATASDAAPLQYLAPYSGC*SMGE | 136 |
| C294 | mitochondrial | YFR.D | |
| P15374_ | UCHL3 Ubiquitin carboxyl-terminal | K.QTISNAC*GTIGLIHAIANNK.D | 137 |
| C95 | hydrolase isozyme L3 | | |
| P83731_ | RPL24 60S ribosomal protein L24 | K.VELC*SFSGYK.I | 138 |
| C6 | | | |
| Q9NX24_ | NHP2 H/ACA ribonucleoprotein | K.IKADPDGPEAQAEAC*SGER.T | 139 |
| C18 | complex subunit 2 | | |
| Q13418_ | ILK Integrin-linked protein lcinase | FSFQCPGRK.FSFQC*PGR.M | 140 |
| C346 | | | |
| Q13428_ | TCOF1 Treacle protein | K.C*FLAQPVTLLDIYTHWQQTSELGR.K | 141 |
| C38 | | | |
| P24941_ | CDK2 Cyclin-dependent kinase 2 | R.APEILLGC*K.Y | 142 |
| C177 | | | |
| P43686_ | PSMC4 26S protease regulatory | R.GVLMYGPPGC*GK.T | 143 |
| C210 | subunit 6B | | |
| Q9BTA9_ | WAC WW domain-containing adapter | R.STC*SLTPALAAHFSENLIK.H | 144 |
| C553 | protein with coiled-coil | | |
| O14929_ | HAT1 Histone acetyltransferase type | K.VDENFDC*VEADDVEGK.I | 145 |
| C101 | B catalytic subunit | | |
| O75521_ | ECI2 Enoyl-CoA delta isomerase 2, | R.WLSDEC*TNAVVNFLSR.K | 146 |
| C380 | mitochondrial | | |
| P11586_ | MTHFD1 C-1-tetrahydrofolate | K.QGFGNLPIC*MAK.T | 147 |
| C863 | synthase, cytoplasmic | | |
| P27707_ | DCK Deoxycytidine kinase | K.QLC*EDWEVVPEPVAR.W | 148 |
| C45 | | | |
| A0AVT1_ | UBA6 Ubiquitin-like modifier- | R.KPNVGC*QQDSEELLK.L | 149 |
| C347 | activating enzyme 6 | | |
| Q96AB3_ | ISOC2 Isochorismatase domain- | R.SVLLCGIEAQAC*ILNTTLDLLDR.G | 150 |
| C114 | containing protein 2, mitochondrial | | |
| Q14203_ | DCTN1 Dynactin subunit 1 | K.VTFSC*AAGFGQR.H | 151 |
| C1252 | | | |
| Q9BTE3_ | MCMBP Mini-chromosome | R.DASALLDPMEC*TDTAEEQR.V | 152 |
| C287 | maintenance complex-binding protein | | |
| O95373_ | IPO7 Importin-7 | R.GIDQC*IPLFVEAALER.L | 153 |
| C757 | | | |
| P22061_ | PCMT1 Protein-L-isoaspartate(D- | R.MVGC*TGK.V | 154 |
| C102 | aspartate) O-methyltransferase | | |
| P55060_ | CSE1L Exportin-2 | K.KIC*AVGITK.L | 155 |
| C842 | | | |
| Q9BX19_ | NAA15 N-alpha-acetyltransferase 15, | K.GC*PPVFNTLR.S | 156 |
| CC322 | NatA auxiliary subunit | | |
| P62333_ | PSMC6 26S protease regulatory | K.GC*LLYGPPGTGK.T | 157 |
| C170 | subunit 10B | | |
| Q15398_ | DLGAP5 Disks large-associated | R.YRPDMPC*FLLSNQNAVK.A | 158 |
| C129 | protein 5 | | |
| Q9BVC5_ | C2orf49 Ashwin | R.SC*TDSELLLHPELLSQEFLLLTLEQK.N | 159 |
| C10 | | | |
| P62280_ | RPS11 40S ribosomal protein S11 | K.NMSVHLSPC*FR.D | 160 |
| C116 | | | |
| P42224_ | STAT1 Signal transducer and | R.QQSACIGGPPNAC*LDQLQNWFTIVAESL | 161 |
| C255 | activator of transcription 1 | QQVR.Q | |
| Q96RN5_ | MED15 Mediator of RNA polymerase | K.QQYLC*QPLLDAVLANIR.S | 162 |
| C618 | II transcription subunit | | |
| Q6UB35_ | MTHFD1L Monofunctional C1- | R.ASIGAGFIYPLVGTMSTMPGLPTRPC*FY | 163 |
| C961 | tetrahydrofolate synthase, | DIDLDTETEQVK.G | |
| | mitochondrial | | |
| P23919_ | DTYMK Thymidylate kinase | R.C*FHQLMK.D | 164 |
| C163 | | | |
| Q92616_ | GCN1L1 Translational activator | K.GMGESC*FEDLLPWLMETLTYEQSSVDR. | 165 |
| C1692 | GCN1 | S | |
| P42166_ | TMPO Lamina-associated polypeptide | K.SGIQPLC*PER.S | 166 |
| C341 | 2, isoform alpha | | |
| O95571_ | ETHE1 Protein ETHE1, | R.TDFQQGC*AK.T | 167 |
| C170 | mitochondrial | | |
| O14777_ | NDC80 Kinetochore protein NDC80 | K.FNPEAGANC*LVK.Y | 168 |
| C449 | homolog | | |
| P36542_ | ATP5C1 ATP synthase subunit | R.GLC*GAIHSSIAK.Q | 169 |
| C103 | gamma, mitochondrial | | |
| Q9Y3D0_ | FAM96B Mitotic spindle-associated | R.VQVSDPESTVAVAFTPTIPHC*SMATLIGL | 170 |
| C93 | MMXD complex subunit Mi | SIK.V | |
| P54136_ | RARS Arginine-tRNA ligase, | K.NCGC*LGASPNLEQLQEENLK.L | 171 |
| C34 | cytoplasmic | | |
| Q96T76_ | MMS19 MMS19 nucleotide excision | R.YHPLSSC*LTAR.L | 172 |
| C819 | repair protein homolog | | |
| Q8TBC4_ | UBA3 NEDD8-activating enzyme E1 | R.VILPGMTACIECTLELYPPQVNFPMC*TIA | 173 |
| C237 | catalytic subunit | SMPR.L | |
| Q13573_ | SNW1 SNW domain-containing | K.IPPC*ISNWK.N | 174 |
| C250 | protein 1 | | |
| P46734_ | MAP2K3 Dual specificity mitogen- | K.MC*DFGISGYLVDSVAK.T | 175 |
| C207 | activated protein kinase | | |
| P34932_ | HSPA4 Heat shock 70 kDa protein 4 | R.AGGIETIANEYSDRC*TPACISFGPK.N | 176 |
| C34 | | | |
| Q9UBR2_ | CTSZ Cathepsin Z | R.NQHIPQYCGSC*WAHASTSAMADR.I | 177 |
| C92 | | | |
| Q16763_ | UBE2S Ubiquitin-conjugating enzyme | K.C*LLIRPNPESALNEEAGR.L | 178 |
| C118 | E2 S | | |
| Q06203_ | PPAT | K.C*ELENCQPFVVETLHGKI | 179 |
| C100 | Amidophosphoribosyltransferase | | |
| P51649_ | ALDH5A1 Succinate-semialdehyde | R.NTGQTC*VCSNQFLVQR.G | 180 |
| C340 | dehydrogenase, mitochondrial | | |
| P43246_ | MSH2 DNA mismatch repair protein | K.KGVC*DQSFGIHVAELANFPK.H | 181 |
| C822 | Msh2 | | |
| P27708_ | CAD CAD protein | K.AQILVLTYPLIGNYGIPPDEMDEFGLC*K. | 182 |
| C73 | | W | |
| Q16822_ | PCK2 Phosphoenolpyruvate | R.YVAAAFPSAC*GK.T | 183 |
| C306 | carboxylcinase | | |
| Q01813_ | PFKP 6-phosphofructokinase type C | R.NESC*SENYTTDFIYQLYSEEGK.G | 184 |
| C641 | | | |
| Q96CM8_ | ACSF2 Acyl-CoA synthetase family | R.MVSTPIGGLSYVQGC*TK.K | 185 |
| C64 | member 2, mitochondrial | | |
| Q9BUK6_ | MSTO1 Protein misato homolog 1 | K.VVTAGAIIPFPLAPGQSLPDSLMQFGGAT | 186 |
| C403 | | PWTPLSAC*GEPSGTR.C | |
| P22234_ | PA1CS Multifunctional protein ADE2 | R.LPSGLGC*STVLSPEGSAQFAAQIFGLSNH | 187 |
| C374 | | LVWSK.L | |
| Q6ICB0_ | DESI1 Desumoylating isopeptidase 1 | R.GEAYNLFEHNC*NTFSNEVAQFLTGRK | 188 |
| C108 | | | |
| P62333_ | PSMC6 26S protease regulatory | R.AVASQLDC*NFLK.V | 189 |
| C193 | subunit 10B | | |
| Q8IUR0_ | TRAPPC5 Trafficking protein particle | K.ENSTLNC*ASFTAGIVEAVLTHSGFPAK.V | 190 |
| C139 | complex subunit 5 | | |
| Q01813_ | PFKP 6-phosphofructokinase type C | K.YAYLNVVGMVGSIDNDFC*GTDMTIGTD | 191 |
| C179 | | SALHR.I | |
| P38606_ | ATP6V1A V-type proton ATPase | K.WDFTPC*K.N | 192 |
| C138 | catalytic subunit A | | |
| P82932_ | MRPS6 28S ribosomal protein S6, | K.EC*EGIVPVPLAEK.L | 193 |
| C105 | mitochondrial | | |
| Q9NS86_ | LANCL2 LanC-like protein 2 | R.SVVC*QESDLPDELLYGR.A | 194 |
| C187 | | | |
| Q9ULV4_ | CORO1C Coronin-1C | K.SIKDTIC*NQDER.I | 195 |
| C456 | | | |
| P09110_ | ACAA1 3-ketoacyl-CoA thiolase, | K.VNPLGGAVALGHPLGC*TGAR.Q | 196 |
| C381 | peroxisomal | | |
| Q9HAV7_ | GRPEL1 GrpE protein homolog 1, | K.ATQC*VPKEEIKDDNPHLK.N | 197 |
| C124 | mitochondrial | | |
| Q14738_ | PPP2R5D Serine/threonine-protein | K.C*TAKPSSSGK.D | 198 |
| C17 | phosphatase 2A 56 kDa regulatory | | |
| | subunit delta isoform | | |
| Q99757_ | TXN2 Thioredoxin, mitochondrial | R.VVNSETPVVVDFHAQWC*GPCK.I | 199 |
| C90 | | | |
| P51570_ | GALK1 Galactokinase | R.AQVCQQAEHSFAGMPC*GIMDQFISLMG | 200 |
| C182 | | QK.G | |
| Q06330_ | RBPJ Recombining binding protein | R.IIQFQATPC*PK.E | 201 |
| C313 | suppressor of hairless | | |
| Q9NVC6_ | MED17 Mediator of RNA polymerase | K.MELLMSALSPC*LL | 202 |
| C649 | II transcription subunit | | |
| Q99832_ | CCT7 T-complex protein 1 subunit eta | R.INALTAASEAAC*LIVSVDETIK.N | 203 |
| C511 | | | |
| Q9BTE3_ | MCMBP Mini-chromosome | K.LQHINPLLPAC*LNKEESK.T | 204 |
| C325 | maintenance complex-binding protein | | |
| P04183_ | TK1 Thymidine kinase, cytosolic | R.KLFAPQQILQC*SPAN | 205 |
| C230 | | | |
| Q96S55_ | WRNIP1 ATPase WRNIP1 | R.SLLETNEIPSLILWGPPGC*GK.T | 206 |
| C272 | | | |
| Q0VGL1_ | C7orf59 UPF0539 protein C7orf59 | R.IPDQLGYLVLSEGAVLASSGDLENDEQA | 207 |
| C51 | | ASAISELVSTAC*GFR.L | |
| Q86W42_ | THOC6 THO complex subunit 6 | R.LHMTIFSQSVSPC*GK.F | 208 |
| C35 | homolog | | |
| O14980_ | XPO1 Exportin-1 | K.DLLGLC*EQKR.G | 209 |
| C528 | | | |
| P43246_ | MSH2 DNA mismatch repair protein | K.HVIEC*AK.Q | 210 |
| C843 | Msh2 | | |
| Q00839_ | HNRNPU Heterogeneous nuclear | R.APQC*LGK.F | 211 |
| C562 | ribonucleoprotein U | | |
| Q8WW01 | TSEN15 tRNA-splicing endonuclease | R.GDSEPTPGC*SGLGPGGVR.G | 212 |
| C13 | subunit Sen15 | | |
| O60610_ | DIAPH1 Protein diaphanous homolog | R.KAGC*AVTSLLASELTK.D | 213 |
| C1227 | 1 | | |
| Q9NVM9_ | Asun Protein asunder homolog | R.ISPVDVNSRPSSC*LTNFLLNGR.S | 214 |
| C349 | | | |
| O15067_ | PFAS | K.FC*DNSSAIQGK.E | 215 |
| C270 | Phosphodbosylformylglycinamidine | | |
| | synthase | | |
| P38606_ | ATP6V1A V-type proton ATPase | R.VLDALFPCVQGGTTAIPGAFGC*GK.T | 216 |
| C254 | catalytic subunit A | | |
| Q8NBS9_ | TXNDC5 Thioredoxin domain- | K.FFAPWC*GHCK.A | 217 |
| C217 | containing protein 5 | | |
| P35611_ | ADD1 Alpha-adducin | R.VSMILQSPAFC*EELESMIQEQFKK.G | 218 |
| C68 | | | |
| P42224_ | STAT1 Signal transducer and | R.NLSFFLTPPC*AR.W | 219 |
| C492 | activator of transcription 1 | | |
| P46940_ | IQGAP1 Ras GTPase-activating-like | K.KQIPAITC*IQSQWR.G | 220 |
| C781 | protein IQGAP1 | | |
| O95229_ | ZWINT ZW10 interactor | K.DKLLC*SQLQVADFLQNILAQEDTAK.G | 221 |
| C54 | | | |
| Q969Q0_ | RPL36AL 60S ribosomal protein | R.C*KHFELGGDK.K | 222 |
| C88 | L36a-like | | |
| Q29RF7_ | PDS5A Sister chromatid cohesion | R.TVQTIEAC*IANFFNQVLVLGR.S | 223 |
| C242 | protein PDS5 homolog A | | |
| Q2NL82_ | TSR1 Pre-rRNA-processing protein | R.DTGTVHLNELGNTQNFMLLC*PR.L | 224 |
| C126 | TSR1 homolog | | |
| Q9UKX7_ | NUP50 Nuclear pore complex protein | K.AC*VGNAYHK.Q | 225 |
| C151 | Nup50 | | |
| P18621_ | RPL17 60S ribosomal protein L17 | R.INPYMSSPC*HIEMILTEK.E | 226 |
| C144 | | | |
| P49189_ | ALDH9A1 4- | K.GALMANFLTQGQVC*CNGTR.V | 227 |
| C288 | trimethylaminobutyraldehyde | | |
| | dehydrogenase | | |
| P45985_ | MAP2K4 Dual specificity mitogen- | K.LC*DFGISGQLVDSIAK.T | 228 |
| C246 | activated protein kinase | | |
| Q13428_ | TCOF1 Treacle protein | K.KGAGNPQASTLALQSNITQC*LLGQPWPL | 229 |
| C1298 | | NEAQVQASVVK.V | |
| Q9BRF8_ | CPPED1 Calcineurin-like | K.AWSTGDC*DNGGDEWEQEIR.L | 230 |
| C54 | phosphoesterase domain-containing | | |
| O00170_ | AIP AH receptor-interacting protein | R.HCC*GVAQMR.E | 231 |
| C122 | | | |
| Q9UNI6_ | DUSP12 Dual specificity protein | R.QAQC*TSYFIEPVQWMESALLGVMDGQL | 232 |
| C265 | phosphatase 12 | LCPK.C | |
| P52657_ | GTF2A2 Transcription initiation | R.FC*DNVWTFVLNDVEFR.E | 233 |
| C68 | factor IIA subunit 2 | | |
| O15067_ | PFAS | R.GLAPLHWADDDGNPTEQYPLNPNGSPGG | 234 |
| C1287 | Phosphoribosylformylglycinamidine | VAGICSC*DGR.H | |
| | synthase | | |
| P37235_ | HPCAL1 Hippocalcin-like protein 1 | R.LLQC*DPSSASQF | 235 |
| C185 | | | |
| P53602_ | MVD Diphosphomevalonate | R.DGDPLPSSLSC*K.V | 236 |
| C108 | decarboxylase | | |
| Q96C19_ | EFHD2 EF-hand domain-containing | K.AAAGELQEDSGLC*VLAR.L | 237 |
| C172 | protein D2 | | |
| Q9NQC3_ | RTN4 Reticulon-4 | K.YSNSALGHVNC*TIK.E | 238 |
| C1101 | | | |
| O43175_ | PHGDH D-3-phosphoglycerate | K.NAGNC*LSPAVIVGLLK.E | 239 |
| C369 | dehydrogenase | | |
| Q99638_ | RAD9A Cell cycle checkpoint control | R.LVVQLHC*K.F | 240 |
| C114 | protein RAD9A | | |
| Q06830_ | PRDX1 Peroxiredoxin-1 | K.LNCQVIGASVDSHFC*HLAWVNTPK.K | 241 |
| C83 | | | |
| Q00796_ | SORD Sorbitol dehydrogenase | R.MHSVGIC*GSDVHYWEYGR.I | 242 |
| C45 | | | |
| Q9NRG0_ | CHRAC1 Chromatin accessibility | K.ATELFVQC*LATYSYR.H | 243 |
| C55 | complex protein 1 | | |
| P31949_ | S100A11 Protein S100-A11 | K.KLDTNSDGQLDFSEFLNLIGGLAMAC*H | 244 |
| C91 | | DSFLK.A | |
| Q9NY65_ | TUBA8 Tubulin alpha-8 chain | R.TIQFVDWC*PTGFK.V | 245 |
| C347 | | | |
| Q9NXV6_ | CDKN2AIP CDKN2A-interacting | K.SVYLGTGC*GK.S | 246 |
| C516 | protein | | |
| Q13162_ | PRDX4 Peroxiredoxin-4 | R.TREEEC*HFYAGGQVYPGEASR.V | 247 |
| C51 | | | |
| Q14997_ | PSME4 Proteasome activator complex | K.QQFTDDQLLVLTDLLVSPC*YYA | 248 |
| C1840 | subunit 4 | | |
| Q15003_ | NCAPH Condensin complex subunit 2 | R.TMC*PLLSMXPGEYSYFSPR.T | 249 |
| C418 | | | |
| Q01433_ | AMPD2 AMP deaminase 2 | R.SLPGPAPC*LK.H | 250 |
| C107 | | | |
| O75874_ | IDH1 Isocitrate dehydrogenase | K.SEGGFIWAC*K.N | 251 |
| C269 | | | |
| Q29RF7_ | PDS5A Sister chromatid cohesion | R.ELLDLHKQPTSEANC*SAMFGK.L | 252 |
| C532 | protein PDS5 homolog A | | |
| P11498_ | PC Pyruvate carboxylase, | FLYECPWRR.FLYEC*PWR.R | 253 |
| C622 | mitochondrial | | |
| P49411_ | TUFM Elongation factor Tu, | K.NMITGTAPLDGC*ILVVAANDGPMPQTR. | 254 |
| C147 | mitochondrial | E | |
| Q9H0C8_ | ILKAP Integrin-linked kinase- | R.C*GVTSVPDIR.R | 255 |
| C301 | associated serine/threonine | | |
| Q02252_ | ALDH6A1 Methyhrialonate- | R.C*MALSTAVLVGEAK.K | 256 |
| C317 | semialdehyde dehydrogenase | | |
| O00487_ | PSMD14 26S proteasome non- | K.SWMEGLTLQDYSEHC*K.H | 257 |
| C238 | ATPase regulatory subunit 14 | | |
| P23919_ | DTYMK Thymidylate kinase | K.ENFSLDWC*K.Q | 258 |
| C117 | | | |
| O75362_ | ZNF217 Zinc finger protein 217 | R.C*IPQLDPFTTFQAWQLATK.G | 259 |
| C286 | | | |
| P10644_ | PRKAR1A cAMP-dependent protein | R.EC*ELYVQK.H | 260 |
| C18 | kinase type I-alpha regulatory subunit | | |
| | alpha | | |
| P51610_ | HCFC1 Host cell factor 1 | R.VAGINAC*GR.G | 261 |
| C1872 | | | |
| P0CB43_ | FAM203B Protein FAM203B | R.HVLALTGC*GPGR.A | 262 |
| C51 | | | |
| O60825_ | PFKFB2 6-phosphofructo-2- | K.VFFVESVC*DDPDVIAANILEVK.V | 263 |
| C158 | kinase/fructose-2,6-bisphosphatase | | |
| Q15813_ | TBCE Tubulin-specific chaperone E | R.NCAVSC*AGEK.G | 264 |
| C141 | | | |
| P30044_ | PRDX5 Peroxiredoxin-5, | K.GVLFGVPGAFTPGC*SK.T | 265 |
| C100 | mitochondrial | | |
| Q8N1F7_ | NUP93 Nuclear pore complex protein | K.SSGQSAQLLSHEPGDPPC*LR.R | 266 |
| C522 | Nup93 | | |
| Q9P0J1_ | PDP1 | R.GMLLGVFDGHAGC*ACSQAVSER.L | 267 |
| C149 | | | |
| P07858_ | CTSB Cathepsin B | R.GQDHC*GIESEVVAGEPR.T | 268 |
| C319 | | | |
| P12814_ | ACTN1 Alpha-actinin-1 | R.MVSDINNAWGC*LEQVEK.G | 269 |
| C370 | | | |
| O15294_ | OGT UDP-N-acetylglucosamine- | K.VMAEANHFIDLSQIPC*NGK.A | 270 |
| C620 | peptide N-acetylglucosaminyl | | |
| | transferase | | |
| Q86WS5_ | TMPRSS12 Transmembrane protease | R.RREGGAHAEDC*GTAPLK.D | 271 |
| C64 | serine 12 | | |
| Q9NQW6_ | ANLN Actin-binding protein anillin | K.NNAFPC*QVNIK.Q | 272 |
| C712 | | | |
| Q9NY27_ | PPP4R2 Serine/threonine-protein | K.EVC*PVLDQFLCHVAIGT | 273 |
| C22 | phosphatase 4 regulatory | | |
| P40763_ | STAT3 Signal transducer and | R.QQIACIGGPPNIC*LDRLENWITSLAESQL | 274 |
| C259 | activator of transcription 3 | QTR.Q | |
| O60701_ | UGDH UDP-glucose 6- | K.ASVGFGGSC*FQKDVLNLVYLCEALNLP | 275 |
| C276 | dehydrogenase | EVAR.Y | |
| Q09666_ | AHNAK Neuroblast differentiation- | R.EVFSSC*SSEVVLSGDDEEYQR.I | 276 |
| C108 | associated protein AHNA | | |
| P49773_ | HINT1 Histidine triad nucleotide- | K.C*AADLGLNK.G | 277 |
| C84 | binding protein 1 | | |
| O95071_ | UBR5 E3 ubiquitin-protein ligase | R.SFYTAIAQAFLSNEKLPNLEC*IQNANK.G | 278 |
| C2314 | UBR5 | | |
| Q9Y3Z3_ | SAMHD1 SAM domain and HD | K.NPIDHVSFYC*K.T | 279 |
| C522 | domain-containing protein 1 | | |
| O75717_ | WDHD1 WD repeat and HMG-box | K.MLALSC*K.L | 280 |
| C773 | DNA-binding protein 1 | | |
| Q9NPF4_ | OSGEP Probable tRNA | R.AMAHCGSQEALIVGGVGC*NVR.L | 281 |
| C265 | threonylcarbamoyladenosine | | |
| | biosynthesis protein OSGEP | | |
| P16455_ | MGMT Methylated-DNA-protein- | R.VVC*SSGAVGNYSGGLAVK.E | 282 |
| C150 | cysteine methyltransferase | | |
| P49327_ | FASN Fatty acid synthase | R.HAQPTC*PGAQLCTVYYASLNFR.D | 283 |
| C1558 | | | |
| Q96GG9_ | DCUN1D1 DCN1-like protein 1 | R.AATQC*EFSK.Q | 284 |
| C115 | | | |
| P13797_ | PL,S3 Plastin-3 | K.DELDELKEAFAKVDLNSNGFIC*DYELHE | 285 |
| C33 | | LFK.E | |
| P37198_ | NUP62 Nuclear pore glycoprotein p62 | K.DIIEHLNTSGAPADTSDPLQQIC*K.I | 286 |
| C475 | | | |
| P00813_ | ADA Adenosine deaminase | K.FDYYMPAIAGC*R.E | 287 |
| C75 | | | |
| O75153_ | KIAA0664 Clustered mitochondria | R.IATPFQVYSWTAPQAEHAMDC*VR.A | 288 |
| C333 | protein homolog | | |
| A6NDU8_ | C5orf51 UPF0600 protein C5orf51 | R.C*PIQLNEGVSFQDLDTAK.L | 289 |
| C179 | | | |
| Q9BWU1_ | CDK19 Cyclin-dependent kinase 19 | R.ITSEQALQDPYFQEDPLPTLDVFAGC*QIP | 290 |
| C349 | | YPK.R | |
| Q15208_ | STK38 Serine/threonine-protein | K.LSDFGLC*TGLK.K | 291 |
| C234 | kinase 38 | | |
| Q9UKV3_ | ACIN1 Apoptotic chromatin | K.AAPC*IYWLPLTDSQIVQK.E | 292 |
| C1223 | condensation inducer in the nucleus | | |
| P45984_ | MAPK9 Mitogen-activated protein | R.TAC*TNFMMTPYVVTR.Y | 293 |
| C177 | kinase 9 | | |
| Q14204_ | DYNC1H1 Cytoplasmic dynein 1 | K.TSAPITC*ELLNK.Q | 294 |
| C1999 | heavy chain 1 | | |
| P29590_ | PML Protein PML | R.LQDLSSC*ITQGK.D | 295 |
| C389 | | | |
| P62333_ | PSMC6 26S protease regulatory | R.DHQPC*IIFMDEIDAIGGR.R | 296 |
| C228 | subunit 10B | | |
| P13796_ | LCP1 Plastin-2 | K.EGIC*AIGGTSEQSSVGTQHSYSEEEK.Y | 297 |
| C101 | | | |
| Q9NWV8_ | BABAM1 BRISC and BRCA1-A | R.TILVYSRPPC*QPQFSLTEPMKK.M | 298 |
| C222 | complex member 1 | | |
| O14879_ | IFIT3 Interferon-induced protein with | K.GLNPLNAYSDLAEFLETEC*YQTPFNK.E | 299 |
| C343 | tetratricopeptide | | |
| P50749_ | RASSF2 Ras association domain- | R.ILQGPC*EQISK.V | 300 |
| C251 | containing protein 2 | | |
| O43396_ | TXNL1 Thioredoxin-like protein 1 | R.GC*GPCLR.I | 301 |
| C34 | | | |
| P35658_ | NUP214 Nuclear pore complex | K.AC*FQVGTSEEMK.M | 302 |
| C728 | protein Nup214 | | |
| P42166_ | TMPO Lamina-associated polypeptide | K.GGTLFGGEVC*K.V | 303 |
| C684 | 2, isoform alpha | | |
| Q96FX7_ | TRMT61A tRNA (adenine(58)-N(1))- | R.FCSFSPC*IEQVQR.T | 304 |
| C209 | methyltransferase catalytic | | |
| Q71U36_ | TUBA1A Tubulin alpha-1A chain | K.LEFSIYPAPQVSTAVVEPYNSILTTHTTLE | 305 |
| C200 | | HSDC*AFMVDNEAIYDICR.R | |
| O75822_ | EIF3J Eukaryotic translation initiation | K.ITNSLTVLC*SEK.Q | 306 |
| C207 | factor 3 subunit | | |
| Q9UBQ7_ | GRHPR Glyoxylate | R.QPRPEEAAEFQAEFVSTPELAAQSDFTVV | 307 |
| C216 | reductase/hydroxypyruvate reductase | AC*SLTPATEGLCNK.D | |
| P51946_ | CCNH Cyclin-H | K.ENRTC*LSQLLDIMK.S | 308 |
| C244 | | | |
| P05091_ | ALDH2 Aldehyde dehydrogenase, | K.SPNIIMSDADMDWAVEQAHFALFFNQGQ | 309 |
| C319 | mitochondrial | CC*CAGSR.T | |
| Q9NZL4_ | HSPBP1 Hsp70-binding protein 1 | R.LLDRDAC*DTVR.V | 310 |
| C204 | | | |
| O75439_ | PMPCB Mitochondrial-processing | K.FIEFGDSLC*THK.G | 311 |
| C265 | peptidase subunit beta | | |
| Q16527_ | CSRP2 Cysteine and glycine-rich | R.C*CFLCMVCR.K | 312 |
| C33 | protein 2 | | |
| Q8TC07_ | TBC1D15 TBC1 domain family | R.TLLVNC*QNK.S | 313 |
| C197 | member 15 | | |
| P07858_ | CTSB Cathepsin B | K.IC*EPGYSPTYK.Q | 314 |
| C211 | | | |
| Q15149_ | PLEC Plectin | K.GRLC*FEGLR.S | 315 |
| C3110 | | | |
| Q9NUP9_ | LIN7C Protein lin-7 homolog C | R.VLQSEFC*NAVR.E | 316 |
| C47 | | | |
| Q14683_ | SMC1A Structural maintenance of | R.EALIEIDYGDLC*EDLKDAQAEEEIKQEM | 317 |
| C987 | chromosomes protein lA | NTLQQK.L | |
| P09110_ | ACAA1 3-ketoacyl-CoA thiolase, | K.ARDC*LIPMGITSENVAER.F | 318 |
| C177 | peroxisomal | | |
| A9UHW6_ | MEF4GD MIF4G domain-containing | K.VANVIVDHSLQDC*VFSK.E | 319 |
| C49 | protein | | |
| Q9NPH0_ | ACP6-Lysophosphatidic acid | R.MGIDSSDKVDFFILLDNVAAEQAHNLPSC | 320 |
| C267 | phosphatase type 6 | *PMLK.R | |
| Q92878_ | RAD50 DNA repair protein RAD50 | RAD50 K.C*SVSSLGFNVH | 321 |
| C1302 | | | |
| O14933_ | UBE2L6 Ubiquitin/ISG15- | K.IYHPNVDENGQICLPIISSENWKPC*TK.T | 322 |
| C98 | conjugating enzyme E2 L6 | | |
| P39023_ | RPL3 60S ribosomal protein L3 | K.VAC*IGAWHPAR.V | 323 |
| C253 | | | |
| Q9UHD8_ | SEPT9 Septin-9 | K.WGTIEVENTTHC*EFAYLR.D | 324 |
| C531 | | | |
| P42167_ | TMPO Lamina-associated polypeptide | K.EMFPYEASTPTGISASC*R.R | 325 |
| C363 | 2, isoforms beta/gamma | | |
| Q14790_ | CASP8 Caspase-8 | K.VFFIQAC*QGDNYQK.G | 326 |
| C360 | | | |
| Q15149_ | PLEC Plectin | K.YLTC*PK.T | 327 |
| C4574 | | | |
| Q9UJY4_ | GGA2 ADP-ribosylation factor- | R.NLLDLLSAQPAPC*PLNYVSQK.S | 328 |
| C429 | binding protein GGA2 | | |
| Q15306_ | IRF4 Interferon regulatory factor 4 | R.DYVPDQPHPEIPYQC*PMTFGPR.G | 329 |
| C194 | | | |
| Q6L8Q7_ | PDE12 2,5-phosphodiesterase 12 | K.SRPNASGGAAC*SGPGPEPAVFCEPVVK. | 330 |
| C108 | | L | |
| Q8TDP1_ | RNASEH2C Ribonuclease H2 subunit | R.DAVPATLHLLPC*EVAVDGPAPVGR.F | 331 |
| C34 | C | | |
| Q13045_ | FLU Protein flightless-1 homolog | R.TGLC*YLPEELAALQK.L | 332 |
| C46 | | | |
| O95881_ | TXNDC12 Thioredoxin domain- | K.SWC*GACK.A | 333 |
| C66 | containing protein 12 | | |
| Q9Y296_ | TRAPPC4 Trafficking protein particle | R.C*ELFDQNLK.L | 334 |
| C195 | complex subunit 4 | | |
| Q00765_ | REEP5 Receptor expression- | K.NC*MTDLLAK.L | 335 |
| C18 | enhancing protein 5 | | |
| Q86X76_ | NIT1 Nitrilase homolog 1 | K.THLC*DVEIPGQGPMCESNSTMPGPSLES | 336 |
| C165 | | PVSTPAGK.I | |
| P52597_ | HNRNPF Heterogeneous nuclear | R.GLPFGC*TK.E | 337 |
| C122 | ribonucleoprotein F | | |
| Q96HE7_ | ERO1L ERO1-like protein alpha | R.CFC*QVSGYLDDCTCDVETIDR.F | 338 |
| C37 | | | |
| Q9BUH6_ | C9orf142 Uncharacterized protein | R.C*PGESLINPGFK.S | 339 |
| C180 | C9orf142 | | |
| Q13158_ | FADD Protein FADD | R.RVDDFEAGAAAGAAPGEEDLC*AAFNVI | 340 |
| C98 | | CDNVGK.D | |
| Q9UBE0_ | SAE1 SUMO-activating enzyme | K.KVVFC*PVK.E | 341 |
| C214 | subunit 1 | | |
| Q9P2J5_ | LARS Leucine--tRNA ligase, | K.NLETFC*EETR.R | 342 |
| C554 | cytoplasmic | | |
| Q96PU8_ | QK1 Protein quaking | K.LMSSLPNFC*GIFNHLER.L | 343 |
| C35 | | | |
| Q6XZF7_ | DNMBP Dynamin-binding protein | R.SLDQTSPC*PLVLVR.I | 344 |
| C691 | | | |
| Q9Y4R8_ | TELO2 Telomere length regulation | R.MDILDVLTLAAQELSRPGC*LGR.T | 345 |
| C628 | protein TEL2 homolog | | |
| Q9HBM1_ | SPC25 Kinetochore protein Spc25 | K.STDTSC*QMAGLR.D | 346 |
| C27 | | | |
| P21980_ | TGM2 Protein-glutamine gamma- | K.YGQC*WVFAAVACTVLR.C | 347 |
| C277 | glutamyltransferase 2 | | |
| Q9H3U1_ | UNC45A Protein unc-45 homolog A | RAIQTVSCLLQGPC*DAGNR.A | 348 |
| C426 | | | |
| Q9NRW3_ | APOBEC3C Probable DNA dC- dU- | R.LYYFQYPC*YQEGLR.S | 349 |
| C130 | editing enzyme APOBEC-3C | | |
| P55265_ | ADAR Double-stranded RNA-specific | K.NFYLC*PV | 350 |
| C1224 | adenosine deaminase | | |
| P48735_ | IDH2 Isocitrate dehydrogenase | K.SSGGFVWAC*K.N | 351 |
| C308 | | | |
| O60664_ | PLIN3 Perilipin-3 | R.VASMPLISSTC*DMVSAAYASTK.E | 352 |
| C39 | | | |
| Q9NYL9_ | TMOD3 Tropomodulin-3 | K.FC*NIMGSSNGVDQEHFSNVVK.G | 353 |
| C150 | | | |
| Q9HCC0_ | MCCC2 Methylcrotonoyl-CoA | K.NIAQIAVVMGSC*TAGGAYVPAMADENII | 354 |
| C216 | carboxylase beta chain, mitochondrial | VR.K | |
| Q16831_ | UPP1 Uridine phosphorylase 1 | R.IGTSGGIGLEYGTVVITEQAVDTC*FK.A | 355 |
| C162 | | | |
| O00244_ | ATOX1 Copper transport protein | K.HEFSVDMTC*GGCAEAVSR.V | 356 |
| C12 | ATOX1 | | |
| P11216_ | PYGB Glycogen phosphorylase, brain | R.TC*FEIFPDK.V | 357 |
| C326 | form | | |
| P42166_ | TMPO Lamina-associated polypeptide | R.NLFISC*K.S | 358 |
| C280 | 2, isoform alpha | | |
| O15372_ | EIF3H Eukaryotic translation | R.MDSLLIAGQINTYC*QNIK.E | 359 |
| C327 | initiation factor 3 subunit | | |
| Q9BRP1_ | PDCD2L Programmed cell death | R.LLHVFACAC*PGCSTGGAR.S | 360 |
| C82 | protein 2-like | | |
| Q15021_ | NCAPD2 Condensin complex subunit | K.VACC*PLER.C | 361 |
| C767 | 1 | | |
| P30042_ | C21orf33 ES1 protein homolog, | K.EFHQAGKPIGLCC*IAPVLAAK.V | 362 |
| C177 | mitochondrial | | |
| Q13425_ | SNTB2 Beta-2-syntrophin | R.LVHSGSGC*R.S | 363 |
| C391 | | | |
| O00159_ | MYO1C Unconventional myosin-1c | R.C*PENAFFLDHVR.T | 364 |
| C802 | | | |
| O95071_ | UBR5 E3 ubiquitin-protein ligase | R.C*ATTPMAVHR.V | 365 |
| C2267 | UBR5 | | |
| Q02809_ | PLOD1 Procollagen-lysine,2- | R.VGVDYEGGGC*R.F | 366 |
| C680 | oxoglutarate 5-dioxygenase 1 | | |
| Q96RE7_ | NACC1 Nucleus accumbens- | R.NTLANSC*GTGHLS | 367 |
| C416 | associated protein 1 | | |
| Q9UPQ0_ | LIMCH1 LIM and calponin homology | K.AANSC*TSYSGTTLNLKEFEGLLAQMR.K | 368 |
| C140 | domains-containing protein 1 | | |
| O14776_ | TCERG1 Transcription elongation | R.YLVLDC*VPEER.R | 369 |
| C1062 | regulator 1 | | |
| Q9H410_ | DSN1 Kinetochore-associated protein | K.VFDC*MELVMDELQGSVK.Q | 370 |
| C287 | DSNI homolog | | |
| Q12959_ | DLG1 Disks large homolog 1 | K.LLAVNNVC*LEEVTHEEAVTALK.N | 371 |
| C378 | | | |
| Q86UV5_ | USP48 Ubiquitin carboxyl-terminal | R.IWLEPC*IR.G | 372 |
| C39 | hydrolase 48 | | |
| P50454_ | SERPINH1 Serpin H1 | K.QHYNC*EHSK.I | 373 |
| C156 | | | |
| Q99798_ | ACO2 Aconitate hydratase, | R.DLGGIVLANACGPC*IGQWDR.K | 374 |
| C451 | mitochondrial | | |
| Q9P2T1_ | GMPR2 GMP reductase 2 | K.VGIGPGSVC*TTR.K | 375 |
| C186 | | | |
| Q9BTW9 | TBCD Tubulin-specific chaperone D | K.AGAPDEAVCGENVSQIYC*ALLGCMODY | 376 |
| C850 | | TTDSR.G | |
| Q8NFH5_ | NUP35 Nucleoporin NUP53 | R.C*ALSSPSLAFTPPIK.T | 377 |
| C255 | | | |
| P32322_ | PYCR1 Pyrroline-5-carboxylate | R.C*MTNTPVVVR.E | 378 |
| C120 | reductase 1, mitochondrial | | |
| P50851_ | LRBA Lipopolysaccharide-responsive | R.SLVNIPADGVTVDPALLPPAC*LGALGDL | 379 |
| C1704 | and beige-like anchor protein | SVEQPVQFR.S | |
| Q9UBR2_ | CTSZ Cathepsin Z | K.DQECDKFNQCGTC*NEFK.E | 380 |
| C173 | | | |
| Q8IWV8_ | UBR2 E3 ubiquitin-protein ligase | R.GNPLHLC*K.E | 381 |
| C1717 | UBR2 | | |
| O75369_ | FLNB Filamin-B | KIEC*SDNGDGTCSVSYLPTKPGEYFVNILF | 382 |
| C1087 | | EEVHIPGSPFK.A | |
| Q9Y4P1_ | ATG4B Cysteine protease ATG4B | K.NFPAIGGTGPTSDTGWGC*MLR.C | 383 |
| C74 | | | |
| Q8TD19_ | NEK9 Serine/threonine-protein kinase | R.LLTFGC*NK.C | 384 |
| C623 | Nek9 | | |
| Q9UMSO_ | NFU1 NFU1 iron-sulfur cluster | K.LQGSCTSC*PSSITILK.N | 385 |
| C213 | scaffold homolog, mitochondrial | | |
| O94953_ | KDM4B Lysine-specific demethylase | R.TEPYCAICTLFYPYC*QALQTEK.E | 386 |
| C694 | 4B | | |
| QI5149_ | PLEC Plectin | K.VLSSSGSEAAVPSVC*FLVPPPNQEAQEA | 387 |
| C992 | | VTR.L | |
| P14735_ | IDE Insulin-degrading enzyme | R.EMDSCPVVGEFPC*QNDINLSQAPALPQP | 388 |
| C974 | | EVIQNMTEFKR.G | |
| Q9NVG8_ | TBC1D13 TBC1 domain family | K.SLDDSQC*GITYK.M | 389 |
| C282 | member 13 | | |
| Q86TI2_ | DPP9 Dipeptidyl peptidase 9 | R.C*PESGEHYEVTLLHFLQEYL | 390 |
| C844 | | | |
| O94992_ | HEXIM1 Protein HEXIM1 | R.AFPQLGGRPGPEGEGSLESQPPPLQTQAC | 391 |
| C84 | | PESSC*LR.E | |
| Q86YH6_ | PDSS2 Decaprenyl-diphosphate | R.C*LLSDELSNIAMQVRX | 392 |
| C71 | synthase subunit 2 | | |
| P19447_ | ERCC3 TFIIH basal transcription | R.SGVIVLPC*GAGK.S | 393 |
| C342 | factor complex helicase | | |
| Q9Y3D2_ | MSRB2 Methionine-R-sulfoxide | K.YC*SGTGWPSFSEAHGTSGSDESHTGILR. | 394 |
| C105 | reductase B2, mitochondrial | R | |
| Q9P253_ | VPS18 Vacuolar protein sorting- | R.SAVLQPGC*PSVGIPHSGYVNAQLEK.E | 395 |
| C22 | associated protein 18 homolog | | |
| O75150_ | RNF40 E3 ubiquitin-protein ligase | R.EIQPC*LAESR.A | 396 |
| C890 | BREIB | | |
| P04818_ | TYMS Thymidylate synthase | R.DLPLMALPPCHALC*QFYVVNSELSCQLY | 397 |
| C199 | | QR.S | |
| Q00535_ | CDK5 Cyclin-dependent kinase 5 | R.C*YSAEVVTLWYRPPDVLFGAK.L | 398 |
| C157 | | | |
| Q96N67_ | DOCK7 Dedicator of cytokinesis | K.AVLPVTC*HR.D | 399 |
| C2125 | protein 7 | | |
| O95340_ | PAPSS2 Bifunctional 3- | R.VWGTTC*TK.H | 400 |
| C350 | phosphoadenosine 5-phosphosu1fate | | |
| Q96F86_ | EDC3 Enhancer of mRNA-decapping | R.IYLCDIGIPQQVFQEVGINYHSPFGC*K.F | 401 |
| C499 | protein 3 | | |
| Q6QNY0_ | BLOC1S3 Biogenesis of lysosome- | R.GDLC*ALAER.L | 402 |
| C168 | related organelles complex | | |
| Q7Z3B4_ | NUP54 Nucleoporin p54 | K.AVGYSC*MPSNICDEDGLVVLVFNK.K | 403 |
| C180 | | | |
| Q9UPT9_ | USP22 Ubiquitin carboxyl-terminal | R.KITSNC*TIGLR.G | 404 |
| C171 | hydrolase 22 | | |
| P45954_ | ACADSB Short/branched chain | K.VGSFC*LSEAGAGSDSFALK.T | 405 |
| C175 | specific acyl-CoA dehydrogenase | | |
| Q9UKT9_ | IKZF3 Zinc fmger protein Aiolos | R.SYELLKPPPIC*PR.D | 406 |
| C434 | | | |
| Q9NR50_ | EIF2B3 Translation initiation factor | K.EANTLNLAPYDAC*WNACR.G | 407 |
| C281 | eIF-2B subunit gamma | | |
| Q9HB90_ | RRAGC Ras-related GTP-binding | R.SC*GHQTSASSLK.A | 408 |
| C377 | protein C | | |
| Q5T0N5_ | FNBP1L Formin-binding protein 1- | R.FTSC*VAFFNILNELNDYAGQR.E | 409 |
| C69 | like | | |
| P07339_ | CTSD Cathepsin D | K.LLDIAC*WIHHK.Y | 410 |
| C117 | | | |
| Q7Z6Z7_ | HUWE1 E3 ubiquitin-protein ligase | K.AC*SPCSSQSSSSGICTDFWDLLVK.L | 411 |
| C3372 | HUWE1 | | |
| O00267_ | SUPT5H Transcription elongation | R.SFAFLHC*K.K | 412 |
| C626 | factor SPT5 | | |
| O95347_ | SMC2 Structural maintenance of | R.FTQC*QNGK.I | 413 |
| C1174 | chromosomes protein 2 | | |
| Q92616_ | GCN1L1 Translational activator | R.VLQEALC*VISGVPGLK.G | 414 |
| C648 | GCN1 | | |
| P85037_ | FOXK1 Forkhead box protein K1 | R.SMVSPVPSPTGTISVPNSC*PASPR.G | 415 |
| C254 | | | |
| P17655_ | CAPN2 Calpain-2 catalytic subunit | K.MPC*QLHQVIVAR.F | 416 |
| C640 | | | |
| P50851_ | LRBA Lipopolysaccharide-responsive | K.C*SGIGDNPGSETAAPR.A | 417 |
| C2675 | and beige-like anchor protein | | |
| O14733_ | MAP2K7 Dual specificity mitogen- | R.YQAEINDLENLGEMGSGTC*GQVWK.M | 418 |
| C131 | activated protein kinase | | |
| A6NED2_ | RCCD1 RCC1 domain-containing | R.GEPLWAQNVVPEAEGEDDPAGEAQAGR | 419 |
| C139 | protein 1 | LPLLPC*AR.A | |
| Q14980_ | NUMA1 Nuclear mitotic apparatus | R.QFC*STQAALQAMER.E | 420 |
| C961 | protein 1 | | |
| Q9NYL2_ | MLTK Mitogen-activated protein | K.FDDLQFFENC*GGGSFGSVYR.A | 421 |
| C22 | kinase kinase kinase MLT | | |
| Q13867_ | BLMH Bleomycin hydrolase | R.C*WIFSCLNVMR.L | 422 |
| C73 | | | |
| Q9BRJ7_ | NUDT16L1 Protein syndesmos | R.VLGLGLGC*LR.L | 423 |
| C88 | | | |
| Q96P48_ | ARAP1 Arf-GAP with Rho-GAP | R.AVFPEGPC*EEPLQLR.K | 424 |
| C900 | domain, ANK repeat and PH domain | | |
| | 1 | | |
| Q5MNZ6_ | WDR45L WD repeat domain | R.C*NYLALVGGGK.K | 425 |
| C63 | phosphoinositide-interacting protein 3 | | |
| Q13588_ | GRAP GRB2-related adapter protein | K.SPGAC*FAQAQFDFSAQDPSQLSFR.R | 426 |
| C161 | | | |
| H0Y2S0_ | Uncharacterized protein | R.YTQQGFGNLPIC*MAK.T | 427 |
| C31 | | | |
| Q9ULW0_ | TPX2 Targeting protein for Xklp2 | R.TVEIC*PFSFDSR.D | 428 |
| C536 | | | |
| Q9HAV4_ | XPO5 Exportin-5 | K.C*ALMEALVLISNQFK.N | 429 |
| C646 | | | |
| Q5T1V6_ | DDX59 Probable ATP-dependent | DDX59 K.NLPC*ANVR.Q | 430 |
| C414 | RNA helicase DDX59 | | |
| O14181_ | POLA2 DNA polymerase alpha | K.VLGC*PEALTGSYK.S | 431 |
| C198 | subunit B | | |
| Q8LZ73_ | RPUSD2 RNA pseudouridylate | R.LLAENEDVVVVDKPSSIPVHPC*GR.F | 432 |
| C246 | synthase domain-containing protein 2 | | |
| Q86UP2_ | KTN1 Kinectin | K.TMMFSEDEALC*VVDLLK.E | 433 |
| C303 | | | |
| Q9UGP4_ | LIMD1 LIM domain-containing | R.TPSVSAPLALSC*PR.Q | 434 |
| C305 | protein 1 | | |
| P32929_ | CTH Cystathionine gamma-lyase | K.YMNGHSDVVMGLVSVNC*ESLHNR.L | 435 |
| C229 | | | |
| Q7Z6Z7_ | HUWE1 E3 ubiquitin-protein ligase | R.HMLLLAIQEC*SEGFGLA.- | 436 |
| C4367 | HUWE1 | | |
| P31153_ | MAT2A S-adenosylmethionine | R.VHTIVISVQHDEEVC*LDEMR.D | 437 |
| C214 | synthase isoform type-2 | | |
| Q96NY7_ | CLIC6 Chloride intracellular channel | R.AGYDGESIGNC*PFSQR.L | 438 |
| C487 | protein 6 | | |
| P14635_ | CCNB1 G2/mitotic-specific cyclin-B1 | R.FMQNNC*VPK.K | 439 |
| C238 | | | |
| Q13303_ | KCNAB2 Voltage-gated potassium | R.EKVEVQLPELFHKIGVGAMTWSPLAC*GI | 440 |
| C248 | channel subunit beta-2 | VSGKYDSGIPPYSR.A | |
| Q9HAV4_ | XPO5 Exportin-5 | K.C*PICVPCGLR.L | 441 |
| C44 | | | |
| Q8ND24_ | RNF214 RING finger protein 214 | R.SSHAPATC*K.L | 442 |
| C655 | | | |
| Q9HB90_ | RRAGC Ras-related GTP-binding | R.KGLIDYNFHC*FR.K | 443 |
| C358 | protein C | | |
| Q92878_ | RAD50 DNA repair protein RAD50 | K.NIDQC*SEIVK.C | 444 |
| C1296 | | | |
| Q14149_ | MORC3 MORC family CW-type zinc | R.LSALC*PK.F | 445 |
| C15 | finger protein 3 | | |
| P13798_ | APEH Acylamino-acid-releasing | R.NPVINIASMLGSTDIPDWCVVEAGFPFSS | 446 |
| C641 | enzyme | DC*LPDLSVWAEMLDK.S | |
| Q01813_ | PFKP 6-phosphofructokinase type C | K.HEEFCVPMVMVPATVSNNVPGSDFSIGA | 447 |
| C563 | | DTALNTITDTC*DRIK.Q | |
| Q96T76_ | MMS19 MMS19 nucleotide excision | R.VGESNLTNGDEPTQC*SR.H | 448 |
| C549 | repair protein homolog | | |
| Q9BTE6_ | AARSD1 Alanyl-tRNA editing | R.VVNIEGVDSNMC*CGTHVSNLSDLQVIK.I | 449 |
| C209 | protein Aarsd1 | | |
| Q9C011_ | MTMR12 Myotubularin-related | R.HHSQQAPQAEAPC*LLR.N | 450 |
| C694 | protein 12 | | |
| P32320_ | CDA Cytidine deaminase | K.RPACTLKPEC*VQQLLVCSQEAK.K | 451 |
| C14 | | | |
| Q9P2E9_ | RRBP1 Ribosome-binding protein 1 | K.LTAEFEEAQTSAC.R.L | 452 |
| C1323 | | | |
| P30876_ | POLR2B DNA-directed RNA | R.DC*QIAHGAAQFLR.E | 453 |
| C1093 | polymerase H subunit RPB2 | | |
| P07237_ | P4HB Protein disulfide-isomerase | K.YLLVEFYAPWC*GHCK.A | 454 |
| C53 | | | |
| Q8IZ73_ | RPUSD2 RNA pseudouridylate | K.QSLDVLDLC*EGDLSPGLTDSTAPSSELG | 455 |
| C431 | synthase domain-containing protein 2 | KDDLEELAAAAQK.M | |
| Q6NZY4_ | ZCCHC8 Zinc finger CCHC domain- | R.IFGSIPMQAC*QQK.D | 456 |
| C393 | containing protein 8 | | |
| Q96EB1_ | ELP4 Elongator complex protein 4 | K.VEPC*SLTPGYTK.L | 457 |
| C218 | | | |
| O60568_ | PLOD3 Procollagen-lysine,2- | K.GLDYEGGGC*R.F | 458 |
| C691 | oxoglutarate 5-dioxygenase 3 | | |
| Q8WV74_ | NUDT8 Nucleoside diphosphate- | R.LAGLTC*SGAEGLARPK.Q | 459 |
| C207 | linked moiety X motif 8, | | |
| | mitochondrial | | |
| Q7Z6Z7_ | HUWE1 E3 ubiquitin-protein ligase | R.STDRLPSAHTC*FNQLDLPAYESFEK.L | 460 |
| C4341 | HUWE1 | | |
| P16455_ | MGMT Methylated-DNA--protein- | R.GNPVPILIPC*HR.V | 461 |
| C145 | cysteine methyltransferase | | |
| Q86X76_ | NIT1 Nitrilase homolog 1 | K.IGLAVC*YDMR.F | 462 |
| C203 | | | |
| Q9BR61_ | ACBD6 Acyl-CoA-binding domain- | R.DQDGCLPEEVTGC*K.T | 463 |
| C267 | containing protein 6 | | |
| Q8IU81_ | IRF2BP1 Interferon regulatory factor | R.EPAPAEALPQQYPEPAPAALC*GPPPRAP | 464 |
| C363 | 2-binding protein 1 | SR.N | |
| Q9Y5T5_ | USP16 Ubiquitin carboxyl-terminal | K.GLSNLGNTC*FFNAVMQNLSQTPVLR.E | 465 |
| C205 | hydrolase 16 | | |
| Q8IWZ3_ | ANKHD1 Ankyrin repeat and KH | R.AGHLC*TVQFLISK.G | 466 |
| C615 | domain-containing protein 1 | | |
| Q13158_ | FADD Protein FADD | R.RVDDFEAGAAAGAAPGEEDLCAAFNVIC | 467 |
| C105 | | *DNVGKDWRR.L | |
| P29590_ | PML Protein PML | R.GCSKPLCC*SCALLDSSHSELK.C | 468 |
| C213 | | | |
| Q00653_ | NFKB2 Nuclear factor NF-kappa-B | R.YGC*EGPSHGGLPGASSEK.G | 469 |
| C57 | p100 subunit | | |
| Q9BVA1_ | TUBB2B Tubulin beta-2B chain | K.ESESCDC*LQGFQLTHSLGGGTGSGMGT | 470 |
| C129 | | LLISK.I | |
| Q9NZA1_ | CLIC5 Chloride intracellular channel | K.AGIDGESIGNC*PFSQR.L | 471 |
| C191 | protein 5 | | |
| O60551_ | NMT2 Glycylpeptide N- | R.AMELLSAC*QGPAR.N | 472 |
| C104 | tetradecanoyltransferase 2 | | |
| Q16667_ | CDKN3 Cyclin-dependent kinase | R.VNC*SQFLGLCALPGCK.F | 473 |
| C39 | inhibitor 3 | | |
| A3KN83_ | SBNO1 Protein strawberry notch | K.NLC*PVGSSKPTK.T | 474 |
| C445 | homolog 1 | | |
| Q8N999_ | C12orf29 Uncharacterized protein | K.C*LFNHFLK.I | 475 |
| C302 | C12orf29 | | |
| Q6YN16_ | HSDL2 Hydroxysteroid | K.QHC*AYTIAK.Y | 476 |
| C166 | dehydrogenase-like protein 2 | | |
| Q9HB19_ | PLEKHA2 Pleckstrin homology | R.SISLTRPGSSSLSSGPNSILC*R.G | 477 |
| C332 | domain-containing family A member | | |
| | 2 | | |
| Q8WVV9_ | HNRPLL Heterogeneous nuclear | K.NIIQPPSCVLHYYNVPLC*VTEETFTK.L | 478 |
| C464 | ribonucleoprotein L-like | | |
| O75175_ | CNOT3 CCR4-NOT transcription | R.DIILSSTSAPPASAQPPLQLSEVNIPLSLGV | 479 |
| C600 | complex subunit 3 | C*PLGPVPLTK.E | |
| Q7L2J0_ | MEPCE 7SK snRNA | K.FQYGNYC*K.Y | 480 |
| C419 | methylphosphate capping enzyme | | |
| P36405_ | ARL3 ADP-ribosylation factor-like | R.VWQIQSCSALTGEGVQDGMNWVC*K.N | 481 |
| C174 | protein 3 | | |
| Q9Y4X0_ | AMMECR1 AMME syndrome | R.GC*IGTFSAMNLHSGLR.E | 482 |
| C175 | candidate gene 1 protein | | |
| Q9NZB2_ | FAM120A Constitutive coactivator of | K.GSQMGTVQPIPC*LLSMPTR.N | 483 |
| C531 | PPAR-gamma-like protein | | |
| P26358_ | DNMT1 DNA (cytosine-5)- | R.GVCSC*VEAGK.A | 484 |
| C1478 | methyltransferase 1 | | |
| Q8TB03_ | CXorf38 Uncharacterized protein | R.LNC*AEYK.N | 485 |
| C12 | CXorf38 | | |
| Q9Y676_ | MRPS18B 28S ribosomal protein | K.LLEQFVC*AHTGIIFYAPYTGVCVK.Q | 486 |
| C128 | S18b, mitochondrial | | |
| Q9Y613_ | FHOD1 FH1/FH2 domain-containing | R.TPQSPAPC*VLLR.A | 487 |
| C502 | protein 1 | | |
| Q99986_ | VRK1 Serine/threonine-protein kinase | K.VGLPIGQGGFGC*IYLADMNSSESVGSDA | 488 |
| C50 | VRK1 | PCVVK.V | |
| P53992_ | SEC24C Protein transport protein | R.APPSSGAPPASTAQAPC*GQAAYGQFGQ | 489 |
| C78 | Sec24C | GDVQNGPSSTVQMQR.L | |
| Q96RN5_ | MED15 Mediator of RNA polymerase | R.TFVPAMTAIHGPPITAPVVC*TR.K | 490 |
| C660 | II transcription subunit | | |
| Q27J81_ | INF2 Inverted formin-2 | R.AVLLASDAQEC*TLEEVVER.L | 491 |
| C332 | | | |
| Q27J81_ | INF2 Inverted formin-2 | K.QEEVC*VIDALLADIR.K | 492 |
| C971 | | | |
| Q9UBB4_ | ATXN10 Ataxin-10 | K.ETTNIFSNCGC*VR.A | 493 |
| C356 | | | |
| Q08378_ | GOLGA3 Golgin subfamily A | K.GEASSSNPATPIKIPDC*PVPASLLEELLRP | 494 |
| C1403 | member 3 | PPAVSKEPLK.N | |
| Q8WWI1_ | LMO7 LIM domain only protein 7 | R.DSGYGDIWC*PER.G | 495 |
| C228 | | | |
| P10398_ | ARAF Serine/threonine-protein kinase | R.TQADELPAC*LLSAAR.L | 496 |
| C597 | A-Raf | | |
| Q9NZ32_ | ACTR10 Actin-related protein 10 | R.IPDWC*SLNNPPLEMMFDVGK.T | 497 |
| C388 | | | |
| Q9Y2X3_ | NOP58 Nucleolar protein 58 | K.LNLSC*IHSPVVNELMR.G | 498 |
| C106 | | | |
| P53396_ | ACLY ATP-citrate synthase | R.LTKPIVCWCIGTCATMFSSEVQFGHAGAC | 499 |
| C764 | | *ANQASETAVAK.N | |
| P18124_ | RPL7 60S ribosomal protein L7 | K.YGIIC*MEDLIHENTVGK.R | 500 |
| C186 | | | |
| Q9BUK6_ | MST01 Protein misato homolog 1 | R.VAPPYPHLFSSC*SPPGMVLDGSPK.G | 501 |
| C485 | | | |
| O00541_ | PES1 Pescadillo homolog | K.AGEGTYALDSESC*MEK.L | 502 |
| C272 | | | |
| Q9Y277_ | VDAC3 Voltage-dependent anion- | K.VC*NYGLTFTQK.W | 503 |
| C65 | selective channel protein | | |
| Q02556_ | IRF8 Interferon regulatory factor 8 | R.VFCSGNAVVC*K.G | 504 |
| C306 | | | |
| Q01082_ | SPTBN1 Spectrin beta chain, non- | R.IHC*LENVDK.A | 505 |
| C112 | erythrocytic 1 | | |
| A2A288_ | ZC3H12D Probable ribonuclease | R.LAFSDDLGPLGPPLPVPAC*SLTPR.L | 506 |
| C367 | ZC3H12D | | |
| O14936_ | CASK Peripheral plasma membrane | R.HLEEAVELVC*TAPQWVPVSWVY | 507 |
| C914 | protein CASK | | |
| O43290_ | SART1 U4/U6.U5 tri-snRNP- | R.GLAAALLLC*QNK.G | 508 |
| C645 | associated protein 1 | | |
| Q9UBV8_ | PEF1 Peflin | K.QALVNC*NWSSFNDETCLMMINMFDK.T | 509 |
| C146 | | | |
| Q6PCB5_ | RSBN1L Round spermatid basic | K.SIQTIC*SGLLTDVEDQAAK.G | 510 |
| C280 | protein 1-like protein | | |
| Q08380_ | LGALS3BP Galectin-3-binding | K.STSSFPC*PAGHFNGFR.T | 511 |
| C561 | protein | | |
| P57737_ | CORO7 Coronin-7 | R.AGTAPSC*R.N | 512 |
| C34 | | | |
| Q92616_ | GCNIL1 Translational activator | R.C*GLALALNK.L | 513 |
| C1235 | GCN1 | | |
| P24468_ | NR2F2 COUP transcription factor 2 | K.AIVLFTSDAC*GLSDVAHVESLQEK.S | 514 |
| C326 | | | |
| Q96RP9_ | GFM1 Elongation factor G, | R.VLDGAVLVLCAVGGVQC*QTMTVNR.Q | 515 |
| C153 | mitochondrial | | |
| Q9NRF9_ | POLE3 DNA polymerase epsilon | R.AASVFVLYATSC*ANNFAMK.G | 516 |
| C51 | subunit 3 | | |
| P30837_ | ALDH1B1 Aldehyde dehydrogenase | R.HEPVGVC*GQIIPWNFPLVMQGWK.L | 517 |
| C179 | X, mitochondrial | | |
| Q8WUM4_ | PDCD6IP Programmed cell death 6- | K.FPFSENQIC*LTFTWK.D | 518 |
| C90 | interacting protein | | |
| Q9NP73_ | ALG13 UDP-N-acetylglucosamine | K.ADLVISHAGAGSC*LETLEK.G | 519 |
| C86 | transferase subunit ALG13 | | |
| P13667_ | PDIA4 Protein disulfide-isomerase A4 | K.EENGVLVLNDANFDNFVADKDTVLLEFY | 520 |
| C91 | | APWC*GHCK.Q | |
| P09884_ | POLA1 DNA polymerase alpha | R.YIFDAEC*ALEK.L | 521 |
| C1403 | catalytic subunit | | |
| O95833_ | CLIC3 Chloride intracellular channel | K.ASEDGESVGHC*PSCQR.L | 522 |
| C22 | protein 3 | | |
| Q9NTX5_ | ECHDC1 Ethylmalonyl-CoA | K.SLGTPEDGMAVC*MFMQNTLTR.F | 523 |
| C133 | decarboxylase | | |
| Q14289_ | PTK2B Protein-tyrosine kinase 2-beta | K.NELC*QLPPEGYVVVVK.N | 524 |
| C899 | | | |
| P49792_ | RANBP2 E3 SUMO-protein ligase | K.MIC*QQVEAIKKE | 525 |
| C815 | RanBP2 | | |
| Q14137_ | BOP1 Ribosome biogenesis protein | R.DLQPFPTC*QALVYR.G | 526 |
| C404 | BOP1 | | |
| Q15345_ | LRRC41 Leucine-rich repeat- | R.C*AAALMASR.R | 527 |
| C297 | containing protein 41 | | |
| Q99538_ | LGMN Legiunain | R.ESSYAC*YYDEK.R | 528 |
| C219 | | | |
| Q96EK4_ | THAP11 THAP domain-containing | R.AGVSGC*FSTFQPTTGHR.L | 529 |
| C48 | protein 11 | | |
| O60488_ | ACSL4 Long-chain-fatty-acid--CoA | K.GYDAPLC*NLLLFK.K | 530 |
| C420 | ligase 4 | | |
| Q96EY5_ | FAM125A Multivesicular body | K.SC*SPLAFSAFGDLTIK.S | 531 |
| C231 | subunit 12A | | |
| P22307_ | SCP2 Non-specific lipid-transfer | K.SGLTPNDEDVIELHDC*FSTNELLTYEALG | 532 |
| C307 | protein | LCPEGQGATLVDR.G | |
| Q969Z0_ | TBRG4 Protein TBRG4 | R.LATDLLSLMPSLTSGEVAHC*AK.S | 533 |
| C335 | | | |
| P46734_ | MAP2K3 Dual specificity mitogen- | R.ISC*MSKPPAPNPTPPR.N | 534 |
| C29 | activated protein kinase | | |
| Q5JPI3_ | C3orf38 Uncharacterized protein | K.FEQSDLEAFYNVITVC*GTNEVR.H | 535 |
| C308 | C3orf38 | | |
| Q9NRA8_ | EIF4ENIF1 Eukaryotic translation | R.DAVLPEQSPGDFDFNEFFNLDKVPC*LAS | 536 |
| C318 | initiation factor 4E transporter | MIEDVLGEGSVSASR.F | |
| Q15648_ | MED1 Mediator of RNA polymerase | K.VAHHGENPVSC*PELVQQLR.E | 537 |
| C135 | II transcription subunit | | |
| Q8IZ69_ | TRMT2A tRNA (uracil-5-)- | R.VIGVELC*PEAVEDAR.V | 538 |
| C463 | methyltransferase homolog A | | |
| Q04206_ | RELA Transcription factor p65 | R.YKC*EGR.S | 539 |
| C38 | | | |
| O15533_ | TAPBP Tapasin | K.WASGLTPAQNC*PR.A | 540 |
| C115 | | | |
| Q9UNI6_ | DUSP12 Dual specificity protein | R.VSC*AGQMLEVQPGLYEGGAAAVAEPD | 541 |
| C23 | phosphatase 12 | HLR.E | |
| Q9BUP3_ | HTATIP2 Oxidoreductase HTATIP2 | R.YSVFRPGVLLC*DR.Q | 542 |
| C172 | | | |
| P53384_ | NUBP1 Cytosolic Fe-S cluster | K.LPIIGVVENMSGFIC*PK.C | 543 |
| C235 | assembly factor NUBP1 | | |
| Q7Z5K2_ | WAPAL Wings apart-like protein | R.LENINEAIEEDIVQSVLRPTNC*R.T | 544 |
| C293 | homolog | | |
| Q9Y5S2_ | CDC42BPB Serine/threonine-protein | R.IRPLNSEGTLNLLNC*EPPR.L | 545 |
| C1517 | kinase MRCK beta | | |
| Q15024_ | EXOSC7 Exosome complex | K.GVVTC*MR.K | 546 |
| C238 | component RRP42 | | |
| P40855_ | PEX19 Permdsomal biogenesis factor | R.VGSDMTSQQEFTSC*LK.E | 547 |
| C128 | 19 | | |
| Q15910_ | EZH2 Histone-lysine N- | R.LWAAHC*R.K | 548 |
| C503 | methyltransferase EZH2 | | |
| Q15149_ | PLEC Plectin | K.FLEGTSC*IAGVFVDATK.E | 549 |
| C4071 | | | |
| P22307_ | SCP2 Non-specific lipid-transfer | R.AIYHSLGMTGIPIINVNNNC*ATGSTALFM | 550 |
| C94 | protein | AR.Q | |
| Q12789_ | GTF3C1 General transcription factor | R.VPPFPLPLEPC*TQEFLWR.A | 551 |
| C42 | 3C polypeptide 1 | | |
| Q15796_ | SMAD2 Mothers against | K.CVTIPSTC*SEIWGLSTPNTIDQWDTTGLY | 552 |
| C81 | decapentaplegic homolog 2 | SFSEQTR.S | |
| Q9UL15_ | BAGS BAG family molecular | K.TELQGLIGQLDEVSLEICNPC*IR.E | 553 |
| C327 | chaperone regulator 5 | | |
| Q8N9T8_ | KRI1 Protein KRI1 homolog | R.LLGPTVMLGGC*EFSR.Q | 554 |
| C673 | | | |
| Q9Y679_ | AUP1 Ancient ubiquitous protein 1 | K.TGC*VDLTTTNLLEGAVAFMPEDITK.G | 555 |
| C391 | | | |
| O14733_ | MAP2K7 Dual specificity mitogen- | K.LC*DFGISGR.L | 556 |
| C260 | activated protein kinase | | |
| O75179_ | ANKRD17 Ankyrin repeat domain- | R.AGHVC*TVQFLISK.G | 557 |
| C644 | containing protein 17 | | |
| P31323_ | PRKAR2B cAMP-dependent protein | R.LLGPC*MEINK.R | 558 |
| C388 | kinase type II-beta regulatory subunit | | |
| Q9HBL8_ | NMRAL1 NmrA-like family domain- | R.LPC*YFENLLSHFLPQK.A | 559 |
| C154 | containing protein 1 | | |
| Q96RU2_ | USP28 Ubiquitin carboxyl-terminal | K.NVGNTC*WFSAVIQSLFQLPEFR.R | 560 |
| C171 | hydrolase 28 | | |
| Q96T76_ | MMS19 MMS19 nucleotide excision | R.ELLELSCCHSC*PFSSTAAAK.C | 561 |
| C750 | repair protein homolog | | |
| P17858_ | PFKL 6-phosphofructokinase, liver | R.C*KAFTTR.E | 562 |
| C89 | type | | |
| Q96F07_ | CYFIP2 Cytoplasmic FMR1- | R.LCC*GLSMFEVILTR.I | 563 |
| C1112 | interacting protein 2 | | |
| P42695_ | NCAPD3 Condensin-2 complex | R.C*VMAMLR.R | 564 |
| C541 | subunit D3 | | |
| Q9Y2S7_ | POLDIP2 Polymerase delta- | R.DC*PHISQR.S | 565 |
| C143 | interacting protein 2 | | |
| P52948_ | NUP98 Nuclear pore complex protein | R.WLSC*TATPQIEEEVSLTQK.N | 566 |
| C1312 | Nup98-Nup96 | | |
| Q9UER7_ | DAXX Death domain-associated | K.IC*TLPSPPSPLASLAPVADSSTR.V | 567 |
| C664 | protein 6 | | |
| Q9H4L7_ | SMARCAD1 SWI/SNF-related | K.NTEMC*NVMMQLR.K | 568 |
| C772 | matrix-associated actin-dependent | | |
| Q9Y570_ | PPME1 Protein phosphatase | R.FAEPIGGFQC*VFPGC | 569 |
| C381 | methylesterase 1 | | |
| P52701_ | MSH6 DNA mismatch repair protein | K.TFFGDDFIPNDILIGC*EEEEQENGK.A | 570 |
| C1117 | Msh6 | | |
| P33992_ | MCM5 DNA replication licensing | R.C*SVLAAANSVFGR.W | 571 |
| C482 | factor MCM5 | | |
| E2QRD5_ | C15orf38-AP3S2 Protein C15orf38- | K.C*NFTGDGK.T | 572 |
| C183 | AP3S2 | | |
| Q16877_ | PFKFB4 6-phosphofructo-2- | K.TFFVESIC*VDPEVIAANIVQVK.L | 573 |
| C159 | kinase/fructose-2,6-bisphosphatase 4 | | |
| Q9BSQ5_ | CCM2 Malcavernin | K.VAAEELCC*LLGQVFQVVYTESTIDFLDR. | 574 |
| C211 | | A | |
| Q00013_ | MPP1 55 kDa erythrocyte membrane | K.K.DNLIPC*K.E | 575 |
| C179 | protein | | |
| Q6UWP2_ | DHRS11 Dehydrogenase/reductase | K.C*LKPEDVAEAVIYVLSTPAHIQIGDIQMR | 576 |
| C226 | SDR family member 11 | PTEQVT | |
| Q8N556_ | AFAP1 Actin filament-associated | K.SQAAPGSSPC*R.G | 577 |
| C713 | protein 1 | | |
| Q9NXJ5_ | PGPEP1 Pyroglutamyl-peptidase 1 | R.YLC*DFTYYTSLYQSHGR.S | 578 |
| C149 | | | |
| Q8IYS1_ | PM20D2 Peptidase M20 domain- | MRPGGERPVEGGAC*NGR.S | 579 |
| C14 | containing protein 2 | | |
| Q92878_ | RAD50 DNA repair protein RAD50 | K.TTIIEC*LK.Y | 580 |
| C48 | | | |
| Q68CZ2_ | TNS3 Tensin-3 | R.SC*PETLTHAVGMSESPIGPK.S | 581 |
| C888 | | | |
| O75592_ | MYCBP2 Probable E3 ubiquitin- | R.C*SILSPELALPTGSR.A | 582 |
| C1131 | protein ligase MYCBP2 | | |
| Q5VUA4_ | ZNF318 Zinc finger protein 318 | K.LSPQAC*SFTK.A | 583 |
| C1860 | | | |
| Q9H3M7_ | TXN1P Thioredoxin-interacting | K.VSC*MFIPDGR.V | 584 |
| C170 | protein | | |
| Q92619_ | HMHA1 Minor histocompatibility | R.C*EGGVDAALLYAK.N | 585 |
| C278 | protein HA-1 | | |
| P46013_ | MKI67 Antigen KI-67 | K.SEETNTEIVEC*ILK.R | 586 |
| C903 | | | |
| Q9UI30_ | TRMT112 tRNA methyltransferase | K.GPVEGYEENEEFLRTMHHLLLEVEVIEGT | 587 |
| C100 | 112 homolog | LQC*PESGR.M | |
| Q92797_ | SYMPK Symplekin | R.GMGMNSPELLLLVENC*PK.G | 588 |
| C848 | | | |
| P50990_ | CCT8 T-complex protein 1 subunit | R.NIQAC*K.E | 589 |
| C36 | theta | | |
| O76075_ | DFFB DNA fragmentation factor | R.VLGSMC*QR.L | 590 |
| C194 | subunit beta | | |
| P04150_ | NR3C1 Glucocorticoid receptor | K.LGTVYC*QASFPGANIIGNK.M | 591 |
| C302 | | | |
| Q96JH7_ | VCPIP1 Deubiquitinating protein | K.SQECLIPVHVDGDGHC*LVHAVSR.A | 592 |
| C219 | VCIP135 | | |
| Q8NEC7_ | GSTCD Glutathione S-transferase C- | K.AC*AEVSQWTR.L | 593 |
| C140 | terminal domain-containing | | |
| P48200_ | IREB2 Iron-responsive element- | K.C*AIQNAPNPGGGDLQK.A | 594 |
| C137 | binding protein 2 | | |
| Q8WTW3_ | COG1 Conserved oligomeric Golgi | K.AQAISPC*VQNFCSALDSK.L | 595 |
| C513 | complex subunit 1 | | |
| Q9BYX2_ | TBC1D2 TBC1 domain family | R.TQNC*FLNSEIHQVTK.I | 596 |
| C469 | member 2A | | |
| P51991_ | HNRNPA3 Heterogeneous nuclear | R.GFGFVTYSC*VEEVDAAMCAR.P | 597 |
| C85 | ribonucleoprotein A3 | | |
| Q5UIP0_ | RIF1 Telomere-associated protein | K.ESIPCPTESVYPPLVNC*VAPVDIILPQITS | 598 |
| C2298 | RIF1 | NMWAR.G | |
| Q5T440_ | IBA57 Putative transferase CAF17, | R.VWAVLPSSPEAC*GAASLQER.A | 599 |
| C170 | mitochondrial | | |
| P46821_ | MAP 1B Microtubule-associated | R.KSC*FWK.L | 600 |
| C293 | protein 1B | | |
| Q9BT09_ | CNPY3 Protein canopy homolog 3 | K.QC*DVLVEEFEEVIEDWYR.N | 601 |
| C166 | | | |
| Q8IXB1_ | DNAJC10 DnaJ homolog subfamily C | K.ESVNSHVTTLGPQNFPANDKEPWLVDFF | 602 |
| C480 | member 10 | APWC*PPCR.A | |
| O00622_ | CYR61 Protein CYR61 | K.C*APGVGLVR.D | 603 |
| C39 | | | |
| Q69YN2_ | CWF19L1 CWF19-like protein 1 | K.QILAPVEESAC*QFFFDLNEK.Q | 604 |
| C288 | | | |
| Q8TEX9_ | IPO4 Importin-4 | K.LLEC*PHLNVR.K | 605 |
| C708 | | | |
| Q9Y6W3_ | CAPN7 Calpain-7 | R.AHFPLGANPFLERPQSFISPQSC*DAQGQR. | 606 |
| C197 | | Y | |
| Q9Y6Y8_ | SEC23IP SEC23-interacting protein | K.C*PGPLAVANGVVK.Q | 607 |
| C604 | | | |
| Q9ULX6_ | AKAP8L A-kinase anchor protein 8- | R.GQC*MSGASR.L | 608 |
| C211 | like | | |
| Q13630_ | TSTA3 GDP-L-fucose synthase | K.VVSCLSTC*IFPDK.T | 609 |
| C116 | | | |
| P13667_ | PDIA4 Protein disulfide-isomerase A4 | K.DTVLLEFYAPWCGHC*K.Q | 610 |
| C94 | | | |
| Q5T1V6_ | DDX59 Probable ATP-dependent | K.LFKPPVLVFVDC*K.L | 611 |
| C453 | RNA helicase DDX59 | | |
| Q9GZR2_ | REXO4 RNA exonuclease 4 | K.ILGLQVQQAEHC*SIQDAQAAMR.L | 612 |
| C382 | | | |
| Q8WXD5_ | GEMIN6 Gem-associated protein 6 | K.LMHLFTSGDC*K.A | 613 |
| C91 | | | |
| Q15118_ | PDK1 | K.QFLDFGSVNAC*EK.T | 614 |
| C71 | | | |
| Q8WXE1_ | ATRIP ATR-interacting protein | RFQC*VFQVLPKC | 615 |
| C585 | | | |
| Q8N2W9_ | PIAS4 E3 SUMO-protein ligase | R.VSLIC*PLVK.M | 616 |
| C326 | PIAS4 | | |
| Q14141_ | SEPT6 Septin-6 | R.QYPWGTVQVENEAHC*DFVK.L | 617 |
| C269 | | | |
| P26358_ | DNMT1 DNA (cytosine-5)- | R.CTVEYGEDLPEC*VQVYSMGGPNR.F | 618 |
| C1071 | methyltransferase 1 | | |
| Q8IYQ7_ | THNSL1 Threonine synthase-like 1 | R.LGEMIETAYGENFAC*SK.I | 619 |
| C324 | | | |
| P49419_ | ALDH7A1 Alpha-aminoadipic | R.STC*TINYSK.D | 620 |
| C522 | semialdehyde dehydrogenase | | |
| Q9ULJ3_ | ZNF295 Zinc finger protein 295 | K.TPQAPFPTC*PNR.K | 621 |
| C129 | | | |
| Q9HB19_ | PLEKHA2 Pleckstrin homology | K.C*EQDREPLR.T | 622 |
| C232 | domain-containing family A member | | |
| | 2 | | |
| O76031_ | CLPX ATP-dependent Clp protease | K.C*ELNVTEDALK.A | 623 |
| C538 | ATP-binding subunit clp | | |
| Q01581_ | HMGCS1 Hydroxymethylglutaryl- | K.TNLMQLFEESGNTDIEGIDTTNAC*YGGT | 624 |
| C129 | CoA synthase, cytoplasmic | AAVFNAVNWIESSSWDGR.Y | |
| Q96RS6_ | NUDCD1 NudC domain-containing | R.LMHLTSEELNPNPDKEKPPC*NAQELEEC | 625 |
| C402 | protein 1 | DIFFEESSSLCR.F | |
| Q9UL45_ | PLDN Pallidin | K.FKEC*HSMLDINALFAEAK.H | 626 |
| C95 | | | |
| Q9HAU4_ | SMURF2 E3 ubiquitin-protein ligase | R.LFTIHQIDAC*TNNLPK.A | 627 |
| C706 | SMURF2 | | |
| Q8TCG1_ | KIAA1524 Protein CIP2A | K.C*LEPTVALLR.W | 628 |
| C337 | | | |
| Q9P0K7_ | RAI14 Ankycorbin | K.QILTMC*K.N | 629 |
| C973 | | | |
| Q7Z2Z2_ | EFTUD1 Elongation factor Tu GTP- | R.ICDGCIIVVDAVEGVC*PQTQAVLR.Q | 630 |
| C124 | binding domain-containing protein 1 | | |
| Q5JTD0_ | TJAP1 Tight junction-associated | R.NSPLPNC*TYATR.Q | 631 |
| C350 | protein 1 | | |
| Q15022_ | SUZ12 Polycomb protein SUZ12 | R.LQLLDGEYEVAMQEMEEC*PISK.K | 632 |
| C325 | | | |
| P30837_ | ALDH1B1 Aldehyde dehydrogenase | K.SPSIVLADADMEHAVEQCHEALFFNMGQ | 633 |
| C320 | X, mitochondrial | CCC*AGSR.T | |
| Q9H0L4_ | CSTF2T Cleavage stimulation factor | K.LC*VQNSHQEAR.N | 634 |
| C150 | subunit 2 tau variant | | |
| Q03I69_ | TNFAIP2 Tumor necrosis factor | K.GLANVFC*VFTK.G | 635 |
| C45 | alpha-induced protein 2 | | |
| Q9UBB4_ | ATXN10 Ataxin-10 | R.VIHVAGKETTNIFSNC*GCVR.A | 636 |
| C354 | | | |
| Q06187_ | BTK Tyrosine-protein kinase BTK | K.QRPIFIITEYMANGC*LLNYLR.E | 637 |
| C481 | | | |
| Q96N21_ | ENTHD2 AP-4 complex accessory | R.ALC*AIASLGSSDLLPQEHILLR.T | 638 |
| C302 | subunit tepsin | | |
| Q92878_ | RAD50 DNA repair protein RAD50 | R.SMVC*TQK.S | 639 |
| C102 | | | |
| Q7Z401_ | DENND4A C-myc promoter-binding | R.LKQGC*EIIQSTPYGRPANISGSTSSQR.I | 640 |
| C117 | protein | | |
| Q9H307_ | PNN Pinin | RMC*PATQK.L | 641 |
| C249 | | | |
| Q96DF8_ | DGCR14 Protein DGCR14 | R.C*QLQQAAALNAQHK.Q | 642 |
| C263 | | | |
| Q7L8W6_ | ATPBD4 ATP-binding domain- | K.C*EGDEVEDLYELLK.L | 643 |
| C88 | containing protein 4 | | |
| Q13232_ | NME3 Nucleoside diphosphate kinase | R.ADELLC*WEDSAGHWLYE | 644 |
| C158 | 3 | | |
| Q9NWA0_ | MED9 Mediator of RNA polymerase | K.SLC*MFEIPKE | 645 |
| C139 | II transcription subunit | | |
| Q9BZ67_ | FRMD8 FERM domain-containing | R.VQLGPYQPGRPAAC*DLR.E | 646 |
| C191 | protein 8 | | |
| P51617_ | IRAK1 Interleulcin-1 receptor- | R.SWHLTPSC*PLDPAPLR.E | 647 |
| C608 | associated kinase 1 | | |
| Q9Y4W2_ | LAS1L Ribosomal biogenesis protein | K.C*LAQEVNIPDWIVDLR.H | 648 |
| C140 | LAS1L | | |
| Q9NX18_ | SDHAF2 Succinate dehydrogenase | R.GMLENC*ILLSLFAK.E | 649 |
| C83 | assembly factor 2, mitochondrial | | |
| Q969H6_ | POP5 Ribonuclease P/MRP protein | R.SC*LLEEEEESGEEAAEAME | 650 |
| C146 | subunit POP5 | | |
| Q96IV0_ | NGLY1 Peptide-N(4)-(N-acetyl-beta- | R.C*GEWANCFTLCCR.A | 651 |
| C309 | glucosaminyl)asparagin | | |
| P42575_ | CASP2 Caspase-2 | R.SDMICGYAC*LK.G | 652 |
| C370 | | | |
| P51812_ | RPS6KA3 Ribosomal protein S6 | K.EDIGVGSYSVC*K.R | 653 |
| C436 | kinase alpha-3 | | |
| P09086_ | POU2F2 POU domain, class 2, | R.VWFC*NR.R | 654 |
| C346 | transcription factor 2 | | |
| Q8IWV7_ | UBR1 E3 ubiquitin-protein ligase | RNSLIELPDDYSC*LLNQASIEFRC | 655 |
| C1603 | UBR1 | | |
| Q9H0W8_ | SMG9 Protein SMG9 | RREDFC*PRK | 656 |
| C380 | | | |
| O96017_ | CHEK2 Serine/threonine-protein | KTLGSGAC*GEVKL | 657 |
| C231 | kinase Chk2 | | |
| Q96RU3_ | FNBP1 Formin-binding protein 1 | K.KNSKEEEEYKYTSC*K.A | 658 |
| C70 | | | |
| O95347_ | SMC2 Structural maintenance of | KKNLAC*EESKR | 659 |
| C326 | chromosomes protein 2 | | |
| O15357_ | INPPL1 Phosphatidylinositol 3,4,5- | RKPAFTEASC*PLSRL | 660 |
| C926 | trisphosphate 5-phosphatase 2 | | |
| Q8N5L8_ | RPP25L Ribonuclease P protein | R.DPLDPNEC*GYQPPGAPPGLGSMPSSSCG | 661 |
| C131 | subunit p25-like protein | PR.S | |
| Q9C0I1_ | MTMR12 Myotubularin-related | RVFQFC*LRY | 662 |
| C152 | protein 12 | | |
| Q96F86_ | EDC3 Enhancer of xnRNA-decapping | K.SQDVAVSPQQQQC*SK.S | 663 |
| C137 | protein 3 | | |
| O60443_ | DFNA5 Non-syndromic hearing | R.FWC*WQRPK.Y | 664 |
| C45 | impairment protein 5 | | |
| Q9NU22_ | MDN1 Midasin | K.LIC*QHIVPGNVK.S | 665 |
| C1011 | | | |
| Q14204_ | DYNC1H1 Cytoplasmic dynein 1 | K.VWLQYQC*LWDMQAENIYNR.L | 666 |
| C1059 | heavy chain 1 | | |
| Q09472_ | EP300 Histone acetyltransferase p300 | R.C*IQSLVHACQCR.N | 667 |
| C1738 | | | |
| P12532_ | CKMT1B Creatine kinase U-type, | R.LGYILTC*PSNLGTGLR.A | 668 |
| C316 | mitochondrial | | |
| Q9P2X3_ | IMPACT Protein IMPACT | R.STFQAHLAPVVC*PK.Q | 669 |
| C195 | | | |
| Q92667_ | AKAP1 A-kinase anchor protein 1, | K.SIPLEC*PLSSPK.G | 670 |
| C147 | mitochondrial | | |
| O14879_ | IFIT3 Interferon-induced protein with | R.VLESTPNNGYLYHQIGC*CYK.A | 671 |
| C283 | tetratricopeptide | | |
| Q01201_ | RELB Transcription factor Relb | R.GAASLSTVTLGPVAPPATPPPWGC*PLGR. | 672 |
| C109 | | L | |
| Q9UPN9_ | TRIM33 E3 ubiquitin-protein ligase | R.GNMNC*GAFQAHQMR.L | 673 |
| C582 | TRIM33 | | |
| Q9BSD7_ | NTPCR Cancer-related nucleoside- | R.VC*VIDEIGK.M | 674 |
| C110 | triphosphatase | | |
| Q8IV53_ | DENND1C DENN domain-containing | R.GNSKPLSC*FVAPDSGR.L | 675 |
| C174 | protein 1C | | |
| Q99798_ | ACO2 Aconitate hydratase, | R.VGLIGSC*TNSSYEDMGR.S | 676 |
| C385 | mitochondrial | | |
| Q14204_ | DYNC1H1 Cytoplasmic dynein 1 | R.C*VLNWFGDWSTEALYQVGKEFTSK.M | 677 |
| C3089 | heavy chain 1 | | |
| P49903_ | SEPHS1 Selenide, water dikinase 1 | R.FTELKGTGC*K.V | 678 |
| C31 | | | |
| Q7OCQ2_ | USP34 Ubiquitin carboxyl-terminal | R.TGDFLGETIGNELFNC*R.Q | 679 |
| C741 | hydrolase 34 | | |
| O43439_ | CBFA2T2 Protein CBFA2T2 | R.FSNGPASSTSSALTNQQLPATC*GAR.Q | 680 |
| C111 | | | |
| Q9BWH6_ | RPAP1 RNA polymerase II- | R.C*GQGTLLAQACQDLPSIR.N | 681 |
| C1039 | associated protein 1 | | |
| Q6AI12_ | ANKRD40 Ankyrin repeat domain- | R.TPESTICPGPVC*QPPVSQSR.S | 682 |
| C209 | containing protein 40 | | |
| Q5GLZ8_ | HERC4 Probable E3 ubiquitin-protein | R.HTVFVLDDGTVYTCGC*NDLGQLGHEK. | 683 |
| C60 | ligase HERC4 | S | |
| Q9Y5A7_ | NUB1 NEDD8 ultimate buster 1 | R.LECC*ENEVEK.V | 684 |
| C52 | | | |
| Q6PJG6_ | BRAT1 BRCA1-associated ATM | R.TQAFQVLLQPLAC*VLK.A | 685 |
| C326 | activator 1 | | |
| Q6PJG6_ | BRAT1 BRCA1-associated ATM | R.THC*PYAVALPEVAPAQPLTEALR.A | 686 |
| C673 | activator 1 | | |
| Q8NB90_ | SPATA5 Spermatogenesis-associated | K.GVLLYGPPGC*SK.T | 687 |
| C672 | protein 5 | | |
| Q9ULT8_ | HECTD1 E3 ubiquitin-protein ligase | K.LLQLSC*NGNVK.S | 688 |
| C1855 | HECTD1 | | |
| Q9NUI1_ | DECR2 Peroxisomal 2,4-dienoyl-CoA | R.HLFC*PDLLR.D | 689 |
| C22 | reductase | | |
| Q9P0V9_ | SEPT10 Septin-10 | RQYPWGVVQVENENHC*DFVKL | 690 |
| C293 | | | |
| Q8WX93_ | PALLD Palladin | K.VSSC*EQR.L | 691 |
| C964 | | | |
| Q9Y4B4_ | RAD54L2 Helicase ARIP4 | R.AGC*LGVNLIGANR.V | 692 |
| C820 | | | |
| P41226_ | UBA7 Ubiquitin-like modifier- | R.APASAAASEDAPYPVC*TVR.Y | 693 |
| C599 | activating enzyme 7 | | |
| Q9BZQ6_ | EDEM3 ER degradation-enhancing | R.VPC*GFAAMK.D | 694 |
| C441 | alpha-mannosidase-like 3 | | |
| P52732_ | KIF11 Kinesin-like protein KIF11 | R.SVVC*PILDEVIMGYNCTIFAYGQTGTGK. | 695 |
| C87 | | T | |
| P14598_ | NCF1 Neutrophil cytosol factor 1 | R.C*SESTK.R | 696 |
| C378 | | | |
| Q9UL40_ | ZNF346 Zinc finger protein 346 | K.NQC*LFTNTQCK.V | 697 |
| C68 | | | |
| Q9C0C9_ | UBE2O Ubiquitin-conjugating | K.NC*AQGEGSMAK.K | 698 |
| C375 | enzyme E2 O | | |
| O14733_ | MAP2K7 Dual specificity mitogen- | R.SAGC*AAYMAPER.I | 699 |
| C280 | activated protein kinase | | |
| O95239_ | KIF4A Chromosome-associated | R.GLLC*LGNVISALGDDKK.G | 700 |
| C269 | kinesin KIF4A | | |
| Q8WYP5_ | AHCTF1 Protein ELYS | R.C*LVAGLLSPR.F | 701 |
| C521 | | | |
| Q93074_ | MED12 Mediator of RNA polymerase | K.TPQLNPC*QSDGNKPTVGIR.S | 702 |
| C1188 | II transcription subunit 12 | | |
| Q16610_ | ECM1 Extracellular matrix protein 1 | R.AC*PSHQPDISSGLELPFPPGVPTLDNIK.N | 703 |
| C284 | | | |
| O00622_ | CYR61 Protein CYR61 | K.HQCTCIDGAVGCIPLC*PQELSLPNLGCPN | 704 |
| C134 | | PR.L | |
| Q2VPK5_ | CTU2 Cytoplasmic tRNA 2-thiolation | R.TPPGPC*CSPGVGWAQR.C | 705 |
| C433 | protein 2 | | |
| Q9UI10_ | EIF2B4 Translation initiation factor | R.AHNVPVLVCCETYKFC*ER.V | 706 |
| C444 | eIF-2B subunit delta | | |
| Q9UPW5_ | AGTPBP1 Cytosolic | RLTSPLEYNLPSSLLDFENDLIESSC*KV | 707 |
| C1164 | carboxypeptidase 1 | | |
| Q13613_ | MTMR1 Myotubularin-related protein | KDVMYIC*PFMGAVSGTLTVTDFKL | 708 |
| C117 | I | | |
| O00541_ | PES1 Pescadillo homolog | K.SLC*IGATYDVTDSR.I | 709 |
| C361 | | | |
| P39877_ | PLA2G5 Calcium-dependent | K.VTGKNALTNYGFYGC*YCGWGGR.G | 710 |
| C46 | phospholipase A2 | | |
| Q14207_ | NPAT Protein NPAT | K.SEETTVPFPEESIVPAAKPC*HR.R | 711 |
| C1059 | | | |
| P42704_ | LRPPRC Leucine-rich PPR motif- | R.AILQENGC*LSDSDMFSQAGLR.S | 712 |
| C500 | containing protein, mitochondrial | | |
| Q9Y5R8_ | TRAPPC1 Trafficking protein particle | K.NPLC*PLGQTVQSELFR.S | 713 |
| C115 | complex subunit 1 | | |
| O60566_ | BUB1B Mitotic checkpoint | K.IPGMTLSSSVCQVNCC*AR.E | 714 |
| C504 | serine/threonine-protein kinase | | |
| Q5T440_ | IBA57 Putative transferase CAF17, | K.GC*YIGQELTAR.T | 715 |
| C259 | mitochondrial | | |
| Q5TGY3_ | AHDC1 AT-hook DNA-binding | R.GPAAAAAGYGC*PLLSDLTLSPVPR.D | 716 |
| C1540 | motif-containing protein 1 | | |
| P43403_ | ZAP70 Tyrosine-protein kinase ZAP- | R.PSGLEPQPGVFDC*LR.D | 717 |
| C117 | 70 | | |
| P07602_ | PSAP Proactivator polypeptide | KHC*LQTVWNKPTVK.S | 718 |
| C48 | | | |
| Q93074_ | MED12 Mediator of RNA polymerase | K.C*QEATAGFTIGR.V | 719 |
| C444 | II transcription subunit 12 | | |
| Q96FA3_ | PELI1 E3 ubiquitin-protein ligase | R.QEINAARPQC*PVGFNTLAFPSMK.R | 720 |
| C282 | pellino homolog 1 | | |
| Q8IY17_ | PNPLA6 Neuropathy target esterase | R.LAYVSC*VR.Q | 721 |
| C1199 | | | |
| Q8IY18_ | SMC5 Structural maintenance of | K.SSIVC*AICLGLAGKPAFMGR.A | 722 |
| C91 | chromosomes protein 5 | | |
| Q9NTJ3_ | SMC4 Structural maintenance of | R.FSC*IIGPNGSGK.S | 723 |
| C110 | chromosomes protein 4 | | |
| Q07065_ | CKAP4 Cytoskeleton-associated | K.SSSSSSASAAAAAAAASSSASC*SR.R | 724 |
| C100 | protein 4 | | |
| P51812_ | RPS6KA3 Ribosomal protein S6 | R.QGYDAAC*DIWSLGVLLYTMLTGYTPFA | 725 |
| C599 | kinase alpha-3 | NGPDDTPEEILAR.I | |
| Q86U44_ | METTL3 N6-adenosine- | K.GNPQGFNQGLDC*DVIVAEVR.S | 726 |
| C500 | methyltransferase 70 kDa subunit | | |
| Q9H479_ | FN3K Fructosamine-3-kinase | R.AFGGPGAGC*ISEGR.A | 727 |
| C24 | | | |
| Q9UPT9_ | USP22 Ubiquitin carboxyl-terminal | R.AIYQC*FVWSGTAEAR.K | 728 |
| C44 | hydrolase 22 | | |
| P30042_ | C21orf33 ES1 protein homolog, | K.VVTTPAFMC*ETALHYIHDGIGAMVR.K | 729 |
| C244 | mitochondrial | | |
| Q8NFY9_ | KBTBD8 Kelch repeat and BTB | R.IQGLAAVYKDSIYYIAGTC*GNHQR.M | 730 |
| C490 | domain-containing protein 8 | | |
| Q9UHQ1_ | NARF Nuclear prelamin A | K.VLVVSVC*PQSLPYFAAK.F | 731 |
| C99 | recognition factor | | |
| Q15042_ | RAB3GAP1 Rab3 GTPase-activating | K.IGC*PLTPLPPVSIAIR.F | 732 |
| C218 | protein catalytic subunit | | |
| Q9H0E9_ | BRD8 Bromodomain-containing | K.LC*LASSVMR.S | 733 |
| C26 | protein 8 | | |
| P51878_ | CASP5 Caspase-5 | K.VIIVQAC*R.G | 734 |
| C315 | | | |
| Q9UPQ9_ | TNRC6B Trinucleotide repeat- | R.SYRPTHPDC*QAVLQTLLSR.T | 735 |
| C600 | containing gene 6B protein | | |
| P27540_ | ARNT Aryl hydrocarbon receptor | K.MTAYITELSDMVPTC*SALAR.K | 736 |
| C119 | nuclear translocator | | |
| Q96C01_ | FAM136A Protein FAM136A | R.C*HVPLAQAQALVTSELEK.F | 737 |
| C57 | | | |
| Q9NQZ5_ | STARD7 StAR-related lipid transfer | R.YC*VSWMVSSGMPDFLEK.L | 738 |
| C302 | protein 7, mitochondria+ | | |
| Q9H9P8_ | L2HGDH L-2-hydroxyglutarate | K.AC*FLGATVK.Y | 739 |
| C376 | dehydrogenase, mitochondria+ | | |
| Q8NBX0_ | SCCPDH Saccharopine | R.WPISYC*R.E | 740 |
| C238 | dehydrogenase-like oxidoreductase | | |
| Q8NFF5_ | FLAD1 FAD synthase | R.TDPYSCSLC*PFSPTDPGWPAFMR.I | 741 |
| C499 | | | |
| Q6PJG6_ | BRAT1 BRCA1-associated ATM | R.C*QSPWTEALWVR.L | 742 |
| C228 | activator 1 | | |
| Q9C0C2_ | TNKS1BP1 182 kDa tankyrase-1- | K.DLQSEFGITGDPQPSSFSPSSWC*QGASQ | 743 |
| C749 | binding protein | DYGLOGASPR.G | |
| Q9Y4C1_ | KDM3A Lysine-specific demethylase | K.IVDPSLIHVEVVHDNLVTC*GNSAR.I | 744 |
| C251 | 3A | | |
| Q8N5C7_ | DTWD1 DTW domain-containing | K.IFTDERLQGLLQVELICTRKTC*FWRHQK. | 745 |
| C235 | protein 1 | G | |
| Q9BVP2_ | GNL3 Guanine nucleotide-binding | K.KLYC*QELK.K | 746 |
| C131 | protein-like 3 | | |
| Q6ZS81_ | WDFY4 WD repeat- and FYVE | R.DGKEPQPSAEAAAAPSLANISC*FTQK.L | 747 |
| C1963 | domain-containing protein 4 | | |
| O94829_ | IPO13 Importin-13 | R.TSLAVECGAVFPLLEQLLQQPSSPSC*VR. | 748 |
| C217 | | Q | |
| Q9BRT3_ | MIEN1 Migration and invasion | R.IVVEYCEPC*GFEATYLELASAVK.E | 749 |
| C33 | enhancer 1 | | |
| Q9BRJ7_ | NUDT16L1 Protein syndesmos | K.C*QLLFALK.V | 750 |
| C171 | | | |
| Q96NE9_ | FRMD6 FERM domain-containing | R.KLIYYTGC*PMR.S | 751 |
| C306 | protein 6 | | |
| Q70CQ2_ | USP34 Ubiquitin carboxyl-terminal | R.LATSAYDGC*SNSELCGMDQFWGIALR.A | 752 |
| C1090 | hydrolase 34 | | |
| O00622_ | CYR61 Protein CYR61 | TQPCDHTKK.TQPC*DHTK.G | 753 |
| C70 | | | |
| Q6VMQ6_ | ATF7IP Activating transcription | K.LCALQC*AVFDK.T | 754 |
| C612 | factor 7-interacting protein 1 | | |
| P68371_ | TUBB4B Tubulin beta-4B chain | K.VSDTVVEPYNATLSVHQLVENTDETYC*I | 755 |
| C213 | | DNEALYDIC*FR.T | |
| P04150_ | NR3C1 Glucocorticoid receptor | R.QSSANLLC*FAPDLIINEQR.M | 756 |
| C622 | | | |
| Q8WVB6_ | CHTF18 Chromosome transmission | R.SGEEEAAQPLGAPEEEPTDGQDASSHC*L | 757 |
| C280 | fidelity protein 18 homolog | WVDEFAPR.H | |
| Q7Z401_ | DENND4A C-myc promoter-binding | R.LWSSPAFSPTC*PFR.E | 758 |
| C1289 | protein | | |

Table 2 illustrates an exemplary list of liganded cysteines which are identified from isoTOP-ABPP experiments performed in situ. Table 2 further shows the accession number (or the protein identifier) of the protein, cysteine residue number, and an illustrative peptide fragment containing the cysteine of interest (denoted by C*).

TABLE 2

| | | | SEQ ID |
|---|---|---|---|
| Identifier | Protein Name | Illustrative Cysteine Fragment | NO: |
| P04406_ | GAPDH Glyceraldehyde-3-phosphate | K.IISNASC*TTNCLAPLAK.V | 759 |
| C152 | dehydrogenase | | |
| P61978_ | HNRNPK Heterogeneous nuclear | K.IIPTLEEGLQLPSPTATSQLPLESDAVE | 760 |
| C132 | ribonucleoprotein K | C*LNYQHYK.G | |
| Q13526_ | PIN1 Peptidyl-prolyl cis-trans | K.IKSGEEDFESLASQFSDC*SSAK.A | 761 |
| C113 | isomerase NIMA-interacting 1 | | |
| P24752_ | ACAT1 Acetyl-CoA acetyltransferase, | R.QAVLGAGLPISTPC*TTINK.V | 762 |
| C119 | mitochondrial | | |
| P24752_ | ACAT1 Acetyl-CoA acetyltransferase, | K.QGEYGLASIC*NGGGGASAMLIQK.L | 763 |
| C413 | mitochondrial | | |
| Q9NUY8_ | TBC1D23 TBC1 domain family member | K.FLENTPSSLNIEDIEDLFSLAQYYC*S | 764 |
| C283 | 23 | K.T | |
| P13667_ | PDIA4 Protein disulfide-isomerase A4 | K.ENFDEVVNDADIILVEFYAPWC*GHC | 765 |
| C206 | | K.K | |
| P12268_ | IMPDH2 Inosine-5-monophosphate | R.HGFC*GIPITDTGR.M | 766 |
| C140 | dehydrogenase 2 | | |
| Q15365_ | PCBP1 Poly(rC)-binding protein 1 | R.VMTIPYQPMPASSPVIC*AGGQDR.C | 767 |
| C194 | | | |
| Q9NVC6_ | MED17 Mediator of RNA polymerase II | K.MELLMSALSPC*LL | 768 |
| C649 | transcription subunit | | |
| P42166_ | TMPO Lamina-associated polypeptide | K.VDDEILGFISEATPLGGIQAASTESC* | 769 |
| C561 | 2, isoform alpha | NQQLDLALCR.A | |
| Q9Y696_ | CLIC4 Chloride intracellular channel | K.AGSDGESIGNC*PFSQR.L | 770 |
| C35 | protein 4 | | |
| P10599_ | TXN Thioredoxin | K.LVVVDFSATWC*GPCK.M | 771 |
| C32 | | | |
| P31943_ | HNRNPH1 Heterogeneous nuclear | R.DLNYC*FSGMSDHR.Y | 772 |
| C267 | ribonucleoprotein H | | |
| Q86SX6_ | GLRX5 Glutaredoxin-related protein | K.GTPEQPQC*GFSNAVVQILR.L | 773 |
| C67 | 5, mitochondrial | | |
| P15121_ | AKR1B1 Aldose reductase | R.VC*ALLSCTSHK.D | 774 |
| C299 | | | |
| P52597_ | HNRNPF Heterogeneous nuclear | R.DLSYC*LSGMYDHR.Y | 775 |
| C267 | ribonucleoprotein F | | |
| Q9ULV4_ | CORO1C Coronin-1C | K.KC*DLISIPK.K | 776 |
| C420 | | | |
| P62888_ | RPL30 60S ribosomal protein L30 | R.VC*TLAIIDPGDSDIIR.S | 777 |
| C92 | | | |
| Q9NQR4_ | NIT2 Omega-amidase NIT2 | R.VGLGIC*YDMR.F | 778 |
| C153 | | | |
| P42765_ | ACAA2 3-ketoacyl-CoA thiolase, | R.LC*GSGFQSIVNGCQEICVK.E | 779 |
| C92 | mitochondrial | | |
| Q15084_ | PDIA6 Protein disulfide-isomerase | R.EVIQSDSLWLVEFYAPWC*GHCQR.L | 780 |
| C55 | A6 | | |
| Q96HE7_ | ERO1L ERO1-like protein alpha | K.RPLNPLASGQGTSEENTFYSWLEGLC | 781 |
| C241 | | *VEK.R | |
| Q99439_ | CNN2 Calponin-2 | K.AGQC*VIGLQMGTNK.C | 782 |
| C164 | | | |
| P25205_ | MCM3 DNA replication licensing factor | R.TLTSC*FLSCVVCVEGIVTK.C | 783 |
| C119 | MCM3 | | |
| Q9NS86_ | LANCL2 LanC-like protein 2 | R.SVVC*QESDLPDELLYGR.A | 784 |
| C187 | | | |
| Q15233_ | NONO Non-POU domain-containing | R.FAC*HSASLTVR.N | 785 |
| C145 | octamer-binding protein | | |
| Q9BRA2_ | TXNDC17 Thioredoxin domain- | K.SWC*PDCVQAEPVVR.E | 786 |
| C43 | containing protein 17 | | |
| P35611_ | ADD1 Alpha-adducin | R.VSMILQSPAFC*EELESMIQEQFK.K | 787 |
| C68 | | | |
| O75521_ | ECI2 Enoyl-CoA delta isomerase 2, | R.WLSDEC*TNAVVNFLSR.K | 788 |
| C380 | mitochondrial | | |
| Q9BXW7_ | CECR5 Cat eye syndrome critical | R.DLC*FSPGLMEASHVVNDVNEAVQL | 789 |
| C392 | region protein 5 | VFR.K | |
| P30101_ | PDIA3 Protein disulfide-isomerase | K.SEPIPESNDGPVKVVVAENFDEIVNN | 790 |
| C406 | A3 | ENKDVLIEFYAPWC*GHCK.N | |
| Q96AB3_ | ISOC2 Isochorismatase domain- | R.SVLLCGIEAQAC*ILNTTLDLLDR.G | 791 |
| C114 | containing protein 2, mitochondrial | | |
| P13667_ | PDIA4 Protein disulfide-isomerase | K.KDVLIEFYAPWC*GHCK.Q | 792 |
| C555 | A4 | | |
| Q09161_ | NCBP1 Nuclear cap-binding protein | K.SAC*SLESNLEGLAGVLEADLPNYK. | 793 |
| C44 | subunit 1 | S | |
| P78417_ | GSTO1 Glutathione S-transferase | R.FC*PFAER.T | 794 |
| C32 | omega-1 | | |
| Q9ULW0_ | TPX2 Targeting protein for Xklp2 | R.TVEIC*PFSFDSR.D | 795 |
| C536 | | | |
| Q9NRG0_ | CHRAC1 Chromatin accessibility | K.ATELFVQC*LATYSYR.H | 796 |
| C55 | complex protein 1 | | |
| Q96T76_ | MMS19 MMS19 nucleotide excision | R.LMGLLSDPELGPAAADGFSLLMSDC | 797 |
| C848 | repair protein homolog | *TDVLTR.A | |
| Q8TAQ2_ | SMARCC2 SWI/SNF complex subunit | K.SLVQNNCLSRPNIFLC*PEIEPK.L | 798 |
| C145 | SMARCC2 | | |
| Q9BVC5_ | C2orf49 Ashwin | R.SC*TDSELLLHPELLSQEFLLLTLEQK. | 799 |
| C10 | | N | |
| Q7Z2W4_ | ZC3HAV1 Zinc finger CCCH-type | K.NSNVDSSYLESLYQSC*PR.G | 800 |
| C645 | antiviral protein 1 | | |
| Q9BQ69_ | MACROD1 O-acetyl-ADP-ribose | K.LEVDAIVNAANSSLLGGGGVDGC*IH | 801 |
| C186 | deacetylase MACRODI | R.A | |
| Q16831_ | UPP1 Uridine phosphorylase 1 | R.IGTSGGIGLEPGTVVITEQAVDTC*FK. | 802 |
| C162 | | A | |
| P30101_ | PDIA3 Protein disulfide-isomerase | R.ISDTGSAGLMLVEFFAPWC*GHCK.R | 803 |
| C571 | A3 | | |
| P12268_ | IMPDH2 Inosine-5-monophosphate | R.VGMGSGSIC*ITQEVLACGRPQATAV | 804 |
| C331 | dehydrogenase 2 | YK.V | |
| O95571_ | ETHE1 Protein ETHE1, mitochondrial | R.TDFQQGC*AK.T | 805 |
| C170 | | | |
| O00299_ | CLIC1 Chloride intracellular channel | K.IGNC*PFSQR.L | 806 |
| C24 | protein 1 | | |
| O14879_ | IFIT3 Interferon-induced protein | K.GLNPLNAYSDLAEFLETEC*YQTPFN | 807 |
| C343 | with tetratricopeptide | K.E | |
| Q96CM8_ | ACSF2 Acyl-CoA synthetase family | R.MVSTPIGGLSYVQGC*TK.K | 808 |
| C64 | member 2, mitochondrial | | |
| P51946_ | CCNH Cyclin-H | R.TC*LSQLLDIMK.S | 809 |
| C244 | | | |
| P49588_ | AARS Alanine-tRNA ligase, | K.C*LSVMEAK.V | 810 |
| C773 | cytoplasmic | | |
| Q96RN5_ | MED15 Mediator of RNA polymerase II | K.QQYLC*QPLLDAVLANIR.S | 811 |
| C618 | transcription subunit | | |
| O15294_ | OGT UDP-N-acetylglucosarnine-peptide | K.C*PDGGDNADSSNTALNMPVIPMNTI | 812 |
| C758 | N-acetylglucosaminyl transferase | AEAVIEMINR.G | |
| P46734_ | MAP2K3 Dual specificity mitogen- | K.MC*DEGISGYLVDSVAK.T | 813 |
| C207 | activated protein kinase | | |
| Q96S55_ | WRNIP1 ATP ase WRNIP1 | R.SLLETNELPSLILWGPPGC*GK.T | 814 |
| C272 | | | |
| O95229_ | ZWINT ZW10 interactor | K.LLC*SQLQVADFLQNILAQEDTAK.G | 815 |
| C54 | | | |
| O60610_ | DIAPH1 Protein diaphanous homolog 1 | K.AGC*AVTSLLASELTK.D | 816 |
| C1227 | | | |
| Q13428_ | TCOF1 Treacle protein | K.C*FLAQPVTLLDIYTHWQQTSELGR. | 817 |
| C38 | | K | |
| Q9Y277_ | VDAC3 Voltage-dependent anion- | K.VC*NYGLTFTQK.W | 818 |
| C65 | selective channel protein | | |
| P57764_ | GSDMD Gasdermin-D | R.C*LHNFLTDGVPAEGAFTEDFQGLR. | 819 |
| C268 | | A | |
| Q9Y3A3_ | MOB4 MOB-like protein phocein | R.HTLDGAAC*LLNSNK.Y | 820 |
| C134 | | | |
| Q02252_ | ALDH6A1 Methylmalonate-semialdehyde | R.C*MALSTAVLVGEAKIC | 821 |
| C317 | dehydrogenase | | |
| Q9NYL9_ | TMOD3 Tropomodulin-3 | K.VSLDPELEEALTSASDTELC*DLAAIL | 822 |
| C132 | | GMHNLITNTK.F | |
| P83731_ | RPL24 60S ribosomal protein L24 | K.VELC*SFSGYK.I | 823 |
| C6 | | | |
| O95336_ | PGLS 6-phosphogluconolactonase | R.AAC*CLAGAR.A | 824 |
| C32 | | | |
| Q13155_ | AIMP2 Aminoacyl tRNA synthase | K.SPWLAGNELTVADVVLWSVLQQIGG | 825 |
| C291 | complex-interacting multif | C*SVTVPANVQR.W | |
| Q13418_ | ILK Integrin-linked protein kinase | K.FSFQC*PGR.M | 826 |
| C346 | | | |
| A6NDU8_ | C5orf51 UPF0600 protein C5orf51 | R.C.PIQLNEGVSFQDLDTAK.L | 827 |
| C179 | | | |
| Q9UKF6_ | CPSF3 Cleavage and polyadenylation | R.NFNYHILSPC*DLSNYTDLAMSTVK. | 828 |
| C498 | specificity factor subunit | Q | |
| Q96F86_ | EDC3 Enhancer of mRNA-decapping | K.DLPTSPVDLVINCLDC*PENVFLR.D | 829 |
| C413 | protein 3 | | |
| P42224_ | STAT1 Signal transducer and | R.NLSFFLTPPC*AR.W | 830 |
| C492 | activator of transcription 1 | | |
| P11216_ | PYGB Glycogen phosphorylase, brain | R.TC*FETFPDK.V | 831 |
| C326 | form | | |
| P21980_ | TGM2 Protein-glutamine gamma- | K.YGQC*WVFAAVACTVLR.C | 832 |
| C277 | glutamyltransferase 2 | | |
| Q9HAV7_ | GRPEL1 GrpE protein homolog 1, | K.ATQC*VPKEEIICDDNPIILK.N | 833 |
| C124 | mitochondrial | | |
| P24752_ | ACAT1 Acetyl-CoA acetyltransferase, | K.VC*ASGMK.A | 834 |
| C126 | mitochondrial | | |
| Q9NQ88_ | TIGAR Fructose-2,6-bisphosphatase | K.EADQKEQFSQGSPSNC*LETSLAEIFP | 835 |
| C161 | TIGAR | LGK.N | |
| Q13155_ | AIMP2 Aminoacyl tRNA synthase | R.VELPTC*MYR.L | 836 |
| C23 | complex-interacting multif | | |
| Q9NQW6_ | ANLN Actin-binding protein anillin | K.NNAFPC*QVNIK.Q | 837 |
| C712 | | | |
| P51649_ | ALDH5A1 Succinate-semialdehyde | R.NTGQTC*VCSNQFLVQR.G | 838 |
| C340 | dehydrogenase, mitochondrial | | |
| Q15021_ | NCAPD2 Condensin complex subunit 1 | K.NAIQLLASFLANNPFSC*K.L | 839 |
| C439 | | | |
| Q5T0N5_ | FNBP1L Formin-binding protein 1-like | R.FTSC*VAFFNILNELNDYAGQR.E | 840 |
| C69 | | | |
| P38606_ | ATP6V1A V-type proton ATPase | KMDFTPC*K.N | 841 |
| C138 | catalytic subunit A | | |
| Q9HCC0_ | MCCC2 Methylcrotonoyl-CoA | K.NIAQIAVVMGSC*TAGGAYVPAMAD | 842 |
| C216 | carboxylase beta chain, mitoch | ENTIVR.K | |
| Q9NQC3_ | RTN4 Reticulon-4 | K.YSNSALGHVNC*TIK.E | 843 |
| C1101 | | | |
| P35754_ | GLRX Glutaredoxin-1 | K.VVVFIKPTC*PYCR.R | 844 |
| C23 | | | |
| Q99757_ | TXN2 Thioredoxin, mitochondrial | R.VVNSETPVVVDFHAQWC*GPCK.I | 845 |
| C90 | | | |
| Q9Y3D0_ | FAM96B Mitotic spindle-associated | R.VQVSDPESTVAVAFTPTIPHC*SMAT | 846 |
| C93 | MMXD complex subunit MI | LIGLSIK.V | |
| Q9UMS0_ | NFU1 NFU1 iron-sulfur cluster | K.LQGSCTSC*PSSIITLK.N | 847 |
| C213 | scaffold homolog, mitochondrial | | |
| Q9NXV6_ | CDKN2AIP CDKN2A-interacting protein | K.SVYLGTGC*GK.S | 848 |
| C516 | | | |
| Q96RS6_ | NUDCD1 NudC domain-containing | R.DSAQC*AAIAER.L | 849 |
| C376 | protein 1 | | |
| Q14997_ | PSME4 Proteasome activator complex | K.QQFTDDQLLVLTDLLVSPC*YYA | 850 |
| C1840 | subunit 4 | | |
| P50570_ | DNM2 Dynamin-2 | K.LQDAFSSIGQSC*HLDLPQIAVVGGQ | 851 |
| C27 | | SAGK.S | |
| Q86YH6_ | PDSS2 Decaprenyl-diphosphate | R.C*LLSDELSNIAMQVR.K | 852 |
| C71 | synthase subunit 2 | | |
| Q99497_ | PARK7 Protein DJ-1 | K.GLIAAIC*AGPTALLAHEIGFGSK.V | 853 |
| C106 | | | |
| Q9UJW0_ | DCTN4 Dynactin subunit 4 | R.LLQPDFQPVC*ASQLYPR.H | 854 |
| C258 | | | |
| Q9BUH6_ | C9orf142 Uncharacterized protein | R.C*PGESLINPGFK.S | 855 |
| C180 | C9orf142 | | |
| P24752_ | ACAT1 Acetyl-CoA acetyltransferase, | K.IHMGSC*AENTAK.K | 856 |
| C196 | mitochondrial | | |
| Q13162_ | PRDX4 Peroxiredoxin-4 | R.EEEC*HFYAGGQVYPGEASR.V | 857 |
| C51 | | | |
| Q9BTA9_ | WAC WW domain-containing adapter | R.STC*SLTPALAAHFSENLIK.H | 858 |
| C553 | protein with coiled-coil | | |
| P48643_ | CCT5 T-complex protein 1 subunit | K.IAILTC*PFEPPKPK.T | 859 |
| C253 | epsilon | | |
| O75362_ | ZNF217 Zinc finger protein 217 | R.C*IPQLDPFTTFQAWQLATK.G | 860 |
| C286 | | | |
| O60825_ | PFKFB2 6-phosphofructo-2-kinase/ | K.VFFVESVC*DDPDVIAANILEVK.V | 861 |
| C158 | fructose-2,6-bisphosphatase 2 | | |
| Q8NBS9_ | TXNDC5 Thioredoxin domain-containing | K.FYAPWC*GHCK.T | 862 |
| C350 | protein 5 | | |
| Q9NYL2_ | MLTK Mitogen-activated protein kinase | K.FDDLQFFENC*GGGSFGSVYR.A | 863 |
| C22 | kinase kinase MLT | | |
| P27707_ | DCK Deoxycytidine kinase | R.SC*PSFSASSEGTR.I | 864 |
| C9 | | | |
| Q93009_ | USP7 Ubiquitin carboxyl-terminal | K.NQGATC*YMNSLLQTLFFTNQLR.K | 865 |
| C223 | hydrolase 7 | | |
| O14929_ | HAT1 Histone acetyltransferase type | K.VDENFDC*VEADDVEGKJ | 866 |
| C101 | B catalytic subunit | | |
| Q9UPQ0_ | LIMCH1 LIM and calponin homology | K.AANSC*TSYSGTTLNLK.E | 867 |
| C140 | domains-containing protein | | |
| Q96NY7_ | CLIC6 Chloride intracellular channel | R.AGYDGESIGNC*PFSQR.L | 868 |
| C487 | protein 6 | | |
| Q9NQ88_ | TIGAR Fructose-2,6-bisphosphatase | R.EEC*PVFTPPGGETLDQVK.M | 869 |
| C114 | TIGAR | | |
| Q14790_ | CASP8 Caspase-8 | K.VFFIQAC*QGDNYQK.G | 870 |
| C360 | | | |
| P04183_ | TK1 Thymidine kinase, cytosolic | K.LFAPQQILQC*SPAN.- | 871 |
| C230 | | | |
| P68366_ | TUBA4A Tubulin alpha-4A chain | K.TIGGGDDSFTTFFC*ETGAGK.H | 872 |
| C54 | | | |
| Q13428_ | TCOF1 Treacle protein | K.GAGNPQASTLALQSNITQC*LLGQP | 873 |
| C1298 | | WPLNEAQVQASVVK.V | |
| Q5MNZ6_ | WDR45L WD repeat domain | R.C*NYLALVGGGK.K | 874 |
| C63 | phosphoinositide-interacting protein | | |
| O14980_ | XPO1 Exportin-1 | K.DLLGLC*EQK.R | 875 |
| C528 | | | |
| Q86W42_ | THOC6 THO complex subunit 6 homolog | R.LHMTIFSQSVSPC*GK.F | 876 |
| C35 | | | |
| Q9Y6G9_ | DYNC1LI1 Cytoplasmic dynein 1 light | R.VGSFGSSPPGLSSTYTGGPLGNEIASG | 877 |
| C51 | intermediate chain 1 | NGGAAAGDDEDGQNLWSC*ILSEVSTR | |
| | | .S | |
| Q9NY27_ | PPP4R2 Serine/threonine-protein | K.EVC*PVLDQFLCHVAK.T | 878 |
| C22 | phosphatase 4 regulatory | | |
| Q8NFH5_ | NUP35 Nucleoporin NUP53 | R.C*ALSSPSLAFTPPIK.T | 879 |
| C255 | | | |
| Q9Y676_ | MRPS18B 28S ribosomal protein S18b, | K.LLEQFVC*AHTGIIFYAPYTGVCVK.Q | 880 |
| C128 | mitochondrial | | |
| P35658_ | NUP214 Nuclear pore complex protein | K.AC*FQVGTSEEMK.M | 881 |
| C728 | Nup214 | | |
| Q9NTX5_ | ECHDC1 Ethylmalonyl-CoA | K.SLGTPEDGMAVC*MFMQNTLTR.F | 882 |
| C133 | decarboxylase | | |
| Q15118_ | PDK1 | K.QFLDFGSVNAC*EK.T | 883 |
| C71 | | | |
| Q00765_ | REEP5 Receptor expression-enhancing | K.NC*MTDLLAK.L | 884 |
| C18 | protein 5 | | |
| P22307_ | SCP2 Non-specific lipid-transfer | K.ALADAQIPYSAVDQACVGYVFGDST | 885 |
| C71 | protein | C*GQR.A | |
| O75521_ | ECI2 Enoyl-CoA delta isomerase 2, | K.KLTAGEAC*AQGLVTEVFPDSTFQK. | 886 |
| C312 | mitochondrial | E | |
| P49189_ | ALDH9A1 4- | K.GALMANFLTQGQVC*CNGTR.V | 887 |
| C288 | trimethylaminobutyraldehyde | | |
| | dehydrogenase | | |
| Q5T440_ | IBA57 Putative transferase CAF17, | R.VWAVLPSSPEAC*GAASLQER.A | 888 |
| C170 | mitochondrial | | |
| Q15084_ | PDIA6 Protein disulfide-isomerase A6 | K.DVIELTDDSFDKNVLDSEDVWMVEF | 889 |
| C190 | | YAPWC*GHCK.N | |
| Q96C19_ | EFHD2 EF-hand domain-containing | K.AAAGELQEDSGLC*VLAR.L | 890 |
| C172 | protein D2 | | |
| P22061_ | PCMT1 Protein-L-isoaspartate | R.MVGC*TGK.V | 891 |
| C102 | (D-aspartate) O-methyltransferase | | |
| Q9NP73_ | ALG13 UDP-N-acetylglucosamine | K.ADLVISHAGAGSC*LETLEK.G | 892 |
| C86 | transferase subunit ALG13 | | |
| Q9BRF8_ | CPPED1 Calcineurin-like | K.AWSTGDC*DNGGDEWEQEIR.L | 893 |
| C54 | phosphoesterase domain-containing | | |
| Q6ICB0_ | DESI1 Desumoylating isopeptidase 1 | R.GEAYNLFEHNC*NTFSNEVAQFLTGR | 894 |
| C108 | | .K | |
| P29590_ | PML Protein PML | R.LQDLSSC*ITQGK.D | 895 |
| C389 | | | |
| P07858_ | CTSB Cathepsin B | K.IC*EPGYSPTYK.Q | 896 |
| C211 | | | |
| Q9NX18_ | SDHAF2 Succinate dehydrogenase | R.GMLENC*ILLSLFAK.E | 897 |
| C83 | assembly factor 2, mitochondrial | | |
| P46109_ | CRKL Crk-like protein | K.RVPC*AYDK.T | 898 |
| C249 | | | |
| P45984_ | MAPK9 Mitogen-activated protein | R.TAC*INFMMTPYVVTR.Y | 899 |
| C177 | kinase 9 | | |
| P19447_ | ERCC3 TFIIH basal transcription | R.SGVIVLPC*GAGK.S | 900 |
| C342 | factor complex helicase | | |
| P42166_ | TMPO Lamina-associated polypeptide | K.SGIQPLC*PER.S | 901 |
| C341 | 2, isoform alpha | | |
| Q8N1F7_ | NUP93 Nuclear pore complex protein | K.SSGQSAQLLSHEPGDPPC*LR.R | 902 |
| C522 | Nup93 | | |
| Q86UY8_ | NT5DC3 5-nucleotidase domain- | K.YIC*YAEQTR.A | 903 |
| C276 | containing protein 3 | | |
| Q8WWI1_ | LMO7 LIM domain only protein 7 | R.DSGYGDIWC*PER.G | 904 |
| C228 | | | |
| Q9NWA0_ | MED9 Mediator of RNA polymerase II | K.SLC*MFEIPKE | 905 |
| C139 | transcription subunit | | |
| P09110_ | ACAA1 3-ketoacyl-CoA thiolase, | K.VNPLGGAVALGHPLGC*TGAR.Q | 906 |
| C381 | peroxisomal | | |
| Q2NL82_ | TSR1 Pre-rRNA-processing protein | R.DTGTVHLNELGNTQNFMLLC*PR.L | 907 |
| C126 | TSR1 homolog | | |
| Q5JPI3_ | C3orf38 Uncharacterized protein | K.FEQSDLEAFYNVITVC*GTNEVR.H | 908 |
| C308 | C3orf38 | | |
| P23919_ | DTYMK Thymidylate kinase | R.C*FHQLMK.D | 909 |
| C163 | | | |
| Q96EB1_ | ELP4 Elongator complex protein 4 | K.VEPC*SLTPGYTK.L | 910 |
| C218 | | | |
| Q96FX7_ | TRMT61A tRNA (adenine(58)-N(1))- | R.FCSFSPC*TEQVQR.T | 911 |
| C209 | methyltransferase catalytic | | |
| | subunit TRMT61A | | |
| O14933_ | UBE2L6 Ubiquitin/ISG15-conjugating | K.IYHPNVDENGQICLPIISSENWKPC*T | 912 |
| C98 | enzyme E2 L6 | K.T | |
| Q29RF7_ | PDS5A Sister chromatid cohesion | R.TVQTIEAC*IANFFNQVLVLGR.S | 913 |
| C242 | protein PDS5 homolog A | | |
| Q96T76_ | MMS19 MMS19 nucleotide excision | R.YHPLSSC*LTAR.L | 914 |
| C819 | repair protein homolog | | |
| P23919_ | DTYMK Thymidylate kinase | K.ENFSLDWC*K.Q | 915 |
| C117 | | | |
| Q15149_ | PLEC Plectin | K.YLTC*PK.T | 916 |
| C4574 | | | |
| Q96RP9_ | GFM1 Elongation factor G, | R.VLDGAVLVLCAVGGVQC*QTMTVN | 917 |
| C153 | mitochondrial | R.Q | |
| P04818_ | TYMS Thymidylate synthase | R.DLPLMALPPCHALC*QFYVVNSELSC | 918 |
| C199 | | QLYQR.S | |
| P27708_ | CAD CAD protein | K.AQILVLTYPLIGNYGIPPDEMDEFGL | 919 |
| C73 | | C*K.W | |
| P55265_ | ADAR Double-stranded RNA-specific | K.NFYLC*PV | 920 |
| C1224 | adenosine deaminase | | |
| Q9Y3D2_ | MSRB2 Methionine-R-sulfoxide | K.YC*SGTGWPSFSEAHGTSGSDESHTG | 921 |
| C105 | reductase B2, mitochondrial | ILR.R | |
| O00244_ | ATOX1 Copper transport protein ATOX1 | K.HEFSVDMTC*GGCAEAVSR.V | 922 |
| C12 | | | |
| Q8WV74_ | NUDT8 Nucleoside diphosphate-linked | R.LAGLTC*SGAEGLARPK.Q | 923 |
| C207 | moiety X motif 8, mitochondrial | | |
| Q9NRW3_ | APOBEC3C Probable DNA dC-dU-editing | R.LYYFQYPC*YQEGLR.S | 924 |
| C130 | enzyme APOBEC-3C | | |
| P24468_ | NR2F2 COUP transcription factor 2 | K.AIVLFTSDAC*GLSDVAHVESLQEK.S | 925 |
| C326 | | | |
| P42166_ | TMPO Lamina-associated polypeptide | K.GGTLFGGEVC*K.V | 926 |
| C684 | 2, isoform alpha | | |
| Q96EY5_ | FAM125A Multivesicular body subunit | K.SC*SPLAFSAFGDLTIK.S | 927 |
| C231 | 12A | | |
| P14635_ | CCNB1 G2/mitotic-specific cyclin-B1 | R.FMQNNC*VPK.K | 928 |
| C238 | | | |
| Q8NDH3_ | NPEPL1 Probable aminopeptidase | R.VTEELWQAALSTLNPNPTDSCPLYLN | 929 |
| C81 | NPEPL1 | YATVAALPC*R.V | |
| Q9P0J1_ | PDP1 | R.GMLLGVFDGHAGC*ACSQAVSER.L | 930 |
| C149 | | | |
| Q96P48_ | ARAP1 Arf-GAP with Rho-GAP domain, | R.AVFPEGPC*EEPLQLR.K | 931 |
| C900 | ANK repeat and PH domain-containing | | |
| | protein 1 | | |
| Q96HE7_ | ERO1L ERO1-like protein alpha | R.CFC*QVSGYLDDCTCDVETIDR.F | 932 |
| C37 | | | |
| Q07065_ | CKAP4 Cytoskeleton-associated | K.SSSSSSASAAAAAAAASSSASC*SR.R | 933 |
| C100 | protein 4 | | |
| Q9BRJ7_ | NUDT16L1 Protein syndesmos | R.VLGLGLGC*LR.L | 934 |
| C88 | | | |
| O75439_ | PMPCB Mitochondrial-processing | K.FHFGDSLC*THK.G | 935 |
| C265 | peptidase subunit beta | | |
| O43175_ | PHGDH D-3-phosphoglycerate | K.NAGNC*LSPAVIVGLLK.E | 936 |
| C369 | dehydrogenase | | |
| Q9UNI6_ | DUSP12 Dual specificity protein | R.QAQC*TSYFIEPVQWMESALLGVMD | 937 |
| C265 | phosphatase 12 | GQLLCPK.C | |
| Q06203_ | PPAT Amidophosphoribosyltransferase | K.C*ELENCQPFVVETLHGKI | 938 |
| C100 | | | |
| A0AVT1_ | UBA6 Ubiquitin-like modifier- | R.KPNVGC*QQDSEELLK.L | 939 |
| C347 | activating enzyme 6 | | |
| Q86X76_ | NIT1 Nitrilase homolog 1 | KIGLAVC*YDMR.F | 940 |
| C203 | | | |
| Q6XZE7_ | DNMBP Dynamin-binding protein | R.SLDQTSPC*PLVLVR.I | 941 |
| C691 | | | |
| Q15398_ | DLGAP5 Disks large-associated | R.YRPDMPC*FLLSNQNAVK.A | 942 |
| C129 | protein 5 | | |
| O75717_ | WDHD1 WD repeat and HMG-box | CK.MLALSC*K.L | 943 |
| 773 | DNA-binding protein 1 | | |
| Q01433_ | AMPD2 AMP deaminase 2 | R.SLPGPAPC*LK.H | 944 |
| C107 | | | |
| Q8WVV9_ | HNRPLL Heterogeneous nuclear | K.NIIQPPSCVLHYYNVPLC*VTEETFTK | 945 |
| C464 | ribonucleoprotein L-like | .L | |
| O14733_ | MAP2K7 Dual specificity mitogen- | R.YQAEINDLENLGEMGSGTC*GQVWK | 946 |
| C131 | activated protein kinase | .M | |
| Q14137_ | BOP1 Ribosome biogenesis protein | R.DLQPFPTC*QALVYR.G | 947 |
| C404 | BOP1 | | |
| Q96RU2_ | USP28 Ubiquitin carboxyl-terminal | K.NVGNTC*WFSAVIQSLFQLPEFR.R | 948 |
| C171 | hydrolase 28 | | |
| Q9Y679_ | AUP1 Ancient ubiquitous protein 1 | K.TGC*VDLTITNLLEGAVAFMPEDITK | 949 |
| C391 | | G | |
| P51610_ | HCFC1 Host cell factor 1 | R.VAGINAC*GR.G | 950 |
| C1872 | | | |
| P22307_ | SCP2 Non-specific lipid-transfer | K.SGLTPNDIDVIELHDC*FSTNELLTYE | 951 |
| C307 | protein | ALGLCPEGQGATLVDR.G | |
| Q9BTE3_ | MCMBP Mini-chromosome maintenance | K.LQHINPLLPAC*LNK.E | 952 |
| C325 | complex-binding protein | | |
| Q9HA64_ | FN3KRP Ketosamine-3-kinase | R.ATGHSGGGC*ISQGR.S | 953 |
| C24 | | | |
| Q5TFE4_ | NT5DC1 5-nucleotidase domain- | K.HFLSDTGMAC*R.S | 954 |
| C119 | containing protein 1 | | |
| Q96N67_ | DOCK7 Dedicator of cytokinesis | K.AVLPVTC*HR.D | 955 |
| C2125 | protein 7 | | |
| P52948_ | NUP98 Nuclear pore complex protein | R.WLSC*TATPQLEEEVSLTQK.N | 956 |
| C1312 | Nup98-Nup96 | | |
| Q5U1P0_ | RIF1 Telomere-associated protein | K.ESIPCPTESVYPPLVNC*VAPVDIELPQ | 957 |
| C2298 | RIF1 | ITSNMWAR.G | |
| P51812_ | RPS6KA3 Ribosomal protein S6 kinase | K.EDIGVGSYSVC*K.R | 958 |
| C436 | alpha-3 | | |
| Q92616_ | GCN1L1 Translational activator GCN1 | K.GMGESC*FEDLLPWLMETLTYEQSS | 959 |
| C1692 | | VDR.S | |
| Q15345_ | LRRC41 Leucine-rich repeat-containing | R.C*AAALMASR.R | 960 |
| C297 | protein 41 | | |
| Q9NPH0_ | ACP6 Lysophosphatidic acid | R.MGIDSSDKVDFFILLDNVAAEQAHN | 961 |
| C267 | phosphatase type 6 | LPSC*PMIX.R | |
| P04183_ | TK1 Thymidine kinase, cytosolic | R.YSSSFC*THDR.N | 962 |
| C66 | | | |
| P42166_ | TMPO Lamina-associated polypeptide 2, | K.TYDAASYIC*EAAFDEVK.M | 963 |
| C629 | isoform alpha | | |
| Q15013_ | MAD2L1BP MAD2L1-binding protein | K.C*QQALAELESVLSHLEDFFAR.T | 964 |
| C124 | | | |
| Q9Y5Y2_ | NUBP2 Cytosolic Fe-S cluster assembly | R.AVHQC*DR.G | 965 |
| C72 | factor NUBP2 | | |
| O15446_ | CD3EAP DNA-directed RNA polymerase | R.VLSSC*PQAGEATLLAPSTEAGGGLT | 966 |
| C86 | 1 subunit RPA34 | CASAPQGTLR.I | |
| Q13630_ | TSTA3 GDP-L-fucose synthase | R.KVVSCLSTC*IFPDK.T | 967 |
| C116 | | | |
| Q8IYQ7_ | THNSL1 Threonine synthase-like 1 | R.LGEMIETAYGENFAC*SK.I | 968 |
| C324 | | | |
| P05091_ | ALDH2 Aldehyde dehydrogenase, | K.SPNIIMSDADMDWAVEQAILFALFFN | 969 |
| C319 | mitochondrial | QGQCC*CAGSR.T | |
| Q29RF7_ | PDS5A Sister chromatid cohesion | R.ELLDLHKQPTSEANC*SAMFGK.L | 970 |
| C532 | protein PDS5 homolog A | | |
| Q9Y570_ | PPME1 Protein phosphatase | R.FAEPIGGFQC*VFPGC.- | 971 |
| C381 | methylesterase 1 | | |
| Q14980_ | NUMA1 Nuclear mitotic apparatus | R.QFC*STQAALQAMER.E | 972 |
| C961 | protein 1 | | |
| P53384_ | NUBP1 Cytosolic Fe-S cluster | K.LPHGVVENMSGFIC*PK.C | 973 |
| C235 | assembly factor NUBP1 | | |
| Q15003_ | NCAPH Condensin complex subunit 2 | R.TMC*PLLSMKPGEYSYFSPR.T | 974 |
| C418 | | | |
| P53634_ | CTSC Dipeptidyl peptidase 1 | R.NQASCGSC*YSFASMGMLEARI | 975 |
| C258 | | | |
| Q8NFF5_ | FLAD1 FAD synthase | R.TDPYSCSLC*PFSPTDPGWPAFMR.I | 976 |
| C499 | | | |
| Q9ULA0_ | DNPEP Aspartyl aminopeptidase | K.C*PTSGR.L | 977 |
| C144 | | | |
| P22307_ | SCP2 Non-specific lipid-transfer | R.AIYHSLGMTGIPIENVININNC*ATGSTA | 978 |
| C94 | protein | LFMAR.Q | |
| O15294_ | OGT UDP-N-acetylglucosamine--peptide | K.VMAEANHFIDLSQIPC*NGK.A | 979 |
| C620 | N-acetylglucosaminyl transferase | | |
| Q9Y5S2_ | CDC42BPB Serine/threonine-protein | R.IRPLNSEGTLNLLNC*EPPR.L | 980 |
| C1517 | kinase MRCK beta | | |
| Q8TD19_ | NEK9 Serine/threonine-protein kinase | R.LLTFGC*NK.C | 981 |
| C623 | Nek9 | | |
| Q8N2W9_ | PIAS4 E3 SUMO-protein ligase PIAS4 | R.VSLIC*PLVK.M | 982 |
| C326 | | | |
| Q13158_ | FADD Protein FADD | R.VDDFEAGAAAGAAPGEEDLC*AAFN | 983 |
| C98 | | VICDNVGK.D | |
| Q9UKX7_ | NUP50 Nuclear pore complex protein | K.AC*VGNAYHK.Q | 984 |
| C151 | Nup50 | | |
| Q6PCB5_ | RSBN1L Round spermatid basic protein | K.SIQTIC*SGLLTDVEDQAAK.G | 985 |
| C280 | 1-like protein | | |
| P10398_ | ARAF Serine/threonine-protein kinase | R.TQADELPAC*LLSAAR.L | 986 |
| C597 | A-Raf | | |
| Q9UL40_ | ZNF346 Zinc finger protein 346 | K.NQC*LFTNTQCK.V | 987 |
| C68 | | | |
| P46013_ | MIC167 Antigen KI-67 | K.SEETNTEIVEC*ILK.R | 988 |
| C903 | | | |
| Q16667_ | CDICN3 Cyclin-dependent kinase | R.VNC*SQFLGLCALPGCK.F | 989 |
| C39 | inhibitor 3 | | |
| O75150_ | RNF40 E3 ubiquitin-protein ligase | R.EIQPC*LAESR.A | 990 |
| C890 | BRE1B | | |
| Q00610_ | CLTC Clathrin heavy chain 1 | RIHEGC*EEPATHNALAK.I | 991 |
| C870 | | | |
| Q9Y5T5_ | USP16 Ubiquitin carboxyl-terminal | K.GLSNLGNTC*FFNAVMQNLSQTPVL | 992 |
| C205 | hydrolase 16 | R.E | |
| O95881_ | TXNDC12 Thioredoxin domain-containing | K.SWC*GACK.A | 993 |
| C66 | protein 12 | | |
| Q7Z5K2_ | WAPAL Wings apart-like protein | R.IVEDDASISSC*NK.L | 994 |
| C160 | homolog | | |
| P42166_ | TMPO Lamina-associated polypeptide 2, | R.QLPSLAC*K.Y | 995 |
| C518 | isoform alpha | | |
| Q9Y2S7_ | POLD1P2 Polymerase delta-interacting | R.DC*PHISQR.S | 996 |
| C143 | protein 2 | | |
| E2QRD5_ | C15orf38-AP3S2 Protein C15orf38- | K.C*NFTGDGICT | 997 |
| C183 | AP3S2 | | |
| O95833_ | CLIC3 Chloride intracellular channel | K.ASEDGESVGHC*PSCQR.L | 998 |
| C22 | protein 3 | | |
| O94953_ | KDM4B Lysine-specific demethylase 4B | R.TEPYCAICTLFYPYC*QALQTEK.E | 999 |
| C694 | | | |
| O00541_ | PES1 Pescadillo homolog | K.AGEGTYALDSESC*MEK.L | 1000 |
| C272 | | | |
| Q9NXJ5_ | PGPEP1 Pymglutamyl-peptidase 1 | R.YLC*DFTYYTSLYQSHGR.S | 1001 |
| C149 | | | |
| Q8N5L8_ | RPP25L Ribonuclease P protein subunit | R.DPLDPNEC*GYQPPGAPPGLGSMPSS | 1002 |
| C131 | p25-like protein | SCGPR.S | |
| Q8IZ73_ | RPUSD2 RNA pseudouridylate synthase | R.LLAENEDVVVVDKPSSIPVHPC*GR.F | 1003 |
| C246 | domain-containing protein | | |
| Q99798_ | ACO2 Aconitate hydratase, | R.VGLIGSC*TNSSYEDMGR.S | 1004 |
| C385 | mitochondrial | | |
| Q9GZR2_ | REXO4 RNA exonuclease 4 | K.ILGLQVQQAEHC*SIQDAQAAMR.L | 1005 |
| C382 | | | |
| Q13613_ | MTMR1 Myotubularin-related protein 1 | K.DVMYIC*PFMGAVSGTLTVTDFK.L | 1006 |
| C117 | | | |
| Q9NUI1_ | DECR2 Peroxisomal 2,4-dienoyl-CoA | R.HLFC*PDLLR.D | 1007 |
| C22 | reductase | | |
| Q02556_ | IRF8 Interferon regulatory factor 8 | R.VFCSGNAVVC*K.G | 1008 |
| C306 | | | |
| Q9UPT9_ | USP22 Ubiquitin carboxyl-terminal | K.ITSNC*TIGLR.G | 1009 |
| C171 | hydrolase 22 | | |
| Q8N999_ | Cl2orf29 Uncharacterized protein | K.C*LFNHFLK.I | 1010 |
| C302 | C12orf29 | | |
| Q8IU81_ | IRF2BP1 Interferon regulatory factor | R.SFREPAPAEALPQQYPEPAPAALC*G | 1011 |
| C363 | 2-binding protein 1 | PPPR.A | |
| Q9C0I1_ | MTMR12 Myotubularin-related | R.VFQFC*LR.Y | 1012 |
| C152 | protein 12 | | |
| Q9P2X3_ | IMPACT Protein IMPACT | R.STFQAHLAPVVC*PK.Q | 1013 |
| C195 | | | |
| Q6QNY0_ | BLOC1S3 Biogenesis of lysosome | R.GDLC*ALAER.L | 1014 |
| C168 | related organelles complex | | |
| Q15796_ | SMAD2 Mothers against decapentaplegic | K.CVTIPSTC*SEIWGLSTPNTIDQWDTT | 1015 |
| C81 | homolog 2 | GLYSFSEQTR.S | |
| Q9NZB2_ | FAM120A Constitutive coactivator of | K.GSQMGTVQPIPC*LLSMPTR.N | 1016 |
| C531 | PPAR-gamma-like protein | | |
| Q9HB90_ | RRAGC Ras-related GTP-binding | R.SC*GHQTSASSLK.A | 1017 |
| C377 | protein C | | |
| Q9BR61_ | ACBD6 Acyl-CoA-binding domain- | R.DQDGCLPEEVTGC*K.T | 1018 |
| C267 | containing protein 6 | | |
| P16455_ | MGMT Methylated-DNA--protein-cysteine | R.GNPVPILIPC*HR.V | 1019 |
| C145 | methyltransferase | | |
| Q86UV5_ | USP48 Ubiquitin carboxyl-terminal | R.IWLEPC*IR.G | 1020 |
| C39 | hydrolase 48 | | |
| A2A288_ | ZC3H12D Probable ribonuclease | R.LAFSDDLGPLGPPLPVPAC*SLTPR.L | 1021 |
| C367 | ZC3H12D | | |
| Q8NEC7_ | GSTCD Glutathione S-transferase C- | K.AC*AEVSQWTR.L | 1022 |
| C140 | terminal domain-containing | | |
| Q6PJG6_ | BRAT1 BRCA1-associated ATM activator | R.THC*PYAVALPEVAPAQPLTEALR.A | 1023 |
| C673 | 1 | | |
| Q13232_ | NME3 Nucleoside diphosphate kinase 3 | R.ADELLC*WEDSAGHWLYE | 1024 |
| C158 | | | |
| Q86X76_ | NIT1 Nitrilase homolog 1 | K.THLC*DVEIPGQGPMCESNSTMPGPS | 1025 |
| C165 | | LESPVSTPAGK.I | |
| P42695_ | NCAPD3 Condensin-2 complex subunit | R.C*VMAMLR.R | 1026 |
| C541 | D3 | | |
| P41226_ | UBA7 Ubiquitin-like modifier- | R.APASAAASEDAPYPVC*TVR.Y | 1027 |
| C599 | activating enzyme 7 | | |
| Q99986_ | VRK1 Serine/threonine-protein kinase | K.VGLPIGQGGFGC*IYLADMNSSESVG | 1028 |
| C50 | VRK1 | SDAPCVVK.V | |
| Q8WUM4_ | PDCD61P Programmed cell death 6- | K.FPFSENQIC*LTFTWK.D | 1029 |
| C90 | interacting protein | | |
| P29590_ | PML Protein PML | R.GCSKPLCC*SCALLDSSHSELK.0 | 1030 |
| C213 | | | |
| Q9P0K7_ | RAI14 Ankycorbin | K.QILTMC*K.N | 1031 |
| C973 | | | |
| P53992_ | SEC24C Protein sport protein Sec24C | R.APPSSGAPPASTAQAPC*GQAAYGQF | 1032 |
| C78 | | GQGDVQNGPSSTVQMQR.L | |
| Q13867_ | BLMH Bleomycin hydrolase | R.C*WIFSCLNVMR.L | 1033 |
| C73 | | | |
| Q8ND24_ | RNF214 RING finger protein 214 | R.SSHAPATC*K.L | 1034 |
| C655 | | | |
| Q96EK4_ | THAP11 THAP domain-containing | R.AGVSGC*FSTFQPTTGHR.L | 1035 |
| C48 | protein 11 | | |
| Q961V0_ | NGLY1 Peptide-N(4)-(N-acetyl- | R.C*GEWANCFTLCCR.A | 1036 |
| C309 | beta-glucosaminypasparagin | | |
| Q5T1V6_ | DDX59 Probable ATP-dependent RNA | DDX59 K.NLPC*ANVR.Q | 1037 |
| C414 | helicase DDX59 | | |
| Q9UHQ1_ | NARF Nuclear prelamin A recognition | K.VLVVSVC*PQSLPYFAAK.F | 1038 |
| C99 | factor | | |
| O43396_ | TXNL1 Thioredoxin-like protein 1 | R.GC*GPCLR.I | 1039 |
| C34 | | | |
| Q81V53_ | DENND1C DENN domain-containing | R.GNSKPLSC*FVAPDSGRLPS1PENR.N | 1040 |
| C174 | protein IC | | |
| Q8N9T8_ | ICRI1 Protein ICRI1 homolog | R.LLGPTVMLGGC*EFSR.Q | 1041 |
| C673 | | | |

Table 3 illustrates a list of unliganded cysteines. Table 3 further shows the accession number (or the protein identifier) of the protein, cysteine residue number, and the respective SEQ ID NO.

TABLE 3

| | | SEQ |
|---|---|---|
| | | ID |
| Identifier | Protein Name | NO: |
| P26641_C266 | EEF1G Elongation factor 1- | 1042 |
| | gamma | |
| P07900_C374 | HSP90AA1 Heat shock protein | 1043 |
| | HSP 90-alpha | |
| P23528_C139 | CFL1 Cofilin-1 | 1044 |
| P68104_C411 | EEF1A1 Elongation factor 1- | 1045 |
| | alpha 1 | |
| P08238_C366 | HSP90AB1 Heat shock protein | 1046 |
| | HSP 90-beta | |
| P60981_C163 | DSTN Destrin | 1047 |
| P61978_C132 | HNRNPK Heterogeneous | 1048 |
| | nuclear ribonucleoprotein K | |
| P60709_C217 | ACTB Actin, cytoplasmic 1 | 1049 |
| Q15365_C54 | PCBP1 Poly(rC)-binding | 1050 |
| | protein 1 | |
| P60709_C272 | ACTB Actin, cytoplasmic 1 | 1051 |
| P27348_C134 | YWHAQ 14-3-3 protein theta | 1052 |
| P55769_C30 | NHP2L1 NHP2-like protein 1 | 1053 |
| Q6IS14_C129 | EIF5AL1 Eukaryotic | 1054 |
| | translation initiation factor 5A- | |
| | 1-like | |
| P11413_C13 | G6PD Glucose-6-phosphate 1- | 1055 |
| | dehydrogenase | |
| P04406_C247 | GAPDH Glyceraldehyde-3- | 1056 |
| | phosphate dehydrogenase | |
| P37802_C124 | TAGLN2 Transgelin-2 | 1057 |
| Q13185_C177 | CBX3 Chromobox protein | 1058 |
| | homolog 3 | |
| Q9Y3F4_C340 | STRAP Serine-threonine | 1059 |
| | kinase receptor-associated | |
| | protein | |
| Q9NTK5_C55 | OLA1 Obg-like ATPase 1 | 1060 |
| P53396_C20 | ACLY ATP-citrate synthase | 1061 |
| P62826_C112 | RAN GTP-binding nuclear | 1062 |
| | protein Ran | |
| P15880_C229 | RPS2 40S ribosomal protein | 1063 |
| | S2 | |
| P08238_C589 | HSP90AB1 Heat shock protein | 1064 |
| | HSP 90-beta | |
| P30084_C62 | ECHS1 Enoyl-CoA hydratase, | 1065 |
| | mitochondrial | |
| P47756_C36 | CAPZB F-actin-capping | 1066 |
| | protein subunit beta | |
| P52292_C223 | KPNA2 Importin subunit | 1067 |
| | alpha-2 | |
| Q15366_C109 | PCBP2 Poly(rC)-binding | 1068 |
| | protein 2 | |
| P14174_C81 | MIF Macrophage migration | 1069 |
| | inhibitory factor | |
| P61981_C97 | YWHAG 14-3-3 protein | 1070 |
| | gamma | |
| P00338_C163 | LDHA L-lactate | 1071 |
| | dehydrogenase A chain | |
| Q15019_C111 | SEPT2 Septin-2 | 1072 |
| P84243_C111 | H3F3B Histone H3.3 | 1073 |
| P18085_C62 | ARF4 ADP-ribosylation factor | 1074 |
| | 4 | |
| P10599_C73 | TXN Thioredoxin | 1075 |
| P13639_C466 | EEF2 Elongation factor 2 | 1076 |
| P00338_C293 | LDHA L-lactate | 1077 |
| | dehydrogenase A chain | |
| P49721_C91 | PSMB2 Proteasome subunit | 1078 |
| | beta type-2 | |
| P28062_C160 | PSMB8 Proteasome subunit | 1079 |
| | beta type-8 | |
| P10809_C237 | HSPD1 60 kDa heat shock | 1080 |
| | protein, mitochondrial | |
| P60981_C23 | DSTN Destrin | 1081 |
| P31943_C267 | HNRNPH1 Heterogeneous | 1082 |
| | nuclear ribonucleoprotein H | |
| P49321_C708 | NASP Nuclear autoantigenic | 1083 |
| | sperm protein | |
| Q99829_C53 | CPNE1 Copine-1 | 1084 |
| P68104_C111 | EEF1A1 Elongation factor 1- | 1085 |
| | alpha 1 | |
| Q02750_C207 | MAP2K1 Dual specificity | 1086 |
| | mitogen-activated protein | |
| | kinase | |
| O14950_C109 | MYL12B Myosin regulatory | 1087 |
| | light chain 12B | |
| P47756_C206 | CAPZB F-actin-capping | 1088 |
| | protein subunit beta | |
| P49327_C1448 | FASN Fatty acid synthase | 1089 |
| P14868_C349 | DARS Aspartate--tRNA | 1090 |
| | ligase, cytoplasmic | |
| P07437_C201 | TUBB Tubulin beta chain | 1091 |
| P07437_C303 | TUBB Tubulin beta chain | 1092 |
| P61077_C111 | UBE2D3 Ubiquitin- | 1093 |
| | conjugating enzyme E2 D3 | |
| P15153_C178 | RAC2 Ras-related C3 | 1094 |
| | botulinum toxin substrate 2 | |
| Q04637_C662 | EIF4G1 Eukaryotic translation | 1095 |
| | initiation factor 4 gamma 1 | |
| P78347_C215 | GTF2I General transcription | 1096 |
| | factor II-I | |
| Q15370_C89 | TCEB2 Transcription | 1097 |
| | elongation factor B | |
| | polypeptide 2 | |
| P62753_C12 | RPS6 40S ribosomal protein | 1098 |
| | S6 | |
| P31948_C461 | STIP1 Stress-induced- | 1099 |
| | phosphoprotein 1 | |
| Q9UL46_C91 | PSME2 Proteasome activator | 1100 |
| | complex subunit 2 | |
| P63220_C56 | RPS21 40S ribosomal protein | 1101 |
| | S21 | |
| P55809_C67 | OXCT1 Succinyl-CoA: 3- | 1102 |
| | ketoacid coenzyme A | |
| | transferase 1, | |
| P30153_C377 | PPP2R1A Serine/threonine- | 1103 |
| | protein phosphatase 2A 65 kDa | |
| | regulatory subunit A alpha | |
| | isoform | |
| P30153_C390 | PPP2R1A Serine/threonine- | 1104 |
| | protein phosphatase 2A 65 kDa | |
| | regulatory subunit A alpha | |
| | isoform | |
| P60866_C70 | RPS20 40S ribosomal protein | 1105 |
| | S20 | |
| Q9P258_C428 | RCC2 Protein RCC2 | 1106 |
| P60709_C285 | ACTB Actin, cytoplasmic 1 | 1107 |
| P22234_C81 | PAICS Multifunctional protein | 1108 |
| | ADE2 | |
| P27348_C237 | YWHAQ 14-3-3 protein theta | 1109 |
| P12004_C81 | PCNA Proliferating cell | 1110 |
| | nuclear antigen | |
| P68104_C234 | EEF1A1 Elongation factor 1- | 1111 |
| | alpha 1 | |
| Q96FW1_C23 | OTUB1 Ubiquitin thioesterase | 1112 |
| | OTUB1 | |
| Q13200_C779 | PSMD2 26S proteasome non- | 1113 |
| | ATPase regulatory subunit 2 | |
| P22314_C632 | UBA1 Ubiquitin-like modifier- | 1114 |
| | activating enzyme 1 | |
| P37802_C38 | TAGLN2 Transgelin-2 | 1115 |
| P37802_C63 | TAGLN2 Transgelin-2 | 1116 |
| P46782_C172 | RPS5 40S ribosomal protein | 1117 |
| | S5 | |
| P04075_C339 | ALDOA Fructose- | 1118 |
| | bisphosphate aldolase A | |
| P05386_C61 | RPLP1 60S acidic ribosomal | 1119 |
| | protein P1 | |
| P63000_C178 | RAC1 Ras-related C3 | 1120 |
| | botulinum toxin substrate 1 | |
| P07195_C294 | LDHB L-lactate | 1121 |
| | dehydrogenase B chain | |
| Q13148_C244 | TARDBP TAR DNA-binding | 1122 |
| | protein 43 | |
| P30048_C229 | PRDX3 Thioredoxin- | 1123 |
| | dependent peroxide reductase, | |
| | mitochondrial | |
| P62306_C66 | SNRPF Small nuclear | 1124 |
| | ribonucleoprotein F | |
| O15371_C19 | EIF3D Eukaryotic translation | 1125 |
| | initiation factor 3 subunit | |
| P17987_C397 | TCP1 T-complex protein 1 | 1126 |
| | subunit alpha | |
| Q9Y5P6_C245 | GMPPB Mannose-1-phosphate | 1127 |
| | guanyltransferase beta | |
| Q9BW61_C25 | DDA1 DET1- and DDB1- | 1128 |
| | associated protein 1 | |
| Q14974_C585 | KPNB1 Importin subunit beta- | 1129 |
| | 1 | |
| Q9BUF5_C303 | TUBB6 Tubulin beta-6 chain | 1130 |
| P52272_C653 | HNRNPM Heterogeneous | 1131 |
| | nuclear ribonucleoprotein M | |
| O75940_C214 | SMNDC1 Survival of motor | 1132 |
| | neuron-related-splicing factor | |
| | 3 | |
| O00233_C59 | PSMD9 26S proteasome non- | 1133 |
| | ATPase regulatory subunit 9 | |
| P63208_C160 | SKP1 S-phase kinase- | 1134 |
| | associated protein 1 | |
| Q9Y224_C19 | C14orf166 UPF0568 protein | 1135 |
| | C14orf166 | |
| P30626_C194 | SRI Sorcin | 1136 |
| Q9BXW7_C392 | CECR5 Cat eye syndrome | 1137 |
| | critical region protein 5 | |
| P60842_C134 | EIF4A1 Eukaryotic initiation | 1138 |
| | factor 4A-I | |
| P63220_C17 | RPS21 40S ribosomal protein | 1139 |
| | S21 | |
| P50990_C149 | CCT8 T-complex protein 1 | 1140 |
| | subunit theta | |
| P22234_C63 | PAICS Multifunctional protein | 1141 |
| | ADE2 | |
| Q13148_C39 | TARDBP TAR DNA-binding | 1142 |
| | protein 43 | |
| P17987_C357 | TCP1 T-complex protein 1 | 1143 |
| | subunit alpha | |
| P31946_C96 | YWHAB 14-3-3 protein | 1144 |
| | beta/alpha | |
| P61289_C92 | PSME3 Proteasome activator | 1145 |
| | complex subunit 3 | |
| P26641_C68 | EEF1G Elongation factor 1- | 1146 |
| | gamma | |
| P78371_C395 | CCT2 T-complex protein 1 | 1147 |
| | subunit beta | |
| P14868_C203 | DARS Aspartate--tRNA | 1148 |
| | ligase, cytoplasmic | |
| Q92530_C185 | PSMF1 Proteasome inhibitor | 1149 |
| | PI31 subunit | |
| P25398_C92 | RPS12 40S ribosomal protein | 1150 |
| | S12 | |
| P25205_C148 | MCM3 DNA replication | 1151 |
| | licensing factor MCM3 | |
| P60174_C79 | TPI1 Triosephosphate | 1152 |
| | isomerase | |
| P43487_C132 | RANBP1 Ran-specific | 1153 |
| | GTPase-activating protein | |
| P62280_C131 | RPS11 40S ribosomal protein | 1154 |
| | S11 | |
| P13639_C651 | EEF2 Elongation factor 2 | 1155 |
| P13639_C751 | EEF2 Elongation factor 2 | 1156 |
| Q9NUQ9_C10 | FAM49B Protein FAM49B | 1157 |
| P62937_C62 | PPIA Peptidyl-prolyl cis-trans | 1158 |
| | isomerase A | |
| P23528_C39 | CFL1 Cofilin-1 | 1159 |
| P46776_C70 | RPL27A 60S ribosomal | 1160 |
| | protein L27a | |
| P49915_C456 | GMPS GMP synthase | 1161 |
| Q16851_C123 | UGP2 UTP-glucose-1- | 1162 |
| | phosphate uridylyltransferase | |
| P14618_C358 | PKM Pyruvate kinase | 1163 |
| | isozymes M1/M2 | |
| P14618_C474 | PKM Pyruvate kinase | 1164 |
| | isozymes M1/M2 | |
| P13489_C409 | RNH1 Ribonuclease inhibitor | 1165 |
| O15355_C241 | PPM1G Protein phosphatase | 1166 |
| | 1G | |
| P68431_C111 | HIST1H3J Histone H3.1 | 1167 |
| P08238_C521 | HSP90AB1 Heat shock protein | 1168 |
| | HSP 90-beta | |
| Q99439_C175 | CNN2 Calponin-2 | 1169 |
| O00299_C223 | CLIC1 Chloride intracellular | 1170 |
| | channel protein 1 | |
| Q96KB5_C22 | PBK Lymphokine-activated | 1171 |
| | killer T-cell-originated protein | |
| | kinase | |
| P00492_C106 | HPRT1 Hypoxanthine-guanine | 1172 |
| | phosphoribosyltransferase | |
| Q15631_C225 | TSN Translin | 1173 |
| P52565_C79 | ARHGDIA Rho GDP- | 1174 |
| | dissociation inhibitor 1 | |
| P78417_C237 | GSTO1 Glutathione S- | 1175 |
| | transferase omega-1 | |
| Q01518_C93 | CAP1 Adenylyl cyclase- | 1176 |
| | associated protein 1 | |
| Q13148_C50 | TARDBP TAR DNA-binding | 1177 |
| | protein 43 | |
| P51665_C116 | PSMD7 26S proteasome non- | 1178 |
| | ATPase regulatory subunit 7 | |
| P17987_C147 | TCP1 T-complex protein 1 | 1179 |
| | subunit alpha | |
| O00299_C59 | CLIC1 Chloride intracellular | 1180 |
| | channel protein 1 | |
| P61077_C85 | UBE2D3 Ubiquitin- | 1181 |
| | conjugating enzyme E2 D3 | |
| Q06830_C173 | PRDX1 Peroxiredoxin-1 | 1182 |
| P06733_C389 | ENO1 Alpha-enolase | 1183 |
| P28062_C120 | PSMB8 Proteasome subunit | 1184 |
| | beta type-8 | |
| Q96B23_C57 | C18orf25 Uncharacterized | 1185 |
| | protein C18orf25 | |
| P05388_C119 | RPLP0 60S acidic ribosomal | 1186 |
| | protein P0 | |
| Q14353_C91 | GAMT Guanidinoacetate N- | 1187 |
| | methyltransferase | |
| P11142_C17 | HSPA8 Heat shock cognate 71 | 1188 |
| | kDa protein | |
| Q9UI30_C33 | TRMT112 tRNA | 1189 |
| | methyltransferase 112 | |
| | homolog | |
| P27348_C94 | YWHAQ 14-3-3 protein theta | 1190 |
| P51812_C579 | RPS6KA3 Ribosomal protein | 1191 |
| | S6 kinase alpha-3 | |
| P62304_C46 | SNRPE Small nuclear | 1192 |
| | ribonucleoprotein E | |
| P67936_C247 | TPM4 Tropomyosin alpha-4 | 1193 |
| | chain | |
| Q9H3P7_C129 | ACBD3 Golgi resident protein | 1194 |
| | GCP60 | |
| P49589_C27 | CARS Cysteine--tRNA ligase, | 1195 |
| | cytoplasmic | |
| Q9BY32_C146 | ITPA Inosine triphosphate | 1196 |
| | pyrophosphatase | |
| P25205_C360 | MCM3 DNA replication | 1197 |
| | licensing factor MCM3 | |
| Q96GX9_C147 | APIP Probable | 1198 |
| | methylthioribulose-1- | |
| | phosphate dehydratas | |
| P18669_C153 | PGAM1 Phosphoglycerate | 1199 |
| | mutase 1 | |
| O75390_C211 | CS Citrate synthase, | 1200 |
| | mitochondrial | |
| Q16555_C504 | DPYSL2 | 1201 |
| | Dihydropyrimidinase-related | |
| | protein 2 | |
| P62993_C32 | GRB2 Growth factor receptor- | 1202 |
| | bound protein 2 | |
| P63104_C94 | YWHAZ 14-3-3 protein | 1203 |
| | zeta/delta | |
| B0V043_C112 | VARS Valyl-tRNA synthetase | 1204 |
| P62937_C161 | PPIA Peptidyl-prolyl cis-trans | 1205 |
| | isomerase A | |
| Q8N806_C35 | UBR7 Putative E3 ubiquitin- | 1206 |
| | protein ligase UBR7 | |
| P10768_C56 | ESD S-formylglutathione | 1207 |
| | hydrolase | |
| Q16630_C159 | CPSF6 Cleavage and | 1208 |
| | polyadenylation specificity | |
| | factor subunit | |
| P47756_C147 | CAPZB F-actin-capping | 1209 |
| | protein subunit beta | |
| Q9UMR2_C393 | DDX19B ATP-dependent | 1210 |
| | RNA helicase DDX19B | |
| P00558_C379 | PGK1 Phosphoglycerate | 1211 |
| | kinase 1 | |
| Q9NZZ3_C20 | CHMP5 Charged | 1212 |
| | multivesicular body protein 5 | |
| P51610_C1886 | HCFC1 Host cell factor 1 | 1213 |
| P41250_C471 | GARS Glycine-tRNA ligase | 1214 |
| O00299_C191 | CLIC1 Chloride intracellular | 1215 |
| | channel protein 1 | |
| Q9BV86_C195 | NTMT1 N-terminal Xaa-Pro- | 1216 |
| | Lys N-methyltransferase 1 | |
| Q15369_C112 | TCEB1 Transcription | 1217 |
| | elongation factor B | |
| | polypeptide 1 | |
| P14618_C49 | PKM Pyruvate kinase | 1218 |
| | isozymes M1/M2 | |
| O43707_C499 | ACTN4 Alpha-actinin-4 | 1219 |
| Q16576_C97 | RBBP7 Histone-binding | 1220 |
| | protein RBBP7 | |
| P28838_C462 | LAP3 Cytosol aminopeptidase | 1221 |
| Q00839_C607 | HNRNPU Heterogeneous | 1222 |
| | nuclear ribonucleoprotein U | |
| O95861_C42 | BPNT1 3(2),5-bisphosphate | 1223 |
| | nucleotidase 1 | |
| P40926_C212 | MDH2 Malate dehydrogenase, | 1224 |
| | mitochondrial | |
| Q9Y2Z0_C62 | SUGT1 Suppressor of G2 | 1225 |
| | allele of SKP1 homolog | |
| O60828_C60 | PQBP1 Polyglutamine-binding | 1226 |
| | protein 1 | |
| P09661_C89 | SNRPA1 U2 small nuclear | 1227 |
| | ribonucleoprotein A | |
| P63279_C138 | UBE2I SUMO-conjugating | 1228 |
| | enzyme UBC9 | |
| P62258_C98 | YWHAE 14-3-3 protein | 1229 |
| | epsilon | |
| Q8WVJ2_C99 | NUDCD2 NudC domain- | 1230 |
| | containing protein 2 | |
| Q15365_C158 | PCBP1 Poly(rC)-binding | 1231 |
| | protein 1 | |
| P07900_C597 | HSP90AA1 Heat shock protein | 1232 |
| | HSP 90-alpha | |
| P21333_C1157 | FLNA Filamin-A | 1233 |
| P14618_C326 | PKM Pyruvate kinase | 1234 |
| | isozymes M1/M2 | |
| Q13363_C134 | CTBP1 C-terminal-binding | 1235 |
| | protein 1 | |
| P48643_C181 | CCT5 T-complex protein 1 | 1236 |
| | subunit epsilon | |
| P00338_C35 | LDHA L-lactate | 1237 |
| | dehydrogenase A chain | |
| P25789_C107 | PSMA4 Proteasome subunit | 1238 |
| | alpha type-4 | |
| P28838_C335 | LAP3 Cytosol aminopeptidase | 1239 |
| P00558_C316 | PGK1 Phosphoglycerate | 1240 |
| | kinase 1 | |
| Q9BQ69_C186 | MACROD1 O-acetyl-ADP- | 1241 |
| | ribose deacetylase MACROD1 | |
| P23368_C120 | ME2 NAD-dependent malic | 1242 |
| | enzyme, mitochondrial | |
| P07195_C36 | LDHB L-lactate | 1243 |
| | dehydrogenase B chain | |
| Q92879_C150 | CELF1 CUGBP Elav-like | 1244 |
| | family member 1 | |
| Q9Y696_C100 | CLIC4 Chloride intracellular | 1245 |
| | channel protein 4 | |
| Q8NBF2_C716 | NHLRC2 NHL repeat- | 1246 |
| | containing protein 2 | |
| P24534_C50 | EEF1B2 Elongation factor 1- | 1247 |
| | beta | |
| P46782_C155 | RPS5 40S ribosomal protein | 1248 |
| | S5 | |
| H3BQZ7_C57 | Uncharacterized protein | 1249 |
| Q93052_C364 | LPP Lipoma-preferred partner | 1250 |
| P04075_C202 | ALDOA Fructose- | 1251 |
| | bisphosphate aldolase A | |
| Q7KZF4_C560 | SND1 Staphylococcal nuclease | 1252 |
| | domain-containing protein | |
| P40227_C406 | CCT6A T-complex protein 1 | 1253 |
| | subunit zeta | |
| P60174_C255 | TPI1 Triosephosphate | 1254 |
| | isomerase | |
| Q08J23_C321 | NSUN2 tRNA (cytosine(34)- | 1255 |
| | C(5))-methyltransferase | |
| P15927_C219 | RPA2 Replication protein A 32 | 1256 |
| | kDa subunit | |
| P09622_C477 | DLD Dihydrolipoyl | 1257 |
| | dehydrogenase, mitochondrial | |
| P28838_C376 | LAP3 Cytosol aminopeptidase | 1258 |
| P49915_C523 | GMPS GMP synthase | 1259 |
| Q9H3H3_C222 | C11orf68 UPF0696 protein | 1260 |
| | C11orf68 | |
| P07737_C128 | PFN1 Profilin-1 | 1261 |
| Q00839_C594 | HNRNPU Heterogeneous | 1262 |
| | nuclear ribonucleoprotein U | |
| Q14974_C455 | KPNB1 Importin subunit beta- | 1263 |
| | 1 | |
| O15067_C66 | PFAS | 1264 |
| | Phosphoribosylformylglycinamidine | |
| | synthase | |
| Q01813_C112 | PFKP 6-phosphofructokinase | 1265 |
| | type C | |
| P11413_C385 | G6PD Glucose-6-phosphate 1- | 1266 |
| | dehydrogenase | |
| Q13561_C240 | DCTN2 Dynactin subunit 2 | 1267 |
| Q13162_C245 | PRDX4 Peroxiredoxin-4 | 1268 |
| P50991_C252 | CCT4 T-complex protein 1 | 1269 |
| | subunit delta | |
| P04899_C112 | GNAI2 Guanine nucleotide- | 1270 |
| | binding protein G(i) subunit | |
| | alpha I2 | |
| O43765_C153 | SGTA Small glutamine-rich | 1271 |
| | tetratricopeptide repeat- | |
| | containing protein alpha | |
| P34932_C290 | HSPA4 Heat shock 70 kDa | 1272 |
| | protein 4 | |
| P61247_C96 | RPS3A 40S ribosomal protein | 1273 |
| | S3a | |
| P04075_C73 | ALDOA Fructose- | 1274 |
| | bisphosphate aldolase A | |
| Q96EP5_C85 | DAZAP1 DAZ-associated | 1275 |
| | protein 1 | |
| P27708_C1889 | CAD CAD protein | 1276 |
| Q16822_C431 | PCK2 Phosphoenolpyruvate | 1277 |
| | carboxykinase | |
| Q96KB5_C70 | PBK Lymphokine-activated | 1278 |
| | killer T-cell-originated protein | |
| P00558_C108 | PGK1 Phosphoglycerate | 1279 |
| | kinase 1 | |
| Q15365_C293 | PCBP1 Poly(rC)-binding | 1280 |
| | protein 1 | |
| P60174_C164 | TPI1 Triosephosphate | 1281 |
| | isomerase | |
| P62826_C120 | RAN GTP-binding nuclear | 1282 |
| | protein Ran | |
| O75348_C69 | ATP6V1G1 V-type proton | 1283 |
| | ATPase subunit G 1 | |
| Q9Y2D5_C296 | AKAP2 A-kinase anchor | 1284 |
| | protein 2 | |
| P61158_C12 | ACTR3 Actin-related protein 3 | 1285 |
| P36405_C118 | ARL3 ADP-ribosylation | 1286 |
| | factor-like protein 3 | |
| P62937_C115 | PPIA Peptidyl-prolyl cis-trans | 1287 |
| | isomerase A | |
| P35754_C83 | GLRX Glutaredoxin-1 | 1288 |
| O60664_C341 | PLIN3 Perilipin-3 | 1289 |
| Q12874_C103 | SF3A3 Splicing factor 3A | 1290 |
| | subunit 3 | |
| Q9H4M9_C138 | EHD1 EH domain-containing | 1291 |
| | protein 1 | |
| Q15185_C76 | PTGES3 Prostaglandin E | 1292 |
| | synthase 3 | |
| P04632_C190 | CAPNS1 Calpain small | 1293 |
| | subunit 1 | |
| P13489_C75 | RNH1 Ribonuclease inhibitor | 1294 |
| O95372_C213 | LYPLA2 Acyl-protein | 1295 |
| | thioesterase 2 | |
| P84085_C62 | ARF5 ADP-ribosylation factor | 1296 |
| | 5 | |
| P55786_C265 | NPEPPS Puromycin-sensitive | 1297 |
| | aminopeptidase | |
| Q14204_C1977 | DYNC1H1 Cytoplasmic | 1298 |
| | dynein 1 heavy chain 1 | |
| P14866_C581 | HNRNPL Heterogeneous | 1299 |
| | nuclear ribonucleoprotein L | |
| Q14980_C160 | NUMA1 Nuclear mitotic | 1300 |
| | apparatus protein 1 | |
| O00743_C192 | PPP6C Serine/threonine- | 1301 |
| | protein phosphatase 6 catalytic | |
| | subunit | |
| P07741_C83 | APRT Adenine | 1302 |
| | phosphoribosyltransferase | |
| O75607_C105 | NPM3 Nucleoplasmin-3 | 1303 |
| P09429_C23 | HMGB1 High mobility group | 1304 |
| | protein B1 | |
| P26641_C339 | EEF1G Elongation factor 1- | 1305 |
| | gamma | |
| O60232_C152 | SSSCA1 Sjoegren | 1306 |
| | syndrome/scleroderma | |
| | autoantigen 1 | |
| Q66K74_C342 | MAP1S Microtubule- | 1307 |
| | associated protein 1S | |
| Q52LJ0_C63 | FAM98B Protein FAM98B | 1308 |
| P62249_C25 | RPS16 40S ribosomal protein | 1309 |
| | S16 | |
| P35579_C988 | MYH9 Myosin-9 | 1310 |
| P33316_C222 | DUT Deoxyuridine 5- | 1311 |
| | triphosphate | |
| | nucleotidohydrolase, | |
| Q9Y6E0_C89 | STK24 Serine/threonine- | 1312 |
| | protein kinase 24 | |
| P49411_C222 | TUFM Elongation factor Tu, | 1313 |
| | mitochondrial | |
| Q01813_C360 | PFKP 6-phosphofructokinase | 1314 |
| | type C | |
| Q7L2J0_C177 | MEPCE 7SK snRNA | 1315 |
| | methylphosphate capping | |
| | enzyme | |
| O15355_C164 | PPM1G Protein phosphatase | 1316 |
| | 1G | |
| Q6IBS0_C141 | TWF2 Twinfilin-2 | 1317 |
| Q9NVG8_C387 | TBC1D13 TBC1 domain | 1318 |
| | family member 13 | |
| O15382_C342 | BCAT2 Branched-chain- | 1319 |
| | amino-acid aminotransferase, | |
| | mitochandrial | |
| P12004_C135 | PCNA Proliferating cell | 1320 |
| | nuclear antigen | |
| P60660_C32 | MYL6 Myosin light | 1321 |
| | polypeptide 6 | |
| P55072_C522 | VCP Transitional endoplasmic | 1322 |
| | reticulum ATPase | |
| Q99439_C240 | CNN2 Calponin-2 | 1323 |
| P05455_C245 | SSB Lupus La protein | 1324 |
| O75131_C54 | CPNE3 Copine-3 | 1325 |
| P26358_C62 | DNMT1 DNA (cytosine-5)- | 1326 |
| | methyltransferase 1 | |
| Q7Z4W1_C138 | DCXR L-xylulose reductase | 1327 |
| P10809_C447 | HSPD1 60 kDa heat shock | 1328 |
| | protein, mitochondrial | |
| P84077_C159 | ARF1 ADP-ribosylation factor | 1329 |
| | 1 | |
| Q15185_C40 | PTGES3 Prostaglandin E | 1330 |
| | synthase 3 | |
| Q16854_C87 | DGUOK Deoxyguanosine | 1331 |
| | kinase, mitochondrial | |
| Q9Y508_C8 | RNF114 RING finger protein | 1332 |
| | 114 | |
| P63208_C120 | SKP1 S-phase kinase- | 1333 |
| | associated protein 1 | |
| O00148_C164 | DDX39A ATP-dependent | 1334 |
| | RNA helicase DDX39A | |
| P25786_C148 | PSMA1 Proteasome subunit | 1335 |
| | alpha type-1 | |
| P61221_C88 | ABCE1 ATP-binding cassette | 1336 |
| | sub-family E member 1 | |
| O60664_C60 | PLIN3 Perilipin-3 | 1337 |
| P15311_C117 | EZR Ezrin | 1338 |
| Q9UBW8_C110 | COPS7A COP9 signalosome | 1339 |
| | complex subunit 7a | |
| P42765_C287 | ACAA2 3-ketoacyl-CoA | 1340 |
| | thiolase, mitochondrial | |
| Q99832_C158 | CCT7 T-complex protein 1 | 1341 |
| | subunit eta | |
| P61978_C145 | HNRNPK Heterogeneous | 1342 |
| | nuclear ribonucleoprotein K | |
| Q13310_C132 | PABPC4 Polyadenylate- | 1343 |
| | binding protein 4 | |
| P35998_C377 | PSMC2 26S protease | 1344 |
| | regulatory subunit 7 | |
| P52789_C794 | HK2 Hexokinase-2 | 1345 |
| P35579_C91 | MYH9 Myosin-9 | 1346 |
| P13995_C166 | MTHFD2 Bifunctional | 1347 |
| | methylenetetrahydrofolate | |
| | dehydrogena | |
| P22102_C41 | GART Trifunctional purine | 1348 |
| | biosynthetic protein adenosin | |
| P0CW22_C35 | RPS17L 40S ribosomal protein | 1349 |
| | S17-like | |
| Q9UK45_C85 | LSM7 U6 snRNA-associated | 1350 |
| | Sm-like protein LSm7 | |
| Q12874_C274 | SF3A3 Splicing factor 3A | 1351 |
| | subunit 3 | |
| P34897_C119 | SHMT2 Serine | 1352 |
| | hydroxymethyltransferase, | |
| | mitochondrial | |
| P15153_C157 | RAC2 Ras-related C3 | 1353 |
| | botulinum toxin substrate 2 | |
| Q16186_C88 | ADRM1 Proteasomal ubiquitin | 1354 |
| | receptor ADRM1 | |
| P15531_C145 | NME1 Nucleoside diphosphate | 1355 |
| | kinase A | |
| P49327_C2202 | FASN Fatty acid synthase | 1356 |
| P40926_C275 | MDH2 Malate dehydrogenase, | 1357 |
| | mitochondrial | |
| P22314_C23 | UBA1 Ubiquitin-like modifier- | 1358 |
| | activating enzyme 1 | |
| P52789_C517 | HK2 Hexokinase-2 | 1359 |
| Q12931_C573 | TRAP1 Heat shock protein 75 | 1360 |
| | kDa, mitochondrial | |
| P39687_C123 | ANP32A Acidic leucine-rich | 1361 |
| | nuclear phosphoprotein 32 | |
| | fami | |
| O43865_C272 | AHCYL1 Putative | 1362 |
| | adenosylhomocysteinase 2 | |
| O95456_C157 | PSMG1 Proteasome assembly | 1363 |
| | chaperone 1 | |
| P49005_C384 | POLD2 DNA polymerase delta | 1364 |
| | subunit 2 | |
| Q96EY8_C132 | MMAB Cob(I)yrinic acid a,c- | 1365 |
| | diamide adenosyltransferase, | |
| P28074_C111 | PSMB5 Proteasome subunit | 1366 |
| | beta type-5 | |
| O75446_C184 | SAP30 Histone deacetylase | 1367 |
| | complex subunit SAP30 | |
| P05388_C27 | RPLP0 60S acidic ribosomal | 1368 |
| | protein P0 | |
| Q15366_C158 | PCBP2 Poly(rC)-binding | 1369 |
| | protein 2 | |
| O75369_C2501 | FLNB Filamin-B | 1370 |
| Q06124_C259 | PTPN11 Tyrosine-protein | 1371 |
| | phosphatase non-receptor type | |
| | 11 | |
| P40926_C285 | MDH2 Malate dehydrogenase, | 1372 |
| | mitochondrial | |
| Q9BV79_C263 | MECR Trans-2-enoyl-CoA | 1373 |
| | reductase, mitochondrial | |
| Q09666_C1900 | AHNAK Neuroblast | 1374 |
| | differentiation-associated | |
| | protein AHNA | |
| Q9UBB5_C359 | MBD2 Methyl-CpG-binding | 1375 |
| | domain protein 2 | |
| P32322_C262 | PYCR1 Pyrroline-5- | 1376 |
| | carboxylate reductase 1, | |
| | mitochondrial | |
| P31939_C241 | ATIC Bifunctional purine | 1377 |
| | biosynthesis protein PURH | |
| Q04917_C97 | YWHAH 14-3-3 protein eta | 1378 |
| P62753_C100 | RPS6 40S ribosomal protein | 1379 |
| | S6 | |
| Q9Y490_C1353 | TLN1 Talin-1 | 1380 |
| P52701_C615 | MSH6 DNA mismatch repair | 1381 |
| | protein Msh6 | |
| P49591_C300 | SARS Serine--tRNA ligase, | 1382 |
| | cytoplasmic | |
| P49411_C127 | TUFM Elongation factor Tu, | 1383 |
| | mitochondrial | |
| P51610_C227 | HCFC1 Host cell factor 1 | 1384 |
| P60174_C104 | TPI1 Triosephosphate | 1385 |
| | isomerase | |
| Q12982_C295 | BNIP2 BCL2/adenovirus E1B | 1386 |
| | 19 kDa protein-interacting pro | |
| Q9UHD8_C375 | SEPT9 Septin-9 | 1387 |
| P53618_C888 | COPB1 Coatomer subunit beta | 1388 |
| Q8N163_C644 | KIAA1967 DBIRD complex | 1389 |
| | subunit KIAA1967 | |
| P61106_C40 | RAB14 Ras-related protein | 1390 |
| | Rab-14 | |
| P21291_C167 | CSRP1 Cysteine and glycine- | 1391 |
| | rich protein 1 | |
| Q07020_C134 | RPL18 60S ribosomal protein | 1392 |
| | L18 | |
| Q86VP6_C413 | CAND1 Cullin-associated | 1393 |
| | NEDD8-dissociated protein 1 | |
| Q9Y3F4_C305 | STRAP Serine-threonine | 1394 |
| | kinase receptor-associated | |
| | protein | |
| Q9Y5K6_C540 | CD2AP CD2-associated | 1395 |
| | protein | |
| O60361_C130 | NME2P1 Putative nucleoside | 1396 |
| | diphosphate kinase | |
| Q9BWD1_C65 | ACAT2 Acetyl-CoA | 1397 |
| | acetyltransferase, cytosolic | |
| Q7L2J0_C522 | MEPCE 7SK snRNA | 1398 |
| | methylphosphate capping | |
| | enzyme | |
| P61158_C307 | ACTR3 Actin-related protein 3 | 1399 |
| P11142_C603 | HSPA8 Heat shock cognate 71 | 1400 |
| | kDa protein | |
| P13639_C728 | EEF2 Elongation factor 2 | 1401 |
| P04183_C206 | TK1 Thymidine kinase, | 1402 |
| | cytosolic | |
| P30153_C294 | PPP2R1A Serine/threonine- | 1403 |
| | protein phosphatase 2A 65 kDa | |
| | regulatory subunit A | |
| Q09666_C2162 | AHNAK Neuroblast | 1404 |
| | differentiation-associated | |
| | protein AHNA | |
| P46779_C13 | RPL28 60S ribosomal protein | 1405 |
| | L28 | |
| Q9Y696_C234 | CLIC4 Chloride intracellular | 1406 |
| | channel protein 4 | |
| P08670_C328 | VIM Vimentin | 1407 |
| O43684_C129 | BUB3 Mitotic checkpoint | 1408 |
| | protein BUB3 | |
| Q04917_C112 | YWHAH 14-3-3 protein eta | 1409 |
| P41252_C526 | IARS Isoleucine-tRNA ligase, | 1410 |
| | cytoplasmic | |
| O14980_C1070 | XPO1 Exportin-1 | 1411 |
| Q9Y3D5_C90 | MRPS18C 28S ribosomal | 1412 |
| | protein S18c, mitochondrial | |
| Q15459_C244 | SF3A1 Splicing factor 3A | 1413 |
| | subunit 1 | |
| P50552_C334 | VASP Vasodilator-stimulated | 1414 |
| | phosphoprotein | |
| Q04637_C1265 | EIF4G1 Eukaryotic translation | 1415 |
| | initiation factor 4 gamma 1 | |
| Q12824_C147 | SMARCB1 SWI/SNF-related | 1416 |
| | matrix-associated actin- | |
| | dependent | |
| P78346_C87 | RPP30 Ribonuclease P protein | 1417 |
| | subunit p30 | |
| Q99460_C806 | PSMD1 26S proteasome non- | 1418 |
| | ATPase regulatory subunit 1 | |
| P46060_C573 | RANGAP1 Ran GTPase- | 1419 |
| | activating protein 1 | |
| Q6P1X6_C98 | C8orf82 UPF0598 protein | 1420 |
| | C8orf82 | |
| P49189_C484 | ALDH9A1 4- | 1421 |
| | trimethylaminobutyraldehyde | |
| | dehydrogenase | |
| Q9NVG8_C36 | TBC1D13 TBC1 domain | 1422 |
| | family member 13 | |
| P49841_C199 | GSK3B Glycogen synthase | 1423 |
| | kinase-3 beta | |
| Q09666_C1833 | AHNAK Neuroblast | 1424 |
| | differentiation-associated | |
| | protein AHNA | |
| P62241_C182 | RPS8 40S ribosomal protein | 1425 |
| | S8 | |
| Q13620_C787 | CUL4B Cullin-4B | 1426 |
| P26599_C23 | PTBP1 Polypyrimidine tract- | 1427 |
| | binding protein 1 | |
| Q14697_C502 | GANAB Neutral alpha- | 1428 |
| | glucosidase AB | |
| P08238_C564 | HSP90AB1 Heat shock protein | 1429 |
| | HSP 90-beta | |
| P53582_C14 | METAP1 Methionine | 1430 |
| | aminopeptidase 1 | |
| Q9UBT2_C30 | UBA2 SUMO-activating | 1431 |
| | enzyme subunit 2 | |
| P06733_C119 | ENO1 Alpha-enolase | 1432 |
| P55884_C384 | EIF3B Eukaryotic translation | 1433 |
| | initiation factor 3 subunit | |
| P63167_C24 | DYNLL1 Dynein light chain 1, | 1434 |
| | cytoplasmic | |
| Q15417_C173 | CNN3 Calponin-3 | 1435 |
| P21980_C230 | TGM2 Protein-glutamine | 1436 |
| | gamma-glutamyltransferase 2 | |
| P31943_C122 | HNRNPH1 Heterogeneous | 1437 |
| | nuclear ribonucleoprotein H | |
| P31947_C38 | SFN 14-3-3 protein sigma | 1438 |
| P18085_C159 | ARF4 ADP-ribosylation factor | 1439 |
| | 4 | |
| P60174_C124 | TPI1 Triosephosphate | 1440 |
| | isomerase | |
| P14618_C152 | PKM Pyruvate kinase | 1441 |
| | isozymes M1/M2 | |
| Q12765_C324 | SCRN1 Secernin-1 | 1442 |
| Q16555_C179 | DPYSL2 | 1443 |
| | Dihydropyrimidinase-related | |
| | protein 2 | |
| P35579_C1379 | MYH9 Myosin-9 | 1444 |
| P07814_C92 | EPRS Bifunctional | 1445 |
| | glutamate/proline--tRNA | |
| | ligase | |
| Q92597_C168 | NDRG1 Protein NDRG1 | 1446 |
| O43707_C351 | ACTN4 Alpha-actinin-4 | 1447 |
| Q7RTV0_C49 | PHF5A PHD finger-like | 1448 |
| | domain-containing protein 5A | |
| P53396_C845 | ACLY ATP-citrate synthase | 1449 |
| P17844_C234 | DDX5 Probable ATP- | 1450 |
| | dependent RNA helicase | |
| | DDX5 | |
| Q9H0C8_C325 | ILKAP Integrin-linked kinase- | 1451 |
| | associated serine/threonine | |
| P11413_C446 | G6PD Glucose-6-phosphate 1- | 1452 |
| | dehydrogenase | |
| Q9BTT0_C87 | ANP32E Acidic leucine-rich | 1453 |
| | nuclear phosphoprotein 32 | |
| | family member # | |
| Q14258_C70 | TRIM25 E3 ubiquitin/ISG15 | 1454 |
| | ligase TRIM25 | |
| Q8WU79_C196 | SMAP2 Stromal membrane- | 1455 |
| | associated protein 2 | |
| Q92947_C289 | GCDH Glutaryl-CoA | 1456 |
| | dehydrogenase, mitochondrial | |
| P45974_C195 | USP5 Ubiquitin carboxyl- | 1457 |
| | terminal hydrolase 5 | |
| Q9BXJ9_C817 | NAA15 N-alpha- | 1458 |
| | acetyltransferase 15, NatA | |
| | auxiliary subun | |
| P62330_C155 | ARF6 ADP-ribosylation factor | 1459 |
| | 6 | |
| Q13501_C27 | SQSTM1 Sequestosome-1 | 1460 |
| Q5VWZ2_C12 | LYPLAL1 | 1461 |
| | Lysophospholipase-like | |
| | protein 1 | |
| Q04206_C105 | RELA Transcription factor p65 | 1462 |
| P36551_C198 | CPOX Coproporphyrinogen- | 1463 |
| | III oxidase, mitochondrial | |
| P25398_C69 | RPS12 40S ribosomal protein | 1464 |
| | S12 | |
| Q00839_C648 | HNRNPU Heterogeneous | 1465 |
| | nuclear ribonucleoprotein U | |
| P24752_C142 | ACAT1 Acetyl-CoA | 1466 |
| | acetyltransferase, | |
| | mitochondrial | |
| P02545_C522 | LMNA Prelamin-A/C | 1467 |
| O60763_C678 | USO1 General vesicular | 1468 |
| | transport factor p115 | |
| P04637_C182 | TP53 Cellular tumor antigen | 1469 |
| | p53 | |
| Q9UHX1_C129 | PUF60 Poly(U)-binding- | 1470 |
| | splicing factor PUF60 | |
| Q9Y383_C348 | LUC7L2 Putative RNA- | 1471 |
| | binding protein Luc7-like 2 | |
| P78371_C289 | CCT2 T-complex protein 1 | 1472 |
| | subunit beta | |
| Q15149_C4494 | PLEC Plectin | 1473 |
| P13639_C369 | EEF2 Elongation factor 2 | 1474 |
| P61163_C34 | ACTR1A Alpha-centractin | 1475 |
| Q7Z2W4_C645 | ZC3HAV1 Zinc finger CCCH- | 1476 |
| | type antiviral protein 1 | |
| P23526_C421 | AHCY | 1477 |
| | Adenosylhomocysteinase | |
| Q9P2T1_C348 | GMPR2 GMP reductase 2 | 1478 |
| Q12849_C476 | GRSF1 G-rich sequence factor | 1479 |
| | 1 | |
| P07814_C744 | EPRS Bifunctional | 1480 |
| | glutamate/proline--tRNA | |
| | ligase | |
| Q9ULA0_C327 | DNPEP Aspartyl | 1481 |
| | aminopeptidase | |
| Q01081_C67 | U2AF1 Splicing factor U2AF | 1482 |
| | 35 kDa subunit | |
| P53041_C11 | PPP5C Serine/threonine- | 1483 |
| | protein phosphatase 5 | |
| P63244_C138 | GNB2L1 Guanine nucleotide- | 1484 |
| | binding protein subunit beta-2- | |
| Q06323_C106 | PSME1 Proteasome activator | 1485 |
| | complex subunit 1 | |
| O95071_C730 | UBR5 E3 ubiquitin-protein | 1486 |
| | ligase UBR5 | |
| Q9H0D6_C736 | XRN2 5-3 exoribonuclease 2 | 1487 |
| P00558_C50 | PGK1 Phosphoglycerate | 1488 |
| | kinase 1 | |
| P52272_C114 | HNRNPM Heterogeneous | 1489 |
| | nuclear ribonucleoprotein M | |
| Q92598_C34 | HSPH1 Heat shock protein 105 | 1490 |
| | kDa | |
| P51858_C12 | HDGF Hepatoma-derived | 1491 |
| | growth factor | |
| Q9UBN7_C426 | HDAC6 Histone deacetylase 6 | 1492 |
| P09234_C25 | SNRPC U1 small nuclear | 1493 |
| | ribonucleoprotein C | |
| Q9P1F3_C39 | ABRACL Costars family | 1494 |
| | protein ABRACL | |
| P61158_C34 | ACTR3 Actin-related protein 3 | 1495 |
| P13639_C812 | EEF2 Elongation factor 2 | 1496 |
| B0V043_C41 | VARS Valyl-tRNA synthetase | 1497 |
| Q9NQT5_C215 | EXOSC3 Exosome complex | 1498 |
| | component RRP40 | |
| Q6PJG6_C539 | BRAT1 BRCA1-associated | 1499 |
| | ATM activator 1 | |
| H3BN98_C196 | Uncharacterized protein | 1500 |
| P35998_C389 | PSMC2 26S protease | 1501 |
| | regulatory subunit 7 | |
| P41250_C616 | GARS Glycine--tRNA ligase | 1502 |
| Q9Y5Y2_C269 | NUBP2 Cytosolic Fe—S cluster | 1503 |
| | assembly factor NUBP2 | |
| P31948_C26 | STIP1 Stress-induced- | 1504 |
| | phosphoprotein 1 | |
| Q99575_C705 | POP1 Ribonucleases P/MRP | 1505 |
| | protein subunit POP1 | |
| O00429_C470 | DNM1L Dynamin-1-like | 1506 |
| | protein | |
| O43264_C568 | ZW10 Centromere/kinetochore | 1507 |
| | protein zw10 homolog | |
| Q99961_C277 | SH3GL1 Endophilin-A2 | 1508 |
| Q99460_C898 | PSMD1 26S proteasome non- | 1509 |
| | ATPase regulatory subunit 1 | |
| P49411_C290 | TUFM Elongation factor Tu, | 1510 |
| | mitochondrial | |
| P21980_C370 | TGM2 Protein-glutamine | 1511 |
| | gamma-glutamyltransferase 2 | |
| P21980_C545 | TGM2 Protein-glutamine | 1512 |
| | gamma-glutamyltransferase 2 | |
| O00273_C154 | DFFA DNA fragmentation | 1513 |
| | factor subunit alpha | |
| P09936_C152 | UCHL1 Ubiquitin carboxyl- | 1514 |
| | terminal hydrolase isozyme L1 | |
| Q66K74_C440 | MAP1S Microtubule- | 1515 |
| | associated protein 1S | |
| Q9Y305_C155 | ACOT9 Acyl-coenzyme A | 1516 |
| | thioesterase 9, mitochondrial | |
| P17655_C105 | CAPN2 Calpain-2 catalytic | 1517 |
| | subunit | |
| P17655_C301 | CAPN2 Calpain-2 catalytic | 1518 |
| | subunit | |
| P35611_C525 | ADD1 Alpha-adducin | 1519 |
| P14625_C645 | HSP90B1 Endoplasmin | 1520 |
| O95571_C34 | ETHE1 Protein ETHE1, | 1521 |
| | mitochondrial | |
| P62241_C100 | RPS8 40S ribosomal protein | 1522 |
| | S8 | |
| P22061_C95 | PCMT1 Protein-L- | 1523 |
| | isoaspartate(D-aspartate) O- | |
| | methyltransferase | |
| P19367_C813 | HK1 Hexokinase-1 | 1524 |
| P20073_C363 | ANXA7 Annexin A7 | 1525 |
| O95486_C704 | SEC24A Protein transport | 1526 |
| | protein Sec24A | |
| P27816_C535 | MAP4 Microtubule-associated | 1527 |
| | protein 4 | |
| Q08J23_C93 | NSUN2 tRNA (cytosine(34)- | 1528 |
| | C(5))-methyltransferase | |
| Q9NSD9_C195 | FARSB Phenylalanine--tRNA | 1529 |
| | ligase beta subunit | |
| Q9GZZ9_C250 | UBA5 Ubiquitin-like modifier- | 1530 |
| | activating enzyme 5 | |
| P41240_C290 | CSK Tyrosine-protein kinase | 1531 |
| | CSK | |
| P00492_C23 | HPRT1 Hypoxanthine-guanine | 1532 |
| | phosphoribosyltransferase | |
| Q99714_C58 | HSD17B10 3-hydroxyacyl- | 1533 |
| | CoA dehydrogenase type-2 | |
| P27707_C59 | DCK Deoxycytidine kinase | 1534 |
| P19784_C336 | CSNK2A2 Casein kinase II | 1535 |
| | subunit alpha | |
| P50914_C42 | RPL14 60S ribosomal protein | 1536 |
| | L14 | |
| Q8WVJ2_C14 | NUDCD2 NudC domain- | 1537 |
| | containing protein 2 | |
| Q02543_C22 | RPL18A 60S ribosomal | 1538 |
| | protein L18a | |
| Q6YN16_C218 | HSDL2 Hydroxysteroid | 1539 |
| | dehydrogenase-like protein 2 | |
| Q9UHD8_C248 | SEPT9 Septin-9 | 1540 |
| P22314_C1040 | UBA1 Ubiquitin-like modifier- | 1541 |
| | activating enzyme 1 | |
| Q15020_C670 | SART3 Squamous cell | 1542 |
| | carcinoma antigen recognized | |
| | by T-cells 3 | |
| O15144_C120 | ARPC2 Actin-related protein | 1543 |
| | 2/3 complex subunit 2 | |
| Q9UNM6_C357 | PSMD13 26S proteasome non- | 1544 |
| | ATPase regulatory subunit 13 | |
| P30040_C157 | ERP29 Endoplasmic reticulum | 1545 |
| | resident protein 29 | |
| Q96Q11_C373 | TRNT1 CCA tRNA | 1546 |
| | nucleotidyltransferase 1, | |
| | mitochondrial | |
| Q02750_C277 | MAP2K1 Dual specificity | 1547 |
| | mitogen-activated protein | |
| | kinase | |
| P27816_C635 | MAP4 Microtubule-associated | 1548 |
| | protein 4 | |
| Q13185_C69 | CBX3 Chromobox protein | 1549 |
| | homolog 3 | |
| P21291_C40 | CSRP1 Cysteine and glycine- | 1550 |
| | rich protein 1 | |
| P55072_C535 | VCP Transitional endoplasmic | 1551 |
| | reticulum ATPase | |
| P55072_C572 | VCP Transitional endoplasmic | 1552 |
| | reticulum ATPase | |
| P39687_C87 | ANP32A Acidic leucine-rich | 1553 |
| | nuclear phosphoprotein 32 | |
| | family | |
| Q9Y314_C8 | NOSIP Nitric oxide synthase- | 1554 |
| | interacting protein | |
| Q9UBT2_C173 | UBA2 SUMO-activating | 1555 |
| | enzyme subunit 2 | |
| P34897_C91 | SHMT2 Serine | 1556 |
| | hydroxymethyltransferase, | |
| | mitochondrial | |
| O00299_C178 | CLIC1 Chloride intracellular | 1557 |
| | channel protein 1 | |
| Q9Y570_C238 | PPME1 Protein phosphatase | 1558 |
| | methylesterase 1 | |
| P61927_C19 | RPL37 60S ribosomal protein | 1559 |
| | L37 | |
| O75153_C1196 | KIAA0664 Clustered | 1560 |
| | mitochondria protein homolog | |
| P23919_C31 | DTYMK Thymidylate kinase | 1561 |
| P40121_C290 | CAPG Macrophage-capping | 1562 |
| | protein | |
| A0AVT1_C433 | UBA6 Ubiquitin-like modifier- | 1563 |
| | activating enzyme 6 | |
| Q15365_C201 | PCBP1 Poly(rC)-binding | 1564 |
| | protein 1 | |
| P29401_C386 | TKT Transketolase | 1565 |
| P48147_C25 | PREP Prolyl endopeptidase | 1566 |
| Q01813_C411 | PFKP 6-phosphofructokinase | 1567 |
| | type C | |
| P21333_C2543 | FLNA Filamin-A | 1568 |
| P21333_C2601 | FLNA Filamin-A | 1569 |
| O00154_C288 | ACOT7 Cytosolic acyl | 1570 |
| | coenzyme A thioester | |
| | hydrolase | |
| P27635_C195 | RPL10 60S ribosomal protein | 1571 |
| | L10 | |
| P26038_C117 | MSN Moesin | 1572 |
| Q96HE7_C208 | ERO1L ERO1-like protein | 1573 |
| | alpha | |
| P12814_C480 | ACTN1 Alpha-actinin-1 | 1574 |
| P07203_C202 | GPX1 Glutathione peroxidase | 1575 |
| | 1 | |
| Q99497_C46 | PARK7 Protein DJ-1 | 1576 |
| Q9HAV4_C1131 | XPO5 Exportin-5 | 1577 |
| Q9UJX3_C131 | ANAPC7 Anaphase-promoting | 1578 |
| | complex subunit 7 | |
| O75821_C139 | EIF3G Eukaryotic translation | 1579 |
| | initiation factor 3 subunit | |
| P00338_C131 | LDHA L-lactate | 1580 |
| | dehydrogenase A chain | |
| Q15102_C55 | PAFAH1B3 Platelet-activating | 1581 |
| | factor acetylhydrolase IB subu | |
| Q1KMD3_C308 | HNRNPUL2 Heterogeneous | 1582 |
| | nuclear ribonucleoprotein U- | |
| | like protein 2 | |
| P54886_C88 | ALDH18A1 Delta-1-pyrroline- | 1583 |
| | 5-carboxylate synthase | |
| Q5VSL9_C798 | FAM40A Protein FAM40A | 1584 |
| P30084_C213 | ECHS1 Enoyl-CoA hydratase, | 1585 |
| | mitochondrial | |
| P30086_C168 | PEBP1 | 1586 |
| | Phosphatidylethanolamine- | |
| | binding protein 1 | |
| Q9Y3D0_C158 | FAM96B Mitotic spindle- | 1587 |
| | associated MMXD complex | |
| | subunit MI | |
| P49589_C204 | CARS Cysteine--tRNA ligase, | 1588 |
| | cytoplasmic | |
| Q15366_C217 | PCBP2 Poly(rC)-binding | 1589 |
| | protein 2 | |
| O75369_C2556 | FLNB Filamin-B | 1590 |
| Q16181_C126 | SEPT7 Septin-7 | 1591 |
| Q99661_C245 | KIF2C Kinesin-like protein | 1592 |
| | KIF2C | |
| Q9UHD1_C86 | CHORDC1 Cysteine and | 1593 |
| | histidine-rich domain- | |
| | containing protein | |
| P0CB43_C368 | FAM203B Protein FAM203B | 1594 |
| P40925_C137 | MDH1 Malate dehydrogenase, | 1595 |
| | cytoplasmic | |
| Q12888_C1933 | TP53BP1 Tumor suppressor | 1596 |
| | p53-binding protein 1 | |
| Q14247_C112 | CTTN Src substrate cortactin | 1597 |
| Q13200_C251 | PSMD2 26S proteasome non- | 1598 |
| | ATPase regulatory subunit 2 | |
| P13639_C591 | EEF2 Elongation factor 2 | 1599 |
| P27816_C126 | MAP4 Microtubule-associated | 1600 |
| | protein 4 | |
| P48739_C187 | PITPNB Phosphatidylinositol | 1601 |
| | transfer protein beta isoform | |
| P14866_C472 | HNRNPL Heterogeneous | 1602 |
| | nuclear ribonucleoprotein L | |
| P07237_C343 | P4HB Protein disulfide- | 1603 |
| | isomerase | |
| O43708_C205 | GSTZ1 Maleylacetoacetate | 1604 |
| | isomerase | |
| P19623_C25 | SRM Spermidine synthase | 1605 |
| P56537_C56 | EIF6 Eukaryotic translation | 1606 |
| | initiation factor 6 | |
| Q96SW2_C188 | CRBN Protein cereblon | 1607 |
| P41240_C31 | CSK Tyrosine-protein kinase | 1608 |
| | CSK | |
| P36551_C127 | CPOX Coproporphyrinogen- | 1609 |
| | III oxidase, mitochondrial | |
| P52292_C133 | KPNA2 Importin subunit | 1610 |
| | alpha-2 | |
| Q04637_C1516 | EIF4G1 Eukaryotic translation | 1611 |
| | initiation factor 4 gamma 1 | |
| P60981_C80 | DSTN Destrin | 1612 |
| Q7RTV0_C40 | PHF5A PHD finger-like | 1613 |
| | domain-containing protein 5A | |
| P19838_C61 | NFKB1 Nuclear factor NF- | 1614 |
| | kappa-B p105 subunit | |
| P53396_C633 | ACLY ATP-citrate synthase | 1615 |
| P49207_C83 | RPL34 60S ribosomal protein | 1616 |
| | L34 | |
| Q14974_C689 | KPNB1 Importin subunit beta- | 1617 |
| | 1 | |
| Q96I99_C162 | SUCLG2 Succinyl-CoA ligase | 1618 |
| P51610_C1139 | HCFC1 Host cell factor 1 | 1619 |
| Q14657_C23 | LAGE3 L antigen family | 1620 |
| | member 3 | |
| P13639_C290 | EEF2 Elongation factor 2 | 1621 |
| P22314_C481 | UBA1 Ubiquitin-like modifier- | 1622 |
| | activating enzyme 1 | |
| Q52LJ0_C295 | FAM98B Protein FAM98B | 1623 |
| Q7Z2W4_C38 | ZC3HAV1 Zinc finger CCCH- | 1624 |
| | type antiviral protein 1 | |
| P26447_C81 | S100A4 Protein S100-A4 | 1625 |
| P30041_C47 | PRDX6 Peroxiredoxin-6 | 1626 |
| P04040_C377 | CAT Catalase | 1627 |
| Q14847_C20 | LASP1 LIM and SH3 domain | 1628 |
| | protein 1 | |
| P55209_C88 | NAP1L1 Nucleosome | 1629 |
| | assembly protein 1-like 1 | |
| Q9NRP4_C80 | ACN9 Protein ACN9 | 1630 |
| | homolog, mitochondrial | |
| P17987_C385 | TCP1 T-complex protein 1 | 1631 |
| | subunit alpha | |
| P13797_C104 | PLS3 Plastin-3 | 1632 |
| Q7Z434_C33 | MAVS Mitochondrial | 1633 |
| | antiviral-signaling protein | |
| P49915_C489 | GMPS GMP synthase K | 1634 |
| Q9H814_C51 | PHAX Phosphorylated adapter | 1635 |
| | RNA export protein | |
| Q15181_C274 | PPA1 Inorganic | 1636 |
| | pyrophosphatase | |
| Q9UPN7_C795 | PPP6R1 Serine/threonine- | 1637 |
| | protein phosphatase 6 | |
| | regulatory | |
| P50395_C202 | GDI2 Rab GDP dissociation | 1638 |
| | inhibitor beta | |
| P27695_C296 | APEX1 DNA-(apurinic or | 1639 |
| | apyrimidinic site) lyase | |
| P13010_C493 | XRCC5 X-ray repair cross- | 1640 |
| | complementing protein 5 | |
| P27635_C49 | RPL10 60S ribosomal protein | 1641 |
| | L10 | |
| Q9BZE9_C109 | ASPSCR1 Tether containing | 1642 |
| | UBX domain for GLUT4 | |
| P43487_C99 | RANBP1 Ran-specific | 1643 |
| | GTPase-activating protein | |
| P78527_C1904 | PRKDC DNA-dependent | 1644 |
| | protein kinase catalytic subunit | |
| Q6JBY9_C155 | RCSD1 CapZ-interacting | 1645 |
| | protein | |
| P14625_C576 | HSP90B1 Endoplasmin | 1646 |
| Q8N806_C260 | UBR7 Putative E3 ubiquitin- | 1647 |
| | protein ligase UBR7 | |
| Q9GZT9_C127 | EGLN1 Egl nine homolog 1 | 1648 |
| P06744_C404 | GPI Glucose-6-phosphate | 1649 |
| | isomerase | |
| P00390_C102 | GSR Glutathione reductase, | 1650 |
| | mitochondrial | |
| P25398_C106 | RPS12 40S ribosomal protein | 1651 |
| | S12 | |
| Q00839_C497 | HNRNPU Heterogeneous | 1652 |
| | nuclear ribonucleoprotein U | |
| P49368_C455 | CCT3 T-complex protein 1 | 1653 |
| | subunit gamma | |
| P33993_C482 | MCM7 DNA replication | 1654 |
| | licensing factor MCM7 | |
| P57764_C191 | GSDMD Gasdermin-D | 1655 |
| P40222_C523 | TXLNA Alpha-taxilin | 1656 |
| Q9NTZ6_C887 | RBM12 RNA-binding protein | 1657 |
| | 12 | |
| Q9Y617_C80 | PSAT1 Phosphoserine | 1658 |
| | aminotransferase | |
| P27695_C99 | APEX1 DNA-(apurinic or | 1659 |
| | apyrimidinic site) lyase | |
| Q7Z6Z7_C3239 | HUWE1 E3 ubiquitin-protein | 1660 |
| | ligase HUWE1 | |
| Q16644_C203 | MAPKAPK3 MAP kinase- | 1661 |
| | activated protein kinase 3 | |
| O15355_C13 | PPM1G Protein phosphatase | 1662 |
| | 1G | |
| Q15149_C3821 | PLEC Plectin | 1663 |
| O94776_C209 | MTA2 Metastasis-associated | 1664 |
| | protein MTA2 | |
| A6NHG4_C24 | DDTL D-dopachrome | 1665 |
| | decarboxylase-like protein | |
| P356I1_C430 | ADD1 Alpha-adducin | 1666 |
| Q9NVA2_C41 | SEPT11 Septin-11 | 1667 |
| P19404_C225 | NDUFV2 NADH | 1668 |
| | dehydrogenase | |
| P35579_C569 | MYH9 Myosin-9 | 1669 |
| P19367_C834 | HK1 Hexokinase-1 | 1670 |
| P30041_C91 | PRDX6 Peroxiredoxin-6 | 1671 |
| P21281_C162 | ATP6V1B2 V-type proton | 1672 |
| | ATPase subunit B, brain | |
| | isoform | |
| P13796_C42 | LCP1 Plastin-2 | 1673 |
| O00410_C687 | IPO5 Importin-5 | 1674 |
| P49207_C49 | RPL34 60S ribosomal protein | 1675 |
| | L34 | |
| P35658_C186 | NUP214 Nuclear pore | 1676 |
| | complex protein Nup214 | |
| P17858_C170 | PFKL 6-phosphofructokinase, | 1677 |
| | liver type | |
| P21333_C574 | FLNA Filamin-A | 1678 |
| O60701_C241 | UGDH UDP-glucose 6- | 1679 |
| | dehydrogenase | |
| Q15691_C228 | MAPRE1 Microtubule- | 1680 |
| | associated protein RP/EB | |
| | family member | |
| P31153_C104 | MAT2A S- | 1681 |
| | adenosylmethionine synthase | |
| | isoform type-2 | |
| Q14247_C246 | CTTN Src substrate cortactin | 1682 |
| Q8IWX8_C69 | CHERP Calcium homeostasis | 1683 |
| | endoplasmic reticulum protein | |
| Q99543_C394 | DNAJC2 DnaJ homolog | 1684 |
| | subfamily C member 2 | |
| Q6JBY9_C181 | RCSD1 CapZ-interacting | 1685 |
| | protein | |
| P12814_C41 | ACTN1 Alpha-actinin-1 | 1686 |
| P17812_C491 | CTPS1 CTP synthase 1 | 1687 |
| E7ETI0_C21 | ARPC4-TTLL3 Protein | 1688 |
| | ARPC4-TTLL3 | |
| Q9ULC4_C14 | MCTS1 Malignant T-cell- | 1689 |
| | amplified sequence 1 | |
| P08134_C16 | RHOC Rho-related GTP- | 1690 |
| | binding protein RhoC | |
| Q14980_C80 | NUMA1 Nuclear mitotic | 1691 |
| | apparatus protein 1 | |
| Q9NYL9_C231 | TMOD3 Tropomodulin-3 | 1692 |
| Q99439_C61 | CNN2 Calponin-2 | 1693 |
| P46776_C144 | RPL27A 60S ribosomal | 1694 |
| | protein L27a | |
| O14980_C164 | XPO1 Exportin-1 | 1695 |
| Q9Y2L1_C213 | DIS3 Exosome complex | 1696 |
| | exonuclease RRP44 | |
| O60502_C596 | MGEA5 Bifunctional protein | 1697 |
| | NCOAT | |
| P49748_C237 | ACADVL Very long-chain | 1698 |
| | specific acyl-CoA | |
| | dehydrogenase, mitochondrial | |
| O76003_C146 | GLRX3 Glutaredoxin-3 | 1699 |
| P27797_C163 | CALR Calreticulin | 1700 |
| P49368_C279 | CCT3 T-complex protein 1 | 1701 |
| | subunit gamma | |
| P32119_C172 | PRDX2 Peroxiredoxin-2 | 1702 |
| O15541_C15 | RNF113A RING finger protein | 1703 |
| | 113A | |
| Q15417_C59 | CNN3 Calponin-3 | 1704 |
| P60981_C135 | DSTN Destrin | 1705 |
| P36776_C682 | LONP1 Lon protease homolog, | 1706 |
| | mitochondrial | |
| P13693_C28 | TPT1 Translationally- | 1707 |
| | controlled tumor protein | |
| B5ME19_C444 | EIF3CL Eukaryotic translation | 1708 |
| | initiation factor 3 subunit | |
| Q93009_C223 | USP7 Ubiquitin carboxyl- | 1709 |
| | terminal hydrolase 7 | |
| Q9UPQ0_C669 | LIMCH1 LIM and calponin | 1710 |
| | homology domains-containing | |
| | prote | |
| Q96EY4_C162 | TMA16 Translation | 1711 |
| | machinery-associated protein | |
| | 16 | |
| Q9BSH5_C243 | HDHD3 Haloacid | 1712 |
| | dehalogenase-like hydrolase | |
| | domain-contai | |
| Q99832_C450 | CCT7 T-complex protein I | 1713 |
| | subunit eta | |
| P68366_C129 | TUBA4A Tubulin alpha-4A | 1714 |
| | chain | |
| Q6IS14_C73 | EIF5AL1 Eukaryotic | 1715 |
| | translation initiation factor 5A- | |
| | 1-like | |
| P52907_C157 | CAPZA1 F-actin-capping | 1716 |
| | protein subunit alpha-1 | |
| P78527_C25 | PRKDC DNA-dependent | 1717 |
| | protein kinase catalytic subunit | |
| O43592_C650 | XPOT Exportin-T | 1718 |
| P49459_C88 | UBE2A Ubiquitin-conjugating | 1719 |
| | enzyme E2 A | |
| Q9UEW8_C237 | STK39 STE20/SPS1-related | 1720 |
| | proline-alanine-rich protein ki | |
| Q92888_C537 | ARHGEF1 Rho guanine | 1721 |
| | nucleotide exchange factor 1 | |
| P17812_C362 | CTPS1 CTP synthase 1 | 1722 |
| Q8IY67_C255 | RAVER1 Ribonucleoprotein | 1723 |
| | PTB-binding 1 | |
| Q13547_C408 | HDAC1 Histone deacetylase 1 | 1724 |
| P00505_C106 | GOT2 Aspartate | 1725 |
| | aminotransferase, | |
| | mitochondrial | |
| P35579_C816 | MYH9 Myosin-9 | 1726 |
| O00148_C197 | DDX39A ATP-dependent | 1727 |
| | RNA helicase DDX39A | |
| Q14192_C150 | FHL2 Four and a half LIM | 1728 |
| | domains protein 2 | |
| Q14204_C633 | DYNC1H1 Cytoplasmic | 1729 |
| | dynein 1 heavy chain 1 | |
| P07814_C1487 | EPRS Bifunctional | 1730 |
| | glutamate/proline--tRNA | |
| | ligase | |
| P27816_C1098 | MAP4 Microtubule-associated | 1731 |
| | protein 4 | |
| P61586_C107 | RHOA Transforming protein | 1732 |
| | RhoA | |
| Q08J23_C673 | NSUN2 tRNA (cytosine(34)- | 1733 |
| | C(5))-methyltransferase | |
| P53041_C77 | PPP5C Serine/threonine- | 1734 |
| | protein phosphatase 5 | |
| O43707_C173 | ACTN4 Alpha-actinin-4 | 1735 |
| Q06210_C55 | GFPT1 Glucosamine-- | 1736 |
| | fructose-6-phosphate | |
| | aminotransferase | |
| P06400_C853 | RB1 Retinoblastoma- | 1737 |
| | associated protein | |
| Q9BUJ2_C391 | HNRNPUL1 Heterogeneous | 1738 |
| | nuclear ribonucleoprotein U- | |
| | like protein | |
| E7EVH7_C286 | KLC1 Kinesin light chain 1 | 1739 |
| Q68CZ2_C1241 | TNS3 Tensin-3 | 1740 |
| Q9Y5Y2_C72 | NUBP2 Cytosolic Fe—S cluster | 1741 |
| | assembly factor NUBP2 | |
| P11498_C850 | PC Pyruvate carboxylase, | 1742 |
| | mitochondrial | |
| Q9BXS6_C256 | NUSAP1 Nucleolar and | 1743 |
| | spindle-associated protein 1 | |
| P56192_C38 | MARS Methionine--tRNA | 1744 |
| | ligase, cytoplasmic | |
| P35914_C323 | HMGCL | 1745 |
| | Hydroxymethylglutaryl-CoA | |
| | lyase, mitochondrial | |
| P00367_C172 | GLUD1 Glutamate | 1746 |
| | dehydrogenase 1, | |
| | mitochondrial | |
| Q99873_C350 | PRMT1 Protein arginine N- | 1747 |
| | methyltransferase 1 | |
| P15170_C276 | GSPT1 Eukaryotic peptide | 1748 |
| | chain release factor GTP- | |
| | bindin | |
| Q13838_C165 | DDX39B Spliceosome RNA | 1749 |
| | helicase DDX39B | |
| O75369_C1617 | FLNB Filamin-B | 1750 |
| Q96RU3_C609 | FNBP1 Formin-binding | 1751 |
| | protein 1 | |
| P16333_C266 | NCK1 Cytoplasmic protein | 1752 |
| | NCK1 | |
| P35250_C88 | RFC2 Replication factor C | 1753 |
| | subunit 2 | |
| Q9BSD7_C184 | NTPCR Cancer-related | 1754 |
| | nucleoside-triphosphatase | |
| P13489_C142 | RNH1 Ribonuclease inhibitor | 1755 |
| P26639_C343 | TARS Threonine--tRNA | 1756 |
| | ligase, cytoplasmic | |
| P31153_C56 | MAT2A S- | 1757 |
| | adenosylmethionine synthase | |
| | isoform type-2 | |
| P01591_C131 | IGJ Immunoglobulin J chain | 1758 |
| P07858_C93 | CTSB Cathepsin B | 1759 |
| P62191_C399 | PSMC1 26S protease | 1760 |
| | regulatory subunit 4 | |
| P85037_C439 | FOXK1 Forkhead box protein | 1761 |
| | K1 | |
| Q96EY7_C642 | PTCD3 Pentatricopeptide | 1762 |
| | repeat-containing protein 3, | |
| | mitochondrial | |
| P78417_C192 | GSTO1 Glutathione S- | 1763 |
| | transferase omega-1 | |
| O75694_C704 | NUP155 Nuclear pore | 1764 |
| | complex protein Nup155 | |
| Q9NZD2_C36 | GLTP Glycolipid transfer | 1765 |
| | protein | |
| Q9NR45_C287 | NANS Sialic acid synthase | 1766 |
| P52788_C318 | SMS Spermine synthase | 1767 |
| P52788_C337 | SMS Spermine synthase | 1768 |
| P37837_C250 | TALDO1 Transaldolase | 1769 |
| Q9UBB4_C283 | ATXN10 Ataxin-10 | 1770 |
| Q14204_C1888 | DYNC1H1 Cytoplasmic | 1771 |
| | dynein 1 heavy chain 1 | |
| P14866_C452 | HNRNPL Heterogeneous | 1772 |
| | nuclear ribonucleoprotein L | |
| P56537_C15 | EIF6 Eukaryotic translation | 1773 |
| | initiation factor 6 | |
| Q9Y5Y2_C54 | NUBP2 Cytosolic Fe—S cluster | 1774 |
| | assembly factor NUBP2 | |
| P28838_C445 | LAP3 Cytosol aminopeptidase | 1775 |
| P31350_C317 | RRM2 Ribonucleoside- | 1776 |
| | diphosphate reductase subunit | |
| | M2 | |
| Q04637_C1384 | EIF4G1 Eukaryotic translation | 1777 |
| | initiation factor 4 gamma 1 | |
| P63167_C56 | DYNLL1 Dynein light chain 1, | 1778 |
| | cytoplasmic | |
| Q86WR0_C83 | CCDC25 Coiled-coil domain- | 1779 |
| | containing protein 25 | |
| P41227_C194 | NAA10 N-alpha- | 1780 |
| | acetyltransferase 10 | |
| Q9BY42_C262 | C20orf43 UPF0549 protein | 1781 |
| | C20orf43 | |
| Q9NVP2_C172 | ASF1B Histone chaperone | 1782 |
| | ASF1B | |
| Q8IZ07_C540 | ANKRD13A Ankyrin repeat | 1783 |
| | domain-containing protein 13A | |
| Q8IUI8_C154 | CRLF3 Cytokine receptor-like | 1784 |
| | factor 3 | |
| Q9H6S3_C543 | EPS8L2 Epidermal growth | 1785 |
| | factor receptor kinase substrate | |
| Q06124_C573 | PTPN11 Tyrosine-protein | 1786 |
| | phosphatase non-receptor type | |
| | 11 | |
| P62829_C125 | RPL23 60S ribosomal protein | 1787 |
| | L23 | |
| P55769_C93 | NHP2L1 NHP2-like protein 1 | 1788 |
| Q15233_C208 | NONO Non-POU domain- | 1789 |
| | containing octamer-binding | |
| | protein | |
| Q13200_C459 | PSMD2 26S proteasome non- | 1790 |
| | ATPase regulatory subunit 2 | |
| Q9ULZ2_C269 | STAP1 Signal-transducing | 1791 |
| | adaptor protein 1 | |
| P78527_C3403 | PRKDC DNA-dependent | 1792 |
| | protein kinase catalytic subunit | |
| Q9NZ63_C145 | C9orf78 Uncharacterized | 1793 |
| | protein C9orf78 | |
| P22234_C350 | PAICS Multifunctional protein | 1794 |
| | ADE2 | |
| Q9NX46_C132 | ADPRHL2 Poly(ADP-ribose) | 1795 |
| | glycohydrolase ARH3 | |
| Q6PJT7_C261 | ZC3H14 Zinc finger CCCH | 1796 |
| | domain-containing protein 14 | |
| P52789_C909 | HK2 Hexokinase-2 | 1797 |
| O00148_C86 | DDX39A ATP-dependent | 1798 |
| | RNA helicase DDX39A | |
| P48643_C302 | CCT5 T-complex protein 1 | 1799 |
| | subunit epsilon | |
| Q9H6Z4_C203 | RANBP3 Ran-binding protein | 1800 |
| | 3 | |
| P33991_C605 | MCM4 DNA replication | 1801 |
| | licensing factor MCM4 | |
| Q13330_C229 | MTA1 Metastasis-associated | 1802 |
| | protein MTA1 | |
| O95630_C264 | STAMBP STAM-binding | 1803 |
| | protein | |
| P25786_C85 | PSMA1 Proteasome subunit | 1804 |
| | alpha type-1 | |
| P42704_C1277 | LRPPRC Leucine-rich PPR | 1805 |
| | motif-containing protein, | |
| | mitochondrial | |
| Q9NR50_C106 | EIF2B3 Translation initiation | 1806 |
| | factor eIF-2B subunit gamma | |
| Q9H299_C71 | SH3BGRL3 SH3 domain- | 1807 |
| | binding glutamic acid-rich-like | |
| | protein | |
| Q32MZ4_C14 | LRRFIP1 Leucine-rich repeat | 1808 |
| | flightless-interacting protein | |
| Q13619_C633 | CUL4A Cullin-4A | 1809 |
| O75832_C107 | PSMD10 26S proteasome non- | 1810 |
| | ATPase regulatory subunit 10 | |
| O75874_C379 | IDH1 Isocitrate dehydrogenase | 1811 |
| P04075_C178 | ALDOA Fructose- | 1812 |
| | bisphosphate aldolase A | |
| P34897_C412 | SHMT2 Serine | 1813 |
| | hydroxymethyltransferase, | |
| | mitochondrial | |
| Q9Y490_C1939 | TLN1 Talin-1 | 1814 |
| P26358_C896 | DNMT1 DNA (cytosine-5)- | 1815 |
| | methyltransferase 1 | |
| P52701_C88 | MSH6 DNA mismatch repair | 1816 |
| | protein Msh6 | |
| Q96G03_C573 | PGM2 Phosphoglucomutase-2 | 1817 |
| O00410_C1078 | IPO5 Importin-5 | 1818 |
| Q96FW1_C91 | OTUB1 Ubiquitin thioesterase | 1819 |
| | OTUB1 | |
| P02545_C591 | LMNA Prelamin-A/C | 1820 |
| Q7Z5K2_C94 | WAPAL Wings apart-like | 1821 |
| | protein homolog | |
| O75369_C991 | FLNB Filamin-B | 1822 |
| Q969T9_C80 | WBP2 WW domain-binding | 1823 |
| | protein 2 | |
| Q9H773_C162 | DCTPP1 dCTP | 1824 |
| | pyrophosphatase 1 | |
| Q15424_C225 | SAFB Scaffold attachment | 1825 |
| | factor B1 | |
| P07900_C529 | HSP90AA1 Heat shock protein | 1826 |
| | HSP 90-alpha | |
| P23396_C134 | RPS3 40S ribosomal protein | 1827 |
| | S3 | |
| Q8N9N8_C89 | EIF1AD Probable RNA- | 1828 |
| | binding protein EIF1AD | |
| Q12888_C101 | TP53BP1 Tumor suppressor | 1829 |
| | p53-binding protein 1 | |
| P61970_C114 | NUTF2 Nuclear transport | 1830 |
| | factor 2 | |
| P18858_C895 | LIG1 DNA ligase 1 | 1831 |
| P13639_C693 | EEF2 Elongation factor 2 | 1832 |
| Q7L5D6_C160 | GET4 Golgi to ER traffic | 1833 |
| | protein 4 homolog | |
| P63104_C25 | YWHAZ 14-3-3 protein | 1834 |
| | zeta/delta | |
| Q14676_C26 | MDC1 Mediator of DNA | 1835 |
| | damage checkpoint protein 1 | |
| Q15021_C596 | NCAPD2 Condensin complex | 1836 |
| | subunit 1 | |
| O43815_C765 | STRN Striatin | 1837 |
| Q13310_C339 | PABPC4 Polyadenylate- | 1838 |
| | binding protein 4 | |
| P62241_C71 | RPS8 40S ribosomal protein | 1839 |
| | S8 | |
| Q9Y263_C584 | PLAA Phospholipase A-2- | 1840 |
| | activating protein | |
| O95801_C238 | TTC4 Tetratricopeptide repeat | 1841 |
| | protein 4 | |
| Q14192_C254 | FHL2 Four and a half LIM | 1842 |
| | domains protein 2 | |
| Q92841_C584 | DDX17 Probable ATP- | 1843 |
| | dependent RNA helicase | |
| | DDX17 | |
| P07814_C1148 | EPRS Bifunctional | 1844 |
| | glutamate/proline--tRNA | |
| | ligase | |
| P61586_C159 | RHOA Transforming protein | 1845 |
| | RhoA | |
| Q08J23_C221 | NSUN2 tRNA (cytosine(34)- | 1846 |
| | C(5))-methyltransferase | |
| Q9UER7_C699 | DAXX Death domain- | 1847 |
| | associated protein 6 | |
| P05161_C78 | ISG15 Ubiquitin-like protein | 1848 |
| | ISG15 | |
| P07741_C140 | APRT Adenine | 1849 |
| | phosphoribosyltransferase | |
| P08238_C412 | HSP90AB1 Heat shock protein | 1850 |
| | HSP 90-beta | |
| P53597_C60 | SUCLG1 Succinyl-CoA ligase | 1851 |
| Q9UPY8_C182 | MAPRE3 Microtubule- | 1852 |
| | associated protein RP/EB | |
| | family member | |
| P61247_C139 | RPS3A 40S ribosomal protein | 1853 |
| | S3a | |
| Q9Y2L1_C799 | DIS3 Exosome complex | 1854 |
| | exonuclease RRP44 | |
| P23921_C352 | RRM1 Ribonucleoside- | 1855 |
| | diphosphate reductase large | |
| | subunit | |
| P07954_C333 | FH Fumarate hydratase, | 1856 |
| | mitochondrial | |
| O00429_C361 | DNM1L Dynamin-1-like | 1857 |
| | protein | |
| P00491_C31 | PNP Purine nucleoside | 1858 |
| | phosphorylase | |
| P61927_C37 | RPL37 60S ribosomal protein | 1859 |
| | L37 | |
| Q16543_C336 | CDC37 Hsp90 co-chaperone | 1860 |
| | Cdc37 | |
| P49753_C401 | ACOT2 Acyl-coenzyme A | 1861 |
| | thioesterase 2, mitochondrial | |
| Q9BSH4_C256 | TACO1 Translational activator | 1862 |
| | of cytochrome c oxidase 1 | |
| P43686_C379 | PSMC4 26S protease | 1863 |
| | regulatory subunit 6B | |
| O75717_C285 | WDHD1 WD repeat and | 1864 |
| | HMG-box DNA-binding | |
| | protein 1 | |
| P36776_C520 | LONP1 Lon protease homolog, | 1865 |
| | mitochondrial | |
| P36776_C637 | LONP1 Lon protease homolog, | 1866 |
| | mitochondrial | |
| Q16666_C637 | IFI16 Gamma-interferon- | 1867 |
| | inducible protein 16 | |
| Q92619_C1020 | HMHA1 Minor | 1868 |
| | histocompatibility protein HA- | |
| | 1 | |
| Q9P289_C77 | MST4 Serine/threonine-protein | 1869 |
| | kinase MST4 | |
| H3BVE0_C63 | Uncharacterized protein | 1870 |
| P60891_C91 | PRPS1 Ribose-phosphate | 1871 |
| | pyrophosphokinase 1 | |
| Q01813_C343 | PFKP 6-phosphofructokinase | 1872 |
| | type C | |
| P04632_C144 | CAPNS1 Calpain small | 1873 |
| | subunit 1 | |
| Q7Z6Z7_C790 | HUWE1 E3 ubiquitin-protein | 1874 |
| | ligase HUWE1 | |
| O14933_C102 | UBE2L6 Ubiquitin/ISG15- | 1875 |
| | conjugating enzyme E2 L6 | |
| Q8WUA4_C212 | GTF3C2 General transcription | 1876 |
| | factor 3C polypeptide 2 | |
| Q9UJU6_C127 | DBNL Drebrin-like protein | 1877 |
| P21333_C2199 | FLNA Filamin-A | 1878 |
| P45880_C47 | VDAC2 Voltage-dependent | 1879 |
| | anion-selective channel protein | |
| P23396_C119 | RPS3 40S ribosomal protein | 1880 |
| | S3 | |
| P13489_C30 | RNH1 Ribonuclease inhibitor | 1881 |
| P17676_C248 | CEBPB CCAAT/enhancer- | 1882 |
| | binding protein beta | |
| Q92974_C478 | ARHGEF2 Rho guanine | 1883 |
| | nucleotide exchange factor 2 | |
| O75995_C351 | SASH3 SAM and SH3 | 1884 |
| | domain-containing protein 3 | |
| P61978_C184 | HNRNPK Heterogeneous | 1885 |
| | nuclear ribonucleoprotein K | |
| Q15149_C3295 | PLEC Plectin | 1886 |
| P11142_C574 | HSPA8 Heat shock cognate 71 | 1887 |
| | kDa protein | |
| P21964_C223 | COMT Catechol O- | 1888 |
| | methyltransferase | |
| Q96TA1_C466 | FAM129B Niban-like protein | 1889 |
| | 1 | |
| Q9NVG8_C145 | TBC1D13 TBC1 domain | 1890 |
| | family member 13 | |
| Q96EV2_C726 | RBM33 RNA-binding protein | 1891 |
| | 33 | |
| P00505_C295 | GOT2 Aspartate | 1892 |
| | aminotransferase, | |
| | mitochondrial | |
| Q92945_C436 | KHSRP Far upstream element- | 1893 |
| | binding protein 2 | |
| P46734_C227 | MAP2K3 Dual specificity | 1894 |
| | mitogen-activated protein | |
| | kinase | |
| P68104_C370 | EEF1A1 Elongation factor 1- | 1895 |
| | alpha 1 | |
| Q14204_C4216 | DYNC1H1 Cytoplasmic | 1896 |
| | dynein 1 heavy chain 1 | |
| P55060_C939 | CSE1L Exportin-2 | 1897 |
| Q8WYJ6_C293 | SEPT1 Septin-1 | 1898 |
| Q04760_C61 | GLO1 Lactoylglutathione | 1899 |
| | lyase | |
| P54105_C73 | CLNS1A Methylosome | 1900 |
| | subunit pICln | |
| Q99439_C274 | CNN2 Calponin-2 | 1901 |
| Q15054_C137 | POLD3 DNA polymerase delta | 1902 |
| | subunit 3 | |
| Q14232_C169 | EIF2B1 Translation initiation | 1903 |
| | factor eIF-2B subunit alpha | |
| Q8TB45_C284 | DEPTOR DEP domain- | 1904 |
| | containing mTOR-interacting | |
| | protein | |
| P18031_C92 | PTPN1 Tyrosine-protein | 1905 |
| | phosphatase non-receptor type | |
| | 1 | |
| O60343_C74 | TBC1D4 TBC1 domain family | 1906 |
| | member 4 | |
| Q9NQW6_C353 | ANLN Actin-binding protein | 1907 |
| | anillin | |
| Q86U90_C99 | YRDC YrdC domain- | 1908 |
| | containing protein, | |
| | mitochondrial | |
| Q9Y3D2_C45 | MSRB2 Methionine-R- | 1909 |
| | sulfoxide reductase B2, | |
| | mitochondrial | |
| Q9P258_C144 | RCC2 Protein RCC2 | 1910 |
| P56192_C441 | MARS Methionine--tRNA | 1911 |
| | ligase, cytoplasmic | |
| P31751_C311 | AKT2 RAC-beta | 1912 |
| | serine/threonine-protein kinase | |
| O00410_C915 | IPO5 Importin-5 | 1913 |
| Q53ET0_C515 | CRTC2 CREB-regulated | 1914 |
| | transcription coactivator 2 | |
| P29401_C417 | TKT Transketolase | 1915 |
| Q9UPN9_C461 | TRIM33 E3 ubiquitin-protein | 1916 |
| | ligase TRIM33 | |
| P16152_C227 | CBR1 Carbonyl reductase | 1917 |
| P49419_C478 | ALDH7A1 Alpha-aminoadipic | 1918 |
| | semialdehyde dehydrogenase | |
| Q9BX40_C310 | LSM14B Protein LSM14 | 1919 |
| | homolog B | |
| Q93009_C315 | USP7 Ubiquitin carboxyl- | 1920 |
| | terminal hydrolase 7 | |
| Q9P0L0_C60 | VAPA Vesicle-associated | 1921 |
| | membrane protein-associated | |
| | protein A | |
| Q9Y617_C291 | PSAT1 Phosphoserine | 1922 |
| | aminotransferase | |
| Q5U5X0_C97 | LYRM7 LYR motif-containing | 1923 |
| | protein 7 | |
| O75390_C101 | CS Citrate synthase, | 1924 |
| | mitochondrial | |
| P53602_C386 | MVD Diphosphomevalonate | 1925 |
| | decarboxylase | |
| P60953_C157 | CDC42 Cell division control | 1926 |
| | protein 42 homolog | |
| P09936_C47 | UCHL1 Ubiquitin carboxyl- | 1927 |
| | terminal hydrolase isozyme L1 | |
| P21333_C1018 | FLNA Filamin-A | 1928 |
| O00267_C740 | SUPT5H Transcription | 1929 |
| | elongation factor SPT5 | |
| P42166_C518 | TMPO Lamina-associated | 1930 |
| | polypeptide 2, isoform alpha | |
| P41091_C105 | EIF2S3 Eukaryotic translation | 1931 |
| | initiation factor 2 subunit | |
| Q92973_C297 | TNPO1 Transportin-1 | 1932 |
| Q15149_C3336 | PLEC Plectin | 1933 |
| P22314_C278 | UBA1 Ubiquitin-like modifier- | 1934 |
| | activating enzyme 1 | |
| P38117_C42 | ETFB Electron transfer | 1935 |
| | flavoprotein subunit beta | |
| P62316_C46 | SNRPD2 Small nuclear | 1936 |
| | ribonucleoprotein Sm D2 | |
| P48735_C154 | IDH2 Isocitrate dehydrogenase | 1937 |
| O00151_C45 | PDLIM1 PDZ and LIM | 1938 |
| | domain protein 1 | |
| Q8IUD2_C258 | ERC1 ELKS/Rab6- | 1939 |
| | interacting/CAST family | |
| | member 1 | |
| P55210_C186 | CASP7 Caspase-7 | 1940 |
| Q9BY77_C338 | POLDIP3 Polymerase delta- | 1941 |
| | interacting protein 3 | |
| Q13501_C131 | SQSTM1 Sequestosome-1 | 1942 |
| Q96ST2_C749 | IWS1 Protein IWS1 homolog | 1943 |
| P41250_C180 | GARS Glycine--tRNA ligase | 1944 |
| Q96EB6_C67 | SIRT1 NAD-dependent protein | 1945 |
| | deacetylase sirtuin-1 | |
| P23921_C492 | RRM1 Ribonucleoside- | 1946 |
| | diphosphate reductase large | |
| | subunit | |
| Q53H96_C235 | PYCRL Pyrroline-5- | 1947 |
| | carboxylate reductase 3 | |
| B9A064_C213 | IGLL5 Immunoglobulin | 1948 |
| | lambda-like polypeptide 5 | |
| P49588_C947 | AARS Alanine--tRNA ligase, | 1949 |
| | cytoplasmic | |
| P25398_C56 | RPS12 40S ribosomal protein | 1950 |
| | S12 | |
| P28066_C165 | PSMA5 Proteasome subunit | 1951 |
| | alpha type-5 | |
| P46937_C343 | YAP1 Yorkie homolog | 1952 |
| Q16836_C211 | HADH Hydroxyacyl- | 1953 |
| | coenzyme A dehydrogenase, | |
| | mitochondrial | |
| P30626_C57 | SRI Sorcin | 1954 |
| P49023_C108 | PXN Paxillin | 1955 |
| P35270_C159 | SPR Sepiapterin reductase | 1956 |
| P02545_C588 | LMNA Prelamin-A/C | 1957 |
| P08621_C39 | SNRNP70 U1 small nuclear | 1958 |
| | ribonucleoprotein 70 kDa | |
| Q14566_C91 | MCM6 DNA replication | 1959 |
| | licensing factor MCM6 | |
| Q14103_C226 | HNRNPD Heterogeneous | 1960 |
| | nuclear ribonucleoprotein D0 | |
| P26641_C194 | EEF1G Elongation factor 1- | 1961 |
| | gamma | |
| Q9UK41_C128 | VPS28 Vacuolar protein | 1962 |
| | sorting-associated protein 28 | |
| | homolog | |
| Q9BQ52_C441 | ELAC2 Zinc | 1963 |
| | phosphodiesterase ELAC | |
| | protein 2 | |
| P04632_C232 | CAPNS1 Calpain small | 1964 |
| | subunit 1 | |
| P09936_C90 | UCHL1 Ubiquitin carboxyl- | 1965 |
| | terminal hydrolase isozyme L1 | |
| P21333_C1912 | FLNA Filamin-A | 1966 |
| P30566_C340 | ADSL Adenylosuccinate lyase | 1967 |
| O95983_C215 | MBD3 Methyl-CpG-binding | 1968 |
| | domain protein 3 | |
| P40925_C251 | MDH1 Malate dehydrogenase, | 1969 |
| | cytoplasmic | |
| P78417_C90 | GSTO1 Glutathione S- | 1970 |
| | transferase omega-1 | |
| P17655_C82 | CAPN2 Calpain-2 catalytic | 1971 |
| | subunit | |
| O75688_C172 | PPM1B Protein phosphatase | 1972 |
| | 1B | |
| Q09666_C2806 | AHNAK Neuroblast | 1973 |
| | differentiation-associated | |
| | protein AHNA | |
| P62140_C126 | PPP1CB Serine/threonine- | 1974 |
| | protein phosphatase PP1-beta | |
| | cata | |
| P54687_C8 | BCAT1 Branched-chain- | 1975 |
| | amino-acid aminotransferase, | |
| | cytosol | |
| O60934_C487 | NBN Nibrin | 1976 |
| Q9NZT2_C443 | OGFR Opioid growth factor | 1977 |
| | receptor | |
| P52789_C834 | HK2 Hexokinase-2 | 1978 |
| P35579_C671 | MYH9 Myosin-9 | 1979 |
| Q9HAV7_C108 | GRPEL1 GrpE protein | 1980 |
| | homolog 1, mitochondrial | |
| O43175_C18 | PHGDH D-3- | 1981 |
| | phosphoglycerate | |
| | dehydrogenase | |
| O43175_C295 | PHGDH D-3- | 1982 |
| | phosphoglycerate | |
| | dehydrogenase | |
| P12004_C162 | PCNA Proliferating cell | 1983 |
| | nuclear antigen | |
| O75531_C85 | BANF1 Barrier-to- | 1984 |
| | autointegration factor | |
| P46940_C494 | IQGAP1 Ras GTPase- | 1985 |
| | activating-like protein | |
| | IQGAP1 | |
| P11586_C195 | MTHFD1 C-1-tetrahydrofolate | 1986 |
| | synthase, cytoplasmic | |
| Q7Z5L9_C65 | IRF2BP2 Interferon regulatory | 1987 |
| | factor 2-binding protein 2 | |
| Q9Y5K6_C595 | CD2AP CD2-associated | 1988 |
| | protein | |
| Q9Y5L0_C511 | TNPO3 Transportin-3 | 1989 |
| Q9NZN4_C138 | EHD2 EH domain-containing | 1990 |
| | protein 2 | |
| Q13813_C2233 | SPTAN1 Spectrin alpha chain, | 1991 |
| | non-erythrocytic 1 | |
| P62910_C91 | RPL32 60S ribosomal protein | 1992 |
| | L32 | |
| O76003_C229 | GLRX3 Glutaredoxin-3 | 1993 |
| P08559_C181 | PDHA1 Pyruvate | 1994 |
| | dehydrogenase E1 component | |
| | subunit alpha, | |
| P42126_C173 | ECI1 Enoyl-CoA delta | 1995 |
| | isomerase 1, mitochondrial | |
| P84103_C6 | SRSF3 Serine/arginine-rich | 1996 |
| | splicing factor 3 | |
| Q14566_C540 | MCM6 DNA replication | 1997 |
| | licensing factor MCM6 | |
| P21980_C290 | TGM2 Protein-glutamine | 1998 |
| | gamma-glutamyltransferase 2 | |
| O75391_C191 | SPAG7 Sperm-associated | 1999 |
| | antigen 7 | |
| P31943_C22 | HNRNPH1 Heterogeneous | 2000 |
| | nuclear ribonucleoprotein H | |
| P31943_C290 | HNRNPH1 Heterogeneous | 2001 |
| | nuclear ribonucleoprotein H | |
| O95347_C132 | SMC2 Structural maintenance | 2002 |
| | of chromosomes protein 2 | |
| O95983_C172 | MBD3 Methyl-CpG-binding | 2003 |
| | domain protein 3 | |
| P49321_C788 | NASP Nuclear autoantigenic | 2004 |
| | sperm protein | |
| P13489_C96 | RNH1 Ribonuclease inhibitor | 2005 |
| Q86V48_C969 | LUZP1 Leucine zipper protein | 2006 |
| | 1 | |
| Q14240_C135 | EIF4A2 Eukaryotic initiation | 2007 |
| | factor 4A-II | |
| P22307_C71 | SCP2 Non-specific lipid- | 2008 |
| | transfer protein | |
| Q9Y3B4_C83 | SF3B14 Pre-mRNA branch | 2009 |
| | site protein p14 | |
| Q8WUM4_C512 | PDCD6IP Programmed cell | 2010 |
| | death 6-interacting protein | |
| Q8IXH7_C293 | TH1L Negative elongation | 2011 |
| | factor C/D | |
| Q15003_C296 | NCAPH Condensin complex | 2012 |
| | subunit 2 | |
| Q9C0B1_C171 | FTO Alpha-ketoglutarate- | 2013 |
| | dependent dioxygenase FTO | |
| Q8WXI9_C308 | GATAD2B Transcriptional | 2014 |
| | repressor p66-beta | |
| P07814_C856 | EPRS Bifunctional | 2015 |
| | glutamate/proline--tRNA | |
| | ligase | |
| P22102_C134 | GART Trifunctional purine | 2016 |
| | biosynthetic protein adenosin | |
| Q13425_C374 | SNTB2 Beta-2-syntrophin | 2017 |
| Q04760_C139 | GLO1 Lactoylglutathione | 2018 |
| | lyase | |
| P25789_C163 | PSMA4 Proteasome subunit | 2019 |
| | alpha type-4 | |
| P07355_C262 | ANXA2 Annexin A2 | 2020 |
| P53597_C172 | SUCLG1 Succinyl-CoA ligase | 2021 |
| P26368_C429 | U2AF2 Splicing factor U2AF | 2022 |
| | 65 kDa subunit | |
| Q05086_C83 | UBE3A Ubiquitin-protein | 2023 |
| | ligase E3A | |
| O00170_C208 | AIP AH receptor-interacting | 2024 |
| | protein | |
| P31323_C116 | PRKAR2B cAMP-dependent | 2025 |
| | protein kinase type II-beta | |
| | regulat | |
| Q8WW33_C51 | GTSF1 Gametocyte-specific | 2026 |
| | factor 1 | |
| Q9Y5Y2_C196 | NUBP2 Cytosolic Fe—S cluster | 2027 |
| | assembly factor NUBP2 | |
| O95456_C169 | PSMG1 Proteasome assembly | 2028 |
| | chaperone 1 | |
| P13796_C618 | LCP1 Plastin-2 | 2029 |
| P23921_C779 | RRM1 Ribonucleoside- | 2030 |
| | diphosphate reductase large | |
| | subunit | |
| P31689_C149 | DNAJA1 DnaJ homolog | 2031 |
| | subfamily A member 1 | |
| Q9Y490_C1045 | TLN1 Talin-1 | 2032 |
| Q9UGI8_C196 | TES Testin | 2033 |
| P54136_C369 | RARS Arginine--tRNA ligase, | 2034 |
| | cytoplasmic | |
| P47756_C62 | CAPZB F-actin-capping | 2035 |
| | protein subunit beta | |
| P47897_C657 | QARS Glutamine--tRNA | 2036 |
| | ligase | |
| P56192_C12 | MARS Methionine--tRNA | 2037 |
| | ligase, cytoplasmic | |
| Q9Y570_C312 | PPME1 Protein phosphatase | 2038 |
| | methylesterase 1 | |
| Q7L4I2_C382 | RSRC2 Arginine/serine-rich | 2039 |
| | coiled-coil protein 2 | |
| Q13496_C53 | MTM1 Myotubularin | 2040 |
| O15460_C510 | P4HA2 Prolyl 4-hydroxylase | 2041 |
| | subunit alpha-2 | |
| P00558_C99 | PGK1 Phosphoglycerate | 2042 |
| | kinase 1 | |
| P05412_C99 | JUN Transcription factor AP-1 | 2043 |
| Q9H3R5_C35 | CENPH Centromere protein H | 2044 |
| Q02543_C16 | RPL18A 60S ribosomal | 2045 |
| | protein L18a | |
| Q06587_C69 | RING1 E3 ubiquitin-protein | 2046 |
| | ligase RING1 | |
| Q15365_C163 | PCBP1 Poly(rC)-binding | 2047 |
| | protein 1 | |
| O75369_C660 | FLNB Filamin-B | 2048 |
| O75369_C2537 | FLNB Filamin-B | 2049 |
| Q5RKV6_C117 | EXOSC6 Exosome complex | 2050 |
| | component MTR3 | |
| O15067_C606 | PFAS | 2051 |
| | Phosphoribosylformylglycinamidine | |
| | synthase | |
| Q9HB19_C191 | PLEKHA2 Pleckstrin | 2052 |
| | homology domain-containing | |
| | family A member 2 | |
| Q8WVV9_C84 | HNRPLL Heterogeneous | 2053 |
| | nuclear ribonucleoprotein L- | |
| | like | |
| O95400_C234 | CD2BP2 CD2 antigen | 2054 |
| | cytoplasmic tail-binding | |
| | protein 2 | |
| O94804_C947 | STK10 Serine/threonine- | 2055 |
| | protein kinase 10 | |
| Q5TBB1_C56 | RNASEH2B Ribonuclease H2 | 2056 |
| | subunit B | |
| Q96GW9_C425 | MARS2 Methionine--tRNA | 2057 |
| | ligase, mitochondrial | |
| P82912_C112 | MRPS11 28S ribosomal | 2058 |
| | protein S11, mitochondrial | |
| Q16643_C613 | DBN1 Drebrin | 2059 |
| Q9UPQ0_C577 | LIMCH1 LIM and calponin | 2060 |
| | homology domains-containing | |
| | prote | |
| P21333_C1453 | FLNA Filamin-A | 2061 |
| Q9UGV2_C166 | NDRG3 Protein NDRG3 | 2062 |
| P40926_C89 | MDH2 Malate dehydrogenase, | 2063 |
| | mitochondrial | |
| P40926_C93 | MDH2 Malate dehydrogenase, | 2064 |
| | mitochondrial | |
| Q14839_C1594 | CHD4 Chromodomain- | 2065 |
| | helicase-DNA-binding protein | |
| | 4 | |
| O95273_C300 | CCNDBP1 Cyclin-D1-binding | 2066 |
| | protein 1 | |
| A6NE09_C163 | RPSAP58 Protein RPSAP58 | 2067 |
| P62942_C23 | FKBP1A Peptidyl-prolyl cis- | 2068 |
| | trans isomerase FKBP1A | |
| Q04446_C221 | GBE1 1,4-alpha-glucan- | 2069 |
| | branching enzyme | |
| P16219_C289 | ACADS Short-chain specific | 2070 |
| | acyl-CoA dehydrogenase, | |
| | mitochondrial | |
| Q9Y3C6_C133 | PPIL1 Peptidyl-prolyl cis-trans | 2071 |
| | isomerase-like 1 | |
| P61970_C80 | NUTF2 Nuclear transport | 2072 |
| | factor 2 | |
| O14776_C535 | TCERG1 Transcription | 2073 |
| | elongation regulator 1 | |
| Q9BRP1_C278 | PDCD2L Programmed cell | 2074 |
| | death protein 2-like | |
| P13639_C567 | EEF2 Elongation factor 2 | 2075 |
| Q8WUX2_C113 | CHAC2 Cation transport | 2076 |
| | regulator-like protein 2 | |
| Q9H8W4_C219 | PLEKHF2 Pleckstrin | 2077 |
| | homology domain-containing | |
| | family F member 2 | |
| Q15021_C286 | NCAPD2 Condensin complex | 2078 |
| | subunit 1 | |
| O95394_C93 | PGM3 | 2079 |
| | Phosphoacetylglucosamine | |
| | mutase | |
| Q9NP61_C312 | ARFGAP3 ADP-ribosylation | 2080 |
| | factor GTPase-activating | |
| | protein | |
| Q86TG7_C119 | PEG10 Retrotransposon- | 2081 |
| | derived protein PEG10 | |
| Q7L0Y3_C78 | TRMT10C Mitochondrial | 2082 |
| | ribonuclease P protein 1 | |
| P35579_C694 | MYH9 Myosin-9 | 2083 |
| Q7Z7A4_C570 | PXK PX domain-containing | 2084 |
| | protein kinase-like protein | |
| P05091_C386 | ALDH2 Aldehyde | 2085 |
| | dehydrogenase, mitochondrial | |
| O15231_C615 | ZNF185 Zinc finger protein | 2086 |
| | 185 | |
| Q9P2J5_C573 | LARS Leucine--tRNA ligase, | 2087 |
| | cytoplasmic | |
| P41250_C525 | GARS Glycine--tRNA ligase | 2088 |
| Q9NR33_C84 | POLE4 DNA polymerase | 2089 |
| | epsilon subunit 4 | |
| Q9NQW6_C210 | ANLN Actin-binding protein | 2090 |
| | anillin | |
| Q9NQW6_C1117 | ANLN Actin-binding protein | 2091 |
| | anillin | |
| Q9HCC0_C267 | MCCC2 Methylcrotonoyl-CoA | 2092 |
| | carboxylase beta chain, mitoch | |
| P55884_C420 | EIF3B Eukaryotic translation | 2093 |
| | initiation factor 3 subunit | |
| P20290_C22 | BTF3 Transcription factor | 2094 |
| | BTF3 | |
| P49368_C398 | CCT3 T-complex protein 1 | 2095 |
| | subunit gamma | |
| O75600_C106 | GCAT 2-amino-3-ketobutyrate | 2096 |
| | coenzyme A ligase, | |
| | mitochondrial | |
| Q6PKG0_C864 | LARP1 La-related protein 1 | 2097 |
| Q5H9R7_C467 | PPP6R3 Serine/threonine- | 2098 |
| | protein phosphatase 6 | |
| | regulatory | |
| Q9H4A4_C311 | RNPEP Aminopeptidase B | 2099 |
| Q9BUF5_C12 | TUBB6 Tubulin beta-6 chain | 2100 |
| P52272_C676 | HNRNPM Heterogeneous | 2101 |
| | nuclear ribonucleoprotein M | |
| Q8TB03_C300 | CXorf38 Uncharacterized | 2102 |
| | protein CXorf38 | |
| Q9H7D7_C656 | WDR26 WD repeat-containing | 2103 |
| | protein 26 | |
| P50395_C317 | GDI2 Rab GDP dissociation | 2104 |
| | inhibitor beta | |
| P25205_C263 | MCM3 DNA replication | 2105 |
| | licensing factor MCM3 | |
| Q8TD19_C878 | NEK9 Serine/threonine-protein | 2106 |
| | kinase Nek9 | |
| P31947_C96 | SFN 14-3-3 protein sigma | 2107 |
| P49321_C568 | NASP Nuclear autoantigenic | 2108 |
| | sperm protein | |
| Q7L2J0_C54 | MEPCE 7SK snRNA | 2109 |
| | methylphosphate capping | |
| | enzyme | |
| Q96BN8_C347 | FAM105B Protein FAM105B | 2110 |
| Q12888_C1159 | TP53BP1 Tumor suppressor | 2111 |
| | p53-binding protein 1 | |
| P29034_C22 | S100A2 Protein S100-A2 | 2112 |
| P61978_C205 | HNRNPK Heterogeneous | 2113 |
| | nuclear ribonucleoprotein K | |
| P78527_C3837 | PRKDC DNA-dependent | 2114 |
| | protein kinase catalytic subunit | |
| Q7LG56_C279 | RRM2B Ribonucleoside- | 2115 |
| | diphosphate reductase subunit | |
| | M2 B | |
| P12814_C180 | ACTN1 Alpha-actinin-1 | 2116 |
| Q9ULV4_C343 | CORO1C Coronin-1C | 2117 |
| P22234_C185 | PAICS Multifunctional protein | 2118 |
| | ADE2 | |
| P22234_C288 | PAICS Multifunctional protein | 2119 |
| | ADE2 | |
| P30837_C386 | ALDH1B1 Aldehyde | 2120 |
| | dehydrogenase X, | |
| | mitochondrial | |
| P25098_C120 | ADRBK1 Beta-adrenergic | 2121 |
| | receptor kinase 1 | |
| P35998_C180 | PSMC2 26S protease | 2122 |
| | regulatory subunit 7 | |
| P08243_C255 | ASNS Asparagine synthetase | 2123 |
| O95819_C202 | MAP4K4 Mitogen-activated | 2124 |
| | protein kinase kinase kinase | |
| | kin | |
| Q13557_C273 | CAMK2D | 2125 |
| | Calcium/calmodulin-dependent | |
| | protein kinase type I | |
| P50213_C127 | IDH3A Isocitrate | 2126 |
| | dehydrogenase | |
| O95336_C237 | PGLS 6- | 2127 |
| | phosphogluconolactonase | |
| Q13155_C143 | AIMP2 Aminoacyl tRNA | 2128 |
| | synthase complex-interacting | |
| | multifunctional protein 2 | |
| Q14204_C3940 | DYNC1H1 Cytoplasmic | 2129 |
| | dynein 1 heavy chain 1 | |
| P30154_C306 | PPP2R1B Serine/threonine- | 2130 |
| | protein phosphatase 2A 65 kDa | |
| | regulatory subunit A beta | |
| | isoform | |
| P61758_C113 | VBP1 Prefoldin subunit 3 | 2131 |
| P48739_C13 | PITPNB Phosphatidylinositol | 2132 |
| | transfer protein beta isoform | |
| P08134_C159 | RHOC Rho-related GTP- | 2133 |
| | binding protein RhoC | |
| P15121_C81 | AKR1B1 Aldose reductase | 2134 |
| P86790_C358 | CCZ1B Vacuolar fusion | 2135 |
| | protein CCZ1 homolog B | |
| P42704_C863 | LRPPRC Leucine-rich PPR | 2136 |
| | motif-containing protein, | |
| | mitochondrial | |
| P33316_C166 | DUT Deoxyuridine 5- | 2137 |
| | triphosphate | |
| | nucleotidohydrolase, | |
| Q92597_C289 | NDRG1 Protein NDRG1 | 2138 |
| P35236_C62 | PTPN7 Tyrosine-protein | 2139 |
| | phosphatase non-receptor type | |
| | 7 | |
| P17987_C296 | TCP1 T-complex protein 1 | 2140 |
| | subunit alpha | |
| Q6PI48_C590 | DARS2 Aspartate--tRNA | 2141 |
| | ligase, mitochondrial | |
| P63244_C249 | GNB2L1 Guanine nucleotide- | 2142 |
| | binding protein subunit beta-2- | |
| | like 1 | |
| Q12874_C437 | SF3A3 Splicing factor 3A | 2143 |
| | subunit 3 | |
| Q9Y6W5_C27 | WASF2 Wiskott-Aldrich | 2144 |
| | syndrome protein family | |
| | member 2 | |
| P04075_C290 | ALDOA Fructose- | 2145 |
| | bisphosphate aldolase A | |
| Q8WW33_C76 | GTSF1 Gametocyte-specific | 2146 |
| | factor 1 | |
| P57081_C137 | WDR4 tRNA (guanine-N(7)-)- | 2147 |
| | methyltransferase subunit | |
| | WDR | |
| O60343_C1277 | TBC1D4 TBC1 domain family | 2148 |
| | member 4 | |
| P30084_C111 | ECHS1 Enoyl-CoA hydratase, | 2149 |
| | mitochondrial | |
| P12268_C173 | IMPDH2 Inosine-5- | 2150 |
| | monophosphate dehydrogenase | |
| | 2 | |
| O43290_C674 | SART1 U4/U6.U5 tri-snRNP- | 2151 |
| | associated protein 1 | |
| Q9P258_C280 | RCC2 Protein RCC2 | 2152 |
| P15153_C6 | RAC2 Ras-related C3 | 2153 |
| | botulinum toxin substrate 2 | |
| O60361_C94 | NME2P1 Putative nucleoside | 2154 |
| | diphosphate kinase | |
| O75600_C219 | GCAT 2-amino-3-ketobutyrate | 2155 |
| | coenzyme A ligase, | |
| | mitochondrial | |
| O60568_C494 | PLOD3 Procollagen-lysine,2- | 2156 |
| | oxoglutarate 5-dioxygenase 3 | |
| Q9H0R6_C512 | QRSL1 Glutamyl-tRNA(Gln) | 2157 |
| | amidotransferase subunit A, | |
| | mitochondrial | |
| Q7RTV0_C11 | PHF5A PHD finger-like | 2158 |
| | domain-containing protein 5A | |
| Q96I99_C255 | SUCLG2 Succinyl-CoA ligase | 2159 |
| A0AVT1_C770 | UBA6 Ubiquitin-like modifier- | 2160 |
| | activating enzyme 6 | |
| P08047_C755 | SP1 Transcription factor Sp1 | 2161 |
| P26641_C166 | EEF1G Elongation factor 1- | 2162 |
| | gamma | |
| Q16270_C131 | IGFBP7 Insulin-like growth | 2163 |
| | factor-binding protein 7 | |
| Q9BQ52_C51 | ELAC2 Zinc | 2164 |
| | phosphodiesterase ELAC | |
| | protein 2 | |
| Q6YN16_C11 | HSDL2 Hydroxysteroid | 2165 |
| | dehydrogenase-like protein 2 | |
| Q9BQP7_C79 | C20orf72 Uncharacterized | 2166 |
| | protein C20orf72 | |
| Q7Z6Z7_C2865 | HUWE1 E3 ubiquitin-protein | 2167 |
| | ligase HUWE1 | |
| Q7Z6Z7_C3259 | HUWE1 E3 ubiquitin-protein | 2168 |
| | ligase HUWE1 | |
| Q7Z6Z7_C3658 | HUWE1 E3 ubiquitin-protein | 2169 |
| | ligase HUWE1 | |
| Q14914_C239 | PTGR1 Prostaglandin | 2170 |
| | reductase 1 | |
| O00268_C1022 | TAF4 Transcription initiation | 2171 |
| | factor TFIID subunit 4 | |
| O95340_C155 | PAPSS2 Bifunctional 3- | 2172 |
| | phosphoadenosine 5- | |
| | phosphosulfate | |
| O95295_C66 | SNAPIN SNARE-associated | 2173 |
| | protein Snapin | |
| P43490_C287 | NAMPT Nicotinamide | 2174 |
| | phosphoribosyltransferase | |
| P49327_C135 | FASN Fatty acid synthase | 2175 |
| Q9Y383_C59 | LUC7L2 Putative RNA- | 2176 |
| | binding protein Luc7-like 2 | |
| Q9NS86_C49 | LANCL2 LanC-like protein 2 | 2177 |
| Q15149_C317 | PLEC Plectin | 2178 |
| O60825_C430 | PFKFB2 6-phosphofructo-2- | 2179 |
| | kinase/fructose-2,6- | |
| | bisphosphatase 2 | |
| Q5JS54_C55 | PSMG4 Proteasome assembly | 2180 |
| | chaperone 4 | |
| P52566_C76 | ARHGDIB Rho GDP- | 2181 |
| | dissociation inhibitor 2 | |
| P07203_C156 | GPX1 Glutathione peroxidase | 2182 |
| | 1 | |
| Q9UL25_C29 | RAB21 Ras-related protein | 2183 |
| | Rab-21 | |
| P49841_C14 | GSK3B Glycogen synthase | 2184 |
| | kinase-3 beta | |
| P17655_C39 | CAPN2 Calpain-2 catalytic | 2185 |
| | subunit | |
| Q14315_C2679 | FLNC Filamin-C | 2186 |
| P32969_C74 | RPL9P9 60S ribosomal protein | 2187 |
| | L9 | |
| Q96JH7_C1178 | VCPIP1 Deubiquitinating | 2188 |
| | protein VCIP135 | |
| Q9NP61_C241 | ARFGAP3 ADP-ribosylation | 2189 |
| | factor GTPase-activating | |
| | protein | |
| Q9Y265_C141 | RUVBL1 RuvB-like 1 | 2190 |
| P52789_C158 | HK2 Hexokinase-2 | 2191 |
| P35579_C172 | MYH9 Myosin-9 | 2192 |
| P12955_C482 | PEPD Xaa-Pro dipeptidase | 2193 |
| Q86UX7_C235 | FERMT3 Fermitin family | 2194 |
| | homolog 3 | |
| Q15003_C239 | NCAPH Condensin complex | 2195 |
| | subunit 2 | |
| P07814_C1377 | EPRS Bifunctional | 2196 |
| | glutamate/proline--tRNA | |
| | ligase | |
| P11586_C143 | MTHFD1 C-1-tetrahydrofolate | 2197 |
| | synthase, cytoplasmic | |
| P62312_C36 | LSM6 U6 snRNA-associated | 2198 |
| | Sm-like protein LSm6 | |
| P55209_C132 | NAP1L1 Nucleosome | 2199 |
| | assembly protein 1-like 1 | |
| O75815_C699 | BCAR3 Breast cancer anti- | 2200 |
| | estrogen resistance protein 3 | |
| P24534_C191 | EEF1B2 Elongation factor 1- | 2201 |
| | beta | |
| P15121_C200 | AKR1B1 Aldose reductase | 2202 |
| Q9GZU8_C187 | FAM192A Protein FAM192A | 2203 |
| P25789_C34 | PSMA4 Proteasome subunit | 2204 |
| | alpha type-4 | |
| P25789_C74 | PSMA4 Proteasome subunit | 2205 |
| | alpha type-4 | |
| P07355_C133 | ANXA2 Annexin A2 | 2206 |
| Q15393_C410 | SF3B3 Splicing factor 3B | 2207 |
| | subunit 3 | |
| P63244_C240 | GNB2L1 Guanine nucleotide- | 2208 |
| | binding protein subunit beta-2- | |
| | like 1 | |
| P61247_C111 | RPS3A 40S ribosomal protein | 2209 |
| | S3a | |
| P53582_C40 | METAP1 Methionine | 2210 |
| | aminopeptidase 1 | |
| P23921_C790 | RRM1 Ribonucleoside- | 2211 |
| | diphosphate reductase large | |
| | subunit R | |
| Q9BT78_C378 | COPS4 COP9 signalosome | 2212 |
| | complex subunit 4 | |
| Q7KZ85_C336 | SUPT6H Transcription | 2213 |
| | elongation factor SPT6 | |
| P23381_C274 | WARS Tryptophan--tRNA | 2214 |
| | ligase, cytoplasmic | |
| P36551_C239 | CPOX Coproporphyrinogen- | 2215 |
| | III oxidase, mitochondrial | |
| Q7KZF4_C31 | SND1 Staphylococcal nuclease | 2216 |
| | domain-containing protein | |
| Q14139_C1002 | UBE4A Ubiquitin conjugation | 2217 |
| | factor E4 A | |
| Q8NCF5_C232 | NFATC2IP NFATC2- | 2218 |
| | interacting protein | |
| O75934_C106 | BCAS2 Pre-mRNA-splicing | 2219 |
| | factor SPF27 | |
| Q96IF1_C158 | AJUBA LIM domain- | 2220 |
| | containing protein ajuba | |
| Q9NZL4_C22 | HSPBP1 Hsp70-binding | 2221 |
| | protein 1 | |
| Q9NZL9_C58 | MAT2B Methionine | 2222 |
| | adenosyltransferase 2 subunit | |
| | beta | |
| Q5TDH0_C361 | DDI2 Protein DDI1 homolog 2 | 2223 |
| Q13126_C131 | MTAP S-methyl-5- | 2224 |
| | thioadenosine phosphorylase | |
| O75369_C706 | FLNB Filamin-B | 2225 |
| P21980_C269 | TGM2 Protein-glutamine | 2226 |
| | gamma-glutamyltransferase 2 | |
| Q16270_C113 | IGFBP7 Insulin-like growth | 2227 |
| | factor-binding protein 7 | |
| H7BZ11_C88 | Uncharacterized protein | 2228 |
| Q96FZ2_C131 | C3orf37 UPF0361 protein | 2229 |
| | C3orf37 | |
| Q6YN16_C71 | HSDL2 Hydroxysteroid | 2230 |
| | dehydrogenase-like protein 2 | |
| Q9UBL3_C581 | ASH2L Set1/Ash2 histone | 2231 |
| | methyltransferase complex | |
| | subunit | |
| Q15370_C60 | TCEB2 Transcription | 2232 |
| | elongation factor B | |
| | polypeptide 2 | |
| O43252_C360 | PAPSS1 Bifunctional 3- | 2233 |
| | phosphoadenosine 5- | |
| | phosphosulfete | |
| P62633_C171 | CNBP Cellular nucleic acid- | 2234 |
| | binding protein | |
| P63151_C398 | PPP2R2A Serine/threonine- | 2235 |
| | protein phosphatase 2A 55 kDa | |
| | regulatory subunit B alpha | |
| | isoform | |
| Q16881_C209 | TXNRD1 Thioredoxin | 2236 |
| | reductase 1, cytoplasmic | |
| O95294_C761 | RASAL1 RasGAP-activating- | 2237 |
| | like protein 1 | |
| Q9UQ35_C872 | SRRM2 Serine/arginine | 2238 |
| | repetitive matrix protein 2 | |
| P78371_C535 | CCT2 T-complex protein 1 | 2239 |
| | subunit beta | |
| P54577_C250 | YARS Tyrosine--tRNA ligase, | 2240 |
| | cytoplasmic | |
| Q8TD30_C477 | GPT2 Alanine | 2241 |
| | aminotransferase 2 | |
| Q15149_C3299 | PLEC Plectin | 2242 |
| O14744_C278 | PRMT5 Protein arginine N- | 2243 |
| | methyltransferase 5 | |
| Q7L1Q6_C96 | BZW1 Basic leucine zipper | 2244 |
| | and W2 domain-containing | |
| | prot | |
| P04181_C150 | OAT Ornithine | 2245 |
| | aminotransferase, | |
| | mitochondrial | |
| P04181_C330 | OAT Ornithine | 2246 |
| | aminotransferase, | |
| | mitochondrial | |
| Q9NVT9_C69 | ARMC1 Armadillo repeat- | 2247 |
| | containing protein 1 | |
| O60888_C96 | CUTA Protein CutA | 2248 |
| Q6P2E9_C976 | EDC4 Enhancer of mRNA- | 2249 |
| | decapping protein 4 | |
| P20810_C408 | CAST Calpastatin | 2250 |
| Q5T6F2_C208 | UBAP2 Ubiquitin-associated | 2251 |
| | protein 2 | |
| Q9BQ61_C165 | C19orf43 Uncharacterized | 2252 |
| | protein C19orf43 | |
| P54687_C12 | BCAT1 Branched-chain- | 2253 |
| | amino-acid aminotransferase, | |
| | cytosolic | |
| P54687_C335 | BCAT1 Branched-chain- | 2254 |
| | amino-acid aminotransferase, | |
| | cytosolic | |
| Q96I15_C22 | SCLY Selenocysteine lyase | 2255 |
| P45974_C335 | USP5 Ubiquitin carboxyl- | 2256 |
| | terminal hydrolase 5 | |
| O14497_C336 | ARID1A AT-rich interactive | 2257 |
| | domain-containing protein 1A | |
| O43776_C438 | NARS Asparagine--tRNA | 2258 |
| | ligase, cytoplasmic | |
| P12955_C467 | PEPD Xaa-Pro dipeptidase | 2259 |
| Q9Y678_C97 | COPG1 Coatomer subunit | 2260 |
| | gamma-1 | |
| Q9Y678_C169 | COPG1 Coatomer subunit | 2261 |
| | gamma-1 | |
| P55786_C339 | NPEPPS Puromycin-sensitive | 2262 |
| | aminopeptidase | |
| Q9Y520_C223 | PRRC2C Protein PRRC2C | 2263 |
| P52306_C29 | RAP1GDS1 Rap1 GTPase- | 2264 |
| | GDP dissociation stimulator 1 | |
| Q6GMV2_C194 | SMYD5 SET and MYND | 2265 |
| | domain-containing protein 5 | |
| Q16763_C95 | UBE2S Ubiquitin-conjugating | 2266 |
| | enzyme E2 S | |
| Q7L014_C590 | DDX46 Probable ATP- | 2267 |
| | dependent RNA helicase | |
| | DDX46 | |
| Q8NFX7_C144 | STXBP6 Syntaxin-binding | 2268 |
| | protein 6 | |
| P56537_C11 | EIF6 Eukaryotic translation | 2269 |
| | initiation factor 6 | |
| Q9NRL3_C17 | STRN4 Striatin-4 | 2270 |
| P41252_C185 | IARS Isoleucine--tRNA ligase, | 2271 |
| | cytoplasmic | |
| O60343_C45 | TBC1D4 TBC1 domain family | 2272 |
| | member 4 | |
| P30086_C133 | PEBP1 | 2273 |
| | Phosphatidylethanolamine- | |
| | binding protein 1 | |
| Q9NQW7_C308 | XPNPEP1 Xaa-Pro | 2274 |
| | aminopeptidase 1 | |
| O00299_C89 | CLIC1 Chloride intracellular | 2275 |
| | channel protein 1 | |
| Q9Y4B6_C784 | VPRBP Protein VPRBP | 2276 |
| Q66PJ3_C349 | ARL6IP4 ADP-ribosylation | 2277 |
| | factor-like protein 6-interacting | |
| P00367_C327 | GLUD1 Glutamate | 2278 |
| | dehydrogenase 1, | |
| | mitochondrial | |
| Q14C86_C568 | GAPVD1 GTPase-activating | 2279 |
| | protein and VPS9 domain- | |
| | containing protein 1 | |
| P09382_C89 | LGALS1 Galectin-1 | 2280 |
| O00244_C41 | ATOX1 Copper transport | 2281 |
| | protein ATOX1 | |
| P16885_C624 | PLCG2 1-phosphatidylinositol | 2282 |
| | 4,5-bisphosphate | |
| | phosphodiesterase | |
| Q92835_C138 | INPP5D Phosphatidylinositol | 2283 |
| | 3,4,5-trisphosphate 5- | |
| | phosphatase 1 | |
| J3QR44_C430 | CDK11B Cyclin-dependent | 2284 |
| | kinase 11B | |
| Q9P289_C352 | MST4 Serine/threonine-protein | 2285 |
| | kinase MST4 | |
| Q92688_C27 | ANP32B Acidic leucine-rich | 2286 |
| | nuclear phosphoprotein 32 | |
| | family member B | |
| Q96GX9_C97 | APIP Probable | 2287 |
| | methylthioribulose-1- | |
| | phosphate dehydratase | |
| Q15004_C54 | KIAA0101 PCNA-associated | 2288 |
| | factor | |
| O75083_C325 | WDR1 WD repeat-containing | 2289 |
| | protein 1 | |
| O43252_C207 | PAPSS1 Bifunctional 3- | 2290 |
| | phosphoadenosine 5- | |
| | phosphosulfate | |
| P62633_C161 | CNBP Cellular nucleic acid- | 2291 |
| | binding protein | |
| Q7Z6Z7_C2832 | HUWE1 E3 ubiquitin-protein | 2292 |
| | ligase HUWE1 | |
| Q8NCW5_C91 | APOA1BP NAD(P)H-hydrate | 2293 |
| | epimerase | |
| P31949_C13 | S100A11 Protein S100-A11 | 2294 |
| P07900_C572 | HSP90AA1 Heat shock protein | 2295 |
| | HSP 90-alpha | |
| Q86UA6_C122 | RPAIN RPA-interacting | 2296 |
| | protein | |
| Q8N6M0_C172 | OTUD6B OTU domain- | 2297 |
| | containing protein 6B | |
| Q13153_C411 | PAK1 Serine/threonine-protein | 2298 |
| | kinase PAK 1 | |
| O60232_C53 | SSSCA1 Sjoegren | 2299 |
| | syndrome/scleroderma | |
| | autoantigen 1 | |
| Q9NQY0_C72 | BIN3 Bridging integrator 3 | 2300 |
| O60942_C419 | RNGTT mRNA-capping | 2301 |
| | enzyme | |
| P13804_C155 | ETFA Electron transfer | 2302 |
| | flavoprotein subunit alpha, | |
| | mitochondrial | |
| Q96QR8_C17 | PURB Transcriptional | 2303 |
| | activator protein Pur-beta | |
| O60711_C199 | LPXN Leupaxin | 2304 |
| Q00013_C242 | MPP1 55 kDa erythrocyte | 2305 |
| | membrane protein | |
| P52597_C290 | HNRNPF Heterogeneous | 2306 |
| | nuclear ribonucleoprotein F | |
| P09211_C102 | GSTP1 Glutathione S- | 2307 |
| | transferase P | |
| Q15149_C950 | PLEC Plectin | 2308 |
| P62195_C209 | PSMC5 26S protease | 2309 |
| | regulatory subunit 8 | |
| Q9Y2Q3_C176 | GSTK1 Glutathione S- | 2310 |
| | transferase kappa 1 | |
| O15164_C629 | TRIM24 Transcription | 2311 |
| | intermediary factor 1-alpha | |
| P22314_C234 | UBA1 Ubiquitin-like modifier- | 2312 |
| | activating enzyme 1 | |
| Q96HE7_C94 | ERO1L ERO1-like protein | 2313 |
| | alpha | |
| P07203_C78 | GPX1 Glutathione peroxidase | 2314 |
| | 1 | |
| P21964_C238 | COMT Catechol O- | 2315 |
| | methyltransferase | |
| O76071_C72 | CIAO1 Probable cytosolic | 2316 |
| | iron-sulfur protein assembly | |
| | protein CIAO1 | |
| P04083_C324 | ANXA1 Annexin A1 | 2317 |
| Q14315_C1109 | FLNC Filamin-C | 2318 |
| Q13526_C57 | PIN1 Peptidyl-prolyl cis-trans | 2319 |
| | isomerase NIMA-interacting 1 | |
| Q9Y3B2_C170 | EXOSC1 Exosome complex | 2320 |
| | component CSL4 | |
| P54687_C140 | BCAT1 Branched-chain- | 2321 |
| | amino-acid aminotransferase, | |
| | cytosolic | |
| Q92945_C379 | KHSRP Far upstream element- | 2322 |
| | binding protein 2 | |
| Q13148_C198 | TARDBP TAR DNA-binding | 2323 |
| | protein 43 | |
| Q9Y3C4_C167 | TPRKB TP53RK-binding | 2324 |
| | protein | |
| O43175_C281 | PHGDH D-3- | 2325 |
| | phosphoglycerate | |
| | dehydrogenase | |
| Q16698_C285 | DECR1 2,4-dienoyl-CoA | 2326 |
| | reductase, mitochondrial | |
| P49773_C38 | HINT1 Histidine triad | 2327 |
| | nucleotide-binding protein 1 | |
| P12004_C27 | PCNA Proliferating cell | 2328 |
| | nuclear antigen | |
| P51553_C81 | IDH3G Isocitrate | 2329 |
| | dehydrogenase | |
| Q14192_C214 | FHL2 Four and a half LIM | 2330 |
| | domains protein 2 | |
| P07814_C381 | EPRS Bifunctional | 2331 |
| | glutamate/proline--tRNA | |
| | ligase | |
| Q8WWY3_C247 | PRPF31 U4/U6 small nuclear | 2332 |
| | ribonucleoprotein Prp31 | |
| Q13085_C813 | ACACA Acetyl-CoA | 2333 |
| | carboxylase 1 | |
| O43765_C38 | SGTA Small glutamine-rich | 2334 |
| | tetratricopeptide repeat-cont | |
| Q13185_C160 | CBX3 Chromobox protein | 2335 |
| | homolog 3 | |
| Q9UBR2_C164 | CTSZ Cathepsin Z | 2336 |
| P54886_C612 | ALDH18A1 Delta-1-pyrroline- | 2337 |
| | 5-carboxylate synthase | |
| Q9H9E3_C494 | COG4 Conserved oligomeric | 2338 |
| | Golgi complex subunit 4 | |
| P19623_C209 | SRM Spermidine synthase | 2339 |
| P61247_C201 | RPS3A 40S ribosomal protein | 2340 |
| | S3a | |
| P15311_C284 | EZR Ezrin | 2341 |
| P53004_C204 | BLVRA Biliverdin reductase | 2342 |
| | A | |
| Q969E8_C114 | TSR2 Pre-rRNA-processing | 2343 |
| | protein TSR2 homolog | |
| P41252_C350 | IARS Isoleucine--tRNA ligase, | 2344 |
| | cytoplasmic | |
| O60343_C316 | TBC1D4 TBC1 domain family | 2345 |
| | member 4 | |
| Q96JB2_C349 | COG3 Conserved oligomeric | 2346 |
| | Golgi complex subunit 3 | |
| Q9Y490_C1978 | TLN1 Talin-1 | 2347 |
| P23381_C62 | WARS Tryptophan--tRNA | 2348 |
| | ligase, cytoplasmic | |
| P27708_C280 | CAD CAD protein | 2349 |
| Q9Y3Z3_C80 | SAMHD1 SAM domain and | 2350 |
| | HD domain-containing protein | |
| | 1 | |
| Q53H96_C49 | PYCRL Pyrroline-5- | 2351 |
| | carboxylate reductase 3 | |
| Q9HCC0_C431 | MCCC2 Methylcrotonoyl-CoA | 2352 |
| | carboxylase beta chain, | |
| | mitochondrial | |
| P47897_C358 | QARS Glutamine--tRNA | 2353 |
| | ligase | |
| P55084_C435 | HADHB Trifunctional enzyme | 2354 |
| | subunit beta, mitochondrial | |
| P30405_C157 | PPIF Peptidyl-prolyl cis-trans | 2355 |
| | isomerase F, mitochondrial | |
| P08567_C160 | PLEK Pleckstrin | 2356 |
| A6NHR9_C84 | SMCHD1 Structural | 2357 |
| | maintenance of chromosomes | |
| | flexible hinge domain | |
| | containing 1 | |
| Q9Y2X3_C439 | NOP58 Nucleolar protein 58 | 2358 |
| O00410_C420 | IPO5 Importin-5 | 2359 |
| P09382_C61 | LGALS1 Galectin-1 | 2360 |
| O75150_C950 | RNF40 E3 ubiquitin-protein | 2361 |
| | ligase BRE1B | |
| P11021_C420 | HSPA5 78 kDa glucose- | 2362 |
| | regulated protein | |
| P29401_C133 | TKT Transketolase | 2363 |
| Q92598_C658 | HSPH1 Heat shock protein 105 | 2364 |
| | kDa | |
| O43795_C129 | MYO1B Unconventional | 2365 |
| | myosin-Ib | |
| O75369_C1326 | FLNB Filamin-B | 2366 |
| Q70E73_C94 | RAPH1 Ras-associated and | 2367 |
| | pleckstrin homology domains- | |
| | con | |
| Q0PNE2_C218 | ELP6 Elongator complex | 2368 |
| | protein 6 | |
| Q12996_C628 | CSTF3 Cleavage stimulation | 2369 |
| | factor subunit 3 | |
| P09543_C49 | CNP 2,3-cyclic-nucleotide 3- | 2370 |
| | phosphodiesterase | |
| Q8WVV9_C405 | HNRPLL Heterogeneous | 2371 |
| | nuclear ribonucleoprotein L- | |
| | like | |
| Q8N4X5_C467 | AFAP1L2 Actin filament- | 2372 |
| | associated protein 1-like 2 | |
| Q8IUI8_C313 | CRLF3 Cytokine receptor-like | 2373 |
| | factor 3 | |
| Q9BPX3_C843 | NCAPG Condensin complex | 2374 |
| | subunit 3 | |
| P30566_C27 | ADSL Adenylosuccinate lyase | 2375 |
| O00469_C494 | PLOD2 Procollagen-lysine,2- | 2376 |
| | oxoglutarate 5-dioxygenase 2 | |
| Q92616_C55 | GCN1L1 Translational | 2377 |
| | activator GCN1 | |
| P49327_C212 | FASN Fatty acid synthase | 2378 |
| P42166_C330 | TMPO Lamina-associated | 2379 |
| | polypeptide 2, isoform alpha | |
| P55769_C73 | NHP2L1 NHP2-like protein 1 | 2380 |
| Q9UQ35_C2116 | SRRM2 Serine/arginine | 2381 |
| | repetitive matrix protein 2 | |
| P01591_C91 | IGJ Immunoglobulin J chain | 2382 |
| P61970_C38 | NUTF2 Nuclear transport | 2383 |
| | factor 2 | |
| O94953_C547 | KDM4B Lysine-specific | 2384 |
| | demethylase 4B | |
| Q15149_C730 | PLEC Plectin | 2385 |
| Q9ULW0_C301 | TPX2 Targeting protein for | 2386 |
| | Xklp2 | |
| Q9UNF1_C516 | MAGED2 Melanoma- | 2387 |
| | associated antigen D2 | |
| P51808_C8 | DYNLT3 Dynein light chain | 2388 |
| | Tctex-type 3 | |
| Q09666_C5502 | AHNAK Neuroblast | 2389 |
| | differentiation-associated | |
| | protein AHNA | |
| P27348_C25 | YWHAQ 14-3-3 protein theta | 2390 |
| Q8WUM0_C811 | NUP133 Nuclear pore | 2391 |
| | complex protein Nup133 | |
| P52789_C628 | HK2 Hexokinase-2 | 2392 |
| O60716_C618 | CTNND1 Catenin delta-1 | 2393 |
| P26599_C251 | PTBP1 Polypyrimidine tract- | 2394 |
| | binding protein 1 | |
| P23528_C147 | CFL1 Cofilin-1 | 2395 |
| Q14694_C94 | USP10 Ubiquitin carboxyl- | 2396 |
| | terminal hydrolase 10 | |
| P36507_C384 | MAP2K2 Dual specificity | 2397 |
| | mitogen-activated protein | |
| | kinase | |
| Q9UJX3_C259 | ANAPC7 Anaphase-promoting | 2398 |
| | complex subunit 7 | |
| Q9BV20_C168 | MRI1 Methylthioribose-1- | 2399 |
| | phosphate isomerase | |
| Q9Y696_C189 | CLIC4 Chloride intracellular | 2400 |
| | channel protein 4 | |
| Q6ZMK1_C342 | CYHR1 Cysteine and | 2401 |
| | histidine-rich protein 1 | |
| P45954_C139 | ACADSB Short/branched | 2402 |
| | chain specific acyl-CoA | |
| | dehydrogena | |
| P15121_C187 | AKR1B1 Aldose reductase | 2403 |
| Q9NR50_C64 | EIF2B3 Translation initiation | 2404 |
| | factor eIF-2B subunit gamma | |
| Q14980_C1907 | NUMA1 Nuclear mitotic | 2405 |
| | apparatus protein 1 | |
| O43707_C793 | ACTN4 Alpha-actinin-4 | 2406 |
| O43707_C879 | ACTN4 Alpha-actinin-4 | 2407 |
| Q9NPA3_C62 | MID1IP1 Mid1-interacting | 2408 |
| | protein 1 | |
| Q5UIP0_C2450 | RIF1 Telomere-associated | 2409 |
| | protein RIF1 | |
| P41134_C17 | ID1 DNA-binding protein | 2410 |
| | inhibitor ID-1 | |
| Q13509_C129 | TUBB3 Tubulin beta-3 chain | 2411 |
| Q16718_C17 | NDUFA5 NADH | 2412 |
| | dehydrogenase | |
| Q9H6K1_C18 | C6orf106 Uncharacterized | 2413 |
| | protein C6orf106 | |
| Q13057_C144 | COASY Bifunctional | 2414 |
| | coenzyme A synthase | |
| P28838_C313 | LAP3 Cytosol aminopeptidase | 2415 |
| Q96EP0_C504 | RNF31 E3 ubiquitin-protein | 2416 |
| | ligase RNF31 | |
| P08107_C17 | HSPA1B Heat shock 70 kDa | 2417 |
| | protein 1A/1B | |
| P27708_C1455 | CAD CAD protein | 2418 |
| P23497_C309 | SP100 Nuclear autoantigen Sp- | 2419 |
| | 100 | |
| P11498_C739 | PC Pyruvate carboxylase, | 2420 |
| | mitochondrial | |
| Q02790_C396 | FKBP4 Peptidyl-prolyl cis- | 2421 |
| | trans isomerase FKBP4 | |
| P48507_C35 | GCLM Glutamate--cysteine | 2422 |
| | ligase regulatory subunit | |
| P55735_C245 | SEC13 Protein SEC13 | 2423 |
| | homolog | |
| P00367_C376 | GLUD1 Glutamate | 2424 |
| | dehydrogenase 1, | |
| | mitochondrial | |
| P52701_C1075 | MSH6 DNA mismatch repair | 2425 |
| | protein Msh6 | |
| Q14166_C612 | TTLL12 Tubulin--tyrosine | 2426 |
| | ligase-like protein 12 | |
| Q6PKG0_C502 | LARP1 La-related protein 1 | 2427 |
| Q9NVS9_C156 | PNPO Pyridoxine-5-phosphate | 2428 |
| | oxidase | |
| Q99873_C93 | PRMT1 Protein arginine N- | 2429 |
| | methyltransferase 1 | |
| Q9H4A6_C84 | GOLPH3 Golgi | 2430 |
| | phosphoprotein 3 | |
| P78347_C34 | GTF2I General transcription | 2431 |
| | factor II-I | |
| Q96FZ2_C39 | C3orf37 UPF0361 protein | 2432 |
| | C3orf37 | |
| O60547_C336 | GMDS GDP-mannose 4,6 | 2433 |
| | dehydratase | |
| P62979_C149 | RPS27A Ubiquitin-40S | 2434 |
| | ribosomal protein S27a | |
| P52732_C911 | KIF11 Kinesin-like protein | 2435 |
| | KIF11 | |
| P40938_C32 | RFC3 Replication factor C | 2436 |
| | subunit 3 | |
| P31943_C34 | HNRNPH1 Heterogeneous | 2437 |
| | nuclear ribonucleoprotein H | |
| Q8N1F7_C422 | NUP93 Nuclear pore complex | 2438 |
| | protein Nup93 | |
| Q16643_C96 | DBN1 Drebrin | 2439 |
| P45880_C76 | VDAC2 Voltage-dependent | 2440 |
| | anion-selective channel protein | |
| Q9BYG5_C13 | PARD6B Partitioning | 2441 |
| | defective 6 homolog beta | |
| P42765_C128 | ACAA2 3-ketoacyl-CoA | 2442 |
| | thiolase, mitochondrial | |
| Q96HC4_C213 | PDLIM5 PDZ and LIM | 2443 |
| | domain protein 5 | |
| O15042_C320 | U2SURP U2 snRNP- | 2444 |
| | associated SURP motif- | |
| | containing protein | |
| Q96IY1_C224 | NSL1 Kinetochore-associated | 2445 |
| | protein NSL1 homolog | |
| P12270_C1068 | TPR Nucleoprotein TPR | 2446 |
| P01591_C156 | IGJ Immunoglobulin J chain | 2447 |
| P53992_C1083 | SEC24C Protein transport | 2448 |
| | protein Sec24C | |
| Q92973_C142 | TNPO1 Transportin-1 | 2449 |
| P32321_C60 | DCTD Deoxycytidylate | 2450 |
| | deaminase | |
| P62195_C112 | PSMC5 26S protease | 2451 |
| | regulatory subunit 8 | |
| P31146_C40 | CORO1A Coronin-1A | 2452 |
| P12814_C774 | ACTN1 Alpha-actinin-1 | 2453 |
| Q04726_C528 | TLE3 Transducin-like | 2454 |
| | enhancer protein 3 | |
| Q52LJ0_C216 | FAM98B Protein FAM98B | 2455 |
| P04083_C270 | ANXA1 Annexin A1 | 2456 |
| O94925_C203 | GLS Glutaminase kidney | 2457 |
| | isoform, mitochondrial | |
| Q9GZP4_C187 | PITHD1 PITH domain- | 2458 |
| | containing protein 1 | |
| P33764_C14 | S100A3 Protein S100-A3 | 2459 |
| Q9UKK9_C76 | NUDT5 ADP-sugar | 2460 |
| | pyrophosphatase | |
| P38117_C71 | ETFB Electron transfer | 2461 |
| | flavoprotein subunit beta | |
| O95817_C151 | BAG3 BAG family molecular | 2462 |
| | chaperone regulator 3 | |
| Q27J81_C898 | INF2 Inverted formin-2 | 2463 |
| Q9C0C2_C1443 | TNKS1BP1 182 kDa | 2464 |
| | tankyrase-1-binding protein | |
| Q9C0C9_C314 | UBE2O Ubiquitin-conjugating | 2465 |
| | enzyme E2 O | |
| P84090_C69 | ERH Enhancer of rudimentary | 2466 |
| | homolog | |
| Q8TDN6_C52 | BRIX1 Ribosome biogenesis | 2467 |
| | protein BRX1 homolog | |
| P42704_C484 | LRPPRC Leucine-rich PPR | 2468 |
| | motif-containing protein, | |
| | mitochondrial | |
| Q9NR56_C53 | MBNL1 Muscleblind-like | 2469 |
| | protein 1 | |
| P14678_C43 | SNRPB Small nuclear | 2470 |
| | ribonucleoprotein-associated | |
| | protein | |
| Q86VP6_C1134 | CAND1 Cullin-associated | 2471 |
| | NEDD8-dissociated protein 1 | |
| P14324_C333 | FDPS Farnesyl pyrophosphate | 2472 |
| | synthase | |
| P07237_C312 | P4HB Protein disulfide- | 2473 |
| | isomerase | |
| Q9BXJ9_C238 | NAA15 N-alpha- | 2474 |
| | acetyltransferase 15, NatA | |
| | auxiliary subunit | |
| Q99614_C28 | TTC1 Tetratricopeptide repeat | 2475 |
| | protein 1 | |
| Q9UDY2_C914 | TJP2 Tight junction protein | 2476 |
| | ZO-2 | |
| Q06210_C254 | GFPT1 Glucosamine-- | 2477 |
| | fructose-6-phosphate | |
| | aminotransferase | |
| P55199_C14 | ELL RNA polymerase II | 2478 |
| | elongation factor ELL | |
| Q9H2U2_C180 | PPA2 Inorganic | 2479 |
| | pyrophosphatase 2, | |
| | mitochondrial | |
| Q6UB35_C779 | MTHFD1L Monofunctional | 2480 |
| | C1-tetrahydrofolate synthase, | |
| | mitochondrial | |
| P34897_C80 | SHMT2 Serine | 2481 |
| | hydroxymethyltransferase, | |
| | mitochondrial | |
| P13798_C30 | APEH Acylamino-acid- | 2482 |
| | releasing enzyme | |
| O95671_C333 | ASMTL N-acetylserotonin O- | 2483 |
| | methyltransferase-like protein | |
| O95671_C441 | ASMTL N-acetylserotonin O- | 2484 |
| | methyltransferase-like protein | |
| Q9NQW6_C309 | ANLN Actin-binding protein | 2485 |
| | anillin | |
| Q04206_C109 | RELA Transcription factor p65 | 2486 |
| P31350_C202 | RRM2 Ribonucleoside- | 2487 |
| | diphosphate reductase subunit | |
| | M2 | |
| P08107_C306 | HSPA1B Heat shock 70 kDa | 2488 |
| | protein 1A/1B | |
| O75914_C424 | PAK3 Serine/threonine-protein | 2489 |
| | kinase PAK 3 | |
| P27708_C379 | CAD CAD protein | 2490 |
| P62910_C96 | RPL32 60S ribosomal protein | 2491 |
| | L32 | |
| Q9Y570_C347 | PPME1 Protein phosphatase | 2492 |
| | methylesterase 1 | |
| Q92499_C110 | DDX1 ATP-dependent RNA | 2493 |
| | helicase DDX1 | |
| P30405_C203 | PPIF Peptidyl-prolyl cis-trans | 2494 |
| | isomerase F, mitochondrial | |
| Q92621_C1662 | NUP205 Nuclear pore | 2495 |
| | complex protein Nup205 | |
| Q14376_C196 | GALE UDP-glucose 4- | 2496 |
| | epimerase | |
| O75717_C1008 | WDHD1 WD repeat and | 2497 |
| | HMG-box DNA-binding | |
| | protein 1 | |
| Q03933_C369 | HSF2 Heat shock factor | 2498 |
| | protein 2 | |
| Q00535_C290 | CDK5 Cyclin-dependent | 2499 |
| | kinase 5 | |
| B5ME19_C753 | EIF3CL Eukaryotic translation | 2500 |
| | initiation factor 3 subunit | |
| Q9NVP2_C189 | ASF1B Histone chaperone | 2501 |
| | ASF1B | |
| Q13018_C1354 | PLA2R1 Secretory | 2502 |
| | phospholipase A2 receptor | |
| Q15181_C242 | PPA1 Inorganic | 2503 |
| | pyrophosphatase | |
| Q7Z5K2_C1170 | WAPAL Wings apart-like | 2504 |
| | protein homolog | |
| Q13642_C71 | FHL1 Four and a half LIM | 2505 |
| | domains protein 1 | |
| P48556_C274 | PSMD8 26S proteasome non- | 2506 |
| | ATPase regulatory subunit 8 | |
| Q9UBL3_C362 | ASH2L Set1/Ash2 histone | 2507 |
| | methyltransferase complex | |
| | subunit | |
| Q8TBB5_C373 | KLHDC4 Kelch domain- | 2508 |
| | containing protein 4 | |
| P07339_C329 | CTSD Cathepsin D | 2509 |
| Q7Z6Z7_C1401 | HUWE1 E3 ubiquitin-protein | 2510 |
| | ligase HUWE1 | |
| Q16514_C100 | TAF12 Transcription initiation | 2511 |
| | factor TFIID subunit 12 | |
| P63151_C239 | PPP2R2A Serine/threonine- | 2512 |
| | protein phosphatase 2A 55 kDa | |
| | regulatory subunit B alpha | |
| | isoform | |
| Q7L2H7_C175 | EIF3M Eukaryotic translation | 2513 |
| | initiation factor 3 subunit | |
| Q8NHV4_C66 | NEDD1 Protein NEDD1 | 2514 |
| P13010_C339 | XRCC5 X-ray repair cross- | 2515 |
| | complementing protein 5 | |
| P51858_C108 | HDGF Hepatoma-derived | 2516 |
| | growth factor | |
| P26038_C284 | MSN Moesin | 2517 |
| P68402_C206 | PAFAH1B2 Platelet-activating | 2518 |
| | factor acetylhydrolase IB | |
| | subunit | |
| P15407_C97 | FOSL1 Fos-related antigen 1 | 2519 |
| Q12768_C21 | KIAA0196 WASH complex | 2520 |
| | subunit strumpellin | |
| P29144_C28 | TPP2 Tripeptidyl-peptidase 2 | 2521 |
| P78371_C412 | CCT2 T-complex protein 1 | 2522 |
| | subunit beta | |
| O60711_C340 | LPXN Leupaxin | 2523 |
| Q9H9Q2_C110 | COPS7B COP9 signalosome | 2524 |
| | complex subunit 7b | |
| P40261_C165 | NNMT Nicotinamide N- | 2525 |
| | methyltransferase | |
| Q9BWJ5_C76 | SF3B5 Splicing factor 3B | 2526 |
| | subunit 5 | |
| P50479_C44 | PDLIM4 PDZ and LIM | 2527 |
| | domain protein 4 | |
| Q5JRX3_C556 | PITRM1 Presequence | 2528 |
| | protease, mitochondrial | |
| Q9ULE6_C845 | PALD1 Paladin | 2529 |
| Q9NXV6_C178 | CDKN2AIP CDKN2A- | 2530 |
| | interacting protein | |
| P13639_C67 | EEF2 Elongation factor 2 | 2531 |
| P04183_C79 | TK1 Thymidine kinase, | 2532 |
| | cytosolic | |
| Q9H8V3_C221 | ECT2 Protein ECT2 | 2533 |
| Q9NPJ6_C162 | MED4 Mediator of RNA | 2534 |
| | polymerase II transcription | |
| | subunit | |
| Q5VTR2_C924 | RNF20 E3 ubiquitin-protein | 2535 |
| | ligase BRE1A | |
| O00231_C202 | PSMD11 26S proteasome non- | 2536 |
| | ATPase regulatory subunit 11 | |
| P50995_C384 | ANXA11 Annexin A11 | 2537 |
| P62851_C59 | RPS25 40S ribosomal protein | 2538 |
| | S25 | |
| Q12906_C203 | ILF3 Interleukin enhancer- | 2539 |
| | binding factor 3 | |
| Q13315_C2770 | ATM Serine-protein kinase | 2540 |
| | ATM | |
| P22681_C508 | CBL E3 ubiquitin-protein | 2541 |
| | ligase CBL | |
| P35998_C270 | PSMC2 26S protease | 2542 |
| | regulatory subunit 7 | |
| O75570_C137 | MTRF1 Peptide chain release | 2543 |
| | factor 1, mitochondrial | |
| P35579_C1437 | MYH9 Myosin-9 | 2544 |
| P48643_C493 | CCT5 T-complex protein 1 | 2545 |
| | subunit epsilon | |
| P52209_C422 | PGD 6-phosphogluconate | 2546 |
| | dehydrogenase, | |
| | decarboxylating | |
| Q13155_C306 | AIMP2 Aminoacyl tRNA | 2547 |
| | synthase complex-interacting | |
| | multifunctional protein 2 | |
| Q14197_C82 | ICT1 Peptidyl-tRNA hydrolase | 2548 |
| | ICT1, mitochondrial | |
| C9J4G0_C66 | C17orf49 HCG32827, isoform | 2549 |
| | CRA_d | |
| P00338_C185 | LDHA L-lactate | 2550 |
| | dehydrogenase A chain | |
| Q9UBQ0_C41 | VPS29 Vacuolar protein | 2551 |
| | sorting-associated protein 29 | |
| Q15645_C14 | TRIP13 Pachytene checkpoint | 2552 |
| | protein 2 homolog | |
| Q9H1K1_C69 | ISCU Iron-sulfur cluster | 2553 |
| | assembly enzyme ISCU, | |
| | mitochondrial | |
| P30101_C244 | PDIA3 Protein disulfide- | 2554 |
| | isomerase A3 | |
| P09884_C1458 | POLA1 DNA polymerase | 2555 |
| | alpha catalytic subunit | |
| Q9NT62_C182 | ATG3 Ubiquitin-like- | 2556 |
| | conjugating enzyme ATG3 | |
| Q7L591_C323 | DOK3 Docking protein 3 | 2557 |
| Q6IA86_C487 | ELP2 Elongator complex | 2558 |
| | protein 2 | |
| P54819_C40 | AK2 Adenylate kinase 2, | 2559 |
| | mitochondrial | |
| P23610_C110 | F8A3 Factor VIII intron 22 | 2560 |
| | protein | |
| P13861_C101 | PRKAR2A cAMP-dependent | 2561 |
| | protein kinase type II-alpha | |
| | regulatory subunit | |
| Q96ER3_C366 | SAAL1 Protein SAAL1 | 2562 |
| Q92597_C272 | NDRG1 Protein NDRG1 | 2563 |
| Q6P9B6_C13 | KIAA1609 TLD domain- | 2564 |
| | containing protein KIAA1609 | |
| O75663_C14 | TIPRL TIP41-like protein | 2565 |
| Q05086_C49 | UBE3A Ubiquitin-protein | 2566 |
| | ligase E3A | |
| Q14152_C78 | EIF3A Eukaryotic translation | 2567 |
| | initiation factor 3 subunit | |
| P63244_C207 | GNB2L1 Guanine nucleotide- | 2568 |
| | binding protein subunit beta-2- | |
| | like 1 | |
| Q13509_C201 | TUBB3 Tubulin beta-3 chain | 2569 |
| Q9Y6A5_C502 | TACC3 Transforming acidic | 2570 |
| | coiled-coil-containing protein | |
| Q86W42_C314 | THOC6 THO complex subunit | 2571 |
| | 6 homolog | |
| Q92878_C157 | RAD50 DNA repair protein | 2572 |
| | RAD50 | |
| O75879_C228 | PET112 Glutamyl-tRNA(Gln) | 2573 |
| | amidotransferase subunit B, | |
| | mitochondrial | |
| Q9UHB9_C562 | SRP68 Signal recognition | 2574 |
| | particle 68 kDa protein | |
| P41250_C155 | GARS Glycine--tRNA ligase | 2575 |
| P46821_C1814 | MAP1B Microtubule- | 2576 |
| | associated protein 1B | |
| O14980_C723 | XPO1 Exportin-1 | 2577 |
| P13796_C164 | LCP1 Plastin-2 | 2578 |
| P13796_C336 | LCP1 Plastin-2 | 2579 |
| Q9Y2L1_C533 | DIS3 Exosome complex | 2580 |
| | exonuclease RRP44 | |
| Q9NQW6_C96 | ANLN Actin-binding protein | 2581 |
| | anillin | |
| P43246_C176 | MSH2 DNA mismatch repair | 2582 |
| | protein Msh2 | |
| Q9Y4L1_C352 | HYOU1 Hypoxia up-regulated | 2583 |
| | protein 1 | |
| P15153_C105 | RAC2 Ras-related C3 | 2584 |
| | botulinum toxin substrate 2 | |
| Q9H0G5_C232 | NSRP1 Nuclear speckle | 2585 |
| | splicing regulatory protein 1 | |
| P48507_C194 | GCLM Glutamate--cysteine | 2586 |
| | ligase regulatory subunit | |
| Q9BRX5_C188 | GINS3 DNA replication | 2587 |
| | complex GINS protein PSF3 | |
| Q9NR09_C543 | BIRC6 Baculoviral IAP | 2588 |
| | repeat-containing protein 6 | |
| Q13813_C1314 | SPTAN1 Spectrin alpha chain, | 2589 |
| | non-erythrocytic 1 | |
| Q10713_C466 | PMPCA Mitochondrial- | 2590 |
| | processing peptidase subunit | |
| | alpha | |
| Q9H7N4_C1098 | SCAF1 Splicing factor, | 2591 |
| | arginine/serine-rich 19 | |
| Q9NXA8_C303 | SIRT5 NAD-dependent protein | 2592 |
| | deacylase sirtuin-5, | |
| | mitochondrial | |
| P83916_C60 | CBX1 Chromobox protein | 2593 |
| | homolog 1 | |
| O00410_C110 | IPO5 Importin-5 | 2594 |
| P57764_C457 | GSDMD Gasdermin-D | 2595 |
| P15170_C327 | GSPT1 Eukaryotic peptide | 2596 |
| | chain release factor GTP- | |
| | binding | |
| Q9BWD1_C360 | ACAT2 Acetyl-CoA | 2597 |
| | acetyltransferase, cytosolic | |
| P04818_C180 | TYMS Thymidylate synthase | 2598 |
| Q9BQ69_C276 | MACROD1 O-acetyl-ADP- | 2599 |
| | ribose deacetylase MACROD1 | |
| Q01970_C834 | PLCB3 1-phosphatidylinositol | 2600 |
| | 4,5-bisphosphate phosphodie | |
| Q9Y6E0_C394 | STK24 Serine/threonine- | 2601 |
| | protein kinase 24 | |
| P53634_C136 | CTSC Dipeptidyl peptidase 1 | 2602 |
| Q9UGP4_C431 | LIMD1 LIM domain- | 2603 |
| | containing protein 1 | |
| O75369_C455 | FLNB Filamin-B | 2604 |
| P21980_C554 | TGM2 Protein-glutamine | 2605 |
| | gamma-glutamyltransferase 2 | |
| P30281_C47 | CCND3 G1/S-specific cyclin- | 2606 |
| | D3 | |
| P49790_C1129 | NUP153 Nuclear pore | 2607 |
| | complex protein Nup153 | |
| Q93008_C673 | USP9X Probable ubiquitin | 2608 |
| | carboxyl-terminal hydrolase | |
| | FAF | |
| Q9Y613_C31 | FHOD1 FH1/FH2 domain- | 2609 |
| | containing protein 1 | |
| Q9BXB5_C203 | OSBPL10 Oxysterol-binding | 2610 |
| | protein-related protein 10 | |
| P62633_C97 | CNBP Cellular nucleic acid- | 2611 |
| | binding protein | |
| Q14005_C1016 | IL16 Pro-interleukin-16 | 2612 |
| Q9UPQ0_C863 | LIMCH1 LIM and calponin | 2613 |
| | homology domains-containing | |
| | protein | |
| Q9HD26_C408 | GOPC Golgi-associated PDZ | 2614 |
| | and coiled-coil motif- | |
| | containing | |
| P07900_C481 | HSP90AA1 Heat shock protein | 2615 |
| | HSP 90-alpha | |
| P21333_C717 | FLNA Filamin-A | 2616 |
| P21333_C2582 | FLNA Filamin-A | 2617 |
| P13010_C235 | XRCC5 X-ray repair cross- | 2618 |
| | complementing protein 5 | |
| O95347_C46 | SMC2 Structural maintenance | 2619 |
| | of chromosomes protein 2 | |
| P68402_C188 | PAFAH1B2 Platelet-activating | 2620 |
| | factor acetylhydrolase IB | |
| | subunit | |
| P61962_C256 | DCAF7 DDB1- and CUL4- | 2621 |
| | associated factor 7 | |
| P49327_C1118 | FASN Fatty acid synthase | 2622 |
| P49321_C84 | NASP Nuclear autoantigenic | 2623 |
| | sperm protein | |
| Q14019_C52 | COTL1 Coactosin-like protein | 2624 |
| Q9NYK5_C335 | MRPL39 39S ribosomal | 2625 |
| | protein L39, mitochondrial | |
| P55160_C338 | NCKAP1L Nck-associated | 2626 |
| | protein 1-like | |
| P10599_C69 | TXN Thioredoxin | 2627 |
| P14921_C31 | ETS1 Protein C-ets-1 | 2628 |
| Q8NBJ5_C412 | GLT25D1 Procollagen | 2629 |
| | galactosyltransferase 1 | |
| Q13200_C448 | PSMD2 26S proteasome non- | 2630 |
| | ATPase regulatory subunit 2 | |
| Q9ULW0_C364 | TPX2 Targeting protein for | 2631 |
| | Xklp2 | |
| P09914_C138 | IFIT1 Interferon-induced | 2632 |
| | protein with tetratricopeptide | |
| O14744_C449 | PRMT5 Protein arginine N- | 2633 |
| | methyltransferase 5 | |
| Q6NXT1_C230 | ANKRD54 Ankyrin repeat | 2634 |
| | domain-containing protein 54 | |
| Q5VT52_C649 | RPRD2 Regulation of nuclear | 2635 |
| | pre-mRNA domain-containing | |
| | P | |
| P22314_C200 | UBA1 Ubiquitin-like modifier- | 2636 |
| | activating enzyme 1 | |
| P22314_C431 | UBA1 Ubiquitin-like modifier- | 2637 |
| | activating enzyme 1 | |
| Q8IU81_C280 | IRF2BP1 Interferon regulatory | 2638 |
| | factor 2-binding protein 1 | |
| Q5T160_C576 | RARS2 Probable arginine-- | 2639 |
| | tRNA ligase, mitochondrial | |
| Q86UV5_C986 | USP48 Ubiquitin carboxyl- | 2640 |
| | terminal hydrolase 48 | |
| P04083_C343 | ANXA1 Annexin A1 | 2641 |
| Q8NFF5_C236 | FLAD1 FAD synthase | 2642 |
| P04150_C367 | NR3C1 Glucocorticoid | 2643 |
| | receptor | |
| Q9H269_C490 | VPS16 Vacuolar protein | 2644 |
| | sorting-associated protein 16 | |
| | homolog | |
| Q969Z0_C417 | TBRG4 Protein TBRG4 | 2645 |
| Q9ULC4_C113 | MCTS1 Malignant T-cell- | 2646 |
| | amplified sequence 1 | |
| Q15306_C343 | IRF4 Interferon regulatory | 2647 |
| | factor 4 | |
| Q15013_C124 | MAD2L1BP MAD2L1- | 2648 |
| | binding protein | |
| P45974_C532 | USP5 Ubiquitin carboxyl- | 2649 |
| | terminal hydrolase 5 | |
| O95817_C179 | BAG3 BAG family molecular | 2650 |
| | chaperone regulator 3 | |
| Q99797_C687 | MIPEP Mitochondrial | 2651 |
| | intermediate peptidase | |
| P35579_C896 | MYH9 Myosin-9 | 2652 |
| O94855_C853 | SEC24D Protein transport | 2653 |
| | protein Sec24D | |
| O14965_C393 | AURKA Aurora kinase A | 2654 |
| Q9BQA1_C65 | WDR77 Methylosome protein | 2655 |
| | 50 | |
| Q27J81_C284 | INF2 Inverted formin-2 | 2656 |
| P30048_C108 | PRDX3 Thioredoxin- | 2657 |
| | dependent peroxide reductase, | |
| | mitochondrial | |
| P22102_C646 | GART Trifunctional purine | 2658 |
| | biosynthetic protein adenosin | |
| P62316_C63 | SNRPD2 Small nuclear | 2659 |
| | ribonucleoprotein Sm D2 | |
| P09884_C141 | POLA1 DNA polymerase | 2660 |
| | alpha catalytic subunit | |
| Q7L591_C493 | DOK3 Docking protein 3 | 2661 |
| O00159_C161 | MYO1C Unconventional | 2662 |
| | myosin-Ic | |
| O00151_C73 | PDLIM1 PDZ and LIM | 2663 |
| | domain protein 1 | |
| O00151_C263 | PDLIM1 PDZ and LIM | 2664 |
| | domain protein 1 | |
| P62136_C171 | PPP1CA Serine/threonine- | 2665 |
| | protein phosphatase PP1-alpha | |
| | catalytic subunit | |
| P55060_C853 | CSE1L Exportin-2 | 2666 |
| O95232_C58 | LUC7L3 Luc7-like protein 3 | 2667 |
| P29590_C338 | PML Protein PML | 2668 |
| Q8IXK0_C673 | PHC2 Polyhomeotic-like | 2669 |
| | protein 2 | |
| Q14980_C1729 | NUMA1 Nuclear mitotic | 2670 |
| | apparatus protein 1 | |
| P63244_C286 | GNB2L1 Guanine nucleotide- | 2671 |
| | binding protein subunit beta-2- | |
| | like 1 | |
| P62333_C347 | PSMC6 26S protease | 2672 |
| | regulatory subunit 10B R | |
| Q13501_C113 | SQSTM1 Sequestosome-1 | 2673 |
| P60866_C36 | RPS20 40S ribosomal protein | 2674 |
| | S20 | |
| Q969E8_C17 | TSR2 Pre-rRNA-processing | 2675 |
| | protein TSR2 homolog | |
| Q93052_C262 | LPP Lipoma-preferred partner | 2676 |
| P08758_C316 | ANXA5 Annexin A5 | 2677 |
| O75616_C150 | ERAL1 GTPase Era, | 2678 |
| | mitochondrial | |
| Q9UHB9_C525 | SRP68 Signal recognition | 2679 |
| | particle 68 kDa protein | |
| Q9H223_C175 | EHD4 EH domain-containing | 2680 |
| | protein 4 | |
| P08107_C603 | HSPA1B Heat shock 70 kDa | 2681 |
| | protein 1A/1B | |
| P27708_C183 | CAD CAD protein | 2682 |
| Q9Y4L1_C805 | HYOU1 Hypoxia up-regulated | 2683 |
| | protein 1 | |
| P26358_C1125 | DNMT1 DNA (cytosine-5)- | 2684 |
| | methyltransferase 1 | |
| P36954_C52 | POLR2I DNA-directed RNA | 2685 |
| | polymerase II subunit RPB9 | |
| P49748_C156 | ACADVL Very long-chain | 2686 |
| | specific acyl-CoA | |
| | dehydrogenase, mitochondrial | |
| Q9H2G2_C1212 | SLK STE20-like | 2687 |
| | serine/threonine-protein kinase | |
| P35268_C25 | RPL22 60S ribosomal protein | 2688 |
| | L22 | |
| P46939_C539 | UTRN Utrophin | 2689 |
| P49368_C173 | CCT3 T-complex protein 1 | 2690 |
| | subunit gamma | |
| P50135_C82 | HNMT Histamine N- | 2691 |
| | methyltransferase | |
| Q6Y7W6_C938 | GIGYF2 PERQ amino acid- | 2692 |
| | rich with GYF domain- | |
| | containing protein 2 | |
| O95861_C59 | BPNT1 3(2),5-bisphosphate | 2693 |
| | nucleotidase 1 | |
| P49023_C290 | PXN Paxillin | 2694 |
| Q99873_C216 | PRMT1 Protein arginine N- | 2695 |
| | methyltransferase 1 | |
| P02765_C132 | AHSG Alpha-2-HS- | 2696 |
| | glycoprotein | |
| Q9H3M7_C120 | TXNIP Thioredoxin- | 2697 |
| | interacting protein | |
| Q9UPN3_C4825 | MACF1 Microtubule-actin | 2698 |
| | cross-linking factor 1, isoforms | |
| Q9UPN9_C150 | TRIM33 E3 ubiquitin-protein | 2699 |
| | ligase TRIM33 | |
| O75369_C1158 | FLNB Filamin-B | 2700 |
| P56545_C140 | CTBP2 C-terminal-binding | 2701 |
| | protein 2 | |
| Q00341_C948 | HDLBP Vigilin | 2702 |
| P35250_C171 | RFC2 Replication factor C | 2703 |
| | subunit 2 | |
| P46013_C1285 | MKI67 Antigen KI-67 | 2704 |
| O75083_C225 | WDR1 WD repeat-containing | 2705 |
| | protein 1 | |
| O43252_C165 | PAPSS1 Bifunctional 3- | 2706 |
| | phosphoadenosine 5- | |
| | phosphosulfate | |
| Q16512_C317 | PKN1 Serine/threonine-protein | 2707 |
| | kinase N1 | |
| Q16881_C339 | TXNRD1 Thioredoxin | 2708 |
| | reductase 1, cytoplasmic | |
| P21333_C478 | FLNA Filamin-A | 2709 |
| P21333_C2378 | FLNA Filamin-A | 2710 |
| O00268_C867 | TAF4 Transcription initiation | 2711 |
| | factor TFIID subunit 4 | |
| O95340_C117 | PAPSS2 Bifunctional 3- | 2712 |
| | phosphoadenosine 5- | |
| | phosphosulfate | |
| P42765_C382 | ACAA2 3-ketoacyl-CoA | 2713 |
| | thiolase, mitochondrial | |
| Q9UI12_C85 | ATP6V1H V-type proton | 2714 |
| | ATPase subunit H | |
| Q05655_C127 | PRKCD Protein kinase C delta | 2715 |
| | type | |
| P42166_C629 | TMPO Lamina-associated | 2716 |
| | polypeptide 2, isoform alpha | |
| O14929_C27 | HAT1 Histone | 2717 |
| | acetyltransferase type B | |
| | catalytic subunit L | |
| Q9NYK5_C133 | MRPL39 39S ribosomal | 2718 |
| | protein L39, mitochondrial | |
| O60234_C96 | GMFG Glia maturation factor | 2719 |
| | gamma | |
| F8VZW7_C74 | Uncharacterized protein | 2720 |
| Q12888_C1040 | TP53BP1 Tumor suppressor | 2721 |
| | p53-binding protein 1 | |
| Q5JTZ9_C609 | AARS2 Alanine--tRNA ligase, | 2722 |
| | mitochondrial | |
| Q9Y383_C44 | LUC7L2 Putative RNA- | 2723 |
| | binding protein Luc7-like 2 | |
| Q6P1R3_C99 | MSANTD2 Myb/SANT-like | 2724 |
| | DNA-binding domain- | |
| | containing protei | |
| Q99832_C364 | CCT7 T-complex protein 1 | 2725 |
| | subunit eta | |
| Q9NRW4_C124 | DUSP22 Dual specificity | 2726 |
| | protein phosphatase 22 | |
| Q8N163_C238 | KIAA1967 DBIRD complex | 2727 |
| | subunit KIAA1967 | |
| Q8N163_C443 | KIAA1967 DBIRD complex | 2728 |
| | subunit KIAA1967 | |
| Q15149_C1136 | PLEC Plectin | 2729 |
| Q15149_C4454 | PLEC Plectin | 2730 |
| P31146_C285 | CORO1A Coronin-1A | 2731 |
| Q16555_C248 | DPYSL2 | 2732 |
| | Dihydropyrimidinase-related | |
| | protein 2 | |
| Q99567_C713 | NUP88 Nuclear pore complex | 2733 |
| | protein Nup88 | |
| P21964_C119 | COMT Catechol O- | 2734 |
| | methyltransferase | |
| P62857_C27 | RPS28 40S ribosomal protein | 2735 |
| | S28 | |
| Q9P287_C275 | BCCIP BRCA2 and | 2736 |
| | CDKN1A-interacting protein | |
| Q7LBC6_C1470 | KDM3B Lysine-specific | 2737 |
| | demethylase 3B | |
| P62140_C171 | PPP1CB Serine/threonine- | 2738 |
| | protein phosphatase PP1-beta | |
| | catalytic subunit | |
| P33764_C68 | S100A3 Protein S100-A3 | 2739 |
| Q9UBQ7_C123 | GRHPR Glyoxylate | 2740 |
| | reductase/hydroxypyruvate | |
| | reductase | |
| Q9UJY4_C343 | GGA2 ADP-ribosylation | 2741 |
| | factor-binding protein GGA2 | |
| O60841_C1092 | EIF5B Eukaryotic translation | 2742 |
| | initiation factor 5B | |
| Q92945_C296 | KHSRP Far upstream element- | 2743 |
| | binding protein 2 | |
| Q9HAV4_C941 | XPO5 Exportin-5 | 2744 |
| O14964_C185 | HGS Hepatocyte growth | 2745 |
| | factor-regulated tyrosine | |
| | kinase | |
| P23526_C195 | AHCY | 2746 |
| | Adenosylhomocysteinase | |
| P52209_C289 | PGD 6-phosphogluconate | 2747 |
| | dehydrogenase, | |
| | decarboxylating | |
| Q9Y697_C381 | NFS1 Cysteine desulfurase, | 2748 |
| | mitochondrial | |
| Q9Y520_C177 | PRRC2C Protein PRRC2C | 2749 |
| Q9H910_C118 | HN1L Hematological and | 2750 |
| | neurological expressed 1-like | |
| | protein | |
| Q6FI81_C92 | CIAPIN1 Anamorsin | 2751 |
| Q8WZA9_C340 | IRGQ Immunity-related | 2752 |
| | GTPase family Q protein | |
| Q8NDH3_C81 | NPEPL1 Probable | 2753 |
| | aminopeptidase NPEPL1 | |
| O00571_C317 | DDX3X ATP-dependent RNA | 2754 |
| | helicase DDX3X | |
| P05091_C179 | ALDH2 Aldehyde | 2755 |
| | dehydrogenase, mitochondrial | |
| P05091_C472 | ALDH2 Aldehyde | 2756 |
| | dehydrogenase, mitochondrial | |
| O15235_C132 | MRPS12 28S ribosomal | 2757 |
| | protein S12, mitochondrial | |
| P42704_C130 | LRPPRC Leucine-rich PPR | 2758 |
| | motif-containing protein, | |
| | mitochondrial | |
| Q99590_C566 | SCAF11 Protein SCAF11 | 2759 |
| Q9NR56_C19 | MBNL1 Muscleblind-like | 2760 |
| | protein 1 | |
| Q9P2E9_C1038 | RRBP1 Ribosome-binding | 2761 |
| | protein 1 | |
| P52888_C682 | THOP1 Thimet oligopeptidase | 2762 |
| P55212_C264 | CASP6 Caspase-6 | 2763 |
| Q6IBW4_C342 | NCAPH2 Condensin-2 | 2764 |
| | complex subunit H2 | |
| P18754_C352 | RCC1 Regulator of | 2765 |
| | chromosome condensation | |
| Q8TB52_C433 | FBXO30 F-box only protein | 2766 |
| | 30 | |
| Q32MZ4_C644 | LRRFIP1 Leucine-rich repeat | 2767 |
| | flightless-interacting protein | |
| Q9NYL2_C150 | MLTK Mitogen-activated | 2768 |
| | protein kinase kinase kinase | |
| | MLT | |
| Q15393_C1156 | SF3B3 Splicing factor 3B | 2769 |
| | subunit 3 | |
| P63244_C168 | GNB2L1 Guanine nucleotide- | 2770 |
| | binding protein subunit beta-2- | |
| | like 1 | |
| Q53EL6_C275 | PDCD4 Programmed cell | 2771 |
| | death protein 4 | |
| P56537_C152 | EIF6 Eukaryotic translation | 2772 |
| | initiation factor 6 | |
| P09622_C484 | DLD Dihydrolipoyl | 2773 |
| | dehydrogenase, mitochondrial | |
| Q9Y314_C185 | NOSIP Nitric oxide synthase- | 2774 |
| | interacting protein | |
| Q96P16_C151 | RPRD1A Regulation of | 2775 |
| | nuclear pre-mRNA domain- | |
| | containing p | |
| P46821_C2041 | MAP1B Microtubule- | 2776 |
| | associated protein 1B | |
| O60343_C266 | TBC1D4 TBC1 domain family | 2777 |
| | member 4 | |
| Q96EB1_C361 | ELP4 Elongator complex | 2778 |
| | protein 4 | |
| Q86WB0_C102 | ZC3HC1 Nuclear-interacting | 2779 |
| | partner of ALK | |
| P49915_C104 | GMPS GMP synthase | 2780 |
| O75608_C144 | LYPLA1 Acyl-protein | 2781 |
| | thioesterase 1 | |
| P11498_C131 | PC Pyruvate carboxylase, | 2782 |
| | mitochondrial | |
| P11498_C752 | PC Pyruvate carboxylase, | 2783 |
| | mitochondrial | |
| Q53H96_C266 | PYCRL Pyrroline-5- | 2784 |
| | carboxylate reductase 3 | |
| Q96DE5_C55 | ANAPC16 Anaphase- | 2785 |
| | promoting complex subunit 16 | |
| Q96KB5_C112 | PBK Lymphokine-activated | 2786 |
| | killer T-cell-originated prot | |
| P55084_C458 | HADHB Trifunctional enzyme | 2787 |
| | subunit beta, mitochondrial | |
| P55735_C234 | SEC13 Protein SEC13 | 2788 |
| | homolog | |
| P56192_C287 | MARS Methionine--tRNA | 2789 |
| | ligase, cytoplasmic | |
| P07384_C115 | CAPN1 Calpain-1 catalytic | 2790 |
| | subunit | |
| P08559_C222 | PDHA1 Pyruvate | 2791 |
| | dehydrogenase E1 component | |
| | subunit alpha, | |
| Q99714_C91 | HSD17B10 3-hydroxyacyl- | 2792 |
| | CoA dehydrogenase type-2 | |
| P33992_C197 | MCM5 DNA replication | 2793 |
| | licensing factor MCM5 | |
| P49757_C611 | NUMB Protein numb homolog | 2794 |
| O15269_C438 | SPTLC1 Serine | 2795 |
| | palmitoyltransferase 1 | |
| O75935_C173 | DCTN3 Dynactin subunit 3 | 2796 |
| Q8IU85_C182 | CAMK1D | 2797 |
| | Calcium/calmodulin-dependent | |
| | protein kinase type 1 | |
| Q05519_C455 | SRSF11 Serine/arginine-rich | 2798 |
| | splicing factor 11 | |
| P36578_C125 | RPL4 60S ribosomal protein | 2799 |
| | L4 | |
| Q96E14_C95 | RMI2 RecQ-mediated genome | 2800 |
| | instability protein 2 | |
| Q96N67_C457 | DOCK7 Dedicator of | 2801 |
| | cytokinesis protein 7 | |
| Q7Z5K2_C160 | WAPAL Wings apart-like | 2802 |
| | protein homolog | |
| P41214_C489 | EIF2D Eukaryotic translation | 2803 |
| | initiation factor 2D | |
| O43242_C483 | PSMD3 26S proteasome non- | 2804 |
| | ATPase regulatory subunit 3 | |
| O75369_C1280 | FLNB Filamin-B | 2805 |
| Q8IWI9_C2974 | MGA MAX gene-associated | 2806 |
| | protein | |
| O15067_C785 | PFAS | 2807 |
| | Phosphoribosylformylglycinamidine | |
| | synthase | |
| Q96GX9_C16 | APIP Probable | 2808 |
| | methylthioribulose-1- | |
| | phosphate dehydratase | |
| Q9BXP5_C412 | SRRT Serrate RNA effector | 2809 |
| | molecule homolog | |
| O75083_C382 | WDR1 WD repeat-containing | 2810 |
| | protein 1 | |
| Q8WVV9_C535 | HNRPLL Heterogeneous | 2811 |
| | nuclear ribonucleoprotein L- | |
| | like | |
| Q7Z6Z7_C1891 | HUWE1 E3 ubiquitin-protein | 2812 |
| | ligase HUWE1 | |
| Q7Z6Z7_C3296 | HUWE1 E3 ubiquitin-protein | 2813 |
| | ligase HUWE1 | |
| Q14008_C1500 | CKAP5 Cytoskeleton- | 2814 |
| | associated protein 5 | |
| Q9H3P2_C44 | WHSC2 Negative elongation | 2815 |
| | factor A | |
| Q8NB37_C154 | PDDC1 Parkinson disease 7 | 2816 |
| | domain-containing protein 1 | |
| P48047_C141 | ATP5O ATP synthase subunit | 2817 |
| | O, mitochondrial | |
| Q8IUI8_C95 | CRLF3 Cytokine receptor-like | 2818 |
| | factor 3 | |
| Q9H6S3_C261 | EPS8L2 Epidermal growth | 2819 |
| | factor receptor kinase substrate | |
| P21333_C53 | FLNA Filamin-A | 2820 |
| Q5T4S7_C3864 | UBR4 E3 ubiquitin-protein | 2821 |
| | ligase UBR4 | |
| P46060_C82 | RANGAP1 Ran GTPase- | 2822 |
| | activating protein 1 | |
| O14929_C299 | HAT1 Histone | 2823 |
| | acetyltransferase type B | |
| | catalytic subunit | |
| P48059_C272 | LIMS1 LIM and senescent cell | 2824 |
| | antigen-like-containing | |
| | domains 1 | |
| O15042_C65 | U2SURP U2 snRNP- | 2825 |
| | associated SURP motif- | |
| | containing protein | |
| Q7L2J0_C244 | MEPCE 7SK snRNA | 2826 |
| | methylphosphate capping | |
| | enzyme | |
| Q4G176_C399 | ACSF3 Acyl-CoA synthetase | 2827 |
| | family member 3, | |
| | mitochondrial | |
| Q9H1B7_C504 | IRF2BPL Interferon regulatory | 2828 |
| | factor 2-binding protein-lik | |
| O95425_C26 | SVIL Supervillin | 2829 |
| O75351_C240 | VPS4B Vacuolar protein | 2830 |
| | sorting-associated protein 4B | |
| Q96DC7_C57 | TMCO6 Transmembrane and | 2831 |
| | coiled-coil domain-containing | |
| | pr | |
| Q9ULW0_C594 | TPX2 Targeting protein for | 2832 |
| | Xklp2 | |
| P50502_C209 | ST13 Hsc70-interacting | 2833 |
| | protein | |
| P31146_C51 | CORO1A Coronin-1A | 2834 |
| P31146_C345 | CORO1A Coronin-1A | 2835 |
| Q9H2M9_C67 | RAB3GAP2 Rab3 GTPase- | 2836 |
| | activating protein non-catalytic | |
| | subunit | |
| Q9UKV3_C546 | ACIN1 Apoptotic chromatin | 2837 |
| | condensation inducer in the | |
| | nucleus | |
| P78527_C1229 | PRKDC DNA-dependent | 2838 |
| | protein kinase catalytic subunit | |
| P78527_C1364 | PRKDC DNA-dependent | 2839 |
| | protein kinase catalytic subunit | |
| P42566_C657 | EPS15 Epidermal growth | 2840 |
| | factor receptor substrate 15 | |
| P61981_C112 | YWHAG 14-3-3 protein | 2841 |
| | gamma | |
| P20810_C381 | CAST Calpastatin | 2842 |
| P55036_C58 | PSMD4 26S proteasome non- | 2843 |
| | ATPase regulatory subunit 4 | |
| P50991_C295 | CCT4 T-complex protein 1 | 2844 |
| | subunit delta | |
| Q96TA1_C230 | FAM129B Niban-like protein | 2845 |
| | 1 | |
| P49848_C141 | TAF6 Transcription initiation | 2846 |
| | factor TFIID subunit 6 | |
| Q14315_C1066 | FLNC Filamin-C | 2847 |
| O94925_C525 | GLS Glutaminase kidney | 2848 |
| | isoform, mitochondrial | |
| Q92796_C519 | DLG3 Disks large homolog 3 | 2849 |
| O95571_C219 | ETHE1 Protein ETHE1, | 2850 |
| | mitochondrial | |
| Q9ULU8_C800 | CADPS Calcium-dependent | 2851 |
| | secretion activator 1 | |
| P50453_C335 | SERPINB9 Serpin B9 | 2852 |
| O95999_C215 | BCL10 B-cell | 2853 |
| | lymphoma/leukemia 10 | |
| Q9H4Z3_C29 | PCIF1 Phosphorylated CTD- | 2854 |
| | interacting factor 1 | |
| P22692_C204 | IGFBP4 Insulin-like growth | 2855 |
| | factor-binding protein 4 | |
| O00148_C223 | DDX39A ATP-dependent | 2856 |
| | RNA helicase DDX39A | |
| Q5VYK3_C814 | ECM29 Proteasome-associated | 2857 |
| | protein ECM29 homolog | |
| Q9NVE7_C537 | PANK4 Pantothenate kinase 4 | 2858 |
| O95801_C63 | TTC4 Tetratricopeptide repeat | 2859 |
| | protein 4 | |
| Q14204_C2142 | DYNC1H1 Cytoplasmic | 2860 |
| | dynein 1 heavy chain 1 | |
| P48735_C418 | IDH2 Isocitrate dehydrogenase | 2861 |
| O15371_C258 | EIF3D Eukaryotic translation | 2862 |
| | initiation factor 3 subunit | |
| O00571_C128 | DDX3X ATP-dependent RNA | 2863 |
| | helicase DDX3X | |
| Q8WVC0_C530 | LEO1 RNA polymerase- | 2864 |
| | associated protein LEO1 | |
| O15235_C64 | MRPS12 28S ribosomal | 2865 |
| | protein S12, mitochondrial | |
| Q9P2I0_C621 | CPSF2 Cleavage and | 2866 |
| | polyadenylation specificity | |
| | factor subunit | |
| Q9H4X1_C33 | RGCC Regulator of cell cycle | 2867 |
| | RGCC | |
| Q7L5N1_C299 | COPS6 COP9 signalosome | 2868 |
| | complex subunit 6 | |
| Q6NYC8_C611 | PPP1R18 Phostensin | 2869 |
| P22626_C50 | HNRNPA2B1 Heterogeneous | 2870 |
| | nuclear ribonucleoproteins | |
| | A2/B1 | |
| P55072_C105 | VCP Transitional endoplasmic | 2871 |
| | reticulum ATPase | |
| P11912_C196 | CD79A B-cell antigen receptor | 2872 |
| | complex-associated protein | |
| Q9NSE4_C91 | IARS2 Isoleucine--tRNA | 2873 |
| | ligase, mitochondrial | |
| Q9NSE4_C819 | IARS2 Isoleucine--tRNA | 2874 |
| | ligase, mitochondrial | |
| Q9NYF8_C688 | BCLAF1 Bcl-2-associated | 2875 |
| | transcription factor 1 | |
| Q15393_C750 | SF3B3 Splicing factor 3B | 2876 |
| | subunit 3 | |
| Q16576_C166 | RBBP7 Histone-binding | 2877 |
| | protein RBBP7 | |
| Q9NZB2_C279 | FAM120A Constitutive | 2878 |
| | coactivator of PPAR-gamma- | |
| | like protein 1 | |
| Q15052_C25 | ARHGEF6 Rho guanine | 2879 |
| | nucleotide exchange factor 6 | |
| P09622_C80 | DLD Dihydrolipoyl | 2880 |
| | dehydrogenase, mitochondrial | |
| Q96EP0_C551 | RNF31 E3 ubiquitin-protein | 2881 |
| | ligase RNF31 | |
| P50579_C121 | METAP2 Methionine | 2882 |
| | aminopeptidase 2 | |
| Q7Z434_C133 | MAVS Mitochondrial | 2883 |
| | antiviral-signaling protein | |
| Q29RF7_C327 | PDS5A Sister chromatid | 2884 |
| | cohesion protein PDS5 | |
| | homolog A | |
| Q86WB0_C406 | ZC3HC1 Nuclear-interacting | 2885 |
| | partner of ALK | |
| Q9Y490_C1953 | TLN1 Talin-1 | 2886 |
| P43243_C230 | MATR3 Matrin-3 | 2887 |
| P62913_C72 | RPL11 60S ribosomal protein | 2888 |
| | L11 | |
| O00422_C26 | SAP18 Histone deacetylase | 2889 |
| | complex subunit SAP18 | |
| O60921_C200 | HUS1 Checkpoint protein | 2890 |
| | HUS1 | |
| O96017_C385 | CHEK2 Serine/threonine- | 2891 |
| | protein kinase Chk2 | |
| P36969_C93 | GPX4 Phospholipid | 2892 |
| | hydroperoxide glutathione | |
| | peroxidase, | |
| Q96IZ0_C173 | PAWR PRKC apoptosis WT1 | 2893 |
| | regulator protein | |
| P36871_C101 | PGM1 Phosphoglucomutase-1 | 2894 |
| Q9BXF6_C337 | RAB11FIP5 Rab11 family- | 2895 |
| | interacting protein 5 | |
| Q99873_C262 | PRMT1 Protein arginine N- | 2896 |
| | methyltransferase 1 | |
| P09382_C43 | LGALS1 Galectin-1 | 2897 |
| O75934_C132 | BCAS2 Pre-mRNA-splicing | 2898 |
| | factor SPF27 | |
| Q92538_C685 | GBF1 Golgi-specific brefeldin | 2899 |
| | A-resistance guanine | |
| | nucleotide exchange factor 1 | |
| P55039_C99 | DRG2 Developmentally- | 2900 |
| | regulated GTP-binding protein | |
| | 2 | |
| O43414_C285 | ERI3 ERI1 exoribonuclease 3 | 2901 |
| Q14974_C228 | KPNB1 Importin subunit beta- | 2902 |
| | 1 | |
| P30050_C17 | RPL12 60S ribosomal protein | 2903 |
| | L12 | |
| P78347_C475 | GTF2I General transcription | 2904 |
| | factor II-I | |
| Q9H3M7_C247 | TXNIP Thioredoxin- | 2905 |
| | interacting protein | |
| P08047_C68 | SP1 Transcription factor Sp1 | 2906 |
| O43242_C210 | PSMD3 26S proteasome non- | 2907 |
| | ATPase regulatory subunit 3 | |
| Q9UPN3_C4206 | MACF1 Microtubule-actin | 2908 |
| | cross-linking factor 1, isoforms | |
| Q14103_C126 | HNRNPD Heterogeneous | 2909 |
| | nuclear ribonucleoprotein D0 | |
| Q9BY32_C33 | ITPA Inosine triphosphate | 2910 |
| | pyrophosphatase | |
| Q9BQ52_C670 | ELAC2 Zinc | 2911 |
| | phosphodiesterase ELAC | |
| | protein 2 | |
| O15067_C318 | PFAS | 2912 |
| | Phosphoribosylformylglycinamidine | |
| | synthase | |
| Q9H9J2_C53 | MRPL44 39S ribosomal | 2913 |
| | protein L44, mitochondrial | |
| Q00610_C151 | CLTC Clathrin heavy chain 1 | 2914 |
| P09497_C199 | CLTB Clathrin light chain B | 2915 |
| Q9Y617_C224 | PSAT1 Phosphoserine | 2916 |
| | aminotransferase | |
| P46013_C1007 | MKI67 Antigen KI-67 | 2917 |
| Q9BSD7_C101 | NTPCR Cancer-related | 2918 |
| | nucleoside-triphosphatase | |
| Q7Z6Z7_C349 | HUWE1 E3 ubiquitin-protein | 2919 |
| | ligase HUWE1 | |
| Q14919_C54 | DRAP1 Dr1-associated | 2920 |
| | corepressor | |
| Q9UNE7_C199 | STUB1 E3 ubiquitin-protein | 2921 |
| | ligase CHIP | |
| Q16881_C515 | TXNRD1 Thioredoxin | 2922 |
| | reductase 1, cytoplasmic | |
| P09936_C220 | UCHL1 Ubiquitin carboxyl- | 2923 |
| | terminal hydrolase isozyme L1 | |
| Q9NZJ9_C147 | NUDT4 Diphosphoinositol | 2924 |
| | polyphosphate | |
| | phosphohydrolase 2 | |
| Q5T4S7_C2554 | UBR4 E3 ubiquitin-protein | 2925 |
| | ligase UBR4 | |
| Q96C19_C53 | EFHD2 EF-hand domain- | 2926 |
| | containing protein D2 | |
| Q8N6M0_C292 | OTUD6B OTU domain- | 2927 |
| | containing protein 6B | |
| Q8N201_C1633 | INTS1 Integrator complex | 2928 |
| | subunit 1 | |
| O95294_C301 | RASAL1 RasGAP-activating- | 2929 |
| | like protein 1 | |
| Q9UKX7_C181 | NUP50 Nuclear pore complex | 2930 |
| | protein Nup50 | |
| Q92616_C1595 | GCN1L1 Translational | 2931 |
| | activator GCN1 | |
| P49327_C2024 | FASN Fatty acid synthase | 2932 |
| Q16877_C106 | PFKFB4 6-phosphofructo-2- | 2933 |
| | kinase/fructose-2,6- | |
| | bisphosphatase | |
| Q96JP5_C182 | ZFP91 E3 ubiquitin-protein | 2934 |
| | lgase ZFP91 | |
| Q99538_C50 | LGMN Legumain | 2935 |
| Q96RS6_C32 | NUDCD1 NudC domain- | 2936 |
| | containing protein 1 | |
| Q96RS6_C513 | NUDCD1 NudC domain- | 2937 |
| | containing protein 1 | |
| Q8TBC4_C28 | UBA3 NEDD8-activating | 2938 |
| | enzyme E1 catalytic subunit | |
| Q14839_C1827 | CHD4 Chromodomain- | 2939 |
| | helicase-DNA-binding protein | |
| | 4 | |
| Q9Y2D5_C205 | AKAP2 A-kinase anchor | 2940 |
| | protein 2 | |
| Q9UQ35_C956 | SRRM2 Serine/arginine | 2941 |
| | repetitive matrix protein 2 | |
| O60711_C358 | LPXN Leupaxin | 2942 |
| Q00013_C454 | MPP1 55 kDa erythrocyte | 2943 |
| | membrane protein | |
| Q6P1L8_C57 | MRPL14 39S ribosomal | 2944 |
| | protein L14, mitochondrial | |
| Q92973_C620 | TNPO1 Transportin-1 | 2945 |
| P42575_C320 | CASP2 Caspase-2 | 2946 |
| P35520_C431 | CBS Cystathionine beta- | 2947 |
| | synthase | |
| O95425_C1237 | SV1L Supervillin | 2948 |
| Q9Y305_C299 | ACOT9 Acyl-coenzyme A | 2949 |
| | thioesterase 9, mitochondrial | |
| P32321_C83 | DCTD Deoxycytidylate | 2950 |
| | deaminase | |
| P61158_C189 | ACTR3 Actin-related protein 3 | 2951 |
| P11413_C158 | G6PD Glucose-6-phosphate 1- | 2952 |
| | dehydrogenase | |
| P04181_C93 | OAT Ornithine | 2953 |
| | aminotransferase, | |
| | mitochondrial | |
| P15880_C182 | RPS2 40S ribosomal protein | 2954 |
| | S2 | |
| P01871_C88 | IGHM Ig mu chain C region | 2955 |
| P50991_C120 | CCT4 T-complex protein 1 | 2956 |
| | subunit delta | |
| P57678_C210 | GEMIN4 Gem-associated | 2957 |
| | protein 4 | |
| O94925_C287 | GLS Glutaminase kidney | 2958 |
| | isoform, mitochondrial | |
| Q8N3D4_C1364 | EHBP1L1 EH domain-binding | 2959 |
| | protein 1-like protein 1 | |
| Q8IUF8_C19 | MINA MYC-induced nuclear | 2960 |
| | antigen | |
| Q9NXH9_C376 | TRMT1 tRNA (guanine(26)- | 2961 |
| | N(2))-dimethyltransferase | |
| P00505_C382 | GOT2 Aspartate | 2962 |
| | aminotransferase, | |
| | mitochondrial | |
| A0JLT2_C62 | MED19 Mediator of RNA | 2963 |
| | polymerase II transcription | |
| | subunit | |
| O15382_C135 | BCAT2 Branched-chain- | 2964 |
| | amino-acid aminotransferase, | |
| | mitochondrial | |
| P52789_C606 | HK2 Hexokinase-2 | 2965 |
| Q9Y3C8_C116 | UFC1 Ubiquitin-fold modifier- | 2966 |
| | conjugating enzyme 1 | |
| Q13439_C1269 | GOLGA4 Golgin subfamily A | 2967 |
| | member 4 | |
| P63279_C43 | UBE2I SUMO-conjugating | 2968 |
| | enzyme UBC9 | |
| P60900_C47 | PSMA6 Proteasome subunit | 2969 |
| | alpha type-6 | |
| Q9H0L4_C222 | CSTF2T Cleavage stimulation | 2970 |
| | factor subunit 2 tau variant | |
| Q86UX7_C128 | FERMT3 Fermitin family | 2971 |
| | homolog 3 | |
| Q15003_C714 | NCAPH Condensin complex | 2972 |
| | subunit 2 | |
| O60684_C253 | KPNA6 Importin subunit | 2973 |
| | alpha-7 | |
| O75643_C1580 | SNRNP200 U5 small nuclear | 2974 |
| | ribonucleoprotein 200 kDa | |
| | helicas | |
| O43765_C129 | SGTA Small glutamine-rich | 2975 |
| | tetratricopeptide repeat- | |
| | containing, alpha | |
| Q9GZV5_C363 | WWTR1 WW domain- | 2976 |
| | containing transcription | |
| | regulator protein 1 | |
| P27816_C654 | MAP4 Microtubule-associated | 2977 |
| | protein 4 | |
| Q14847_C53 | LASP1 LIM and SH3 domain | 2978 |
| | protein 1 | |
| O15371_C195 | EIF3D Eukaryotic translation | 2979 |
| | initiation factor 3 subunit | |
| P10768_C181 | ESD S-formylglutathione | 2980 |
| | hydrolase | |
| P52895_C242 | AKR1C2 Aldo-keto reductase | 2981 |
| | family 1 member C2 | |
| Q9UBE0_C303 | SAE1 SUMO-activating | 2982 |
| | enzyme subunit 1 | |
| P29590_C204 | PML Protein PML | 2983 |
| O15231_C262 | ZNF185 Zinc finger protein | 2984 |
| | 185 | |
| O75251_C183 | NDUFS7 NADH | 2985 |
| | dehydrogenase | |
| Q01082_C1900 | SPTBN1 Spectrin beta chain, | 2986 |
| | non-erythrocytic 1 | |
| Q9UHR5_C127 | SAP30BP SAP30-binding | 2987 |
| | protein | |
| Q9NR56_C43 | MBNL1 Muscleblind-like | 2988 |
| | protein 1 | |
| Q9P2E9_C892 | RRBP1 Ribosome-binding | 2989 |
| | protein 1 | |
| Q9P2E9_C933 | RRBP1 Ribosome-binding | 2990 |
| | protein 1 | |
| Q9P2E9_C1057 | RRBP1 Ribosome-binding | 2991 |
| | protein 1 | |
| P13640_C38 | MT1G Metallothionein-1G | 2992 |
| Q9NSE4_C311 | IARS2 Isoleucine--tRNA | 2993 |
| | ligase, mitochondrial | |
| Q9Y2X7_C576 | GIT1 ARF GTPase-activating | 2994 |
| | protein GIT1 | |
| Q9H840_C44 | GEMIN7 Gem-associated | 2995 |
| | protein 7 | |
| P14678_C45 | SNRPB Small nuclear | 2996 |
| | ribonucleoprotein-associated | |
| | protein | |
| P28340_C428 | POLD1 DNA polymerase delta | 2997 |
| | catalytic subunit | |
| P28347_C53 | TEAD1 Transcriptional | 2998 |
| | enhancer factor TEF-1 | |
| P32780_C246 | GTF2H1 General transcription | 2999 |
| | factor IIH subunit 1 | |
| Q86VP6_C71 | CAND1 Cullin-associated | 3000 |
| | NEDD8-dissociated protein 1 | |
| Q92597_C394 | NDRG1 Protein NDRG1 | 3001 |
| Q8IW35_C484 | CEP97 Centrosomal protein of | 3002 |
| | 97 kDa | |
| Q96JB5_C216 | CDK5RAP3 CDK5 regulatory | 3003 |
| | subunit-associated protein 3 | |
| Q96T51_C320 | RUFY1 RUN and FYVE | 3004 |
| | domain-containing protein 1 | |
| Q13503_C29 | MED21 Mediator of RNA | 3005 |
| | polymerase II transcription | |
| | subunit | |
| P49721_C46 | PSMB2 Proteasome subunit | 3006 |
| | beta type-2 | |
| O75376_C2056 | NCOR1 Nuclear receptor | 3007 |
| | corepressor 1 | |
| Q86UP2_C1105 | KTN1 Kinectin | 3008 |
| Q8TB45_C247 | DEPTOR DEP domain- | 3009 |
| | containing mTOR-interacting | |
| | protein | |
| P41252_C87 | IARS Isoleucine--tRNA ligase, | 3010 |
| | cytoplasmic | |
| P41252_C120 | IARS Isoleucine--tRNA ligase, | 3011 |
| | cytoplasmic | |
| P41252_C433 | IARS Isoleucine--tRNA ligase, | 3012 |
| | cytoplasmic | |
| Q06203_C496 | PPAT | 3013 |
| | Amidophosphoribosyltransferase | |
| Q15437_C767 | SEC23B Protein transport | 3014 |
| | protein Sec23B | |
| Q9NQW6_C71 | ANLN Actin-binding protein | 3015 |
| | anillin | |
| Q9UBF6_C61 | RNF7 RING-box protein 2 | 3016 |
| P51649_C110 | ALDH5A1 Succinate- | 3017 |
| | semialdehyde dehydrogenase, | |
| | mitochondrial | |
| I3L420_C80 | LSM14A Protein LSM14 | 3018 |
| | homolog A | |
| Q13263_C88 | TRIM28 Transcription | 3019 |
| | intermediary factor 1-beta | |
| Q5VTU8_C19 | ATP5EP2 ATP synthase | 3020 |
| | subunit epsilon-like protein, | |
| | mitochondrial | |
| P06733_C337 | ENO1 Alpha-enolase | 3021 |
| P08559_C94 | PDHA1 Pyruvate | 3022 |
| | dehydrogenase E1 component | |
| | subunit alpha, | |
| Q14166_C642 | TTLL12 Tubulin--tyrosine | 3023 |
| | ligase-like protein 12 | |
| Q9BW92_C506 | TARS2 Threonine--tRNA | 3024 |
| | ligase, mitochondrial | |
| Q12824_C223 | SMARCB1 SWI/SNF-related | 3025 |
| | matrix-associated actin- | |
| | dependent | |
| O00410_C733 | IPO5 Importin-5 | 3026 |
| Q9BXV9_C21 | C14orf142 Uncharacterized | 3027 |
| | protein C14orf142 | |
| Q53GG5_C77 | PDLIM3 PDZ and LIM | 3028 |
| | domain protein 3 | |
| P36776_C858 | LONP1 Lon protease homolog, | 3029 |
| | mitochondrial | |
| Q16531_C652 | DDB1 DNA damage-binding | 3030 |
| | protein 1 | |
| Q96PZ0_C38 | PUS7 Pseudouridylate | 3031 |
| | synthase 7 homolog | |
| Q9HBM6_C121 | TAF9B Transcription initiation | 3032 |
| | factor TFIID subunit 9B | |
| A0AVT1_C625 | UBA6 Ubiquitin-like modifier- | 3033 |
| | activating enzyme 6 | |
| P53384_C31 | NUBP1 Cytosolic Fe—S cluster | 3034 |
| | assembly factor NUBP1 | |
| Q8TBE9_C242 | NANP N-acylneuraminate-9- | 3035 |
| | phosphatase | |
| P10644_C362 | PRKAR1A cAMP-dependent | 3036 |
| | protein kinase type I-alpha | |
| | regulatory subunit alpha | |
| P49790_C1065 | NUP153 Nuclear pore | 3037 |
| | complex protein Nup153 | |
| Q00610_C617 | CLTC Clathrin heavy chain 1 | 3038 |
| Q00610_C753 | CLTC Clathrin heavy chain 1 | 3039 |
| O00506_C357 | STK25 Serine/threonine- | 3040 |
| | protein kinase 25 | |
| Q6SJ93_C523 | FAM111B Protein FAM111B | 3041 |
| Q01581_C86 | HMGCS1 | 3042 |
| | Hydroxymethylglutaryl-CoA | |
| | synthase, cytoplasmic | |
| P35658_C917 | NUP214 Nuclear pore | 3043 |
| | complex protein Nup214 | |
| P46013_C958 | MKI67 Antigen KI-67 | 3044 |
| Q14005_C975 | IL16 Pro-interleukin-16 | 3045 |
| P40937_C73 | RFC5 Replication factor C | 3046 |
| | subunit 5 | |
| Q14653_C371 | IRF3 Interferon regulatory | 3047 |
| | factor 3 | |
| P07900_C420 | HSP90AA1 Heat shock protein | 3048 |
| | HSP 90-alpha | |
| P21333_C649 | FLNA Filamin-A | 3049 |
| P21333_C1260 | FLNA Filamin-A | 3050 |
| P21333_C2160 | FLNA Filamin-A | 3051 |
| Q7Z3D6_C265 | C14orf159 UPF0317 protein | 3052 |
| | C14orf159, mitochondrial | |
| O00264_C129 | PGRMC1 Membrane- | 3053 |
| | associated progesterone | |
| | receptor component 1 | |
| Q15345_C123 | LRRC41 Leucine-rich repeat- | 3054 |
| | containing protein 41 | |
| P43490_C397 | NAMPT Nicotinamide | 3055 |
| | phosphoribosyltransferase | |
| O43504_C66 | HBXIP Hepatitis B virus X- | 3056 |
| | interacting protein | |
| P48307_C130 | TFPI2 Tissue factor pathway | 3057 |
| | inhibitor 2 | |
| P18074_C663 | ERCC2 TFHH basal | 3058 |
| | transcription factor complex | |
| | helicase | |
| Q9NV35_C23 | NUDT15 Probable 8-oxo- | 3059 |
| | dGTP diphosphatase NUDT15 | |
| Q12888_C896 | TP53BP1 Tumor suppressor | 3060 |
| | p53-binding protein 1 | |
| Q8TAC2_C168 | JOSD2 Josephin-2 | 3061 |
| Q6P1L8_C90 | MRPL14 39S ribosomal | 3062 |
| | protein L14, mitochondrial | |
| Q92576_C441 | PHF3 PHD finger protein 3 | 3063 |
| P63000_C105 | RAC1 Ras-related C3 | 3064 |
| | botulinum toxin substrate 1 | |
| O94776_C261 | MTA2 Metastasis-associated | 3065 |
| | protein MTA2 | |
| Q9P0K7_C131 | RAI14 Ankycorbin | 3066 |
| Q92747_C162 | ARPC1A Actin-related protein | 3067 |
| | 2/3 complex subunit 1A | |
| Q8NE71_C741 | ABCF1 ATP-binding cassette | 3068 |
| | sub-family F member 1 | |
| P58546_C45 | MTPN Myotrophin | 3069 |
| Q10570_C1044 | CPSF1 Cleavage and | 3070 |
| | polyadenylation specificity | |
| | factor subunit 1 | |
| Q15021_C320 | NCAPD2 Condensin complex | 3071 |
| | subunit 1 | |
| Q6P1N0_C252 | CC2D1A Coiled-coil and C2 | 3072 |
| | domain-containing protein 1A | |
| O43818_C399 | RRP9 U3 small nucleolar | 3073 |
| | RNA-interacting protein 2 | |
| Q92556_C726 | ELMO1 Engulfment and cell | 3074 |
| | motility protein 1 | |
| Q9NSP4_C110 | CENPM Centromere protein | 3075 |
| | M | |
| P22234_C151 | PAICS Multifunctional protein | 3076 |
| | ADE2 | |
| Q96RN5_C698 | MED15 Mediator of RNA | 3077 |
| | polymerase II transcription | |
| | subunit | |
| Q8IWZ3_C181 | ANKHD1 Ankyrin repeat and | 3078 |
| | KH domain-containing protein | |
| | 1 | |
| H3BN98_C109 | Uncharacterized protein | 3079 |
| Q14683_C1115 | SMC1A Structural | 3080 |
| | maintenance of chromosomes | |
| | protein 1A | |
| P33764_C99 | S100A3 Protein S100-A3 | 3081 |
| Q8NF50_C143 | DOCK8 Dedicator of | 3082 |
| | cytokinesis protein 8 | |
| Q9NZT2_C87 | OGFR Opioid growth factor | 3083 |
| | receptor | |
| Q13620_C248 | CUL4B Cullin-4B | 3084 |
| P00505_C187 | GOT2 Aspartate | 3085 |
| | aminotransferase, | |
| | mitochondrial | |
| Q9Y262_C417 | EIF3L Eukaryotic translation | 3086 |
| | initiation factor 3 subunit | |
| O75521_C282 | ECI2 Enoyl-CoA delta | 3087 |
| | isomerase 2, mitochondrial | |
| Q01469_C120 | FABP5 Fatty acid-binding | 3088 |
| | protein, epidermal | |
| O14965_C33 | AURKA Aurora kinase A | 3089 |
| P24844_C109 | MYL9 Myosin regulatory light | 3090 |
| | polypeptide 9 | |
| Q9ULT8_C2415 | HECTD1 E3 ubiquitin-protein | 3091 |
| | ligase HECTD1 | |
| P81877_C82 | SSBP2 Single-stranded DNA- | 3092 |
| | binding protein 2 | |
| Q9UJX3_C329 | ANAPC7 Anaphase-promoting | 3093 |
| | complex subunit 7 | |
| Q9BW19_C663 | KIFC1 Kinesin-like protein | 3094 |
| | KIFC1 | |
| P51553_C333 | IDH3G Isocitrate | 3095 |
| | dehydrogenase | |
| P32320_C53 | CDA Cytidine deaminase | 3096 |
| Q02750_C376 | MAP2K1 Dual specificity | 3097 |
| | mitogen-activated protein | |
| | kinase | |
| O94829_C163 | IPO13 Importin-13 | 3098 |
| Q9UNH7_C149 | SNX6 Sorting nexin-6 | 3099 |
| Q9UQ80_C149 | PA2G4 Proliferation- | 3100 |
| | associated protein 2G4 | |
| P13674_C503 | P4HA1 Prolyl 4-hydroxylase | 3101 |
| | subunit alpha-1 | |
| O95239_C153 | KIF4A Chromosome- | 3102 |
| | associated kinesin KIF4A | |
| P08134_C20 | RHOC Rho-related GTP- | 3103 |
| | binding protein RhoC | |
| P15121_C45 | AKR1B1 Aldose reductase | 3104 |
| P42704_C208 | LRPPRC Leucine-rich PPR | 3105 |
| | motif-containing protein, | |
| | mitochondrial | |
| Q9BYV9_C370 | BACH2 Transcription | 3106 |
| | regulator protein BACH2 | |
| | SPACPFDK | |
| | R.SPAC*PFDK.G | |
| Q9P0V9_C22 | SEPT10 Septin-10 | 3107 |
| P51570_C203 | GALK1 Galactokinase | 3108 |
| Q7Z5L9_C37 | IRF2BP2 Interferon regulatory | 3109 |
| | factor 2-binding protein 2 | |
| Q86VP6_C1007 | CAND1 Cullin-associated | 3110 |
| | NEDD8-dissociated protein 1 | |
| Q6KB66_C244 | KRT80 Keratin, type II | 3111 |
| | cytoskeletal 80 | |
| P17980_C240 | PSMC3 26S protease | 3112 |
| | regulatory subunit 6A | |
| P13797_C566 | PLS3 Plastin-3 | 3113 |
| Q15599_C271 | SLC9A3R2 Na(+)/H(+) | 3114 |
| | exchange regulatory cofactor | |
| | NHE-RF2 | |
| P41134_C45 | ID1 DNA-binding protein | 3115 |
| | inhibitor ID-1 | |
| Q06210_C264 | GFPT1 Glucosamine-- | 3116 |
| | fructose-6-phosphate | |
| | aminotransferase | |
| P12694_C178 | BCKDHA 2-oxoisovalerate | 3117 |
| | dehydrogenase subunit alpha, | |
| | mitochondrial | |
| Q5XKP0_C60 | QIL1 Protein QIL1 | 3118 |
| Q9UMS4_C298 | PRPF19 Pre-mRNA- | 3119 |
| | processing factor 19 | |
| Q9H2U2_C44 | PPA2 Inorganic | 3120 |
| | pyrophosphatase 2, | |
| | mitochondrial | |
| Q9H2U2_C283 | PPA2 Inorganic | 3121 |
| | pyrophosphatase 2, | |
| | mitochondrial | |
| Q68CZ2_C615 | TNS3 Tensin-3 | 3122 |
| Q99933_C272 | BAG1 BAG family molecular | 3123 |
| | chaperone regulator 1 | |
| Q86U42_C205 | PABPN1 Polyadenylate- | 3124 |
| | binding protein 2 | |
| Q14141_C42 | SEPT6 Septin-6 | 3125 |
| Q9H4B0_C390 | OSGEPL1 Probable tRNA | 3126 |
| | threonylcarbamoyladenosine | |
| | biosynthesis protein OSGEPL1 | |
| P13796_C140 | LCP1 Plastin-2 | 3127 |
| Q06330_C397 | RBPJ Recombining binding | 3128 |
| | protein suppressor of hairless | |
| Q6NVY1_C45 | HIBCH 3-hydroxyisobutyryl- | 3129 |
| | CoA hydrolase, mitochondrial | |
| Q9BYB4_C175 | GNB1L Guanine nucleotide- | 3130 |
| | binding protein subunit beta- | |
| | like protein 1 | |
| P43681_C225 | CHRNA4 Neuronal | 3131 |
| | acetylcholine receptor subunit | |
| | alpha-4 | |
| Q9Y490_C2408 | TLN1 Talin-1 | 3132 |
| P39748_C163 | FEN1 Flap endonuclease 1 | 3133 |
| P49137_C224 | MAPKAPK2 MAP kinase- | 3134 |
| | activated protein kinase 2 | |
| O96013_C276 | PAK4 Serine/threonine-protein | 3135 |
| | kinase PAK 4 | |
| P49748_C477 | ACADVL Very long-chain | 3136 |
| | specific acyl-CoA | |
| | dehydrogenase, m | |
| Q7Z4W1_C244 | DCXR Lxylulose reductase | 3137 |
| P33992_C355 | MCM5 DNA replication | 3138 |
| | licensing factor MCM5 | |
| A6NHR9_C59 | SMCHD1 Structural | 3139 |
| | maintenance of chromosomes | |
| | flexible hinge domain | |
| | containing 1 | |
| O15460_C529 | P4HA2 Prolyl 4-hydroxylase | 3140 |
| | subunit alpha-2 | |
| Q12824_C350 | SMARCB1 SWI/SNF-related | 3141 |
| | matrix-associated actin- | |
| | dependent | |
| O00410_C944 | IPOS Importin-5 | 3142 |
| Q96CP2_C64 | FLYWCH2 FLYWCH family | 3143 |
| | member 2 | |
| Q15080_C84 | NCF4 Neutrophil cytosol | 3144 |
| | factor 4 | |
| Q02241_C483 | KIF23 Kinesin-like protein | 3145 |
| | KIF23 | |
| Q9NSV4_C102 | DIAPH3 Protein diaphanous | 3146 |
| | homolog 3 | |
| Q96N67_C1944 | DOCK7 Dedicator of | 3147 |
| | cytokinesis protein 7 | |
| P16885_C1200 | PLCG2 1-phosphatidylinositol | 3148 |
| | 4,5-bisphosphate | |
| | phosphodiesterase gamma-2 | |
| Q15366_C301 | PCBP2 Poly(rC)-binding | 3149 |
| | protein 2 | |
| Q92598_C290 | HSPH1 Heat shock protein 105 | 3150 |
| | kDa | |
| P53384_C277 | NUBP1 Cytosolic Fe—S cluster | 3151 |
| | assembly factor NUBP1 | |
| Q15181_C254 | PPA1 Inorganic | 3152 |
| | pyrophosphatase | |
| Q7Z5K2_C344 | WAPAL Wings apart-like | 3153 |
| | protein homolog | |
| Q7Z5K2_C906 | WAPAL Wings apart-like | 3154 |
| | protein homolog | |
| Q8NFC6_C285 | BOD1L1 Biorientation of | 3155 |
| | chromosomes in cell division | |
| | protein | |
| P53634_C258 | CTSC Dipeptidyl peptidase 1 | 3156 |
| Q96G25_C31 | MED8 Mediator of RNA | 3157 |
| | polymerase II transcription | |
| | subunit | |
| P10644_C39 | PRKAR1A cAMP-dependent | 3158 |
| | protein kinase type I-alpha | |
| | regulat | |
| O00273_C38 | DFFA DNA fragmentation | 3159 |
| | factor subunit alpha | |
| P49792_C2577 | RANBP2 E3 SUMO-protein | 3160 |
| | ligase RanBP2 | |
| P49792_C2791 | RANBP2 E3 SUMO-protein | 3161 |
| | ligase RanBP2 | |
| Q93009_C961 | USP7 Ubiquitin carboxyl- | 3162 |
| | terminal hydrolase 7 | |
| Q00610_C491 | CLTC Clathrin heavy chain 1 | 3163 |
| O60547_C92 | GMDS GDP-mannose 4,6 | 3164 |
| | dehydratase | |
| Q9H0P0_C73 | NT5C3 Cytosolic 5- | 3165 |
| | nucleotidase 3 | |
| O75427_C105 | LRCH4 Leucine-rich repeat | 3166 |
| | and calponin homology | |
| | domain-containing 4 | |
| Q4G0N4_C58 | NADKD1 NAD kinase | 3167 |
| | domain-containing protein 1 | |
| Q9Y4P8_C393 | WIPI2 WD repeat domain | 3168 |
| | phosphoinositide-interacting | |
| | protein | |
| P27695_C65 | APEX1 DNA-(apurinic or | 3169 |
| | apyrimidinic site) lyase | |
| Q15004_C99 | KIAA0101 PCNA-associated | 3170 |
| | factor | |
| P17858_C114 | PFKL 6-phosphofructokinase, | 3171 |
| | liver type | |
| P17858_C334 | PFKL 6-phosphofructokinase, | 3172 |
| | liver type | |
| Q96PK6_C90 | RBM14 RNA-binding protein | 3173 |
| | 14 | |
| Q09028_C167 | RBBP4 Histone-binding | 3174 |
| | protein RBBP4 | |
| Q8TC07_C24 | TBC1D15 TBC1 domain | 3175 |
| | family member 15 | |
| Q7Z6Z7_C2721 | HUWE1 E3 ubiquitin-protein | 3176 |
| | ligase HUWE1 | |
| Q9H6S3_C369 | EPS8L2 Epidermal growth | 3177 |
| | factor receptor kinase substrate | |
| Q9Y281_C80 | CFL2 Cofilin-2 | 3178 |
| Q96IZ6_C171 | METTL2A Methyltransferase- | 3179 |
| | like protein 2A | |
| Q96EM0_C39 | L3HYPDH Trans-L-3- | 3180 |
| | hydroxyproline dehydratase | |
| Q92616_C2255 | GCN1L1 Translational | 3181 |
| | activator GCN1 | |
| P43490_C401 | NAMPT Nicotinamide | 3182 |
| | phosphoribosyltransferase | |
| P22087_C268 | FBL rRNA 2-O- | 3183 |
| | methyltransferase fibrillarin | |
| Q92503_C644 | SEC14L1 SEc14-like protein | 3184 |
| | 1 | |
| Q9UI10_C69 | EIF2B4 Translation initiation | 3185 |
| | factor eIF-2B subunit delta | |
| P49327_C161 | FASN Fatty acid synthase | 3186 |
| P49327_C1141 | FASN Fatty acid synthase | 3187 |
| P49327_C1227 | FASN Fatty acid synthase | 3188 |
| Q9ULZ3_C173 | PYCARD Apoptosis- | 3189 |
| | associated speck-like protein | |
| | containing | |
| Q8WUI4_C904 | HDAC7 Histone deacetylase 7 | 3190 |
| Q7L2J0_C324 | MEPCE 7SK snRNA | 3191 |
| | methylphosphate capping | |
| | enzyme | |
| Q9BUK6_C46 | MSTO1 Protein misato | 3192 |
| | homolog 1 | |
| P54577_C424 | YARS Tyrosine--tRNA ligase, | 3193 |
| | cytoplasmic | |
| Q8WU90_C105 | ZC3H15 Zinc finger CCCH | 3194 |
| | domain-containing protein 15 | |
| Q9Y305_C194 | ACOT9 Acyl-coenzyme A | 3195 |
| | thioesterase 9, mitochondrial | |
| Q15149_C1098 | PLEC Plectin | 3196 |
| Q14669_C535 | TRIP12 E3 ubiquitin-protein | 3197 |
| | ligase TRIP12 | |
| Q9BTT0_C123 | ANP32E Acidic leucine-rich | 3198 |
| | nuclear phosphoprotein 32 | |
| | family member E | |
| Q92989_C338 | CLP1 Polyribonucleotide 5- | 3199 |
| | hydroxyl-kinase Clp1 | |
| P06132_C59 | UROD Uroporphyrinogen | 3200 |
| | decarboxylase | |
| Q96EY7_C139 | PTCD3 Pentatricopeptide | 3201 |
| | repeat-containing protein 3, | |
| | mitochondrial | |
| P78527_C90 | PRKDC DNA-dependent | 3202 |
| | protein kinase catalytic subunit | |
| P78527_C1312 | PRKDC DNA-dependent | 3203 |
| | protein kinase catalytic subunit | |
| P78527_C1767 | PRKDC DNA-dependent | 3204 |
| | protein kinase catalytic subunit | |
| Q92747_C279 | ARPC1A Actin-related protein | 3205 |
| | 2/3 complex subunit 1A | |
| Q14671_C234 | PUM1 Pumilio homolog 1 | 3206 |
| O60216_C585 | RAD21 Double-strand-break | 3207 |
| | repair protein rad21 homolog | |
| Q5VZF2_C19 | MBNL2 Muscleblind-like | 3208 |
| | protein 2 | |
| Q9UEW8_C525 | STK39 STE20/SPS1-related | 3209 |
| | proline-alanine-rich protein | |
| | kinase | |
| O00232_C255 | PSMD12 26S proteasome non- | 3210 |
| | ATPase regulatory subunit 12 | |
| O00233_C81 | PSMD9 26S proteasome non- | 3211 |
| | ATPase regulatory subunit 9 | |
| Q5GLZ8_C392 | HERC4 Probable E3 ubiquitin- | 3212 |
| | protein ligase HERC4 | |
| O14579_C212 | COPE Coatomer subunit | 3213 |
| | epsilon | |
| Q7Z4G1_C35 | COMMD6 COMM domain- | 3214 |
| | containing protein 6 | |
| P17812_C218 | CTPS1 CTP synthase 1 | 3215 |
| O95394_C200 | PGM3 | 3216 |
| | Phosphoacetylglucosamine | |
| | mutase | |
| Q7LBC6_C1357 | KDM3B Lysine-specific | 3217 |
| | demethylase 3B | |
| Q99496_C72 | RNF2 E3 ubiquitin-protein | 3218 |
| | ligase RING2 | |
| Q9UNW1_C232 | MINPP1 Multiple inositol | 3219 |
| | polyphosphate phosphatase 1 | |
| P50453_C259 | SERPINB9 Serpin B9 | 3220 |
| Q9NZT2_C417 | OGFR Opioid growth factor | 3221 |
| | receptor | |
| O95573_C450 | ACSL3 Long-chain-fatty-acid-- | 3222 |
| | CoA ligase 3 | |
| O60841_C720 | EIF5B Eukaryotic translation | 3223 |
| | initiation factor 5B | |
| P29317_C612 | EPHA2 Ephrin type-A | 3224 |
| | receptor 2 | |
| O14561_C140 | NDUFAB1 Acyl carrier | 3225 |
| | protein, mitochondrial | |
| Q9NXH9_C639 | TRMT1 tRNA (guanine(26)- | 3226 |
| | N(2))-dimethyltransferase | |
| Q9H6Y2_C306 | WDR55 WD repeat-containing | 3227 |
| | protein 55 | |
| A0JLT2_C163 | MED19 Mediator of RNA | 3228 |
| | polymerase II transcription | |
| | subunit | |
| Q10567_C565 | AP1B1 AP-1 complex subunit | 3229 |
| | beta-1 | |
| P08243_C115 | ASNS Asparagine synthetase | 3230 |
| P08243_C158 | ASNS Asparagine synthetase | 3231 |
| P46736_C273 | BRCC3 Lys-63-specific | 3232 |
| | deubiquitinase BRCC36 | |
| Q01469_C67 | FABP5 Fatty acid-binding | 3233 |
| | protein, epidermal | |
| O43175_C116 | PHGDH D-3- | 3234 |
| | phosphoglycerate | |
| | dehydrogenase | |
| O14818_C91 | PSMA7 Proteasome subunit | 3235 |
| | alpha type-7 | |
| Q9BRA2_C110 | TXNDC17 Thioredoxin | 3236 |
| | domain-containing protein 17 | |
| P01892_C363 | HLA-A HLA class I histocompatibility | 3237 |
| | antigen, A-2 alpha | |
| P84085_C159 | ARF5 ADP-ribosylation factor | 3238 |
| | 5 | |
| P62879_C204 | GNB2 Guanine nucleotide- | 3239 |
| | binding protein G(I)/G(S)/G(T) | |
| Q9C0C9_C244 | UBE2O Ubiquitin-conjugating | 3240 |
| | enzyme E2 O | |
| Q9C0C9_C598 | UBE2O Ubiquitin-conjugating | 3241 |
| | enzyme E2 O | |
| Q14204_C3573 | DYNC1H1 Cytoplasmic | 3242 |
| | dynein 1 heavy chain 1 | |
| O60684_C238 | KPNA6 Importin subunit | 3243 |
| | alpha-7 | |
| P20073_C413 | ANXA7 Annexin A7 | 3244 |
| O75643_C1127 | SNRNP200 U5 small nuclear | 3245 |
| | ribonucleoprotein 200 kDa | |
| | helicase | |
| P32320_C31 | CDA Cytidine deaminase | 3246 |
| P11586_C236 | MTHFD1 C-1-tetrahydrofolate | 3247 |
| | synthase, cytoplasmic | |
| H3BN57_C113 | BLOC1S5-TXNDC5 Protein | 3248 |
| | BLOC1S5-TXNDC5 | |
| Q53HC9_C95 | TSSC1 Protein TSSC1 | 3249 |
| P27816_C67 | MAP4 Microtubule-associated | 3250 |
| | protein 4 | |
| P84090_C33 | ERH Enhancer of rudimentary | 3251 |
| | homolog | |
| O00487_C299 | PSMD14 26S proteasome non- | 3252 |
| | ATPase regulatory subunit 14 | |
| Q8N8R5_C43 | C2orf69 UPF0565 protein | 3253 |
| | C2orf69 | |
| P52948_C1027 | NUP98 Nuclear pore complex | 3254 |
| | protein Nup98-Nup96 | |
| P52948_C1068 | NUP98 Nuclear pore complex | 3255 |
| | protein Nup98-Nup96 | |
| Q4G0P3_C1853 | HYDIN Hydrocephalus- | 3256 |
| | inducing protein homolog | |
| Q08J23_C502 | NSUN2 tRNA (cytosine(34)- | 3257 |
| | C(5))-methyltransferase | |
| O15231_C542 | ZNF185 Zinc finger protein | 3258 |
| | 185 | |
| P21291_C122 | CSRP1 Cysteine and glycine- | 3259 |
| | rich protein 1 | |
| O43447_C122 | PPIH Peptidyl-prolyl cis-trans | 3260 |
| | isomerase H | |
| P08237_C170 | PFKM 6-phosphofructokinase, | 3261 |
| | muscle type | |
| Q1KMD3_C602 | HNRNPUL2 Heterogeneous | 3262 |
| | nuclear ribonucleoprotein U- | |
| | like protein 2 | |
| P51991_C94 | HNRNPA3 Heterogeneous | 3263 |
| | nuclear ribonucleoprotein A3 | |
| P35237_C36 | SERPINB6 Serpin B6 | 3264 |
| Q96FJ2_C24 | DYNLL2 Dynein light chain 2, | 3265 |
| | cytoplasmic | |
| Q9BTZ2_C209 | DHRS4 | 3266 |
| | Dehydrogenase/reductase SDR | |
| | family member 4 | |
| Q9UMS4_C230 | PRPF19 Pre-mRNA- | 3267 |
| | processing factor 19 | |
| Q9Y6A5_C459 | TACC3 Transforming acidic | 3268 |
| | coiled-coil-containing protein | |
| Q86TI0_C604 | TBC1D1 TBC1 domain family | 3269 |
| | member 1 | |
| Q9H0W9_C226 | C11orf54 Ester hydrolase | 3270 |
| | C11orf54 | |
| P18031_C121 | PTPN1 Tyrosine-protein | 3271 |
| | phosphatase non-receptor type | |
| | 1 | |
| O60343_C1286 | TBC1D4 TBC1 domain family | 3272 |
| | member 4 | |
| O60759_C210 | CYTIP Cytohesin-interacting | 3273 |
| | protein | |
| P10398_C192 | ARAF Serine/threonine- | 3274 |
| | protein kinase A-Raf | |
| Q71SY5_C111 | MED25 Mediator of RNA | 3275 |
| | polymerase II transcription | |
| | subunit | |
| Q9Y490_C1392 | TLN1 Talin-1 | 3276 |
| P43246_C873 | MSH2 DNA mismatch repair | 3277 |
| | protein Msh2 | |
| P23381_C305 | WARS Tryptophan--tRNA | 3278 |
| | ligase, cytoplasmic | |
| Q9NXV2_C28 | KCTD5 BTB/POZ domain- | 3279 |
| | containing protein KCTD5 | |
| P21399_C369 | ACO1 Cytoplasmic aconitate | 3280 |
| | hydratase | |
| Q06830_C52 | PRDX1 Peroxiredoxin-1 | 3281 |
| Q96D46_C20 | NMD3 60S ribosomal export | 3282 |
| | protein NMD3 | |
| Q9Y6C9_C79 | MTCH2 Mitochondrial carrier | 3283 |
| | homolog 2 | |
| O00429_C446 | DNM1L Dynamin-1-like | 3284 |
| | protein | |
| P50552_C64 | VASP Vasodilator-stimulated | 3285 |
| | phosphoprotein | |
| Q00796_C120 | SORD Sorbitol dehydrogenase | 3286 |
| P42126_C114 | ECI1 Enoyl-CoA delta | 3287 |
| | isomerase 1, mitochondrial | |
| P49368_C213 | CCT3 T-complex protein 1 | 3288 |
| | subunit gamma | |
| Q9NY27_C105 | PPP4R2 Serine/threonine- | 3289 |
| | protein phosphatase 4 | |
| | regulatory | |
| P49756_C83 | RBM25 RNA-binding protein | 3290 |
| | 25 | |
| F5H5P2_C231 | Uncharacterized protein | 3291 |
| Q14C86_C741 | GAPVD1 GTPase-activating | 3292 |
| | protein and VPS9 domain- | |
| | containing protein 1 | |
| Q969V6_C326 | MKL1 MKL/myocardin-like | 3293 |
| | protein 1 | |
| Q9P2N5_C21 | RBM27 RNA-binding protein | 3294 |
| | 27 | |
| P53396_C229 | ACLY ATP-citrate synthase | 3295 |
| Q8WVT3_C160 | TRAPPC12 Trafficking | 3296 |
| | protein particle complex | |
| | subunit 12 | |
| O43678_C24 | NDUFA2 NADH | 3297 |
| | dehydrogenase | |
| Q96IF1_C270 | AJUBA LIM domain- | 3298 |
| | containing protein ajuba | |
| Q14974_C158 | KPNB1 Importin subunit beta- | 3299 |
| | 1 | |
| P21266_C178 | GSTM3 Glutathione S- | 3300 |
| | transferase Mu 3 | |
| O00622_C322 | CYR61 Protein CYR61 | 3301 |
| Q96N67_C193 | DOCK7 Dedicator of | 3302 |
| | cytokinesis protein 7 | |
| Q15369_C74 | TCEB1 Transcription | 3303 |
| | elongation factor B | |
| | polypeptide 1 | |
| A6NDU8_C57 | C5orf51 UPF0600 protein | 3304 |
| | C5orf51 | |
| Q9P219_C1871 | CCDC88C Protein Daple | 3305 |
| Q09161_C73 | NCBP1 Nuclear cap-binding | 3306 |
| | protein subunit 1 | |
| Q69YN2_C141 | CWF19L1 CWF19-like protein | 3307 |
| | 1 | |
| Q70E73_C1045 | RAPH1 Ras-associated and | 3308 |
| | pleckstrin homology domains- | |
| | containing protein 1 | |
| Q93008_C1237 | USP9X Probable ubiquitin | 3309 |
| | carboxyl-terminal hydrolase | |
| | FAF | |
| Q9UQ13_C144 | SHOC2 Leucine-rich repeat | 3310 |
| | protein SHOC-2 | |
| Q9BXP5_C715 | SRRT Serrate RNA effector | 3311 |
| | molecule homolog | |
| Q96P65_C201 | QRFPR Pyroglutamylated | 3312 |
| | RFamide peptide receptor | |
| P46013_C261 | MKI67 Antigen KI-67 | 3313 |
| P17858_C630 | PFKL 6-phosphofructokinase, | 3314 |
| | liver type | |
| O75083_C285 | WDR1 WD repeat-containing | 3315 |
| | protein 1 | |
| O43252_C78 | PAPSS1 Bifunctional 3- | 3316 |
| | phosphoadenosine 5- | |
| | phosphosulfate | |
| Q6WCQ1_C724 | MPRIP Myosin phosphatase | 3317 |
| | Rho-interacting protein | |
| Q14008_C1360 | CKAP5 Cytoskeleton- | 3318 |
| | associated protein 5 | |
| P40939_C349 | HADHA Trifunctional enzyme | 3319 |
| | subunit alpha, mitochondrial | |
| P21333_C2293 | FLNA Filamin-A | 3320 |
| P30566_C483 | ADSL Adenylosuccinate lyase | 3321 |
| Q9Y285_C493 | FARSA Phenylalanine--tRNA | 3322 |
| | ligase alpha subunit | |
| Q06124_C318 | PTPN11 Tyrosine-protein | 3323 |
| | phosphatase non-receptor type | |
| | 11 | |
| Q96SZ5_C18 | ADO 2-aminoethanethiol | 3324 |
| | dioxygenase | |
| Q96CM8_C122 | ACSF2 Acyl-CoA synthetase | 3325 |
| | family member 2, | |
| | mitochondrial | |
| P46063_C606 | RECQL ATP-dependent DNA | 3326 |
| | helicase Q1 | |
| P53618_C248 | COPB1 Coatomer subunit beta | 3327 |
| P53618_C684 | COPB1 Coatomer subunit beta | 3328 |
| Q9UI10_C465 | EIF2B4 Translation initiation | 3329 |
| | factor eIF-2B subunit delta | |
| Q9UI10_C509 | EIF2B4 Translation initiation | 3330 |
| | factor eIF-2B subunit delta | |
| Q8WV28_C271 | BLNK B-cell linker protein | 3331 |
| P49327_C2273 | FASN Fatty acid synthase | 3332 |
| Q9UGV2_C359 | NDRG3 Protein NDRG3 | 3333 |
| Q99436_C87 | PSMB7 Proteasome subunit | 3334 |
| | beta type-7 | |
| Q86YS7_C867 | KIAA0528 Uncharacterized | 3335 |
| | protein KIAA0528 | |
| P48444_C286 | ARCN1 Coatomer subunit | 3336 |
| | delta | |
| Q86V48_C560 | LUZP1 Leucine zipper protein | 3337 |
| | 1 | |
| Q9UQ35_C1029 | SRRM2 Serine/arginine | 3338 |
| | repetitive matrix protein 2 | |
| Q9BSH5_C109 | HDHD3 Haloacid | 3339 |
| | dehalogenase-like hydrolase | |
| | domain-containing 3 | |
| Q92973_C790 | TNPO1 Transportin-1 | 3340 |
| Q8N3F8_C597 | MICALL1 MICAL-like | 3341 |
| | protein 1 | |
| O95425_C1949 | SVIL Supervillin | 3342 |
| Q8IVH2_C447 | FOXP4 Forkhead box protein | 3343 |
| | P4 | |
| Q15149_C965 | PLEC Plectin | 3344 |
| P25788_C42 | PSMA3 Proteasome subunit | 3345 |
| | alpha type-3 | |
| Q9HB40_C152 | SCPEP1 Retinoid-inducible | 3346 |
| | serine carboxypeptidase | |
| Q08945_C139 | SSRP1 FACT complex subunit | 3347 |
| | SSRP1 | |
| Q00577_C272 | PURA Transcriptional | 3348 |
| | activator protein Pur-alpha | |
| Q96EY5_C33 | FAM125A Multivesicular | 3349 |
| | body subunit 12A | |
| P13639_C536 | EEF2 Elongation factor 2 | 3350 |
| P28799_C178 | GRN Granulins | 3351 |
| Q14258_C475 | TRIM25 E3 ubiquitin/ISG15 | 3352 |
| | ligase TRIM25 | |
| O75694_C874 | NUP155 Nuclear pore | 3353 |
| | complex protein Nup155 | |
| P49189_C267 | ALDH9A1 4- | 3354 |
| | trimethylaminobutyraldehyde | |
| | dehydrogenase | |
| O43592_C522 | XPOT Exportin-T | 3355 |
| Q9NUQ3_C127 | TXLNG Gamma-taxilin | 3356 |
| O94760_C178 | DDAH1 N(G),N(G)- | 3357 |
| | dimethylarginine | |
| | dimethylaminohydrolase | |
| P61163_C222 | ACTR1A Alpha-centractin | 3358 |
| Q12905_C311 | ILF2 Interleukin enhancer- | 3359 |
| | binding factor 2 | |
| Q14814_C217 | MEF2D Myocyte-specific | 3360 |
| | enhancer factor 2D | |
| P50990_C430 | CCT8 T-complex protein 1 | 3361 |
| | subunit theta | |
| P49848_C644 | TAF6 Transcription initiation | 3362 |
| | factor TFIID subunit 6 | |
| O00541_C153 | PES1 Pescadillo homolog | 3363 |
| P17812_C30 | CTPS1 CTP synthase 1 | 3364 |
| Q9P287_C213 | BCCIP BRCA2 and | 3365 |
| | CDKN1A-interacting protein | |
| Q7LBC6_C1212 | KDM3B Lysine-specific | 3366 |
| | demethylase 3B | |
| Q9Y3P9_C155 | RABGAP1 Rab GTPase- | 3367 |
| | activating protein 1 | |
| Q99497_C53 | PARK7 Protein DJ-1 | 3368 |
| O95571_C98 | ETHE1 Protein ETHE1, | 3369 |
| | mitochondrial | |
| Q8WUM0_C530 | NUP133 Nuclear pore | 3370 |
| | complex protein Nup133 | |
| P11387_C630 | TOP1 DNA topoisomerase 1 | 3371 |
| P25098_C340 | ADRBK1 Beta-adrenergic | 3372 |
| | receptor kinase 1 | |
| Q15796_C70 | SMAD2 Mothers against | 3373 |
| | decapentaplegic homolog 2 | |
| Q8NF50_C170 | DOCK8 Dedicator of | 3374 |
| | cytokinesis protein 8 | |
| Q8NF50_C186 | DOCK8 Dedicator of | 3375 |
| | cytokinesis protein 8 | |
| Q8N5N7_C52 | MRPL50 39S ribosomal | 3376 |
| | protein L50, mitochondrial | |
| Q86UW9_C351 | DTX2 Probable E3 ubiquitin- | 3377 |
| | protein ligase DTX2 | |
| I3L3Q1_C657 | Uncharacterized protein | 3378 |
| Q96QA5_C174 | GSDMA Gasdermin-A | 3379 |
| Q9Y263_C605 | PLAA Phospholipase A-2- | 3380 |
| | activating protein | |
| O75521_C312 | ECI2 Enoyl-CoA delta | 3381 |
| | isomerase 2, mitochondrial | |
| P08243_C44 | ASNS Asparagine synthetase | 3382 |
| Q9BQS8_C293 | FYCO1 FYVE and coiled-coil | 3383 |
| | domain-containing protein 1 | |
| Q96QK1_C699 | VPS35 Vacuolar protein | 3384 |
| | sorting-associated protein 35 | |
| Q8NDI1_C849 | EHBP1 EH domain-binding | 3385 |
| | protein 1 | |
| P19367_C158 | HK1 Hexokinase-1 | 3386 |
| O14818_C63 | PSMA7 Proteasome subunit | 3387 |
| | alpha type-7 | |
| Q13439_C1085 | GOLGA4 Golgin subfamily A | 3388 |
| | member 4 | |
| P63279_C93 | UBE2I SUMO-conjugating | 3389 |
| | enzyme UBC9 | |
| Q15003_C114 | NCAPH Condensin complex | 3390 |
| | subunit 2 | |
| O75534_C730 | CSDE1 Cold shock domain- | 3391 |
| | containing protein E1 | |
| Q8WVB6_C373 | CHTF18 Chromosome | 3392 |
| | transmission fidelity protein 18 | |
| | homolog | |
| Q14204_C3033 | DYNC1H1 Cytoplasmic | 3393 |
| | dynein 1 heavy chain 1 | |
| Q9UN37_C233 | VPS4A Vacuolar protein | 3394 |
| | sorting-associated protein 4A | |
| Q8WV99_C171 | ZFAND2B AN1-type zinc | 3395 |
| | finger protein 2B | |
| P22102_C237 | GART Trifunctional purine | 3396 |
| | biosynthetic protein adenosine | |
| O75643_C133 | SNRNP200 U5 small nuclear | 3397 |
| | ribonucleoprotein 200 kDa | |
| | helicase | |
| P32322_C95 | PYCR1 Pyrroline-5- | 3398 |
| | carboxylate reductase 1, | |
| | mitochondrial | |
| Q9H1K1_C138 | ISCU Iron-sulfur cluster | 3399 |
| | assembly enzyme ISCU, | |
| | mitochondrial | |
| Q6IA69_C428 | NADSYN1 Glutamine- | 3400 |
| | dependent NAD(+) synthetase | |
| Q9UQ80_C296 | PA2G4 Proliferation- | 3401 |
| | associated protein 2G4 | |
| Q9NP81_C64 | SARS2 Serine--tRNA ligase, | 3402 |
| | mitochondrial | |
| Q6L8Q7_C180 | PDE12 2,5-phosphodiesterase | 3403 |
| | 12 | |
| Q15102_C205 | PAFAH1B3 Platelet-activating | 3404 |
| | factor acetylhydrolase IB | |
| | subunit | |
| Q9Y448_C296 | SKAP Small kinetochore- | 3405 |
| | associated protein | |
| Q01082_C1389 | SPTBN1 Spectrin beta chain, | 3406 |
| | non-erythrocytic 1 | |
| P42704_C113 | LRPPRC Leucine-rich PPR | 3407 |
| | motif-containing protein, | |
| | mitochondrial | |
| O60784_C415 | TOM1 Target of Myb protein | 3408 |
| | 1 | |
| P42858_C1998 | HTT Huntingtin | 3409 |
| A2A288_C517 | ZC3H12D Probable | 3410 |
| | ribonuclease ZC3H12D | |
| P31930_C453 | UQCRC1 Cytochrome b-c1 | 3411 |
| | complex subunit 1, | |
| | mitochondrial | |
| P31939_C287 | ATIC Bifunctional purine | 3412 |
| | biosynthesis protein PURH | |
| Q14980_C2009 | NUMA1 Nuclear mitotic | 3413 |
| | apparatus protein 1 | |
| Q9NSE4_C364 | IARS2 Isoleucine--tRNA | 3414 |
| | ligase, mitochondrial | |
| Q9NSE4_C521 | IARS2 Isoleucine--tRNA | 3415 |
| | ligase, mitochondrial | |
| Q96ER3_C248 | SAAL1 Protein SAAL1 | 3416 |
| Q5M775_C493 | SPECC1 Cytospin-B | 3417 |
| Q9P2J5_C446 | LARS Leucine--tRNA ligase, | 3418 |
| | cytoplasmic | |
| P35236_C204 | PTPN7 Tyrosine-protein | 3419 |
| | phosphatase non-receptor type | |
| | 7 | |
| P14324_C204 | FDPS Farnesyl pyrophosphate | 3420 |
| | synthase | |
| P53597_C142 | SUCLG1 Succinyl-CoA ligase | 3421 |
| Q9GZT3_C48 | SLIRP SRA stem-loop- | 3422 |
| | interacting RNA-binding | |
| | protein, mitochondrial | |
| P10619_C403 | CTSA Lysosomal protective | 3423 |
| | protein | |
| Q9UDY2_C601 | TJP2 Tight junction protein | 3424 |
| | ZO-2 | |
| Q13501_C290 | SQSTM1 Sequestosome-1 | 3425 |
| Q53EL6_C150 | PDCD4 Programmed cell | 3426 |
| | death protein 4 | |
| Q13618_C636 | CUL3 Cullin-3 | 3427 |
| Q93052_C465 | LPP Lipoma-preferred partner | 3428 |
| Q92900_C683 | UPF1 Regulator of nonsense | 3429 |
| | transcripts 1 | |
| Q9Y5P6_C230 | GMPPB Mannose-1-phosphate | 3430 |
| | guanyltransferase beta | |
| O14981_C936 | BTAF1 TATA-binding | 3431 |
| | protein-associated factor 172 | |
| Q9NR31_C102 | SAR1A GTP-binding protein | 3432 |
| | SAR1a | |
| Q92623_C30 | TTC9 Tetratricopeptide repeat | 3433 |
| | protein 9A | |
| P41743_C595 | PRKCI Protein kinase C iota | 3434 |
| | type | |
| Q6NVY1_C163 | HIBCH 3-hydroxyisobutyryl- | 3435 |
| | CoA hydrolase, mitochondrial | |
| Q29RF7_C14 | PDS5A Sister chromatid | 3436 |
| | cohesion protein PDS5 | |
| | homolog A | |
| Q9NQW6_C819 | ANLN Actin-binding protein | 3437 |
| | anillin | |
| Q9Y490_C1434 | TLN1 Talin-1 | 3438 |
| Q86W50_C432 | METTL16 Methyltransferase- | 3439 |
| | like protein 16 | |
| Q8IV48_C75 | ERI1 3-5 exoribonuclease 1 | 3440 |
| Q9Y3Z3_C341 | SAMHD1 SAM domain and | 3441 |
| | HD domain-containing protein | |
| | 1 | |
| P38646_C366 | HSPA9 Stress-70 protein, | 3442 |
| | mitochondrial | |
| P47755_C111 | CAPZA2 F-actin-capping | 3443 |
| | protein subunit alpha-2 | |
| P06730_C170 | EIF4E Eukaryotic translation | 3444 |
| | initiation factor 4E | |
| Q9NR09_C1517 | BIRC6 Baculoviral IAP | 3445 |
| | repeat-containing protein 6 | |
| P00491_C78 | PNP Purine nucleoside | 3446 |
| | phosphorylase | |
| P08397_C261 | HMBS Porphobilinogen | 3447 |
| | deaminase | |
| Q7KZF4_C736 | SND1 Staphylococcal nuclease | 3448 |
| | domain-containing protein | |
| O60292_C235 | SIPA1L3 Signal-induced | 3449 |
| | proliferation-associated 1-like | |
| | protein 3 | |
| Q14166_C528 | TTLL12 Tubulin--tyrosine | 3450 |
| | ligase-like protein 12 | |
| Q14166_C572 | TTLL12 Tubulin--tyrosine | 3451 |
| | ligase-like protein 12 | |
| Q13257_C106 | MAD2L1 Mitotic spindle | 3452 |
| | assembly checkpoint protein | |
| | MAD2A | |
| Q9NY27_C134 | PPP4R2 Serine/threonine- | 3453 |
| | protein phosphatase 4 | |
| | regulatory | |
| P30405_C104 | PPIF Peptidyl-prolyl cis-trans | 3454 |
| | isomerase F, mitochondrial | |
| Q9H0D6_C296 | XRN2 5-3 exoribonuclease 2 | 3455 |
| Q8TDG2_C182 | ACTRT1 Actin-related protein | 3456 |
| | T1 | |
| O00410_C473 | IPO5 Importin-5 | 3457 |
| Q5VUA4_C2168 | ZNF318 Zinc finger protein | 3458 |
| | 318 | |
| Q8IXB1_C700 | DNAJC10 DnaJ homolog | 3459 |
| | subfamily C member 10 | |
| P53621_C975 | COPA Coatomer subunit alpha | 3460 |
| P00441_C147 | SOD1 Superoxide dismutase | 3461 |
| Q00534_C306 | CDK6 Cyclin-dependent | 3462 |
| | kinase 6 | |
| P02545_C570 | LMNA Prelamin-A/C | 3463 |
| P16885_C791 | PLCG2 1-phosphatidylinositol | 3464 |
| | 4,5-bisphosphate | |
| | phosphodiesterase gamma-2 | |
| A0AVT1_C546 | UBA6 Ubiquitin-like modifier- | 3465 |
| | activating enzyme 6 | |
| Q15369_C11 | TCEB1 Transcription | 3466 |
| | elongation factor B | |
| | polypeptide 1 | |
| Q9BVL2_C252 | NUPL1 Nucleoporin p58/p45 | 3467 |
| O15091_C367 | KIAA0391 Mitochondrial | 3468 |
| | ribonuclease P protein 3 | |
| Q92835_C902 | INPP5D Phosphatidylinositol | 3469 |
| | 3,4,5-trisphosphate 5- | |
| | phosphatase 1 | |
| Q92835_C1088 | INPP5D Phosphatidylinositol | 3470 |
| | 3,4,5-trisphosphate 5- | |
| | phosphatase 1 | |
| Q9H3M7_C36 | TXNIP Thioredoxin- | 3471 |
| | interacting protein | |
| Q9H3M7_C333 | TXNIP Thioredoxin- | 3472 |
| | interacting protein | |
| Q92598_C845 | HSPH1 Heat shock protein 105 | 3473 |
| | kDa | |
| Q8NEM2_C612 | SHCBP1 SHC SH2 domain- | 3474 |
| | binding protein 1 | |
| Q92619_C324 | HMHA1 Minor | 3475 |
| | histocompatibility protein HA- | |
| | 1 | |
| O94906_C913 | PRPF6 Pre-mRNA-processing | 3476 |
| | factor 6 | |
| Q5TA50_C163 | GLTPD1 Glycolipid transfer | 3477 |
| | protein domain-containing | |
| | protein 1 | |
| Q9UPN7_C172 | PPP6R1 Serine/threonine- | 3478 |
| | protein phosphatase 6 | |
| | regulatory | |
| Q9P289_C392 | MST4 Serine/threonine-protein | 3479 |
| | kinase MST4 | |
| Q16270_C87 | IGFBP7 Insulin-like growth | 3480 |
| | factor-binding protein 7 | |
| P48556_C112 | PSMD8 26S proteasome non- | 3481 |
| | ATPase regulatory subunit 8 | |
| P49792_C1196 | RANBP2 E3 SUMO-protein | 3482 |
| | ligase RanBP2 | |
| P49792_C2407 | RANBP2 E3 SUMO-protein | 3483 |
| | ligase RanBP2 | |
| Q93008_C1212 | USP9X Probable ubiquitin | 3484 |
| | carboxyl-terminal hydrolase | |
| | FAF | |
| Q9Y617_C98 | PSAT1 Phosphoserine | 3485 |
| | aminotransferase | |
| Q9Y617_C152 | PSAT1 Phosphoserine | 3486 |
| | aminotransferase | |
| Q96AB3_C84 | ISOC2 Isochorismatase | 3487 |
| | domain-containing protein 2, | |
| | mitochondrial | |
| Q9H7Z6_C416 | KAT8 Histone | 3488 |
| | acetyltransferase KAT8 | |
| Q01581_C224 | HMGCS1 | 3489 |
| | Hydroxymethylglutaryl-CoA | |
| | synthase, cytoplasmic | |
| P62633_C57 | CNBP Cellular nucleic acid- | 3490 |
| | binding protein | |
| P39023_C114 | RPL3 60S ribosomal protein | 3491 |
| | L3 | |
| P21333_C210 | FLNA Filamin-A | 3492 |
| P21333_C1353 | FLNA Filamin-A | 3493 |
| O95347_C585 | SMC2 Structural maintenance | 3494 |
| | of chromosomes protein 2 | |
| O00469_C562 | PLOD2 Procollagen-lysine,2- | 3495 |
| | oxoglutarate 5-dioxygenase 2 | |
| P45880_C13 | VDAC2 Voltage-dependent | 3496 |
| | anion-selective channel protein | |
| Q96CM8_C462 | ACSF2 Acyl-CoA synthetase | 3497 |
| | family member 2, | |
| | mitochondrial | |
| P18583_C92 | SON Protein SON | 3498 |
| Q9UKX7_C165 | NUP50 Nuclear pore complex | 3499 |
| | protein Nup50 | |
| Q9UKX7_C416 | NUP50 Nuclear pore complex | 3500 |
| | protein Nup50 | |
| Q99661_C287 | KIF2C Kinesin-like protein | 3501 |
| | KIF2C | |
| P42765_C179 | ACAA2 3-ketoacyl-CoA | 3502 |
| | thiolase, mitochondrial | |
| P49327_C779 | FASN Fatty acid synthase | 3503 |
| P49327_C1828 | FASN Fatty acid synthase | 3504 |
| O75347_C67 | TBCA Tubulin-specific | 3505 |
| | chaperone A | |
| P63010_C95 | AP2B1 AP-2 complex subunit | 3506 |
| | beta | |
| Q9UDR5_C87 | AASS Alpha-aminoadipic | 3507 |
| | semialdehyde synthase, | |
| | mitochondrial | |
| P40925_C154 | MDH1 Malate dehydrogenase, | 3508 |
| | cytoplasmic | |
| Q13363_C38 | CTBP1 C-terminal-binding | 3509 |
| | protein 1 | |
| P41091_C311 | EIF2S3 Eukaryotic translation | 3510 |
| | initiation factor 2 subunit | |
| Q9UKI8_C81 | TLK1 Serine/threonine-protein | 3511 |
| | kinase tousled-like 1 | |
| Q13523_C962 | PRPF4B Serine/threonine- | 3512 |
| | protein kinase PRP4 homolog | |
| Q12768_C385 | KIAA0196 WASH complex | 3513 |
| | subunit strumpellin | |
| Q14203_C636 | DCTN1 Dynactin subunit 1 | 3514 |
| Q96RS6_C111 | NUDCD1 NudC domain- | 3515 |
| | containing protein 1 | |
| Q7L2J0_C153 | MEPCE 7SK snRNA | 3516 |
| | methylphosphate capping | |
| | enzyme | |
| Q8N0Z6_C439 | TTC5 Tetratricopeptide repeat | 3517 |
| | protein 5 | |
| P13716_C223 | ALAD Delta-aminolevulinic | 3518 |
| | acid dehydratase | |
| P12270_C1127 | TPR Nucleoprotein TPR | 3519 |
| Q92973_C205 | TNPO1 Transportin-1 | 3520 |
| P24928_C981 | POLR2A DNA-directed RNA | 3521 |
| | polymerase II subunit RPB1 | |
| P52597_C22 | HNRNPF Heterogeneous | 3522 |
| | nuclear ribonucleoprotein F | |
| Q8TD30_C376 | GPT2 Alanine | 3523 |
| | aminotransferase 2 | |
| Q9Y305_C128 | ACOT9 Acyl-coenzyme A | 3524 |
| | thioesterase 9, mitochondrial | |
| Q8N163_C754 | KIAA1967 DBIRD complex | 3525 |
| | subunit KIAA1967 | |
| Q15149_C530 | PLEC Plectin | 3526 |
| Q15149_C545 | PLEC Plectin | 3527 |
| Q9BX68_C75 | HINT2 Histidine triad | 3528 |
| | nucleotide-binding protein 2, | |
| | mitochondrial | |
| Q9ULW0_C145 | TPX2 Targeting protein for | 3529 |
| | Xklp2 | |
| O14744_C22 | PRMT5 Protein arginine N- | 3530 |
| | methyltransferase 5 | |
| Q5JRX3_C692 | PITRM1 Presequence | 3531 |
| | protease, mitochondrial | |
| P07195_C132 | LDHB L-lactate | 3532 |
| | dehydrogenase B chain | |
| Q96F07_C98 | CYFIP2 Cytoplasmic FMR1- | 3533 |
| | interacting protein 2 | |
| Q9P0K7_C584 | RAI14 Ankycorbin | 3534 |
| P22314_C494 | UBA1 Ubiquitin-like modifier- | 3535 |
| | activating enzyme 1 | |
| P78527_C478 | PRKDC DNA-dependent | 3536 |
| | protein kinase catalytic subunit | |
| P78527_C3187 | PRKDC DNA-dependent | 3537 |
| | protein kinase catalytic subunit | |
| P78527_C3781 | PRKDC DNA-dependent | 3538 |
| | protein kinase catalytic subunit | |
| P42566_C586 | EPS15 Epidermal growth | 3539 |
| | factor receptor substrate 15 | |
| Q86U28_C146 | ISCA2 Iron-sulfur cluster | 3540 |
| | assembly 2 homolog, | |
| | mitochondrial | |
| Q96HE7_C131 | ERO1L ERO1-like protein | 3541 |
| | alpha | |
| Q04724_C526 | TLE1 Transducin-like | 3542 |
| | enhancer protein 1 | |
| Q14789_C2664 | GOLGB1 Golgin subfamily B | 3543 |
| | member 1 | |
| Q9NUQ3_C101 | TXLNG Gamma-taxilin | 3544 |
| Q9ULX6_C128 | AKAP8L A-kinase anchor | 3545 |
| | protein 8-like | |
| Q12905_C271 | ILF2 Interleukin enhancer- | 3546 |
| | binding factor 2 | |
| P23588_C457 | EIF4B Eukaryotic translation | 3547 |
| | initiation factor 4B | |
| P50991_C379 | CCT4 T-complex protein 1 | 3548 |
| | subunit delta | |
| P50991_C450 | CCT4 T-complex protein 1 | 3549 |
| | subunit delta | |
| B0V043_C682 | VARS Valyl-tRNA synthetase | 3550 |
| P22307_C495 | SCP2 Non-specific lipid- | 3551 |
| | transfer protein | |
| Q13472_C244 | TOP3A DNA topoisomerase | 3552 |
| | 3-alpha | |
| Q14315_C1103 | FLNC Filamin-C | 3553 |
| O43747_C539 | AP1G1 AP-1 complex subunit | 3554 |
| | gamma-1 | |
| P23434_C85 | GCSH Glycine cleavage | 3555 |
| | system H protein, | |
| | mitochondrial | |
| Q9H9A6_C23 | LRRC40 Leucine-rich repeat- | 3556 |
| | containing protein 40 | |
| Q8IXH7_C195 | TH1L Negative elongation | 3557 |
| | factor C/D | |
| P22681_C840 | CBL E3 ubiquitin-protein | 3558 |
| | ligase CBL | |
| Q9NVS2_C186 | MRPS18A 28S ribosomal | 3559 |
| | protein S18a, mitochondrial | |
| Q8NF50_C2091 | DOCK8 Dedicator of | 3560 |
| | cytokinesis protein 8 | |
| P09110_C123 | ACAA1 3-ketoacyl-CoA | 3561 |
| | thiolase, peroxisomal | |
| Q8TAF3_C342 | WDR48 WD repeat-containing | 3562 |
| | protein 48 | |
| Q96S55_C347 | WRNIP1 ATPase WRNIP1 | 3563 |
| P45973_C133 | CBX5 Chromobox protein | 3564 |
| | homolog 5 | |
| Q92785_C53 | DPF2 Zinc finger protein ubi- | 3565 |
| | d4 | |
| O43175_C254 | PHGDH D-3- | 3566 |
| | phosphoglycerate | |
| | dehydrogenase | |
| P11388_C405 | TOP2A DNA topoisomerase | 3567 |
| | 2-alpha | |
| Q14699_C211 | RFTN1 Raftlin | 3568 |
| P19367_C823 | HK1 Hexokinase-1 | 3569 |
| Q9BV38_C384 | WDR18 WD repeat-containing | 3570 |
| | protein 18 | |
| Q27J81_C38 | INF2 Inverted formin-2 | 3571 |
| Q27J81_C1093 | INF2 Inverted formin-2 | 3572 |
| Q16740_C86 | CLPP Putative ATP-dependent | 3573 |
| | Clp protease proteolytic | |
| | subunit | |
| P55010_C59 | EIF5 Eukaryotic translation | 3574 |
| | initiation factor 5 | |
| P50213_C331 | IDH3A Isocitrate | 3575 |
| | dehydrogenase | |
| Q9Y692_C113 | GMEB1 Glucocorticoid | 3576 |
| | modulatory element-binding | |
| | protein | |
| Q9C0C9_C341 | UBE2O Ubiquitin-conjugating | 3577 |
| | enzyme E2 O | |
| O15392_C84 | BIRC5 Baculoviral IAP | 3578 |
| | repeat-containing protein 5 | |
| O75533_C1035 | SF3B1 Splicing factor 3B | 3579 |
| | subunit 1 | |
| Q14192_C132 | FHL2 Four and a half LIM | 3580 |
| | domains protein 2 | |
| P33991_C162 | MCM4 DNA replication | 3581 |
| | licensing factor MCM4 | |
| O60684_C208 | KPNA6 Importin subunit | 3582 |
| | alpha-7 | |
| Q92841_C277 | DDX17 Probable ATP- | 3583 |
| | dependent RNA helicase | |
| | DDX17 | |
| P07814_C692 | EPRS Bifunctional | 3584 |
| | glutamate/proline--tRNA | |
| | ligase | |
| P07814_C1076 | EPRS Bifunctional | 3585 |
| | glutamate/proline--tRNA | |
| | ligase | |
| P22102_C733 | GART Trifunctional purine | 3586 |
| | biosynthetic protein adenosine- | |
| | 3 | |
| P32322_C159 | PYCR1 Pyrroline-5- | 3587 |
| | carboxylate reductase 1, | |
| | mitochondrial | |
| O75794_C212 | CDC123 Cell division cycle | 3588 |
| | protein 123 homolog | |
| Q02750_C341 | MAP2K1 Dual specificity | 3589 |
| | mitogen-activated protein | |
| | kinase | |
| Q96RL1_C257 | UIMC1 BRCA1-A complex | 3590 |
| | subunit RAP80 | |
| O15371_C196 | EIF3D Eukaryotic translation | 3591 |
| | initiation factor 3 subunit | |
| Q7L2E3_C346 | DHX30 Putative ATP- | 3592 |
| | dependent RNA helicase | |
| | DHX30 | |
| O00159_C763 | MYO1C Unconventional | 3593 |
| | myosin-Ic | |
| Q8WTW3_C72 | COG1 Conserved oligomeric | 3594 |
| | Golgi complex subunit 1 | |
| Q96KP1_C627 | EXOC2 Exocyst complex | 3595 |
| | component 2 | |
| P62136_C155 | PPP1CA Serine/threonine- | 3596 |
| | protein phosphatase PP1-alpha | |
| | catalytic | |
| Q9UNH7_C348 | SNX6 Sorting nexin-6 | 3597 |
| Q86UY8_C276 | NT5DC3 5-nucleotidase | 3598 |
| | domain-containing protein 3 | |
| P14866_C151 | HNRNPL Heterogeneous | 3599 |
| | nuclear ribonucleoprotein L | |
| P40123_C32 | CAP2 Adenylyl cyclase- | 3600 |
| | associated protein 2 | |
| Q86VQ1_C461 | GLCCII Glucocorticoid- | 3601 |
| | induced transcript 1 protein | |
| Q13451_C394 | FKBP5 Peptidyl-prolyl cis- | 3602 |
| | trans isomerase FKBP5 | |
| Q9P2E9_C1216 | RRBP1 Ribosome-binding | 3603 |
| | protein 1 | |
| P51570_C351 | GALK1 Galactokinase | 3604 |
| Q9NSE4_C155 | IARS2 Isoleucine--tRNA | 3605 |
| | ligase, mitochondrial | |
| Q9BTC0_C976 | DIDO1 Death-inducer | 3606 |
| | obliterator 1 | |
| P07355_C335 | ANXA2 Annexin A2 | 3607 |
| Q5M775_C578 | SPECC1 Cytospin-B | 3608 |
| P17987_C218 | TCP1 T-complex protein 1 | 3609 |
| | subunit alpha | |
| Q99614_C149 | TTC1 Tetratricopeptide repeat | 3610 |
| | protein 1 | |
| O75663_C75 | TIPRL TIP41-like protein | 3611 |
| Q52LW3_C1066 | ARHGAP29 Rho GTPase- | 3612 |
| | activating protein 29 | |
| H3BQZ7_C405 | Uncharacterized protein | 3613 |
| Q9BUL9_C16 | RPP25 Ribonuclease P protein | 3614 |
| | subunit p25 | |
| Q15286_C163 | RAB35 Ras-related protein | 3615 |
| | Rab-35 | |
| P16278_C393 | GLB1 Beta-galactosidase | 3616 |
| Q9HC38_C197 | GLOD4 Glyoxalase domain- | 3617 |
| | containing protein 4 | |
| Q9UM22_C88 | EPDR1 Mammalian | 3618 |
| | ependymin-related protein 1 | |
| Q15056_C85 | EIF4H Eukaryotic translation | 3619 |
| | initiation factor 4H | |
| P23284_C202 | PPIB Peptidyl-prolyl cis-trans | 3620 |
| | isomerase B | |
| P46777_C100 | RPL5 60S ribosomal protein | 3621 |
| | L5 | |
| Q68CZ2_C928 | TNS3 Tensin-3 | 3622 |
| Q68CZ2_C1251 | TNS3 Tensin-3 | 3623 |
| P18031_C215 | PTPN1 Tyrosine-protein | 3624 |
| | phosphatase non-receptor type | |
| | 1 | |
| O95456_C106 | PSMG1 Proteasome assembly | 3625 |
| | chaperone 1 | |
| O95453_C543 | PARN Poly(A)-specific | 3626 |
| | ribonuclease PARN | |
| Q5VTL8_C113 | PRPF38B Pre-mRNA-splicing | 3627 |
| | factor 38B | |
| P31948_C370 | STIP1 Stress-induced- | 3628 |
| | phosphoprotein 1 | |
| O95071_C1476 | UBR5 E3 ubiquitin-protein | 3629 |
| | ligase UBR5 | |
| Q9NQW6_C260 | ANLN Actin-binding protein | 3630 |
| | anillin | |
| Q9NQW6_C437 | ANLN Actin-binding protein | 3631 |
| | anillin | |
| Q9NQW6_C512 | ANLN Actin-binding protein | 3632 |
| | anillin | |
| P51649_C502 | ALDH5A1 Succinate- | 3633 |
| | semialdehyde dehydrogenase, | |
| | mitochondria | |
| P11802_C78 | CDK4 Cyclin-dependent | 3634 |
| | kinase 4 | |
| P49915_C631 | GMPS GMP synthase | 3635 |
| Q9UKN8_C116 | GTF3C4 General transcription | 3636 |
| | factor 3C polypeptide 4 | |
| O60504_C482 | SORBS3 Vinexin | 3637 |
| Q9Y490_C956 | TLN1 Talin-1 | 3638 |
| P23381_C309 | WARS Tryptophan--tRNA | 3639 |
| | ligase, cytoplasmic | |
| P27708_C1696 | CAD CAD protein | 3640 |
| O75607_C79 | NPM3 Nucleoplasmin-3 | 3641 |
| O75131_C506 | CPNE3 Copine-3 | 3642 |
| P38646_C66 | HSPA9 Stress-70 protein, | 3643 |
| | mitochondrial | |
| P49588_C671 | AARS Alanine--tRNA ligase, | 3644 |
| | cytoplasmic | |
| P07602_C33 | PSAP Proactivator polypeptide | 3645 |
| P28065_C63 | PSMB9 Proteasome subunit | 3646 |
| | beta type-9 | |
| P15498_C794 | VAV1 Proto-oncogene vav | 3647 |
| P08559_C273 | PDHA1 Pyruvate | 3648 |
| | dehydrogenase E1 component | |
| | subunit alpha, | |
| Q8WZ82_C129 | OVCA2 Ovarian cancer- | 3649 |
| | associated gene 2 protein | |
| P46939_C1627 | UTRN Utrophin | 3650 |
| Q14166_C361 | TTLL12 Tubulin--tyrosine | 3651 |
| | ligase-like protein 12 | |
| O43314_C488 | PPIP5K2 Inositol | 3652 |
| | hexakisphosphate and | |
| | diphosphoinositol- | |
| | pentakisphosphate kinase 2 | |
| P60981_C147 | DSTN Destrin | 3653 |
| A6NHR9_C444 | SMCHD1 Structural | 3654 |
| | maintenance of chromosomes | |
| | flexible hinge domain | |
| | containing 1 | |
| Q9Y2X0_C539 | MED16 Mediator of RNA | 3655 |
| | polymerase II transcription | |
| | subunit | |
| Q5VYS8_C70 | ZCCHC6 Terminal | 3656 |
| | uridylyltransferase 7 | |
| P24298_C450 | GPT Alanine aminotransferase | 3657 |
| | 1 | |
| P42331_C448 | ARHGAP25 Rho GTPase- | 3658 |
| | activating protein 25 | |
| Q14573_C1638 | ITPR3 Inositol 1,4,5- | 3659 |
| | trisphosphate receptor type 3 | |
| O75717_C716 | WDHD1 WD repeat and | 3660 |
| | HMG-box DNA-binding | |
| | protein 1 | |
| Q9NYV4_C723 | CDK12 Cyclin-dependent | 3661 |
| | kinase 12 | |
| P62487_C38 | POLR2G DNA-directed RNA | 3662 |
| | polymerase II subunit RPB7 | |
| Q96I99_C369 | SUCLG2 Succinyl-CoA ligase | 3663 |
| O00629_C57 | KPNA4 Importin subunit | 3664 |
| | alpha-4 | |
| Q9BW85_C275 | CCDC94 Coiled-coil domain- | 3665 |
| | containing protein 94 | |
| Q9NUQ6_C367 | SPATS2L SPATS2-like | 3666 |
| | protein | |
| Q5VWQ0_C282 | RSBN1 Round spermatid basic | 3667 |
| | protein 1 | |
| Q92835_C1050 | INPP5D Phosphatidylinositol | 3668 |
| | 3,4,5-trisphosphate 5- | |
| | phosphatase 1 | |
| Q9Y6E0_C415 | STK24 Serine/threonine- | 3669 |
| | protein kinase 24 | |
| P49458_C39 | SRP9 Signal recognition | 3670 |
| | particle 9 kDa protein | |
| O43795_C613 | MYO1B Unconventional | 3671 |
| | myosin-Ib | |
| Q9UPN3_C4429 | MACF1 Microtubule-actin | 3672 |
| | cross-linking factor 1, isoforms | |
| P16152_C122 | CBR1 Carbonyl reductase | 3673 |
| Q9BWU0_C224 | SLC4A1AP Kanadaptin | 3674 |
| O75369_C2115 | FLNB Filamin-B | 3675 |
| P48147_C255 | PREP Prolyl endopeptidase | 3676 |
| Q6PL24_C161 | TMED8 Protein TMED8 | 3677 |
| P21980_C10 | TGM2 Protein-glutamine | 3678 |
| | gamma-glutamyltransferase 2 | |
| Q16270_C59 | IGFBP7 Insulin-like growth | 3679 |
| | factor-binding protein 7 | |
| P25205_C446 | MCM3 DNA replication | 3680 |
| | licensing factor MCM3 | |
| Q16181_C280 | SEPT7 Septin-7 | 3681 |
| P83369_C52 | LSM11 U7 snRNA-associated | 3682 |
| | Sm-like protein LSm11 | |
| Q01813_C123 | PFKP 6-phosphofructokinase | 3683 |
| | type C | |
| P49792_C3071 | RANBP2 E3 SUMO-protein | 3684 |
| | ligase RanBP2 | |
| O76031_C586 | CLPX ATP-dependent Clp | 3685 |
| | protease ATP-binding subunit | |
| | clp | |
| Q5T2R2_C315 | PDSS1 Decaprenyl- | 3686 |
| | diphosphate synthase subunit 1 | |
| Q96T76_C356 | MMS19 MMS19 nucleotide | 3687 |
| | excision repair protein | |
| | homolog | |
| Q12996_C646 | CSTF3 Cleavage stimulation | 3688 |
| | factor subunit 3 | |
| P35250_C255 | RFC2 Replication factor C | 3689 |
| | subunit 2 | |
| Q5VVQ6_C97 | YOD1 Ubiquitin thioesterase | 3690 |
| | OTU1 | |
| Q9BXP5_C640 | SRRT Serrate RNA effector | 3691 |
| | molecule homolog | |
| P62701_C181 | RPS4X 40S ribosomal protein | 3692 |
| | S4, X isoform | |
| Q09028_C138 | RBBP4 Histone-binding | 3693 |
| | protein RBBP4 | |
| Q8WXH0_C39 | SYNE2 Nesprin-2 | 3694 |
| P62633_C111 | CNBP Cellular nucleic acid- | 3695 |
| | binding protein | |
| Q8N4X5_C292 | AFAP1L2 Actin filament- | 3696 |
| | associated protein 1-like 2 | |
| Q9HAS0_C182 | C17orf75 Protein Njmu-R1 | 3697 |
| O94806_C29 | PRKD3 Serine/threonine- | 3698 |
| | protein kinase D3 | |
| Q7Z6Z7_C2181 | HUWE1 E3 ubiquitin-protein | 3699 |
| | ligase HUWE1 | |
| P14598_C111 | NCF1 Neutrophil cytosol | 3700 |
| | factor 1 | |
| P23193_C212 | TCEA1 Transcription | 3701 |
| | elongation factor A protein 1 | |
| P60953_C105 | CDC42 Cell division control | 3702 |
| | protein 42 homolog | |
| Q03426_C339 | MVK Mevalonate kinase | 3703 |
| Q9H6S3_C358 | EPS8L2 Epidermal growth | 3704 |
| | factor receptor kinase substrate | |
| P30566_C113 | ADSL Adenylosuccinate lyase | 3705 |
| P51610_C352 | HCFC1 Host cell factor 1 | 3706 |
| Q92616_C1275 | GCN1L1 Translational | 3707 |
| | activator GCN1 | |
| Q92616_C2469 | GCN1L1 Translational | 3708 |
| | activator GCN1 | |
| Q92614_C1080 | MYO18A Unconventional | 3709 |
| | myosin-XVIIIa | |
| Q9BZK7_C434 | TBL1XR1 F-box-like/WD | 3710 |
| | repeat-containing protein | |
| | TBL1XR1 | |
| P53618_C623 | COPB1 Coatomer subunit beta | 3711 |
| Q8TEX9_C42 | IPO4 Importin-4 | 3712 |
| P49327_C1548 | FASN Fatty acid synthase | 3713 |
| Q16875_C155 | PFKFB3 6-phosphofructo-2- | 3714 |
| | kinase/fructose-2,6- | |
| | bisphosphatase | |
| P20839_C331 | IMPDH1 Inosine-5- | 3715 |
| | monophosphate dehydrogenase | |
| | 1 | |
| P40926_C138 | MDH2 Malate dehydrogenase, | 3716 |
| | mitochondrial | |
| P80297_C50 | MT1X Metallothionein-1X | 3717 |
| Q12769_C1166 | NUP160 Nuclear pore | 3718 |
| | complex protein Nup160 | |
| P48059_C100 | LIMS1 LIM and senescent cell | 3719 |
| | antigen-like-containing domain | |
| Q8TBC4_C82 | UBA3 NEDD8-activating | 3720 |
| | enzyme E1 catalytic subunit | |
| P26639_C107 | TARS Threonine--tRNA | 3721 |
| | ligase, cytoplasmic | |
| P13804_C53 | ETFA Electron transfer | 3722 |
| | flavoprotein subunit alpha, | |
| | mitochondrial | |
| A6NE09_C148 | RPSAP58 Protein RPSAP58 | 3723 |
| Q12888_C513 | TP53BP1 Tumor suppressor | 3724 |
| | p53-binding protein 1 | |
| Q02252_C368 | ALDH6A1 Methylmalonate- | 3725 |
| | semialdehyde dehydrogenase | |
| P29144_C967 | TPP2 Tripeptidyl-peptidase 2 | 3726 |
| Q9H1B7_C63 | IRF2BPL Interferon regulatory | 3727 |
| | factor 2-binding protein-like | |
| Q99836_C280 | MYD88 Myeloid | 3728 |
| | differentiation primary | |
| | response protein M | |
| P43487_C158 | RANBP1 Ran-specific | 3729 |
| | GTPase-activating protein | |
| Q6KC79_C304 | NIPBL Nipped-B-like protein | 3730 |
| Q9BTE3_C200 | MCMBP Mini-chromosome | 3731 |
| | maintenance complex-binding | |
| | protein | |
| P42574_C170 | CASP3 Caspase-3 | 3732 |
| Q9NPI6_C39 | DCP1A mRNA-decapping | 3733 |
| | enzyme 1A | |
| O95425_C1101 | SVIL Supervillin | 3734 |
| Q9NS86_C169 | LANCL2 LanC-like protein 2 | 3735 |
| Q8NC60_C393 | NOA1 Nitric oxide-associated | 3736 |
| | protein 1 | |
| O14773_C537 | TPP1 Tripeptidyl-peptidase 1 | 3737 |
| P61088_C87 | UBE2N Ubiquitin-conjugating | 3738 |
| | enzyme E2 N | |
| Q5JRX3_C241 | PITRM1 Presequence | 3739 |
| | protease, mitochondrial | |
| Q5JRX3_C869 | PITRM1 Presequence | 3740 |
| | protease, mitochondrial | |
| Q9BRP1_C100 | PDCD2L Programmed cell | 3741 |
| | death protein 2-like | |
| P61981_C194 | YWHAG 14-3-3 protein gamma | 3742 |
| P01871_C134 | IGHM Ig mu chain C region | 3743 |
| P01871_C451 | IGHM Ig mu chain C region | 3744 |
| Q5T6F2_C104 | UBAP2 Ubiquitin-associated protein 2 | 3745 |
| Q96TA1_C154 | FAM129B Niban-like protein 1 | 3746 |
| P31040_C305 | SDHA Succinate dehydrogenase | 3747 |
| Q96CD2_C7 | PPCDC | 3748 |
| | Phosphopantothenoylcysteine | |
| | decarboxylase | |
| Q9NVU0_C553 | POLR3E DNA-directed RNA | 3749 |
| | polymerase III subunit RPC5 | |
| Q92888_C911 | ARHGEF1 Rho guanine nucleotide | 3750 |
| | exchange factor 1 | |
| Q15020_C537 | SART3 Squamous cell carcinoma | 3751 |
| | antigen recognized by T-cells 3 | |
| Q15022_C46 | SUZ12 Polycomb protein SUZ12 | 3752 |
| Q9UQR0_C559 | SCML2 Sex comb on midleg-like | 3753 |
| | protein 2 | |
| Q14315_C1653 | FLNC Filamin-C | 3754 |
| O94925_C266 | GLS Glutaminase kidney isoform, | 3755 |
| | mitochondrial | |
| Q8NFF5_C561 | FLAD1 FAD synthase | 3756 |
| Q99967_C261 | CITED2 Cbp/p300-interacting | 3757 |
| | transactivator 2 | |
| Q9P287_C141 | BCCIP BRCA2 and CDKN1A- | 3758 |
| | interacting protein | |
| Q7LBC6_C529 | KDM3B Lysine-specific demethylase | 3759 |
| | 3B | |
| Q7LBC6_C569 | KDM3B Lysine-specific demethylase | 3760 |
| | 3B | |
| Q9UGU0_C868 | TCF20 Transcription factor 20 | 3761 |
| Q14683_C1073 | SMC1A Structural maintenance of | 3762 |
| | chromosomes protein 1A | |
| P61513_C39 | RPL37A 60S ribosomal protein L37a | 3763 |
| Q15796_C380 | SMAD2 Mothers against | 3764 |
| | decapentaplegic homolog 2 | |
| Q13547_C100 | HDAC1 Histone deacetylase 1 | 3765 |
| Q9NZT2_C330 | OGFR Opioid growth factor receptor | 3766 |
| P55795_C22 | HNRNPH2 Heterogeneous nuclear | 3767 |
| | ribonucleoprotein H2 | |
| P51812_C229 | RPS6KA3 Ribosomal protein S6 kinase | 3768 |
| | alpha-3 | |
| O15294_C158 | OGT UDP-N-acetylglucosamine-- | 3769 |
| | peptide N-acetylglucosamine (GlcNAc) | |
| | transferase | |
| Q08211_C242 | DHX9 ATP-dependent RNA helicase A | 3770 |
| O43823_C631 | AKAP8 A-kinase anchor protein 8 | 3771 |
| O95819_C883 | MAP4K4 Mitogen-activated protein | 3772 |
| | kinase kinase kinase kinase 4 | |
| Q96AE4_C332 | FUBP1 Far upstream element-binding | 3773 |
| | protein 1 | |
| Q99798_C592 | ACO2 Aconitate hydratase, | 3774 |
| | mitochondrial | |
| Q99797_C518 | M1PEP Mitochondrial intermediate | 3775 |
| | peptidase | |
| O43776_C511 | NARS Asparagine--tRNA ligase, | 3776 |
| | cytoplasmic | |
| P35579_C931 | MYH9 Myosin-9 | 3777 |
| Q9H078_C572 | CLPB Caseinolytic peptidase B protein | 3778 |
| | homolog | |
| Q02962_C52 | PAX2 Paired box protein Pax-2 | 3779 |
| Q9NUG6_C60 | PDRG1 p53 and DNA damage- | 3780 |
| | regulated protein 1 | |
| P22692_C174 | IGFBP4 Insulin-like growth factor- | 3781 |
| | binding protein 4 | |
| Q9BW19_C95 | KIFC1 Kinesin-like protein KIFC1 | 3782 |
| P62879_C294 | GNB2 Guanine nucleotide-binding | 3783 |
| | protein G(I)/G(S)/G(T) | |
| Q9C0C2_C631 | TNKS1BP1 182 kDa tankyrase-1- | 3784 |
| | binding protein | |
| Q9UKG1_C462 | APPL1 DCC-interacting protein 13- | 3785 |
| | alpha | |
| Q12841_C113 | FSTL1 Follistatin-related protein 1 | 3786 |
| O43837_C232 | IDH3B Isocitrate dehydrogenase | 3787 |
| P22102_C291 | GART Trifunctional purine | 3788 |
| | biosynthetic protein adenosine | |
| P22102_C322 | GART Trifunctional purine | 3789 |
| | biosynthetic protein adenosine | |
| P46940_C1534 | IQGAP1 Ras GTPase-activating-like | 3790 |
| | protein IQGAP1 | |
| P11586_C326 | MTHFD1 C-1-tetrahydrofolate | 3791 |
| | synthase, cytoplasmic | |
| P27816_C181 | MAP4 Microtubule-associated protein 4 | 3792 |
| Q9NR46_C252 | SH3GLB2 Endophilin-B2 | 3793 |
| P55060_C387 | CSE1L Exportin-2 | 3794 |
| Q9UQ80_C179 | PA2G4 Proliferation-associated protein | 3795 |
| | 2G4 | |
| Q9NQR4_C170 | NIT2 Omega-amidase NIT2 | 3796 |
| P52306_C85 | RAP1GDS1 Rap1 GTPase-GDP | 3797 |
| | dissociation stimulator 1 | |
| O00571_C298 | DDX3X ATP-dependent RNA helicase | 3798 |
| | DDX3X | |
| Q7L5N1_C283 | COPS6 COP9 signalosome complex | 3799 |
| | subunit 6 | |
| Q01082_C604 | SPTBN1 Spectrin beta chain, non- | 3800 |
| | erythrocytic 1 | |
| O43719_C480 | HTATSF1 HIV Tat-specific factor 1 | 3801 |
| Q9NPB8_C640 | GPCPD1 Glycerophosphocholine | 3802 |
| | phosphodiesterase GPCPD1 | |
| P34932_C417 | HSPA4 Heat shock 70 kDa protein 4 | 3803 |
| P34932_C779 | HSPA4 Heat shock 70 kDa protein 4 | 3804 |
| P21291_C58 | CSRP1 Cysteine and glycine-rich | 3805 |
| | protein 1 | |
| Q9UHR5_C172 | SAP30BP SAP30-binding protein | 3806 |
| P31937_C211 | HIBADH 3-hydroxyisobutyrate | 3807 |
| | dehydrogenase, mitochondrial | |
| P04843_C477 | RPN1 Dolichyl- | 3808 |
| | diphosphooligosaccharide--protein | |
| | glycosyltransferase subunit 1 | |
| Q14980_C658 | NUMA1 Nuclear mitotic apparatus | 3809 |
| | protein 1 | |
| Q9BYV8_C171 | CEP41 Centrosomal protein of 41 kDa | 3810 |
| Q8IUC6_C485 | TICAM1 TIR domain-containing | 3811 |
| | adapter molecule 1 | |
| Q08378_C769 | GOLGA3 Golgin subfamily A member | 3812 |
| | 3 | |
| Q12912_C72 | LRMP Lymphoid-restricted membrane | 3813 |
| | protein | |
| Q9UBD5_C711 | ORC3 Origin recognition complex | 3814 |
| | subunit 3 | |
| O00743_C262 | PPP6C Serine/threonine-protein | 3815 |
| | phosphatase 6 catalytic subunit | |
| Q6P1J9_C145 | CDC73 Parafibromin | 3816 |
| Q1KMD3_C518 | HNRNPUL2 Heterogeneous nuclear | 3817 |
| | ribonucleoprotein U-like protein | |
| Q9P2J5_C70 | LARS Leucine--tRNA ligase, | 3818 |
| | cytoplasmic | |
| Q9P2J5_C83 | LARS Leucine--tRNA ligase, | 3819 |
| | cytoplasmic | |
| P14678_C19 | SNRPB Small nuclear | 3820 |
| | ribonucleoprotein-associated protein | |
| Q9H845_C613 | ACAD9 Acyl-CoA dehydrogenase | 3821 |
| | family member 9, mitochondrial | |
| Q6KB66_C49 | KRT80 Keratin, type II cytoskeletal 80 | 3822 |
| O43143_C190 | DHX15 Putative pre-mRNA-splicing | 3823 |
| | factor ATP-dependent RN | |
| P67775_C20 | PPP2CA Serine/threonine-protein | 3824 |
| | phosphatase 2A catalytic | |
| P04062_C287 | GBA Glucosylceramidase | 3825 |
| Q05086_C108 | UBE3A Ubiquitin-protein ligase E3A | 3826 |
| Q9NPA8_C40 | ENY2 Enhancer of yellow 2 | 3827 |
| | transcription factor homolog | |
| Q9NWZ3_C13 | IRAK4 Interleukin-1 receptor- | 3828 |
| | associated kinase 4 | |
| Q03001_C2128 | DST Dystonin | 3829 |
| P48634_C437 | PRRC2A Protein PRRC2A | 3830 |
| P55196_C1206 | MLLT4 Afadin | 3831 |
| P55196_C1684 | MLLT4 Afadin | 3832 |
| Q9P0W2_C177 | HMG20B SWI/SNF-related matrix- | 3833 |
| | associated actin-dependent | |
| Q8WWI1_C261 | LMO7 LIM domain only protein 7 | 3834 |
| P49903_C71 | SEPHS1 Selenide, water dikinase 1 | 3835 |
| P52435_C31 | POLR2J DNA-directed RNA | 3836 |
| | polymerase II subunit RPB11-a | |
| O43865_C292 | AHCYL1 Putative | 3837 |
| | adenosylhomocysteinase 2 | |
| Q92900_C188 | UPF1 Regulator of nonsense transcripts | 3838 |
| | 1 | |
| Q92871_C57 | PMM1 Phosphomannomutase 1 | 3839 |
| Q15942_C433 | ZYX Zyxin | 3840 |
| Q6UB35_C61 | MTHFD1L Monofunctional C1- | 3841 |
| | tetrahydrofolate synthase, | |
| | mitochondrial | |
| Q9BZF9_C980 | UACA Uveal autoantigen with coiled- | 3842 |
| | coil domains and ankyrin repeats | |
| Q9NYQ6_C1840 | CELSR1 Cadherin EGF LAG seven- | 3843 |
| | pass G-type receptor 1 | |
| O14936_C633 | CASK Peripheral plasma membrane | 3844 |
| | protein CASK | |
| Q96C36_C95 | PYCR2 Pyrroline-5-carboxylate | 3845 |
| | reductase 2 | |
| Q2T9J0_C240 | TYSND1 Peroxisomal leader peptide- | 3846 |
| | processing protease | |
| O14980_C119 | XPO1 Exportin-1 | 3847 |
| P31948_C471 | STIP1 Stress-induced-phosphoprotein 1 | 3848 |
| Q9Y2L1_C194 | DIS3 Exosome complex exonuclease | 3849 |
| | RRP44 | |
| Q96EB1_C412 | ELP4 Elongator complex protein 4 | 3850 |
| P23921_C254 | RRM1 Ribonucleoside-diphosphate | 3851 |
| | reductase large subunit | |
| Q12802_C1232 | AKAP13 A-kinase anchor protein 13 | 3852 |
| Q13404_C144 | UBE2V1 Ubiquitin-conjugating | 3853 |
| | enzyme E2 variant 1 | |
| Q8TDX7_C224 | NEK7 Serine/threonine-protein kinase | 3854 |
| | Nek7 | |
| P27708_C2092 | CAD CAD protein | 3855 |
| Q9Y4B6_C1227 | VPRBP Protein VPRBP | 3856 |
| P23497_C96 | SP100 Nuclear autoantigen Sp-100 | 3857 |
| O75592_C2163 | MYCBP2 Probable E3 ubiquitin- | 3858 |
| | protein ligase MYCBP2 | |
| Q12962_C174 | TAF10 Transcription initiation factor | 3859 |
| | TFIID subunit 10 | |
| Q99700_C556 | ATXN2 Ataxin-2 | 3860 |
| Q9Y5L0_C912 | TNPO3 Transportin-3 | 3861 |
| A1KXE4_C63 | FAM168B Myelin-associated neurite- | 3862 |
| | outgrowth inhibitor | |
| Q13671_C284 | RIN1 Ras and Rab interactor 1 | 3863 |
| P62913_C150 | RPL11 60S ribosomal protein L11 | 3864 |
| Q9H832_C100 | UBE2Z Ubiquitin-conjugating enzyme | 3865 |
| | E2 Z | |
| Q00796_C301 | SORD Sorbitol dehydrogenase | 3866 |
| P00491_C206 | PNP Purine nucleoside phosphorylase | 3867 |
| Q9Y5U2_C99 | TSSC4 Protein TSSC4 | 3868 |
| O96017_C539 | CHEK2 Serine/threonine-protein kinase | 3869 |
| | Chk2 | |
| O96019_C152 | ACTL6A Actin-like protein 6A | 3870 |
| P52701_C694 | MSH6 DNA mismatch repair protein | 3871 |
| | Msh6 | |
| P26440_C378 | IVD Isovaleryl-CoA dehydrogenase, | 3872 |
| | mitochondrial | |
| Q16543_C308 | CDC37 Hsp90 co-chaperone Cdc37 | 3873 |
| Q9NUW8_C135 | TDP1 Tyrosyl-DNA phosphodiesterase | 3874 |
| | 1 | |
| Q14139_C79 | UBE4A Ubiquitin conjugation factor | 3875 |
| | E4 A | |
| Q96FV9_C49 | THOC1 THO complex subunit 1 | 3876 |
| P33992_C397 | MCM5 DNA replication licensing | 3877 |
| | factor MCM5 | |
| Q15417_C273 | CNN3 Calponin-3 | 3878 |
| P41567_C69 | EIF1 Eukaryotic translation initiation | 3879 |
| | factor 1 | |
| P30626_C162 | SRI Sorcin | 3880 |
| A6NHR9_C1433 | SMCHD1 Structural maintenance of | 3881 |
| | chromosomes flexible hinge domain | |
| | containing 1 | |
| O00410_C348 | IPO5 Importin-5 | 3882 |
| O00410_C560 | IPO5 Importin-5 | 3883 |
| O60563_C630 | CCNT1 Cyclin-T1 | 3884 |
| P35580_C678 | MYH10 Myosin-10 | 3885 |
| P60228_C345 | EIF3E Eukaryotic translation initiation | 3886 |
| | factor 3 subunit | |
| P49116_C387 | NR2C2 Nuclear receptor subfamily 2 | 3887 |
| | group C member 2 | |
| P42330_C242 | AKR1C3 Aldo-keto reductase family 1 | 3888 |
| | member C3 | |
| O60271_C1155 | SPAG9 C-Jun-amino-terminal kinase- | 3889 |
| | interacting protein 4 | |
| Q96FA3_C61 | PELI1 E3 ubiquitin-protein ligase | 3890 |
| | pellino homolog 1 | |
| Q96IF1_C224 | AJUBA LIM domain-containing | 3891 |
| | protein ajuba | |
| Q9ULK4_C297 | MED23 Mediator of RNA polymerase | 3892 |
| | II transcription subunit | |
| Q9ULK4_C1319 | MED23 Mediator of RNA polymerase | 3893 |
| | II transcription subunit | |
| O00622_C359 | CYR61 Protein CYR61 | 3894 |
| O95985_C217 | TOP3B DNA topoisomerase 3-beta-1 | 3895 |
| P33240_C62 | CSTF2 Cleavage stimulation factor | 3896 |
| | subunit 2 | |
| P52272_C694 | HNRNPM Heterogeneous nuclear | 3897 |
| | ribonucleoprotein M | |
| A0AVT1_C721 | UBA6 Ubiquitin-like modifier- | 3898 |
| | activating enzyme 6 | |
| Q9BZG1_C116 | RAB34 Ras-related protein Rab-34 | 3899 |
| P53384_C25 | NUBP1 Cytosolic Fe—S cluster | 3900 |
| | assembly factor NUBP1 | |
| Q15181_C114 | PPA1 Inorganic pyrophosphatase | 3901 |
| Q9NPF4_C73 | OSGEP Probable tRNA | 3902 |
| | threonylcarbamoyladenosine | |
| | biosynthesis | |
| Q96FF9_C195 | CDCA5 Sororin | 3903 |
| Q8NFC6_C74 | BOD1L1 Biorientation of chromosomes | 3904 |
| | in cell division protein | |
| Q9UPN7_C37 | PPP6R1 Serine/threonine-protein | 3905 |
| | phosphatase 6 regulatory | |
| Q9UPN9_C447 | TRIM33 E3 ubiquitin-protein ligase | 3906 |
| | TRIM33 | |
| J3QR44_C422 | CDK11B Cyclin-dependent kinase 11B | 3907 |
| Q641Q2_C594 | FAM21A WASH complex subunit | 3908 |
| | FAM21A | |
| O75362_C641 | ZNF217 Zinc finger protein 217 | 3909 |
| P78406_C106 | RAE1 mRNA export factor | 3910 |
| Q6P2I3_C215 | FAHD2B Fumarylacetoacetate | 3911 |
| | hydrolase domain-containing protein | |
| P49770_C310 | EIF2B2 Translation initiation factor | 3912 |
| | eIF-2B subunit beta | |
| Q9Y5A9_C482 | YTHDF2 YTH domain family protein 2 | 3913 |
| P25205_C319 | MCM3 DNA replication licensing | 3914 |
| | factor MCM3 | |
| P36959_C186 | GMPR GMP reductase 1 | 3915 |
| O00273_C165 | DFFA DNA fragmentation factor | 3916 |
| | subunit alpha | |
| P49790_C593 | NUP153 Nuclear pore complex protein | 3917 |
| | Nup153 | |
| Q00610_C1528 | CLTC Clathrin heavy chain 1 | 3918 |
| Q9BZ67_C206 | FRMD8 FERM domain-containing | 3919 |
| | protein 8 | |
| Q5VZ89_C850 | DENND4C DENN domain-containing | 3920 |
| | protein 4C | |
| Q8IY18_C881 | SMC5 Structural maintenance of | 3921 |
| | chromosomes protein 5 | |
| Q9BXP5_C490 | SRRT Serrate RNA effector molecule | 3922 |
| | homolog | |
| P29692_C217 | EEF1D Elongation factor 1-delta | 3923 |
| P29692_C247 | EEF1D Elongation factor 1-delta | 3924 |
| P62979_C121 | RPS27A Ubiquitin-40S ribosomal | 3925 |
| | protein S27a | |
| O75083_C507 | WDR1 WD repeat-containing protein 1 | 3926 |
| Q13131_C185 | PRKAA1 5-AMP-activated protein | 3927 |
| | kinase catalytic subunit | |
| P23368_C441 | ME2 NAD-dependent malic enzyme, | 3928 |
| | mitochondrial | |
| P04637_C141 | TP53 Cellular tumor antigen p53 | 3929 |
| Q7Z6Z7_C1074 | HUWE1 E3 ubiquitin-protein ligase | 3930 |
| | HUWE1 | |
| Q16643_C632 | DBN1 Drebrin | 3931 |
| Q9UPQ0_C55 | LIMCH1 LIM and calponin homology | 3932 |
| | domains-containing protein | |
| Q9NS91_C64 | RAD18 E3 ubiquitin-protein ligase | 3933 |
| | RAD18 | |
| Q7Z3D6_C124 | C14orf159 UPF0317 protein | 3934 |
| | C14orf159, mitochondrial | |
| Q53EZ4_C236 | CEP55 Centrosomal protein of 55 kDa | 3935 |
| P62826_C85 | RAN GTP-binding nuclear protein Ran | 3936 |
| P18583_C2070 | SON Protein SON | 3937 |
| Q92616_C932 | GCN1L1 Translational activator GCN1 | 3938 |
| P22087_C99 | FBL rRNA 2-O-methyltransferase | 3939 |
| | fibrillarin | |
| Q96I24_C460 | FUBP3 Far upstream element-binding | 3940 |
| | protein 3 | |
| Q14657_C113 | LAGE3 L antigen family member 3 | 3941 |
| P49327_C2091 | FASN Fatty acid synthase | 3942 |
| P49327_C2468 | FASN Fatty acid synthase | 3943 |
| Q9UDR5_C534 | AASS Alpha-aminoadipic | 3944 |
| | semialdehyde synthase, mitochondial | |
| Q15746_C83 | MYLK Myosin light chain kinase, | 3945 |
| | smooth muscle | |
| O14929_C120 | HAT1 Histone acetyltransferase type B | 3946 |
| | catalytic subunit | |
| Q9NS87_C862 | KIF15 Kinesin-like protein KIF15 | 3947 |
| Q8TBC4_C139 | UBA3 NEDD8-activating enzyme E1 | 3948 |
| | catalytic subunit | |
| Q13596_C318 | SNX1 Sorting nexin-1 | 3949 |
| P55160_C780 | NCKAP1L Nck-associated protein 1- | 3950 |
| | like | |
| P31150_C202 | GDI1 Rab GDP dissociation inhibitor | 3951 |
| | alpha | |
| Q96BN8_C47 | FAM105B Protein FAM105B | 3952 |
| Q12888_C1703 | TP53BP1 Tumor suppressor p53- | 3953 |
| | binding protein 1 | |
| Q9NRY4_C814 | ARHGAP35 Rho GTPase-activating | 3954 |
| | protein 35 | |
| Q9BV68_C15 | RNF126 RING finger protein 126 | 3955 |
| Q92974_C715 | ARHGEF2 Rho guanine nucleotide | 3956 |
| | exchange factor 2 | |
| Q9HAU4_C716 | SMURF2 E3 ubiquitin-protein ligase | 3957 |
| | SMURF2 | |
| P24928_C1470 | POLR2A DNA-directed RNA | 3958 |
| | polymerase II subunit RPB1 | |
| P07858_C108 | CTSB Cathepsin B | 3959 |
| O75190_C275 | DNAJB6 DnaJ homolog subfamily B | 3960 |
| | member 6 | |
| Q9Y6X8_C11 | ZHX2 Zinc fingers and homeoboxes | 3961 |
| | protein 2 | |
| P62263_C85 | RPS14 40S ribosomal protein S14 | 3962 |
| P31146_C195 | CORO1A Coronin-1A | 3963 |
| Q9H2M9_C1336 | RAB3GAP2 Rab3 GTPase-activating | 3964 |
| | protein non-catalytic subunit | |
| O60825_C105 | PFKFB2 6-phosphofructo-2- | 3965 |
| | kinase/fructose-2,6-bisphosphatase 2 | |
| Q9NTJ3_C271 | SMC4 Structural maintenance of | 3966 |
| | chromosomes protein 4 | |
| Q00577_C292 | PURA Transcriptional activator protein | 3967 |
| | Pur-alpha | |
| Q5VT52_C903 | RPRD2 Regulation of nuclear pre- | 3968 |
| | mRNA domain-containing protein | |
| Q9UKV3_C733 | ACIN1 Apoptotic chromatin | 3969 |
| | condensation inducer in the nucleus | |
| Q92552_C49 | MRPS27 28S ribosomal protein S27, | 3970 |
| | mitochondrial | |
| Q96MG7_C283 | NDNL2 Melanoma-associated antigen | 3971 |
| | G1 | |
| Q7L5D6_C205 | GET4 Golgi to ER traffic protein 4 | 3972 |
| | homolog | |
| Q8N556_C471 | AFAP1 Actin filament-associated | 3973 |
| | protein 1 | |
| Q8N556_C506 | AFAP1 Actin filament-associated | 3974 |
| | protein 1 | |
| P62987_C115 | UBA52 Ubiquitin-60S ribosomal | 3975 |
| | protein L40 | |
| Q96HE7_C35 | ERO1L ERO1-like protein alpha | 3976 |
| Q6IBS0_C275 | TWF2 Twinfilin-2 | 3977 |
| Q04724_C89 | TLE1 Transducin-like enhancer protein | 3978 |
| | 1 | |
| Q9H3U1_C650 | UNC45A Protein unc-45 homolog A | 3979 |
| O43374_C797 | RASA4 Ras GTPase-activating protein | 3980 |
| | 4 | |
| Q9NUQ8_C628 | ABCF3 ATP-binding cassette sub- | 3981 |
| | family F member 3 | |
| Q9UJF2_C443 | RASAL2 Ras GTPase-activating | 3982 |
| | protein nGAP | |
| P61160_C11 | ACTR2 Actin-related protein 2 | 3983 |
| Q7Z6M1_C115 | RABEPK Rab9 effector protein with | 3984 |
| | kelch motifs | |
| Q13418_C422 | ILK Integrin-linked protein kinase | 3985 |
| P30153_C317 | PPP2R1A Serine/threonine-protein | 3986 |
| | phosphatase 2A 65 kDa regulatory | |
| | subunit A alpha isoform | |
| P62273_C39 | RPS29 40S ribosomal protein S29 | 3987 |
| P61218_C76 | POLR2F DNA-directed RNA | 3988 |
| | polymerases I, II, and III subunit | |
| P58546_C83 | MTPN Myotrophin | 3989 |
| Q92888_C815 | ARHGEF1 Rho guanine nucleotide | 3990 |
| | exchange factor 1 | |
| Q9BZX2_C241 | UCK2 Uridine-cytidine kinase 2 | 3991 |
| O43815_C316 | STRN Striatin | 3992 |
| O94925_C463 | GLS Glutaminase kidney isoform, | 3993 |
| | mitochondrial | |
| P42224_C155 | STAT1 Signal transducer and activator | 3994 |
| | of transcription 1 | |
| P30837_C169 | ALDH1B1 Aldehyde dehydrogenase X, | 3995 |
| | mitochondrial | |
| P62140_C139 | PPP1CB Serine/threonine-protein | 3996 |
| | phosphatase PP1-beta catalytic | |
| Q6XQN6_C533 | NAPRT1 Nicotinate | 3997 |
| | phosphoribosyltransferase | |
| Q96DM3_C333 | C18orf8 Uncharacterized protein | 3998 |
| | C18orf8 | |
| P33764_C53 | S100A3 Protein S100-A3 | 3999 |
| Q96F86_C91 | EDC3 Enhancer of mRNA-decapping | 4000 |
| | protein 3 | |
| O00221_C187 | NFKBIE NF-kappa-B inhibitor epsilon | 4001 |
| Q9ULC4_C144 | MCTS1 Malignant T-cell-amplified | 4002 |
| | sequence 1 | |
| O60841_C635 | EIF5B Eukaryotic translation initiation | 4003 |
| | factor 5B | |
| Q5TFE4_C179 | NT5DC1 5-nucleotidase domain- | 4004 |
| | containing protein 1 | |
| Q9UNM6_C114 | PSMD13 26S proteasome non-ATPase | 4005 |
| | regulatory subunit 13 | |
| O43847_C1109 | NRD1 Nardilysin | 4006 |
| Q9HAV4_C1157 | XPO5 Exportin-5 | 4007 |
| O43172_C441 | PRPF4 U4/U6 small nuclear | 4008 |
| | ribonucleoprotein Prp4 | |
| Q8IXW5_C105 | RPAP2 Putative RNA polymerase II | 4009 |
| | subunit B1 CTD phosphatase | |
| Q6QNY0_C180 | BLOC1S3 Biogenesis of lysosome- | 4010 |
| | related organelles complex | |
| Q14697_C423 | GANAB Neutral alpha-glucosidase AB | 4011 |
| Q27J81_C394 | INF2 Inverted formin-2 | 4012 |
| P60900_C137 | PSMA6 Proteasome subunit alpha type- | 4013 |
| | 6 | |
| P55786_C66 | NPEPPS Puromycin-sensitive | 4014 |
| | aminopeptidase | |
| P62879_C317 | GNB2 Guanine nucleotide-binding | 4015 |
| | protein G(I)/G(S)/G(T) | |
| Q9Y692_C274 | GMEB1 Glucocorticoid modulatory | 4016 |
| | element-binding protein | |
| O00567_C52 | NOP56 Nucleolar protein 56 | 4017 |
| Q9C0C2_C1296 | TNKS1BP1 182 kDa tankyrase-1- | 4018 |
| | binding protein | |
| Q15008_C58 | PSMD6 26S proteasome non-ATPase | 4019 |
| | regulatory subunit 6 | |
| O75533_C677 | SF3B1 Splicing factor 3B subunit 1 | 4020 |
| E7EVK2_C23 | MTG1 Mitochondrial GTPase 1 | 4021 |
| O95336_C78 | PGLS 6-phosphogluconolactonase | 4022 |
| Q9C0B1_C326 | FTO Alpha-ketoglutarate-dependent | 4023 |
| | dioxygenase FTO | |
| Q8TEU7_C1368 | RAPGEF6 Rap guanine nucleotide | 4024 |
| | exchange factor 6 | |
| P35568_C923 | IRS1 Insulin receptor substrate 1 | 4025 |
| P04049_C637 | RAF1 RAF proto-oncogene | 4026 |
| | serine/threonine-protein kinase | |
| P60660_C138 | MYL6 Myosin light polypeptide 6 | 4027 |
| Q8NDH3_C189 | NPEPL1 Probable aminopeptidase | 4028 |
| | NPEPL1 | |
| P25786_C182 | PSMA1 Proteasome subunit alpha type- | 4029 |
| | 1 | |
| Q7L591_C385 | DOK3 Docking protein 3 | 4030 |
| Q9UJM3_C146 | ERRFI1 ERBB receptor feedback | 4031 |
| | inhibitor 1 | |
| O00159_C679 | MYO1C Unconventional myosin-Ic | 4032 |
| P55060_C61 | CSE1L Exportin-2 | 4033 |
| P14868_C130 | DARS Aspartate--tRNA ligase, | 4034 |
| | cytoplasmic | |
| P14868_C259 | DARS Aspartate--tRNA ligase, | 4035 |
| | cytoplasmic | |
| P45954_C261 | ACADSB Short/branched chain | 4036 |
| | specific acyl-CoA dehydrogenase | |
| Q01664_C29 | TFAP4 Transcription factor AP-4 | 4037 |
| P08138_C381 | NGFR Tumor necrosis factor receptor | 4038 |
| | superfamily member | |
| O60671_C239 | RAD1 Cell cycle checkpoint protein | 4039 |
| | RAD1 | |
| P46379_C856 | BAG6 Large proline-rich protein BAG6 | 4040 |
| P26022_C103 | PTX3 Pentraxin-related protein PTX3 | 4041 |
| Q9BYX2_C651 | TBC1D2 TBC1 domain family member | 4042 |
| | 2A | |
| P23610_C76 | F8A3 Factor VIII intron 22 protein | 4043 |
| Q9NZE8_C119 | MRPL35 39S ribosomal protein L35, | 4044 |
| | mitochondrial | |
| Q9UJZ1_C167 | STOML2 Stomatin-like protein 2 | 4045 |
| P31937_C251 | HIBADH 3-hydroxyisobutyrate | 4046 |
| | dehydrogenase, mitochondrial | |
| Q9NR56_C34 | MBNL1 Muscleblind-like protein 1 | 4047 |
| Q9P0V9_C100 | SEPT10 Septin-10 | 4048 |
| Q96AT9_C23 | RPE Ribulose-phosphate 3-epimerase | 4049 |
| Q08378_C1431 | GOLGA3 Golgin subfamily A member | 4050 |
| | 3 | |
| Q9Y4X5_C161 | ARIH1 E3 ubiquitin-protein ligase | 4051 |
| | ARIH1 | |
| P09417_C161 | QDPR Dihydropteridine reductase | 4052 |
| Q7L014_C377 | DDX46 Probable ATP-dependent RNA | 4053 |
| | helicase DDX46 | |
| Q7L014_C501 | DDX46 Probable ATP-dependent RNA | 4054 |
| | helicase DDX46 | |
| Q92917_C137 | GPKOW G patch domain and KOW | 4055 |
| | motifs-containing protein | |
| P28340_C1058 | POLD1 DNA polymerase delta | 4056 |
| | catalytic subunit | |
| P53350_C212 | PLK1 Serine/threonine-protein kinase | 4057 |
| | PLK1 | |
| Q86VP6_C942 | CAND1 Cullin-associated NEDD8- | 4058 |
| | dissociated protein 1 | |
| P35237_C350 | SERPINB6 Serpin B6 | 4059 |
| P67775_C266 | PPP2CA Serine/threonine-protein | 4060 |
| | phosphatase 2A catalytic | |
| Q14152_C478 | EIF3A Eukaryotic translation initiation | 4061 |
| | factor 3 subunit | |
| P19338_C543 | NCL Nucleolin | 4062 |
| Q16576_C277 | RBBP7 Histone-binding protein | 4063 |
| | RBBP7 | |
| Q5UIP0VC2274 | RIF1 Telomere-associated protein RIF1 | 4064 |
| O60566_C578 | BUB1B Mitotic checkpoint | 4065 |
| | serine/threonine-protein kinase | |
| O60566_C723 | BUB1B Mitotic checkpoint | 4066 |
| | serine/threonine-protein kinase | |
| Q9UBU9_C252 | NXF1 Nuclear RNA export factor 1 | 4067 |
| P41134_C34 | ID1 DNA-binding protein inhibitor ID- | 4068 |
| | 1 | |
| Q03001_C5610 | DST Dystonin | 4069 |
| P49720_C19 | PSMB3 Proteasome subunit beta type-3 | 4070 |
| Q15054_C129 | POLD3 DNA polymerase delta subunit | 4071 |
| | 3 | |
| Q9Y223_C183 | GNE Bifunctional UDP-N- | 4072 |
| | acetylglucosamine 2-epimerase/N | |
| P31323_C373 | PRKAR2B cAMP-dependent protein | 4073 |
| | kinase type II-beta regulatory subunit | |
| | beta | |
| Q08257_C145 | CRYZ Quinone oxidoreductase | 4074 |
| P18031_C344 | PTPN1 Tyrosine-protein phosphatase | 4075 |
| | non-receptor type 1 | |
| P04075_C240 | ALDOA Fructose-bisphosphate | 4076 |
| | aldolase A | |
| Q8WYP5_C1261 | AHCTF1 Protein ELYS | 4077 |
| Q14145_C3I9 | KEAP1 Kelch-like ECH-associated | 4078 |
| | protein 1 | |
| Q14141_C432 | SEPT6 Septin-6 | 4079 |
| P46821_C261 | MAP1B Microtubule-associated protein | 4080 |
| | 1B | |
| O00291_C249 | HIP1 Huntingtin-interacting protein 1 | 4081 |
| Q96A65_C106 | EXOC4 Exocyst complex component 4 | 4082 |
| P34896_C389 | SHMT1 Serine | 4083 |
| | hydroxymethyltransferase, cytosolic | |
| Q6XZF7_C327 | DNMBP Dynamin-binding protein | 4084 |
| O95671_C274 | ASMTL N-acetylserotonin O- | 4085 |
| | methyltransferase-like protein | |
| P09960_C147 | LTA4H Leukotriene A-4 hydrolase | 4086 |
| P09960_C200 | LTA4H Leukotriene A-4 hydrolase | 4087 |
| Q86WB0_C125 | ZC3HC1 Nuclear-interacting partner of | 4088 |
| | ALK | |
| Q96EP5_C63 | DAZAP1 DAZ-associated protein 1 | 4089 |
| Q9Y490_C732 | TLN1 Talin-1 | 4090 |
| Q9Y490_C1506 | TLN1 Talin-1 | 4091 |
| P27708_C2161 | CAD CAD protein | 4092 |
| O95628_C175 | CNOT4 CCR4-NOT transcription | 4093 |
| | complex subunit 4 | |
| Q02535_C16 | ID3 DNA-binding protein inhibitor ID- | 4094 |
| | 3 | |
| P23497_C238 | SP100 Nuclear autoantigen Sp-100 | 4095 |
| P23497_C468 | SP100 Nuclear autoantigen Sp-100 | 4096 |
| Q8IV63_C191 | VRK3 Inactive serine/threonine-protein | 4097 |
| | kinase VRK3 | |
| Q7L5Y1_C307 | ENOSF1 Mitochondrial enolase | 4098 |
| | superfamily member 1 | |
| Q9UBW7_C1331 | ZMYM2 Zinc finger MYM-type | 4099 |
| | protein 2 | |
| O15446_C86 | CD3EAP DNA-directed RNA | 4100 |
| | polymerase I subunit RPA34 | |
| P54136_C312 | RARS Arginine-tRNA ligase, | 4101 |
| | cytoplasmic | |
| P49748_C215 | ACADVL Very long-chain specific | 4102 |
| | acyl-CoA dehydrogenase, | |
| | mitochondrial | |
| Q9BRX2VC175 | PELO Protein pelota homolog | 4103 |
| O95544_C69 | NADK NAD kinase | 4104 |
| Q969W3_C159 | FAM104A Protein FAM104A | 4105 |
| P62917_C195 | RPL8 60S ribosomal protein L8 | 4106 |
| Q00839_C335 | HNRNPU Heterogeneous nuclear | 4107 |
| | ribonucleoprotein U | |
| P00568_C25 | AK1 Adenylate kinase isoenzyme 1 | 4108 |
| D6RAD4_C275 | CDK7 Cyclin-dependent kinase 7 | 4109 |
| P49368_C475 | CCT3 T-complex protein 1 subunit | 4110 |
| | gamma | |
| Q53S33_C59 | BOLA3 BolA-like protein 3 | 4111 |
| Q96A49_C283 | SYAP1 Synapse-associated protein 1 | 4112 |
| Q6ZS81_C1665 | WDFY4 WD repeat- and FYVE | 4113 |
| | domain-containing protein 4 | |
| P60981_C39 | DSTN Destrin | 4114 |
| P51531_C1296 | SMARCA2 Probable global | 4115 |
| | transcription activator SNF2L2 | |
| Q7Z417_C234 | NUFIP2 Nuclear fragile X mental | 4116 |
| | retardation-interacting protein | |
| O00410_C180 | IPO5 Importin-5 | 4117 |
| O00410_C972 | IPO5 Importin-5 | 4118 |
| Q9NTK5_C187 | OLA1 Obg-like ATPase 1 | 4119 |
| P60228_C350 | EIF3E Eukaryotic translation initiation | 4120 |
| | factor 3 subunit | |
| Q96CP2_C132 | FLYWCH2 FLYWCH family member | 4121 |
| | 2 | |
| H0YGG7_C34 | Uncharacterized protein | 4122 |
| Q92538_C1766 | GBF1 Golgi-specific brefeldin A- | 4123 |
| | resistance guanine nucleus | |
| O95081_C39 | AGFG2 Arf-GAP domain and FG | 4124 |
| | repeat-containing protein 2 | |
| Q16531_C977 | DDB1 DNA damage-binding protein 1 | 4125 |
| P28482_C254 | MAPK1 Mitogen-activated protein | 4126 |
| | kinase 1 | |
| P36578_C208 | RPL4 60S ribosomal protein L4 | 4127 |
| Q8NC26_C75 | ZNF114 Zinc finger protein 114 | 4128 |
| Q9BQ69_C199 | MACROD1 O-acetyl-ADP-ribose | 4129 |
| | deacetylase MACROD1 | |
| B2RTY4_C2032 | MYO9A Unconventional myosin-IXa | 4130 |
| Q9NZL9_C17 | MAT2B Methionine | 4131 |
| | adenosyltransferase 2 subunit beta | |
| P30291_C19 | WEE1 Wee1-like protein kinase | 4132 |
| Q53ET0_C675 | CRTC2 CREB-regulated transcription | 4133 |
| | coactivator 2 | |
| P02765_C219 | AHSG Alpha-2-HS-glycoprotein | 4134 |
| Q96T60_C353 | PNKP Bifunctional polynucleotide | 4135 |
| | phosphatase/kinase | |
| P43034_C252 | PAFAH1B1 Platelet-activating factor | 4136 |
| | acetylhydrolase 1B subunit | |
| Q9P215_C39 | POGK Pogo transposable element with | 4137 |
| | KRAB domain | |
| P09001_C338 | MRPL3 39S ribosomal protein L3, | 4138 |
| | mitochondrial | |
| Q8NFC6_C2164 | BOD1L1 Biorientation of chromosomes | 4139 |
| | in cell division protein | |
| Q13126_C136 | MTAP S-methyl-5-thioadenosine | 4140 |
| | phosphorylase | |
| Q9Y6I3_C205 | EPN1 Epsin-1 | 4141 |
| Q9HA47_C236 | UCK1 Uridine-cytidine kinase 1 | 4142 |
| Q96KG9_C88 | SCYL1 N-terminal kinase-like protein | 4143 |
| P49419_C70 | ALDH7A1 Alpha-aminoadipic | 4144 |
| | semialdehyde dehydrogenase | |
| Q96RU3_C511 | FNBP1 Formin-binding protein 1 | 4145 |
| Q96RU2_C733 | USP28 Ubiquitin carboxyl-terminal | 4146 |
| | hydrolase 28 | |
| Q16270_C71 | IGFBP7 Insulin-like growth factor- | 4147 |
| | binding protein 7 | |
| P50395_C302 | GDI2 Rab GDP dissociation inhibitor | 4148 |
| | beta | |
| P50395_C414 | GDI2 Rab GDP dissociation inhibitor | 4149 |
| | beta | |
| P40306_C17 | PSMB10 Proteasome subunit beta type- | 4150 |
| | 10 | |
| Q16181_C204 | SEPT7 Septin-7 | 4151 |
| P60891_C41 | PRPS1 Ribose-phosphate | 4152 |
| | pyrophosphokinase 1 | |
| Q00403_C223 | GTF2B Transcription initiation factor | 4153 |
| | IIB | |
| O95816_C142 | BAG2 BAG family molecular | 4154 |
| | chaperone regulator 2 | |
| Q00610_C926 | CLTC Clathrin heavy chain 1 | 4155 |
| Q00610_C1102 | CLTC Clathrin heavy chain 1 | 4156 |
| Q12996_C176 | CSTF3 Cleavage stimulation factor | 4157 |
| | subunit 3 | |
| Q9BXB5_C33 | OSBPL10 Oxysterol-binding protein- | 4158 |
| | related protein 10 | |
| P46013_C475 | MKI67 Antigen KI-67 | 4159 |
| P46013_C1251 | MKI67 Antigen KI-67 | 4160 |
| O94979_C704 | SEC31A Protein transport protein | 4161 |
| | Sec31A | |
| O75083_C170 | WDR1 WD repeat-containing protein 1 | 4162 |
| O43252_C53 | PAPSS1 Bifunctional 3- | 4163 |
| | phosphoadenosine 5-phosphosulfate | |
| Q13131_C117 | PRKAA1 5-AMP-activated protein | 4164 |
| | kinase catalytic subunit | |
| P62633_C158 | CNBP Cellular nucleic acid-binding | 4165 |
| | protein | |
| P52732_C695 | KIF11 Kinesin-like protein KIF11 | 4166 |
| Q8TD16_C437 | BICD2 Protein bicaudal D homolog 2 | 4167 |
| Q14008_C1768 | CKAP5 Cytoskeleton-associated | 4168 |
| | protein 5 | |
| P60604_C89 | UBE2G2 Ubiquitin-conjugating | 4169 |
| | enzyme E2 G2 | |
| Q9UHY1_C478 | NRBP1 Nuclear receptor-binding | 4170 |
| | protein | |
| Q9BPX3_C317 | NCAPG Condensin complex subunit 3 | 4171 |
| Q9NZJ9_C131 | NUDT4 Diphosphoinositol | 4172 |
| | polyphosphate phosphohydrolase 2 | |
| P21333_C1165 | FLNA Filamin-A | 4173 |
| P13010_C296 | XRCC5 X-ray repair cross- | 4174 |
| | complementing protein 5 | |
| Q9NRX1_C64 | PNO1 RNA-binding protein PNO1 | 4175 |
| Q9NRX2_C129 | MRPL17 39S ribosomal protein L17, | 4176 |
| | mitochondrial | |
| O75414_C163 | NME6 Nucleoside diphosphate kinase 6 | 4177 |
| Q96CU9_C116 | FOXRED1 FAD-dependent | 4178 |
| | oxidoreductase domain-containing | |
| | protein | |
| Q6DN90_C485 | IQSEC1 IQ motif and SEC7 domain- | 4179 |
| | containing protein 1 | |
| P01033_C168 | TIMP1 Metalloproteinase inhibitor 1 | 4180 |
| Q92616_C1161 | GCN1L1 Translational activator GCN1 | 4181 |
| Q53GL7_C50 | PARP10 Poly | 4182 |
| Q9UH65_C260 | SWAP70 Switch-associated protein 70 | 4183 |
| Q9UH65_C261 | SWAP70 Switch-associated protein 70 | 4184 |
| P46060_C338 | RANGAP1 Ran GTPase-activating | 4185 |
| | protein 1 | |
| P68402_C35 | PAFAH1B2 Platelet-activating factor | 4186 |
| | acetylhydrolase IB subunit | |
| P61964_C205 | WDR5 WD repeat-containing protein 5 | 4187 |
| P49327_C223 | FASN Fatty acid synthase | 4188 |
| Q99536_C86 | VAT1 Synaptic vesicle membrane | 4189 |
| | protein VAT-1 homolog | |
| Q8N1G4_C249 | LRRC47 Leucine-rich repeat- | 4190 |
| | containing protein 47 | |
| O14972_C221 | DSCR3 Down syndrome critical region | 4191 |
| | protein 3 | |
| O60232_C125 | SSSCA1 Sjoegren | 4192 |
| | syndrome/scleroderma autoantigen 1 | |
| O95619_C210 | YEATS4 YEATS domain-containing | 4193 |
| | protein 4 | |
| P57772_C93 | EEFSEC Selenocysteine-specific | 4194 |
| | elongation factor | |
| P13489_C248 | RNH1 Ribonuclease inhibitor | 4195 |
| P13489_C362 | RNH1 Ribonuclease inhibitor | 4196 |
| Q8ND83_C449 | SLAIN1 SLAIN motif-containing | 4197 |
| | protein 1 | |
| Q9BUK6_C411 | MSTO1 Protein misato homolog 1 | 4198 |
| Q12888_C329 | TP53BP1 Tumor suppressor p53- | 4199 |
| | binding protein 1 | |
| Q15910_C14 | EZH2 Histone-lysine N- | 4200 |
| | methyltransferase EZH2 | |
| Q99832_C370 | CCT7 T-complex protein 1 subunit eta | 4201 |
| Q9BZH6_C363 | WDR11 WD repeat-containing protein | 4202 |
| | 11 | |
| Q07864_C1935 | POLE DNA polymerase epsilon | 4203 |
| | catalytic subunit A | |
| Q92974_C383 | ARHGEF2 Rho guanine nucleotide | 4204 |
| | exchange factor 2 | |
| Q9BTE3_C636 | MCMBP Mini-chromosome | 4205 |
| | maintenance complex-binding protein | |
| P54578_C203 | USP14 Ubiquitin carboxyl-terminal | 4206 |
| | hydrolase 14 | |
| P54578_C415 | USP14 Ubiquitin carboxyl-terminal | 4207 |
| | hydrolase 14 | |
| O75995_C152 | SASH3 SAM and SH3 domain- | 4208 |
| | containing protein 3 | |
| O95425_C671 | SVIL Supervillin | 4209 |
| Q9UHI6_C577 | DDX20 Probable ATP-dependent RNA | 4210 |
| | helicase DDX20 | |
| Q9H9Q2_C240 | COPS7B COP9 signalosome complex | 4211 |
| | subunit 7b | |
| P62191_C58 | PSMC1 26S protease regulatory subunit | 4212 |
| | 4 | |
| P62195_C363 | PSMC5 26S protease regulatory subunit | 4213 |
| | 8 | |
| P11413_C294 | G6PD Glucose-6-phosphate 1- | 4214 |
| | dehydrogenase | |
| P11142_C267 | HSPA8 Heat shock cognate 71 kDa | 4215 |
| | protein | |
| Q9NQX3_C419 | GPHN Gephyrin | 4216 |
| P61081_C47 | UBE2M NEDD8-conjugating enzyme | 4217 |
| | Ubc12 | |
| Q9Y6W3_C767 | CAPN7 Calpain-7 | 4218 |
| Q01844_C524 | EWSR1 RNA-binding protein EWS | 4219 |
| Q9Y2Q3_C27 | GSTK1 Glutathione S-transferase | 4220 |
| | kappa 1 | |
| O60825_C28 | PFKFB2 6-phosphofructo-2- | 4221 |
| | kinase/fructose-2,6-bisphosphatase | |
| Q9GZV4_C73 | EIF5A2 Eukaryotic translation | 4222 |
| | initiation factor 5A-2 | |
| Q9NXV6_C24 | CDKN2AIP CDKN2A-interacting | 4223 |
| | protein | |
| Q9UID3_C316 | VPS51 Vacuolar protein sorting- | 4224 |
| | associated protein 51 homolog | |
| Q99829_C30 | CPNE1 Copine-1 | 4225 |
| Q5VT52_C416 | RPRD2 Regulation of nuclear pre- | 4226 |
| | mRNA domain-containing protein 2 | |
| P22314_C906 | UBA1 Ubiquitin-like modifier- | 4227 |
| | activating enzyme 1 | |
| Q9UKV3_C691 | ACIN1 Apoptotic chromatin | 4228 |
| | condensation inducer in the nucleus | |
| Q86U28_C144 | ISCA2 Iron-sulfur cluster assembly 2 | 4229 |
| | homolog, mitochondrial | |
| Q15814_C184 | TBCC Tubulin-specific chaperone C | 4230 |
| P42695_C1484 | NCAPD3 Condensin-2 complex subunit | 4231 |
| | D3 | |
| Q14493_C72 | SLBP Histone RNA hairpin-binding | 4232 |
| | protein | |
| Q14258_C498 | TRIM25 E3 ubiquitin/ISG15 ligase | 4233 |
| | TRIM25 | |
| Q9H3U1_C384 | UNC45A Protein unc-45 homolog A | 4234 |
| O43374_C396 | RASA4 Ras GTPase-activating protein | 4235 |
| | 4 | |
| P82921_C49 | MRPS21 28S ribosomal protein S21, | 4236 |
| | mitochondrial | |
| P20810_C241 | CAST Calpastatin | 4237 |
| P20810_C413 | CAST Calpastatin | 4238 |
| Q9Y5S9_C149 | RBM8A RNA-binding protein 8A | 4239 |
| P31997_C299 | CEACAM8 Carcinoembryonic antigen- | 4240 |
| | related cell adhesion molecule 8 | |
| Q9ULV4_C39 | CORO1C Coronin-1C | 4241 |
| PI1172_C174 | UMPS Uridine 5-monophosphate | 4242 |
| | synthase | |
| Q6NXE6_C297 | ARMC6 Armadillo repeat-containing | 4243 |
| | protein 6 | |
| Q92797_C578 | SYMPK Symplekin | 4244 |
| P45984_C116 | MAPK9 Mitogen-activated protein | 4245 |
| | kinase 9 | |
| P50995_C294 | ANXA11 Annexin A11 | 4246 |
| O95376_C161 | ARIH2 E3 ubiquitin-protein ligase | 4247 |
| | ARIH2 | |
| B0V043_C381 | VARS Valyl-tRNA synthetase | 4248 |
| B0V043_C444 | VARS Valyl-tRNA synthetase | 4249 |
| P49848_C130 | TAF6 Transcription initiation factor | 4250 |
| | TFIID subunit 6 | |
| Q86UV5_C850 | USP48 Ubiquitin carboxyl-terminal | 4251 |
| | hydrolase 48 | |
| Q9Y2H0_C726 | DLGAP4 Disks large-associated protein | 4252 |
| | 4 | |
| Q92947_C176 | GCDH Glutaryl-CoA dehydrogenase, | 4253 |
| | mitochondrial | |
| Q9UKW4_C800 | VAV3 Guanine nucleotide exchange | 4254 |
| | factor VAV3 | |
| P35610_C92 | SOAT1 Sterol O-acyltransferase 1 | 4255 |
| Q14318_C274 | FKBP8 Peptidyl-prolyl cis-trans | 4256 |
| | isomerase FKBP8 | |
| Q14315_C2154 | FLNC Filamin-C | 4257 |
| P22234_C281 | PAICS Multifunctional protein ADE2 | 4258 |
| P22234_C423 | PAICS Multifunctional protein ADE2 | 4259 |
| Q969Z0_C370 | TBRG4 Protein TBRG4 | 4260 |
| Q8WUM4_C40 | PDCD6IP Programmed cell death 6- | 4261 |
| | interacting protein | |
| Q9H9A6_C264 | LRRC40 Leucine-rich repeat- | 4262 |
| | containing protein 40 | |
| P33764_C30 | S100A3 Protein S100-A3 | 4263 |
| P38936_C117 | CDKN1A Cyclin-dependent kinase | 4264 |
| | inhibitor 1 | |
| Q9UPU5_C1362 | USP24 Ubiquitin carboxyl-terminal | 4265 |
| | hydrolase 24 | |
| Q8IWD4_C81 | CCDC117 Coiled-coil domain- | 4266 |
| | containing protein 117 | |
| Q96GM8_C80 | TOE1 Target of EGR1 protein 1 | 4267 |
| Q9UJY4_C347 | GGA2 ADP-ribosylation factor-binding | 4268 |
| | protein GGA2 | |
| P12236_C129 | SLC25A6 ADP/ATP translocase 3 | 4269 |
| Q9NXH9_C620 | TRMT1 tRNA (guanine(26)-N(2))- | 4270 |
| | dimethyltransferase | |
| Q7L0Y3_C123 | TRMT10C Mitochondrial ribonuclease | 4271 |
| | P protein 1 | |
| Q08211_C12 | DHX9 ATP-dependent RNA helicase A | 4272 |
| Q9Y4I1_C535 | MYO5A Unconventional myosin-Va | 4273 |
| P08240_C253 | SRPR Signal recognition particle | 4274 |
| | receptor subunit alpha | |
| Q5TFE4_C399 | NT5DC1 5-nucleotidase domain- | 4275 |
| | containing protein 1 | |
| Q8NFW8_C364 | CMAS N-acylneuraminate | 4276 |
| | cytidylyltransferase | |
| O95817_C373 | BAG3 BAG family molecular | 4277 |
| | chaperone regulator 3 | |
| Q92541_C599 | RTF1 RNA polymerase-associated | 4278 |
| | protein RTF1 homolog | |
| P52789_C375 | HK2 Hexokinase-2 | 4279 |
| P52789_C813 | HK2 Hexokinase-2 | 4280 |
| Q01469_C87 | FABP5 Fatty acid-binding protein, | 4281 |
| | epidermal | |
| O43172_C342 | PRPF4 U4/U6 small nuclear | 4282 |
| | ribonucleoprotein Prp4 | |
| O43175_C48 | PHGDH D-3-phosphoglycerate | 4283 |
| | dehydrogenase | |
| O43175_C234 | PHGDH D-3-phosphoglycerate | 4284 |
| | dehydrogenase | |
| P22059_C344 | OSBP Oxysterol-binding protein 1 | 4285 |
| Q14699_C551 | RFTN1 Raftlin | 4286 |
| Q14697_C822 | GANAB Neutral alpha-glucosidase AB | 4287 |
| Q15654_C310 | TRIP6 Thyroid receptor-interacting | 4288 |
| | protein 6 | |
| Q15654_C328 | TRIP6 Thyroid receptor-interacting | 4289 |
| | protein 6 | |
| Q15652_C684 | JMJD1C Probable JmjC domain- | 4290 |
| | containing histone demethylation | |
| | protein 2C | |
| Q9NU22_C1867 | MDN1 Midasin | 4291 |
| Q13439_C1862 | GOLGA4 Golgin subfamily A member | 4292 |
| | 4 | |
| Q9UPT8_C1302 | ZC3H4 Zinc finger CCCH domain- | 4293 |
| | containing protein 4 | |
| Q9NXG2_C169 | THUMPD1 THUMP domain- | 4294 |
| | containing protein 1 | |
| Q27J81_C1029 | INF2 Inverted formin-2 | 4295 |
| P01892_C188 | HLA-A HLA class I histocompatibility | 4296 |
| | antigen, A-2 alpha | |
| P60903_C62 | S100A10 Protein S100-A10 | 4297 |
| P51553_C236 | IDH3G Isocitrate dehydrogenase | 4298 |
| P51003_C36 | PAPOLA Poly(A) polymerase alpha | 4299 |
| P50213_C351 | IDH3A Isocitrate dehydrogenase | 4300 |
| P62873_C204 | GNB1 Guanine nucleotide-binding | 4301 |
| | protein G(I)/G(S)/G(T) | |
| Q15003_C255 | NCAPH Condensin complex subunit 2 | 4302 |
| O00764_C273 | PDXK Pyridoxal kinase | 4303 |
| Q9BZL6_C217 | PRKD2 Serine/threonine-protein kinase | 4304 |
| | D2 | |
| O75534_C506 | CSDE1 Cold shock domain-containing | 4305 |
| | protein E1 | |
| Q9C0B1_C397 | FTO Alpha-ketoglutarate-dependent | 4306 |
| | dioxygenase FTO | |
| O75223_C42 | GGCT Gamma- | 4307 |
| | glutamylcyclotransferase | |
| Q13155_C168 | AIMP2 Aminoacyl tRNA synthase | 4308 |
| | complex-interacting multifunctional | |
| | protein 2 | |
| Q14204_C3389 | DYNC1H1 Cytoplasmic dynein 1 | 4309 |
| | heavy chain 1 | |
| Q8WXI9_C409 | GATAD2B Transcriptional repressor | 4310 |
| | p66-beta | |
| P07814_C1301 | EPRS Bifunctional glutamate/proline-- | 4311 |
| | tRNA ligase | |
| P22102_C62 | GART Trifunctional purine | 4312 |
| | biosynthetic protein adenosine | |
| O75643_C428 | SNRNP200 U5 small nuclear | 4313 |
| | ribonucleoprotein 200 kDa helicase | |
| Q9ULL5_C173 | PRR12 Proline-rich protein 12 | 4314 |
| Q14738_C484 | PPP2R5D Serine/threonine-protein | 4315 |
| | phosphatase 2A 56 kDa regulatory | |
| P50851_C1843 | LRBA Lipopolysaccharide-responsive | 4316 |
| | and beige-like anchor protein | |
| Q9H668_C8 | OBFC1 CST complex subunit STN1 | 4317 |
| Q15643_C1329 | TRIP11 Thyroid receptor-interacting | 4318 |
| | protein 11 | |
| Q13422_C394 | IKZF1 DNA-binding protein Ikaros | 4319 |
| P48739_C94 | PITPNB Phosphatidylinositol transfer | 4320 |
| | protein beta isoform | |
| Q9UJM3_C378 | ERRFI1 ERBB receptor feedback | 4321 |
| | inhibitor 1 | |
| P62136_C140 | PPP1CA Serine/threonine-protein | 4322 |
| | phosphatase PP1-alpha catalytic | |
| P13674_C528 | P4HA1 Prolyl 4-hydroxylase subunit | 4323 |
| | alpha-1 | |
| Q9NVX2_C280 | NLE1 Notchless protein homolog 1 | 4324 |
| Q6IA86_C68 | ELP2 Elongator complex protein 2 | 4325 |
| P45954_C308 | ACADSB Short/branched chain | 4326 |
| | specific acyl-CoA dehydrogenase | |
| O94921_C100 | CDK14 Cyclin-dependent kinase 14 | 4327 |
| Q3KQU3_C361 | MAP7D1 MAP7 domain-containing | 4328 |
| | protein 1 | |
| Q8IVM0_C85 | CCDC50 Coiled-coil domain- | 4329 |
| | containing protein 50 | |
| P52630_C74 | STAT2 Signal transducer and activator | 4330 |
| | of transcription 2 | |
| P52630_C529 | STAT2 Signal transducer and activator | 4331 |
| | of transcription 2 | |
| P42704_C571 | LRPPRC Leucine-rich PPR motif- | 4332 |
| | containing protein, mitochondrial | |
| P42704_C1043 | LRPPRC Leucine-rich PPR motif- | 4333 |
| | containing protein, mitocho | |
| Q99598_C202 | TSNAX Translin-associated protein X | 4334 |
| P34932_C376 | HSPA4 Heat shock 70 kDa protein 4 | 4335 |
| Q14980_C65 | NUMA1 Nuclear mitotic apparatus | 4336 |
| | protein 1 | |
| Q14980_C1367 | NUMA1 Nuclear mitotic apparatus | 4337 |
| | protein 1 | |
| Q9BYV8_C274 | CEP41 Centrosomal protein of 41 kDa | 4338 |
| P51970_C66 | NDUFA8 NADH dehydrogenase | 4339 |
| Q01433_C230 | AMPD2 AMP deaminase 2 | 4340 |
| P55212_C68 | CASP6 Caspase-6 | 4341 |
| Q5SW79_C235 | CEP170 Centrosomal protein of 170 | 4342 |
| | kDa | |
| Q9NQG5_C234 | RPRD1B Regulation of nuclear pre- | 4343 |
| | mRNA domain-containing protein | |
| O00743_C265 | PPP6C Serine/threonine-protein | 4344 |
| | phosphatase 6 catalytic subunit | |
| PI0515_C586 | DLAT Dihydrolipoyllysine-residue | 4345 |
| | acetyltransferase component of | |
| | pyruvate dehydrogenase complex, | |
| | mitochondiral | |
| P28340_C360 | POLD1 DNA polymerase delta | 4346 |
| | catalytic subunit | |
| P28340_C1029 | POLD1 DNA polymerase delta | 4347 |
| | catalytic subunit | |
| Q86VP6_C179 | CAND1 Cullin-associated NEDD8- | 4348 |
| | dissociated protein 1 | |
| Q86VP6_C954 | CAND1 Cullin-associated NEDD8- | 4349 |
| | dissociated protein 1 | |
| P54886_C486 | ALDH18A1 Delta-1-pyrroline-5- | 4350 |
| | carboxylate synthase | |
| P54886_C546 | ALDH18A1 Delta-l-pyrroline-5- | 4351 |
| | carboxylate synthase | |
| Q8N0X7_C288 | SPG20 Spartin | 4352 |
| Q8TB52_C723 | FBXO30 F-box only protein 30 | 4353 |
| Q14152_C404 | EIF3A Eukaryotic translation initiation | 4354 |
| | factor 3 subunit | |
| P21281_C112 | ATP6V1B2 V-type proton ATPase | 4355 |
| | subunit B, brain isoform | |
| P21281_C207 | ATP6V1B2 V-type proton ATPase | 4356 |
| | subunit B, brain isoform | |
| O75116_C804 | ROCK2 Rho-associated protein kinase | 4357 |
| | 2 | |
| Q5UIP0_C2169 | RIF1 Telomere-associated protein RIF1 | 4358 |
| Q9BW27_C515 | NUP85 Nuclear pore complex protein | 4359 |
| | Nup85 | |
| P55199_C70 | ELL RNA polymerase II elongation | 4360 |
| | factor ELL | |
| Q8WXE1_C74 | ATRIP ATR-interacting protein | 4361 |
| O14526_C571 | FCHO1 FCH domain only protein 1 | 4362 |
| Q9NSD9_C362 | FARSB Phenylalanine--tRNA ligase | 4363 |
| | beta subunit | |
| Q8WUW1_C43 | BRK1 Protein BRICK1 | 4364 |
| Q12874_C145 | SF3A3 Splicing factor 3A subunit 3 | 4365 |
| Q9Y6A5_C20 | TACC3 Transforming acidic coiled- | 4366 |
| | coil-containing protein | |
| Q9Y6A5_C426 | TACC3 Transforming acidic coiled- | 4367 |
| | coil-containing protein | |
| Q92878_C133 | RAD50 DNA repair protein RAD50 | 4368 |
| Q9BXK1_C233 | KLF16 Krueppel-like factor 16 | 4369 |
| Q68CZ2_C842 | TNS3 Tensin-3 | 4370 |
| O75616_C86 | ERAL1 GTPase Era, mitochondrial | 4371 |
| P18031_C32 | PTPN1 Tyrosine-protein phosphatase | 4372 |
| | non-receptor type 1 | |
| Q9UHB6_C316 | LIMA1 LIM domain and actin-binding | 4373 |
| | protein 1 | |
| Q9UHB6_C414 | LIMA1 LIM domain and actin-binding | 4374 |
| | protein 1 | |
| Q9NZ52_C396 | GGA3 ADP-ribosylation factor-binding | 4375 |
| | protein GGA3 | |
| Q9UBT2_C185 | UBA2 SUMO-activating enzyme | 4376 |
| | subunit 2 | |
| Q15723_C470 | ELF2 ETS-related transcription factor | 4377 |
| | Elf-2 | |
| Q9H5V9_C11 | CXorf56 UPF0428 protein CXorf56 | 4378 |
| O14980_C199 | XPO1 Exportin-1 | 4379 |
| P15104_C53 | GLUL Glutamine synthetase | 4380 |
| P13796_C206 | LCP1 Plastin-2 | 4381 |
| O95071_C2084 | UBR5 E3 ubiquitin-protein ligase | 4382 |
| | UBR5 | |
| Q6XZF7_C909 | DNMBP Dynamin-binding protein | 4383 |
| Q9NXR7_C34 | BRE BRCA1-A complex subunit BRE | 4384 |
| Q9NQW6_C61 | ANLN Actin-binding protein anillin | 4385 |
| Q9NQW6_C234 | ANLN Actin-binding protein anillin | 4386 |
| Q6P587_C129 | FAHD1 Acylpyruvase FAHD1, | 4387 |
| | mitochondrial | |
| Q8NC96_C162 | NECAP1 Adaptin ear-binding coat- | 4388 |
| | associated protein 1 | |
| Q00653_C738 | NFKB2 Nuclear factor NF-kappa-B | 4389 |
| | p100 subunit | |
| Q86W56_C603 | PARG Poly(ADP-ribose) | 4390 |
| | glycohydrolase | |
| P39748_C182 | FEN1 Flap endonuclease 1 | 4391 |
| Q9Y3Z3_C320 | SAMHD1 SAM domain and HD | 4392 |
| | domain-containing protein 1 | |
| Q13045_C1265 | FLH Protein flightless-1 homolog | 4393 |
| Q14558_C19 | PRPSAP1 Phosphoribosyl | 4394 |
| | pyrophosphate synthase-associated | |
| | protein 1 | |
| O75592_C3152 | MYCBP2 Probable E3 ubiquitin- | 4395 |
| | protein ligase MYCBP2 | |
| Q8IUR0_C40 | TRAPPC5 Trafficking protein particle | 4396 |
| | complex subunit 5 | |
| Q16822_C92 | PCK2 Phosphoenolpyruvate | 4397 |
| | carboxykinase | |
| Q53H96_C129 | PYCRL Pyrroline-5-carboxylate | 4398 |
| | reductase 3 | |
| Q96F24_C126 | NRBF2 Nuclear receptor-binding factor | 4399 |
| | 2 | |
| H3BQ06_C89 | Uncharacterized protein | 4400 |
| Q06546_C421 | GABPA GA-binding protein alpha | 4401 |
| | chain | |
| P56192_C309 | MARS Methionine--tRNA ligase, | 4402 |
| | cytoplasmic | |
| Q9Y485_C1419 | DMXL1 DmX-like protein 1 | 4403 |
| Q00839_C408 | HNRNPU Heterogeneous nuclear | 4404 |
| | ribonucleoprotein U | |
| Q92499_C631 | DDX1 ATP-dependent RNA helicase | 4405 |
| | DDX1 | |
| O43663_C531 | PRC1 Protein regulator of cytokinesis 1 | 4406 |
| O14879_C239 | IFIT3 Interferon-induced protein with | 4407 |
| | tetratricopeptide | |
| P36969_C134 | GPX4 Phospholipid hydroperoxide | 4408 |
| | glutathione peroxidase, | |
| P12081_C235 | HARS Histidine--tRNA ligase, | 4409 |
| | cytoplasmic | |
| P50914_C54 | RPL14 60S ribosomal protein L14 | 4410 |
| Q9H0D6_C547 | XRN2 5-3 exoribonuclease 2 | 4411 |
| Q14C86_C1129 | GAPVD1 GTPase-activating protein | 4412 |
| | and VPS9 domain-containi | |
| Q9BXF6_C106 | RAB11FIP5 Rab11 family-interacting | 4413 |
| | protein 5 | |
| A6NHR9_C1856 | SMCHD1 Structural maintenance of | 4414 |
| | chromosomes flexible hin | |
| Q9BW92_C322 | TARS2 Threonine--tRNA ligase, | 4415 |
| | mitochondrial | |
| P83916_C156 | CBX1 Chromobox protein homolog 1 | 4416 |
| O00410_C229 | IPO5 Importin-5 | 4417 |
| Q5VUA4_C1681 | ZNF318 Zinc finger protein 318 | 4418 |
| Q96SY0_C193 | C15orf44 UPF0464 protein C15orf44 | 4419 |
| Q9NS18_C77 | GLRX2 Glutaredoxin-2, mitochondrial | 4420 |
| P46109_C44 | CRKL Crk-like protein | 4421 |
| Q14289_C677 | PTK2B Protein-tyrosine kinase 2-beta | 4422 |
| Q14289_C972 | PTK2B Protein-tyrosine kinase 2-beta | 4423 |
| O75718_C317 | CRTAP Cartilage-associated protein | 4424 |
| Q86Y37_C94 | CACUL1 CDK2-associated and cullin | 4425 |
| | domain-containing protein | |
| P16144_C1553 | ITGB4 Integrin beta-4 | 4426 |
| Q9BY42_C51 | C20orf43 UPF0549 protein C20orf43 | 4427 |
| Q8ND56_C375 | LSM14A Protein LSM14 homolog A | 4428 |
| P40121_C165 | CAPG Macrophage-capping protein | 4429 |
| P78347_C745 | GTF2I General transcription factor II-I | 4430 |
| P78347_C903 | GTF2I General transcription factor II-I | 4431 |
| P16885_C496 | PLCG2 1-phosphatidylinositol 4,5- | 4432 |
| | bisphosphate phosphodiese | |
| Q9H814_C87 | PHAX Phosphorylated adapter RNA | 4433 |
| | export protein | |
| P29401_C41 | TKT Transketolase | 4434 |
| P35249_C141 | RFC4 Replication factor C subunit 4 | 4435 |
| Q92835_C956 | INPP5D Phosphatidylinositol 3,4,5- | 4436 |
| | trisphosphate 5-phosphatase | |
| Q9Y6E0_C375 | STK24 Serine/threonine-protein kinase | 4437 |
| | 24 | |
| O75380_C87 | NDUFS6 NADH dehydrogenase | 4438 |
| Q9Y4G6_C1954 | TLN2 Talin-2 | 4439 |
| Q9NYG5_C7 | ANAPC11 Anaphase-promoting | 4440 |
| | complex subunit 11 | |
| Q9UPN3_C5131 | MACF1 Microtubule-actin cross- | 4441 |
| | linking factor 1, isoforms | |
| P53634_C448 | CTSC Dipeptidyl peptidase 1 | 4442 |
| Q96HA7_C830 | TONSL Tonsoku-like protein | 4443 |
| A5YKK6_C677 | CNOT1 CCR4-NOT transcription | 4444 |
| | complex subunit 1 | |
| Q9BWU0_C125 | SLC4A1AP Kanadaptin | 4445 |
| Q9BWU0_C730 | SLC4A1AP Kanadaptin | 4446 |
| P40763_C718 | STAT3 Signal transducer and activator | 4447 |
| | of transcription 3 | |
| Q96RU3_C555 | FNBP1 Formin-binding protein 1 | 4448 |
| Q96HS1_C168 | PGAM5 Serine/threonine-protein | 4449 |
| | phosphatase PGAM5, mitochondrial | |
| Q6YN16_C136 | HSDL2 Hydroxysteroid | 4450 |
| | dehydrogenase-like protein 2 | |
| P05067_C98 | APP Amyloid beta A4 protein | 4451 |
| Q96RE7_C85 | NACC1 Nucleus accumbens-associated | 4452 |
| | protein 1 | |
| P49959_C336 | MRE11A Double-strand break repair | 4453 |
| | protein MRE11A | |
| Q01813_C232 | PFKP 6-phosphofructokinase type C | 4454 |
| P49792_C348 | RANBP2 E3 SUMO-protein ligase | 4455 |
| | RanBP2 | |
| P49792_C1296 | RANBP2 E3 SUMO-protein ligase | 4456 |
| | RanBP2 | |
| Q93008_C1566 | USP9X Probable ubiquitin carboxyl- | 4457 |
| | terminal hydrolase FAF | |
| Q9BY89_C817 | KIAA1671 Uncharacterized protein | 4458 |
| | KIAA1671 | |
| Q5SRE5_C1445 | NUP188 Nucleoporin NUP188 | 4459 |
| | homolog | |
| Q92665_C356 | MRPS31 28S ribosomal protein S31, | 4460 |
| | mitochondrial | |
| P18669_C55 | PGAM1 Phosphoglycerate mutase 1 | 4461 |
| Q9Y4P8_C70 | WIPI2 WD repeat domain | 4462 |
| | phosphoinositide-interacting protein | |
| Q9Y4P1_C189 | ATG4B Cysteine protease ATG4B | 4463 |
| Q9BZD4_C285 | NUF2 Kinetochore protein Nuf2 | 4464 |
| Q01581_C406 | HMGCS1 Hydroxymethylglutaryl-CoA | 4465 |
| | synthase, cytoplasmic | |
| P78344_C677 | EIF4G2 Eukaryotic translation | 4466 |
| | initiation factor 4 gamma 2 | |
| Q92609_C753 | TBC1D5 TBC1 domain family member | 4467 |
| | 5 | |
| Q9BSJ8_C890 | ESYT1 Extended synaptotagmin-1 | 4468 |
| P46013_C226 | MKI67 Antigen KI-67 | 4469 |
| P46013_C1373 | MKI67 Antigen KI-67 | 4470 |
| P46013_C1479 | MKI67 Antigen KI-67 | 4471 |
| P17858_C708 | PFKL 6-phosphofructokinase, liver | 4472 |
| | type | |
| Q96PK6_C31 | RBM14 RNA-binding protein 14 | 4473 |
| P53602_C133 | MVD Diphosphomevalonate | 4474 |
| | decarboxylase | |
| Q8WXH0_C1032 | SYNE2 Nesprin-2 | 4475 |
| Q14137_C108 | BOP1 Ribosome biogenesis protein | 4476 |
| | BOP1 | |
| P23368_C481 | ME2 NAD-dependent malic enzyme, | 4477 |
| | mitochondrial | |
| P51692_C688 | STAT5B Signal transducer and | 4478 |
| | activator of transcription 5 | |
| Q8TD19_C890 | NEK9 Serine/threonine-protein kinase | 4479 |
| | Nek9 | |
| Q14008_C1113 | CKAP5 Cytoskeleton-associated | 4480 |
| | protein 5 | |
| Q14008_C1946 | CKAP5 Cytoskeleton-associated | 4481 |
| | protein 5 | |
| Q9UJU6_C67 | DBNL Drebrin-like protein | 4482 |
| Q9UPQ0_C182 | LIMCH1 LIM and calponin homology | 4483 |
| | domains-containing protein | |
| Q15424_C362 | SAFB Scaffold attachment factor B1 | 4484 |
| O95757_C290 | HSPA4L Heat shock 70 kDa protein 4L | 4485 |
| P21333_C623 | FLNA Filamin-A | 4486 |
| P51610_C326 | HCFC1 Host cell factor 1 | 4487 |
| Q9H6E5_C501 | TUT1 Speckle targeted PIP5K1A- | 4488 |
| | regulated poly(A) polymer | |
| O00154_C100 | ACOT7 Cytosolic acyl coenzyme A | 4489 |
| | thioester hydrolase | |
| O95347_C800 | SMC2 Structural maintenance of | 4490 |
| | chromosomes protein 2 | |
| Q96SZ5_C239 | ADO 2-aminoethanethiol dioxygenase | 4491 |
| P18615_C297 | RDBP Negative elongation factor E | 4492 |
| Q93034_C112 | CUL5 Cullin-5 | 4493 |
| O43913_C171 | ORC5 Origin recognition complex | 4494 |
| | subunit 5 | |
| P01033_C189 | TIMP1 Metalloproteinase inhibitor 1 | 4495 |
| Q92616_C1362 | GCN1L1 Translational activator GCN1 | 4496 |
| Q92616_C1781 | GCN1L1 Translational activator GCN1 | 4497 |
| Q99661_C154 | KIF2C Kinesin-like protein KIF2C | 4498 |
| O60701_C196 | UGDH UDP-glucose 6-dehydrogenase | 4499 |
| Q9UH65_C279 | SWAP70 Switch-associated protein 70 | 4500 |
| Q96FS4_C446 | SIPA1 Signal-induced proliferation- | 4501 |
| | associated protein 1 | |
| P49327_C1186 | FASN Fatty acid synthase | 4502 |
| P49327_C1759 | FASN Fatty acid synthase | 4503 |
| P49327_C2292 | FASN Fatty acid synthase | 4504 |
| Q05655_C280 | PRKCD Protein kinase C delta type | 4505 |
| Q05655_C344 | PRKCD Protein kinase C delta type | 4506 |
| Q9BQE9_C189 | BCL7B B-cell CLL/lymphoma 7 | 4507 |
| | protein family member B | |
| Q8N1G0_C91 | ZNF687 Zinc finger protein 687 | 4508 |
| P13489_C45 | RNH1 Ribonuclease inhibitor | 4509 |
| P48444_C479 | ARCN1 Coatomer subunit delta | 4510 |
| Q96BN8_C129 | FAM105B Protein FAM105B | 4511 |
| Q9UKU7_C159 | ACAD8 Isobutyryl-CoA | 4512 |
| | dehydrogenase, mitochondrial | |
| Q9H2L5_C249 | RASSF4 Ras association domain- | 4513 |
| | containing protein 4 | |
| Q12888_0178 | TP53BP1 Tumor suppressor p53- | 4514 |
| | binding protein 1 | |
| P29034_C94 | S100A2 Protein S100-A2 | 4515 |
| Q9UQ35_C785 | SRRM2 Serine/arginine repetitive | 4516 |
| | matrix protein 2 | |
| O00329_C474 | PIK3CD Phosphatidylinositol 4,5- | 4517 |
| | bisphosphate 3-kinase catalytic | |
| Q9Y2Y0_C149 | ARL2BP ADP-ribosylation factor-like | 4518 |
| | protein 2-binding protein | |
| O15357_C1121 | INPPL1 Phosphatidylinositol 3,4,5- | 4519 |
| | trisphosphate 5-phosphatase 2 | |
| Q99832_C345 | CCT7 T-complex protein 1 subunit eta | 4520 |
| Q9Y4D8_C1370 | HECTD4 Probable E3 ubiquitin-protein | 4521 |
| | ligase HECTD4 | |
| Q6KC79_C419 | NIPBL Nipped-B-like protein | 4522 |
| P04179_C164 | SOD2 Superoxide dismutase | 4523 |
| Q96RG2_C15 | PASK PAS domain-containing | 4524 |
| | serine/threonine-protein kinase | |
| Q14061_C24 | COX17 Cytochrome c oxidase copper | 4525 |
| | chaperone | |
| Q96DC7_C14 | TMCO6 Transmembrane and coiled- | 4526 |
| | coil domain-containing protein | |
| P20248_C327 | CCNA2 Cyclin-A2 | 4527 |
| Q02878_C44 | RPL6 60S ribosomal protein L6 | 4528 |
| O14773_C365 | TPP1 Tripeptidyl-peptidase 1 | 4529 |
| P61086_C92 | UBE2K Ubiquitin-conjugating enzyme | 4530 |
| | E2 K | |
| P31146_C332 | CORO1A Coronin-1A | 4531 |
| Q9BR76_C153 | CORO1B Coronin-1B | 4532 |
| Q96QC0_C463 | PPP1R10 Serine/threonine-protein | 4533 |
| | phosphatase 1 regulatory | |
| Q5VT52_C1071 | RPRD2 Regulation of nuclear pre- | 4534 |
| | mRNA domain-containing p | |
| P78527_C373 | PRKDC DNA-dependent protein kinase | 4535 |
| | catalytic subunit | |
| Q96A35_C58 | MRPL24 39S ribosomal protein L24, | 4536 |
| | mitochondrial | |
| Q14498_C303 | RBM39 RNA-binding protein 39 | 4537 |
| Q8N556_C157 | AFAP1 Actin filament-associated | 4538 |
| | protein 1 | |
| Q68CP9_C711 | ARID2 AT-rich interactive domain- | 4539 |
| | containing protein 2 | |
| Q9H3U1_C663 | UNC45A Protein unc-45 homolog A | 4540 |
| Q99567_C595 | NUP88 Nuclear pore complex protein | 4541 |
| | Nup88 | |
| Q14789_C1713 | GOLGB1 Golgin subfamily B member | 4542 |
| | 1 | |
| P20810_C661 | CAST Calpastatin | 4543 |
| P07203_C115 | GPX1 Glutathione peroxidase 1 | 4544 |
| O60216_C35 | RAD21 Double-strand-break repair | 4545 |
| | protein rad21 homolog | |
| O60216_C513 | RAD21 Double-strand-break repair | 4546 |
| | protein rad21 homolog | |
| Q9ULV4_C190 | CORO1C Coronin-1C | 4547 |
| P30153_C174 | PPP2R1A Serine/threonine-protein | 4548 |
| | phosphatase 2A 65 kDa regulatory | |
| | subunit A | |
| P30153_C329 | PPP2R1A Serine/threonine-protein | 4549 |
| | phosphatase 2A 65 kDa regulatory | |
| | subunit A | |
| Q9UEW8_C99 | STK39 STE20/SPS1-related proline- | 4550 |
| | alanine-rich protein kinase | |
| Q13574_C265 | DGKZ Diacylglycerol kinase zeta | 4551 |
| O00233_C216 | PSMD9 26S proteasome non-ATPase | 4552 |
| | regulatory subunit 9 | |
| P05023_C705 | ATP1A1 Sodium/potassium- | 4553 |
| | transporting ATPase subunit alpha | |
| P45985_C266 | MAP2K4 Dual specificity mitogen- | 4554 |
| | activated protein kinase | |
| P45983_C245 | MAPK8 Mitogen-activated protein | 4555 |
| | kinase 8 | |
| Q9P2R7_C320 | SUCLA2 Succinyl-CoA ligase | 4556 |
| P31040_C89 | SDHA Succinate dehydrogenase | 4557 |
| O76071_C234 | CIAO1 Probable cytosolic iron-sulfur | 4558 |
| | protein assembly protein CIAO1 | |
| O95372_C56 | LYPLA2 Acyl-protein thioesterase 2 | 4559 |
| Q04864_C524 | REL Proto-oncogene c-Rel | 4560 |
| Q96CD2_C173 | PPCDC Phosphopantothenoylcysteine | 4561 |
| | decarboxylase | |
| A6NC98_C1222 | CCDC88B Coiled-coil domain- | 4562 |
| | containing protein 88B | |
| Q10570_C1020 | CPSF1 Cleavage and polyadenylation | 4563 |
| | specificity factor subunit 1 | |
| P43378_C506 | PTPN9 Tyrosine-protein phosphatase | 4564 |
| | non-receptor type 9 | |
| Q15021_C816 | NCAPD2 Condensin complex subunit 1 | 4565 |
| Q8TEW0_C6 | PARD3 Partitioning defective 3 | 4566 |
| | homolog | |
| O94925_C500 | GLS Glutaminase kidney isoform, | 4567 |
| | mitochondrial | |
| Q9Y3B8_C137 | REXO2 Oligoribonuclease, | 4568 |
| | mitochondrial | |
| Q96LD4_C347 | TRIM47 Tripartite motif-containing | 4569 |
| | protein 47 | |
| Q9H081_C104 | MIS12 Protein MIS12 homolog | 4570 |
| P54687_C293 | BCAT1 Branched-chain-amino-acid | 4571 |
| | aminotransferase, cytosol | |
| Q8WUM4_C524 | PDCD6IP Programmed cell death 6- | 4572 |
| | interacting protein | |
| Q8WUM4_C691 | PDCD6IP Programmed cell death 6- | 4573 |
| | interacting protein | |
| Q9H9A7_C366 | RMI1 RecQ-mediated genome | 4574 |
| | instability protein 1 | |
| Q13315_C2021 | ATM Serine-protein kinase ATM | 4575 |
| P11766_C174 | ADH5 Alcohol dehydrogenase class-3 | 4576 |
| Q96EI5_C34 | TCEAL4 Transcription elongation | 4577 |
| | factor A protein-like 4 | |
| Q9NR45_C184 | NANS Sialic acid synthase | 4578 |
| Q13542_C35 | EIF4EBP2 Eukaryotic translation | 4579 |
| | initiation factor 4E-binding | |
| Q13547_C151 | HDAC1 Histone deacetylase 1 | 4580 |
| Q8NF50_C939 | DOCK8 Dedicator of cytokinesis | 4581 |
| | protein 8 | |
| P09110_C218 | ACAA1 3-ketoacyl-CoA thiolase, | 4582 |
| | peroxisomal | |
| O60841_C749 | EIF5B Eukaryotic translation initiation | 4583 |
| | factor 5B | |
| Q15306_C223 | IRF4 Interferon regulatory factor 4 | 4584 |
| Q96S59_C513 | RANBP9 Ran-binding protein 9 | 4585 |
| O60443_C180 | DFNA5 Non-syndromic hearing | 4586 |
| | impairment protein 5 | |
| P08240_C621 | SRPR Signal recognition particle | 4587 |
| | receptor subunit alpha | |
| Q9NRR5_C29 | UBQLN4 Ubiquilin-4 | 4588 |
| Q92769_C274 | HDAC2 Histone deacetylase 2 | 4589 |
| O43823_C85 | AKAP8 A-kinase anchor protein 8 | 4590 |
| Q9Y3C7_C93 | MED31 Mediator of RNA polymerase | 4591 |
| | II transcription subunit | |
| Q7Z7A3_C210 | CTU1 Cytoplasmic tRNA 2-thiolation | 4592 |
| | protein 1 | |
| Q14696_C180 | MESDC2 LDLR chaperone MESD | 4593 |
| P19367_C628 | HK1 Hexokinase-1 | 4594 |
| Q13557_C373 | CAMK2D Calcium/calmodulin- | 4595 |
| | dependent protein kinase type I | |
| Q9HB71_C154 | CACYBP Calcyclin-binding protein | 4596 |
| O00148_C299 | DDX39A ATP-dependent RNA | 4597 |
| | helicase DDX39A | |
| Q9BW19_C509 | KIFC1 Kinesin-like protein KIFC1 | 4598 |
| P48643_C440 | CCT5 T-complex protein 1 subunit | 4599 |
| | epsilon | |
| Q03164_C2841 | MLL Histone-lysine N- | 4600 |
| | methyltransferase MLL | |
| Q9H0L4_C441 | CSTF2T Cleavage stimulation factor | 4601 |
| | subunit 2 tau variant | |
| P51553_C148 | IDH3G Isocitrate dehydrogenase | 4602 |
| Q9Y678_C813 | COPG1 Coatomer subunit gamma-1 | 4603 |
| Q9UBB4_C405 | ATXN10 Ataxin-10 | 4604 |
| Q9C0C2_C1114 | TNKS1BP1 182 kDa tankyrase-1- | 4605 |
| | binding protein | |
| Q9C0C2_C1175 | TNKS1BP1 182 kDa tankyrase-1- | 4606 |
| | binding protein | |
| P09651_C43 | HNRNPA1 Heterogeneous nuclear | 4607 |
| | ribonucleoprotein A1 | |
| Q96R06_C307 | SPAG5 Sperm-associated antigen 5 | 4608 |
| Q8NFH8_C380 | REPS2 RalBP1-associated Eps domain- | 4609 |
| | containing protein 2 | |
| Q14204_C3325 | DYNC1H1 Cytoplasmic dynein 1 | 4610 |
| | heavy chain 1 | |
| Q14204_C4121 | DYNC1H1 Cytoplasmic dynein 1 | 4611 |
| | heavy chain 1 | |
| O75643_C502 | SNRNP200 U5 small nuclear | 4612 |
| | ribonucleoprotein 200 kDa helicas | |
| O75643_C516 | SNRNP200 U5 small nuclear | 4613 |
| | ribonucleoprotein 200 kDa helicas | |
| Q13085_C1297 | ACACA Acetyl-CoA carboxylase 1 | 4614 |
| P04049_C27 | RAF1 RAF proto-oncogene | 4615 |
| | serine/threonine-protein kinase | |
| Q53T59_C287 | HS1BP3 HCLS 1-binding protein 3 | 4616 |
| P60468_C39 | SEC61B Protein transport protein | 4617 |
| | Sec61 subunit beta | |
| Q15019_C114 | SEPT2 Septin-2 | 4618 |
| Q15643_C1299 | TRIP11 Thyroid receptor-interacting | 4619 |
| | protein 11 | |
| Q13541_C62 | EIF4EBP1 Eukaryotic translation | 4620 |
| | initiation factor 4E-binding | |
| Q9HD42_C44 | CHMP1A Charged multivesicular body | 4621 |
| | protein 1a | |
| O15371_C327 | EIF3D Eukaryotic translation initiation | 4622 |
| | factor 3 subunit | |
| Q06187_C337 | BTK Tyrosine-protein kinase BTK | 4623 |
| P55060_C344 | CSE1L Exportin-2 | 4624 |
| Q8IUD2_C1101 | ERC1 ELKS/Rab6-interacting/CAST | 4625 |
| | family member 1 | |
| Q9UBE0_C342 | SAE1 SUMO-activating enzyme | 4626 |
| | subunit 1 | |
| Q13459_C2028 | MYO9B Unconventional myosin-IXb | 4627 |
| P41236_C86 | PPP1R2 Protein phosphatase inhibitor 2 | 4628 |
| Q6IA86_C746 | ELP2 Elongator complex protein 2 | 4629 |
| O75815_C449 | BCAR3 Breast cancer anti-estrogen | 4630 |
| | resistance protein 3 | |
| Q08J23_C599 | NSUN2 tRNA (cytosine(34)-C(5))- | 4631 |
| | methyltransferase | |
| O15231_C466 | ZNF185 Zinc finger protein 185 | 4632 |
| Q8TF05_C566 | PPP4R1 Serine/threonine-protein | 4633 |
| | phosphatase 4 regulatory | |
| Q86VQ1_C151 | GLCCI1 Glucocorticoid-induced | 4634 |
| | transcript 1 protein | |
| Q66K14_C839 | TBC1D9B TBC1 domain family | 4635 |
| | member 9B | |
| O75676_C257 | RPS6KA4 Ribosomal protein S6 kinase | 4636 |
| | alpha-4 | |
| Q01082_C2227 | SPTBN1 Spectrin beta chain, non- | 4637 |
| | erythrocytic 1 | |
| P61812_C89 | TGFB2 Transforming growth factor | 4638 |
| | beta-2 | |
| P42704_C927 | LRPPRC Leucine-rich PPR motif- | 4639 |
| | containing protein, mitochondrial | |
| Q02218_C604 | OGDH 2-oxoglutarate dehydrogenase, | 4640 |
| | mitochondrial | |
| Q99594_C368 | TEAD3 Transcriptional enhancer factor | 4641 |
| | TEF-5 | |
| P42858_C1961 | HTT Huntingtin | 4642 |
| P42858_C2971 | HTT Huntingtin | 4643 |
| P82675_C211 | MRPS5 28S ribosomal protein S5, | 4644 |
| | mitochondrial | |
| P21291_C25 | CSRP1 Cysteine and glycine-rich | 4645 |
| | protein 1 | |
| O43447_C131 | PPIH Peptidyl-prolyl cis-trans | 4646 |
| | isomerase H | |
| Q9UBR2_C132 | CTSZ Cathepsin Z | 4647 |
| P31939_C101 | ATIC Bifunctional purine biosynthesis | 4648 |
| | protein PURH | |
| Q9P2E9_C1128 | RRBP1 Ribosome-binding protein 1 | 4649 |
| Q9NWU1_C209 | OXSM 3-oxoacyl-ACP synthase, | 4650 |
| | mitochondrial | |
| O95059_C78 | RPP14 Ribonuclease P protein subunit | 4651 |
| | p14 | |
| P51570_C322 | GALK1 Galactokinase | 4652 |
| P28340_C1076 | POLD1 DNA polymerase delta | 4653 |
| | catalytic subunit | |
| Q7Z5L9_C555 | IRF2BP2 Interferon regulatory factor 2- | 4654 |
| | binding protein 2 | |
| P54886_C161 | ALDH18A1 Delta-1-pyrroline-5- | 4655 |
| | carboxylate synthase | |
| P17987_C236 | TCP1 T-complex protein 1 subunit | 4656 |
| | alpha | |
| O43143_C774 | DHX15 Putative pre-mRNA-splicing | 4657 |
| | factor ATP-dependent RN | |
| P67775_C251 | PPP2CA Serine/threonine-protein | 4658 |
| | phosphatase 2A catalytic | |
| Q92667_C102 | AKAP1 A-kinase anchor protein 1, | 4659 |
| | mitochondrial | |
| Q99615_C116 | DNAJC7 DnaJ homolog subfamily C | 4660 |
| | member 7 | |
| P04062_C165 | GBA Glucosylceramidase | 4661 |
| Q96P11_C308 | NSUN5 Putative methyltransferase | 4662 |
| | NSUN5 | |
| Q86X02_C160 | CDR2L Cerebellar degeneration-related | 4663 |
| | protein 2-like | |
| P16035_C159 | TIMP2 Metalloproteinase inhibitor 2 | 4664 |
| Q52LW3_C793 | ARHGAP29 Rho GTPase-activating | 4665 |
| | protein 29 | |
| H3BQZ7_C538 | Uncharacterized protein | 4666 |
| Q9BQ90_C213 | KLHDC3 Kelch domain-containing | 4667 |
| | protein 3 | |
| P13797_C167 | PLS3 Plastin-3 | 4668 |
| Q06210_C620 | GFPT1 Glucosamine--fructose-6- | 4669 |
| | phosphate aminotransferase | |
| Q7L576_C98 | CYFIP1 Cytoplasmic FMR1-interacting | 4670 |
| | protein 1 | |
| Q7L576_C1241 | CYFIP1 Cytoplasmic FMRI-interacting | 4671 |
| | protein 1 | |
| Q03001_C5056 | DST Dystonin | 4672 |
| Q03001_C5394 | DST Dystonin | 4673 |
| Q9UFW8_C92 | CGGBP1 CGG triplet repeat-binding | 4674 |
| | protein 1 | |
| P49721_C163 | PSMB2 Proteasome subunit beta type-2 | 4675 |
| Q96I51_C329 | WBSCR16 Williams-Beuren syndrome | 4676 |
| | chromosomal region 16 protein | |
| O60884_C280 | DNAJA2 DnaJ homolog subfamily A | 4677 |
| | member 2 | |
| O60884_C308 | DNAJA2 DnaJ homolog subfamily A | 4678 |
| | member 2 | |
| Q9BV19_C189 | C1orf50 Uncharacterized protein | 4679 |
| | C1orf50 | |
| P29353_C248 | SHC1 SHC-transforming protein 1 | 4680 |
| Q7Z4Q2_C57 | HEATR3 HEAT repeat-containing | 4681 |
| | protein 3 | |
| O75376_C2403 | NCOR1 Nuclear receptor corepressor 1 | 4682 |
| Q9Y6A5_C293 | TACC3 Transforming acidic coiled- | 4683 |
| | coil-containing protein | |
| P31323_C149 | PRKAR2B cAMP-dependent protein | 4684 |
| | kinase type II-beta regulatory subunit | |
| | beta | |
| Q15120_C41 | PDK3 | 4685 |
| P05455_C18 | SSB Lupus La protein | 4686 |
| Q08257_C166 | CRYZ Quinone oxidoreductase | 4687 |
| P54278_C216 | PMS2 Mismatch repair endonuclease | 4688 |
| | PMS2 | |
| P05534_C363 | HLA-A HLA class I histocompatibility | 4689 |
| | antigen, A-24 alpha | |
| Q96P16_C100 | RPRD1A Regulation of nuclear pre- | 4690 |
| | mRNA domain-containing protein | |
| Q9NWK9_C211 | ZNHIT6 Box C/D snoRNA protein 1 | 4691 |
| Q9BZF9_C408 | UACA Uveal autoantigen with coiled- | 4692 |
| | coil domains and ankyrin repeats | |
| P57081_C412 | WDR4 tRNA (guanine-N(7)-)- | 4693 |
| | methyltransferase subunit WDR | |
| Q9H4L7_C861 | SMARCAD1 SWI/SNF-related matrix- | 4694 |
| | associated actin-dependent | |
| Q14149_C671 | MORC3 MORC family CW-type zinc | 4695 |
| | finger protein 3 | |
| Q8WUA7_C151 | TBC1D22A TBC1 domain family | 4696 |
| | member 22A | |
| Q2T9J0_C284 | TYSND1 Peroxisomal leader peptide- | 4697 |
| | processing protease | |
| Q96C86_C37 | DCPS m7GpppX diphosphatase | 4698 |
| P30084_C143 | ECHS1 Enoyl-CoA hydratase, | 4699 |
| | mitochondrial | |
| Q29RF7_C1093 | PDS5A Sister chromatid cohesion | 4700 |
| | protein PDS5 homolog A | |
| O15111_C30 | CHUK Inhibitor of nuclear factor | 4701 |
| | kappa-B kinase subunit | |
| Q9H7B4_C238 | SMYD3 SET and MYND domain- | 4702 |
| | containing protein 3 | |
| Q8TDX7_C53 | NEK7 Serine/threonine-protein kinase | 4703 |
| | Nek7 | |
| Q8TDX7_C298 | NEK7 Serine/threonine-protein kinase | 4704 |
| | Nek7 | |
| Q86W56_C155 | PARG Poly(ADP-ribose) | 4705 |
| | glycohydrolase | |
| P43246_C199 | MSH2 DNA mismatch repair protein | 4706 |
| | Msh2 | |
| P28702_C340 | RXRB Retinoic acid receptor RXR-beta | 4707 |
| Q13283_C73 | G3BP1 Ras GTPase-activating protein- | 4708 |
| | binding protein 1 | |
| O75608_C173 | LYPLA1 Acyl-protein thioesterase 1 | 4709 |
| P23497_C248 | SP100 Nuclear autoantigen Sp-100 | 4710 |
| Q8WX92_C115 | COBRA1 Negative elongation factor B | 4711 |
| O75044_C820 | SRGAP2 SLIT-ROBO Rho GTPase- | 4712 |
| | activating protein 2 | |
| Q13464_C1206 | ROCK1 Rho-associated protein kinase | 4713 |
| | 1 | |
| O96013_C58 | PAK4 Serine/threonine-protein kinase | 4714 |
| | PAK 4 | |
| P15056_C748 | BRAF Serine/threonine-protein kinase | 4715 |
| | B-raf | |
| Q16762_C248 | TST Thiosulfate sulfurtransferase | 4716 |
| P05198_C218 | EIF2S1 Eukaryotic translation initiation | 4717 |
| | factor 2 subunit | |
| P15374_C209 | UCHL3 Ubiquitin carboxyl-terminal | 4718 |
| | hydrolase isozyme L3 | |
| Q9NZN4_C356 | EHD2 EH domain-containing protein 2 | 4719 |
| Q9NZN8_C175 | CNOT2 CCR4-NOT transcription | 4720 |
| | complex subunit 2 | |
| P52294_C529 | KPNA1 Importin subunit alpha-1 | 4721 |
| O76003_C46 | GLRX3 Glutaredoxin-3 | 4722 |
| P35914_C307 | HMGCL Hydroxymethylglutaryl-CoA | 4723 |
| | lyase, mitochondrial | |
| Q00796_C179 | SORD Sorbitol dehydrogenase | 4724 |
| P27797_C105 | CALR Calreticulin | 4725 |
| Q969E4_C44 | TCEAL3 Transcription elongation | 4726 |
| | factor A protein-like 3 | |
| Q7KZF4_C152 | SND1 Staphylococcal nuclease domain- | 4727 |
| | containing protein | |
| Q9P1U0_C78 | ZNRD1 DNA-directed RNA | 4728 |
| | polymerase I subunit RPA12 | |
| Q8WZ82_C152 | OVCA2 Ovarian cancer-associated | 4729 |
| | gene 2 protein | |
| Q9NPD3_{——}C97 | EXOSC4 Exosome complex component | 4730 |
| | RRP41 | |
| P52701_C1294 | MSH6 DNA mismatch repair protein | 4731 |
| | Msh6 | |
| P52701_C1337 | MSH6 DNA mismatch repair protein | 4732 |
| | Msh6 | |
| Q15637_C279 | SF1 Splicing factor 1 | 4733 |
| P49366_C177 | DHPS Deoxyhypusine synthase | 4734 |
| Q96PQ7_C677 | KLHL5 Kelch-like protein 5 | 4735 |
| O95785_C1429 | WIZ Protein Wiz | 4736 |
| P49756_C795 | RBM25 RNA-binding protein 25 | 4737 |
| P49591_C398 | SARS Serine--tRNA ligase, | 4738 |
| | cytoplasmic | |
| P31751_C77 | AKT2 RAC-beta serine/threonine- | 4739 |
| | protein kinase | |
| Q9Y2X3_C205 | NOP58 Nucleolar protein 58 | 4740 |
| Q8IZ21_C568 | PHACTR4 Phosphatase and actin | 4741 |
| | regulator 4 | |
| O00410_C266 | IPO5 Importin-5 | 4742 |
| O95861_C249 | BPNT1 3(2),5-bisphosphate | 4743 |
| | nucleotidase 1 | |
| Q9NTK5_C7S | OLA1 Obg-like ATPase 1 | 4744 |
| P60228_C141 | EIF3E Eukaryotic translation initiation | 4745 |
| | factor 3 subunit | |
| Q05048_C44 | CSTF1 Cleavage stimulation factor | 4746 |
| | subunit 1 | |
| P41229_C1551 | KDM5C Lysine-specific demethylase | 4747 |
| | 5C | |
| Q9NZ09_C161 | UBAP1 Ubiquitin-associated protein 1 | 4748 |
| Q9H4A4_C151 | RNPEP Aminopeptidase B | 4749 |
| Q99704_C308 | DOK1 Docking protein 1 | 4750 |
| Q16531_C128 | DDB1 DNA damage-binding protein 1 | 4751 |
| O43294_C91 | TGFB1I1 Transforming growth factor | 4752 |
| | beta-1-induced transcript 1 | |
| P28482_C65 | MAPK1 Mitogen-activated protein | 4753 |
| | kinase 1 | |
| P36578_C250 | RPL4 60S ribosomal protein L4 | 4754 |
| Q9Y5N6_C88 | ORC6 Origin recognition complex | 4755 |
| | subunit 6 | |
| P62487_C106 | POLR2G DNA-directed RNA | 4756 |
| | polymerase II subunit RPB7 | |
| O00139_C406 | KIF2A Kinesin-like protein KIF2A | 4757 |
| P21266_C119 | GSTM3 Glutathione S-transferase Mu 3 | 4758 |
| O00622_C78 | CYR61 Protein CYR61 | 4759 |
| O00622_C117 | CYR61 Protein CYR61 | 4760 |
| O00622_C353 | CYR61 Protein CYR61 | 4761 |
| Q86WA6_C234 | BPHL Valacyclovir hydrolase | 4762 |
| Q9BUF5_C354 | TUBB6 Tubulin beta-6 chain | 4763 |
| Q07960_C91 | ARHGAP1 Rho GTPase-activating | 4764 |
| | protein 1 | |
| P78346_C257 | RPP30 Ribonuclease P protein subunit | 4765 |
| | p30 | |
| P16885_C849 | PLCG2 1-phosphatidylinositol 4,5- | 4766 |
| | bisphosphate phosphodiese | |
| Q969U7_C168 | PSMG2 Proteasome assembly | 4767 |
| | chaperone 2 | |
| Q9BZG1_C38 | RAB34 Ras-related protein Rab-34 | 4768 |
| Q96KQ7_C596 | EHMT2 Histone-lysine N- | 4769 |
| | methyltransferase EHMT2 | |
| Q8IWB7_C401 | WDFY1 WD repeat and FYVE | 4770 |
| | domain-containing protein 1 | |
| Q14566_C721 | MCM6 DNA replication licensing | 4771 |
| | factor MCM6 | |
| P17844_C170 | DDX5 Probable ATP-dependent RNA | 4772 |
| | helicase DDX5 | |
| O94903_C261 | PROSC Proline synthase co-transcribed | 4773 |
| | bacterial homolog | |
| Q9UPN3_C5346 | MACF1 Microtubule-actin cross- | 4774 |
| | linking factor 1, isoforms | |
| P53634_C355 | CTSC Dipeptidyl peptidase 1 | 4775 |
| Q5VZL5_C948 | ZMYM4 Zinc finger MYM-type | 4776 |
| | protein 4 | |
| A5YKK6_C2359 | CNOT1 CCR4-NOT transcription | 4777 |
| | complex subunit 1 | |
| Q9BWU0_C336 | SLC4A1AP Kanadaptin | 4778 |
| O75369_C1868 | FLNB Filamin-B | 4779 |
| O75369_C1876 | FLNB Filamin-B | 4780 |
| P21980_C620 | TGM2 Protein-glutamine gamma- | 4781 |
| | glutamyltransferase 2 | |
| Q14966_C1279 | ZNF638 Zinc finger protein 638 | 4782 |
| P50395_C335 | GDI2 Rab GDP dissociation inhibitor | 4783 |
| | beta | |
| Q13643_C150 | FHL3 Four and a half LIM domains | 4784 |
| | protein 3 | |
| Q16181_C161 | SEPT7 Septin-7 | 4785 |
| Q9UPR0_C90 | PLCL2 Inactive phospholipase C-like | 4786 |
| | protein 2 | |
| Q9UPR0_C1095 | PLCL2 Inactive phospholipase C-like | 4787 |
| | protein 2 | |
| O14787_C132 | TNPO2 Transportin-2 | 4788 |
| O15067_C1338 | PFAS | 4789 |
| | Phosphoribosylformylglycinamidine | |
| | synthase | |
| Q9NW64_C58 | RBM22 Pre-mRNA-splicing factor | 4790 |
| | RBM22 | |
| O00273_C21 | DFFA DNA fragmentation factor | 4791 |
| | subunit alpha | |
| Q86V21_C493 | AACS Acetoacetyl-CoA synthetase | 4792 |
| Q96GX5_C251 | MASTL Serine/threonine-protein | 4793 |
| | kinase greatwall | |
| Q96GX5_C614 | MASTL Serine/threonine-protein | 4794 |
| | kinase greatwall | |
| O60493_C140 | SNX3 Sorting nexin-3 | 4795 |
| P29279_C120 | CTGF Connective tissue growth factor | 4796 |
| P49790_C585 | NUP153 Nuclear pore complex protein | 4797 |
| | Nup153 | |
| P49790_C753 | NUP153 Nuclear pore complex protein | 4798 |
| | Nup153 | |
| P51114_C99 | FXR1 Fragile X mental retardation | 4799 |
| | syndrome-related protein | |
| Q15170_C88 | TCEAL1 Transcription elongation | 4800 |
| | factor A protein-like 1 | |
| Q8IY18_C229 | SMC5 Structural maintenance of | 4801 |
| | chromosomes protein 5 | |
| P62979_C126 | RPS27A Ubiquitin-40S ribosomal | 4802 |
| | protein S27a | |
| Q9BSJ8_C635 | ESYT1 Extended synaptotagmin-1 | 4803 |
| P35658_C399 | NUP214 Nuclear pore complex protein | 4804 |
| | Nup214 | |
| P27694_C200 | RPA1 Replication protein A 70 kDa | 4805 |
| | DNA-binding subunit | |
| P27694_C503 | RPA1 Replication protein A 70 kDa | 4806 |
| | DNA-binding subunit | |
| P46013_C1843 | MKI67 Antigen KI-67 | 4807 |
| P17858_C212 | PFKL 6-phosphofructokinase, liver | 4808 |
| | type | |
| Q14353_C220 | GAMT Guanidinoacetate N- | 4809 |
| | methyltransferase | |
| Q7Z5J4_C239 | RAI1 Retinoic acid-induced protein 1 | 4810 |
| O75083_C438 | WDR1 WD repeat-containing protein 1 | 4811 |
| Q8WXH0_C553 | SYNE2 Nesprin-2 | 4812 |
| Q14137_C469 | BOP1 Ribosome biogenesis protein | 4813 |
| | BOP1 | |
| Q8N4X5_C781 | AFAP1L2 Actin filament-associated | 4814 |
| | protein 1-like 2 | |
| P52732_C773 | KIF11 Kinesin-like protein KIF11 | 4815 |
| Q7Z6Z7_C1879 | HUWE1 E3 ubiquitin-protein ligase | 4816 |
| | HUWE1 | |
| Q7Z6Z7_C4099 | HUWE1 E3 ubiquitin-protein ligase | 4817 |
| | HUWE1 | |
| P11216_C496 | PYGB Glycogen phosphorylase, brain | 4818 |
| | form | |
| Q16514_C143 | TAF12 Transcription initiation factor | 4819 |
| | TFIID subunit 12 | |
| Q9H3P2_C471 | WHSC2 Negative elongation factor A | 4820 |
| Q9UFC0_C88 | LRWD1 Leucine-rich repeat and WD | 4821 |
| | repeat-containing protein | |
| Q9UFC0_C249 | LRWD1 Leucine-rich repeat and WD | 4822 |
| | repeat-containing prote | |
| Q96RR4_C223 | CAMKK2 Calcium/calmodulin- | 4823 |
| | dependent protein kinase kinase | |
| Q9UJU6_C97 | DBNL Drebrin-like protein | 4824 |
| Q9NYZ3_C45 | GTSE1 G2 and S phase-expressed | 4825 |
| | protein 1 | |
| Q9ULI3_C1360 | HEG1 Protein HEG homolog 1 | 4826 |
| P38606_C277 | ATP6V1A V-type proton ATPase | 4827 |
| | catalytic subunit A | |
| Q9UPQ0_C333 | LIMCH1 LIM and calponin homology | 4828 |
| | domains-containing protein | |
| Q7L2H7_C125 | EIF3M Eukaryotic translation initiation | 4829 |
| | factor 3 subunit | |
| Q9BPX3_C177 | NCAPG Condensin complex subunit 3 | 4830 |
| O95757_C540 | HSPA4L Heat shock 70 kDa protein 4L | 4831 |
| O95757_C589 | HSPA4L Heat shock 70 kDa protein 4L | 4832 |
| Q9BWH6_C195 | RPAP1 RNA polymerase Il-associated | 4833 |
| | protein 1 | |
| Q8NHV4_C314 | NEDD1 Protein NEDD1 | 4834 |
| Q9UJV9_C568 | DDX41 Probable ATP-dependent RNA | 4835 |
| | helicase DDX41 | |
| P13010_C157 | XRCC5 X-ray repair cross- | 4836 |
| | complementing protein 5 | |
| O95340_C202 | PAPSS2 Bifunctional 3- | 4837 |
| | phosphoadenosine 5-phosphosulfate | |
| Q5JZ73_C9 | LINC00207 Protein LINC00207 | 4838 |
| Q6DN90_C214 | IQSEC1 IQ motif and SEC7 domain- | 4839 |
| | containing protein 1 | |
| Q9BZE9_C127 | ASPSCR1 Tether containing UBX | 4840 |
| | domain for GLUT4 | |
| O95294_C386 | RASAL1 RasGAP-activating-like | 4841 |
| | protein 1 | |
| O95294_C534 | RASAL1 RasGAP-activating-like | 4842 |
| | protein 1 | |
| P06737_C496 | PYGL Glycogen phosphorylase, liver | 4843 |
| | form | |
| Q92616_C1482 | GCN1L1 Translational activator GCN1 | 4844 |
| Q9Y6I9_C165 | TEX264 Testis-expressed sequence 264 | 4845 |
| | protein | |
| Q99996_C1966 | AKAP9 A-kinase anchor protein 9 | 4846 |
| Q6UB99_C2640 | ANKRD11 Ankyrin repeat domain- | 4847 |
| | containing protein 11 | |
| Q9NRV9_C168 | HEBP1 Heme-binding protein 1 | 4848 |
| Q6ZUT6_C437 | C15orf52 Uncharacterized protein | 4849 |
| | C15orf52 | |
| P15407_C172 | FOSL1 Fos-related antigen 1 | 4850 |
| Q9BYG3_C237 | MKI67IP MKI67 FHA domain- | 4851 |
| | interacting nucleolar phosphoprotein | |
| P49327_C496 | FASN Fatty acid synthase | 4852 |
| Q99436_C74 | PSMB7 Proteasome subunit beta type-7 | 4853 |
| Q96BR1_C308 | SGK3 Serine/threonine-protein kinase | 4854 |
| | Sgk3 | |
| Q9GZS1_C200 | POLR1E DNA-directed RNA | 4855 |
| | polymerase I subunit RPA49 | |
| P41091_C269 | EIF2S3 Eukaryotic translation initiation | 4856 |
| | factor 2 subunit | |
| Q12769_C659 | NUP160 Nuclear pore complex protein | 4857 |
| | Nup160 | |
| P13489_C313 | RNH1 Ribonuclease inhibitor | 4858 |
| Q9H5N1_C482 | RABEP2 Rab GTPase-binding effector | 4859 |
| | protein 2 | |
| Q9Y2Z0_C49 | SUGT1 Suppressor of G2 allele of | 4860 |
| | SKP1 homolog | |
| P48444_C441 | ARCN1 Coatomer subunit delta | 4861 |
| Q12888_C1375 | TP53BP1 Tumor suppressor p53- | 4862 |
| | binding protein 1 | |
| P14921_C112 | ETS1 Protein C-ets-1 | 4863 |
| Q5JTZ9_C443 | AARS2 Alanine--tRNA ligase, | 4864 |
| | mitochondrial | |
| Q92990_C406 | GLMN Glomulin | 4865 |
| Q9P0J1_C132 | PDP1 | 4866 |
| Q8NBJ5_C369 | GLT25D1 Procollagen | 4867 |
| | galactosyltransferase 1 | |
| P01591_C94 | IGJ Immunoglobulin J chain | 4868 |
| Q96QR8_C238 | PURB Transcriptional activator protein | 4869 |
| | Pur-beta | |
| Q99832_C326 | CCT7 T-complex protein 1 subunit eta | 4870 |
| O95218_C71 | ZRANB2 Zinc finger Ran-binding | 4871 |
| | domain-containing protein | |
| O43237_C191 | DYNC1LI2 Cytoplasmic dynein 1 light | 4872 |
| | intermediate chain 2 | |
| Q92576_C276 | PHF3 PHD finger protein 3 | 4873 |
| Q9HAU0_C473 | PLEKHA5 Pleckstrin homology | 4874 |
| | domain-containing family A member | |
| O95425_C76 | SVIL Supervillin | 4875 |
| Q8TD30_C158 | GPT2 Alanine aminotransferase 2 | 4876 |
| Q8TD30_C347 | GPT2 Alanine aminotransferase 2 | 4877 |
| Q14790_C409 | CASP8 Caspase-8 | 4878 |
| P09211_C170 | GSTP1 Glutathione S-transferase P | 4879 |
| Q03519_C641 | TAP2 Antigen peptide transporter 2 | 4880 |
| Q9ULW0_C462 | TPX2 Targeting protein for Xklp2 | 4881 |
| Q16222_C251 | UAP1 UDP-N-acetylhexosamine | 4882 |
| | pyrophosphorylase | |
| P61158_C408 | ACTR3 Actin-related protein 3 | 4883 |
| Q6WKZ4_C318 | RAB11FIP1 Rab11 family-interacting | 4884 |
| | protein 1 | |
| Q9HCE7_C725 | SMURF1 E3 ubiquitin-protein ligase | 4885 |
| | SMURF1 | |
| Q13564_C153 | NAE1 NEDD8-activating enzyme E1 | 4886 |
| | regulatory subunit | |
| Q13564_C384 | NAE1 NEDD8-activating enzyme E1 | 4887 |
| | regulatory subunit | |
| P31146_C24 | CORO1A Coronin-1A | 4888 |
| Q96JM7_C169 | L3MBTL3 Lethal(3)malignant brain | 4889 |
| | tumor-like protein 3 | |
| P07199_C135 | CENPB Major centromere autoantigen | 4890 |
| | B | |
| O00442_C28 | RTCA RNA 3-terminal phosphate | 4891 |
| | cyclase | |
| P13639_C388 | EEF2 Elongation factor 2 | 4892 |
| Q9P0K7_C468 | RAI14 Ankycorbin | 4893 |
| P35606_C276 | COPB2 Coatomer subunit beta | 4894 |
| P46199_C290 | MTIF2 Translation initiation factor IF- | 4895 |
| | 2, mitochondrial | |
| P78527_C232 | PRKDC DNA-dependent protein kinase | 4896 |
| | catalytic subunit | |
| P78527_C1455 | PRKDC DNA-dependent protein kinase | 4897 |
| | catalytic subunit | |
| P78527_C4045 | PRKDC DNA-dependent protein kinase | 4898 |
| | catalytic subunit | |
| P11908_C60 | PRPS2 Ribose-phosphate | 4899 |
| | pyrophosphokinase 2 | |
| Q9H410_C65 | DSN1 Kinetochore-associated protein | 4900 |
| | DSN1 homolog | |
| Q14498_C478 | RBM39 RNA-binding protein 39 | 4901 |
| O75694_C1344 | NUP155 Nuclear pore complex protein | 4902 |
| | Nup155 | |
| Q8WU76_C79 | SCFD2 Sec1 family domain-containing | 4903 |
| | protein 2 | |
| Q8IU81_C207 | IRF2BP1 Interferon regulatory factor 2- | 4904 |
| | binding protein 1 | |
| P49189_C443 | ALDH9A1 4- | 4905 |
| | trimethylaminobutyraldehyde | |
| | dehydrogenase | |
| Q14789_C1257 | GOLGB1 Golgin subfamily B member | 4906 |
| | 1 | |
| Q8N5K1_C92 | CISD2 CDGSH iron-sulfur domain- | 4907 |
| | containing protein 2 | |
| O43592_C693 | XPOT Exportin-T | 4908 |
| O43374_C302 | RASA4 Ras GTPase-activating protein | 4909 |
| | 4 | |
| O43374_C657 | RASA4 Ras GTPase-activating protein | 4910 |
| | 4 | |
| Q9NUQ3_C480 | TXLNG Gamma-taxilin | 4911 |
| P63104_C189 | YWHAZ 14-3-3 protein zeta/delta | 4912 |
| O60216_C392 | RAD21 Double-strand-break repair | 4913 |
| | protein rad21 homolog | |
| Q8N3U4_C869 | STAG2 Cohesin subunit SA-2 | 4914 |
| Q9ULV4_C283 | CORO1C Coronin-1C | 4915 |
| P61160_C221 | ACTR2 Actin-related protein 2 | 4916 |
| Q12904_C161 | AIMP1 Aminoacyl tRNA synthase | 4917 |
| | complex-interacting multifunctional | |
| | protein 1 | |
| Q9H9P8_C187 | L2HGDH L-2-hydroxyglutarate | 4918 |
| | dehydrogenase, mitochondrial | |
| Q7Z2W4_C272 | ZC3HAV1 Zinc finger CCCH-type | 4919 |
| | antiviral protein 1 | |
| P30153_C148 | PPP2R1A Serine/threonine-protein | 4920 |
| | phosphatase 2A 65 kDa regulatory | |
| | subunit A alpha isoform | |
| Q92797_C969 | SYMPK Symplekin | 4921 |
| Q9Y3B7_C50 | MRPL11 39S ribosomal protein L11, | 4922 |
| | mitochondrial | |
| Q9BV44_C391 | THUMPD3 THUMP domain- | 4923 |
| | containing protein 3 | |
| O14578_C402 | CIT Citron Rho-interacting kinase | 4924 |
| Q9NVU0_C70 | POLR3E DNA-directed RNA | 4925 |
| | polymerase III subunit RPC5 | |
| Q92947_C115 | GCDH Glutaryl-CoA dehydrogenase, | 4926 |
| | mitochondrial | |
| Q6PJ69_C320 | TRIM65 Tripartite motif-containing | 4927 |
| | protein 65 | |
| Q15027_C501 | ACAP1 Arf-GAP with coiled-coil, | 4928 |
| | ANK repeat and PH domain | |
| Q15020_C341 | SART3 Squamous cell carcinoma | 4929 |
| | antigen recognized by T-cells 3 | |
| Q9Y6J9_C41 | TAF6L TAF6-like RNA polymerase II | 4930 |
| | p300/CBP-associated factor 6 like | |
| P17812_C216 | CTPS1 CTP synthase 1 | 4931 |
| Q96GG9_C29 | DCUN1D1 DCN1-like protein 1 | 4932 |
| Q14315_C1448 | FLNC Filamin-C | 4933 |
| Q9NSP4_C40 | CENPM Centromere protein M | 4934 |
| Q9Y3B2_C40 | EXOSC1 Exosome complex component | 4935 |
| | CSL4 | |
| Q9H4K7_C206 | GTPBP5 GTP-binding protein 5 | 4936 |
| P62140_C201 | PPP1CB Serine/threonine-protein | 4937 |
| | phosphatase PP1-beta catalytic subunit | |
| Q9H063_C133 | MAF1 Repressor of RNA polymerase | 4938 |
| | III transcription MAF1 | |
| P23434_C138 | GCSH Glycine cleavage system H | 4939 |
| | protein, mitochondrial | |
| Q5JPH6_C386 | EARS2 Probable glutamate--tRNA | 4940 |
| | ligase, mitochondrial | |
| O14976_C1142 | GAK Cyclin-G-associated kinase | 4941 |
| O14974_C553 | PPP1R12A Protein phosphatase 1 | 4942 |
| | regulatory subunit 12A | |
| Q9H9A6_C438 | LRRC40 Leucine-rich repeat- | 4943 |
| | containing protein 40 | |
| Q13315_C2607 | ATM Serine-protein kinase ATM | 4944 |
| P51116_C270 | FXR2 Fragile X mental retardation | 4945 |
| | syndrome-related protein | |
| Q8N3X1_C877 | FNBP4 Formin-binding protein 4 | 4946 |
| P36404_C153 | ARL2 ADP-ribosylation factor-like | 4947 |
| | protein 2 | |
| Q9HD64_C43 | XAGE1E G antigen family D member | 4948 |
| | 2 | |
| Q8NF50_C1230 | DOCK8 Dedicator of cytokinesis | 4949 |
| | protein 8 | |
| Q8WWM7_C628 | ATXN2L Ataxin-2-like protein | 4950 |
| P55795_C290 | HNRNPH2 Heterogeneous nuclear | 4951 |
| | ribonucleoprotein H2 | |
| Q9BYW2_C1471 | SETD2 Histone-lysine N- | 4952 |
| | methyltransferase SETD2 | |
| O15143_C202 | ARPC1B Actin-related protein 2/3 | 4953 |
| | complex subunit 1B | |
| O15294_C323 | OGT UDP-N-acetylglucosamine-- | 4954 |
| | peptide N-acetylglucosamine (GlcNAc) | |
| | transferase | |
| Q92945_C176 | KHSRP Far upstream element-binding | 4955 |
| | protein 2 | |
| O43847_C60 | NRD1 Nardilysin | 4956 |
| Q9H9A5_C504 | CNOT10 CCR4-NOT transcription | 4957 |
| | complex subunit 10 | |
| Q9H9A5_C633 | CNOT10 CCR4-NOT transcription | 4958 |
| | complex subunit 10 | |
| P10155_C71 | TROVE2 60 kDa SS-A/Ro | 4959 |
| | ribonucleoprotein | |
| P52789_C386 | HK2 Hexokinase-2 | 4960 |
| Q01469_C43 | FABP5 Fatty acid-binding protein, | 4961 |
| | epidermal | |
| P46734_C305 | MAP2K3 Dual specificity mitogen- | 4962 |
| | activated protein kinase | |
| P35579_C511 | MYH9 Myosin-9 | 4963 |
| P35579_C789 | MYH9 Myosin-9 | 4964 |
| Q9Y3C1_C36 | NOP16 Nucleolar protein 16 | 4965 |
| P62888_C52 | RPL30 60S ribosomal protein L30 | 4966 |
| Q8TAQ2_C495 | SMARCC2 SWI/SNF complex subunit | 4967 |
| | SMARCC2 | |
| O94854_C242 | KIAA0754 Uncharacterized protein | 4968 |
| | KIAA0754 | |
| P23526_C113 | AHCY Adenosylhomocysteinase | 4969 |
| Q6QNY1_C41 | BLOC1S2 Biogenesis of lysosome- | 4970 |
| | related organelles complex | |
| Q7Z7A4_C196 | PXK PX domain-containing protein | 4971 |
| | kinase-like protein | |
| Q14699_C128 | RFTN1 Raftlin | 4972 |
| Q14694_C254 | USP10 Ubiquitin carboxyl-terminal | 4973 |
| | hydrolase 10 | |
| Q13435_C661 | SF3B2 Splicing factor 3B subunit 2 | 4974 |
| P22692_C38 | IGFBP4 Insulin-like growth factor- | 4975 |
| | binding protein 4 | |
| P22692_C215 | IGFBP4 Insulin-like growth factor- | 4976 |
| | binding protein 4 | |
| P60900_C161 | PSMA6 Proteasome subunit alpha type- | 4977 |
| | 6 | |
| Q9NWY4_C17 | C4orf27 UPF0609 protein C4orf27 | 4978 |
| P12004_C62 | PCNA Proliferating cell nuclear antigen | 4979 |
| O00148_C238 | DDX39A ATP-dependent RNA | 4980 |
| | helicase DDX39A | |
| Q9BW19_C144 | KIFC1 Kinesin-like protein KIFC1 | 4981 |
| Q9BW19_C557 | KIFC1 Kinesin-like protein KIFC1 | 4982 |
| Q15477_C247 | SKIV2L Helicase SKI2W | 4983 |
| Q9NQ89_C9 | C12orf4 Uncharacterized protein | 4984 |
| | C12orf4 | |
| Q9Y2R9_C152 | MRPS7 28S ribosomal protein S7, | 4985 |
| | mitochondrial | |
| Q12933_C287 | TRAF2 TNF receptor-associated factor | 4986 |
| | 2 | |
| Q12931_C501 | TRAP1 Heat shock protein 75 kDa, | 4987 |
| | mitochondrial | |
| Q9NQS1_C251 | AVEN Cell death regulator Aven | 4988 |
| Q9UBB4_C134 | ATXN10 Ataxin-10 | 4989 |
| Q9UBB4_C382 | ATXN10 Ataxin-10 | 4990 |
| Q9C0C9_C910 | UBE2O Ubiquitin-conjugating enzyme | 4991 |
| | E2 O | |
| Q9BTW9_C234 | TBCD Tubulin-specific chaperone D | 4992 |
| Q9Y520_C2340 | PRRC2C Protein PRRC2C | 4993 |
| O75533_C933 | SF3B1 Splicing factor 3B subunit 1 | 4994 |
| O75533_C1059 | SF3B1 Splicing factor 3B subunit 1 | 4995 |
| Q96BD8_C29 | SKA1 Spindle and kinetochore- | 4996 |
| | associated protein 1 | |
| Q14192_C28 | FHL2 Four and a half LIM domains | 4997 |
| | protein 2 | |
| Q14192_C129 | FHL2 Four and a half LIM domains | 4998 |
| | protein 2 | |
| Q92844_C328 | TANK TRAF family member- | 4999 |
| | associated NF-kappa-B activator | |
| Q9Y4H4_C116 | GPSM3 G-protein-signaling modulator | 5000 |
| | 3 | |
| P30154_C389 | PPP2R1B Serine/threonine-protein | 5001 |
| | phosphatase 2A 65 kDa regulatory | |
| | subunit A beta | |
| Q9UN37_C403 | VPS4A Vacuolar protein sorting- | 5002 |
| | associated protein 4A | |
| Q96RL1_C121 | UIMC1 BRCA1-A complex subunit | 5003 |
| | RAP80 | |
| Q8WZA9_C443 | IRGQ Immunity-related GTPase family | 5004 |
| | Q protein | |
| Q8IZT6_C51 | ASPM Abnormal spindle-like | 5005 |
| | microcephaly-associated protein | |
| P11586_C408 | MTHFD1 C-1-tetrahydrofolate | 5006 |
| | synthase, cytoplasmic | |
| P11586_C691 | MTHFD1 C-1-tetrahydrofolate | 5007 |
| | synthase, cytoplasmic | |
| P11586_C785 | MTHFD1 C-1-tetrahydrofolate | 5008 |
| | synthase, cytoplasmic | |
| P21912_C243 | SDHB Succinate dehydrogenase | 5009 |
| Q99471_C49 | PFDN5 Prefoldin subunit 5 | 5010 |
| P48735_C336 | IDH2 Isocitrate dehydrogenase | 5011 |
| Q9UJM3_C430 | ERRFI1 ERBB receptor feedback | 5012 |
| | inhibitor 1 | |
| Q6IA69_C627 | NADSYN1 Glutamine-dependent | 5013 |
| | NAD(+) synthetase | |
| P61221_C201 | ABCE1 ATP-binding cassette sub- | 5014 |
| | family E member 1 | |
| Q7L1V2_C326 | MON1B Vacuolar fusion protein | 5015 |
| | MON1 homolog B | |
| Q7L1V2_C349 | MON1B Vacuolar fusion protein | 5016 |
| | MON1 homolog B | |
| P62136_C202 | PPP1CA Serine/threonine-protein | 5017 |
| | phosphatase PP1-alpha catalytic subunit | |
| Q8IWS0_C87 | PHF6 PHD finger protein 6 | 5018 |
| O95239_C1159 | KIF4A Chromosome-associated kinesin | 5019 |
| | KIF4A | |
| P05091_C66 | ALDH2 Aldehyde dehydrogenase, | 5020 |
| | mitochondrial | |
| O75817_C66 | POP7 Ribonuclease P protein subunit | 5021 |
| | p20 | |
| O15231_C650 | ZNF185 Zinc finger protein 185 | 5022 |
| Q92922_C520 | SMARCC1 SWI/SNF complex subunit | 5023 |
| | SMARCC1 | |
| Q9NSY1_C32 | BMP2K BMP-2-inducible protein | 5024 |
| | kinase | |
| O60671_C148 | RAD1 Cell cycle checkpoint protein | 5025 |
| | RAD1 | |
| O75676_C425 | RPS6KA4 Ribosomal protein S6 kinase | 5026 |
| | alpha-4 | |
| Q01082_C964 | SPTBN1 Spectrin beta chain, non- | 5027 |
| | erythrocytic 1 | |
| P61812_C380 | TGFB2 Transforming growth factor | 5028 |
| | beta-2 | |
| P42704_C361 | LRPPRC Leucine-rich PPR motif- | 5029 |
| | containing protein, mitochondrial | |
| P42858_C777 | HTT Huntingtin | 5030 |
| P42858_C1808 | HTT Huntingtin | 5031 |
| P34932_C146 | HSPA4 Heat shock 70 kDa protein 4 | 5032 |
| P34932_C167 | HSPA4 Heat shock 70 kDa protein 4 | 5033 |
| O43447_C174 | PPIH Peptidyl-prolyl cis-trans | 5034 |
| | isomerase H | |
| P31937_C75 | HIBADH 3-hydroxyisobutyrate | 5035 |
| | dehydrogenase, mitochondrial | |
| P31937_C199 | HIBADH 3-hydroxyisobutyrate | 5036 |
| | dehydrogenase, mitochondrial | |
| Q8IXK0_C740 | PHC2 Polyhomeotic-like protein 2 | 5037 |
| Q9NUI1_C120 | DECR2 Peroxisomal 2,4-dienoyl-CoA | 5038 |
| | reductase | |
| Q9NR50_C334 | EEF2B3 Translation initiation factor | 5039 |
| | eIF-2B subunit gamma | |
| Q9NV56_C59 | MRGBP MRG-binding protein | 5040 |
| Q9BYV9_C340 | BACH2 Transcription regulator protein | 5041 |
| | BACH2 | |
| P08237_C351 | PFKM 6-phosphofructokinase, muscle | 5042 |
| | type | |
| Q15118_C240 | PDK1 | 5043 |
| Q5M775_C805 | SPECC1 Cytospin-B | 5044 |
| Q9H845_C507 | ACAD9 Acyl-CoA dehydrogenase | 5045 |
| | family member 9, mitochondrial | |
| Q8TF72_C1983 | SHROOM3 Protein Shroom3 | 5046 |
| Q7Z5L9_C521 | IRF2BP2 Interferon regulatory factor 2- | 5047 |
| | binding protein 2 | |
| Q86VP6_C237 | CAND1 Cullin-associated NEDD8- | 5048 |
| | dissociated protein 1 | |
| O43148_C95 | RNMT mRNA cap guanine-N7 | 5049 |
| | methyltransferase | |
| P27144_C104 | AK4 Adenylate kinase isoenzyme 4, | 5050 |
| | mitochondrial | |
| P67775_C269 | PPP2CA Serine/threonine-protein | 5051 |
| | phosphatase 2A catalytic | |
| Q92667_C438 | AKAP1 A-kinase anchor protein 1, | 5052 |
| | mitochondrial | |
| Q9NYL2_C285 | MLTK Mitogen-activated protein | 5053 |
| | kinase kinase kinase MLT | |
| Q9Y5X2_C455 | SNX8 Sorting nexin-8 | 5054 |
| Q14152_C185 | EIF3A Eukaryotic translation initiation | 5055 |
| | factor 3 subunit | |
| Q15669_C108 | RHOH Rho-related GTP-binding | 5056 |
| | protein RhoH | |
| P19623_C89 | SRM Spermidine synthase | 5057 |
| P63244_C153 | GNB2L1 Guanine nucleotide-binding | 5058 |
| | protein subunit beta-2-like 1 | |
| Q9NZB2_C892 | FAM120A Constitutive coactivator of | 5059 |
| | PPAR-gamma-like protein | |
| Q15398_C696 | DLGAP5 Disks large-associated protein | 5060 |
| | 5 | |
| O60566_C51 | BUB1B Mitotic checkpoint | 5061 |
| | serine/threonine-protein kinase | |
| Q9UDY4_C175 | DNAJB4 DnaJ homolog subfamily B | 5062 |
| | member 4 | |
| Q03001_C6799 | DST Dystonin | 5063 |
| P11182_C279 | DBT Lipoamide acyltransferase | 5064 |
| | component of branched-chain | |
| | transacylase E2 | |
| P56537_C110 | EIF6 Eukaryotic translation initiation | 5065 |
| | factor 6 | |
| O15037_C157 | KHNYN Protein KHNYN | 5066 |
| Q15052_C57 | ARHGEF6 Rho guanine nucleotide | 5067 |
| | exchange factor 6 | |
| Q15052_C553 | ARHGEF6 Rho guanine nucleotide | 5068 |
| | exchange factor 6 | |
| O00592_C381 | PODXL Podocalyxin | 5069 |
| E7EVH7_C408 | KLC1 Kinesin light chain 1 | 5070 |
| E7EVH7_C628 | KLC1 Kinesin light chain 1 | 5071 |
| Q9H2U2_C161 | PPA2 Inorganic pyrophosphatase 2, | 5072 |
| | mitochondrial | |
| P24390_C29 | KDELR1 ER lumen protein retaining | 5073 |
| | receptor 1 | |
| Q9H0W9_C149 | C11orf54 Ester hydrolase C11orf54 | 5074 |
| Q9H0W9_C154 | C11orf54 Ester hydrolase C11orf54 | 5075 |
| Q9UK59_C339 | DBR1 Lariat debranching enzyme | 5076 |
| Q6P2Q9_C435 | PRPF8 Pre-mRNA-processing-splicing | 5077 |
| | factor 8 | |
| O75586_C137 | MED6 Mediator of RNA polymerase II | 5078 |
| | transcription subunit | |
| Q99933_C213 | BAG1 BAG family molecular | 5079 |
| | chaperone regulator 1 | |
| Q9H4L5_C515 | OSBPL3 Oxysterol-binding protein- | 5080 |
| | related protein 3 | |
| Q14149_C694 | MORC3 MORC family CW-type zinc | 5081 |
| | finger protein 3 | |
| Q92546_C314 | RGP1 Retrograde Golgi transport | 5082 |
| | protein RGP1 homolog | |
| P46821_C1228 | MAP1B Microtubule-associated protein | 5083 |
| | 1B | |
| O60343_C753 | TBC1D4 TBC1 domain family member | 5084 |
| | 4 | |
| Q9UBT2_C432 | UBA2 SUMO-activating enzyme | 5085 |
| | subunit 2 | |
| Q86SQ0_C935 | PHLDB2 Pleckstrin homology-like | 5086 |
| | domain family B member 2 | |
| Q96A65_C957 | EXOC4 Exocyst complex component 4 | 5087 |
| O14980_C498 | XPO1 Exportin-1 | 5088 |
| Q9ULP9_C29 | TBC1D24 TBC1 domain family | 5089 |
| | member 24 | |
| P28290_C448 | SSFA2 Sperm-specific antigen 2 | 5090 |
| P31948_C62 | STIP1 Stress-induced-phosphoprotein 1 | 5091 |
| O95071_C739 | UBR5 E3 ubiquitin-protein ligase | 5092 |
| | UBR5 | |
| Q96L91_C694 | EP400 E1A-binding protein p400 | 5093 |
| Q9Y2L1_C357 | DIS3 Exosome complex exonuclease | 5094 |
| | RRP44 | |
| Q96L94_C184 | SNX22 Sorting nexin-22 | 5095 |
| Q15437_C40 | SEC23B Protein transport protein | 5096 |
| | Sec23B | |
| Q9H0F6_C140 | SHARPIN Sharpin | 5097 |
| Q6NZY4_C633 | ZCCHC8 Zinc finger CCHC domain- | 5098 |
| | containing protein 8 | |
| Q6NVY1_C271 | HIBCH 3-hydroxyisobutyryl-CoA | 5099 |
| | hydrolase, mitochondrial | |
| P38432_C425 | COIL Coilin | 5100 |
| Q29RF7_C508 | PDS5A Sister chromatid cohesion | 5101 |
| | protein PDS5 homolog A | |
| Q29RF7_C925 | PDS5A Sister chromatid cohesion | 5102 |
| | protein PDS5 homolog A | |
| Q8N5C7_C40 | DTWD1 DTW domain-containing | 5103 |
| | protein 1 | |
| O60437_C660 | PPL Periplakin | 5104 |
| Q15042_C678 | RAB3GAP1 Rab3 GTPase-activating | 5105 |
| | protein catalytic subunit | |
| O15111_C406 | CHUK Inhibitor of nuclear factor | 5106 |
| | kappa-B kinase subunit | |
| P49915_C554 | GMPS GMP synthase | 5107 |
| Q9Y490_C709 | TLN1 Talin-1 | 5108 |
| Q9Y490_C1661 | TLN1 Talin-1 | 5109 |
| Q9UGI8_C238 | TES Testin | 5110 |
| Q9Y3D3_C26 | MRPS16 28S ribosomal protein S16, | 5111 |
| | mitochondrial | |
| Q8WUY1_C104 | THEM6 UPF0670 protein THEM6 | 5112 |
| Q969Y2_C489 | GTPBP3 tRNA modification GTPase | 5113 |
| | GTPBP3, mitochondrial | |
| Q13042_C194 | CDC16 Cell division cycle protein 16 | 5114 |
| | homolog | |
| Q8IWP9_C108 | CCDC28A Coiled-coil domain- | 5115 |
| | containing protein 28A | |
| Q13469_C386 | NFATC2 Nuclear factor of activated T- | 5116 |
| | cells, cytoplasmic 2 | |
| Q13263_C232 | TRIM28 Transcription intermediary | 5117 |
| | factor 1-beta | |
| Q16822_C210 | PCK2 Phosphoenolpyruvate | 5118 |
| | carboxykinase | |
| Q96BY7_C891 | ATG2B Autophagy-related protein 2 | 5119 |
| | homolog B | |
| Q9H8M7_C27 | FAM188A Protein FAM188A | 5120 |
| Q8N7H5_C36 | PAF1 RNA polymerase II-associated | 5121 |
| | factor 1 homolog | |
| O00116_C226 | AGPS | 5122 |
| | Alkyldihydroxyacetonephosphate | |
| | synthase, peroxisom | |
| O14641_C354 | DVL2 Segment polarity protein | 5123 |
| | dishevelled homolog DVL-2 | |
| P49588_C723 | AARS Alanine--tRNA ligase, | 5124 |
| | cytoplasmic | |
| Q9H0G5_C171 | NSRP1 Nuclear speckle splicing | 5125 |
| | regulatory protein 1 | |
| Q9NZN5_C784 | ARIIGEF12 Rho guanine nucleotide | 5126 |
| | exchange factor 12 | |
| Q5JVF3_C362 | PCID2 PCI domain-containing protein | 5127 |
| | 2 | |
| P56192_C566 | MARS Methionine--tRNA ligase, | 5128 |
| | cytoplasmic | |
| P07954_C434 | FH Fumarate hydratase, mitochondrial | 5129 |
| P09429_C106 | HMGB1 High mobility group protein | 5130 |
| | B1 | |
| Q9BVP2_C158 | GNL3 Guanine nucleotide-binding | 5131 |
| | protein-like 3 | |
| Q13813_C315 | SPTAN1 Spectrin alpha chain, non- | 5132 |
| | erythrocytic 1 | |
| Q66PJ3_C159 | ARL6IP4 ADP-ribosylation factor-like | 5133 |
| | protein 6-interacting | |
| P55957_C15 | BID BH3-interacting domain death | 5134 |
| | agonist | |
| Q9H4A3_C547 | WNK1 Serine/threonine-protein kinase | 5135 |
| | WNK1 | |
| P27797_C137 | CALR Calreticulin | 5136 |
| P15498_C83 | VAV1 Proto-oncogene vav | 5137 |
| Q9Y572_C365 | RIPK3 Receptor-interacting | 5138 |
| | serine/threonine-protein kina | |
| Q8IX90_C290 | SKA3 Spindle and kinetochore- | 5139 |
| | associated protein 3 | |
| O96019_C32 | ACTL6A Actin-like protein 6A | 5140 |
| P46939_C447 | UTRN Utrophin | 5141 |
| P46939_C1759 | UTRN Utrophin | 5142 |
| P52701_C1275 | MSH6 DNA mismatch repair protein | 5143 |
| | Msh6 | |
| Q14160_C1612 | SCRIB Protein scribble homolog | 5144 |
| O14879_C39 | IFIT3 Interferon-induced protein with | 5145 |
| | tetratricopeptide | |
| Q16543_C64 | CDC37 Hsp90 co-chaperone Cdc37 | 5146 |
| O75962_C868 | TRIO Triple functional domain protein | 5147 |
| Q9H3S7_C1466 | PTPN23 Tyrosine-protein phosphatase | 5148 |
| | non-receptor type 23 | |
| P26196_C102 | DDX6 Probable ATP-dependent RNA | 5149 |
| | helicase DDX6 | |
| P33992_C207 | MCM5 DNA replication licensing | 5150 |
| | factor MCM5 | |
| O14893_C34 | GEMIN2 Gem-associated protein 2 | 5151 |
| Q86SE5_C51 | RALYL RNA-binding Raly-like protein | 5152 |
| Q9BUN5_C13 | CCDC28B Coiled-coil domain- | 5153 |
| | containing protein 28B | |
| Q9NR12_C388 | PDLIM7 PDZ and LIM domain protein | 5154 |
| | 7 | |
| Q9NR19_C75 | ACSS2 Acetyl-coenzyme A synthetase, | 5155 |
| | cytoplasmic | |
| Q9Y2X0_C790 | MED16 Mediator of RNA polymerase | 5156 |
| | II transcription subunit | |
| Q9Y2X0_C801 | MED16 Mediator of RNA polymerase | 5157 |
| | II transcription subunit | |
| Q00325_C237 | SLC25A3 Phosphate carrier protein, | 5158 |
| | mitochondrial | |
| O95861_C243 | BPNT1 3(2),5-bisphosphate | 5159 |
| | nucleotidase 1 | |
| Q5VUA4_C475 | ZNF318 Zinc finger protein 318 | 5160 |
| Q8TDZ2_C837 | MICAL1 Protein-methionine sulfoxide | 5161 |
| | oxidase MICAL1 | |
| Q7RTV0_C61 | PHF5A PHD finger-like domain- | 5162 |
| | containing protein 5A | |
| Q9Y4R8_C644 | TELO2 Telomere length regulation | 5163 |
| | protein TEL2 homolog | |
| Q2PPJ7_C838 | RALGAPA2 Ral GTPase-activating | 5164 |
| | protein subunit alpha-2 | |
| P09382_C131 | LGALS1 Galectin-1 | 5165 |
| O95081_C30 | AGFG2 Arf-GAP domain and FG | 5166 |
| | repeat-containing protein 2 | |
| Q8WV74_C224 | NUDT8 Nucleoside diphosphate-linked | 5167 |
| | moiety X motif 8, mitochondrial | |
| Q86Y37_C166 | CACUL1 CDK2-associated and cullin | 5168 |
| | domain-containing prote | |
| O43414_C258 | ERI3 ERI1 exoribonuclease 3 | 5169 |
| Q6P0N0_C890 | MIS18BP1 Mis18-binding protein 1 | 5170 |
| Q5SW96_C286 | LDLRAP1 Low density lipoprotein | 5171 |
| | receptor adapter protein 1 | |
| Q96FW1_C212 | OTUB1 Ubiquitin thioesterase OTUB1 | 5172 |
| Q7Z7H8_C180 | MRPL10 39S ribosomal protein L10, | 5173 |
| | mitochondrial | |
| P21266_C208 | GSTM3 Glutathione S-transferase Mu 3 | 5174 |
| Q8N5F7_C47 | NKAP NF-kappa-B-activating protein | 5175 |
| Q00534_C83 | CDK6 Cyclin-dependent kinase 6 | 5176 |
| Q00537_C233 | CDK17 Cyclin-dependent kinase 17 | 5177 |
| Q13838_C300 | DDX39B Spliceosome RNA helicase | 5178 |
| | DDX39B | |
| Q9HB09_C266 | BCL2L12 Bcl-2-like protein 12 | 5179 |
| P13693_C172 | TPT1 Translationally-controlled tumor | 5180 |
| | protein | |
| Q9H6Q4_C300 | NARFL Cytosolic Fe—S cluster | 5181 |
| | assembly factor NARFL | |
| P05388_C226 | RPLP0 60S acidic ribosomal protein P0 | 5182 |
| P16885_C937 | PLCG2 1-phosphatidylinositol 4,5- | 5183 |
| | bisphosphate phosphodiesterase | |
| P16885_C1082 | PLCG2 1-phosphatidylinositol 4,5- | 5184 |
| | bisphosphate phosphodiesterase | |
| Q13905_C474 | RAPGEF1 Rap guanine nucleotide | 5185 |
| | exchange factor 1 | |
| O75439_C485 | PMPCB Mitochondrial-processing | 5186 |
| | peptidase subunit beta | |
| P43034_C168 | PAFAH1B1 Platelet-activating factor | 5187 |
| | acetylhydrolase IB subunit | |
| A6NDG6_C35 | PGP Phosphoglycolate phosphatase | 5188 |
| A6NDG6_C217 | PGP Phosphoglycolate phosphatase | 5189 |
| O60610_C314 | DIAPH1 Protein diaphanous homolog 1 | 5190 |
| Q92598_C167 | HSPH1 Heat shock protein 105 kDa | 5191 |
| Q13011_C92 | ECH1 Delta(3,5)-Delta(2,4)-dienoyl- | 5192 |
| | CoA isomerase, mitochondrial | |
| Q8NEM2_C591 | SHCBP1 SHC SH2 domain-binding | 5193 |
| | protein 1 | |
| Q9NRH3_C13 | TUBG2 Tubulin gamma-2 chain | 5194 |
| Q8NFC6_C1849 | BOD1L1 Biorientation of chromosomes | 5195 |
| | in cell division protein 1-like 1 | |
| Q7L8L6_C670 | FASTKD5 FAST kinase domain- | 5196 |
| | containing protein 5 | |
| Q13123_C263 | IK Protein Red | 5197 |
| Q9UPN3_C666 | MACF1 Microtubule-actin cross- | 5198 |
| | linking factor 1, isoforms | |
| Q9UPN3_C7245 | MACF1 Microtubule-actin cross- | 5199 |
| | linking factor 1, isoforms | |
| Q9UPN7_C689 | PPP6R1 Serine/threonine-protein | 5200 |
| | phosphatase 6 regulatory | |
| Q9UGP4_C374 | LIMD1 LIM domain-containing protein | 5201 |
| | 1 | |
| Q9UGP4_C401 | LIMD1 LIM domain-containing protein | 5202 |
| | 1 | |
| O75369_C183 | FLNB Filamin-B | 5203 |
| O75369_C604 | FLNB Filamin-B | 5204 |
| P40763_C687 | STAT3 Signal transducer and activator | 5205 |
| | of transcription 3 | |
| Q13347_C160 | EIF3I Eukaryotic translation initiation | 5206 |
| | factor 3 subunit | |
| Q96KG9_C210 | SCYL1 N-terminal kinase-like protein | 5207 |
| Q96KG9_C241 | SCYL1 N-terminal kinase-like protein | 5208 |
| P21980_C27 | TGM2 Protein-glutamine gamma- | 5209 |
| | glutamyltransferase 2 | |
| P21980_C524 | TGM2 Protein-glutamine gamma- | 5210 |
| | glutamyltransferase 2 | |
| Q9ULJ6_C81 | ZMIZ1 Zinc finger MIZ domain- | 5211 |
| | containing protein 1 | |
| P30519_C265 | HMOX2 Heme oxygenase 2 | 5212 |
| Q96RE7_C178 | NACC1 Nucleus accumbens-associated | 5213 |
| | protein 1 | |
| Q96RE7_C301 | NACC1 Nucleus accumbens-associated | 5214 |
| | protein 1 | |
| Q96GX9_C32 | APIP Probable methylthioribulose-1- | 5215 |
| | phosphate dehydratase | |
| P49792_C1335 | RANBP2 E3 SUMO-protein ligase | 5216 |
| | RanBP2 | |
| P49792_C2696 | RANBP2 E3 SUMO-protein ligase | 5217 |
| | RanBP2 | |
| P56545_C60 | CTBP2 C-terminal-binding protein 2 | 5218 |
| Q5SRE5_C1433 | NUP188 Nucleoporin NUP188 | 5219 |
| | homolog | |
| Q9P0L0_C128 | VAPA Vesicle-associated membrane | 5220 |
| | protein-associated protein A, 33 kDa | |
| Q9Y618_C2447 | NCOR2 Nuclear receptor corepressor 2 | 5221 |
| Q9Y617_C175 | PSAT1 Phosphoserine aminotransferase | 5222 |
| Q9Y617_C245 | PSAT1 Phosphoserine aminotransferase | 5223 |
| Q9UIA9_C244 | XPO7 Exportin-7 | 5224 |
| Q9H8U3_C118 | ZFAND3 AN1-type zinc finger protein | 5225 |
| | 3 | |
| O00506_C73 | STK25 Serine/threonine-protein kinase | 5226 |
| | 25 | |
| Q4G0N4_C311 | NADKD1 NAD kinase domain- | 5227 |
| | containing protein 1 | |
| Q4V328_C744 | GRIPAP1 GRIP1-associated protein 1 | 5228 |
| Q9BXP5_C479 | SRRT Serrate RNA effector molecule | 5229 |
| | homolog | |
| Q01581_C268 | HMGCS1 Hydroxymethylglutaryl-CoA | 5230 |
| | synthase, cytoplasmic | |
| P46013_C643 | MKI67 Antigen KI-67 | 5231 |
| P17858_C351 | PFKL 6-phosphofructokinase, liver | 5232 |
| | type | |
| Q9H7Z7_C110 | PTGES2 Prostaglandin E synthase 2 | 5233 |
| O75083_C194 | WDR1 WD repeat-containing protein 1 | 5234 |
| O43252_C127 | PAPSS1 Bifunctional 3- | 5235 |
| | phosphoadenosine 5-phosphosulfate | |
| | synthase 1 | |
| Q09028_C278 | RBBP4 Histone-binding protein | 5236 |
| | RBBP4 | |
| Q8WXH0_C1235 | SYNE2 Nesprin-2 | 5237 |
| Q9UPM8_C1119 | AP4E1 AP-4 complex subunit epsilon-1 | 5238 |
| P23368_C428 | ME2 NAD-dependent malic enzyme, | 5239 |
| | mitochondrial | |
| O95402_C598 | MED26 Mediator of RNA polymerase | 5240 |
| | II transcription subunit | |
| Q8TD19_C808 | NEK9 Serine/threonine-protein kinase | 5241 |
| | Nek9 | |
| P11216_C109 | PYGB Glycogen phosphorylase, brain | 5242 |
| | form | |
| P40938_C11 | RFC3 Replication factor C subunit 3 | 5243 |
| P40939_C110 | HADHA Trifunctional enzyme subunit | 5244 |
| | alpha, mitochondrial | |
| P40939_C550 | HADHA Trifunctional enzyme subunit | 5245 |
| | alpha, mitochondrial | |
| Q8NCW5_C127 | APOA1BP NAD(P)H-hydrate | 5246 |
| | epimerase | |
| Q13356_C15 | PPIL2 Peptidyl-prolyl cis-trans | 5247 |
| | isomerase-like 2 | |
| Q5F1R6_C3 | DNAJC21 DnaJ homolog subfamily C | 5248 |
| | member 21 | |
| P40337_C77 | VHL Von Hippel-Lindau disease tumor | 5249 |
| | suppressor | |
| Q8N6R0_C226 | METTL13 Methyltransferase-like | 5250 |
| | protein 13 | |
| P39023_C336 | RPL3 60S ribosomal protein L3 | 5251 |
| P51610_C135 | HCFC1 Host cell factor 1 | 5252 |
| Q12800_C453 | TFCP2 Alpha-globin transcription | 5253 |
| | factor CP2 | |
| Q9Y281_C39 | CFL2 Cofilin-2 | 5254 |
| Q5T4S7_C1274 | UBR4 E3 ubiquitin-protein ligase | 5255 |
| | UBR4 | |
| Q5T4S7_C2222 | UBR4 E3 ubiquitin-protein ligase | 5256 |
| | UBR4 | |
| Q5T4S7_C4049 | UBR4 E3 ubiquitin-protein ligase | 5257 |
| | UBR4 | |
| O00154_C117 | ACOT7 Cytosolic acyl coenzyme A | 5258 |
| | thioester hydrolase | |
| Q53EZ4_C159 | CEP55 Centrosomal protein of 55 kDa | 5259 |
| Q5VV42_C138 | CDKAL1 Threonylcarbamoyladenosine | 5260 |
| | tRNA methylthiotransferase | |
| Q9P2A4_C145 | ABI3 ABI gene family member 3 | 5261 |
| Q9BRR8_C839 | GPATCH1 G patch domain-containing | 5262 |
| | protein 1 | |
| Q9Y3I0_C122 | C22orf28 tRNA-splicing ligase RtcB | 5263 |
| | homolog | |
| P28331_C92 | NDUFS1 NADH-ubiquinone | 5264 |
| | oxidoreductase 75 kDa subunit, | |
| | mitochondrial | |
| P62826_C122 | RAN GTP-binding nuclear protein Ran | 5265 |
| Q9BZE1_C153 | MRPL37 39S ribosomal protein L37, | 5266 |
| | mitochondrial | |
| P01033_C197 | TIMP1 Metalloproteinase inhibitor 1 | 5267 |
| Q99661_C610 | KIF2C Kinesin-like protein KIF2C | 5268 |
| Q99666_C1601 | RGPD6 RANBP2-like and GRIP | 5269 |
| | domain-containing protein 5/6 | |
| P24468_C343 | NR2F2 COUP transcription factor 2 | 5270 |
| O60701_C288 | UGDH UDP-glucose 6-dehydrogenase | 5271 |
| O43488_C214 | AKR7A2 Aflatoxin B1 aldehyde | 5272 |
| | reductase member 2 | |
| P53618_C102 | COPB1 Coatomer subunit beta | 5273 |
| Q8WV24_C148 | PHLDA1 Pleckstrin homology-like | 5274 |
| | domain family A member 1 | |
| Q8WV24_C266 | PHLDA1 Pleckstrin homology-like | 5275 |
| | domain family A member 1 | |
| Q8TEX9_C483 | IPO4 Importin-4 | 5276 |
| O95433_C301 | AHSA1 Activator of 90 kDa heat shock | 5277 |
| | protein ATPase homolog | |
| P49327_C1992 | FASN Fatty acid synthase | 5278 |
| Q99447_C30 | PCYT2 Ethanolamine-phosphate | 5279 |
| | cytidylyltransferase | |
| P49321_C254 | NASP Nuclear autoantigenic sperm | 5280 |
| | protein | |
| P14618_C165 | PKM Pyruvate kinase isozymes M1/M2 | 5281 |
| Q05655_C393 | PRKCD Protein kinase C delta type | 5282 |
| Q14012_C179 | CAMK1 Calcium/calmodulin- | 5283 |
| | dependent protein kinase type 1 | |
| Q15746_C1077 | MYLK Myosin light chain kinase, | 5284 |
| | smooth muscle | |
| O43353_C414 | RIPK2 Receptor-interacting | 5285 |
| | serine/threonine-protein kinase 2 | |
| P41091_C434 | EIF2S3 Eukaryotic translation initiation | 5286 |
| | factor 2 subunit | |
| Q9BQ39_C603 | DDX50 ATP-dependent RNA helicase | 5287 |
| | DDX50 | |
| P13489_C38 | RNH1 Ribonuclease inhibitor | 5288 |
| Q8TBC4_C70 | UBA3 NEDD8-activating enzyme E1 | 5289 |
| | catalytic subunit | |
| P25325_C65 | MPST 3-mercaptopyruvate | 5290 |
| | sulfurtransferase | |
| O15287_C392 | FANCG Fanconi anemia group G | 5291 |
| | protein | |
| P13804_C109 | ETFA Electron transfer flavoprotein | 5292 |
| | subunit alpha, mitochondrial | |
| P31153_C149 | MAT2A S-adenosylmethionine | 5293 |
| | synthase isoform type-2 | |
| Q86V48_C594 | LUZP1 Leucine zipper protein 1 | 5294 |
| Q9UKU7_C351 | ACAD8 Isobutyryl-CoA | 5295 |
| | dehydrogenase, mitochondrial | |
| Q12888_C772 | TP53BP1 Tumor suppressor p53- | 5296 |
| | binding protein 1 | |
| Q12888_C1796 | TP53BP1 Tumor suppressor p53- | 5297 |
| | binding protein 1 | |
| P14923_C511 | JUP Junction plakoglobin | 5298 |
| Q8NBJ5_C203 | GLT25D1 Procollagen | 5299 |
| | galactosyltransferase 1 | |
| Q7LDG7_C398 | RASGRP2 RAS guanyl-releasing | 5300 |
| | protein 2 | |
| Q56VL3_C27 | OCIAD2 OCIA domain-containing | 5301 |
| | protein 2 | |
| P01591_C123 | IGJ Immunoglobulin J chain | 5302 |
| Q9H1B7_C298 | IRF2BPL Interferon regulatory factor | 5303 |
| | 2-binding protein-lik | |
| O00522_C134 | KRIT1 Krev interaction trapped protein | 5304 |
| | 1 | |
| Q9BZZ5_C234 | API5 Apoptosis inhibitor 5 | 5305 |
| Q9H8G2_C226 | CAAP1 Caspase activity and apoptosis | 5306 |
| | inhibitor 1 | |
| O60711_C379 | LPXN Leupaxin | 5307 |
| Q6KC79_C279 | NIPBL Nipped-B-like protein | 5308 |
| Q92974_C335 | ARHGEF2 Rho guanine nucleotide | 5309 |
| | exchange factor 2 | |
| P43007_C109 | SLC1A4 Neutral amino acid transporter | 5310 |
| | A | |
| Q92973_C164 | TNPO1 Transportin-1 | 5311 |
| Q92973_C862 | TNPO1 Transportin-1 | 5312 |
| P54578_C359 | USP14 Ubiquitin carboxyl-terminal | 5313 |
| | hydrolase 14 | |
| Q92576_C561 | PHF3 PHD finger protein 3 | 5314 |
| Q13200_C285 | PSMD2 26S proteasome non-ATPase | 5315 |
| | regulatory subunit 2 | |
| P49336_C349 | CDK8 Cyclin-dependent kinase 8 | 5316 |
| Q8IVH4_C184 | MMAA Methylmalonic aciduria type A | 5317 |
| | protein, mitochondrial | |
| Q9GZR7_C832 | DDX24 ATP-dependent RNA helicase | 5318 |
| | DDX24 | |
| Q15149_C888 | PLEC Plectin | 5319 |
| Q15149_C1405 | PLEC Plectin | 5320 |
| Q15149_C3493 | PLEC Plectin | 5321 |
| Q15149_C3667 | PLEC Plectin | 5322 |
| P25787_C213 | PSMA2 Proteasome subunit alpha type- | 5323 |
| | 2 | |
| Q9ULW0_C198 | TPX2 Targeting protein for Xklp2 | 5324 |
| Q9UN19_C54 | DAPP1 Dual adapter for | 5325 |
| | phosphotyrosine and 3-phosphotyrosine | |
| | and 3-phosphoinositide | |
| P40261_C115 | NNMT Nicotinamide N- | 5326 |
| | methyltransferase | |
| Q9BWJ5_C41 | SF3B5 Splicing factor 3B subunit 5 | 5327 |
| Q9BR76_C345 | CORO1B Coronin-1B | 5328 |
| Q9HB40_C191 | SCPEP1 Retinoid-inducible serine | 5329 |
| | carboxypeptidase | |
| Q9BPZ3_C60 | PAIP2 Polyadenylate-binding protein- | 5330 |
| | interacting protein | |
| Q96F07_C1265 | CYFIP2 Cytoplasmic FMR1-interacting | 5331 |
| | protein 2 | |
| Q8TCG1_C583 | KIAA1524 Protein CIP2A | 5332 |
| O00442_C100 | RTCA RNA 3-terminal phosphate | 5333 |
| | cyclase | |
| P06132_C308 | UROD Uroporphyrinogen | 5334 |
| | decarboxylase | |
| Q9NTJ3_C260 | SMC4 Structural maintenance of | 5335 |
| | chromosomes protein 4 | |
| Q86UU0_C68 | BCL9L B-cell CLL/lymphoma 9-like | 5336 |
| | protein | |
| Q15031_C167 | LARS2 Probable leucine--tRNA ligase, | 5337 |
| | mitochondrial | |
| O15164_C394 | TRIM24 Transcription intermediary | 5338 |
| | factor 1-alpha | |
| Q5VT52_C222 | RPRD2 Regulation of nuclear pre- | 5339 |
| | mRNA domain-containing protein 2 | |
| P22314_C179 | UBA1 Ubiquitin-like modifier- | 5340 |
| | activating enzyme 1 | |
| Q53GQ0_C215 | HSD17B12 Estradiol 17-beta- | 5341 |
| | dehydrogenase 12 | |
| P78527_C223 | PRKDC DNA-dependent protein kinase | 5342 |
| | catalytic subunit | |
| P78527_C1629 | PRKDC DNA-dependent protein kinase | 5343 |
| | catalytic subunit | |
| P42566_C824 | EPS15 Epidermal growth factor | 5344 |
| | receptor substrate 15 | |
| Q5VUJ6_C256 | LRCH2 Leucine-rich repeat and | 5345 |
| | calponin homology domain-containing | |
| | 2 | |
| Q9H4I2_C11 | ZHX3 Zinc fingers and homeoboxes | 5346 |
| | protein 3 | |
| Q9H4I2_C335 | ZHX3 Zinc fingers and homeoboxes | 5347 |
| | protein 3 | |
| P42695_C631 | NCAPD3 Condensin-2 complex subunit | 5348 |
| | D3 | |
| Q96HE7_C104 | ERO1L ERO1-like protein alpha | 5349 |
| Q53HL2_C273 | CDCA8 Borealin | 5350 |
| Q9Y2L5_C265 | TRAPPC8 Trafficking protein particle | 5351 |
| | complex subunit 8 | |
| Q04721_C2085 | NOTCH2 Neurogenic locus notch | 5352 |
| | homolog protein 2 | |
| Q04726_C26 | TLE3 Transducin-like enhancer protein | 5353 |
| | 3 | |
| Q6P2E9_C838 | EDC4 Enhancer of mRNA-decapping | 5354 |
| | protein 4 | |
| Q99567_C454 | NUP88 Nuclear pore complex protein | 5355 |
| | Nup88 | |
| P49189_C45 | ALDH9A1 4- | 5356 |
| | trimethylaminobutyraldehyde | |
| | dehydrogenase | |
| Q14789_C3070 | GOLGB1 Golgin subfamily B member | 5357 |
| | 1 | |
| Q14789_C3144 | GOLGB1 Golgin subfamily B member | 5358 |
| | 1 | |
| O43592_C38 | XPOT Exportin-T | 5359 |
| O43592_C938 | XPOT Exportin-T | 5360 |
| Q9NUQ8_C102 | ABCF3 ATP-binding cassette sub- | 5361 |
| | family F member 3 | |
| P20810_C328 | CAST Calpastatin | 5362 |
| P33176_C174 | KIF5B Kinesin-1 heavy chain | 5363 |
| Q9GZQ3_C117 | COMMD5 COMM domain-containing | 5364 |
| | protein 5 | |
| P49454_C2415 | CENPF Centromere protein F | 5365 |
| Q76N89_C1574 | HECW1 E3 ubiquitin-protein ligase | 5366 |
| | HECW1 | |
| Q9NQZ5_C362 | STARD7 StAR-related lipid transfer | 5367 |
| | protein 7, mitochondrial | |
| Q9BQ15_C99 | NABP2 SOSS complex subunit B1 | 5368 |
| Q12905_C37 | ILF2 Interleukin enhancer-binding | 5369 |
| | factor 2 | |
| P04406_C158 | GAPDH Glyceraldehyde-3-phosphate | 5370 |
| | dehydrogenase | |
| P11177_C306 | PDHB Pyruvate dehydrogenase E1 | 5371 |
| | component subunit beta, | |
| E7EQZ4_C60 | SMN1 Survival motor neuron protein | 5372 |
| Q6UN15_C216 | FIP1L1 Pre-mRNA 3-end-processing | 5373 |
| | factor FIP1 | |
| Q96GJ1_C289 | TRMT2B tRNA (uracil(54)-C(5))- | 5374 |
| | methyltransferase homolog | |
| Q9UEW8_C450 | STK39 STE20/SPS1-related proline- | 5375 |
| | alanine-rich protein kinase | |
| Q15785_C236 | TOMM34 Mitochondrial import | 5376 |
| | receptor subunit TOM34 | |
| Q5T160_C11 | RARS2 Probable arginine--tRNA | 5377 |
| | ligase, mitochondrial | |
| P40692_C142 | MLH1 DNA mismatch repair protein | 5378 |
| | Mlh1 | |
| Q15283_C314 | RASA2 Ras GTPase-activating protein | 5379 |
| | 2 | |
| Q96I25_C302 | RBM17 Splicing factor 45 | 5380 |
| Q96I25_C385 | RBM17 Splicing factor 45 | 5381 |
| Q15334_C505 | LLGL1 Lethal(2) giant larvae protein | 5382 |
| | homolog 1 | |
| Q712K3_C191 | UBE2R2 Ubiquitin-conjugating | 5383 |
| | enzyme E2 R2 | |
| O95373_C90 | IPO7 Importin-7 | 5384 |
| Q9BV44_C426 | THUMPD3 THUMP domain- | 5385 |
| | containing protein 3 | |
| O14578_C105 | CIT Citron Rho-interacting kinase | 5386 |
| O14579_C34 | COPE Coatomer subunit epsilon | 5387 |
| Q9NXW9_C204 | ALKBH4 Probable alpha-ketoglutarate- | 5388 |
| | dependent dioxygenase | |
| Q9Y2H0_C736 | DLGAP4 Disks large-associated protein | 5389 |
| | 4 | |
| Q96CV9_C239 | OPTN Optineurin | 5390 |
| Q9Y3L3_C102 | SH3BP1 SH3 domain-binding protein 1 | 5391 |
| Q96EV2_C1093 | RBM33 RNA-binding protein 33 | 5392 |
| Q9UM54_C1102 | MYO6 Unconventional myosin-VI | 5393 |
| Q15027_C64 | ACAP1 Arf-GAP with coiled-coil, | 5394 |
| | ANK repeat and PH domain | |
| O00541_C391 | PES1 Pescadillo homolog | 5395 |
| Q9Y277_C36 | VDAC3 Voltage-dependent anion- | 5396 |
| | selective channel protein | |
| Q9Y277_C229 | VDAC3 Voltage-dependent anion- | 5397 |
| | selective channel protein | |
| Q8NI27_C743 | THOC2 THO complex subunit 2 | 5398 |
| O76094_C349 | SRP72 Signal recognition particle 72 | 5399 |
| | kDa protein | |
| Q14315_C1348 | FLNC Filamin-C | 5400 |
| Q14315_C1735 | FLNC Filamin-C | 5401 |
| Q09666_C1967 | AHNAK Neuroblast differentiation- | 5402 |
| | associated protein AHNA | |
| P14621_C22 | ACYP2 Acylphosphatase-2 | 5403 |
| Q12974_C101 | PTP4A2 Protein tyrosine phosphatase | 5404 |
| | type IVA 2 | |
| O75334_C143 | PPFIA2 Liprin-alpha-2 | 5405 |
| O14979_C277 | HNRPDL Heterogeneous nuclear | 5406 |
| | ribonucleoprotein D-like | |
| Q8WUM0_C1037 | NUP133 Nuclear pore complex protein | 5407 |
| | Nup133 | |
| Q96PN7_C876 | TRERF1 Transcriptional-regulating | 5408 |
| | factor 1 | |
| Q9H9A6_C54 | LRRC40 Leucine-rich repeat- | 5409 |
| | containing protein 40 | |
| Q01167_C357 | FOXK2 Forkhead box protein K2 | 5410 |
| Q13310_C128 | PABPC4 Polyadenylate-binding protein | 5411 |
| | 4 | |
| P06493_C119 | CDK1 Cyclin-dependent kinase 1 | 5412 |
| P62937_C52 | PPIA Peptidyl-prolyl cis-trans | 5413 |
| | isomerase A | |
| Q9UBQ5_C190 | EIF3K Eukaryotic translation initiation | 5414 |
| | factor 3 subunit | |
| Q9NY93_C298 | DDX56 Probable ATP-dependent RNA | 5415 |
| | helicase DDX56 | |
| P50453_C370 | SERPINB9 Serpin B9 | 5416 |
| P12236_C160 | SLC25A6 ADP/ATP translocase 3 | 5417 |
| Q6ZRQ5_C1068 | MMS22L Protein MMS22-like | 5418 |
| Q9BYI3_C371 | FAM126A Hyccin | 5419 |
| P51148_C64 | RAB5C Ras-related protein Rab-5C | 5420 |
| P09110_C87 | ACAA1 3-ketoacyl-CoA thiolase, | 5421 |
| | peroxisomal | |
| Q9UL15_C118 | BAG5 BAG family molecular | 5422 |
| | chaperone regulator 5 | |
| Q9UL12_C497 | SARDH Sarcosine dehydrogenase, | 5423 |
| | mitochondrial | |
| Q96CG3_C169 | TIFA TRAF-interacting protein with | 5424 |
| | FHA domain-containing | |
| O60841_C853 | EIF5B Eukaryotic translation initiation | 5425 |
| | factor 5B | |
| Q15306_C19 | IRF4 Interferon regulatory factor 4 | 5426 |
| Q9Y265_C94 | RUVBL1 RuvB-like 1 | 5427 |
| Q9Y263_C193 | PLAA Phospholipase A-2-activating | 5428 |
| | protein | |
| Q9UBZ4_C27 | APEX2 DNA-(apurinic or apyrimidinic | 5429 |
| | site) lyase 2 | |
| O75528_C255 | TADA3 Transcriptional adapter 3 | 5430 |
| Q14997_C710 | PSME4 Proteasome activator complex | 5431 |
| | subunit 4 | |
| Q9P107_C895 | GMIP GEM-interacting protein | 5432 |
| P35579_C740 | MYH9 Myosin-9 | 5433 |
| Q8TAQ2_C91 | SMARCC2 SWI/SNF complex subunit | 5434 |
| | SMARCC2 | |
| O14966_C120 | RAB7L1 Ras-related protein Rab-7L1 | 5435 |
| Q13325_C137 | IFIT5 Interferon-induced protein with | 5436 |
| | tetratricopeptide | |
| Q7Z7A3_C290 | CTU1 Cytoplasmic tRNA 2-thiolation | 5437 |
| | protein 1 | |
| Q9ULT8_C2579 | HECTD1 E3 ubiquitin-protein ligase | 5438 |
| | HECTD1 | |
| Q0VG06_C15 | FAAP100 Fanconi anemia-associated | 5439 |
| | protein of 100 kDa | |
| Q13439_C1253 | GOLGA4 Golgin subfamily A member | 5440 |
| | 4 | |
| Q9UPT9_C494 | USP22 Ubiquitin carboxyl-terminal | 5441 |
| | hydrolase 22 | |
| Q9HCJ3_C362 | RAVER2 Ribonucleoprotein PTB- | 5442 |
| | binding 2 | |
| O60306_C28 | AQR Intron-binding protein aquarius | 5443 |
| Q92879_C61 | CELF1 CUGBP Elav-like family | 5444 |
| | member 1 | |
| P10620_C50 | MGST1 Microsomal glutathione S- | 5445 |
| | transferase 1 | |
| P01892_C125 | HLA-A HLA class I histocompatibility | 5446 |
| | antigen, A-2 alpha | |
| Q9Y312_C370 | AAR2 Protein AAR2 homolog | 5447 |
| P55265_C392 | ADAR Double-stranded RNA-specific | 5448 |
| | adenosine deaminase | |
| P60900_C78 | PSMA6 Proteasome subunit alpha type- | 5449 |
| | 6 | |
| Q63ZY3_C249 | KANK2 KN motif and ankyrin repeat | 5450 |
| | domain-containing protein 2 | |
| Q63ZY3_C412 | KANK2 KN motif and ankyrin repeat | 5451 |
| | domain-containing protein | |
| O95159_C230 | ZFPL1 Zinc finger protein-like 1 | 5452 |
| P62873_C294 | GNB1 Guanine nucleotide-binding | 5453 |
| | protein G(I)/G(S)/G(T) | |
| O00567_C211 | NOP56 Nucleolar protein 56 | 5454 |
| Q15003_C397 | NCAPH Condensin complex subunit 2 | 5455 |
| Q9C0C2_C136 | TNKS1BP1 182 kDa tankyrase-1- | 5456 |
| | binding protein | |
| Q9C0C9_C1099 | UBE2O Ubiquitin-conjugating enzyme | 5457 |
| | E2 O | |
| Q12841_C233 | FSTL1 Follistatin-related protein 1 | 5458 |
| O75533_C1244 | SF3B1 Splicing factor 3B subunit 1 | 5459 |
| O75534_C106 | CSDE1 Cold shock domain-containing | 5460 |
| | protein E1 | |
| E7EVK2_C211 | MTG1 Mitochondrial GTPase 1 | 5461 |
| Q13158_C168 | FADD Protein FADD | 5462 |
| Q9NRA8_C125 | EIF4ENIF1 Eukaryotic translation | 5463 |
| | initiation factor 4E transporter | |
| Q13155_C205 | AIMP2 Aminoacyl tRNA synthase | 5464 |
| | complex-interacting multifunctional | |
| | protein 2 | |
| Q14192_C10 | FHL2 Four and a half LIM domains | 5465 |
| | protein 2 | |
| Q8TEU7_C93 | RAPGEF6 Rap guanine nucleotide | 5466 |
| | exchange factor 6 | |
| Q14204_C978 | DYNC1H1 Cytoplasmic dynein 1 | 5467 |
| | heavy chain 1 | |
| Q14204_C4644 | DYNC1H1 Cytoplasmic dynein 1 | 5468 |
| | heavy chain 1 | |
| O60684_C296 | KPNA6 Importin subunit alpha-7 | 5469 |
| Q92844_C318 | TANK TRAF family member- | 5470 |
| | associated NF-kappa-B activator | |
| Q9Y4H4_C160 | GPSM3 G-protein-signaling modulator | 5471 |
| | 3 | |
| Q8WXI9_C423 | GATAD2B Transcriptional repressor | 5472 |
| | p66-beta | |
| P22102_C93 | GART Trifunctional purine | 5473 |
| | biosynthetic protein adenosine | |
| P32320_C102 | CDA Cytidine deaminase | 5474 |
| Q13085_C1116 | ACACA Acetyl-CoA carboxylase 1 | 5475 |
| Q14511_C18 | NEDD9 Enhancer of filamentation 1 | 5476 |
| O95551_C273 | TDP2 Tyrosyl-DNA phosphodiesterase | 5477 |
| | 2 | |
| Q13330_C68 | MTA1 Metastasis-associated protein | 5478 |
| | MTA1 | |
| A6NJ78_C172 | METTL15 Probable methyltransferase- | 5479 |
| | like protein 15 | |
| O43491_C424 | EPB41L2 Band 4.1-like protein 2 | 5480 |
| P82663_C141 | MRPS25 28S ribosomal protein S25, | 5481 |
| | mitochondrial | |
| Q13428_C1012 | TCOF1 Treacle protein | 5482 |
| Q5JSP0_C43 | FGD3 FYVE, RhoGEF and PH | 5483 |
| | domain-containing protein 3 | |
| Q14847_C32 | LASP1 LIM and SH3 domain protein 1 | 5484 |
| Q9UHB7_C315 | AFF4 AF4/FMR2 family member 4 | 5485 |
| P48735_C402 | IDH2 Isocitrate dehydrogenase | 5486 |
| Q2TAL8_C529 | QRICH1 Glutamine-rich protein 1 | 5487 |
| Q9UJM3_C124 | ERRFI1 ERBB receptor feedback | 5488 |
| | inhibitor 1 | |
| Q7L1V2_C87 | MON1B Vacuolar fusion protein | 5489 |
| | MON1 homolog B | |
| Q96KP4_C205 | CNDP2 Cytosolic non-specific | 5490 |
| | dipeptidase | |
| P62136_C62 | PPP1CA Serine/threonine-protein | 5491 |
| | phosphatase PP1-alpha catalytic subunit | |
| | alpha | |
| P13984_C116 | GTF2F2 General transcription factor | 5492 |
| | IIF subunit 2 | |
| Q7L775_C337 | EPM2AIP1 EPM2A-interacting protein | 5493 |
| | 1 | |
| P55209_C258 | NAP1L1 Nucleosome assembly protein | 5494 |
| | 1-like 1 | |
| Q9NQR4_C146 | NIT2 Omega-amidase NIT2 | 5495 |
| Q13459_C1279 | MYO9B Unconventional myosin-IXb | 5496 |
| Q9NVX0_C118 | HAUS2 HAUS augmin-like complex | 5497 |
| | subunit 2 | |
| Q969S9_C248 | GFM2 Ribosome-releasing factor 2, | 5498 |
| | mitochondrial | |
| P14866_C260 | HNRNPL Heterogeneous nuclear | 5499 |
| | ribonucleoprotein L | |
| Q13569_C233 | TDG G/T mismatch-specific thymine | 5500 |
| | DNA glycosylase | |
| O95239_C28 | KIF4A Chromosome-associated kinesin | 5501 |
| | KIF4A | |
| Q71RC2_C599 | LARP4 La-related protein 4 | 5502 |
| Q71RC2_C623 | LARP4 La-related protein 4 | 5503 |
| P29590_C80 | PML Protein PML | 5504 |
| P29590_C151 | PML Protein PML | 5505 |
| P29590_C189 | PML Protein PML | 5506 |
| P29590_C227 | PML Protein PML | 5507 |
| P29590_C479 | PML Protein PML | 5508 |
| Q09472_C1621 | EP300 Histone acetyltransferase p300 | 5509 |
| O15235_C93 | MRPS12 28S ribosomal protein S12, | 5510 |
| | mitochondrial | |
| P24534_C161 | EEF1B2 Elongation factor 1-beta | 5511 |
| P54819_C92 | AK2 Adenylate kinase 2, mitochondrial | 5512 |
| Q9Y333_C26 | LSM2 U6 snRNA-associated Sm-like | 5513 |
| | protein LSm2 | |
| Q9UER7_C427 | DAXX Death domain-associated | 5514 |
| | protein 6 | |
| Q3KQU3_C373 | MAP7D1 MAP7 domain-containing | 5515 |
| | protein 1 | |
| Q8N0W3_C129 | FUK L-fucose kinase | 5516 |
| Q16401_C361 | PSMD5 26S proteasome non-ATPase | 5517 |
| | regulatory subunit 5 | |
| Q99590_C478 | SCAF11 Protein SCAF11 | 5518 |
| Q9BYX2_C686 | TBC1D2 TBC1 domain family member | 5519 |
| | 2A | |
| A2A288_C64 | ZC3H12D Probable ribonuclease | 5520 |
| | ZC3H12D | |
| Q9UBR2_C214 | CTSZ Cathepsin Z | 5521 |
| Q5U5Q3_C319 | MEX3C RNA-binding E3 ubiquitin- | 5522 |
| | protein ligase MEX3C | |
| O95059_C31 | RPP14 Ribonuclease P protein subunit | 5523 |
| | p14 | |
| Q9NV56_C170 | MRGBP MRG-binding protein | 5524 |
| O14617_C574 | AP3D1 AP-3 complex subunit delta-1 | 5525 |
| Q14980_C1937 | NUMA1 Nuclear mitotic apparatus | 5526 |
| | protein 1 | |
| Q9BYV9_C698 | BACH2 Transcription regulator protein | 5527 |
| | BACH2 | |
| P55072_C184 | VCP Transitional endoplasmic | 5528 |
| | reticulum ATPase | |
| P55072_C209 | VCP Transitional endoplasmic | 5529 |
| | reticulum ATPase | |
| Q01433_C848 | AMPD2 AMP deaminase 2 | 5530 |
| P55211_C172 | CASP9 Caspase-9 | 5531 |
| Q08378_C455 | GOLGA3 Golgin subfamily A member | 5532 |
| | 3 | |
| Q12912_C264 | LRMP Lymphoid-restricted membrane | 5533 |
| | protein | |
| Q07021_C186 | C1QBP Complement component 1 Q | 5534 |
| | subcomponent-binding protein | |
| Q9NSE4_C414 | IARS2 Isoleucine--tRNA ligase, | 5535 |
| | mitochondrial | |
| Q9NSE4_C883 | IARS2 Isoleucine--tRNA ligase, | 5536 |
| | mitochondrial | |
| P14854_C30 | COX6B1 Cytochrome c oxidase | 5537 |
| | subunit 6B1 | |
| Q96ER3_C430 | SAAL1 Protein SAAL1 | 5538 |
| P08238_C592 | HSP90AB1 Heat shock protein HSP | 5539 |
| | 90-beta | |
| Q8IZ69_C551 | TRMT2A tRNA (uracil-5-)- | 5540 |
| | methyltransferase homolog A | |
| Q8NFX7_C120 | STXBP6 Syntaxin-binding protein 6 | 5541 |
| Q15118_C223 | PDK1 | 5542 |
| Q9P2J5_C1052 | LARS Leucine--tRNA ligase, | 5543 |
| | cytoplasmic | |
| Q9P2J5_C1053 | LARS Leucine--tRNA ligase, | 5544 |
| | cytoplasmic | |
| Q8TF72_C1331 | SHROOM3 Protein Shroom3 | 5545 |
| Q01415_C407 | GALK2 N-acetylgalactosamine kinase | 5546 |
| Q8IYL3_C124 | C1orf174 UPF0688 protein C1orf174 | 5547 |
| O43143_C644 | DHX15 Putative pre-mRNA-splicing | 5548 |
| | factor ATP-dependent RN | |
| Q99615_C247 | DNAJC7 DnaJ homolog subfamily C | 5549 |
| | member 7 | |
| Q05086_C843 | UBE3A Ubiquitin-protein ligase E3A | 5550 |
| P53667_C349 | LIMK1 LIM domain kinase 1 | 5551 |
| Q8IWV8_C1619 | UBR2 E3 ubiquitin-protein ligase | 5552 |
| | UBR2 | |
| Q96T51_C81 | RUFY1 RUN and FYVE domain- | 5553 |
| | containing protein 1 | |
| P13797_C209 | PLS3 Plastin-3 | 5554 |
| P39880_C802 | CUX1 Homeobox protein cut-like 1 | 5555 |
| Q53EL6_C288 | PDCD4 Programmed cell death protein | 5556 |
| | 4 | |
| P12277_C283 | CKB Creatine kinase B-type | 5557 |
| Q13617_C385 | CUL2 Cullin-2 | 5558 |
| Q8WXE1_C238 | ATRIP ATR-interacting protein | 5559 |
| Q8WXE1_C263 | ATRIP ATR-interacting protein | 5560 |
| Q7Z4Q2_C20 | HEATR3 HEAT repeat-containing | 5561 |
| | protein 3 | |
| Q15052_C564 | ARHGEF6 Rho guanine nucleotide | 5562 |
| | exchange factor 6 | |
| P49902_C181 | NT5C2 Cytosolic purine 5-nucleotidase | 5563 |
| P49903_C119 | SEPHS1 Selenide, water dikinase 1 | 5564 |
| Q53EP0_C442 | FNDC3B Fibronectin type III domain- | 5565 |
| | containing protein 3B | |
| Q92905_C218 | COPS5 COP9 signalosome complex | 5566 |
| | subunit 5 | |
| Q15121_C27 | PEA15 Astrocytic phosphoprotein | 5567 |
| | PEA-15 | |
| Q9Y315_C118 | DERA Putative deoxyribose-phosphate | 5568 |
| | aldolase | |
| O75909_C23 | CCNK Cyclin-K | 5569 |
| Q14232_C218 | EIF2B1 Translation initiation factor | 5570 |
| | eIF-2B subunit alpha | |
| P53582_C22 | METAP1 Methionine aminopeptidase 1 | 5571 |
| Q8WZ42_C5248 | TTN Titin | 5572 |
| Q6UB35_C828 | MTHFD1L Monofunctional C1- | 5573 |
| | tetrahydrofolate synthase, | |
| | mitochondrial | |
| O43516_C446 | WIPF1 WAS/WASL-interacting | 5574 |
| | protein family member 1 | |
| Q9UHB4_C486 | NDOR1 NADPH-dependent diflavin | 5575 |
| | oxidoreductase 1 | |
| P41252_C400 | IARS Isoleucine-tRNA ligase, | 5576 |
| | cytoplasmic | |
| Q9UHB6_C164 | LIMA1 LIM domain and actin-binding | 5577 |
| | protein 1 | |
| Q14141_C14 | SEPT6 Septin-6 | 5578 |
| Q14149_C307 | MORC3 MORC family CW-type zinc | 5579 |
| | finger protein 3 | |
| O75122_C528 | CLASP2 CLIP-associating protein 2 | 5580 |
| P46821_C905 | MAP1B Microtubule-associated protein | 5581 |
| | 1B | |
| P46821_C963 | MAP1B Microtubule-associated protein | 5582 |
| | 1B | |
| O60343_C295 | TBC1D4 TBC1 domain family member | 5583 |
| | 4 | |
| Q96C36_C232 | PYCR2 Pyrroline-5-carboxylate | 5584 |
| | reductase 2 | |
| P61011_C229 | SRP54 Signal recognition particle 54 | 5585 |
| | kDa protein | |
| Q9H223_C141 | EHD4 EH domain-containing protein 4 | 5586 |
| O14981_C1542 | BTAF1 TATA-binding protein- | 5587 |
| | associated factor 172 | |
| P13796_C283 | LCP1 Plastin-2 | 5588 |
| O95071_C691 | UBR5 E3 ubiquitin-protein ligase | 5589 |
| | UBR5 | |
| O95071_C2768 | UBR5 E3 ubiquitin-protein ligase | 5590 |
| | UBR5 | |
| Q9Y2L1_C476 | DIS3 Exosome complex exonuclease | 5591 |
| | RRP44 | |
| A0FGR8_C181 | ESYT2 Extended synaptotagmin-2 | 5592 |
| P09960_C275 | LTA4H Leukotriene A-4 hydrolase | 5593 |
| Q06330_C454 | RBPJ Recombining binding protein | 5594 |
| | suppressor of hairless | |
| O14519_C105 | CDK2AP1 Cyclin-dependent kinase 2- | 5595 |
| | associated protein 1 | |
| Q15545_C72 | TAF7 Transcription initiation factor | 5596 |
| | TFIID subunit 7 | |
| Q15545_C92 | TAF7 Transcription initiation factor | 5597 |
| | TFIID subunit 7 | |
| Q15543_C38 | TAF13 Transcription initiation factor | 5598 |
| | TFIID subunit 13 | |
| Q6P587_C22 | FAHD1 Acylpyruvase FAHD1, | 5599 |
| | mitochondrial | |
| P51649_C93 | ALDH5A1 Succinate-semialdehyde | 5600 |
| | dehydrogenase, mitochondrial | |
| Q15048_C15 | LRRC14 Leucine-rich repeat- | 5601 |
| | containing protein 14 | |
| Q15042_C117 | RAB3GAP1 Rab3 GTPase-activating | 5602 |
| | protein catalytic subunit | |
| Q15042_C873 | RAB3GAP1 Rab3 GTPase-activating | 5603 |
| | protein catalytic subunit | |
| Q6R327_C1631 | RICTOR Rapamycin-insensitive | 5604 |
| | companion of mTOR | |
| P49915_C530 | GMPS GMP synthase | 5605 |
| Q9Y2K7_C840 | KDM2A Lysine-specific demethylase | 5606 |
| | 2A | |
| P00973_C25 | OAS1 2-5-oligoadenylate synthase 1 | 5607 |
| Q01780_C651 | EXOSC10 Exosome component 10 | 5608 |
| P12268_C468 | IMPDH2 Inosine-5-monophosphate | 5609 |
| | dehydrogenase 2 | |
| P07437_C129 | TUBB Tubulin beta chain | 5610 |
| Q01658_C94 | DR1 Protein Dr1 | 5611 |
| Q9Y490_C29 | TLN1 Talin-1 | 5612 |
| Q9Y490_C1478 | TLN1 Talin-1 | 5613 |
| P43243_C821 | MATR3 Matrin-3 | 5614 |
| Q86W50_C253 | METTL16 Methyltransferase-like | 5615 |
| | protein 16 | |
| P49005_C447 | POLD2 DNA polymerase delta subunit | 5616 |
| | 2 | |
| Q9H3H3_C199 | C11orf68 UPF0696 protein C11orf68 | 5617 |
| P27708_C484 | CAD CAD protein | 5618 |
| Q709C8_C1481 | VPS13C Vacuolar protein sorting- | 5619 |
| | associated protein 13C | |
| P23497_C373 | SP100 Nuclear autoantigen Sp-100 | 5620 |
| Q9NXV2_C25 | KCTD5 BTB/POZ domain-containing | 5621 |
| | protein KCTD5 | |
| Q05209_C470 | PTPN12 Tyrosine-protein phosphatase | 5622 |
| | non-receptor type 12 | |
| Q9P258_C305 | RCC2 Protein RCC2 | 5623 |
| Q8N3R9_C490 | MPP5 MAGUK p55 subfamily member | 5624 |
| | 5 | |
| Q13464_C29 | ROCK1 Rho-associated protein kinase | 5625 |
| | 1 | |
| Q7L5Y9_C195 | MAEA Macrophage erythroblast | 5626 |
| | attacher | |
| O15446_C150 | CD3EAP DNA-directed RNA | 5627 |
| | polymerase I subunit RPA34 | |
| P54136_C36 | RARS Arginine--tRNA ligase, | 5628 |
| | cytoplasmic | |
| P54136_C638 | RARS Arginine--tRNA ligase, | 5629 |
| | cytoplasmic | |
| Q96F24_C155 | NRBF2 Nuclear receptor-binding factor | 5630 |
| | 2 | |
| Q9Y5L0_C527 | TNPO3 Transportin-3 | 5631 |
| Q86YV5_C444 | SGK223 Tyrosine-protein kinase | 5632 |
| | SgK223 | |
| O60502_C663 | MGEA5 Bifunctional protein NCOAT | 5633 |
| O60502_C896 | MGEA5 Bifunctional protein NCOAT | 5634 |
| Q13671_C223 | RIN1 Ras and Rab interactor 1 | 5635 |
| O95067_C131 | CCNB2 G2/mitotic-specific cyclin-B2 | 5636 |
| P38646_C608 | HSPA9 Stress-70 protein, | 5637 |
| | mitochondrial | |
| Q96MX6_C70 | WDR92 WD repeat-containing protein | 5638 |
| | 92 | |
| P49588_C901 | AARS Alanine--tRNA ligase, | 5639 |
| | cytoplasmic | |
| Q9HCC0_C391 | MCCC2 Methylcrotonoyl-CoA | 5640 |
| | carboxylase beta chain, mitochondrial | |
| P47755_C157 | CAPZA2 F-actin-capping protein | 5641 |
| | subunit alpha-2 | |
| Q8TDD1_C73 | DDX54 ATP-dependent RNA helicase | 5642 |
| | DDX54 | |
| P47897_C111 | QARS Glutamine--tRNA ligase | 5643 |
| P06730_C89 | EIF4E Eukaryotic translation initiation | 5644 |
| | factor 4E | |
| P06730_C136 | EIF4E Eukaryotic translation initiation | 5645 |
| | factor 4E | |
| Q7Z4W1_C150 | DCXR L-xylulose reductase | 5646 |
| Q96QB1_C532 | DLC1 Rho GTPase-activating protein 7 | 5647 |
| P30740_C344 | SERPINB1 Leukocyte elastase inhibitor | 5648 |
| O95758_C68 | PTBP3 Polypyrimidine tract-binding | 5649 |
| | protein 3 | |
| P00966_C132 | ASS1 Argininosuccinate synthase | 5650 |
| P62917_C115 | RPL8 60S ribosomal protein L8 | 5651 |
| Q00796_C106 | SORD Sorbitol dehydrogenase | 5652 |
| O75928_C346 | PIAS2 E3 SUMO-protein ligase PIAS2 | 5653 |
| Q8TF42_C367 | UBASH3B Ubiquitin-associated and | 5654 |
| | SH3 domain-containing protein | |
| Q9NVR7_C298 | TBCCD1 TBCC domain-containing | 5655 |
| | protein 1 | |
| Q9UK39_C302 | CCRN4L Nocturnin | 5656 |
| Q99627_C20 | COPS8 COP9 signalosome complex | 5657 |
| | subunit 8 | |
| Q9C0J8_C120 | WDR33 pre-mRNA 3 end processing | 5658 |
| | protein WDR33 | |
| Q6ZUJ8_C358 | PIK3AP1 Phosphoinositide 3-kinase | 5659 |
| | adapter protein 1 | |
| P23246_C431 | SFPQ Splicing factor, proline- and | 5660 |
| | glutamine-rich | |
| P48506_C501 | GCLC Glutamate--cysteine ligase | 5661 |
| | catalytic subunit | |
| Q04637_C821 | EIF4G1 Eukaryotic translation | 5662 |
| | initiation factor 4 gamma 1 | |
| P52701_C108 | MSH6 DNA mismatch repair protein | 5663 |
| | Msh6 | |
| P11310_C156 | ACADM Medium-chain specific acyl- | 5664 |
| | CoA dehydrogenase, mitochondrial | |
| Q96G03_C86 | PGM2 Phosphoglucomutase-2 | 5665 |
| Q9NZ32_C352 | ACTR10 Actin-related protein 10 | 5666 |
| P32119_C70 | PRDX2 Peroxiredoxin-2 | 5667 |
| Q5R3I4_C128 | TTC38 Tetratricopeptide repeat protein | 5668 |
| | 38 | |
| O43318_C513 | MAP3K7 Mitogen-activated protein | 5669 |
| | kinase kinase kinase 7 | |
| Q53H82_C21 | LACTB2 Beta-lactamase-like protein 2 | 5670 |
| P63165_C52 | SUMO1 Small ubiquitin-related | 5671 |
| | modifier 1 | |
| P33993_C566 | MCM7 DNA replication licensing | 5672 |
| | factor MCM7 | |
| P41567_C94 | EIF1 Eukaryotic translation initiation | 5673 |
| | factor 1 | |
| P22830_C395 | FECH Ferrochelatase, mitochondrial | 5674 |
| P36871_C160 | PGM1 Phosphoglucomutase-1 | 5675 |
| Q9H0D6_C29 | XRN2 5-3 exoribonuclease 2 | 5676 |
| P31751_C124 | AKT2 RAC-beta serine/threonine- | 5677 |
| | protein kinase | |
| P30622_C150 | CLIP1 CAP-Gly domain-containing | 5678 |
| | linker protein 1 | |
| Q06481_C133 | APLP2 Amyloid-like protein 2 | 5679 |
| Q9NR12_C311 | PDLIM7 PDZ and LIM domain protein | 5680 |
| | 7 | |
| A6NHR9_C505 | SMCHD1 Structural maintenance of | 5681 |
| | chromosomes flexible hinge domain | |
| | containing 1 | |
| A6NHR9_C1656 | SMCHD1 Structural maintenance of | 5682 |
| | chromosomes flexible hinge domain | |
| | containing 1 | |
| O75600_C134 | GCAT 2-amino-3-ketobutyrate | 5683 |
| | coenzyme A ligase, mitochondrial | |
| Q96IU2_C26 | ZBED3 Zinc finger BED domain- | 5684 |
| | containing protein 3 | |
| Q6PKG0_C1054 | LARP1 La-related protein 1 | 5685 |
| Q5VYS8_C343 | ZCCHC6 Terminal uridylyltransferase | 5686 |
| | 7 | |
| P35580_C382 | MYH10 Myosin-10 | 5687 |
| P49023_C546 | PXN Paxillin | 5688 |
| Q9NVS9_C72 | PNPO Pyridoxine-5-phosphate oxidase | 5689 |
| Q9NQG7_C547 | HPS4 Hermansky-Pudlak syndrome 4 | 5690 |
| | protein | |
| Q14289_C562 | PTK2B Protein-tyrosine kinase 2-beta | 5691 |
| Q96P47_C611 | AGAP3 Arf-GAP with GTPase, ANK | 5692 |
| | repeat and PH domain-containing | |
| | protein 3 | |
| Q92621_C921 | NUP205 Nuclear pore complex protein | 5693 |
| | Nup205 | |
| Q9BTA9_C615 | WAC WW domain-containing adapter | 5694 |
| | protein with coiled-coil | |
| Q02241_C165 | KIF23 Kinesin-like protein KIF23 | 5695 |
| Q02241_C412 | KIF23 Kinesin-like protein KIF23 | 5696 |
| P41229_C1047 | KDM5C Lysine-specific demethylase | 5697 |
| | 5C | |
| Q92538_C158 | GBF1 Golgi-specific brefeldin A- | 5698 |
| | resistance guanine nucleotide exchange | |
| | factor 1 | |
| Q9UEY8_C73 | ADD3 Gamma-adducin | 5699 |
| Q70J99_C448 | UNC13D Protein unc-13 homolog D | 5700 |
| P20339_C63 | RAB5A Ras-related protein Rab-5A | 5701 |
| Q9NZ08_C498 | ERAP1 Endoplasmic reticulum | 5702 |
| | aminopeptidase 1 | |
| Q9NZ09_C45 | UBAP1 Ubiquitin-associated protein 1 | 5703 |
| Q96IQ9_C75 | ZNF414 Zinc finger protein 414 | 5704 |
| Q96IQ9_C134 | ZNF414 Zinc finger protein 414 | 5705 |
| P55039_C270 | DRG2 Developmentally-regulated | 5706 |
| | GTP-binding protein 2 | |
| Q9H4A4_C254 | RNPEP Aminopeptidase B | 5707 |
| P15170_C453 | GSPT1 Eukaryotic peptide chain | 5708 |
| | release factor GTP-binding subunit | |
| | ERF3A | |
| O75152_C588 | ZC3H11A Zinc finger CCCH domain- | 5709 |
| | containing protein 11A | |
| Q9BWD1_C158 | ACAT2 Acetyl-CoA acetyltransferase, | 5710 |
| | cytosolic | |
| Q9UJX4_C203 | ANAPC5 Anaphase-promoting | 5711 |
| | complex subunit 5 | |
| Q96E11_C154 | MRRF Ribosome-recycling factor, | 5712 |
| | mitochondrial | |
| Q16666_C679 | IFI16 Gamma-interferon-inducible | 5713 |
| | protein 16 | |
| O00622_C100 | CYR61 Protein CYR61 | 5714 |
| O00629_C417 | KPNA4 Importin subunit alpha-4 | 5715 |
| P40227_C282 | CCT6A T-complex protein 1 subunit | 5716 |
| | zeta | |
| Q12830_C2697 | BPTF Nucleosome-remodeling factor | 5717 |
| | subunit BPTF | |
| P48960_C513 | CD97 CD97 antigen | 5718 |
| Q9NRZ9_C836 | HELLS Lymphoid-specific helicase | 5719 |
| P02765_C230 | AHSG Alpha-2-HS-glycoprotein | 5720 |
| Q9NTZ6_C545 | RBM12 RNA-binding protein 12 | 5721 |
| P20618_C89 | PSMB1 Proteasome subunit beta type-1 | 5722 |
| Q15365_C118 | PCBP1 Poly(rC)-binding protein 1 | 5723 |
| Q9BVL4_C629 | SELO Selenoprotein O | 5724 |
| B5ME19_C79 | EIF3CL Eukaryotic translation | 5725 |
| | initiation factor 3 subunit | |
| Q13905_C817 | RAPGEF1 Rap guanine nucleotide | 5726 |
| | exchange factor 1 | |
| P29401_C151 | TKT Transketolase | 5727 |
| P29401_C206 | TKT Transketolase | 5728 |
| P35244_C81 | RPA3 Replication protein A 14 kDa | 5729 |
| | subunit | |
| Q8WUB8_C57 | PHF10 PHD finger protein 10 | 5730 |
| P49711_C155 | CTCF Transcriptional repressor CTCF | 5731 |
| Q9UKL0_C35 | RCOR1 REST corepressor 1 | 5732 |
| Q92835_C506 | INPP5D Phosphatidylinositol 3,4,5- | 5733 |
| | trisphosphate 5-phosphase 1 | |
| Q9H3M7_C384 | TXNIP Thioredoxin-interacting protein | 5734 |
| Q92598_C650 | HSPH1 Heat shock protein 105 kDa | 5735 |
| Q09161_C36 | NCBP1 Nuclear cap-binding protein | 5736 |
| | subunit 1 | |
| Q99081_C129 | TCF12 Transcription factor 12 | 5737 |
| Q9Y4G6_C810 | TLN2 Talin-2 | 5738 |
| Q14566_C637 | MCM6 DNA replication licensing | 5739 |
| | factor MCM6 | |
| Q8NFQ8_C468 | TOR1AIP2 Torsin-1A-interacting | 5740 |
| | protein 2 | |
| Q15185_C20 | PTGES3 Prostaglandin E synthase 3 | 5741 |
| Q92619_C599 | HMHA1 Minor histocompatibility | 5742 |
| | protein HA-1 | |
| O94903_C15 | PROSC Proline synthase co-transcribed | 5743 |
| | bacterial homolog | |
| Q8N999_C294 | C12orf29 Uncharacterized protein | 5744 |
| | C12orf29 | |
| Q8NFC6_C2384 | BOD1L1 Biorientation of chromosomes | 5745 |
| | in cell division protein | |
| Q7L8L6_C689 | FASTKD5 FAST kinase domain- | 5746 |
| | containing protein 5 | |
| O43795_C779 | MYO1B Unconventional myosin-Ib | 5747 |
| Q9UPN3_C4388 | MACF1 Microtubule-actin cross- | 5748 |
| | linking factor 1, isoforms | |
| Q9UPN7_C455 | PPP6R1 Serine/threonine-protein | 5749 |
| | phosphatase 6 regulatory | |
| O75569_C106 | PRKRA Interferon-inducible double | 5750 |
| | stranded RNA-dependent | |
| Q9UPN9_C153 | TRIM33 E3 ubiquitin-protein ligase | 5751 |
| | TRIM33 | |
| Q9UPN9_C754 | TRIM33 E3 ubiquitin-protein ligase | 5752 |
| | TRIM33 | |
| Q9UPN9_{——}C786 | TRIM33 E3 ubiquitin-protein ligase | 5753 |
| | TRIM33 | |
| Q96HA1_C307 | POM121 Nuclear envelope pore | 5754 |
| | membrane protein POM 121 | |
| Q99460_C633 | PSMD1 26S proteasome non-ATPase | 5755 |
| | regulatory subunit 1 | |
| Q8WY36_C150 | BBX HMG box transcription factor | 5756 |
| | BBX | |
| Q8WY36_C712 | BBX HMG box transcription factor | 5757 |
| | BBX | |
| P40763_C468 | STAT3 Signal transducer and activator | 5758 |
| | of transcription 3 | |
| Q70E73_C847 | RAPH1 Ras-associated and pleckstrin | 5759 |
| | homology domains-containing protein 1 | |
| Q9BZQ8_C891 | FAM129A Protein Niban | 5760 |
| Q9NUU7_C224 | DDX19A ATP-dependent RNA | 5761 |
| | helicase DDX19A | |
| Q96KG9_C503 | SCYL1 N-terminal kinase-like protein | 5762 |
| Q96RU3_C248 | FNBP1 Formin-binding protein 1 | 5763 |
| Q14966_C1023 | ZNF638 Zinc finger protein 638 | 5764 |
| Q96FZ5_C12 | CMTM7 CKLF-like MARVEL | 5765 |
| | transmembrane domain-containing | |
| | protein 7 | |
| Q96FZ2_C97 | C3orf37 UPF0361 protein C3orf37 | 5766 |
| P49770_C194 | EIF2B2 Translation initiation factor | 5767 |
| | eIF-2B subunit beta | |
| P40306_C215 | PSMB10 Proteasome subunit beta type- | 5768 |
| | 10 | |
| O95639_C41 | CPSF4 Cleavage and polyadenylation | 5769 |
| | specificity factor subunit | |
| P25208_C89 | NFYB Nuclear transcription factor Y | 5770 |
| | subunit beta | |
| Q9BZ29_C628 | DOCK9 Dedicator of cytokinesis | 5771 |
| | protein 9 | |
| Q92688_C114 | ANP32B Acidic leucine-rich nuclear | 5772 |
| | phosphoprotein 32 family member B | |
| Q9H6R7_C507 | C2orf44 WD repeat-containing protein | 5773 |
| | C2orf44 | |
| O15067_C1044 | PFAS | 5774 |
| | Phosphoribosylformylglycinamidine | |
| | synthase | |
| P60891_C60 | PRPS1 Ribose-phosphate | 5775 |
| | pyrophosphokinase 1 | |
| Q86WN1_C449 | FCHSD1 FCH and double SH3 | 5776 |
| | domains protein 1 | |
| Q8TAA5_C97 | GRPEL2 GrpE protein homolog 2, | 5777 |
| | mitochondrial | |
| Q9NW64_C48 | RBM22 Pre-mRNA-splicing factor | 5778 |
| | RBM22 | |
| Q7Z4H7_C743 | HAUS6 HAUS augmin-like complex | 5779 |
| | subunit 6 | |
| Q13823_C336 | GNL2 Nucleolar GTP-binding protein 2 | 5780 |
| P49790_C148 | NUP153 Nuclear pore complex protein | 5781 |
| | Nup153 | |
| Q01813_C664 | PFKP 6-phosphofructokinase type C | 5782 |
| P49792_C503 | RANBP2 E3 SUMO-protein ligase | 5783 |
| | RanBP2 | |
| P49792_C3040 | RANBP2 E3 SUMO-protein ligase | 5784 |
| | RanBP2 | |
| Q00341_C104 | HDLBP Vigilin | 5785 |
| Q00610_C39 | CLTC Clathrin heavy chain 1 | 5786 |
| Q00610_C459 | CLTC Clathrin heavy chain 1 | 5787 |
| Q00610_C562 | CLTC Clathrin heavy chain 1 | 5788 |
| Q5SRE5_C585 | NUP188 Nucleoporin NUP188 | 5789 |
| | homolog | |
| Q9NVC6_C15 | MED17 Mediator of RNA polymerase | 5790 |
| | II transcription subunit | |
| Q9NVC6_C110 | MED17 Mediator of RNA polymerase | 5791 |
| | II transcription subunit | |
| Q96T76_C599 | MMS19 MMS19 nucleotide excision | 5792 |
| | repair protein homolog | |
| Q9Y618_C1297 | NCOR2 Nuclear receptor corepressor 2 | 5793 |
| Q9Y618_C2179 | NCOR2 Nuclear receptor corepressor 2 | 5794 |
| Q969T4_C145 | UBE2E3 Ubiquitin-conjugating enzyme | 5795 |
| | E2 E3 | |
| Q9Y613_C936 | FHOD1 FH1/FH2 domain-containing | 5796 |
| | protein 1 | |
| P09543_C111 | CNP 2,3-cyclic-nucleotide 3- | 5797 |
| | phosphodiesterase | |
| P32929_C252 | CTH Cystathionine gamma-lyase | 5798 |
| O75427_C283 | LRCH4 Leucine-rich repeat and | 5799 |
| | calponin homology domain-containing | |
| | 4 | |
| P07339_C110 | CTSD Cathepsin D | 5800 |
| Q92734_C80 | TFG Protein TFG | 5801 |
| Q00765_C118 | REEP5 Receptor expression-enhancing | 5802 |
| | protein 5 | |
| Q9Y4P8_C328 | WIPI2 WD repeat domain | 5803 |
| | phosphoinositide-interacting protein 2 | |
| Q4V328_C243 | GRIPAP1 GRIP1-associated protein 1 | 5804 |
| Q86UX6_C425 | STK32C Serine/threonine-protein | 5805 |
| | kinase 32C | |
| P43155_C169 | CRAT Carnitine O-acetyltransferase | 5806 |
| Q9BSJ8_C522 | ESYT1 Extended synaptotagmin-1 | 5807 |
| P35659_C161 | DEK Protein DEK | 5808 |
| P27695_C208 | APEX1 DNA-(apurinic or apyrimidinic | 5809 |
| | site) lyase | |
| P46013_C715 | MKI67 Antigen KI-67 | 5810 |
| P46013_C773 | MKI67 Antigen KI-67 | 5811 |
| Q9UJ70_C217 | NAGK N-acetyl-D-glucosamine kinase | 5812 |
| Q7Z5J4_C322 | RAI1 Retinoic acid-induced protein 1 | 5813 |
| Q9NRG7_C78 | SDR39U1 Epimerase family protein | 5814 |
| | SDR39U1 | |
| Q8WXH0_C6161 | SYNE2 Nesprin-2 | 5815 |
| Q8TC07_C686 | TBC1D15 TBC1 domain family | 5816 |
| | member 15 | |
| Q8N4N3_C254 | KLHL36 Kelch-like protein 36 | 5817 |
| Q99459_C769 | CDC5L Cell division cycle 5-like | 5818 |
| | protein | |
| P11216_C143 | PYGB Glycogen phosphorylase, brain | 5819 |
| | form | |
| Q6WCQ1_C831 | MPRIP Myosin phosphatase Rho- | 5820 |
| | interacting protein | |
| Q2TAZ0_C1458 | ATG2A Autophagy-related protein 2 | 5821 |
| | homolog A | |
| O43324_C147 | EEF1E1 Eukaryotic translation | 5822 |
| | elongation factor 1 epsilon | |
| Q96MF7_C140 | NSMCE2 E3 SUMO-protein ligase | 5823 |
| | NSE2 | |
| Q14653_C222 | IRF3 Interferon regulatory factor 3 | 5824 |
| Q15428_C56 | SF3A2 Splicing factor 3A subunit 2 | 5825 |
| Q9UJU2_C321 | LEF1 Lymphoid enhancer-binding | 5826 |
| | factor 1 | |
| P60953_C18 | CDC42 Cell division control protein 42 | 5827 |
| | homolog | |
| P60953_C81 | CDC42 Cell division control protein 42 | 5828 |
| | homolog | |
| Q9NYZ3_C539 | GTSE1 G2 and S phase-expressed | 5829 |
| | protein 1 | |
| Q9UHY7_C202 | ENOPH1 Enolase-phosphatase E1 | 5830 |
| O94768_C190 | STK17B Serine/threonine-protein | 5831 |
| | kinase 17B | |
| O15530_C21 | PDPK1 3-phosphoinositide-dependent | 5832 |
| | protein kinase 1 | |
| P21333_C810 | FLNA Filamin-A | 5833 |
| P21333_C1723 | FLNA Filamin-A | 5834 |
| P21333_C1865 | FLNA Filamin-A | 5835 |
| P21333_C2107 | FLNA Filamin-A | 5836 |
| Q9BWH6_C84 | RPAP1 RNA polymerase II-associated | 5837 |
| | protein 1 | |
| P51610_C292 | HCFC1 Host cell factor 1 | 5838 |
| P51610_C298 | HCFC1 Host cell factor 1 | 5839 |
| P51610_C1187 | HCFC1 Host cell factor 1 | 5840 |
| Q99683_C928 | MAP3K5 Mitogen-activated protein | 5841 |
| | kinase kinase kinase 5 | |
| Q9BWM7_C193 | SFXN3 Sideroflexin-3 | 5842 |
| Q96EM0_C205 | L3HYPDH Trans-L-3-hydroxyproline | 5843 |
| | dehydratase | |
| Q96SZ5_C258 | ADO 2-aminoethanethiol dioxygenase | 5844 |
| Q93100_C348 | PHKB Phosphorylase b kinase | 5845 |
| | regulatory subunit beta | |
| O75410_C715 | TACC1 Transforming acidic coiled- | 5846 |
| | coil-containing protein | |
| P35711_C492 | SOX5 Transcription factor SOX-5 | 5847 |
| Q9BXW9_C607 | FANCD2 Fanconi anemia group D2 | 5848 |
| | protein | |
| Q9Y4W2_C456 | LAS1L Ribosomal biogenesis protein | 5849 |
| | LAS1L | |
| P27635_C80 | RPL10 60S ribosomal protein L10 | 5850 |
| O95294_C611 | RASAL1 RasGAP-activating-like | 5851 |
| | protein 1 | |
| Q9Y4E6_C1103 | WDR7 WD repeat-containing protein 7 | 5852 |
| Q92616_C939 | GCN1L1 Translational activator GCN1 | 5853 |
| Q92615_C633 | LARP4B La-related protein 4B | 5854 |
| Q99661_C455 | KIF2C Kinesin-like protein KIF2C | 5855 |
| Q9Y4E8_C264 | USP15 Ubiquitin carboxyl-terminal | 5856 |
| | hydrolase 15 | |
| P24468_C213 | NR2F2 COUP transcription factor 2 | 5857 |
| P43490_C39 | NAMPT Nicotinamide | 5858 |
| | phosphoribosyltransferase | |
| Q8TE73_C4621 | DNAH5 Dynein heavy chain 5, | 5859 |
| | axonemal | |
| Q99996_C3868 | AKAP9 A-kinase anchor protein 9 | 5860 |
| P46060_C141 | RANGAP1 Ran GTPase-activating | 5861 |
| | protein 1 | |
| Q8WV28_C343 | BLNK B-cell linker protein | 5862 |
| Q8WV24_C242 | PHLDA1 Pleckstrin homology-like | 5863 |
| | domain family A member 1 | |
| P49327_C313 | FASN Fatty acid synthase | 5864 |
| P49327_C1881 | FASN Fatty acid synthase | 5865 |
| O75348_C104 | ATP6V1G1 V-type proton ATPase | 5866 |
| | subunit G 1 | |
| Q9UDR5_C342 | AASS Alpha-aminoadipic | 5867 |
| | semialdehyde synthase, mitochondrial | |
| Q13363_C312 | CTBP1 C-terminal-binding protein 1 | 5868 |
| O14929_C376 | HAT1 Histone acetyltransferase type B | 5869 |
| | catalytic subunit | |
| O14920_C464 | IKBKB Inhibitor of nuclear factor | 5870 |
| | kappa-B kinase subunit | |
| Q9NYK6_C73 | EURL Protein EURL homolog | 5871 |
| Q99536_C50 | VAT1 Synaptic vesicle membrane | 5872 |
| | protein VAT-1 homolog | |
| Q8N1G2_C9 | FTSJD2 Cap-specific mRNA | 5873 |
| | (nucleoside-2-O-)-methyltransfe | |
| Q96ME1_C459 | FBXL18 F-box/LRR-repeat protein 18 | 5874 |
| Q9BSQ5_C170 | CCM2 Malcavernin | 5875 |
| Q00688_C133 | FKBP3 Peptidyl-prolyl cis-trans | 5876 |
| | isomerase FKBP3 | |
| Q96FX7_C217 | TRMT61A tRNA (adenine(58)-N(1))- | 5877 |
| | methyltransferase catalytic subunit | |
| Q15691_C45 | MAPRE1 Microtubule-associated | 5878 |
| | protein RP/EB family member | |
| Q9NS87_C576 | KIF15 Kinesin-like protein KIF15 | 5879 |
| P13489_C220 | RNH1 Ribonuclease inhibitor | 5880 |
| Q9BPY3_C319 | FAM118B Protein FAM118B | 5881 |
| Q63HN8_C4348 | RNF213 E3 ubiquitin-protein ligase | 5882 |
| | RNF213 | |
| Q14839_C1018 | CHD4 Chromodomain-helicase-DNA- | 5883 |
| | binding protein 4 | |
| Q14839_C1019 | CHD4 Chromodomain-helicase-DNA- | 5884 |
| | binding protein 4 | |
| Q14839_C1121 | CHD4 Chromodomain-helicase-DNA- | 5885 |
| | binding protein 4 | |
| Q9HD33_C87 | MRPL47 39S ribosomal protein L47, | 5886 |
| | mitochondrial | |
| O14745_C206 | SLC9A3R1 Na(+)/H(+) exchange | 5887 |
| | regulatory cofactor NHE-RF1 | |
| O15287_C314 | FANCG Fanconi anemia group G | 5888 |
| | protein | |
| Q7L2J0_C429 | MEPCE 7SK snRNA methylphosphate | 5889 |
| | capping enzyme | |
| Q9BUK6_C561 | MSTO1 Protein misato homolog 1 | 5890 |
| Q9H5N1_C422 | RABEP2 Rab GTPase-binding effector | 5891 |
| | protein 2 | |
| Q7Z3K3_C1267 | POGZ Pogo transposable element with | 5892 |
| | ZNF domain | |
| Q96EN8_C558 | MOCOS Molybdenum cofactor | 5893 |
| | sulfurase | |
| P18074_C491 | ERCC2 TFIIH basal transcription factor | 5894 |
| | complex helicase | |
| Q9UKU7_C251 | ACAD8 Isobutyryl-CoA | 5895 |
| | dehydrogenase, mitochondrial | |
| Q96IY1_C175 | NSL1 Kinetochore-associated protein | 5896 |
| | NSL1 homolog | |
| Q06136_C121 | KDSR 3-ketodihydrosphingosine | 5897 |
| | reductase | |
| O75947_C101 | ATP5H ATP synthase subunit d, | 5898 |
| | mitochondrial | |
| P14921_C99 | ETS1 Protein C-ets-1 | 5899 |
| Q9NRY4_C924 | ARHGAP35 Rho GTPase-activating | 5900 |
| | protein 35 | |
| O60832_C74 | DKC1 H/ACA ribonucleoprotein | 5901 |
| | complex subunit 4 | |
| O95352_C524 | ATG7 Ubiquitin-like modifier- | 5902 |
| | activating enzyme ATG7 | |
| O95359_C2573 | TACC2 Transforming acidic coiled- | 5903 |
| | coil-containing protein | |
| Q6ZT12_C1858 | UBR3 E3 ubiquitin-protein ligase | 5904 |
| | UBR3 | |
| Q92990_C537 | GLMN Glomulin | 5905 |
| Q9Y383_C193 | LUC7L2 Putative RNA-binding protein | 5906 |
| | Luc7-like 2 | |
| O75400_C39 | PRPF40A Pre-mRNA-processing factor | 5907 |
| | 40 homolog A | |
| P00519_C1100 | ABL1 Tyrosine-protein kinase ABL1 | 5908 |
| P17676_C11 | CEBPB CCAAT/enhancer-binding | 5909 |
| | protein beta | |
| P53990_C125 | IST1 IST1 homolog | 5910 |
| P08729_C274 | KRT7 Keratin, type II cytoskeletal 7 | 5911 |
| O15357_C1187 | INPPL1 Phosphatidylinositol 3,4,5- | 5912 |
| | trisphosphate 5-phosphatase 2 | |
| Q8IX01_C970 | SUGP2 SURP and G-patch domain- | 5913 |
| | containing protein 2 | |
| Q9Y4D8_C1488 | HECTD4 Probable E3 ubiquitin-protein | 5914 |
| | ligase HECTD4 | |
| O60711_C376 | LPXN Leupaxin | 5915 |
| Q96IV0_C197 | NGLY1 Peptide-N(4)-(N-acetyl-beta- | 5916 |
| | glucosaminyl)asparagin | |
| P35637_C428 | FUS RNA-binding protein FUS | 5917 |
| Q6KC79_C1066 | NIPBL Nipped-B-like protein | 5918 |
| Q92973_C467 | TNPO1 Transportin-1 | 5919 |
| Q92973_C683 | TNPO1 Transportin-1 | 5920 |
| Q9BTE1_C160 | DCTN5 Dynactin subunit 5 | 5921 |
| P54577_C501 | YARS Tyrosine--tRNA ligase, | 5922 |
| | cytoplasmic | |
| O95218_C74 | ZRANB2 Zinc finger Ran-binding | 5923 |
| | domain-containing protein | |
| Q9NPI1_C367 | BRD7 Bromodomain-containing | 5924 |
| | protein 7 | |
| Q92576_C1771 | PHF3 PHD finger protein 3 | 5925 |
| Q92575_C186 | UBXN4 UBX domain-containing | 5926 |
| | protein 4 | |
| P24928_C13 | POLR2A DNA-directed RNA | 5927 |
| | polymerase II subunit RPB1 | |
| P24928_C451 | POLR2A DNA-directed RNA | 5928 |
| | polymerase II subunit RPB1 | |
| P24928_C1287 | POLR2A DNA-directed RNA | 5929 |
| | polymerase II subunit RPB1 | |
| Q8IVH4_C100 | MMAA Methylmalonic aciduria type A | 5930 |
| | protein, mitochondrial | |
| Q68E01_C596 | INTS3 Integrator complex subunit 3 | 5931 |
| Q15751_C2525 | HERC1 Probable E3 ubiquitin-protein | 5932 |
| | ligase HERC1 | |
| Q15751_C4811 | HERC1 Probable E3 ubiquitin-protein | 5933 |
| | ligase HERC1 | |
| Q8N680_C296 | ZBTB2 Zinc finger and BTB domain- | 5934 |
| | containing protein 2 | |
| Q13371_C35 | PDCL Phosducin-like protein | 5935 |
| Q9ULW3_C37 | ABT1 Activator of basal transcription 1 | 5936 |
| O95602_C613 | POLR1A DNA-directed RNA | 5937 |
| | polymerase I subunit RPA1 | |
| O95602_C1332 | POLR1A DNA-directed RNA | 5938 |
| | polymerase I subunit RPA1 | |
| O94776_C44 | MTA2 Metastasis-associated protein | 5939 |
| | MTA2 | |
| P61081_C111 | UBE2M NEDD8-conjugating enzyme | 5940 |
| | Ubc12 | |
| P85037_C665 | FOXK1 Forkhead box protein K1 | 5941 |
| P62263_C31 | RPS14 40S ribosomal protein S14 | 5942 |
| Q08945_C200 | SSRP1 FACT complex subunit SSRP1 | 5943 |
| Q9ULE6_C750 | PALD1 Paladin | 5944 |
| Q96F07_C223 | CYFIP2 Cytoplasmic FMR1-interacting | 5945 |
| | protein 2 | |
| Q15329_C121 | E2F5 Transcription factor E2F5 | 5946 |
| O95163_C213 | IKBKAP Elongator complex protein 1 | 5947 |
| Q6NXT1_C265 | ANKRD54 Ankyrin repeat domain- | 5948 |
| | containing protein 54 | |
| Q96EY5_C90 | FAM125A Multivesicular body subunit | 5949 |
| | 12A | |
| Q96CW6_C62 | SLC7A6OS Probable RNA polymerase | 5950 |
| | II nuclear localization protein | |
| Q7L1Q6_C349 | BZW1 Basic leucine zipper and W2 | 5951 |
| | domain-containing protein | |
| P15880_C188 | RPS2 40S ribosomal protein S2 | 5952 |
| Q5VT52_C88 | RPRD2 Regulation of nuclear pre- | 5953 |
| | mRNA domain-containing protein 2 | |
| Q9BZI7_C319 | UPF3B Regulator of nonsense | 5954 |
| | transcripts 3B | |
| Q9Y6K9_C131 | IKBKG NF-kappa-B essential | 5955 |
| | modulator | |
| P78527_C1507 | PRKDC DNA-dependent protein kinase | 5956 |
| | catalytic subunit | |
| P78527_C1791 | PRKDC DNA-dependent protein kinase | 5957 |
| | catalytic subunit | |
| P78537_C61 | BLOC1S1 Biogenesis of lysosome- | 5958 |
| | related organelles complex | |
| Q9Y6J0_C717 | CABIN1 Calcineurin-binding protein | 5959 |
| | cabin-1 | |
| Q15819_C69 | UBE2V2 Ubiquitin-conjugating | 5960 |
| | enzyme E2 variant 2 | |
| P52564_C38 | MAP2K6 Dual specificity mitogen- | 5961 |
| | activated protein kinase | |
| Q8N556_C684 | AFAP1 Actin filament-associated | 5962 |
| | protein 1 | |
| Q6P4F2_C151 | FDX1L Adrenodoxin-like protein, | 5963 |
| | mitochondrial | |
| O43598_C117 | RCL Deoxyribonucleoside 5- | 5964 |
| | monophosphate N-glycosidase | |
| P07996_C813 | THBS1 Thrombospondin-1 | 5965 |
| P07996_C946 | THBS1 Thrombospondin-1 | 5966 |
| P07996_C1167 | THBS1 Thrombospondin-1 | 5967 |
| P41182_C175 | BCL6 B-cell lymphoma 6 protein | 5968 |
| Q9BQC3_C251 | DPH2 Diphthamide biosynthesis | 5969 |
| | protein 2 | |
| O43572_C242 | AKAP10 A-kinase anchor protein 10, | 5970 |
| | mitochondrial | |
| Q96RQ3_C129 | MCCC1 Methylcrotonoyl-CoA | 5971 |
| | carboxylase subunit alpha, | |
| | mitochondrial | |
| Q96RQ3_C509 | MCCC1 Methylcrotonoyl-CoA | 5972 |
| | carboxylase subunit alpha, | |
| | mitochondrial | |
| Q9ULV4_C23 | CORO1C Coronin-1C | 5973 |
| Q7Z6M1_C29 | RABEPK Rab9 effector protein with | 5974 |
| | kelch motifs | |
| Q9NX38_C74 | FAM206A Protein FAM206A | 5975 |
| Q96HW7_C926 | INTS4 Integrator complex subunit 4 | 5976 |
| Q9HCD6_C431 | TANC2 Protein TANC2 | 5977 |
| Q13418_C239 | ILK Integrin-linked protein kinase | 5978 |
| P55036_C87 | PSMD4 26S proteasome non-ATPase | 5979 |
| | regulatory subunit 4 | |
| P62072_C29 | TIMM10 Mitochondrial import inner | 5980 |
| | membrane translocase subunit | |
| Q7Z2Z2_C474 | EFTUD1 Elongation factor Tu GTP- | 5981 |
| | binding domain-containing | |
| O00231_C257 | PSMD11 26S proteasome non-ATPase | 5982 |
| | regulatory subunit 11 | |
| P05023_C374 | ATP1A1 Sodium/potassium- | 5983 |
| | transporting ATPase subunit alpha | |
| P45985_C379 | MAP2K4 Dual specificity mitogen- | 5984 |
| | activated protein kinase | |
| P17029_C63 | ZKSCAN1 Zinc finger protein with | 5985 |
| | KRAB and SCAN domains 1 | |
| Q9BSW2_C341 | EFCAB4B EF-hand calcium-binding | 5986 |
| | domain-containing protein | |
| Q9P2R3_C34 | ANKFY1 Ankyrin repeat and FYVE | 5987 |
| | domain-containing protein | |
| Q9P2R3_C716 | ANKFY1 Ankyrin repeat and FYVE | 5988 |
| | domain-containing protein | |
| P31040_C536 | SDHA Succinate dehydrogenase | 5989 |
| P09104_C357 | ENO2 Gamma-enolase | 5990 |
| O76075_C108 | DFFB DNA fragmentation factor | 5991 |
| | subunit beta | |
| Q9P2D3_C1546 | HEATR5B HEAT repeat-containing | 5992 |
| | protein 5B | |
| Q9P2D6_C575 | FAM135A Protein FAM135A | 5993 |
| Q15334_C697 | LLGL1 Lethal(2) giant larvae protein | 5994 |
| | homolog 1 | |
| P17735_C332 | TAT Tyrosine aminotransferase | 5995 |
| Q9NXW9_C159 | ALKBH4 Probable alpha-ketoglutarate- | 5996 |
| | dependent dioxygenase | |
| Q5BKX5_C271 | C19orf54 UPF0692 protein C19orf54 | 5997 |
| Q92888_C752 | ARHGEF1 Rho guanine nucleotide | 5998 |
| | exchange factor 1 | |
| Q96EV8_C302 | DTNBP1 Dysbindin | 5999 |
| A6NC98_C1382 | CCDC88B Coiled-coil domain- | 6000 |
| | containing protein 88B | |
| Q9UQR0_C324 | SCML2 Sex comb on midleg-like | 6001 |
| | protein 2 | |
| P04083_C263 | ANXA1 Annexin A1 | 6002 |
| Q3V6T2_C1729 | CCDC88A Girdin | 6003 |
| Q12906_C278 | ILF3 Interleukin enhancer-binding | 6004 |
| | factor 3 | |
| Q14315_C2555 | FLNC Filamin-C | 6005 |
| Q14315_C2660 | FLNC Filamin-C | 6006 |
| O43813_C108 | LANCL1 LanC-like protein 1 | 6007 |
| Q8IY67_C297 | RAVER1 Ribonucleoprotein PTB- | 6008 |
| | binding 1 | |
| O43818_C416 | RRP9 U3 small nucleolar RNA- | 6009 |
| | interacting protein 2 | |
| Q9UI43_C126 | FTSJ2 Putative ribosomal RNA | 6010 |
| | methyltransferase 2 | |
| P04150_C287 | NR3C1 Glucocorticoid receptor | 6011 |
| Q96JH7_C897 | VCPIP1 Deubiquitinating protein | 6012 |
| | VCIP135 | |
| Q9UJA5_C243 | TRMT6 tRNA (adenine(58)-N(1))- | 6013 |
| | methyltransferase non-catalytic subunit | |
| | TRM6 | |
| Q9UJA5_C492 | TRMT6 tRNA (adenine(58)-N(1))- | 6014 |
| | methyltransferase non-catalytic subunit | |
| | TRM6 | |
| Q6PJG6_C820 | BRAT1 BRCA1-associated ATM | 6015 |
| | activator 1 | |
| Q7LBC6_C509 | KDM3B Lysine-specific demethylase | 6016 |
| | 3B | |
| P62140_C104 | PPP1CB Serine/threonine-protein | 6017 |
| | phosphatase PP1-beta catalytic subunit | |
| | beta | |
| P62140_C244 | PPP1CB Serine/threonine-protein | 6018 |
| | phosphatase PP1-beta catalytic subunit | |
| | beta | |
| Q5JPH6_C314 | EARS2 Probable glutamate--tRNA | 6019 |
| | ligase, mitochondrial | |
| P12429_C246 | ANXA3 Annexin A3 | 6020 |
| Q5D0E6_C468 | DALRD3 DALR anticodon-binding | 6021 |
| | domain-containing protein 3 | |
| Q13315_C2991 | ATM Serine-protein kinase ATM | 6022 |
| P51116_C109 | FXR2 Fragile X mental retardation | 6023 |
| | syndrome-related protein | |
| Q14683_C1210 | SMC1A Structural maintenance of | 6024 |
| | chromosomes protein 1A | |
| P11766_C268 | ADH5 Alcohol dehydrogenase class-3 | 6025 |
| P11766_C282 | ADH5 Alcohol dehydrogenase class-3 | 6026 |
| Q8NI08_C302 | NCOA7 Nuclear receptor coactivator 7 | 6027 |
| Q9HD64_C33 | XAGE1E G antigen family D member | 6028 |
| | 2 | |
| Q15796_C312 | SMAD2 Mothers against | 6029 |
| | decapentaplegic homolog 2 | |
| Q9UBC1_C304 | NFKBIL1 NF-kappa-B inhibitor-like | 6030 |
| | protein 1 | |
| Q9HB65_C15 | ELL3 RNA polymerase II elongation | 6031 |
| | factor ELL3 | |
| Q9HB65_C102 | ELL3 RNA polymerase II elongation | 6032 |
| | factor ELL3 | |
| Q9NUL3_C491 | STAU2 Double-stranded RNA-binding | 6033 |
| | protein Staufen homolo | |
| Q9BYI3_C300 | FAM126A Hyccin | 6034 |
| P51149_C143 | RAB7A Ras-related protein Rab-7a | 6035 |
| P09110_C26 | ACAA1 3-ketoacyl-CoA thiolase, | 6036 |
| | peroxisomal | |
| Q9UL15_C360 | BAG5 BAG family molecular | 6037 |
| | chaperone regulator 5 | |
| P55263_C160 | ADK Adenosine kinase | 6038 |
| Q9UL12_C799 | SARDH Sarcosine dehydrogenase, | 6039 |
| | mitochondrial | |
| O15061_C1225 | SYNM Synemin | 6040 |
| Q9NP64_C52 | ZCCHC17 Nucleolar protein of 40 kDa | 6041 |
| Q96S59_C527 | RANBP9 Ran-binding protein 9 | 6042 |
| O15382_C195 | BCAT2 Branched-chain-amino-acid | 6043 |
| | aminotransferase, mitochondrial | |
| O15382_C345 | BCAT2 Branched-chain-amino-acid | 6044 |
| | aminotransferase, mitoch | |
| P45974_C838 | USP5 Ubiquitin carboxyl-terminal | 6045 |
| | hydrolase 5 | |
| O14497_C1874 | ARID1A AT-rich interactive domain- | 6046 |
| | containing protein 1A | |
| O75521_C368 | ECI2 Enoyl-CoA delta isomerase 2, | 6047 |
| | mitochondrial | |
| Q92769_C111 | HDAC2 Histone deacetylase 2 | 6048 |
| Q96AE4_C148 | FUBP1 Far upstream element-binding | 6049 |
| | protein 1 | |
| Q8NFI3_C454 | ENGASE Cytosolic endo-beta-N- | 6050 |
| | acetylglucosaminidase | |
| Q9P107_C336 | GMIP GEM-interacting protein | 6051 |
| Q01469_C47 | FABP5 Fatty acid-binding protein, | 6052 |
| | epidermal | |
| Q6PCE3_C303 | PGM2L1 Glucose 1,6-bisphosphate | 6053 |
| | synthase | |
| Q9HAV0_C204 | GNB4 Guanine nucleotide-binding | 6054 |
| | protein subunit beta-4 | |
| Q9Y3C8_C69 | UFC1 Ubiquitin-fold modifier- | 6055 |
| | conjugating enzyme 1 | |
| Q8N806_C374 | UBR7 Putative E3 ubiquitin-protein | 6056 |
| | ligase UBR7 | |
| O43172_C299 | PRPF4 U4/U6 small nuclear | 6057 |
| | ribonucleoprotein Prp4 | |
| O94851_C552 | MICAL2 Protein-methionine sulfoxide | 6058 |
| | oxidase MICAL2 | |
| O60716_C692 | CTNND1 Catenin delta-1 | 6059 |
| O94855_C1022 | SEC24D Protein transport protein | 6060 |
| | Sec24D | |
| Q9BQS8_C1062 | FYCO1 FYVE and coiled-coil domain- | 6061 |
| | containing protein 1 | |
| Q9BSC4_C16 | NOL10 Nucleolar protein 10 | 6062 |
| Q9H5X1_C90 | FAM96A MIP18 family protein | 6063 |
| | FAM96A | |
| Q13325_C429 | IFIT5 Interferon-induced protein with | 6064 |
| | tetratricopeptide | |
| Q13325_C476 | IFIT5 Interferon-induced protein with | 6065 |
| | tetratricopeptide | |
| P23526_C228 | AHCY Adenosylhomocysteinase | 6066 |
| Q9UG63_C586 | ABCF2 ATP-binding cassette sub- | 6067 |
| | family F member 2 | |
| Q9BQA1_C26 | WDR77 Methylosome protein 50 | 6068 |
| Q9BQA1_C172 | WDR77 Methylosome protein 50 | 6069 |
| Q9BQA1_C208 | WDR77 Methylosome protein 50 | 6070 |
| Q9UKJ3_C569 | GPATCH8 G patch domain-containing | 6071 |
| | protein 8 | |
| Q15654_C47 | TRIP6 Thyroid receptor-interacting | 6072 |
| | protein 6 | |
| P36873_C202 | PPP1CC Serine/threonine-protein | 6073 |
| | phosphatase PP1-gamma cat | |
| Q8WXF1_C216 | PSPC1 Paraspeckle component 1 | 6074 |
| Q13555_C273 | CAMK2G Calcium/calmodulin- | 6075 |
| | dependent protein kinase type I | |
| P55265_C622 | ADAR Double-stranded RNA-specific | 6076 |
| | adenosine deaminase | |
| P60900_C115 | PSMA6 Proteasome subunit alpha type- | 6077 |
| | 6 | |
| P50336_C183 | PPOX Protoporphyrinogen oxidase | 6078 |
| Q63ZY3_C88 | KANK2 KN motif and ankyrin repeat | 6079 |
| | domain-containing prot | |
| Q9HB71_C173 | CACYBP Calcyclin-binding protein | 6080 |
| Q9P2T1_C224 | GMPR2 GMP reductase 2 | 6081 |
| Q5VIR6_C365 | VPS53 Vacuolar protein sorting- | 6082 |
| | associated protein 53 homolog | |
| P51003_C677 | PAPOLA Poly(A) polymerase alpha | 6083 |
| Q9NVE5_C50 | USP40 Ubiquitin carboxyl-terminal | 6084 |
| | hydrolase 40 | |
| | R.NQGGTC*YLNSLLQTLHFTPEFR.E | |
| Q9Y2Z2_C315 | MTO1 Protein MTO1 homolog, | 6085 |
| | mitochondrial | |
| P30042_C52 | C21orf33 ES1 protein homolog, | 6086 |
| | mitochondrial | |
| Q96CX6_C170 | LRRC58 Leucine-rich repeat- | 6087 |
| | containing protein 58 | |
| Q96CX2_C50 | KCTD12 BTB/POZ domain-containing | 6088 |
| | protein KCTD12 | |
| P18887_C20 | XRCC1 DNA repair protein XRCC1 | 6089 |
| Q9C0C9_C1288 | UBE2O Ubiquitin-conjugating enzyme | 6090 |
| | E2 O | |
| O00762_C114 | UBE2C Ubiquitin-conjugating enzyme | 6091 |
| | E2 C | |
| Q9Y520_C481 | PRRC2C Protein PRRC2C | 6092 |
| Q9Y520_C953 | PRRC2C Protein PRRC2C | 6093 |
| O75828_C227 | CBR3 Carbonyl reductase | 6094 |
| O75534_C42 | CSDE1 Cold shock domain-containing | 6095 |
| | protein E1 | |
| P09651_C175 | HNRNPA1 Heterogeneous nuclear | 6096 |
| | ribonucleoprotein A1 | |
| Q9H7T3_C143 | C10orf95 Uncharacterized protein | 6097 |
| | C10orf95 | |
| Q96R06_C382 | SPAG5 Sperm-associated antigen 5 | 6098 |
| O43837_C185 | IDH3B Isocitrate dehydrogenase | 6099 |
| Q96AD5_C312 | PNPLA2 Patatin-like phospholipase | 6100 |
| | domain-containing prote | |
| Q96BD8_C142 | SKA1 Spindle and kinetochore- | 6101 |
| | associated protein 1 | |
| Q8NFH4_C311 | NUP37 Nucleoporin Nup37 | 6102 |
| Q9NRA8_C767 | EIF4ENIF1 Eukaryotic translation | 6103 |
| | initiation factor 4E transp | |
| Q9NTM9_C248 | CUTC Copper homeostasis protein | 6104 |
| | cutC homolog | |
| Q8TEU7_C1491 | RAPGEF6 Rap guanine nucleotide | 6105 |
| | exchange factor 6 | |
| Q14207_C842 | NPAT Protein NPAT | 6106 |
| Q14204_C1932 | DYNC1H1 Cytoplasmic dynein 1 | 6107 |
| | heavy chain 1 | |
| Q14204_C4510 | DYNC1H1 Cytoplasmic dynein 1 | 6108 |
| | heavy chain 1 | |
| O60684_C273 | KPNA6 Importin subunit alpha-7 | 6109 |
| Q92841_C447 | DDX17 Probable ATP-dependent RNA | 6110 |
| | helicase DDX17 | |
| P07814_C1497 | EPRS Bifunctional glutamate/proline-- | 6111 |
| | tRNA ligase | |
| P19174_C247 | PLCG1 1-phosphatidylinositol 4,5- | 6112 |
| | bisphosphate phosphodie | |
| P19174_C646 | PLCG1 1-phosphatidylinositol 4,5- | 6113 |
| | bisphosphate phosphodie | |
| P22102_C466 | GART Trifunctional purine | 6114 |
| | biosynthetic protein adenosin | |
| O75643_C238 | SNRNP200 U5 small nuclear | 6115 |
| | ribonucleoprotein 200 kDa helicas | |
| O75643_C576 | SNRNP200 U5 small nuclear | 6116 |
| | ribonucleoprotein 200 kDa helicas | |
| Q04759_C17 | PRKCQ Protein kinase C theta type | 6117 |
| O94992_C122 | HEXIM1 Protein HEXIM1 | 6118 |
| P61106_C26 | RAB14 Ras-related protein Rab-14 | 6119 |
| P50851_C982 | LRBA Lipopolysaccharide-responsive | 6120 |
| | and beige-like ancho | |
| P50851_C1228 | LRBA Lipopolysaccharide-responsive | 6121 |
| | and beige-like ancho | |
| Q6FI81_C237 | CIAPIN1 Anamorsin | 6122 |
| Q6FI81_C274 | CIAPIN1 Anamorsin | 6123 |
| Q6FI81_C285 | CIAPIN1 Anamorsin | 6124 |
| Q96RL1_C283 | UIMC1 BRCA1-A complex subunit | 6125 |
| | RAP80 | |
| O60264_C165 | SMARCA5 SWI/SNF-related matrix- | 6126 |
| | associated actin-dependent | |
| P11586_C152 | MTHFD1 C-1-tetrahydrofolate | 6127 |
| | synthase, cytoplasmic | |
| Q86Y56_C138 | HEATR2 HEAT repeat-containing | 6128 |
| | protein 2 | |
| Q15648_C681 | MED1 Mediator of RNA polymerase II | 6129 |
| | transcription subunit | |
| Q6P4I2_C106 | WDR73 WD repeat-containing protein | 6130 |
| | 73 | |
| P62318_C20 | SNRPD3 Small nuclear | 6131 |
| | ribonucleoprotein Sm D3 | |
| Q53HC9_C198 | TSSC1 Protein TSSC1 | 6132 |
| Q9BRF8_C143 | CPPED1 Calcineurin-like | 6133 |
| | phosphoesterase domain-containing | |
| O95630_C221 | STAMBP STAM-binding protein | 6134 |
| P30101_C92 | PDIA3 Protein disulfide-isomerase A3 | 6135 |
| P48200_C201 | IREB2 Iron-responsive element-binding | 6136 |
| | protein 2 | |
| O14730_C22 | RIOK3 Serine/threonine-protein kinase | 6137 |
| | RIO3 | |
| P09884_C306 | POLA1 DNA polymerase alpha | 6138 |
| | catalytic subunit | |
| P09884_C1224 | POLA1 DNA polymerase alpha | 6139 |
| | catalytic subunit | |
| P12034_C202 | FGF5 Fibroblast growth factor 5 | 6140 |
| P10768_C243 | ESD S-formylglutathione hydrolase | 6141 |
| Q7L2E3_C1183 | DHX30 Putative ATP-dependent RNA | 6142 |
| | helicase DHX30 | |
| Q9UJM3_C113 | ERRFI1 ERBB receptor feedback | 6143 |
| | inhibitor 1 | |
| Q9UJM3_C204 | ERRFI1 ERBB receptor feedback | 6144 |
| | inhibitor 1 | |
| Q8WTW3_C318 | COG1 Conserved oligomeric Golgi | 6145 |
| | complex subunit 1 | |
| Q96KP1_C638 | EXOC2 Exocyst complex component 2 | 6146 |
| P62136_C245 | PPP1CA Serine/threonine-protein | 6147 |
| | phosphatase PP1-alpha cat | |
| Q9NQR4_C44 | NIT2 Omega-amidase NIT2 | 6148 |
| P52948_C1711 | NUP98 Nuclear pore complex protein | 6149 |
| | Nup98-Nup96 | |
| O15014_C178 | ZNF609 Zinc finger protein 609 | 6150 |
| P13674_C167 | P4HA1 Prolyl 4-hydroxylase subunit | 6151 |
| | alpha-1 | |
| O95232_C43 | LUC7L3 Luc7-like protein 3 | 6152 |
| Q96JM3_C119 | CHAMP1 Chromosome alignment- | 6153 |
| | maintaining phosphoprotein 1 | |
| Q12857_C404 | NFIA Nuclear factor 1 A-type | 6154 |
| O75818_C49 | RPP40 Ribonuclease P protein subunit | 6155 |
| | p40 | |
| Q8WYJ6_C102 | SEPT1 Septin-1 | 6156 |
| O75815_C360 | BCAR3 Breast cancer anti-estrogen | 6157 |
| | resistance protein 3 | |
| Q08J23_C235 | NSUN2 tRNA (cytosine(34)-C(5))- | 6158 |
| | methyltransferase | |
| Q08J23_C758 | NSUN2 tRNA (cytosine(34)-C(5))- | 6159 |
| | methyltransferase | |
| Q9H2U1_C135 | DHX36 Probable ATP-dependent RNA | 6160 |
| | helicase DHX36 | |
| Q86VQ1_C51 | GLCCI1 Glucocorticoid-induced | 6161 |
| | transcript 1 protein | |
| Q04695_C336 | KRT17 Keratin, type I cytoskeletal 17 | 6162 |
| Q9Y530_C33 | C6orf130 O-acetyl-ADP-ribose | 6163 |
| | deacetylase C6orf130 | |
| Q9Y530_C122 | C6orf130 O-acetyl-ADP-ribose | 6164 |
| | deacetylase C6orf130 | |
| O43929_C16 | ORC4 Origin recognition complex | 6165 |
| | subunit 4 | |
| Q66K14_C289 | TBC1D9B TBC1 domain family | 6166 |
| | member 9B | |
| Q8N0W3_C484 | FUK L-fucose kinase | 6167 |
| Q8N0W3_C582 | FUK L-fucose kinase | 6168 |
| P61812_C309 | TGFB2 Transforming growth factor | 6169 |
| | beta-2 | |
| Q86X10_C923 | RALGAPB Ral GTPase-activating | 6170 |
| | protein subunit beta | |
| O95833_C219 | CLIC3 Chloride intracellular channel | 6171 |
| | protein 3 | |
| J3KR12_C95 | Uncharacterized protein | 6172 |
| P12931_C280 | SRC Proto-oncogene tyrosine-protein | 6173 |
| | kinase Src | |
| Q13535_C2336 | ATR Serine/threonine-protein kinase | 6174 |
| | ATR | |
| Q6PIW4_C200 | FIGNL1 Fidgetin-like protein 1 | 6175 |
| Q6PIW4_C672 | FIGNL1 Fidgetin-like protein 1 | 6176 |
| P31939_C575 | ATIC Bifunctional purine biosynthesis | 6177 |
| | protein PURH | |
| Q15291_C212 | RBBP5 Retinoblastoma-binding protein | 6178 |
| | 5 | |
| O14618_C227 | CCS Copper chaperone for superoxide | 6179 |
| | dismutase | |
| Q9BPW8_C106 | NIPSNAP1 Protein NipSnap homolog 1 | 6180 |
| Q9BPW8_C138 | NIPSNAP1 Protein NipSnap homolog 1 | 6181 |
| Q8IUC6_C192 | TICAM1 TIR domain-containing | 6182 |
| | adapter molecule 1 | |
| Q9P0V3_C787 | SH3BP4 SH3 domain-binding protein 4 | 6183 |
| P52888_C18 | THOP1 Thimet oligopeptidase | 6184 |
| Q9H6T3_C519 | RPAP3 RNA polymerase II-associated | 6185 |
| | protein 3 | |
| Q07157_C1727 | TJP1 Tight junction protein ZO-1 | 6186 |
| Q15061_C380 | WDR43 WD repeat-containing protein | 6187 |
| | 43 | |
| Q15067_C449 | ACOX1 Peroxisomal acyl-coenzyme A | 6188 |
| | oxidase 1 | |
| Q6P1X5_C1093 | TAF2 Transcription initiation factor | 6189 |
| | TFIID subunit 2 | |
| P67936_C154 | TPM4 Tropomyosin alpha-4 chain | 6190 |
| Q92918_C484 | MAP4K1 Mitogen-activated protein | 6191 |
| | kinase kinase kinase kin | |
| O15226_C37 | NKRF NF-kappa-B-repressing factor | 6192 |
| Q9P2J5_C527 | LARS Leucine--tRNA ligase, | 6193 |
| | cytoplasmic | |
| Q96GY3_C28 | LIN37 Protein lin-37 homolog | 6194 |
| Q96GY3_C176 | LIN37 Protein lin-37 homolog | 6195 |
| Q8TF72_C1104 | SHROOM3 Protein Shroom3 | 6196 |
| Q8TF72_C1367 | SHROOM3 Protein Shroom3 | 6197 |
| P62906_C164 | RPL10A 60S ribosomal protein L10a | 6198 |
| Q86VP6_C356 | CAND1 Cullin-associated NEDD8- | 6199 |
| | dissociated protein 1 | |
| Q92597_C49 | NDRG1 Protein NDRG1 | 6200 |
| P35237_C370 | SERPINB6 Serpin B6 | 6201 |
| P30876_C177 | POLR2B DNA-directed RNA | 6202 |
| | polymerase II subunit RPB2 | |
| P30876_C622 | POLR2B DNA-directed RNA | 6203 |
| | polymerase II subunit RPB2 | |
| P14324_C62 | FDPS Farnesyl pyrophosphate synthase | 6204 |
| Q9Y3F4_C152 | STRAP Serine-threonine kinase | 6205 |
| | receptor-associated protei | |
| P27144_C22 | AK4 Adenylate kinase isoenzyme 4, | 6206 |
| | mitochondrial | |
| Q14135_C167 | VGLL4 Transcription cofactor | 6207 |
| | vestigial-like protein 4 | |
| Q14135_C205 | VGLL4 Transcription cofactor | 6208 |
| | vestigial-like protein 4 | |
| O43143_C750 | DHX15 Putative pre-mRNA-splicing | 6209 |
| | factor ATP-dependent RN | |
| Q8IW35_C599 | CEP97 Centrosomal protein of 97 kDa | 6210 |
| P67775_C165 | PPP2CA Serine/threonine-protein | 6211 |
| | phosphatase 2A catalytic | |
| P67775_C196 | PPP2CA Serine/threonine-protein | 6212 |
| | phosphatase 2A catalytic | |
| Q92667_C64 | AKAP1 A-kinase anchor protein 1, | 6213 |
| | mitochondrial | |
| Q32MZ4_C726 | LKRFIP1 Leucine-rich repeat | 6214 |
| | flightless-interacting protein | |
| Q32MZ4_C750 | LRRFIP1 Leucine-rich repeat | 6215 |
| | flightless-interacting protein | |
| Q99615_C58 | DNAJC7 DnaJ homolog subfamily C | 6216 |
| | member 7 | |
| Q99615_C175 | DNAJC7 DnaJ homolog subfamily C | 6217 |
| | member 7 | |
| Q99615_C225 | DNAJC7 DnaJ homolog subfamily C | 6218 |
| | member 7 | |
| Q05086_C198 | UBE3A Ubiquitin-protein ligase E3A | 6219 |
| Q52LW3_C1215 | ARHGAP29 Rho GTPase-activating | 6220 |
| | protein 29 | |
| Q52LW3_C1239 | ARHGAP29 Rho GTPase-activating | 6221 |
| | protein 29 | |
| Q9UNS1_C700 | TIMELESS Protein timeless homolog | 6222 |
| O94885_C1120 | SASH1 SAM and SH3 domain- | 6223 |
| | containing protein 1 | |
| P10619_C281 | CTSA Lysosomal protective protein | 6224 |
| Q9UHQ1_C192 | NARF Nuclear prelamin A recognition | 6225 |
| | fector | |
| Q9P2N6_C459 | KANSL3 KAT8 regulatory NSL | 6226 |
| | complex subunit 3 | |
| Q9ULC5_C322 | ACSL5 Long-chain-fatty-acid--CoA | 6227 |
| | ligase 5 | |
| O75116_C428 | ROCK2 Rho-associated protein kinase | 6228 |
| | 2 | |
| P62330_C90 | ARF6 ADP-ribosylation factor 6 | 6229 |
| Q96G74_C434 | OTUD5 OTU domain-containing | 6230 |
| | protein 5 | |
| Q13444_C667 | ADAM15 Disintegrin and | 6231 |
| | metalloproteinase domain-containin | |
| Q5UIP0_C1904 | RIF1 Telomere-associated protein RIF1 | 6232 |
| Q9NZB2_C919 | FAM120A Constitutive coactivator of | 6233 |
| | PPAR-gamma-like protei | |
| O94805_C32 | ACTL6B Actin-like protein 6B | 6234 |
| Q9UDY8_C71 | MALT1 Mucosa-associated lymphoid | 6235 |
| | tissue lymphoma translo | |
| P06400_C590 | RB1 Retinoblastoma-associated protein | 6236 |
| P55199_C433 | ELL RNA polymerase II elongation | 6237 |
| | factor ELL | |
| P55199_C494 | ELL RNA polymerase II elongation | 6238 |
| | factor ELL | |
| Q96KR1_C525 | ZFR Zinc finger RNA-binding protein | 6239 |
| Q9H0K6_C640 | PUS7L Pseudouridylate synthase 7 | 6240 |
| | homolog-like protein | |
| Q96K76_C856 | USP47 Ubiquitin carboxyl-terminal | 6241 |
| | hydrolase 47 | |
| O14727_C115 | APAF1 Apoptotic protease-activating | 6242 |
| | factor 1 | |
| Q96N21_C52 | ENTHD2 AP-4 complex accessory | 6243 |
| | subunit tepsin | |
| Q9HC38_C171 | GLOD4 Glyoxalase domain-containing | 6244 |
| | protein 4 | |
| Q9HC38_C221 | GLOD4 Glyoxalase domain-containing | 6245 |
| | protein 4 | |
| Q9NSD9_C76 | FARSB Phenylalanine--tRNA ligase | 6246 |
| | beta subunit | |
| O75376_C1274 | NCOR1 Nuclear receptor corepressor 1 | 6247 |
| O75376_C2322 | NCOR1 Nuclear receptor corepressor 1 | 6248 |
| Q15058_C291 | KIF14 Kinesin-like protein KIF14 | 6249 |
| Q15050_C52 | RRS1 Ribosome biogenesis regulatory | 6250 |
| | protein homolog | |
| Q15052_C667 | ARHGEF6 Rho guanine nucleotide | 6251 |
| | exchange factor 6 | |
| O00592_C344 | PODXL Podocalyxin | 6252 |
| P49903_C317 | SEPHS1 Selenide, water dikinase 1 | 6253 |
| E7EVH7_C562 | KLC1 Kinesin light chain 1 | 6254 |
| Q9Y6A5_C242 | TACC3 Transforming acidic coiled- | 6255 |
| | coil-containing protein | |
| Q6P1K8_C299 | GTF2H2D General transcription factor | 6256 |
| | IIH subunit 2-like pr | |
| Q3B7T1_C589 | EDRF1 Erythroid differentiation- | 6257 |
| | related factor 1 | |
| Q9H0W8_C411 | SMG9 Protein SMG9 | 6258 |
| Q86XP3_C281 | DDX42 ATP-dependent RNA helicase | 6259 |
| | DDX42 | |
| Q08257_C45 | CRYZ Quinone oxidoreductase | 6260 |
| O75879_C170 | PET112 Glutamyl-tRNA(Gln) | 6261 |
| | amidotransferase subunit B, | |
| | mitochondrial | |
| Q92696_C354 | RABGGTA Geranylgeranyl transferase | 6262 |
| | type-2 subunit alpha | |
| Q15772_C1052 | SPEG Striated muscle preferentially | 6263 |
| | expressed protein k | |
| O75616_C348 | ERAL1 GTPase Era, mitochondrial | 6264 |
| Q6UB35_C291 | MTHFD1L Monofunctional C1- | 6265 |
| | tetrahydrofolate synthase, | |
| | mitochondrial | |
| Q8N392_C323 | ARHGAP18 Rho GTPase-activating | 6266 |
| | protein 18 | |
| Q9UHR6_C188 | ZNHIT2 Zinc finger HIT domain- | 6267 |
| | containing protein 2 | |
| P57081_C95 | WDR4 tRNA (guanine-N(7)-)- | 6268 |
| | methyltransferase subunit WDR | |
| Q9H4L4_C274 | SENP3 Sentrin-specific protease 3 | 6269 |
| Q14149_C632 | MORC3 MORC family CW-type zinc | 6270 |
| | finger protein 3 | |
| O95455_C175 | TGDS dTDP-D-glucose 4,6- | 6271 |
| | dehydratase | |
| O95453_C169 | PARN Poly(A)-specific ribonuclease | 6272 |
| | PARN | |
| Q99426_C216 | TBCB Tubulin-folding cofactor B | 6273 |
| Q16566_C202 | CAMK4 Calcium/calmodulin- | 6274 |
| | dependent protein kinase type I | |
| Q9Y5P6_C113 | GMPPB Mannose-1-phosphate | 6275 |
| | guanyltransferase beta | |
| Q9UBT2_C158 | UBA2 SUMO-activating enzyme | 6276 |
| | subunit 2 | |
| Q96C36_C225 | PYCR2 Pyrroline-5-carboxylate | 6277 |
| | reductase 2 | |
| Q6NS38_C192 | ALKBH2 Alpha-ketoglutarate- | 6278 |
| | dependent dioxygenase alkB homolog 2 | |
| Q86SQ0_C385 | PHLDB2 Pleckstrin homology-like | 6279 |
| | domain family B member 2 | |
| Q6DKI1_C184 | RPL7L1 60S ribosomal protein L7-like | 6280 |
| | 1 | |
| Q06203_C503 | PPAT Amidophosphoribosyltransferase | 6281 |
| Q7Z6I6_C965 | ARHGAP30 Rho GTPase-activating | 6282 |
| | protein 30 | |
| Q9ULP9_C223 | TBC1D24 TBC1 domain family | 6283 |
| | member 24 | |
| P34897_C343 | SHMT2 Serine | 6284 |
| | hydroxymethyltransferase, | |
| | mitochondrial | |
| P49736_C315 | MCM2 DNA replication licensing | 6285 |
| | factor MCM2 | |
| O95071_C2094 | UBR5 E3 ubiquitin-protein ligase | 6286 |
| | UBR5 | |
| O95071_C2173 | UBR5 E3 ubiquitin-protein ligase | 6287 |
| | UBR5 | |
| Q96L91_C162 | EP400 E1A-binding protein p400 | 6288 |
| Q6XZF7_C1434 | DNMBP Dynamin-binding protein | 6289 |
| P34949_C11 | MPI Mannose-6-phosphate isomerase | 6290 |
| Q15437_C180 | SEC23B Protein transport protein | 6291 |
| | Sec23B | |
| P09960_C543 | LTA4H Leukotriene A-4 hydrolase | 6292 |
| P30460_C349 | HLA-B HLA class I histocompatibility | 6293 |
| | antigen, B-8 alpha | |
| Q9BVS4_C449 | RIOK2 Serine/threonine-protein kinase | 6294 |
| | RIO2 | |
| O15027_C1273 | SEC16A Protein transport protein | 6295 |
| | Sec16A | |
| O15027_C1619 | SEC16A Protein transport protein | 6296 |
| | Sec16A | |
| Q9NQW7_C279 | XPNPEP1 Xaa-Pro aminopeptidase 1 | 6297 |
| Q96EB6_C380 | SIRT1 NAD-dependent protein | 6298 |
| | deacetylase sirtuin-1 | |
| P10398_C514 | ARAF Serine/threonine-protein kinase | 6299 |
| | A-Raf | |
| Q15046_C456 | KARS Lysine--tRNA ligase | 6300 |
| Q15046_C534 | KARS Lysine--tRNA ligase | 6301 |
| Q12802_C1548 | AKAP13 A-kinase anchor protein 13 | 6302 |
| Q12802_C2101 | AKAP13 A-kinase anchor protein 13 | 6303 |
| Q9H7B4_C421 | SMYD3 SET and MYND domain- | 6304 |
| | containing protein 3 | |
| Q13867_C40 | BLMH Bleomycin hydrolase | 6305 |
| Q13867_C189 | BLMH Bleomycin hydrolase | 6306 |
| Q13404_C71 | UBE2V1 Ubiquitin-conjugating | 6307 |
| | enzyme E2 variant 1 | |
| Q9BVA0_C344 | KATNB1 Katanin p80 WD40- | 6308 |
| | containing subunit B1 | |
| O60504_C521 | SORBS3 Vinexin | 6309 |
| P07437_C127 | TUBB Tubulin beta chain | 6310 |
| Q96SW2_C287 | CRBN Protein cereblon | 6311 |
| Q9Y490_C1023 | TLN1 Talin-1 | 6312 |
| Q9Y490_C2161 | TLN1 Talin-1 | 6313 |
| Q86W56_C963 | PARG Poly(ADP-ribose) | 6314 |
| | glycohydrolase | |
| P49005_C83 | POLD2 DNA polymerase delta subunit | 6315 |
| | 2 | |
| P49005_C404 | POLD2 DNA polymerase delta subunit | 6316 |
| | 2 | |
| P49006_C134 | MARCKSL1 MARCKS-related protein | 6317 |
| Q05932_C209 | FPGS Folylpolyglutamate synthase, | 6318 |
| | mitochondrial | |
| Q9Y3D6_C41 | FIS1 Mitochondrial fission 1 protein | 6319 |
| O94916_C270 | NFAT5 Nuclear factor of activated T- | 6320 |
| | cells 5 | |
| P27708_C1374 | CAD CAD protein | 6321 |
| Q96LB3_C53 | IFT74 Intraflagellar transport protein 74 | 6322 |
| | homolog | |
| Q96EY9_C13 | ADAT3 tRNA-specific adenosine | 6323 |
| | deaminase-like protein 3 | |
| O75608_C211 | LYPLA1 Acyl-protein thioesterase 1 | 6324 |
| P22033_C433 | MUT Methylmalonyl-CoA mutase, | 6325 |
| | mitochondrial | |
| O43290_C560 | SART1 U4/U6.U5 tri-snRNP- | 6326 |
| | associated protein 1 | |
| O43617_C31 | TRAPPC3 Trafficking protein particle | 6327 |
| | complex subunit 3 | |
| Q9P258_C337 | RCC2 Protein RCC2 | 6328 |
| P61313_C110 | RPL15 60S ribosomal protein L15 | 6329 |
| O95684_C244 | FGFR1OP FGFR1 oncogene partner | 6330 |
| Q17RN3_C36 | FAM98C Protein FAM98C | 6331 |
| P11498_C372 | PC Pyruvate carboxylase, | 6332 |
| | mitochondrial | |
| Q13263_C152 | TRIM28 Transcription intermediary | 6333 |
| | factor 1-beta | |
| Q13263_C717 | TRIM28 Transcription intermediary | 6334 |
| | factor 1-beta | |
| O75131_C385 | CPNE3 Copine-3 | 6335 |
| Q16762_C64 | TST Thiosulfate sulfurtransferase | 6336 |
| Q02790_C103 | FKBP4 Peptidyl-prolyl cis-trans | 6337 |
| | isomerase FKBP4 | |
| Q02790_C342 | FKBP4 Peptidyl-prolyl cis-trans | 6338 |
| | isomerase FKBP4 | |
| P26358_C41 | DNMT1 DNA (cytosine-5)- | 6339 |
| | methyltransferase 1 | |
| P26358_C580 | DNMT1 DNA (cytosine-5)- | 6340 |
| | methyltransferase 1 | |
| Q8TEM1_C767 | NUP210 Nuclear pore membrane | 6341 |
| | glycoprotein 210 | |
| P61006_C23 | RAB8A Ras-related protein Rab-8A | 6342 |
| O15446_C55 | CD3EAP DNA-directed RNA | 6343 |
| | polymerase I subunit RPA34 | |
| P54136_C115 | RARS Arginine--tRNA ligase, | 6344 |
| | cytoplasmic | |
| P36551_C319 | CPOX Coproporphyrinogen-III | 6345 |
| | oxidase, mitochondrial | |
| Q86YV5_C155 | SGK223 Tyrosine-protein kinase | 6346 |
| | SgK223 | |
| Q86YV5_C221 | SGK223 Tyrosine-protein kinase | 6347 |
| | SgK223 | |
| Q96PV6_C529 | LENG8 Leukocyte receptor cluster | 6348 |
| | member 8 | |
| P30520_C182 | ADSS Adenylosuccinate synthetase | 6349 |
| | isozyme 2 | |
| Q9NV88_C578 | INTS9 Integrator complex subunit 9 | 6350 |
| P49589_C182 | CARS Cysteine--tRNA ligase, | 6351 |
| | cytoplasmic | |
| P15374_C50 | UCHL3 Ubiquitin carboxyl-terminal | 6352 |
| | hydrolase isozyme L3 | |
| P55735_C299 | SEC13 Protein SEC13 homolog | 6353 |
| Q9NR09_C1547 | BIRC6 Baculoviral IAP repeat- | 6354 |
| | containing protein 6 | |
| A8MVX7_C100 | Uncharacterized protein | 6355 |
| P56192_C333 | MARS Methionine--tRNA ligase, | 6356 |
| | cytoplasmic | |
| Q8NAG6_C517 | ANKLE1 Ankyrin repeat and LEM | 6357 |
| | domain-containing protein 1 | |
| P55884_C302 | EIF3B Eukaryotic translation initiation | 6358 |
| | factor 3 subunit | |
| Q86V15_C35 | CASZ1 Zinc finger protein castor | 6359 |
| | homolog 1 | |
| Q9H2G2_C1153 | SLK STE20-like serine/threonine- | 6360 |
| | protein kinase | |
| Q99570_C899 | PIK3R4 Phosphoinositide 3-kinase | 6361 |
| | regulatory subunit 4 | |
| Q9BVP2_C251 | GNL3 Guanine nucleotide-binding | 6362 |
| | protein-like 3 | |
| Q13813_C1454 | SPTAN1 Spectrin alpha chain, non- | 6363 |
| | erythrocytic 1 | |
| Q6P3S1_C21 | DENND1B DENN domain-containing | 6364 |
| | protein 1B | |
| P07737_C71 | PFN1 Profilin-1 | 6365 |
| O00425_C546 | IGF2BP3 Insulin-like growth factor 2 | 6366 |
| | mRNA-binding protein | |
| Q9H832_C154 | UBE2Z Ubiquitin-conjugating enzyme | 6367 |
| | E2 Z | |
| Q9H832_C261 | UBE2Z Ubiquitin-conjugating enzyme | 6368 |
| | E2 Z | |
| Q9UKV8_C188 | EIF2C2 Protein argonaute-2 | 6369 |
| Q00796_C350 | SORD Sorbitol dehydrogenase | 6370 |
| Q9Y376_C121 | CAB39 Calcium-binding protein 39 | 6371 |
| P00492_C66 | HPRT1 Hypoxanthine-guanine | 6372 |
| | phosphoribosyltransferase | |
| Q9NVR5_C727 | DNAAF2 Protein kintoun | 6373 |
| P15498_C652 | VAV1 Proto-oncogene vav | 6374 |
| Q92499_C668 | DDX1 ATP-dependent RNA helicase | 6375 |
| | DDX1 | |
| P08559_C261 | PDHA1 Pyruvate dehydrogenase E1 | 6376 |
| | component subunit alpha, | |
| Q8IUY3_C274 | GRAMD2 GRAM domain-containing | 6377 |
| | protein 2 | |
| Q96PE3_C854 | INPP4A Type I inositol 3,4- | 6378 |
| | bisphosphate 4-phosphatase | |
| Q9NPD8_C86 | UBE2T Ubiquitin-conjugating enzyme | 6379 |
| | E2 T | |
| Q9H307_C439 | PNN Pinin | 6380 |
| P46939_C2003 | UTRN Utrophin | 6381 |
| P46939_C2098 | UTRN Utrophin | 6382 |
| P46939_C3076 | UTRN Utrophin | 6383 |
| Q04637_C934 | EIF4G1 Eukaryotic translation | 6384 |
| | initiation factor 4 gamma 1 | |
| D6RAD4_C204 | CDK7 Cyclin-dependent kinase 7 | 6385 |
| P52701_C579 | MSH6 DNA mismatch repair protein | 6386 |
| | Msh6 | |
| O60292_C1322 | SIPA1L3 Signal-induced proliferation- | 6387 |
| | associated 1-like protein | |
| Q16831_C17 | UPP1 Uridine phosphorylase 1 | 6388 |
| P49368_C372 | CCT3 T-complex protein 1 subunit | 6389 |
| | gamma | |
| O75962_C1717 | TRIO Triple functional domain protein | 6390 |
| O75962_C2327 | TRIO Triple functional domain protein | 6391 |
| Q53H82_C100 | LACTB2 Beta-lactamase-like protein 2 | 6392 |
| Q5TC12_C321 | ATPAF1 ATP synthase mitochondrial | 6393 |
| | F1 complex assembly factor 1 | |
| P33992_C221 | MCM5 DNA replication licensing | 6394 |
| | factor MCM5 | |
| P12081_C455 | HARS Histidine--tRNA ligase, | 6395 |
| | cytoplasmic | |
| Q15418_C432 | RPS6KA1 Ribosomal protein S6 kinase | 6396 |
| | alpha-1 | |
| P49590_C236 | HARS2 Probable histidine--tRNA | 6397 |
| | ligase, mitochondrial | |
| O14893_C63 | GEMIN2 Gem-associated protein 2 | 6398 |
| Q9H0D6_C557 | XRN2 5-3 exoribonuclease 2 | 6399 |
| Q4G0F5_C334 | VPS26B Vacuolar protein sorting- | 6400 |
| | associated protein 26B | |
| P08567_C59 | PLEK Pleckstrin | 6401 |
| P50135_C196 | HNMT Histamine N-methyltransferase | 6402 |
| Q9BXF6_C66 | RAB11FIP5 Rab11 family-interacting | 6403 |
| | protein 5 | |
| P51668_C111 | UBE2D1 Ubiquitin-conjugating | 6404 |
| | enzyme E2 D1 | |
| Q5T0L3_C212 | C1orf111 Uncharacterized protein | 6405 |
| | C1orf111 | |
| A6NHR9_C897 | SMCHD1 Structural maintenance of | 6406 |
| | chromosomes flexible hinge domain | |
| | containing 1 | |
| A6NHR9_C1899 | SMCHD1 Structural maintenance of | 6407 |
| | chromosomes flexible hinge domain | |
| | containing 1 | |
| Q12824_C167 | SMARCB1 SWI/SNF-related matrix- | 6408 |
| | associated actin-dependent | |
| Q969R8_C438 | ITFG2 Integrin-alpha FG-GAP repeat- | 6409 |
| | containing protein 2 | |
| P17544_C61 | ATF7 Cyclic AMP-dependent | 6410 |
| | transcription factor ATF-7 | |
| O00410_C1057 | IPO5 Importin-5 | 6411 |
| Q9BT25_C354 | HAUS8 HAUS augmin-like complex | 6412 |
| | subunit 8 | |
| O95861_C206 | BPNT1 3(2),5-bisphosphate | 6413 |
| | nucleotidase 1 | |
| O15260_C32 | SURF4 Surfeit locus protein 4 | 6414 |
| P35580_C1238 | MYH10 Myosin-10 | 6415 |
| P35270_C171 | SPR Sepiapterin reductase | 6416 |
| P42330_C193 | AKR1C3 Aldo-keto reductase family 1 | 6417 |
| | member C3 | |
| P42331_C302 | ARHGAP25 Rho GTPase-activating | 6418 |
| | protein 25 | |
| Q9Y230_C227 | RUVBL2 RuvB-like 2 | 6419 |
| Q92621_C877 | NUP205 Nuclear pore complex protein | 6420 |
| | Nup205 | |
| Q92621_C1297 | NUP205 Nuclear pore complex protein | 6421 |
| | Nup205 | |
| P42025_C222 | ACTR1B Beta-centractin | 6422 |
| Q5H9R7_C844 | PPP6R3 Serine/threonine-protein | 6423 |
| | phosphatase 6 regulatory | |
| Q02241_C447 | KIF23 Kinesin-like protein KIF23 | 6424 |
| O94915_C888 | FRYL Protein furry homolog-like | 6425 |
| P53621_C245 | COPA Coatomer subunit alpha | 6426 |
| P53621_C522 | COPA Coatomer subunit alpha | 6427 |
| O43678_C58 | NDUFA2 NADH dehydrogenase | 6428 |
| Q14116_C74 | IL18 Interleukin-18 | 6429 |
| Q86Y37_C362 | CACUL1 CDK2-associated and cullin | 6430 |
| | domain-containing protein | |
| Q99704_C115 | DOK1 Docking protein 1 | 6431 |
| O75153_C214 | KIAA0664 Clustered mitochondria | 6432 |
| | protein homolog | |
| Q99961_C147 | SH3GL1 Endophilin-A2 | 6433 |
| O43294_C416 | TGFB1I1 Transforming growth factor | 6434 |
| | beta-1-induced transcript 1 | |
| Q7Z7H8_C213 | MRPL10 39S ribosomal protein L10, | 6435 |
| | mitochondrial | |
| Q96IF1_C182 | AJUBA LIM domain-containing | 6436 |
| | protein ajuba | |
| Q8NC26_C51 | ZNF114 Zinc finger protein 114 | 6437 |
| Q8NC26_C136 | ZNF114 Zinc finger protein 114 | 6438 |
| P21266_C39 | GSTM3 Glutathione S-transferase Mu 3 | 6439 |
| Q8WXA9_C494 | SREK1 Splicing regulatory | 6440 |
| | glutamine/lysine-rich protein | |

Table 4 illustrates an exemplary list of cysteine-containing proteins identified in a human T cell. Table 4 further shows the accession number (or the protein identifier) of the protein, cysteine residue number, and an illustrative peptide fragment containing the cysteine of interest (denoted by C*).

TABLE 4

| | | SEQ |
|---|---|---|
| | | ID |
| Identifier | Protein Name | NO: |
| Q13418_C239 | ILK Integrin-linked protein kinase | 6441 |
| P07203_C156 | GPX1 Glutathione peroxidase 1 | 6442 |
| P00390_C102 | GSR Glutathione reductase, | 6443 |
| | mitochondrial | |
| P07203_C78 | GPX1 Glutathione peroxidase 1 | 6444 |
| Q9BRA2_C43 | TXNDC17 Thioredoxin domain- | 6445 |
| | containing protein 17 | |
| Q01844_C524 | EWSR1 RNA-binding protein EWS | 6446 |
| Q99798_C385 | ACO2 Aconitate hydratase, | 6447 |
| | mitochondrial | |
| P18031_C215 | PTPN1 Tyrosine-protein phosphatase | 6448 |
| | non-receptor type 1 | |
| Q9Y2R4_C536 | DDX52 Probable ATP-dependent RNA | 6449 |
| | helicase DDX52 | |
| P53621_C921 | COPA Coatomer subunit alpha | 6450 |
| P31930_C380 | UQCRC1 Cytochrome b-c1 complex | 6451 |
| | subunit 1, mitochondrial | |
| P09211_C48 | GSTP1 Glutathione S-transferase P | 6452 |
| P46459_C264 | NSF Vesicle-fusing ATPase | 6453 |
| Q9NR56_C43 | MBNL1 Muscleblind-like protein 1 | 6454 |
| Q5VZF2_C43 | MBNL2 Muscleblind-like protein 2 | 6455 |
| P49368_C455 | CCT3 T-complex protein 1 subunit | 6456 |
| | gamma | |
| P47756_C62 | CAPZB F-actin-capping protein | 6457 |
| | subunit beta | |
| P57737_C42 | CORO7 Coronin-7 | 6458 |
| Q9BV79_C263 | MECR Trans-2-enoyl-CoA reductase, | 6459 |
| | mitochondrial | |
| P13010_C339 | XRCC5 X-ray repair cross- | 6460 |
| | complementing protein 5 | |
| Q9H2U2_C302 | PPA2 Inorganic pyrophosphatase 2, | 6461 |
| | mitochondrial | |
| P13010_C346 | XRCC5 X-ray repair cross- | 6462 |
| | complementing protein 5 | |
| Q16875_C102 | PFKFB3 6-phosphofructo-2- | 6463 |
| | kinase/fructose-2,6-bisphosphata | |
| P14866_C452 | HNRNPL Heterogeneous nuclear | 6464 |
| | ribonucleoprotein L | |
| Q99798_C448 | ACO2 Aconitate hydratase, | 6465 |
| | mitochondrial | |
| Q99798_C451 | ACO2 Aconitate hydratase, | 6466 |
| | mitochondrial | |
| Q9C0B1_C326 | FTO Alpha-ketoglutarate-dependent | 6467 |
| | dioxygenase FTO | |
| Q92947_C115 | GCDH Glutaryl-CoA dehydrogenase, | 6468 |
| | mitochondrial | |
| Q92597_C168 | NDRG1 Protein NDRG1 | 6469 |
| P09874_C298 | PARP1 Polymerase 2 | 6470 |
| P55036_C58 | PSMD4 26S proteasome non-ATPase | 6471 |
| | regulatory subunit 4 | |
| A6NHR9_C1433 | SMCHD1 Structural maintenance of | 6472 |
| | chromosomes flexible hinge domain- | |
| | containing protein 1 | |
| Q2M2I8_C270 | AAK1 AP2-associated protein kinase 1 | 6473 |
| O14880_C56 | MGST3 Microsomal glutathione S- | 6474 |
| | transferase 3 | |
| Q9C0J8_C120 | WDR33 pre-mRNA 3 end processing | 6475 |
| | protein WDR33 | |
| Q96ME7_C430 | ZNF512 Zinc finger protein 512 | 6476 |
| Q01813_C641 | PFKP 6-phosphofructokinase type C | 6477 |
| P21333_C810 | FLNA Filamin-A | 6478 |
| P07858_C108 | CTSB Cathepsin B | 6479 |
| Q9UGI8_C22 | TES Testin | 6480 |
| P48047_C141 | ATP5O ATP synthase subunit O, | 6481 |
| | mitochondrial | |
| P27707_C59 | DCK Deoxycytidine kinase | 6482 |
| Q9C0J8_C249 | WDR33 pre-mRNA 3 end processing | 6483 |
| | protein WDR33 | |
| Q9UMS4_C298 | PRPF19 Pre-mRNA-processing factor | 6484 |
| | 19 | |
| P25705_C294 | ATP5A1 ATP synthase subunit alpha, | 6485 |
| | mitochondrial | |
| H3BQZ7_C518 | Uncharacterized protein | 6486 |
| Q1KMD3_C518 | HNRNPUL2 Heterogeneous nuclear | 6487 |
| | ribonucleoprotein U-like protein 2 | |
| Q8IW45_C82 | CARKD ATP-dependent (S)- | 6488 |
| | NAD(P)H-hydrate dehydratase | |
| P09110_C177 | ACAA1 3-ketoacyl-CoA thiolase, | 6489 |
| | peroxisomal | |
| P34949_C11 | MPI Mannose-6-phosphate isomerase | 6490 |
| Q7RTV0_C49 | PHF5A PHD finger-like domain- | 6491 |
| | containing protein 5A | |
| Q9BWD1_C65 | ACAT2 Acetyl-CoA acetyltransferase, | 6492 |
| | cytosolic | |
| P48735_C154 | IDH2 Isocitrate dehydrogenase | 6493 |
| P51665_C116 | PSMD7 26S proteasome non-ATPase | 6494 |
| | regulatory subunit 7 | |
| Q6YN16_C218 | HSDL2 Hydroxysteroid | 6495 |
| | dehydrogenase-like protein 2 | |
| P14866_C581 | HNRNPL Heterogeneous nuclear | 6496 |
| | ribonucleoprotein L | |
| P78417_C32 | GSTO1 Glutathione S-transferase | 6497 |
| | omega-1 | |
| P51610_C1872 | HCFC1 Host cell factor 1 | 6498 |
| Q86WV6_C206 | TMEM173 Transmembrane protein | 6499 |
| | 173 | |
| P08559_C181 | PDHA1 Pyruvate dehydrogenase E1 | 6500 |
| | component subunit alpha, somatic | |
| | form, mitochondrial | |
| O60318_C1285 | MCM3AP 80 kDa MCM3-associated | 6501 |
| | protein | |
| P78527_C4106 | PRKDC DNA-dependent protein | 6502 |
| | kinase catalytic subunit | |
| P31943_C34 | HNRNPH1 Heterogeneous nuclear | 6503 |
| | ribonucleoprotein H | |
| P62333_C170 | PSMC6 26S protease regulatory | 6504 |
| | subunit 10B | |
| O14776_C1062 | TCERG1 Transcription elongation | 6505 |
| | regulator 1 | |
| Q9Y3Z3_C341 | SAMHD1 SAM domain and HD | 6506 |
| | domain-containing protein 1 | |
| Q96GM5_C460 | SMARCD1 SWI/SNF-related matrix- | 6507 |
| | associated actin-dependent | |
| P29350_C361 | PTPN6 Tyrosine-protein phosphatase | 6508 |
| | non-receptor type 6 | |
| P14625_C645 | HSP90B1 Endoplasmin | 6509 |
| P10515_C586 | DLAT Dihydrolipoyllysine-residue | 6510 |
| | acetyltransferase component of | |
| | pyruvate dehydrogenase complex, | |
| | mitochondrial | |
| P02042_C94 | HBD Hemoglobin subunit delta | 6511 |
| P14174_C81 | MIF Macrophage migration inhibitory | 6512 |
| | factor | |
| P04083_C343 | ANXA1 Annexin A1 | 6513 |
| P04406_C247 | GAPDH Glyceraldehyde-3-phosphate | 6514 |
| | dehydrogenase | |
| O94901_C657 | SUN1 SUN domain-containing protein | 6515 |
| | 1 | |
| Q9UH99_C563 | SUN2 SUN domain-containing protein | 6516 |
| | 2 | |
| P13796_C42 | LCP1 Plastin-2 | 6517 |
| O95985_C217 | TOP3B DNA topoisomerase 3-beta-1 | 6518 |
| P00367_C376 | GLUD1 Glutamate dehydrogenase 1, | 6519 |
| | mitochondrial | |
| P49840_C380 | GSK3A Glycogen synthase kinase-3 | 6520 |
| | alpha | |
| P12814_C154 | ACTN1 Alpha-actinin-1 | 6521 |
| P55084_C458 | HADHB Trifunctional enzyme subunit | 6522 |
| | beta, mitochondrial | |
| P63220_C56 | RPS21 40S ribosomal protein S21 | 6523 |
| O43707_C173 | ACTN4 Alpha-actinin-4 | 6524 |
| Q49A26_C356 | GLYR1 Putative oxidoreductase | 6525 |
| | GLYR1 | |
| Q9NYL9_C231 | TMOD3 Tropomodulin-3 | 6526 |
| Q63HN8_C2943 | RNF213 E3 ubiquitin-protein ligase | 6527 |
| | RNF213 | |
| P27987_C740 | ITPKB Inositol-trisphosphate 3-kinase | 6528 |
| | B | |
| Q96F15_C185 | GIMAP5 GTPase IMAP family | 6529 |
| | member 5 | |
| Q13501_C131 | SQSTM1 Sequestosome-1 | 6530 |
| P00441_C147 | SOD1 Superoxide dismutase | 6531 |
| Q92616_C1362 | GCN1L1 Translational activator GCN1 | 6532 |
| Q08J23_C321 | NSUN2 tRNA (cytosine(34)-C(5))- | 6533 |
| | methyltransferase | |
| Q01518_C416 | CAP1 Adenylyl cyclase-associated | 6534 |
| | protein 1 | |
| P55735_C245 | SEC13 Protein SEC13 homolog | 6535 |
| P08133_C59 | ANXA6 Annexin A6 | 6536 |
| P62910_C96 | RPL32 60S ribosomal protein L32 | 6537 |
| P62306_C66 | SNRPF Small nuclear | 6538 |
| | ribonucleoprotein F | |
| I3L420_C311 | LSM14A Protein LSM14 homolog A | 6539 |
| Q8ND56_C375 | LSM14A Protein LSM14 homolog A | 6540 |
| P57737_C187 | CORO7 Coronin-7 | 6541 |
| Q96FS4_C609 | SIPA1 Signal-induced proliferation- | 6542 |
| | associated protein 1 | |
| Q8TD19_C487 | NEK9 Serine/threonine-protein kinase | 6543 |
| | Nek9 | |
| Q99567_C561 | NUP88 Nuclear pore complex protein | 6544 |
| | Nup88 | |
| P21333_C631 | FLNA Filamin-A | 6545 |
| P26583_C23 | HMGB2 High mobility group protein | 6546 |
| | B2 | |
| P09429_C23 | HMGB1 High mobility group protein | 6547 |
| | B1 | |
| Q99460_C898 | PSMD1 26S proteasome non-ATPase | 6548 |
| | regulatory subunit 1 | |
| Q92785_C53 | DPF2 Zinc finger protein ubi-d4 | 6549 |
| Q16822_C431 | PCK2 Phosphoenolpyruvate | 6550 |
| | carboxykinase | |
| O95671_C441 | ASMTL N-acetylserotonin O- | 6551 |
| | methyltransferase-like protein | |
| Q9UGI8_C412 | TES Testin | 6552 |
| Q13574_C905 | DGKZ Diacylglycerol kinase zeta | 6553 |
| Q9UGI8_C416 | TES Testin | 6554 |
| Q9NZA1_C191 | CLIC5 Chloride intracellular channel | 6555 |
| | protein 5 | |
| P43354_C566 | NR4A2 Nuclear receptor subfamily 4 | 6556 |
| | group A member 2 | |
| P22736_C566 | NR4A1 Nuclear receptor subfamily 4 | 6557 |
| | group A member 1 | |
| Q96EP0_C504 | RNF31 E3 ubiquitin-protein ligase | 6558 |
| | RNF31 | |
| P30048_C229 | PRDX3 Thioredoxin-dependent | 6559 |
| | peroxide reductase, mitochondrial | |
| P21333_C210 | FLNA Filamin-A | 6560 |
| Q08AF3_C627 | SLFN5 Schlafen family member 5 | 6561 |
| Q92608_C465 | DOCK2 Dedicator of cytokinesis | 6562 |
| | protein 2 | |
| O75592_C1868 | MYCBP2 Probable E3 ubiquitin- | 6563 |
| | protein ligase MYCBP2 | |
| P21333_C205 | FLNA Filamin-A | 6564 |
| P21333_C2160 | FLNA Filamin-A | 6565 |
| P30626_C57 | SRI Sorcin | 6566 |
| Q9P2T1_C348 | GMPR2 GMP reductase 2 | 6567 |
| Q52LJ0_C295 | FAM98B Protein FAM98B | 6568 |
| Q13813_C956 | SPTAN1 Spectrin alpha chain, non- | 6569 |
| | erythrocytic 1 | |
| Q8NCA5_C293 | FAM98A Protein FAM98A | 6570 |
| Q96G03_C573 | PGM2 Phosphoglucomutase-2 | 6571 |
| O75832_C107 | PSMD10 26S proteasome non-ATPase | 6572 |
| | regulatory subunit 10 | |
| O95372_C213 | LYPLA2 Acyl-protein thioesterase 2 | 6573 |
| Q5JVS0_C22 | HABP4 Intracellular hyaluronan- | 6574 |
| | binding protein 4 | |
| Q9BPZ7_C149 | MAPKAP1 Target of rapamycin | 6575 |
| | complex 2 subunit MAPKAP1 | |
| Q8NE71_C655 | ABCF1 ATP-binding cassette sub- | 6576 |
| | family F member 1 | |
| P48059_C100 | LIMS1 LIM and senescent cell antigen- | 6577 |
| | like-containing domain 1 | |
| Q13263_C83 | TRIM28 Transcription intermediary | 6578 |
| | factor 1-beta | |
| Q13263_C88 | TRIM28 Transcription intermediary | 6579 |
| | factor 1-beta | |
| Q13263_C91 | TRIM28 Transcription intermediary | 6580 |
| | factor 1-beta | |
| Q9Y4H4_C116 | GPSM3 G-protein-signaling modulator | 6581 |
| | 3 | |
| O00139_C537 | KIF2A Kinesin-like protein KIF2A | 6582 |
| A6NHR9_C897 | SMCHD1 Structural maintenance of | 6583 |
| | chromosomes flexible hinge domain | |
| | containing 1 | |
| Q12824_C350 | SMARCB1 SWI/SNF-related matrix- | 6584 |
| | associated actin-dependent | |
| Q96EP5_C85 | DAZAP1 DAZ-associated protein 1 | 6585 |
| Q96ME7_C413 | ZNF512 Zinc finger protein 512 | 6586 |
| Q13813_C1622 | SPTAN1 Spectrin alpha chain, non- | 6587 |
| | erythrocytic 1 | |
| Q8WWQ0_C28 | PHIP PH-interacting protein | 6588 |
| P22736_C303 | NR4A1 Nuclear receptor subfamily 4 | 6589 |
| | group A member 1 | |
| Q9P2N6_C459 | KANSL3 KAT8 regulatory NSL | 6590 |
| | complex subunit 3 | |
| Q93084_C447 | ATP2A3 Sarcoplasmic/endoplasmic | 6591 |
| | reticulum calcium ATPase | |
| Q92945_C436 | KHSRP Far upstream element-binding | 6592 |
| | protein 2 | |
| O75643_C1278 | SNRNP200 U5 small nuclear | 6593 |
| | ribonucleoprotein 200 kDa helicase | |
| P14866_C260 | HNRNPL Heterogeneous nuclear | 6594 |
| | ribonucleoprotein L | |
| H7BZ11_C83 | Uncharacterized protein | 6595 |
| H7BZ11_C88 | Uncharacterized protein | 6596 |
| Q969Q0_C72 | RPL36AL 60S ribosomal protein L36a- | 6597 |
| | like | |
| Q969Q0_C77 | RPL36AL 60S ribosomal protein L36a- | 6598 |
| | like | |
| P14866_C261 | HNRNPL Heterogeneous nuclear | 6599 |
| | ribonucleoprotein L | |
| P15927_C219 | RPA2 Replication protein A 32 kDa | 6600 |
| | subunit | |
| Q92598_C290 | HSPH1 Heat shock protein 105 kDa | 6601 |
| P22307_C71 | SCP2 Non-specific lipid-transfer | 6602 |
| | protein | |
| P26358_C896 | DNMT1 DNA (cytosine-5)- | 6603 |
| | methyltransferase 1 | |
| Q06830_C173 | PRDX1 Pcroxiredoxin-1 | 6604 |
| P42765_C287 | ACAA2 3-ketoacyl-CoA thiolase, | 6605 |
| | mitochondrial | |
| O75592_C2163 | MYCBP2 Probable E3 ubiquitin- | 6606 |
| | protein ligase MYCBP2 | |
| Q9Y4W2_C504 | LAS1L Ribosomal biogenesis protein | 6607 |
| | LAS1L | |
| A6NHR9_C1856 | SMCHD1 Structural maintenance of | 6608 |
| | chromosomes flexible hinge domain | |
| | containing 1 | |
| O75351_C240 | VPS4B Vacuolar protein sorting- | 6609 |
| | associated protein 4B | |
| P52333_C811 | JAK3 Tyrosine-protein kinase JAK3 | 6610 |
| P52564_C216 | MAP2K6 Dual specificity mitogen- | 6611 |
| | activated protein kinase | |
| P31146_C40 | CORO1A Coronin-1A | 6612 |
| P55072_C522 | VCP Transitional endoplasmic | 6613 |
| | reticulum ATPase | |
| P21333_C2293 | FLNA Filamin-A | 6614 |
| Q15233_C208 | NONO Non-POU domain-containing | 6615 |
| | octamer-binding protein | |
| P00492_C23 | HPRT1 Hypoxanthine-guanine | 6616 |
| | phosphoribosyltransferase | |
| O94804_C873 | STK10 Serine/threonine-protein kinase | 6617 |
| | 10 | |
| P36578_C96 | RPL4 60S ribosomal protein L4 | 6618 |
| P28838_C462 | LAP3 Cytosol aminopeptidase | 6619 |
| P52272_C676 | HNRNPM Heterogeneous nuclear | 6620 |
| | ribonucleoprotein M | |
| Q08AF3_C489 | SLFN5 Schlafen family member 5 | 6621 |
| Q9Y383_C59 | LUC7L2 Putative RNA-binding protein | 6622 |
| | Luc7-like 2 | |
| Q9NZ09_C45 | UBAP1 Ubiquitin-associated protein 1 | 6623 |
| Q14152_C404 | EIF3A Eukaryotic translation initiation | 6624 |
| | factor 3 subunit | |
| Q99496_C72 | RNF2 E3 ubiquitin-protein ligase | 6625 |
| | RING2 | |
| P30086_C133 | PEBP1 Phosphatidylethanolamine- | 6626 |
| | binding protein 1 | |
| Q93009_C90 | USP7 Ubiquitin carboxyl-terminal | 6627 |
| | hydrolase 7 | |
| P83731_C36 | RPL24 60S ribosomal protein L24 | 6628 |
| P83731_C6 | RPL24 60S ribosomal protein L24 | 6629 |
| Q92499_C110 | DDX1 ATP-dependent RNA helicase | 6630 |
| | DDX1 | |
| A6NHR9_C492 | SMCHD1 Structural maintenance of | 6631 |
| | chromosomes flexible hinge domain | |
| | containing 1 | |
| P34932_C290 | HSPA4 Heat shock 70 kDa protein 4 | 6632 |
| Q9H4B7_C12 | TUBB1 Tubulin beta-1 chain | 6633 |
| Q6PJG6_C326 | BRAT1 BRCA1-associated ATM | 6634 |
| | activator 1 | |
| Q9NZ08_C486 | ERAP1 Endoplasmic reticulum | 6635 |
| | aminopeptidase 1 | |
| Q5VZF2_C19 | MBNL2 Muscleblind-like protein 2 | 6636 |
| Q9NR56_C19 | MBNL1 Muscleblind-like protein 1 | 6637 |
| P55263_C353 | ADK Adenosine kinase | 6638 |
| Q8WUM4_C40 | PDCD61P Programmed cell death 6- | 6639 |
| | interacting protein | |
| P50579_C121 | METAP2 Methionine aminopeptidase 2 | 6640 |
| P21333_C1165 | FLNA Filamin-A | 6641 |
| Q2M2I8_C288 | AAK1 AP2-associated protein kinase 1 | 6642 |
| Q8N0W3_C484 | FUK L-fucose kinase | 6643 |
| P18621_C144 | RPL17 60S ribosomal protein L17 | 6644 |
| J3QL51_C144 | RPL17-C18ORF32 Protein RPL17- | 6645 |
| | C18ORF32 | |
| Q12906_C203 | ILF3 Interleukin enhancer-binding | 6646 |
| | factor 3 | |
| Q8N163_C754 | KIAA1967 DBIRD complex subunit | 6647 |
| | KIAA1967 | |
| Q6P1R4_C213 | DUS1L tRNA-dihydrouridine(16/17) | 6648 |
| | synthase | |
| Q9Y490_C2196 | TLN1 Talin-1 | 6649 |
| Q92947_C176 | GCDH Glutaryl-CoA dehydrogenase, | 6650 |
| | mitochondrial | |
| P21333_C1453 | FLNA Filamin-A | 6651 |
| P49411_C147 | TUFM Elongation factor Tu, | 6652 |
| | mitochondrial | |
| Q9BV38_C384 | WDR18 WD repeat-containing protein | 6653 |
| | 18 | |
| Q93008_C1566 | USP9X Probable ubiquitin carboxyl- | 6654 |
| | terminal hydrolase FAF | |
| O00507_C1568 | USP9Y Probable ubiquitin carboxyl- | 6655 |
| | terminal hydrolase FAF | |
| Q9BXL7_C1115 | CARD11 Caspase recruitment domain- | 6656 |
| | containing protein 11 | |
| Q9P2J5_C573 | LARS Leucine--tRNA ligase, | 6657 |
| | cytoplasmic | |
| O00541_C391 | PES1 Pescadillo homolog | 6658 |
| Q9Y228_C388 | TRAF3IP3 TRAF3-interacting JNK- | 6659 |
| | activating modulator | |
| O95747_C191 | OXSR1 Serine/threonine-protein kinase | 6660 |
| | OSR1 | |
| Q9UEW8_C237 | STK39 STE20/SPS1-related proline- | 6661 |
| | alanine-rich protein kinase | |
| Q8IVH4_C184 | MMAA Methylmalonic aciduria type A | 6662 |
| | protein, mitochondrial | |
| P49189_C484 | ALDH9A1 4- | 6663 |
| | trimethylaminobutyraldehyde | |
| | dehydrogenase | |
| P08238_C521 | HSP90AB1 Heat shock protein HSP | 6664 |
| | 90-beta | |
| Q8TDP1_C34 | RNASEH2C Ribonuclease H2 subunit | 6665 |
| | C | |
| P08575_C760 | PTPRC Receptor-type tyrosine-protein | 6666 |
| | phosphatase C | |
| I3L3Q1_C558 | Uncharacterized protein | 6667 |
| Q6ZSZ5_C600 | ARHGEF18 Rho guanine nucleotide | 6668 |
| | exchange factor 18 | |
| Q9Y490_C1661 | TLN1 Talin-1 | 6669 |
| Q9Y490_C1671 | TLN1 Talin-1 | 6670 |
| P53384_C235 | NUBP1 Cytosolic Fe—S cluster | 6671 |
| | assembly factor NUBP1 | |
| P49589_C204 | CARS Cysteine--tRNA ligase, | 6672 |
| | cytoplasmic | |
| P78527_C2469 | PRKDC DNA-dependent protein | 6673 |
| | kinase catalytic subunit | |
| Q9P2R3_C460 | ANKFY1 Ankyrin repeat and FYVE | 6674 |
| | domain-containing protein | |
| P33316_C222 | DUT Deoxyuridine 5-triphosphate | 6675 |
| | nucleotidohydrolase, mitochondrial | |
| E9PMF8_C694 | PTPRC Receptor-type tyrosine-protein | 6676 |
| | phosphatase C | |
| P49773_C84 | HINT1 Histidine triad nucleotide- | 6677 |
| | binding protein 1 | |
| Q9BRZ2_C514 | TRIM56 E3 ubiquitin-protein ligase | 6678 |
| | TRIM56 | |
| P35754_C83 | GLRX Glutaredoxin-1 | 6679 |
| Q9Y490_C1353 | TLN1 Talin-1 | 6680 |
| P61981_C97 | YWHAG 14-3-3 protein gamma | 6681 |
| Q15020_C341 | SART3 Squamous cell carcinoma | 6682 |
| | antigen recognized by T-cells 3 | |
| O60711_C199 | LPXN Leupaxin | 6683 |
| Q9ULT8_C2415 | HECTD1 E3 ubiquitin-protein ligase | 6684 |
| | HECTD1 | |
| Q99757_C90 | TXN2 Thioredoxin, mitochondrial | 6685 |
| Q99757_C93 | TXN2 Thioredoxin, mitochondrial | 6686 |
| Q13363_C134 | CTBP1 C-terminal-binding protein 1 | 6687 |
| O95571_C34 | ETHE1 Protein ETHE1, mitochondrial | 6688 |
| P31040_C311 | SDHA Succinate dehydrogenase | 6689 |
| P29466_C285 | CASP1 Caspase-1 | 6690 |
| Q9Y4F9_C35 | FAM65B Protein FAM65B | 6691 |
| P35754_C79 | GLRX Glutaredoxin-1 | 6692 |
| Q15149_C950 | PLEC Plectin | 6693 |
| Q13642_C71 | FHL1 Four and a half LIM domains | 6694 |
| | protein 1 | |
| Q00610_C753 | CLTC Clathrin heavy chain 1 | 6695 |
| Q15365_C293 | PCBP1 Poly(rC)-binding protein 1 | 6696 |
| P39023_C253 | RPL3 60S ribosomal protein L3 | 6697 |
| Q96QR8_C238 | PURB Transcriptional activator protein | 6698 |
| | Pur-beta | |
| P29590_C60 | PML Protein PML | 6699 |
| P07814_C1497 | EPRS Bifunctional glutamate/proline-- | 6700 |
| | tRNA ligase | |
| Q9BYW2_C667 | SETD2 Histone-lysine N- | 6701 |
| | methyltransferase SETD2 | |
| Q13469_C569 | NFATC2 Nuclear factor of activated T- | 6702 |
| | cells, cytoplasmic 2 | |
| P31040_C305 | SDHA Succinate dehydrogenase | 6703 |
| P40939_C550 | HADHA Trifunctional enzyme subunit | 6704 |
| | alpha, mitochondrial | |
| P68104_C411 | EEF1A1 Elongation factor 1-alpha 1 | 6705 |
| Q15365_C158 | PCBP1 Poly(rC)-binding protein 1 | 6706 |
| P26599_C250 | PTBP1 Polypyrimidine tract-binding | 6707 |
| | protein 1 | |
| P26599_C251 | PTBP1 Polypyrimidine tract-binding | 6708 |
| | protein 1 | |
| P10768_C176 | ESD S-formylglutathione hydrolase | 6709 |
| P10768_C181 | ESD S-formylglutathione hydrolase | 6710 |
| P30084_C62 | ECHS1 Enoyl-CoA hydratase, | 6711 |
| | mitochondrial | |
| Q15149_C3336 | PLEC Plectin | 6712 |
| P51610_C149 | HCFC1 Host cell factor 1 | 6713 |
| Q9BUJ2_C291 | HNRNPUL1 Heterogeneous nuclear | 6714 |
| | ribonucleoprotein U-like protein | |
| P11413_C385 | G6PD Glucose-6-phosphate 1- | 6715 |
| | dehydrogenase | |
| P00488_C410 | F13A1 Coagulation factor XIIIA chain | 6716 |
| Q9H8H3_C79 | METTL7A Methyltransferase-like | 6717 |
| | protein 7A | |
| Q9NZ08_C498 | ERAP1 Endoplasmic reticulum | 6718 |
| | aminopeptidase 1 | |
| O00303_C256 | EIF3F Eukaryotic translation initiation | 6719 |
| | factor 3 subunit | |
| O75643_C1127 | SNRNP200 U5 small nuclear | 6720 |
| | ribonucleoprotein 200 kDa helicas | |
| Q5T4S7_C1274 | UBR4 E3 ubiquitin-protein ligase | 6721 |
| | UBR4 | |
| Q14676_C2049 | MDC1 Mediator of DNA damage | 6722 |
| | checkpoint protein 1 | |
| Q9Y2X3_C205 | NOP58 Nucleolar protein 58 | 6723 |
| P31930_C453 | UQCRC1 Cytochrome b-c1 complex | 6724 |
| | subunit 1, mitochondrial | |
| Q15019_C111 | SEPT2 Septin-2 | 6725 |
| Q15149_C992 | PLEC Plectin | 6726 |
| Q29RF7_C1079 | PDS5A Sister chromatid cohesion | 6727 |
| | protein PDS5 homolog A | |
| Q6P2E9_C976 | EDC4 Enhancer of mRNA-decapping | 6728 |
| | protein 4 | |
| O00329_C474 | PIK3CD Phosphatidylinositol 4,5- | 6729 |
| | bisphosphate 3-kinase catalytic subunit | |
| | delta isoform | |
| P56192_C12 | MARS Methionine--tRNA ligase, | 6730 |
| | cytoplasmic | |
| P28838_C335 | LAP3 Cytosol aminopeptidase | 6731 |
| P30876_C1093 | POLR2B DNA-directed RNA | 6732 |
| | polymerase II subunit RPB2 | |
| P27635_C195 | RPL10 60S ribosomal protein L10 | 6733 |
| Q9Y490_C1392 | TLN1 Talin-1 | 6734 |
| P55884_C384 | EIF3B Eukaryotic translation initiation | 6735 |
| | factor 3 subunit | |
| Q9UGI8_C46 | TES Testin | 6736 |
| P61978_C184 | HNRNPK Heterogeneous nuclear | 6737 |
| | ribonucleoprotein K | |
| P31943_C122 | HNRNPH1 Heterogeneous nuclear | 6738 |
| | ribonucleoprotein H | |
| Q63HN8_C4348 | RNF213 E3 ubiquitin-protein ligase | 6739 |
| | RNF213 | |
| Q9Y490_C956 | TLN1 Talin-1 | 6740 |
| P08133_C96 | ANXA6 Annexin A6 | 6741 |
| P13667_C206 | PDLA4 Protein disulfide-isomerase A4 | 6742 |
| Q7Z434_C33 | MAVS Mitochondrial antiviral- | 6743 |
| | signaling protein | |
| Q9UKX7_C416 | NUP50 Nuclear pore complex protein | 6744 |
| | Nup50 | |
| Q2M2I8_C319 | AAK1 AP2-associated protein kinase 1 | 6745 |
| Q13547_C100 | HDAC1 Histone deacetylase 1 | 6746 |
| Q13547_C110 | HDAC1 Histone deacetylase 1 | 6747 |
| P13667_C209 | PDIA4 Protein disulfide-isomerase A4 | 6748 |
| P55795_C122 | HNRNPH2 Heterogeneous nuclear | 6749 |
| | ribonucleoprotein H2 | |
| O95544_C79 | NADK NAD kinase | 6750 |
| P62140_C154 | PPP1CB Serine/threonine-protein | 6751 |
| | phosphatase PP1-beta catalytic subunit | |
| P62136_C155 | PPP1CA Serine/threonine-protein | 6752 |
| | phosphatase PP1-alpha catalytic | |
| | subunit | |
| P36873_C155 | PPP1CC Serine/threonine-protein | 6753 |
| | phosphatase PP1-gamma catalytic | |
| | subunit | |
| P53597_C172 | SUCLG1 Succinyl-CoA ligase | 6754 |
| O95071_C730 | UBR5 E3 ubiquitin-protein ligase | 6755 |
| | UBR5 | |
| P22314_C632 | UBA1 Ubiquitin-like modifier- | 6756 |
| | activating enzyme 1 | |
| Q9UBT2_C173 | UBA2 SUMO-activating enzyme | 6757 |
| | subunit 2 | |
| P55072_C572 | VCP Transitional endoplasmic | 6758 |
| | reticulum ATPase | |
| O00329_C90 | PIK3CD Phosphatidylinositol 4,5- | 6759 |
| | bisphosphate 3-kinase catalytic subunit | |
| | delta isoform | |
| Q9UGI8_C238 | TES Testin | 6760 |
| Q14204_C2142 | DYNC1H1 Cytoplasmic dynein 1 | 6761 |
| | heavy chain 1 | |
| Q9Y692_C113 | GMEB1 Glucocorticoid modulatory | 6762 |
| | element-binding protein | |
| Q9UKD1_C110 | GMEB2 Glucocorticoid modulatory | 6763 |
| | element-binding protein | |
| P43686_C210 | PSMC4 26S protease regulatory | 6764 |
| | subunit 6B | |
| O95232_C58 | LUC7L3 Luc7-like protein 3 | 6765 |
| P27695_C99 | APEX1 DNA-(apurinic or apyrimidinic | 6766 |
| | site) lyase | |
| P23743_C432 | DGKA Diacylglycerol kinase alpha | 6767 |
| Q08J23_C93 | NSUN2 tRNA (cytosine(34)-C(5))- | 6768 |
| | methyltransferase | |
| Q92616_C1482 | GCN1L1 Translational activator GCN1 | 6769 |
| Q9Y4B6_C1227 | VPRBP Protein VPRBP | 6770 |
| Q9Y6D5_C1450 | ARFGEF2 Brefeldin A-inhibited | 6771 |
| | guanine nucleotide-exchange | |
| Q6F5E8_C698 | RLTPR Leucine-rich repeat-containing | 6772 |
| | protein 16C | |
| P21964_C119 | COMT Catechol O-methyltransferase | 6773 |
| Q15365_C163 | PCBP1 Poly(rC)-binding protein 1 | 6774 |
| O14617_C208 | AP3D1 AP-3 complex subunit delta-1 | 6775 |
| P53597_C181 | SUCLG1 Succinyl-CoA ligase | 6776 |
| P53582_C14 | METAP1 Methionine aminopeptidase 1 | 6777 |
| Q00839_C497 | HNRNPU Heterogeneous nuclear | 6778 |
| | ribonucleoprotein U | |
| Q9Y6W5_C27 | WASF2 Wiskott-Aldrich syndrome | 6779 |
| | protein family member 2 | |
| Q99439_C164 | CNN2 Calponin-2 | 6780 |
| Q9NVG8_C145 | TBC1D13 TBC1 domain family | 6781 |
| | member 13 | |
| P10768_C56 | ESD S-formylglutathione hydrolase | 6782 |
| P31146_C345 | CORO1A Coronin-1A | 6783 |
| O75521_C380 | ECI2 Enoyl-CoA delta isomerase 2, | 6784 |
| | mitochondrial | |
| P13796_C336 | LCP1 Plastin-2 | 6785 |
| Q15149_C4574 | PLEC Plectin | 6786 |
| P98171_C569 | ARHGAP4 Rho GTPase-activating | 6787 |
| | protein 4 | |
| P48059_C272 | LIMS1 LIM and senescent cell antigen- | 6788 |
| | like-containing domain 1 | |
| Q86WV6_C257 | TMEM173 Transmembrane protein | 6789 |
| | 173 | |
| P23743_C707 | DGKA Diacylglycerol kinase alpha | 6790 |
| Q14204_C3573 | DYNC1H1 Cytoplasmic dynein 1 | 6791 |
| | heavy chain 1 | |
| P22061_C95 | PCMT1 Protein-L-isoaspartate(D- | 6792 |
| | aspartate) O-methyltransferase | |
| Q86W42_C35 | THOC6 THO complex subunit 6 | 6793 |
| | homolog | |
| P55072_C535 | VCP Transitional endoplasmic | 6794 |
| | reticulum ATPase | |
| Q07666_C264 | KHDRBS1 KH domain-containing, | 6795 |
| | RNA-binding, signal transduction | |
| | associated 1 | |
| O00139_C334 | KIF2A Kinesin-like protein KIF2A | 6796 |
| P30481_C188 | HLA-B HLA class I histocompatibility | 6797 |
| | antigen, B-44 alpha | |
| P30483_C188 | HLA-B HLA class I histocompatibility | 6798 |
| | antigen, B-45 alpha | |
| Q8WV92_C233 | MITD1 MIT domain-containing protein | 6799 |
| | 1 | |
| P49207_C83 | RPL34 60S ribosomal protein L34 | 6800 |
| P24752_C119 | ACAT1 Acetyl-CoA acetyltransferase, | 6801 |
| | mitochondrial | |
| P24752_C413 | ACAT1 Acetyl-CoA acetyltransferase, | 6802 |
| | mitochondrial | |
| Q14141_C42 | SEPT6 Septin-6 | 6803 |
| O95319_C174 | CELF2 CUGBP Elav-like family | 6804 |
| | member 2 | |
| O15144_C120 | ARPC2 Actin-related protein 2/3 | 6805 |
| | complex subunit 2 | |
| Q9NTK5_C55 | OLA1 Obg-like ATPase 1 | 6806 |
| Q9Y490_C1953 | TLN1 Talin-1 | 6807 |
| P42331_C258 | ARHGAP25 Rho GTPase-activating | 6808 |
| | protein 25 | |
| O75643_C1580 | SNRNP200 U5 small nuclear | 6809 |
| | ribonucleoprotein 200 kDa helicas | |
| P27635_C49 | RPL10 60S ribosomal protein L10 | 6810 |
| P04075_C202 | ALDOA Fructose-bisphosphate | 6811 |
| | aldolase A | |
| Q92616_C2255 | GCN1L1 Translational activator GCN1 | 6812 |
| Q15149_C3299 | PLEC Plectin | 6813 |
| O94776_C209 | MTA2 Metastasis-associated protein | 6814 |
| | MTA2 | |
| O95466_C69 | FMNL1 Formin-like protein 1 | 6815 |
| P09972_C202 | ALDOC Fructose-bisphosphate | 6816 |
| | aldolase C | |
| Q16186_C80 | ADRM1 Proteasomal ubiquitin | 6817 |
| | receptor ADRM1 | |
| Q9Y224_C19 | C14orf166 UPF0568 protein | 6818 |
| | C14orf166 | |
| P23743_C253 | DGKA Diacylglycerol kinase alpha | 6819 |
| Q13464_C29 | ROCK1 Rho-associated protein kinase | 6820 |
| | 1 | |
| O14617_C1133 | AP3D1 AP-3 complex subunit delta-1 | 6821 |
| P05062_C202 | ALDOB Fructose-bisphosphate | 6822 |
| | aldolase B | |
| O95154_C186 | AKR7A3 Aflatoxin B1 aldehyde | 6823 |
| | reductase member 3 | |
| Q8NHP1_C186 | AKR7L Aflatoxin B1 aldehyde | 6824 |
| | reductase member 4 | |
| O43488_C214 | AKR7A2 Aflatoxin B1 aldehyde | 6825 |
| | reductase member 2 | |
| P23246_C431 | SFPQ Splicing factor, proline- and | 6826 |
| | glutamine-rich | |
| O00268_C867 | TAF4 Transcription initiation factor | 6827 |
| | TFTTD subunit 4 | |
| P61158_C12 | ACTR3 Actin-related protein 3 | 6828 |
| P21333_C2601 | FLNA Filamin-A | 6829 |
| O00567_C384 | NOP56 Nucleolar protein 56 | 6830 |
| P46776_C70 | RPL27A 60S ribosomal protein L27a | 6831 |
| P68366_C54 | TUBA4A Tubulin alpha-4A chain | 6832 |
| P68366_C295 | TUBA4A Tubulin alpha-4A chain | 6833 |
| Q71U36_C295 | TUBA1A Tubulin alpha-1A chain | 6834 |
| P62829_C125 | RPL23 60S ribosomal protein L23 | 6835 |
| O95881_C66 | TXNDC12 Thioredoxin domain- | 6836 |
| | containing protein 12 | |
| P50552_C64 | VASP Vasodilator-stimulated | 6837 |
| | phosphoprotein | |
| Q9UJU6_C127 | DBNL Drebrin-like protein | 6838 |
| Q9UNH7_C264 | SNX6 Sorting nexin-6 | 6839 |
| Q8WVJ2_C99 | NUDCD2 NudC domain-containing | 6840 |
| | protein 2 | |
| Q9Y490_C1486 | TLN1 Talin-1 | 6841 |
| Q8WYJ6_C231 | SEPT1 Septin-1 | 6842 |
| Q15149_C317 | PLEC Plectin | 6843 |
| Q15027_C79 | ACAP1 Arf-GAP with coiled-coil, | 6844 |
| | ANK repeat and PH domain 1 | |
| Q9BXJ9_C322 | NAA15 N-alpha-acetyltransferase 15, | 6845 |
| | NatA auxiliary subunit | |
| Q86YV0_C447 | RASAL3 RAS protein activator like-3 | 6846 |
| Q63HN8_C2918 | RNF213 E3 ubiquitin-protein ligase | 6847 |
| | RNF213 | |
| P61158_C8 | ACTR3 Actin-related protein 3 | 6848 |
| Q8WWQ0_C1144 | PHIP PH-interacting protein | 6849 |
| Q9Y490_C1478 | TLN1 Talin-1 | 6850 |
| Q10567_C95 | AP1B1 AP-1 complex subunit beta-1 | 6851 |
| Q13748_C295 | TUBA3D Tubulin alpha-3C/D chain | 6852 |
| Q8N1G4_C224 | LRRC47 Leucine-rich repeat- | 6853 |
| | containing protein 47 | |
| E9PPU0_C611 | EPPK1 Epiplakin | 6854 |
| Q92878_C102 | RAD50 DNA repair protein RAD50 | 6855 |
| Q9UKE5_C202 | TNIK TRAF2 and NCK-interacting | 6856 |
| | protein kinase | |
| Q96FV9_C445 | THOC1 THO complex subunit 1 | 6857 |
| Q92747_C162 | ARPC1A Actin-related protein 2/3 | 6858 |
| | complex subunit 1A | |
| O15143_C162 | ARPC1B Actin-related protein 2/3 | 6859 |
| | complex subunit 1B | |
| P12236_C160 | SLC25A6 ADP/ATP translocase 3 | 6860 |
| P05141_C160 | SLC25A5 ADP/ATP translocase 2 | 6861 |
| Q13315_C2021 | ATM Serine-protein kinase ATM | 6862 |
| P50990_C244 | CCT8 T-complex protein 1 subunit | 6863 |
| | theta | |
| Q00839_C607 | HNRNPU Heterogeneous nuclear | 6864 |
| | ribonucleoprotein U | |
| P10809_C442 | HSPD1 60 kDa heat shock protein, | 6865 |
| | mitochondrial | |
| Q676U5_C145 | ATG16L1 Autophagy-related protein | 6866 |
| | 16-1 | |
| Q86UX7_C235 | FERMT3 Fermitin family homolog 3 | 6867 |
| P21333_C1353 | FLNA Filamin-A | 6868 |
| Q56VL3_C27 | OCIAD2 OCIA domain-containing | 6869 |
| | protein 2 | |
| Q14204_C1977 | DYNC1H1 Cytoplasmic dynein 1 | 6870 |
| | heavy chain 1 | |
| Q9NUV9_C187 | GIMAP4 GTPase IMAP family | 6871 |
| | member 4 | |
| P52272_C114 | HNRNPM Heterogeneous nuclear | 6872 |
| | ribonucleoprotein M | |
| P78527_C3781 | PRKDC DNA-dependent protein | 6873 |
| | kinase catalytic subunit | |
| P39023_C114 | RPL3 60S ribosomal protein L3 | 6874 |
| Q14202_C1326 | ZMYM3 Zinc finger MYM-type | 6875 |
| | protein 3 | |
| O43684_C129 | BUB3 Mitotic checkpoint protein | 6876 |
| | BUB3 | |
| Q8N2I2_C302 | ZNF619 Zinc finger protein 619 | 6877 |
| Q92900_C657 | UPF1 Regulator of nonsense transcripts | 6878 |
| | 1 | |
| Q00839_C389 | HNRNPU Heterogeneous nuclear | 6879 |
| | ribonucleoprotein U | |
| Q00839_C391 | HNRNPU Heterogeneous nuclear | 6880 |
| | ribonucleoprotein U | |
| Q9Y3U8_C48 | RPL36 60S ribosomal protein L36 | 6881 |
| Q7L2J0_C177 | MEPCE 7SK snRNA methylphosphate | 6882 |
| | capping enzyme | |
| Q01518_C93 | CAP1 Adenylyl cyclase-associated | 6883 |
| | protein 1 | |
| P55769_C30 | NHP2L1 NHP2-like protein 1 | 6884 |
| P46776_C144 | RPL27A 60S ribosomal protein L27a | 6885 |
| Q9H3P7_C129 | ACBD3 Golgi resident protein GCP60 | 6886 |
| Q9NQR4_C153 | NIT2 Omega-amidase NIT2 | 6887 |
| O60216_C392 | RAD21 Double-strand-break repair | 6888 |
| | protein rad21 homolog | |
| P13639_C67 | EEF2 Elongation factor 2 | 6889 |
| Q8WWP7_C76 | GIMAP1 GTPase IMAP family | 6890 |
| | member 1 | |
| Q9NTI5_C1069 | PDS5B Sister chromatid cohesion | 6891 |
| | protein PDS5 homolog B | |
| P41091_C105 | EIF2S3 Eukaryotic translation initiation | 6892 |
| | factor 2 subunit | |
| Q9Y2I8_C417 | WDR37 WD repeat-containing protein | 6893 |
| | 37 | |
| P52566_C76 | ARHGDIB Rho GDP-dissociation | 6894 |
| | inhibitor 2 | |
| Q9BSJ8_C522 | ESYT1 Extended synaptotagmin-1 | 6895 |
| Q8N163_C238 | KIAA1967 DBIRD complex subunit | 6896 |
| | KIAA1967 | |
| Q92835_C672 | INPP5D Phosphatidylinositol 3,4,5- | 6897 |
| | trisphosphate 5-phosphatase | |
| Q8NB90_C672 | SPATA5 Spermatogenesis-associated | 6898 |
| | protein 5 | |
| Q92878_C157 | RAD50 DNA repair protein RAD50 | 6899 |
| Q15370_C89 | TCEB2 Transcription elongation factor | 6900 |
| | B polypeptide 2 | |
| Q9UNF1_C516 | MAGED2 Melanoma-associated | 6901 |
| | antigen D2 | |
| Q08AF3_C48 | SLFN5 Schlafen family member 5 | 6902 |
| Q01082_C1900 | SPTBN1 Spectrin beta chain, non- | 6903 |
| | erythrocytic 1 | |
| Q8IUI8_C154 | CRLF3 Cytokine receptor-like factor 3 | 6904 |
| P57737_C505 | CORO7 Coronin-7 | 6905 |
| O95336_C237 | PGLS 6-phosphogluconolactonase | 6906 |
| O15372_C327 | EIF3H Eukaryotic translation initiation | 6907 |
| | factor 3 subunit | |
| Q63HN8_C3609 | RNF213 E3 ubiquitin-protein ligase | 6908 |
| | RNF213 | |
| P00367_C327 | GLUD1 Glutamate dehydrogenase 1, | 6909 |
| | mitochondrial | |
| Q15185_C76 | PTGES3 Prostaglandin E synthase 3 | 6910 |
| Q86UX7_C439 | FERMT3 Fermitin family homolog 3 | 6911 |
| A6NHR9_C458 | SMCHD1 Structural maintenance of | 6912 |
| | chromosomes flexible hinge domain- | |
| | containing protein 1 | |
| P49448_C327 | GLUD2 Glutamate dehydrogenase 2, | 6913 |
| | mitochondrial | |
| Q15052_C530 | ARHGEF6 Rho guanine nucleotide | 6914 |
| | exchange factor 6 | |
| H3BQZ7_C538 | Uncharacterized protein | 6915 |
| Q1KMD3_C538 | HNRNPUL2 Heterogeneous nuclear | 6916 |
| | ribonucleoprotein U-like protein 2 | |
| P11413_C13 | G6PD Glucose-6-phosphate 1- | 6917 |
| | dehydrogenase | |
| P57737_C34 | CORO7 Coronin-7 | 6918 |
| P55735_C187 | SEC13 Protein SEC13 homolog | 6919 |
| P68366_C315 | TUBA4A Tubulin alpha-4A chain | 6920 |
| P68366_C316 | TUBA4A Tubulin alpha-4A chain | 6921 |
| Q71U36_C315 | TUBA1A Tubulin alpha-1A chain | 6922 |
| Q71U36_C316 | TUBA1A Tubulin alpha-1A chain | 6923 |
| P31146_C195 | CORO1A Coronin-1A | 6924 |
| P68371_C354 | TUBB4B Tubulin beta-4B chain | 6925 |
| P07437_C354 | TUBB Tubulin beta chain | 6926 |
| P29350_C102 | PTPN6 Tyrosine-protein phosphatase | 6927 |
| | non-receptor type 6 | |
| P21333_C1260 | FLNA Filamin-A | 6928 |
| P08670_C328 | VIM Vimentin | 6929 |
| P42167_C363 | TMPO Lamina-associated polypeptide | 6930 |
| | 2, isoforms beta/gamma | |
| Q9H0C8_C325 | ILKAP Integrin-linked kinase- | 6931 |
| | associated serine/threonine | |
| Q66K74_C342 | MAP1S Microtubule-associated protein | 6932 |
| | 1S | |
| Q7KZF4_C560 | SND1 Staphylococcal nuclease | 6933 |
| | domain-containing protein | |
| Q9P1F3_C39 | ABRACL Costars family protein | 6934 |
| | ABRACL | |
| P36507_C211 | MAP2K2 Dual specificity mitogen- | 6935 |
| | activated protein kinase | |
| Q02750_C207 | MAP2K1 Dual specificity mitogen- | 6936 |
| | activated protein kinase | |
| Q8NF50_C685 | DOCK8 Dedicator of cytokinesis | 6937 |
| | protein 8 | |
| Q9BVA1_C354 | TUBB2B Tubulin beta-2B chain | 6938 |
| P36578_C125 | RPL4 60S ribosomal protein L4 | 6939 |
| Q8TD19_C890 | NEK9 Serine/threonine-protein kinase | 6940 |
| | Nek9 | |
| A6NHR9_C1286 | SMCHD1 Structural maintenance of | 6941 |
| | chromosomes flexible hinge domain- | |
| | containing protein 1 | |
| Q9BSD7_C110 | NTPCR Cancer-related nucleoside- | 6942 |
| | triphosphatase | |
| Q14573_C1638 | ITPR3 Inositol 1,4,5-trisphosphate | 6943 |
| | receptor type 3 | |
| Q12874_C274 | SF3A3 Splicing factor 3A subunit 3 | 6944 |
| Q13045_C337 | FLII Protein flightless-1 homolog | 6945 |
| Q8IY67_C255 | RAVER1 Ribonucleoprotein PTB- | 6946 |
| | binding 1 | |
| P13639_C728 | EEF2 Elongation factor 2 | 6947 |
| P53990_C125 | IST1 IST1 homolog | 6948 |
| P13796_C618 | LCP1 Plastin-2 | 6949 |
| Q9BWM7_C193 | SFXN3 Sideroflexin-3 | 6950 |
| Q9H9B4_C190 | SFXN1 Sideroflexin-1 | 6951 |
| P31146_C192 | CORO1A Coronin-1A | 6952 |
| Q15024_C85 | EXOSC7 Exosome complex | 6953 |
| | component RRP42 | |
| Q8N4C8_C202 | MINK1 Misshapen-like kinase 1 | 6954 |
| O95819_C202 | MAP4K4 Mitogen-activated protein | 6955 |
| | kinase kinase kinase kinase | |
| P13798_C30 | APEH Acylamino-acid-releasing | 6956 |
| | enzyme | |
| P35579_C172 | MYH9 Myosin-9 | 6957 |
| P30626_C194 | SRI Sorcin | 6958 |
| O15371_C19 | EIF3D Eukaryotic translation initiation | 6959 |
| | factor 3 subunit | |
| O00567_C211 | NOP56 Nucleolar protein 56 | 6960 |
| Q9UGI8_C196 | TES Testin | 6961 |
| P67936_C247 | TPM4 Tropomyosin alpha-4 chain | 6962 |
| P45954_C175 | ACADSB Short/branched chain | 6963 |
| | specific acyl-CoA dehydrogenase | |
| Q14566_C301 | MCM6 DNA replication licensing | 6964 |
| | factor MCM6 | |
| P50990_C149 | CCT8 T-complex protein 1 subunit | 6965 |
| | theta | |
| Q08945_C200 | SSRP1 FACT complex subunit SSRP1 | 6966 |
| P45985_C246 | MAP2K4 Dual specificity mitogen- | 6967 |
| | activated protein kinase | |
| P21333_C2582 | FLNA Filamin-A | 6968 |
| P29590_C213 | PML Protein PML | 6969 |
| P13489_C30 | RNH1 Ribonuclease inhibitor | 6970 |
| Q13315_C2770 | ATM Serine-protein kinase ATM | 6971 |
| Q9NR56_C34 | MBNL1 Muscleblind-like protein 1 | 6972 |
| Q08AF3_C114 | SLFN5 Schlafen family member 5 | 6973 |
| Q53EL6_C150 | PDCD4 Programmed cell death protein | 6974 |
| | 4 | |
| Q9NPJ6_C162 | MED4 Mediator of RNA polymerase II | 6975 |
| | transcription subunit | |
| P25398_C92 | RPS12 40S ribosomal protein S12 | 6976 |
| P57737_C303 | CORO7 Coronin-7 | 6977 |
| Q96RL1_C283 | UIMC1 BRCA1-A complex subunit | 6978 |
| | RAP80 | |
| P24928_C1245 | POLR2A DNA-directed RNA | 6979 |
| | polymerase II subunit RPB1 | |
| P17858_C89 | PFKL 6-phosphofructokinase, liver | 6980 |
| | type | |
| P50995_C226 | ANXA11 Annexin A11 | 6981 |
| Q7Z460_C1428 | CLASP1 CLIP-associating protein 1 | 6982 |
| O75122_C1184 | CLASP2 CLIP-associating protein 2 | 6983 |
| Q14008_C1344 | CKAP5 Cytoskeleton-associated | 6984 |
| | protein 5 | |
| Q14008_C1360 | CKAP5 Cytoskeleton-associated | 6985 |
| | protein 5 | |
| P16455_C145 | MGMT Methylated-DNA--protein- | 6986 |
| | cysteine methyltransferase | |
| O14976_C190 | GAK Cyclin-G-associated kinase | 6987 |
| Q14103_C226 | HNRNPD Heterogeneous nuclear | 6988 |
| | ribonucleoprotein D0 | |
| P11586_C918 | MTHFD1 C-1-tetrahydrofolate | 6989 |
| | synthase, cytoplasmic | |
| Q14008_C1350 | CKAP5 Cytoskeleton-associated | 6990 |
| | protein 5 | |
| P42704_C484 | LRPPRC Leucine-rich PPR motif- | 6991 |
| | containing protein, mitochondrial | |
| P50990_C148 | CCT8 T-complex protein 1 subunit | 6992 |
| | theta | |
| P33992_C197 | MCM5 DNA replication licensing | 6993 |
| | factor MCM5 | |
| O43143_C190 | DHX15 Putative pre-mRNA-splicing | 6994 |
| | factor ATP-dependent RN | |
| P07814_C381 | EPRS Bifunctional glutamate/proline-- | 6995 |
| | tRNA ligase | |
| P24928_C13 | POLR2A DNA-directed RNA | 6996 |
| | polymerase II subunit RPB1 | |
| P24928_C1287 | POLR2A DNA-directed RNA | 6997 |
| | polymerase II subunit RPB1 | |
| P42858_C664 | HTT Huntingtin | 6998 |
| Q13155_C23 | AIMP2 Aminoacyl tRNA synthase | 6999 |
| | complex-interacting multif | |
| Q14203_C1252 | DCTN1 Dynactin subunit 1 | 7000 |
| P68366_C347 | TUBA4A Tubulin alpha-4A chain | 7001 |
| Q00839_C648 | HNRNPU Heterogeneous nuclear | 7002 |
| | ribonucleoprotein U | |
| Q92616_C2558 | GCN1L1 Translational activator GCN1 | 7003 |
| Q86SX6_C67 | GLRX5 Glutaredoxin-related protein 5, | 7004 |
| | mitochondrial | |
| P24752_C196 | ACAT1 Acetyl-CoA acetyltransferase, | 7005 |
| | mitochondrial | |
| O75150_C950 | RNF40 E3 ubiquitin-protein ligase | 7006 |
| | BRE1B | |
| P62979_C149 | RPS27A Ubiquitin-40S ribosomal | 7007 |
| | protein S27a | |
| Q9Y3A3_C134 | MOB4 MOB-like protein phocein | 7008 |
| Q8IUI8_C95 | CRLF3 Cytokine receptor-like factor 3 | 7009 |
| Q96SK2_C295 | TMEM209 Transmembrane protein | 7010 |
| | 209 | |
| P21964_C238 | COMT Catechol O-methyltransferase | 7011 |
| P62753_C100 | RPS6 40S ribosomal protein S6 | 7012 |
| Q9Y3Z3_C522 | SAMHD1 SAM domain and HD | 7013 |
| | domain-containing protein 1 | |
| Q12802_C2142 | AKAP13 A-kinase anchor protein 13 | 7014 |
| Q14697_C502 | GANAB Neutral alpha-glucosidase AB | 7015 |
| P62826_C112 | RAN GTP-binding nuclear protein Ran | 7016 |
| O15379_C94 | HDAC3 Histone deacetylase 3 | 7017 |
| P13010_C296 | XRCC5 X-ray repair cross- | 7018 |
| | complementing protein 5 | |
| Q9Y5Y2_C54 | NUBP2 Cytosolic Fe—S cluster | 7019 |
| | assembly factor NUBP2 | |
| Q9BRJ7_C171 | NUDT16L1 Protein syndesmos | 7020 |
| P35579_C91 | MYH9 Myosin-9 | 7021 |
| P46940_C494 | IQGAP1 Ras GTPase-activating-like | 7022 |
| | protein IQGAP1 | |
| Q6F5E8_C823 | RLTPR Leucine-rich repeat-containing | 7023 |
| | protein 16C | |
| Q8IX12_C465 | CCAR1 Cell division cycle and | 7024 |
| | apoptosis regulator protein 1 | |
| Q6F5E8_C582 | RLTPR Leucine-rich repeat-containing | 7025 |
| | protein 16C | |
| Q13148_C198 | TARDBP TAR DNA-binding protein | 7026 |
| | 43 | |
| Q9Y6N5_C337 | SQRDL Sulfide:quinone | 7027 |
| | oxidoreductase, mitochondrial | |
| Q02880_C204 | TOP2B DNA topoisomerase 2-beta | 7028 |
| P62826_C120 | RAN GTP-binding nuclear protein Ran | 7029 |
| Q8NBN7_C30 | RDH13 Retinol dehydrogenase 13 | 7030 |
| P62979_C145 | RPS27A Ubiquitin-40S ribosomal | 7031 |
| | protein S27a | |
| P41250_C471 | GARS Glycine--tRNA ligase | 7032 |
| P46940_C781 | IQGAP1 Ras GTPase-activating-like | 7033 |
| | protein IQGAP1 | |
| P42765_C92 | ACAA2 3-ketoacyl-CoA thiolase, | 7034 |
| | mitochondrial | |
| Q8WWP7_C186 | GIMAP1 GTPase IMAP family | 7035 |
| | member 1 | |
| P49207_C49 | RPL34 60S ribosomal protein L34 | 7036 |
| Q8IUI8_C313 | CRLF3 Cytokine receptor-like factor 3 | 7037 |
| P17655_C301 | CAPN2 Calpain-2 catalytic subunit | 7038 |
| Q13526_C113 | PIN1 Peptidyl-prolyl cis-trans | 7039 |
| | isomerase NIMA-interacti | |
| O60313_C375 | OPA1 Dynamin-like 120 kDa protein, | 7040 |
| | mitochondrial | |
| P68371_C303 | TUBB4B Tubulin beta-4B chain | 7041 |
| P07437_C303 | TUBB Tubulin beta chain | 7042 |
| Q9UPN7_C37 | PPP6R1 Serine/threonine-protein | 7043 |
| | phosphatase 6 regulatory | |
| Q96SW2_C188 | CRBN Protein cereblon | 7044 |
| O14980_C1070 | XPO1 Exportin-1 | 7045 |
| Q15366_C109 | PCBP2 Poly(rC)-binding protein 2 | 7046 |
| P30154_C402 | PPP2R1B Serine/threonine-protein | 7047 |
| | phosphatase 2A 65 kDa regulatory | |
| | subunit A beta isoform | |
| P30153_C390 | PPP2R1A Serine/threonine-protein | 7048 |
| | phosphatase 2A 65 kDa regulatory | |
| | subunit A alpha isoform | |
| P07195_C294 | LDHB L-lactate dehydrogenase B | 7049 |
| | chain | |
| P31943_C22 | HNRNPH1 Heterogeneous nuclear | 7050 |
| | ribonucleoprotein H | |
| P53582_C40 | METAP1 Methionine aminopeptidase 1 | 7051 |
| P61221_C88 | ABCE1 ATP-binding cassette sub- | 7052 |
| | family E member 1 | |
| P49207_C46 | RPL34 60S ribosomal protein L34 | 7053 |
| P13639_C651 | EEF2 Elongation factor 2 | 7054 |
| Q9BVA1_C303 | TUBB2B Tubulin beta-2B chain | 7055 |
| Q01082_C604 | SPTBN1 Spectrin beta chain, non- | 7056 |
| | erythrocytic 1 | |
| Q9Y490_C1045 | TLN1 Talin-1 | 7057 |
| Q15366_C158 | PCBP2 Poly(rC)-binding protein 2 | 7058 |
| O95573_C450 | ACSL3 Long-chain-fatty-acid--CoA | 7059 |
| | ligase 3 | |
| Q9BT78_C378 | COPS4 COP9 signalosome complex | 7060 |
| | subunit 4 | |
| Q53EL6_C288 | PDCD4 Programmed cell death protein | 7061 |
| | 4 | |
| Q92841_C319 | DDX17 Probable ATP-dependent RNA | 7062 |
| | helicase DDX17 | |
| P49411_C127 | TUFM Elongation factor Tu, | 7063 |
| | mitochondrial | |
| Q96P65_C201 | QRFPR Pyroglutamylated RFamide | 7064 |
| | peptide receptor | |
| P46734_C227 | MAP2K3 Dual specificity mitogen- | 7065 |
| | activated protein kinase | |
| P48643_C253 | CCT5 T-complex protein 1 subunit | 7066 |
| | epsilon | |
| Q96GX9_C147 | APIP Probable methylthioribulose-1- | 7067 |
| | phosphate dehydratase | |
| O00567_C112 | NOP56 Nucleolar protein 56 | 7068 |
| Q92835_C819 | INPP5D Phosphatidylinositol 3,4,5- | 7069 |
| | trisphosphate 5-phospha | |
| P21333_C623 | FLNA Filamin-A | 7070 |
| Q13509_C303 | TUBB3 Tubulin beta-3 chain | 7071 |
| O60759_C210 | CYTIP Cytohesin-interacting protein | 7072 |
| Q96P65_C200 | QRFPR Pyroglutamylated RFamide | 7073 |
| | peptide receptor | |
| Q9BUF5_C303 | TUBB6 Tubulin beta-6 chain | 7074 |
| O75369_C455 | FLNB Filamin-B | 7075 |
| Q9UEY8_C245 | ADD3 Gamma-adducin | 7076 |
| P06239_C465 | LCK Tyrosine-protein kinase Lck | 7077 |
| P60953_C157 | CDC42 Cell division control protein 42 | 7078 |
| | homolog | |
| P11586_C863 | MTHFD1 C-1-tetrahydrofolate | 7079 |
| | synthase, cytoplasmic | |
| Q71U36_C347 | TUBA1A Tubulin alpha-1A chain | 7080 |
| P20073_C363 | ANXA7 Annexin A7 | 7081 |
| Q8WYJ6_C293 | SEPT1 Septin-1 | 7082 |
| P04406_C152 | GAPDH Glyceraldehyde-3-phosphate | 7083 |
| | dehydrogenase | |
| P49748_C237 | ACADVL Very long-chain specific | 7084 |
| | acyl-CoA dehydrogenase, | |
| | mitochondrial | |
| P53618_C635 | COPB1 Coatomer subunit beta | 7085 |
| Q14669_C1538 | TRIP12 E3 ubiquitin-protein ligase | 7086 |
| | TRIP12 | |
| Q8WWP7_C98 | GIMAP1 GTPase IMAP family | 7087 |
| | member 1 | |
| P49840_C262 | GSK3A Glycogen synthase kinase-3 | 7088 |
| | alpha | |
| Q9UKV3_C1223 | ACIN1 Apoptotic chromatin | 7089 |
| | condensation inducer in the nucleus | |
| Q8N5W9_C86 | FAM101B Protein FAM101B | 7090 |
| P68371_C201 | TUBB4B Tubulin beta-4B chain | 7091 |
| P68371_C211 | TUBB4B Tubulin beta-4B chain | 7092 |
| Q9BVA1_C201 | TUBB2B Tubulin beta-2B chain | 7093 |
| Q9BVA1_C211 | TUBB2B Tubulin beta-2B chain | 7094 |
| P07437_C201 | TUBB Tubulin beta chain | 7095 |
| P07437_C211 | TUBB Tubulin beta chain | 7096 |
| Q8IV04_C305 | TBC1D10C Carabin | 7097 |
| O60664_C341 | PLIN3 Perilipin-3 | 7098 |
| P53384_C277 | NUBP1 Cytosolic Fe—S cluster | 7099 |
| | assembly factor NUBP1 | |
| Q99439_C240 | CNN2 Calponin-2 | 7100 |
| P49841_C199 | GSK3B Glycogen synthase kinase-3 | 7101 |
| | beta | |
| P04406_C156 | GAPDH Glyceraldehyde-3-phosphate | 7102 |
| | dehydrogenase | |
| O95758_C55 | PTBP3 Polypyrimidine tract-binding | 7103 |
| | protein 3 | |
| Q9NY65_C347 | TUBA8 Tubulin alpha-8 chain | 7104 |
| Q13748_C347 | TUBA3D Tubulin alpha-3C/D chain | 7105 |
| Q86WN1_C449 | FCHSD1 FCH and double SH3 | 7106 |
| | domains protein 1 | |
| Q92619_C413 | HMHA1 Minor histocompatibility | 7107 |
| | protein HA-1 | |
| Q93009_C223 | USP7 Ubiquitin carboxyl-terminal | 7108 |
| | hydrolase 7 | |
| Q9BUF5_C201 | TUBB6 Tubulin beta-6 chain | 7109 |
| Q9BUF5_C211 | TUBB6 Tubulin beta-6 chain | 7110 |
| Q70J99_C136 | UNC13D Protein unc-13 homolog D | 7111 |
| O95486_C704 | SEC24A Protein transport protein | 7112 |
| | Sec24A | |
| P27987_C693 | ITPKB Inositol-trisphosphate 3-kinase | 7113 |
| | B | |
| P84103_C6 | SRSF3 Serine/arginine-rich splicing | 7114 |
| | factor 3 | |
| P24752_C126 | ACAT1 Acetyl-CoA acetyltransferase, | 7115 |
| | mitochondrial | |
| O00299_C223 | CLIC1 Chloride intracellular channel | 7116 |
| | protein 1 | |
| P68366_C376 | TUBA4A Tubulin alpha-4A chain | 7117 |
| Q71U36_C376 | TUBA1A Tubulin alpha-1A chain | 7118 |
| Q15365_C109 | PCBP1 Poly(rC)-binding protein 1 | 7119 |
| P62280_C60 | RPS11 40S ribosomal protein S11 | 7120 |
| P62829_C28 | RPL23 60S ribosomal protein L23 | 7121 |
| P05388_C119 | RPLP0 60S acidic ribosomal protein P0 | 7122 |
| P62937_C161 | PPIA Peptidyl-prolyl cis-trans | 7123 |
| | isomerase A | |
| P28062_C120 | PSMB8 Proteasome subunit beta type-8 | 7124 |
| P28062_C124 | PSMB8 Proteasome subunit beta type-8 | 7125 |
| Q9Y228_C256 | TRAF3IP3 TRAF3-interacting JNK- | 7126 |
| | activating modulator | |
| Q9BY49_C191 | PECR Peroxisomal trans-2-enoyl-CoA | 7127 |
| | reductase | |
| P30154_C306 | PPP2R1B Serine/threonine-protein | 7128 |
| | phosphatase 2A 65 kDa regulatory | |
| | subunit A beta isoform | |
| P30153_C294 | PPP2R1A Serine/threonine-protein | 7129 |
| | phosphatase 2A 65 kDa regulatory | |
| | subunit A alpha isoform | |
| P52564_C196 | MAP2K6 Dual specificity mitogen- | 7130 |
| | activated protein kinase | |
| P46734_C207 | MAP2K3 Dual specificity mitogen- | 7131 |
| | activated protein kinase | |
| Q9NYL9_C150 | TMOD3 Tropomodulin-3 | 7132 |
| O75175_C600 | CNOT3 CCR4-NOT transcription | 7133 |
| | complex subunit 3 | |
| Q9H0D6_C736 | XRN2 5-3 exoribonuclease 2 | 7134 |
| Q8IV53_C52 | DENND1C DENN domain-containing | 7135 |
| | protein 1C | |
| P26358_C1478 | DNMT1 DNA (cytosine-5)- | 7136 |
| | methyltransferase 1 | |
| Q7RTV0_C40 | PHF5A PHD finger-like domain- | 7137 |
| | containing protein 5A | |
| P51570_C182 | GALK1 Galactokinase | 7138 |
| Q9Y490_C1939 | TLN1 Talin-1 | 7139 |
| P49848_C460 | TAF6 Transcription initiation factor | 7140 |
| | TFIID subunit 6 | |
| Q9NY65_C376 | TUBA8 Tubulin alpha-8 chain | 7141 |
| Q13748_C376 | TUBA3D Tubulin alpha-3C/D chain | 7142 |
| Q9Y512_C457 | SAMM50 Sorting and assembly | 7143 |
| | machinery component 50 homolog | |
| P40939_C470 | HADHA Trifunctional enzyme subunit | 7144 |
| | alpha, mitochondrial | |
| Q6VY07_C732 | PACS1 Phosphofurin acidic cluster | 7145 |
| | sorting protein 1 | |
| Q9NWV8_C222 | BABAM1 BRISC and BRCA1-A | 7146 |
| | complex member 1 | |
| Q7KZF4_C152 | SND1 Staphylococcal nuclease | 7147 |
| | domain-containing protein | |
| P12814_C480 | ACTN1 Alpha-actinin-1 | 7148 |
| P07900_C597 | HSP90AA1 Heat shock protein HSP | 7149 |
| | 90-alpha | |
| P07900_C598 | HSP90AA1 Heat shock protein HSP | 7150 |
| | 90-alpha | |
| H0Y2S0_C31 | Uncharacterized protein | 7151 |
| Q6UB35_C906 | MTHFD1L Monofunctional C1- | 7152 |
| | tetrahydrofolate synthase, | |
| | mitochondrial | |
| Q71UM5_C77 | RPS27L 40S ribosomal protein S27- | 7153 |
| | like | |
| P42677_C77 | RPS27 40S ribosomal protein S27 | 7154 |
| Q08211_C1029 | DHX9 ATP-dependent RNA helicase A | 7155 |
| O15533_C115 | TAPBP Tapasin | 7156 |
| P29590_C389 | PML Protein PML | 7157 |
| P62937_C62 | PPIA Peptidyl-prolyl cis-trans | 7158 |
| | isomerase A | |
| Q14204_C1999 | DYNC1H1 Cytoplasmic dynein 1 | 7159 |
| | heavy chain 1 | |
| Q13561_C240 | DCTN2 Dynactin subunit 2 | 7160 |
| Q13561_C256 | DCTN2 Dynactin subunit 2 | 7161 |
| H3BQZ7_C602 | Uncharacterized protein | 7162 |
| Q1KMD3_C602 | HNRNPUL2 Heterogeneous nuclear | 7163 |
| | ribonucleoprotein U-like protein 2 | |
| P61247_C96 | RPS3A 40S ribosomal protein S3a | 7164 |
| P68871_C94 | HBB Hemoglobin subunit beta | 7165 |
| Q9UHD8_C375 | SEPT9 Septin-9 | 7166 |
| Q8NFW8_C394 | CMAS N-acylneuraminate | 7167 |
| | cytidylyltransferase | |
| P51878_C315 | CASP5 Caspase-5 | 7168 |
| P49662_C258 | CASP4 Caspase-4 | 7169 |
| O75083_C170 | WDR1 WD repeat-containing protein 1 | 7170 |
| P55884_C515 | EIF3B Eukaryotic translation initiation | 7171 |
| | factor 3 subunit | |
| Q9UKX7_C181 | NUP50 Nuclear pore complex protein | 7172 |
| | Nup50 | |
| Q13185_C69 | CBX3 Chromobox protein homolog 3 | 7173 |
| O00299_C59 | CLIC1 Chloride intracellular channel | 7174 |
| | protein 1 | |
| Q06587_C69 | RING1 E3 ubiquitin-protein ligase | 7175 |
| | RING1 | |
| Q14204_C3033 | DYNC1H1 Cytoplasmic dynein 1 | 7176 |
| | heavy chain 1 | |
| Q13045_C241 | FLII Protein flightless-1 homolog | 7177 |
| P48643_C181 | CCT5 T-complex protein 1 subunit | 7178 |
| | epsilon | |
| Q04726_C528 | TLE3 Transducin-like enhancer protein | 7179 |
| | 3 | |
| Q15149_C3493 | PLEC Plectin | 7180 |
| O00299_C24 | CLIC1 Chloride intracellular channel | 7181 |
| | protein 1 | |
| P35579_C694 | MYH9 Myosin-9 | 7182 |
| Q9Y490_C732 | TLN1 Talin-1 | 7183 |
| Q8WWP7_C66 | GIMAP1 GTPase IMAP family | 7184 |
| | member 1 | |
| P49721_C91 | PSMB2 Proteasome subunit beta type-2 | 7185 |
| P52272_C653 | HNRNPM Heterogeneous nuclear | 7186 |
| | ribonucleoprotein M | |
| Q9UK45_C85 | LSM7 U6 snRNA-associated Sm-like | 7187 |
| | protein LSm7 | |
| P21333_C2199 | FLNA Filamin-A | 7188 |
| P22087_C268 | FBL rRNA 2-O-methyltransferase | 7189 |
| | fibrillarin | |
| Q99714_C214 | HSD17B10 3-hydroxyacyl-CoA | 7190 |
| | dehydrogenase type-2 | |
| Q96F86_C413 | EDC3 Enhancer of mRNA-decapping | 7191 |
| | protein 3 | |
| P53621_C975 | COPA Coatomer subunit alpha | 7192 |
| Q96S55_C272 | WRNIP1 ATPase WRNIP1 | 7193 |
| P15498_C652 | VAV1 Proto-oncogene vav | 7194 |
| P53621_C453 | COPA Coatomer subunit alpha | 7195 |
| Q96RU3_C248 | FNBP1 Formin-binding protein 1 | 7196 |
| Q13148_C39 | TARDBP TAR DNA-binding protein | 7197 |
| | 43 | |
| Q9NYL9_C132 | TMOD3 Tropomodulin-3 | 7198 |
| Q5VWZ2_C12 | LYPLAL1 Lysophospholipase-like | 7199 |
| | protein 1 | |
| Q7Z5K2_C1170 | WAPAL Wings apart-like protein | 7200 |
| | homolog | |
| Q13148_C244 | TARDBP TAR DNA-binding protein | 7201 |
| | 43 | |
| Q13200_C779 | PSMD2 26S proteasome non-ATPase | 7202 |
| | regulatory subunit 2 | |
| Q99460_C806 | PSMD1 26S proteasome non-ATPase | 7203 |
| | regulatory subunit 1 | |
| P29466_C397 | CASP1 Caspase-1 | 7204 |
| P63279_C138 | UBE2I SUMO-conjugating enzyme | 7205 |
| | UBC9 | |
| Q92608_C1408 | DOCK2 Dedicator of cytokinesis | 7206 |
| | protein 2 | |
| Q9H6A0_C104 | DENND2D DENN domain-containing | 7207 |
| | protein 2D | |
| Q96F86_C410 | EDC3 Enhancer of mRNA-decapping | 7208 |
| | protein 3 | |
| Q12824_C147 | SMARCB1 SWI/SNF-related matrix- | 7209 |
| | associated actin-dependent | |
| O75427_C134 | LRCH4 Leucine-rich repeat and | 7210 |
| | calponin homology domain-containing | |
| | protein 4 | |
| Q08211_C438 | DHX9 ATP-dependent RNA helicase A | 7211 |
| P11387_C630 | TOP1 DNA topoisomerase 1 | 7212 |
| A6NHR9_C985 | SMCHD1 Structural maintenance of | 7213 |
| | chromosomes flexible hinge domain | |
| | containing 1 | |
| P62195_C112 | PSMC5 26S protease regulatory | 7214 |
| | subunit 8 | |
| O43772_C58 | SLC25A20 Mitochondrial | 7215 |
| | carnitine/acylcarnitine carrier protein | |
| Q71U36_C20 | TUBA1A Tubulin alpha-1A chain | 7216 |
| Q15149_C3008 | PLEC Plectin | 7217 |
| O75427_C224 | LRCH4 Leucine-rich repeat and | 7218 |
| | calponin homology domain-containing | |
| | protein 4 | |
| Q8N201_C1633 | INTS1 Integrator complex subunit 1 | 7219 |
| Q71U36_C25 | TUBA1A Tubulin alpha-1A chain | 7220 |
| P07195_C164 | LDHB L-lactate dehydrogenase B | 7221 |
| | chain | |
| P00338_C163 | LDHA L-lactate dehydrogenase A | 7222 |
| | chain | |
| P21333_C1912 | FLNA Filamin-A | 7223 |
| P21333_C1920 | FLNA Filamin-A | 7224 |
| P00558_C379 | PGK1 Phosphoglycerate kinase 1 | 7225 |
| P19447_C342 | ERCC3 TFIIH basal transcription | 7226 |
| | factor complex helicase | |
| P10809_C447 | HSPD1 60 kDa heat shock protein, | 7227 |
| | mitochondrial | |
| P54198_C882 | HIRA Protein HIRA | 7228 |
| P15153_C157 | RAC2 Ras-related C3 botulinum toxin | 7229 |
| | substrate 2 | |
| P53618_C623 | COPB1 Coatomer subunit beta | 7230 |
| P07864_C163 | LDHC L-lactate dehydrogenase C | 7231 |
| | chain | |
| Q6ZMR3_C163 | LDHAL6A L-lactate dehydrogenase A- | 7232 |
| | like 6A | |
| P12268_C140 | IMPDH2 Inosine-5-monophosphate | 7233 |
| | dehydrogenase 2 | |
| Q69YN4_C353 | KIAA1429 Protein virilizer homolog | 7234 |
| P00558_C380 | PGK1 Phosphoglycerate kinase 1 | 7235 |
| P15121_C299 | AKR1B1 Aldose reductase | 7236 |
| Q92888_C892 | ARHGEF1 Rho guanine nucleotide | 7237 |
| | exchange factor 1 | |
| Q9BVQ7_C309 | SPATA5L1 Spermatogenesis- | 7238 |
| | associated protein 5-like protein | |
| Q15149_C3110 | PLEC Plectin | 7239 |
| P17655_C82 | CAPN2 Calpain-2 catalytic subunit | 7240 |
| P50416_C304 | CPT1A Carnitine O- | 7241 |
| | palmitoyltransferase 1, liver isoform | |
| Q92947_C289 | GCDH Glutaryl-CoA dehydrogenase, | 7242 |
| | mitochondrial | |
| P60763_C157 | RAC3 Ras-related C3 botulinum toxin | 7243 |
| | substrate 3 | |
| P63000_C157 | RAC1 Ras-related C3 botulinum toxin | 7244 |
| | substrate 1 | |
| Q14141_C269 | SEPT6 Septin-6 | 7245 |
| Q6IBS0_C141 | TWF2 Twinfilin-2 | 7246 |
| P46736_C273 | BRCC3 Lys-63-specific deubiquitinase | 7247 |
| | BRCC36 | |
| O14641_C354 | DVL2 Segment polarity protein | 7248 |
| | dishevelled homolog DVL-2 | |
| Q13423_C936 | NNT NAD(P) transhydrogenase, | 7249 |
| | mitochondrial | |
| Q15027_C320 | ACAP1 Arf-GAP with coiled-coil, | 7250 |
| | ANK repeat and PH domain 1 | |
| P22059_C224 | OSBP Oxysterol-binding protein 1 | 7251 |
| Q08945_C139 | SSRP1 FACT complex subunit SSRP1 | 7252 |
| P00558_C367 | PGK1 Phosphoglycerate kinase 1 | 7253 |
| P68366_C305 | TUBA4A Tubulin alpha-4A chain | 7254 |
| Q71U36_C305 | TUBA1A Tubulin alpha-1A chain | 7255 |
| P21333_C574 | FLNA Filamin-A | 7256 |
| Q86XP3_C281 | DDX42 ATP-dependent RNA helicase | 7257 |
| | DDX42 | |
| Q9UL46_C91 | PSME2 Proteasome activator complex | 7258 |
| | subunit 2 | |
| Q92598_C34 | HSPH1 Heat shock protein 105 kDa | 7259 |
| Q16186_C88 | ADRM1 Proteasomal ubiquitin | 7260 |
| | receptor ADRM1 | |
| P21333_C478 | FLNA Filamin-A | 7261 |
| P21333_C483 | FLNA Filamin-A | 7262 |
| P40926_C212 | MDH2 Malate dehydrogenase, | 7263 |
| | mitochondrial | |
| P47756_C36 | CAPZB F-actin-capping protein | 7264 |
| | subunit beta | |
| P12270_C1068 | TPR Nucleoprotein TPR | 7265 |
| O43707_C499 | ACTN4 Alpha-actinin-4 | 7266 |
| Q5VSL9_C798 | FAM40A Protein FAM40A | 7267 |
| P19367_C834 | HK1 Hexokinase-1 | 7268 |
| Q8N9T8_C673 | KRI1 Protein KRI1 homolog | 7269 |
| P68371_C239 | TUBB4B Tubulin beta-4B chain | 7270 |
| Q9BVA1_C239 | TUBB2B Tubulin beta-2B chain | 7271 |
| P07437_C239 | TUBB Tubulin beta chain | 7272 |
| P35579_C896 | MYH9 Myosin-9 | 7273 |
| Q9Y2H1_C235 | STK38L Serine/threonine-protein | 7274 |
| | kinase 38-like | |
| Q15208_C234 | STK38 Serine/threonine-protein kinase | 7275 |
| | 38 | |
| Q9UNM6_C357 | PSMD13 26S proteasome non-ATPase | 7276 |
| | regulatory subunit 13 | |
| Q8N8A2_C921 | ANKRD44 Serine/threonine-protein | 7277 |
| | phosphatase 6 regulatory | |
| Q14669_C1959 | TRIP12 E3 ubiquitin-protein ligase | 7278 |
| | TRIP12 | |
| Q8IV53_C21 | DENND1C DENN domain-containing | 7279 |
| | protein 1C | |
| Q86X10_C327 | RALGAPB Ral GTPase-activating | 7280 |
| | protein subunit beta | |
| P0CB43_C368 | FAM203B Protein FAM203B | 7281 |
| Q9Y490_C1434 | TLN1 Talin-1 | 7282 |
| O00499_C47 | BIN1 Myc box-dependent-interacting | 7283 |
| | protein 1 | |
| O15067_C66 | PFAS | 7284 |
| | Phosphoribosylfonnylglycinamidine | |
| | synthase | |
| Q01082_C112 | SPTBN1 Spectrin beta chain, non- | 7285 |
| | erythrocytic 1 | |
| Q92793_C1219 | CREBBP CREB-binding protein | 7286 |
| Q09472_C1183 | EP300 Histone acetyltransferase p300 | 7287 |
| Q16181_C126 | SEPT7 Septin-7 | 7288 |
| Q14289_C972 | PTK2B Protein-tyrosine kinase 2-beta | 7289 |
| Q9H3P2_C131 | WHSC2 Negative elongation factor A | 7290 |
| Q9H3P2_C141 | WHSC2 Negative elongation factor A | 7291 |
| Q92570_C397 | NR4A3 Nuclear receptor subfamily 4 | 7292 |
| | group A member 3 | |
| Q14204_C633 | DYNC1H1 Cytoplasmic dynein 1 | 7293 |
| | heavy chain 1 | |
| P15880_C229 | RPS2 40S ribosomal protein S2 | 7294 |
| Q12906_C278 | ILF3 Interleukin enhancer-binding | 7295 |
| | factor 3 | |
| Q12906_C295 | ILF3 Interleukin enhancer-binding | 7296 |
| | factor 3 | |
| P27348_C237 | YWHAQ 14-3-3 protein theta | 7297 |
| Q9UHD8_C531 | SEPT9 Septin-9 | 7298 |
| P16152_C227 | CBR1 Carbonyl reductase | 7299 |
| P05455_C245 | SSB Lupus La protein | 7300 |
| P50995_C294 | ANXA11 Annexin A11 | 7301 |
| P42765_C382 | ACAA2 3-ketoacyl-CoA thiolase, | 7302 |
| | mitochondrial | |
| Q06323_C101 | PSME1 Proteasome activator complex | 7303 |
| | subunit 1 | |
| Q06323_C106 | PSME1 Proteasome activator complex | 7304 |
| | subunit 1 | |
| P68036_C86 | UBE2L3 Ubiquitin-conjugating | 7305 |
| | enzyme E2 L3 | |
| Q9P0J1_C151 | PDP1 | 7306 |
| Q92973_C285 | TNPO1 Transportin-1 | 7307 |
| Q86UX7_C128 | FERMT3 Fermitin family homolog 3 | 7308 |
| Q9Y5T5_C205 | USP16 Ubiquitin carboxyl-terminal | 7309 |
| | hydrolase 16 | |
| Q15149_C4071 | PLEC Plectin | 7310 |
| Q9NP80_C714 | PNPLA8 Calcium-independent | 7311 |
| | phospholipase A2-gamma | |
| P13796_C140 | LCP1 Plastin-2 | 7312 |
| O14647_C365 | CHD2 Chromodomain-helicase-DNA- | 7313 |
| | binding protein 2 | |
| Q92973_C297 | TNPO1 Transportin-1 | 7314 |
| P13489_C95 | RNH1 Ribonuclease inhibitor | 7315 |
| P13489_C96 | RNH1 Ribonuclease inhibitor | 7316 |
| Q9HC35_C820 | EML4 Echinoderm microtubule- | 7317 |
| | associated protein-like 4 | |
| P08107_C603 | HSPA1B Heat shock 70 kDa protein | 7318 |
| | 1A/1B | |
| P30510_C345 | HLA-C HLA class I histocompatibility | 7319 |
| | antigen, Cw-14 alph | |
| Q29865_C345 | HLA-C HLA class I histocompatibility | 7320 |
| | antigen, Cw-18 alph | |
| P30504_C345 | HLA-C HLA class I histocompatibility | 7321 |
| | antigen, Cw-4 alpha | |
| Q00653_C432 | NFKB2 Nuclear factor NF-kappa-B | 7322 |
| | p100 subunit | |
| Q96GX9_C16 | APIP Probable methylthioribulose-1- | 7323 |
| | phosphate dehydratase | |
| P16152_C226 | CBR1 Carbonyl reductase | 7324 |
| P13797_C143 | PLS3 Plastin-3 | 7325 |
| Q9TNN7_C345 | HLA-C HLA class I histocompatibility | 7326 |
| | antigen, Cw-5 alpha | |
| Q29963_C345 | HLA-C HLA class I histocompatibility | 7327 |
| | antigen, Cw-6 alpha | |
| Q29960_C345 | HLA-C HLA class I histocompatibility | 7328 |
| | antigen, Cw-16 alpha | |
| Q07000_C345 | HLA-C HLA class I histocompatibility | 7329 |
| | antigen, Cw-15 alpha | |
| P14678_C19 | SNRPB Small nuclear | 7330 |
| | ribonucleoprotein-associated protein | |
| P63162_C19 | SNRPN Small nuclear | 7331 |
| | ribonucleoprotein-associated protein | |
| O14950_C109 | MYL12B Myosin regulatory light chain | 7332 |
| | 12B | |
| P14618_C358 | PKM Pyruvate kinase isozymes M1/M2 | 7333 |
| O14733_C131 | MAP2K7 Dual specificity mitogen- | 7334 |
| | activated protein kinase | |
| P62701_C41 | RPS4X 40S ribosomal protein S4, X | 7335 |
| | isoform | |
| P60709_C257 | ACTB Actin, cytoplasmic 1 | 7336 |
| P60709_C272 | ACTB Actin, cytoplasmic 1 | 7337 |
| Q8N7H5_C36 | PAF1 RNA polymerase II-associated | 7338 |
| | factor 1 homolog | |
| Q9UBB5_C359 | MBD2 Methyl-CpG-binding domain | 7339 |
| | protein 2 | |
| P21964_C223 | COMT Catechol O-methyltransferase | 7340 |
| Q9BVQ7_C509 | SPATA5L1 Spermatogenesis- | 7341 |
| | associated protein 5-like protein | |
| P61247_C201 | RPS3A 40S ribosomal protein S3a | 7342 |
| Q9Y3D5_C90 | MRPS18C 28S ribosomal protein S18c, | 7343 |
| | mitochondrial | |
| P48735_C308 | IDH2 Isocitrate dehydrogenase | 7344 |
| P84243_C111 | H3F3B Histone H3.3 | 7345 |
| P37198_C475 | NUP62 Nuclear pore glycoprotein p62 | 7346 |
| P61158_C307 | ACTR3 Actin-related protein 3 | 7347 |
| P35754_C23 | GLRX Glutaredoxin-1 | 7348 |
| Q9Y5B9_C574 | SUPT16H FACT complex subunit | 7349 |
| | SPT16 | |
| P22102_C237 | GART Trifunctional purine | 7350 |
| | biosynthetic protein adenosine | |
| Q01780_C612 | EXOSC10 Exosome component 10 | 7351 |
| Q7Z5K2_C293 | WAPAL Wings apart-like protein | 7352 |
| | homolog | |
| P39687_C87 | ANP32A Acidic leucine-rich nuclear | 7353 |
| | phosphoprotein 32 family member A | |
| P49588_C947 | AARS Alanine--tRNA ligase, | 7354 |
| | cytoplasmic | |
| Q9GZR2_C382 | REXO4 RNA exonuclease 4 | 7355 |
| H3BQZ7_C408 | Uncharacterized protein | 7356 |
| Q1KMD3_C408 | HNRNPUL2 Heterogeneous nuclear | 7357 |
| | ribonucleoprotein U-like protein 2 | |
| Q00325_C237 | SLC25A3 Phosphate carrier protein, | 7358 |
| | mitochondrial | |
| Q8TEM1_C767 | NUP210 Nuclear pore membrane | 7359 |
| | glycoprotein 210 | |
| P62753_C12 | RPS6 40S ribosomal protein S6 | 7360 |
| O95487_C562 | SEC24B Protein transport protein | 7361 |
| | Sec24B | |
| P17844_C200 | DDX5 Probable ATP-dependent RNA | 7362 |
| | helicase DDX5 | |
| Q92841_C277 | DDX17 Probable ATP-dependent RNA | 7363 |
| | helicase DDX17 | |
| O75533_C1035 | SF3B1 Splicing factor 3B subunit 1 | 7364 |
| P62280_C131 | RPS11 40S ribosomal protein S11 | 7365 |
| Q9Y3L3_C102 | SH3BP1 SH3 domain-binding protein 1 | 7366 |
| Q13148_C50 | TARDBP TAR DNA-binding protein | 7367 |
| | 43 | |
| P14649_C89 | MYL6B Myosin light chain 6B | 7368 |
| P60660_C32 | MYL6 Myosin light polypeptide 6 | 7369 |
| P62910_C91 | RPL32 60S ribosomal protein L32 | 7370 |
| P21333_C1157 | FLNA Filamin-A | 7371 |
| Q9BWD1_C88 | ACAT2 Acetyl-CoA acetyltransferase, | 7372 |
| | cytosolic | |
| Q9BWD1_C92 | ACAT2 Acetyl-CoA acetyltransferase, | 7373 |
| | cytosolic | |
| Q8WWP7_C181 | GIMAP1 GTPase IMAP family | 7374 |
| | member 1 | |
| P05386_C61 | RPLP1 60S acidic ribosomal protein P1 | 7375 |
| P07900_C374 | HSP90AA1 Heat shock protein HSP | 7376 |
| | 90-alpha | |
| O75694_C974 | NUP155 Nuclear pore complex protein | 7377 |
| | Nup155 | |
| Q9NZ32_C388 | ACTR10 Actin-related protein 10 | 7378 |
| P14618_C326 | PKM Pyruvate kinase isozymes M1/M2 | 7379 |
| Q9BZL6_C217 | PRKD2 Serine/threonine-protein kinase | 7380 |
| | D2 | |
| Q9Y490_C709 | TLN1 Talin-1 | 7381 |
| Q9Y490_C719 | TLN1 Talin-1 | 7382 |
| A6NHR9_C505 | SMCHD1 Structural maintenance of | 7383 |
| | chromosomes flexible hinge domain- | |
| | containing protein 1 | |
| P18669_C153 | PGAM1 Phosphoglycerate mutase 1 | 7384 |
| Q99973_C149 | TEP1 Telomerase protein component 1 | 7385 |
| P31150_C202 | GDI1 Rab GDP dissociation inhibitor | 7386 |
| | alpha | |
| Q96S15_C474 | WDR24 WD repeat-containing protein | 7387 |
| | 24 | |
| A0FGR8_C611 | ESYT2 Extended synaptotagmin-2 | 7388 |
| P49792_C348 | RANBP2 E3 SUMO-protein ligase | 7389 |
| | RanBP2 | |
| Q9Y6C9_C79 | MTCH2 Mitochondrial carrier homolog | 7390 |
| | 2 | |
| Q9Y3L3_C388 | SH3BP1 SH3 domain-binding protein 1 | 7391 |
| P47755_C111 | CAPZA2 F-actin-capping protein | 7392 |
| | subunit alpha-2 | |
| Q86W42_C314 | THOC6 THO complex subunit 6 | 7393 |
| | homolog | |
| P38646_C317 | HSPA9 Stress-70 protein, | 7394 |
| | mitochondrial | |
| Q9U1D3_C190 | VPS51 Vacuolar protein sorting- | 7395 |
| | associated protein 51 homolog | |
| Q9Y2Q3_C176 | GSTK1 Glutathione S-transferase | 7396 |
| | kappa 1 | |
| Q86XR8_C285 | CEP57 Centrosomal protein of 57 kDa | 7397 |
| P21281_C162 | ATP6V1B2 V-type proton ATPase | 7398 |
| | subunit B, brain isoform | |
| Q86UV5_C39 | USP48 Ubiquitin carboxyl-terminal | 7399 |
| | hydrolase 48 | |
| P35579_C671 | MYH9 Myosin-9 | 7400 |
| P10809_C237 | HSPD1 60 kDa heat shock protein, | 7401 |
| | mitochondrial | |
| P35611_C68 | ADD1 Alpha-adducin | 7402 |
| Q9H4M9_C138 | EHD1 EH domain-containing protein 1 | 7403 |
| O60828_C60 | PQBP1 Polyglutamine-binding protein | 7404 |
| | 1 | |
| Q9P289_C392 | MST4 Serine/threonine-protein kinase | 7405 |
| | MST4 | |
| P28838_C376 | LAP3 Cytosol aminopeptidase | 7406 |
| P55265_C851 | ADAR Double-stranded RNA-specific | 7407 |
| | adenosine deaminase | |
| Q8WXH0_C39 | SYNE2 Nesprin-2 | 7408 |
| P50991_C252 | CCT4 T-complex protein 1 subunit | 7409 |
| | delta | |
| Q8NDH3_C189 | NPEPL1 Probable aminopeptidase | 7410 |
| | NPEPL1 | |
| P49023_C290 | PXN Paxillin | 7411 |
| Q9Y508_C8 | RNF114 RING finger protein 114 | 7412 |
| P13667_C555 | PDIA4 Protein disulfide-isomerase A4 | 7413 |
| P30101_C406 | PDIA3 Protein disulfide-isomerase A3 | 7414 |
| P15153_C6 | RAC2 Ras-related C3 botulinum toxin | 7415 |
| | substrate 2 | |
| Q29RF7_C583 | PDS5A Sister chromatid cohesion | 7416 |
| | protein PDS5 homolog A | |
| Q00839_C408 | HNRNPU Heterogeneous nuclear | 7417 |
| | ribonucleoprotein U | |
| P13667_C558 | PDIA4 Protein disulfide-isomerase A4 | 7418 |
| P30101_C409 | PDIA3 Protein disulfide-isomerase A3 | 7419 |
| Q9Y4H4_C160 | GPSM3 G-protein-signaling modulator | 7420 |
| | 3 | |
| P60763_C6 | RAC3 Ras-related C3 botulinum toxin | 7421 |
| | substrate 3 | |
| P63000_C6 | RAC1 Ras-related C3 botulinum toxin | 7422 |
| | substrate 1 | |
| O95232_C40 | LUC7L3 Luc7-like protein 3 | 7423 |
| P60953_C6 | CDC42 Cell division control protein 42 | 7424 |
| | homolog | |
| P61158_C34 | ACTR3 Actin-related protein 3 | 7425 |
| Q9P2A4_C137 | ABI3 ABI gene family member 3 | 7426 |
| P16333_C266 | NCK1 Cytoplasmic protein NCK1 | 7427 |
| P49591_C438 | SARS Serine--tRNA ligase, | 7428 |
| | cytoplasmic | |
| Q9BQC3_C251 | DPH2 Diphthamide biosynthesis | 7429 |
| | protein 2 | |
| Q99439_C61 | CNN2 Calponin-2 | 7430 |
| Q07020_C134 | RPL18 60S ribosomal protein L18 | 7431 |
| P57772_C442 | EEFSEC Selenocysteine-specific | 7432 |
| | elongation factor | |
| Q92797_C848 | SYMPK Symplekin | 7433 |
| Q8WUM4_C250 | PDCD6IP Programmed cell death 6- | 7434 |
| | interacting protein | |
| P04075_C73 | ALDOA Fructose-bisphosphate | 7435 |
| | aldolase A | |
| P31146_C51 | CORO1A Coronin-1A | 7436 |
| P31146_C24 | CORO1A Coronin-1A | 7437 |
| Q9NXE8_C107 | CWC25 Pre-mRNA-splicing factor | 7438 |
| | CWC25 homolog | |
| Q01813_C360 | PFKP 6-phosphofructokinase type C | 7439 |
| Q92878_C1302 | RAD50 DNA repair protein RAD50 | 7440 |
| P62241_C174 | RPS8 40S ribosomal protein S8 | 7441 |
| Q9BQC3_C250 | DPH2 Diphthamide biosynthesis | 7442 |
| | protein 2 | |
| Q14166_C612 | TTLL12 Tubulin--tyrosine ligase-like | 7443 |
| | protein 12 | |
| P62241_C72 | RPS8 40S ribosomal protein S8 | 7444 |
| P28062_C160 | PSMB8 Proteasome subunit beta type-8 | 7445 |
| P51649_C340 | ALDH5A1 Succinate-semialdehyde | 7446 |
| | dehydrogenase, mitochondrial | |
| Q8WYJ6_C99 | SEPT1 Septin-1 | 7447 |
| Q8WYJ6_C102 | SEPT1 Septin-1 | 7448 |
| Q15027_C64 | ACAP1 Arf-GAP with coiled-coil, | 7449 |
| | ANK repeat and PH domain 1 | |
| Q15366_C54 | PCBP2 Poly(rC)-binding protein 2 | 7450 |
| Q15365_C54 | PCBP1 Poly(rC)-binding protein 1 | 7451 |
| P35236_C204 | PTPN7 Tyrosine-protein phosphatase | 7452 |
| | non-receptor type 7 | |
| P47756_C147 | CAPZB F-actin-capping protein | 7453 |
| | subunit beta | |
| A6NHR9_C444 | SMCHD1 Structural maintenance of | 7454 |
| | chromosomes flexible hinge domain- | |
| | containing protein 1 | |
| Q5TDH0_C361 | DDI2 Protein DDI1 homolog 2 | 7455 |
| P62266_C90 | RPS23 40S ribosomal protein S23 | 7456 |
| P29590_C204 | PML Protein PML | 7457 |
| P42226_C228 | STAT6 Signal transducer and activator | 7458 |
| | of transcription 6 | |
| Q9H1E5_C326 | TMX4 Thioredoxin-related | 7459 |
| | transmembrane protein 4 | |
| P62241_C71 | RPS8 40S ribosomal protein S8 | 7460 |
| Q9UBW5_C205 | BIN2 Bridging integrator 2 | 7461 |
| P41214_C489 | EIF2D Eukaryotic translation initiation | 7462 |
| | factor 2D | |
| Q96I99_C255 | SUCLG2 Succinyl-CoA ligase | 7463 |
| P63208_C160 | SKP1 S-phase kinase-associated | 7464 |
| | protein 1 | |
| P61163_C34 | ACTR1A Alpha-centractin | 7465 |
| P42025_C34 | ACTR1B Beta-centractin | 7466 |
| O15381_C626 | NVL Nuclear valosin-containing | 7467 |
| | protein-like | |
| Q9NTX5_C133 | ECHDC1 Ethylmalonyl-CoA | 7468 |
| | decarboxylase | |
| O43390_C292 | HNRNPR Heterogeneous nuclear | 7469 |
| | ribonucleoprotein R | |
| O60506_C289 | SYNCRIP Heterogeneous nuclear | 7470 |
| | ribonucleoprotein Q | |
| Q2M2I8_C156 | AAK1 AP2-associated protein kinase 1 | 7471 |
| P62917_C115 | RPL8 60S ribosomal protein L8 | 7472 |
| O60610_C314 | DIAPH1 Protein diaphanous homolog 1 | 7473 |
| P12081_C455 | HARS Histidine--tRNA ligase, | 7474 |
| | cytoplasmic | |
| P62917_C114 | RPL8 60S ribosomal protein L8 | 7475 |
| P23528_C147 | CFL1 Cofilin-1 | 7476 |
| P49189_C288 | ALDH9A1 4- | 7477 |
| | trimethylaminobutyraldehyde | |
| | dehydrogenase | |
| P49189_C289 | ALDH9A1 4- | 7478 |
| | trimethylaminobutyraldehyde | |
| | dehydrogenase | |
| Q8N0W3_C582 | FUK L-fucose kinase | 7479 |
| Q14137_C404 | BOP1 Ribosome biogenesis protein | 7480 |
| | BOP1 | |
| Q9Y4K1_C1404 | AIM1 Absent in melanoma 1 protein | 7481 |
| P19367_C813 | HK1 Hexokinase-1 | 7482 |
| P60174_C79 | TPI1 Triosephosphate isomerase | 7483 |
| Q9H307_C249 | PNN Pinin | 7484 |
| Q9UKA8_C239 | RCAN3 Calcipressin-3 | 7485 |
| O00160_C101 | MYO1F Unconventional myosin-If | 7486 |
| P07355_C133 | ANXA2 Annexin A2 | 7487 |
| P07814_C1148 | EPRS Bifunctional glutamate/proline-- | 7488 |
| | tRNA ligase | |
| P52907_C157 | CAPZA1 F-actin-capping protein | 7489 |
| | subunit alpha-1 | |
| P62837_C111 | UBE2D2 Ubiquitin-conjugating | 7490 |
| | enzyme E2 D2 | |
| P61077_C111 | UBE2D3 Ubiquitin-conjugating | 7491 |
| | enzyme E2 D3 | |
| Q8NE71_C365 | ABCF1 ATP-binding cassette sub- | 7492 |
| | family F member 1 | |
| Q8NBU5_C359 | ATAD1 ATPase family AAA domain- | 7493 |
| | containing protein 1 | |
| P55265_C630 | ADAR Double-stranded RNA-specific | 7494 |
| | adenosine deaminase | |
| Q9Y3Z3_C51 | SAMHD1 SAM domain and HD | 7495 |
| | domain-containing protein 1 | |
| P29728_C668 | OAS2 2-5-oligoadenylate synthase 2 | 7496 |
| O43823_C85 | AKAP8 A-kinase anchor protein 8 | 7497 |
| P51610_C135 | HCFC1 Host cell factor 1 | 7498 |
| P20592_C100 | MX2 Interferon-induced GTP-binding | 7499 |
| | protein Mx2 | |
| Q96N96_C124 | SPATA13 Spermatogenesis-associated | 7500 |
| | protein 13 | |
| Q01826_C209 | SATB1 DNA-binding protein SATB1 | 7501 |
| I3L1I5_C86 | Uncharacterized protein | 7502 |
| P62837_C107 | UBE2D2 Ubiquitin-conjugating | 7503 |
| | enzyme E2 D2 | |
| P61077_C107 | UBE2D3 Ubiquitin-conjugating | 7504 |
| | enzyme E2 D3 | |
| P20591_C52 | MX1 Interferon-induced GTP-binding | 7505 |
| | protein Mx1 | |
| Q9Y490_C1023 | TLN1 Talin-1 | 7506 |
| Q99973_C1468 | TEP1 Telomerase protein component 1 | 7507 |
| Q8IY67_C297 | RAVER1 Ribonucleoprotein PTB- | 7508 |
| | binding 1 | |
| Q8WUM4_C512 | PDCD6IP Programmed cell death 6- | 7509 |
| | interacting protein | |
| Q14141_C14 | SEPT6 Septin-6 | 7510 |
| O15213_C172 | WDR46 WD repeat-containing protein | 7511 |
| | 46 | |
| B0I1T2_C143 | MYO1G Unconventional myosin-Ig | 7512 |
| P14868_C349 | DARS Aspartate--tRNA ligase, | 7513 |
| | cytoplasmic | |
| P40227_C406 | CCT6A T-complex protein 1 subunit | 7514 |
| | zeta | |
| Q29RF7_C327 | PDS5A Sister chromatid cohesion | 7515 |
| | protein PDS5 homolog A | |
| Q8NF50_C170 | DOCK8 Dedicator of cytokinesis | 7516 |
| | protein 8 | |
| Q14683_C1115 | SMC1A Structural maintenance of | 7517 |
| | chromosomes protein 1A | |
| O14773_C522 | TPP1 Tripeptidyl-peptidase 1 | 7518 |
| O14773_C526 | TPP1 Tripeptidyl-peptidase 1 | 7519 |
| O14773_C537 | TPP1 Tripeptidyl-peptidase 1 | 7520 |
| Q9NTI5_C317 | PDS5B Sister chromatid cohesion | 7521 |
| | protein PDS5 homolog B | |
| Q9NVP1_C183 | DDX18 ATP-dependent RNA helicase | 7522 |
| | DDX18 | |
| Q9Y490_C1506 | TLN1 Talin-1 | 7523 |
| P13796_C164 | LCP1 Plastin-2 | 7524 |
| Q5EBM0_C287 | CMPK2 UMP-CMP kinase 2, | 7525 |
| | mitochondrial | |
| Q08AF3_C875 | SLFN5 Schlafen family member 5 | 7526 |
| Q9NUV9_C220 | GIMAP4 GTPase IMAP family | 7527 |
| | member 4 | |
| P08238_C589 | HSP90AB1 Heat shock protein HSP | 7528 |
| | 90-beta | |
| P08238_C590 | HSP90AB1 Heat shock protein HSP | 7529 |
| | 90-beta | |
| P60866_C70 | RPS20 40S ribosomal protein S20 | 7530 |
| Q08J23_C673 | NSUN2 tRNA (cytosine(34)-C(5))- | 7531 |
| | methyltransferase | |
| Q9H4E7_C253 | DEF6 Differentially expressed in FDCP | 7532 |
| | 6 homolog | |
| Q15365_C194 | PCBP 1 Poly(rC)-binding protein 1 | 7533 |
| P30041_C47 | PRDX6 Peroxiredoxin-6 | 7534 |
| P78371_C395 | CCT2 T-complex protein 1 subunit beta | 7535 |
| P46782_C155 | RPS5 40S ribosomal protein S5 | 7536 |
| Q96GM5_C492 | SMARCD1 SWI/SNF-related matrix- | 7537 |
| | associated actin-dependent | |
| P10599_C73 | TXN Thioredoxin | 7538 |
| Q14258_C70 | TRIM25 E3 ubiquitin/ISG15 ligase | 7539 |
| | TRIM25 | |
| Q96RN5_C618 | MED15 Mediator of RNA polymerase | 7540 |
| | II transcription subuni | |
| P45973_C133 | CBX5 Chromobox protein homolog 5 | 7541 |
| Q9UHD8_C248 | SEPT9 Septin-9 | 7542 |
| P39023_C336 | RPL3 60S ribosomal protein L3 | 7543 |
| O14980_C164 | XPO1 Exportin-1 | 7544 |
| Q08J23_C678 | NSUN2 tRNA (cytosine(34)-C(5))- | 7545 |
| | methyltransferase | |
| Q00839_C562 | HNRNPU Heterogeneous nuclear | 7546 |
| | ribonucleoprotein U | |
| Q9BVA1_C127 | TUBB2B Tubulin beta-2B chain | 7547 |
| Q9BVA1_C129 | TUBB2B Tubulin beta-2B chain | 7548 |
| P10599_C32 | TXN Thioredoxin | 7549 |
| Q14289_C677 | PTK2B Protein-tyrosine kinase 2-beta | 7550 |
| Q9H4E7_C244 | DEF6 Differentially expressed in FDCP | 7551 |
| | 6 homolog | |
| P15311_C117 | EZR Ezrin | 7552 |
| P55060_C387 | CSE1L Exportin-2 | 7553 |
| Q13838_C165 | DDX39B Spliceosome RNA helicase | 7554 |
| | DDX39B | |
| A6NE09_C163 | RPSAP58 Protein RPSAP58 | 7555 |
| Q9BTZ2_C209 | DHRS4 Dehydrogenase/reductase SDR | 7556 |
| | family member 4 | |
| Q8IY21_C517 | DDX60 Probable ATP-dependent RNA | 7557 |
| | helicase DDX60 | |
| P04075_C339 | ALDOA Fructose-bisphosphate | 7558 |
| | aldolase A | |
| P13639_C466 | EEF2 Elongation factor 2 | 7559 |
| Q99832_C29 | CCT7 T-complex protein 1 subunit eta | 7560 |
| P12814_C332 | ACTN1 Alpha-actinin-1 | 7561 |
| Q92619_C278 | HMHA1 Minor histocompatibility | 7562 |
| | protein HA-1 | |
| Q13131_C185 | PRKAA1 5-AMP-activated protein | 7563 |
| | kinase catalytic subunit | |
| P54646_C174 | PRKAA2 5-AMP-activated protein | 7564 |
| | kinase catalytic subunit | |
| Q00610_C926 | CLTC Clathrin heavy chain 1 | 7565 |
| Q00610_C934 | CLTC Clathrin heavy chain 1 | 7566 |
| Q99439_C175 | CNN2 Calponin-2 | 7567 |
| B0I1T2_C965 | MYO1G Unconventional myosin-Ig | 7568 |
| Q02252_C317 | ALDH6A1 Methylmalonate- | 7569 |
| | semialdehyde dehydrogenase | |
| P12955_C467 | PEPD Xaa-Pro dipeptidase | 7570 |
| P08238_C366 | HSP90AB1 Heat shock protein HSP | 7571 |
| | 90-beta | |
| P13639_C369 | EEF2 Elongation factor 2 | 7572 |
| P41240_C31 | CSK Tyrosine-protein kinase CSK | 7573 |
| P13639_C812 | EEF2 Elongation factor 2 | 7574 |
| P22736_C551 | NR4A1 Nuclear receptor subfamily 4 | 7575 |
| | group A member 1 | |
| Q8WVV9_C84 | HNRPLL Heterogeneous nuclear | 7576 |
| | ribonucleoprotein L-like | |
| O43707_C351 | ACTN4 Alpha-actinin-4 | 7577 |
| Q9H4B7_C340 | TUBB1 Tubulin beta-1 chain | 7578 |
| O15067_C270 | PFAS | 7579 |
| | Phosphoribosylformylglycinamidine | |
| | synthase | |
| Q13045_C232 | FLII Protein flightless-1 homolog | 7580 |
| Q13057_C144 | COASY Bifunctional coenzyme A | 7581 |
| | synthase | |
| P43243_C230 | MATR3 Matrin-3 | 7582 |
| A5YKK6_C624 | CNOT1 CCR4-NOT transcription | 7583 |
| | complex subunit 1 | |
| Q9Y6D5_C76 | ARFGEF2 Brefeldin A-inhibited | 7584 |
| | guanine nucleotide-exchange | |
| P49915_C489 | GMPS GMP synthase | 7585 |
| Q96BN8_C347 | FAM105B Protein FAM105B | 7586 |
| P43403_C78 | ZAP70 Tyrosine-protein kinase ZAP- | 7587 |
| | 70 | |
| Q9Y3D0_C158 | FAM96B Mitotic spindle-associated | 7588 |
| | MMXD complex subunit MI | |
| Q03518_C795 | TAP1 Antigen peptide transporter 1 | 7589 |
| Q86WS5_C64 | TMPRSS12 Transmembrane protease | 7590 |
| | serine 12 | |
| P50579_C298 | METAP2 Methionine aminopeptidase 2 | 7591 |
| Q14019_C52 | COTL1 Coactosin-like protein | 7592 |
| P34897_C119 | SHMT2 Serine | 7593 |
| | hydroxymethyltransferase, | |
| | mitochondrial | |
| Q86YV9_C695 | HPS6 Hermansky-Pudlak syndrome 6 | 7594 |
| | protein | |
| Q96FX7_C209 | TRMT61A tRNA (adenine(58)-N(1))- | 7595 |
| | methyltransferase catalytic | |
| P23528_C139 | CFL1 Cofilin-1 | 7596 |
| Q96F15_C238 | GIMAP5 GTPase IMAP family | 7597 |
| | member 5 | |
| P62888_C92 | RPL30 60S ribosomal protein L30 | 7598 |
| Q8N163_C644 | KIAA1967 DBIRD complex subunit | 7599 |
| | KIAA1967 | |
| Q9C0K0_C642 | BCL11B B-cell lymphoma/leukemia | 7600 |
| | 11B | |
| P53618_C888 | COPB1 Coatomer subunit beta | 7601 |
| P13639_C751 | EEF2 Elongation factor 2 | 7602 |
| P60763_C178 | RAC3 Ras-related C3 botulinum toxin | 7603 |
| | substrate 3 | |
| P63000_C178 | RAC1 Ras-related C3 botulinum toxin | 7604 |
| | substrate 1 | |
| P38606_C254 | ATP6V1A V-type proton ATPase | 7605 |
| | catalytic subunit A | |
| Q14683_C987 | SMC1A Structural maintenance of | 7606 |
| | chromosomes protein 1A | |
| P23528_C39 | CFL1 Cofilin-1 | 7607 |
| P17987_C147 | TCP1 T-complex protein 1 subunit | 7608 |
| | alpha | |
| P31946_C96 | YWHAB 14-3-3 protein beta/alpha | 7609 |
| Q63HN8_C1748 | RNF213 E3 ubiquitin-protein ligase | 7610 |
| | RNF213 | |
| P46782_C172 | RPS5 40S ribosomal protein S5 | 7611 |
| O00483_C44 | NDUFA4 NADH dehydrogenase | 7612 |
| P50570_C27 | DNM2 Dynamin-2 | 7613 |
| O75427_C179 | LRCH4 Leucine-rich repeat and | 7614 |
| | calponin homology domain-containing | |
| | protein 4 | |
| P22234_C81 | PAICS Multifunctional protein ADE2 | 7615 |
| P34932_C34 | HSPA4 Heat shock 70 kDa protein 4 | 7616 |
| H3BQZ7_C452 | Uncharacterized protein | 7617 |
| Q1KMD3_C452 | HNKNPUL2 Heterogeneous nuclear | 7618 |
| | ribonucleoprotein U-like protein 2 | |
| P62837_C85 | UBE2D2 Ubiquitin-conjugating | 7619 |
| | enzyme E2 D2 | |
| P61077_C85 | UBE2D3 Ubiquitin-conjugating | 7620 |
| | enzyme E2 D3 | |
| P52565_C79 | ARHGDIA Rho GDP-dissociation | 7621 |
| | inhibitor 1 | |
| Q9HCD5_C137 | NCOA5 Nuclear receptor coactivator 5 | 7622 |
| O14733_C260 | MAP2K7 Dual specificity mitogen- | 7623 |
| | activated protein kinase | |
| Q14203_C791 | DCTN1 Dynactin subunit 1 | 7624 |
| Q96JM7_C682 | L3MBTL3 Lethal(3)malignant brain | 7625 |
| | tumor-like protein 3 | |
| O14773_C365 | TPP1 Tripeptidyl-peptidase 1 | 7626 |
| P61158_C408 | ACTR3 Actin-related protein 3 | 7627 |
| P45974_C532 | USP5 Ubiquitin carboxyl-terminal | 7628 |
| | hydrolase 5 | |
| P23368_C120 | ME2 NAD-dependent malic enzyme, | 7629 |
| | mitochondrial | |
| P60903_C62 | S100A10 Protein S100-A10 | 7630 |
| Q07955_C148 | SRSF1 Serine/arginine-rich splicing | 7631 |
| | factor 1 | |
| P10644_C18 | PRKAR1A cAMP-dependent protein | 7632 |
| | kinase type I-alpha regulatory subunit | |
| P07814_C692 | EPRS Bifunctional glutamate/proline-- | 7633 |
| | tRNA ligase | |
| O15355_C13 | PPM1G Protein phosphatase 1G | 7634 |
| P08133_C669 | ANXA6 Annexin A6 | 7635 |
| P22314_C1039 | UBA1 Ubiquitin-like modifier- | 7636 |
| | activating enzyme 1 | |
| Q00839_C594 | HNRNPU Heterogeneous nuclear | 7637 |
| | ribonucleoprotein U | |
| O14980_C34 | XPO1 Exportin-1 | 7638 |
| O43290_C645 | SART1 U4/U6.U5 tri-snRNP- | 7639 |
| | associated protein 1 | |
| Q9Y5Y2_C72 | NUBP2 Cytosolic Fe—S cluster | 7640 |
| | assembly factor NUBP2 | |
| Q15365_C201 | PCBP1 Poly(rC)-binding protein 1 | 7641 |
| P30086_C168 | PEBP1 Phosphatidylethanolamine- | 7642 |
| | binding protein 1 | |
| P11142_C17 | HSPA8 Heat shock cognate 71 kDa | 7643 |
| | protein | |
| O60496_C110 | DOK2 Docking protein 2 | 7644 |
| P85037_C254 | FOXK1 Forkhead box protein K1 | 7645 |
| P54577_C250 | YARS Tyrosine--tRNA ligase, | 7646 |
| | cytoplasmic | |
| Q8IX01_C656 | SUGP2 SURP and G-patch domain- | 7647 |
| | containing protein 2 | |
| P22314_C1040 | UBA1 Ubiquitin-like modifier- | 7648 |
| | activating enzyme 1 | |
| Q709C8_C2177 | VPS13C Vacuolar protein sorting- | 7649 |
| | associated protein 13C | |
| Q709C8_C2440 | VPS13C Vacuolar protein sorting- | 7650 |
| | associated protein 13C | |
| P61158_C189 | ACTR3 Actin-related protein 3 | 7651 |
| P0CW22_C35 | RPS17L 40S ribosomal protein S17- | 7652 |
| | like | |
| Q6KC79_C1795 | NIPBL Nipped-B-like protein | 7653 |
| Q15042_C322 | RAB3GAP1 Rab3 GTPase-activating | 7654 |
| | protein catalytic subunit | |
| P60709_C285 | ACTB Actin, cytoplasmic 1 | 7655 |
| P04899_C112 | GNAI2 Guanine nucleotide-binding | 7656 |
| | protein G(i) subunit al | |
| P23396_C97 | RPS3 40S ribosomal protein S3 | 7657 |
| P00558_C316 | PGK1 Phosphoglycerate kinase 1 | 7658 |
| P39687_C123 | ANP32A Acidic leucine-rich nuclear | 7659 |
| | phosphoprotein 32 family member A | |
| P27708_C1889 | CAD CAD protein | 7660 |
| Q15646_C188 | OASL 2-5-oligoadenylate synthase-like | 7661 |
| | protein | |
| Q9ULA0_C413 | DNPEP Aspartyl aminopeptidase | 7662 |
| Q9HAU5_C578 | UPF2 Regulator of nonsense transcripts | 7663 |
| | 2 | |
| P50995_C384 | ANXA11 Annexin A11 | 7664 |
| P63220_C17 | RPS21 40S ribosomal protein S21 | 7665 |
| Q16822_C306 | PCK2 Phosphoenolpyruvate | 7666 |
| | carboxykinase | |
| P14921_C31 | ETS1 Protein C-ets-1 | 7667 |
| Q9BWU1_C349 | CDK19 Cyclin-dependent kinase 19 | 7668 |
| P60174_C164 | TPI1 Triosephosphate isomerase | 7669 |
| P55809_C67 | OXCT1 Succinyl-CoA:3-ketoacid | 7670 |
| | coenzyme A transferase 1, | |
| P54136_C369 | RARS Arginine--tRNA ligase, | 7671 |
| | cytoplasmic | |
| O60784_C415 | TOM1 Target of Myb protein 1 | 7672 |
| Q16630_C159 | CPSF6 Cleavage and polyadenylation | 7673 |
| | specificity factor subunit 6 | |
| Q13370_C410 | PDE3B cGMP-inhibited 3,5-cyclic | 7674 |
| | phosphodiesterase B | |
| P68104_C234 | EEF1A1 Elongation factor 1-alpha 1 | 7675 |
| P62937_C115 | PPIA Peptidyl-prolyl cis-trans | 7676 |
| | isomerase A | |
| O75390_C211 | CS Citrate synthase, mitochondrial | 7677 |
| Q92688_C123 | ANP32B Acidic leucine-rich nuclear | 7678 |
| | phosphoprotein 32 family member A | |
| A6NHR9_C1656 | SMCHD1 Structural maintenance of | 7679 |
| | chromosomes flexible hinge domain- | |
| | containing protein 1 | |
| Q3B7J2_C377 | GFOD2 Glucose-fructose | 7680 |
| | oxidoreductase domain-containing | |
| O15523_C296 | DDX3Y ATP-dependent RNA helicase | 7681 |
| | DDX3Y | |
| O00571_C298 | DDX3X ATP-dependent RNA helicase | 7682 |
| | DDX3X | |
| P61158_C235 | ACTR3 Actin-related protein 3 | 7683 |
| Q53EL6_C275 | PDCD4 Programmed cell death protein | 7684 |
| | 4 | |
| P05388_C27 | RPLP0 60S acidic ribosomal protein P0 | 7685 |
| Q96I99_C162 | SUCLG2 Succinyl-CoA ligase | 7686 |
| P08133_C114 | ANXA6 Annexin A6 | 7687 |
| P09104_C389 | ENO2 Gamma-enolase | 7688 |
| P06733_C389 | ENO1 Alpha-enolase | 7689 |
| P13929_C389 | ENO3 Beta-enolase | 7690 |
| P19838_C261 | NFKB1 Nuclear factor NF-kappa-B | 7691 |
| | p105 subunit | |
| Q96IJ6_C389 | GMPPA Mannose-1-phosphate | 7692 |
| | guanyltransferase alpha | |
| P78527_C3187 | PRKDC DNA-dependent protein | 7693 |
| | kinase catalytic subunit | |
| P04083_C270 | ANXA1 Annexin A1 | 7694 |
| Q86WV6_C309 | TMEM173 Transmembrane protein | 7695 |
| | 173 | |
| P31949_C13 | S100A11 Protein S100-A11 | 7696 |
| Q16181_C280 | SEPT7 Septin-7 | 7697 |
| P53621_C1185 | COPA Coatomer subunit alpha | 7698 |
| O75179_C210 | ANKRD17 Ankyrin repeat domain- | 7699 |
| | containing protein 17 | |
| P62333_C193 | PSMC6 26S protease regulatory | 7700 |
| | subunit 10B | |
| Q9Y5P6_C245 | GMPPB Mannose-1-phosphate | 7701 |
| | guanyltransferase beta | |
| P31948_C461 | STIP1 Stress-induced-phosphoprotein 1 | 7702 |
| Q96P48_C900 | ARAP1 Arf-GAP with Rho-GAP | 7703 |
| | domain, ANK repeat and PH domain 1 | |
| Q52LJ0_C216 | FAM98B Protein FAM98B | 7704 |
| P46779_C13 | RPL28 60S ribosomal protein L28 | 7705 |
| Q8N5W9_C81 | FAM101B Protein FAM101B | 7706 |
| P50914_C54 | RPL14 60S ribosomal protein L14 | 7707 |
| P00338_C293 | LDHA L-lactate dehydrogenase A | 7708 |
| | chain | |
| P62993_C32 | GRB2 Growth factor receptor-bound | 7709 |
| | protein 2 | |
| P85037_C439 | FOXK1 Forkhead box protein K1 | 7710 |
| Q14103_C126 | HNRNPD Heterogeneous nuclear | 7711 |
| | ribonucleoprotein D0 | |
| Q9NVA2_C41 | SEPT11 Septin-11 | 7712 |
| P43403_C346 | ZAP70 Tyrosine-protein kinase ZAP- | 7713 |
| | 70 | |
| Q562E7_C130 | WDR81 WD repeat-containing protein | 7714 |
| | 81 | |
| O60610_C796 | DIAPH1 Protein diaphanous homolog 1 | 7715 |
| P08133_C552 | ANXA6 Annexin A6 | 7716 |
| Q13418_C346 | ILK Integrin-linked protein kinase | 7717 |
| P49915_C523 | GMPS GMP synthase | 7718 |
| Q15459_C244 | SF3A1 Splicing factor 3A subunit 1 | 7719 |
| Q06124_C259 | PTPN11 Tyrosine-protein phosphatase | 7720 |
| | non-receptor type 11 | |
| Q02338_C288 | BDH1 D-beta-hydroxybutyrate | 7721 |
| | dehydrogenase, mitochondrial | |
| P46060_C338 | RANGAP1 Ran GTPase-activating | 7722 |
| | protein 1 | |
| Q14204_C4216 | DYNC1H1 Cytoplasmic dynein 1 | 7723 |
| | heavy chain 1 | |
| P40926_C285 | MDH2 Malate dehydrogenase, | 7724 |
| | mitochondrial | |
| Q9NX63_C112 | CHCHD3 Coiled-coil-helix-coiled-coil- | 7725 |
| | helix domain-containing protein 3 | |
| P62280_C116 | RPS11 40S ribosomal protein S11 | 7726 |
| Q9BZV1_C125 | UBXN6 UBX domain-containing | 7727 |
| | protein 6 | |
| O00299_C178 | CLIC1 Chloride intracellular channel | 7728 |
| | protein 1 | |
| O75592_C1131 | MYCBP2 Probable E3 ubiquitin- | 7729 |
| | protein ligase MYCBP2 | |
| P61601_C185 | NCALD Neurocalcin-delta | 7730 |
| P15498_C794 | VAV1 Proto-oncogene vav | 7731 |
| P12004_C81 | PCNA Proliferating cell nuclear antigen | 7732 |
| P60981_C23 | DSTN Destrin | 7733 |
| P14618_C474 | PKM Pyruvate kinase isozymes M1/M2 | 7734 |
| Q9Y3F4_C340 | STRAP Serine-threonine kinase | 7735 |
| | receptor-associated protein | |
| O75694_C704 | NUP155 Nuclear pore complex protein | 7736 |
| | Nup155 | |
| Q92888_C594 | ARHGEF1 Rho guanine nucleotide | 7737 |
| | exchange factor 1 | |
| O00264_C129 | PGRMC1 Membrane-associated | 7738 |
| | progesterone receptor component | |
| O15173_C159 | PGRMC2 Membrane-associated | 7739 |
| | progesterone receptor component | |
| Q15691_C228 | MAPRE1 Microtubule-associated | 7740 |
| | protein RP/EB family member | |
| Q13325_C137 | IFIT5 Interferon-induced protein with | 7741 |
| | tetratricopeptide | |
| Q9Y678_C296 | COPG1 Coatomer subunit gamma-1 | 7742 |
| Q5XKP0_C60 | QIL1 Protein QIL1 | 7743 |
| O43776_C438 | NARS Asparagine--tRNA ligase, | 7744 |
| | cytoplasmic | |
| O75531_C85 | BANF1 Barrier-to-autointegration | 7745 |
| | factor | |
| Q00577_C272 | PURA Transcriptional activator protein | 7746 |
| | Pur-alpha | |
| P61758_C113 | VBP1 Prefoldin subunit 3 | 7747 |
| P62330_C155 | ARF6 ADP-ribosylation factor 6 | 7748 |
| P09110_C381 | ACAA1 3-ketoacyl-CoA thiolase, | 7749 |
| | peroxisomal | |
| P60842_C134 | EIF4A1 Eukaryotic initiation factor | 7750 |
| | 4A-I | |
| Q8N1G4_C249 | LRRC47 Leucine-rich repeat- | 7751 |
| | containing protein 47 | |
| Q9Y5Z7_C673 | HCFC2 Host cell factor 2 | 7752 |
| P30485_C125 | HLA-B HLA class I histocompatibility | 7753 |
| | antigen, B-47 alpha | |
| P51812_C579 | RPS6KA3 Ribosomal protein S6 kinase | 7754 |
| | alpha-3 | |
| Q15418_C575 | RPS6KA1 Ribosomal protein S6 kinase | 7755 |
| | alpha-1 | |
| Q9P2J5_C554 | LARS Leucine--tRNA ligase, | 7756 |
| | cytoplasmic | |
| P00558_C108 | PGK1 Phosphoglycerate kinase 1 | 7757 |
| O14966_C120 | RAB7L1 Ras-related protein Rab-7L1 | 7758 |
| P13639_C591 | EEF2 Elongation factor 2 | 7759 |
| P07195_C36 | LDHB L-lactate dehydrogenase B | 7760 |
| | chain | |
| Q96EY7_C642 | PTCD3 Pentatricopeptide repeat- | 7761 |
| | containing protein 3, mitochondrial | |
| Q13469_C355 | NFATC2 Nuclear factor of activated T- | 7762 |
| | cells, cytoplasmic 2 | |
| Q96JH7_C219 | VCPIP1 Deubiquitinating protein | 7763 |
| | VCIP135 | |
| Q92619_C324 | HMHA1 Minor histocompatibility | 7764 |
| | protein HA-1 | |
| O00148_C164 | DDX39A ATP-dependent RNA | 7765 |
| | helicase DDX39A | |
| P16455_C150 | MGMT Methylated-DNA--protein- | 7766 |
| | cysteine methyltransferase | |
| P40763_C259 | STAT3 Signal transducer and activator | 7767 |
| | of transcription 3 | |
| P07203_C202 | GPX1 Glutathione peroxidase 1 | 7768 |
| P08575_C787 | PTPRC Receptor-type tyrosine-protein | 7769 |
| | phosphatase C | |
| Q9Y6E0_C394 | STK24 Serine/threonine-protein kinase | 7770 |
| | 24 | |
| B5ME19_C444 | EIF3CL Eukaryotic translation | 7771 |
| | initiation factor 3 subunit | |
| P33240_C150 | CSTF2 Cleavage stimulation factor | 7772 |
| | subunit 2 | |
| P52597_C267 | HNRNPF Heterogeneous nuclear | 7773 |
| | ribonucleoprotein F | |
| Q9H019_C52 | FAM54B Protein FAM54B | 7774 |
| Q9H479_C24 | FN3K Fructosamine-3-kinase | 7775 |
| O75369_C660 | FLNB Filamin-B | 7776 |
| P62241_C100 | RPS8 40S ribosomal protein S8 | 7777 |
| P30453_C188 | HLA-A HLA class I histocompatibility | 7778 |
| | antigen, A-34 alpha | |
| P01892_C188 | HLA-A HLA class I histocompatibility | 7779 |
| | antigen, A-2 alpha | |
| P01891_C188 | HLA-A HLA class I histocompatibility | 7780 |
| | antigen, A-68 alpha | |
| P30479_C188 | HLA-B HLA class I histocompatibility | 7781 |
| | antigen, B-41 alpha | |
| P30460_C188 | HLA-B HLA class I histocompatibility | 7782 |
| | antigen, B-8 alpha | |
| P43250_C474 | GRK6 G protein-coupled receptor | 7783 |
| | kinase 6 | |
| Q96JB2_C65 | COG3 Conserved oligomeric Golgi | 7784 |
| | complex subunit 3 | |
| P22102_C646 | GART Trifunctional purine | 7785 |
| | biosynthetic protein adenosine | |
| Q9BTC0_C1079 | DIDO1 Death-inducer obliterator 1 | 7786 |
| Q5SY16_C648 | NOL9 Polynucleotide 5-hydroxyl- | 7787 |
| | kinase NOL9 | |
| O75367_C297 | H2AFY Core histone macro-H2A.1 | 7788 |
| P10314_C188 | HLA-A HLA class I histocompatibility | 7789 |
| | antigen, A-32 alpha | |
| P30512_C188 | HLA-A HLA class I histocompatibility | 7790 |
| | antigen, A-29 alpha | |
| P30510_C188 | HLA-C HLA class I histocompatibility | 7791 |
| | antigen, Cw-14 alpha | |
| Q9TNN7_C188 | HLA-C HLA class I histocompatibility | 7792 |
| | antigen, Cw-5 alpha | |
| P30459_C188 | HLA-A HLA class I histocompatibility | 7793 |
| | antigen, A-74 alpha | |
| Q29963_C188 | HLA-C HLA class I histocompatibility | 7794 |
| | antigen, Cw-6 alpha | |
| Q29960_C188 | HLA-C HLA class I histocompatibility | 7795 |
| | antigen, Cw-16 alpha | |
| Q07000_C188 | HLA-C HLA class I histocompatibility | 7796 |
| | antigen, Cw-15 alpha | |
| P16189_C188 | HLA-A HLA class I histocompatibility | 7797 |
| | antigen, A-31 alpha | |
| Q29865_C188 | HLA-C HLA class I histocompatibility | 7798 |
| | antigen, Cw-18 alpha | |
| P30504_C188 | HLA-C HLA class I histocompatibility | 7799 |
| | antigen, Cw-4 alpha | |
| O60343_C1277 | TBC1D4 TBC1 domain family member | 7800 |
| | 4 | |
| Q14152_C478 | EIF3A Eukaryotic translation initiation | 7801 |
| | factor 3 subunit | |
| E9PMF8_C721 | PTPRC Receptor-type tyrosine-protein | 7802 |
| | phosphatase C | |
| Q01831_C60 | XPC DNA repair protein | 7803 |
| | complementing XP-C cells | |
| P55265_C773 | ADAR Double-stranded RNA-specific | 7804 |
| | adenosine deaminase | |
| P30153_C377 | PPP2R1A Serine/threonine-protein | 7805 |
| | phosphatase 2A 65 kDa regulatory | |
| | subunit A alpha isoform | |
| A6NE09_C148 | RPSAP58 Protein RPSAP58 | 7806 |
| Q9TNN7_C125 | HLA-C HLA class I histocompatibility | 7807 |
| | antigen, Cw-5 alpha | |
| Q95604_C125 | HLA-C HLA class I histocompatibility | 7808 |
| | antigen, Cw-17 alpha | |
| Q29963_C125 | HLA-C HLA class I histocompatibility | 7809 |
| | antigen, Cw-6 alpha | |
| Q29960_C125 | HLA-C HLA class I histocompatibility | 7810 |
| | antigen, Cw-16 alpha | |
| Q07000_C125 | HLA-C HLA class I histocompatibility | 7811 |
| | antigen, Cw-15 alpha | |
| Q9ULA0_C144 | DNPEP Aspartyl aminopeptidase | 7812 |
| P53396_C20 | ACLY ATP-citrate synthase | 7813 |
| P35251_C607 | RFC1 Replication factor C subunit 1 | 7814 |
| Q8IZ69_C463 | TRMT2A tRNA (uracil-5-)- | 7815 |
| | methyltransferase homolog A | |
| O00567_C52 | NOP56 Nucleolar protein 56 | 7816 |
| Q5JSL3_C160 | DOCK11 Dedicator of cytokinesis | 7817 |
| | protein 11 | |
| P21333_C649 | FLNA Filamin-A | 7818 |
| P21291_C122 | CSRP1 Cysteine and glycine-rich | 7819 |
| | protein 1 | |
| P22314_C278 | UBA1 Ubiquitin-like modifier- | 7820 |
| | activating enzyme 1 | |
| Q9P289_C410 | MST4 Serine/threonine-protein kinase | 7821 |
| | MST4 | |
| P46782_C66 | RPS5 40S ribosomal protein S5 | 7822 |
| Q92616_C1235 | GCN1L1 Translational activator GCN1 | 7823 |
| P27707_C45 | DCK Deoxycytidine kinase | 7824 |
| Q8NF91_C6415 | SYNE1 Nesprin-1 | 7825 |
| Q92538_C685 | GBF1 Golgi-specific brefeldin A- | 7826 |
| | resistance guanine nucleotide exchange | |
| | factor 1 | |
| P13639_C290 | EEF2 Elongation factor 2 | 7827 |
| Q15120_C41 | PDK3 | 7828 |
| O43765_C153 | SGTA Small glutamine-rich | 7829 |
| | tetratricopeptide repeat-containing | |
| | alpha | |
| P63244_C182 | GNB2L1 Guanine nucleotide-binding | 7830 |
| | protein subunit beta-2-like 1, N- | |
| | terminally processed | |
| P61978_C145 | HNRNPK Heterogeneous nuclear | 7831 |
| | ribonucleoprotein K | |
| Q9Y3D0_C93 | FAM96B Mitotic spindle-associated | 7832 |
| | MMXD complex subunit MI | |
| H3BQZ7_C405 | Uncharacterized protein | 7833 |
| Q1KMD3_C405 | HNRNPUL2 Heterogeneous nuclear | 7834 |
| | ribonucleoprotein U-like protein 2 | |
| Q92888_C815 | ARHGEF1 Rho guanine nucleotide | 7835 |
| | exchange factor 1 | |
| P30050_C17 | RPL12 60S ribosomal protein L12 | 7836 |
| P27348_C134 | YWHAQ 14-3-3 protein theta | 7837 |
| Q9UPW5_C1164 | AGTPBP1 Cytosolic carboxypeptidase | 7838 |
| | 1 | |
| Q13185_C177 | CBX3 Chromobox protein homolog 3 | 7839 |
| P50914_C42 | RPL14 60S ribosomal protein L14 | 7840 |
| P60709_C217 | ACTB Actin, cytoplasmic 1 | 7841 |
| Q6S8J3_C917 | POTEE POTE ankyrin domain family | 7842 |
| | member E | |
| Q52LJ0_C63 | FAM98B Protein FAM98B | 7843 |
| Q8TAQ2_C495 | SMARCC2 SWI/SNF complex subunit | 7844 |
| | SMARCC2 | |
| P04083_C324 | ANXA1 Annexin A1 | 7845 |
| Q86VP6_C413 | CAND1 Cullin-associated NEDD8- | 7846 |
| | dissociated protein 1 | |
| P40925_C137 | MDH1 Malate dehydrogenase, | 7847 |
| | cytoplasmic | |
| P23528_C80 | CFL1 Cofilin-1 | 7848 |
| Q14C86_C568 | GAPVD1 GTPase-activating protein | 7849 |
| | and VPS9 domain-containing protein 1 | |
| P62241_C182 | RPS8 40S ribosomal protein S8 | 7850 |
| Q86WR0_C83 | CCDC25 Coiled-coil domain- | 7851 |
| | containing protein 25 | |
| P00558_C50 | PGK1 Phosphoglycerate kinase 1 | 7852 |
| O00743_C192 | PPP6C Serine/threonine-protein | 7853 |
| | phosphatase 6 catalytic subunit | |
| O75934_C106 | BCAS2 Pre-mRNA-splicing factor | 7854 |
| | SPF27 | |
| Q92616_C1692 | GCN1L1 Translational activator GCN1 | 7855 |
| Q63HN8_C1614 | RNF213 E3 ubiquitin-protein ligase | 7856 |
| | RNF213 | |
| P17655_C405 | CAPN2 Calpain-2 catalytic subunit | 7857 |
| P43487_C132 | RANBP1 Ran-specific GTPase- | 7858 |
| | activating protein | |
| Q9BVP2_C251 | GNL3 Guanine nucleotide-binding | 7859 |
| | protein-like 3 | |
| O75995_C152 | SASH3 SAM and SH3 domain- | 7860 |
| | containing protein 3 | |
| Q7Z6Z7_C4341 | HUWE1 E3 ubiquitin-protein ligase | 7861 |
| | HUWE1 | |
| P21333_C2107 | FLNA Filamin-A | 7862 |
| Q9UQ90_C353 | SPG7 Paraplegin | 7863 |
| H3BN98_C196 | Uncharacterized protein | 7864 |
| P62244_C30 | RPS15A 40S ribosomal protein S15a | 7865 |
| Q9UPN7_C455 | PPP6R1 Serine/threonine-protein | 7866 |
| | phosphatase 6 regulatory | |
| P26641_C266 | EEF1G Elongation factor 1-gamma | 7867 |
| Q96F07_C346 | CYFIP2 Cytoplasmic FMR1- | 7868 |
| | interacting protein 2 | |
| Q16555_C504 | DPYSL2 Dihydropyrimidinase-related | 7869 |
| | protein 2 | |
| Q9H3U1_C426 | UNC45A Protein unc-45 homolog A | 7870 |
| Q9UBT2_C30 | UBA2 SUMO-activating enzyme | 7871 |
| | subunit 2 | |
| O60333_C1661 | KIF1B Kinesin-like protein KIF1B | 7872 |
| P35579_C1379 | MYH9 Myosin-9 | 7873 |
| P17844_C170 | DDX5 Probable ATP-dependent RNA | 7874 |
| | helicase DDX5 | |
| Q92841_C247 | DDX17 Probable ATP-dependent RNA | 7875 |
| | helicase DDX17 | |
| I3L3Q1_C657 | Uncharacterized protein | 7876 |
| Q6ZSZ5_C699 | ARHGEF18 Rho guanine nucleotide | 7877 |
| | exchange factor 18 | |
| Q15233_C145 | NONO Non-POU domain-containing | 7878 |
| | octamer-binding protein | |
| Q9Y490_C1202 | TLN1 Talin-1 | 7879 |
| Q9H0C8_C301 | ILKAP Integrin-linked kinase- | 7880 |
| | associated serine/threonine | |
| Q63HN8_C4111 | RNF213 E3 ubiquitin-protein ligase | 7881 |
| | RNF213 | |
| P54886_C612 | ALDH18A1 Delta-1-pyrroline-5- | 7882 |
| | carboxylate synthase | |
| Q9Y613_C502 | FHOD1 FH1/FH2 domain-containing | 7883 |
| | protein 1 | |
| Q86TI0_C604 | TBC1D1 TBC1 domain family member | 7884 |
| | 1 | |
| O60234_C96 | GMFG Glia maturation factor gamma | 7885 |
| P60983_C96 | GMFB Glia maturation factor beta | 7886 |
| Q9UIS9_C486 | MBD1 Methyl-CpG-binding domain | 7887 |
| | protein 1 | |
| Q9HB19_C332 | PLEKHA2 Pleckstrin homology | 7888 |
| | domain-containing family A member 2 | |
| O60234_C87 | GMFG Glia maturation factor gamma | 7889 |
| P60983_C87 | GMFB Glia maturation factor beta | 7890 |
| Q6FI81_C288 | CIAPIN1 Anamorsin | 7891 |
| Q5T4S7_C1230 | UBR4 E3 ubiquitin-protein ligase | 7892 |
| | UBR4 | |
| Q01826_C173 | SATB1 DNA-binding protein SATB1 | 7893 |
| Q9Y490_C1199 | TLN1 Talin-1 | 7894 |
| Q16656_C229 | NRF1 Nuclear respiratory factor 1 | 7895 |
| P60866_C36 | RPS20 40S ribosomal protein S20 | 7896 |
| Q9H361_C132 | PABPC3 Polyadenylate-binding protein | 7897 |
| | 3 | |
| Q13310_C132 | PABPC4 Polyadenylate-binding protein | 7898 |
| | 4 | |
| P11940_C132 | PABPC1 Polyadenylate-binding protein | 7899 |
| | 1 | |
| P31943_C267 | HNRNPH1 Heterogeneous nuclear | 7900 |
| | ribonucleoprotein H | |
| O43390_C99 | HNRNPR Heterogeneous nuclear | 7901 |
| | ribonucleoprotein R | |
| O60506_C96 | SYNCRIP Heterogeneous nuclear | 7902 |
| | ribonucleoprotein Q | |
| P36578_C208 | RPL4 60S ribosomal protein L4 | 7903 |
| O75170_C247 | PPP6R2 Serine/threonine-protein | 7904 |
| | phosphatase 6 regulatory | |
| Q14644_C58 | RASA3 Ras GTPase-activating protein | 7905 |
| | 3 | |
| Q01433_C230 | AMPD2 AMP deaminase 2 | 7906 |
| P55795_C267 | HNRNPH2 Heterogeneous nuclear | 7907 |
| | ribonucleoprotein H2 | |
| P63104_C94 | YWHAZ 14-3-3 protein zeta/delta | 7908 |
| Q9BXK1_C233 | KLF16 Krueppel-like factor 16 | 7909 |
| Q9UKV8_C188 | EIF2C2 Protein argonaute-2 | 7910 |
| Q9NVM9_C349 | Asun Protein asunder homolog | 7911 |
| P24928_C981 | POLR2A DNA-directed RNA | 7912 |
| | polymerase II subunit RPB1 | |
| Q15283_C314 | RASA2 Ras GTPase-activating protein | 7913 |
| | 2 | |
| P61224_C141 | RAP1B Ras-related protein Rap-1b | 7914 |
| P13804_C155 | ETFA Electron transfer flavoprotein | 7915 |
| | subunit alpha, mitochondrial | |
| Q14353_C91 | GAMT Guanidinoacetate N- | 7916 |
| | methyltransferase | |
| Q92616_C1595 | GCN1L1 Translational activator GCN1 | 7917 |
| O75995_C351 | SASH3 SAM and SH3 domain- | 7918 |
| | containing protein 3 | |
| Q9H270_C890 | VPS11 Vacuolar protein sorting- | 7919 |
| | associated protein 11 homolog | |
| P31948_C26 | STIP1 Stress-induced-phosphoprotein 1 | 7920 |
| Q13303_C248 | KCNAB2 Voltage-gated potassium | 7921 |
| | channel subunit beta-2 | |
| Q86X76_C165 | NIT1 Nitrilase homolog 1 | 7922 |
| P31146_C285 | CORO1A Coronin-1A | 7923 |
| Q02750_C277 | MAP2K1 Dual specificity mitogen- | 7924 |
| | activated protein kinase | |
| O95758_C68 | PTBP3 Polypyrimidine tract-binding | 7925 |
| | protein 3 | |
| P53621_C380 | COPA Coatomer subunit alpha | 7926 |
| Q9BUJ2_C487 | HNRNPUL1 Heterogeneous nuclear | 7927 |
| | ribonucleoprotein U-like protein | |
| Q92619_C469 | HMHA1 Minor histocompatibility | 7928 |
| | protein HA-1 | |
| Q9Y228_C380 | TRAF3IP3 TRAF3-interacting JNK- | 7929 |
| | activating modulator | |
| Q9BW61_C25 | DDA1 DET1- and DDB1-associated | 7930 |
| | protein 1 | |
| P21333_C1018 | FLNA Filamin-A | 7931 |
| O75427_C105 | LRCH4 Leucine-rich repeat and | 7932 |
| | calponin homology domain-containing | |
| | protein 4 | |
| P22314_C481 | UBA1 Ubiquitin-like modifier- | 7933 |
| | activating enzyme 1 | |
| Q8N7H5_C218 | PAF1 RNA polymerase II-associated | 7934 |
| | factor 1 homolog | |
| Q16644_C203 | MAPKAPK3 MAP kinase-activated | 7935 |
| | protein kinase 3 | |
| Q6PD62_C196 | CTR9 RNA polymerase-associated | 7936 |
| | protein CTR9 homolog | |
| P23919_C163 | DTYMK Thymidylate kinase | 7937 |
| Q9UKG1_C462 | APPL1 DCC-interacting protein 13- | 7938 |
| | alpha | |
| Q13155_C306 | AIMP2 Aminoacyl tRNA synthase | 7939 |
| | complex-interacting multif | |
| Q99439_C215 | CNN2 Calponin-2 | 7940 |
| Q99832_C450 | CCT7 T-complex protein 1 subunit eta | 7941 |
| Q8IXI2_C175 | RHOT1 Mitochondrial Rho GTPase 1 | 7942 |
| Q9Y4W2_C474 | LAS1L Ribosomal biogenesis protein | 7943 |
| | LAS1L | |
| P13716_C223 | ALAD Delta-aminolevulinic acid | 7944 |
| | dehydratase | |
| Q8IWX8_C168 | CHERP Calcium homeostasis | 7945 |
| | endoplasmic reticulum protein | |
| P08575_C1167 | PTPRC Receptor-type tyrosine-protein | 7946 |
| | phosphatase C | |
| E9PPU0_C406 | EPPK1 Epiplakin | 7947 |
| P15170_C327 | GSPT1 Eukaryotic peptide chain | 7948 |
| | release factor GTP-binding subunit | |
| | ERF3A | |
| Q9NZN3_C138 | EHD3 EH domain-containing protein 3 | 7949 |
| Q8WUH2_C826 | TGFBRAP1 Transforming growth | 7950 |
| | factor-beta receptor-associate protein 1 | |
| Q9BZZ5_C234 | API5 Apoptosis inhibitor 5 | 7951 |
| P53992_C910 | SEC24C Protein transport protein | 7952 |
| | Sec24C | |
| O15027_C1619 | SEC16A Protein transport protein | 7953 |
| | Sec16A | |
| O43791_C361 | SPOP Speckle-type POZ protein | 7954 |
| O95202_C552 | LETM1 LETM1 and EF-hand domain- | 7955 |
| | containing protein 1, mitochondrial | |
| Q9NZ09_C161 | UBAP1 Ubiquitin-associated protein 1 | 7956 |
| P45880_C103 | VDAC2 Voltage-dependent anion- | 7957 |
| | selective channel protein | |
| Q8IWB7_C401 | WDFY1 WD repeat and FYVE | 7958 |
| | domain-containing protein 1 | |
| O60216_C585 | RAD21 Double-strand-break repair | 7959 |
| | protein rad21 homolog | |
| O00567_C142 | NOP56 Nucleolar protein 56 | 7960 |
| P55265_C649 | ADAR Double-stranded RNA-specific | 7961 |
| | adenosine deaminase | |
| Q8WYP5_C521 | AHCTF1 Protein ELYS | 7962 |
| Q6PKG0_C502 | LARP1 La-related protein 1 | 7963 |
| Q6PJW8_C192 | CNST Consortin | 7964 |
| Q5VSL9_C418 | FAM40A Protein FAM40A | 7965 |
| Q92835_C385 | INPP5D Phosphatidylinositol 3,4,5- | 7966 |
| | trisphosphate 5-phosphatase 1 | |
| Q00610_C736 | CLTC Clathrin heavy chain 1 | 7967 |
| Q14019_C10 | COTL1 Coactosin-like protein | 7968 |
| Q9UEW8_C450 | STK39 STE20/SPS1-related proline- | 7969 |
| | alanine-rich protein kinase | |
| P22626_C50 | HNRNPA2B1 Heterogeneous nuclear | 7970 |
| | ribonucleoproteins A2/B1 | |
| P55795_C22 | HNRNPH2 Heterogeneous nuclear | 7971 |
| | ribonucleoprotein H2 | |
| Q8IXW5_C105 | RPAP2 Putative RNA polymerase II | 7972 |
| | subunit B1 CTD phosphatase RPAP2 | |
| Q92841_C584 | DDX17 Probable ATP-dependent RNA | 7973 |
| | helicase DDX17 | |
| P50552_C334 | VASP Vasodilator-stimulated | 7974 |
| | phosphoprotein | |
| P29728_C523 | OAS2 2-5-oligoadenylate synthase 2 | 7975 |
| Q9BUJ2_C391 | HNRNPUL1 Heterogeneous nuclear | 7976 |
| | ribonucleoprotein U-like protein | |
| Q5VWQ0_C282 | RSBN1 Round spermatid basic protein | 7977 |
| | 1 | |
| O75369_C2556 | FLNB Filamin-B | 7978 |
| P04075_C178 | ALDOA Fructose-bisphosphate | 7979 |
| | aldolase A | |
| Q9UID3_C81 | VPS51 Vacuolar protein sorting- | 7980 |
| | associated protein 51 homolog | |
| Q9ULL5_C173 | PRR12 Proline-rich protein 12 | 7981 |
| P14618_C423 | PKM Pyruvate kinase isozymes M1/M2 | 7982 |
| O15117_C778 | FYB FYN-binding protein | 7983 |
| Q02880_C426 | TOP2B DNA topoisomerase 2-beta | 7984 |
| P14618_C424 | PKM Pyruvate kinase isozymes M1/M2 | 7985 |
| O14976_C913 | GAK Cyclin-G-associated kinase | 7986 |
| Q14980_C1907 | NUMA1 Nuclear mitotic apparatus | 7987 |
| | protein 1 | |
| Q86WI3_C1190 | NLRC5 Protein NLRC5 | 7988 |
| Q6IS14_C129 | EIF5AL1 Eukaryotic translation | 7989 |
| | initiation factor 5A-1-like | |
| Q9Y2X3_C106 | NOP58 Nucleolar protein 58 | 7990 |
| Q9NRG7_C78 | SDR39U1 Epimerase family protein | 7991 |
| | SDR39U1 | |
| Q14761_C134 | PTPRCAP Protein tyrosine | 7992 |
| | phosphatase receptor type C-associated | |
| | protein | |
| P30479_C349 | HLA-B HLA class I histocompatibility | 7993 |
| | antigen, B-41 alpha | |
| P30460_C349 | HLA-B HLA class I histocompatibility | 7994 |
| | antigen, B-8 alpha | |
| Q6KC79_C2151 | NIPBL Nipped-B-like protein | 7995 |
| P26038_C284 | MSN Moesin | 7996 |
| Q9UL15_C118 | BAG5 BAG family molecular | 7997 |
| | chaperone regulator 5 | |
| Q15022_C325 | SUZ12 Polycomb protein SUZ12 | 7998 |
| Q8NBZ0_C179 | INO80E INO80 complex subunit E | 7999 |
| P42226_C355 | STAT6 Signal transducer and activator | 8000 |
| | of transcription 6 | |
| P37802_C63 | TAGLN2 Transgelin-2 | 8001 |
| O60313_C786 | OPA1 Dynamin-like 120 kDa protein, | 8002 |
| | mitochondrial | |
| Q8TB24_C881 | RIN3 Ras and Rab interactor 3 | 8003 |
| P42226_C356 | STAT6 Signal transducer and activator | 8004 |
| | of transcription 6 | |
| P13639_C693 | EEF2 Elongation factor 2 | 8005 |
| P13010_C493 | XRCC5 X-ray repair cross- | 8006 |
| | complementing protein 5 | |
| Q9NX46_C132 | ADPRHL2 Poly(ADP-ribose) | 8007 |
| | glycohydrolase ARH3 | |
| Q5RL73_C199 | RBM48 RNA-binding protein 48 | 8008 |
| P09001_C338 | MRPL3 39S ribosomal protein L3, | 8009 |
| | mitochondrial | |
| Q14204_C3089 | DYNC1H1 Cytoplasmic dynein 1 | 8010 |
| | heavy chain 1 | |
| Q15437_C767 | SEC23B Protein transport protein | 8011 |
| | Sec23B | |
| P30040_C157 | ERP29 Endoplasmic reticulum resident | 8012 |
| | protein 29 | |
| Q9UQ35_C956 | SRRM2 Serine/arginine repetitive | 8013 |
| | matrix protein 2 | |
| Q5RL73_C204 | RBM48 RNA-binding protein 48 | 8014 |
| Q12996_C646 | CSTF3 Cleavage stimulation factor | 8015 |
| | subunit 3 | |
| P15170_C276 | GSPT1 Eukaryotic peptide chain | 8016 |
| | release factor GTP-binding subunit | |
| | ERF3A | |
| Q9BY32_C146 | ITPA Inosine triphosphate | 8017 |
| | pyrophosphatase | |
| Q9Y3Z3_C80 | SAMHD1 SAM domain and HD | 8018 |
| | domain-containing protein 1 | |
| Q99590_C566 | SCAF11 Protein SCAF11 | 8019 |
| Q9BSQ5_C170 | CCM2 Malcavernin | 8020 |
| P07737_C128 | PFN1 Profilin-1 | 8021 |
| P45974_C195 | USP5 Ubiquitin carboxyl-terminal | 8022 |
| | hydrolase 5 | |
| Q9H2U2_C44 | PPA2 Inorganic pyrophosphatase 2, | 8023 |
| | mitochondrial | |
| Q92530_C185 | PSMF1 Proteasome inhibitor PI31 | 8024 |
| | subunit | |
| P26599_C23 | PTBP1 Polypyrimidine tract-binding | 8025 |
| | protein 1 | |
| P57075_C435 | UBASH3A Ubiquitin-associated and | 8026 |
| | SH3 domain-containing protein A | |
| P25208_C89 | NFYB Nuclear transcription factor Y | 8027 |
| | subunit beta | |
| Q8N6S5_C48 | ARL6IP6 ADP-ribosylation factor-like | 8028 |
| | protein 6-interacting protein 6 | |
| Q96AP0_C406 | ACD Adrenocortical dysplasia protein | 8029 |
| | homolog | |
| P25208_C85 | NFYB Nuclear transcription factor Y | 8030 |
| | subunit beta | |
| O14745_C206 | SLC9A3R1 Na(+)/H(+) exchange | 8031 |
| | regulatory cofactor NHE-RF1 | |
| Q9H6S0_C363 | YTHDC2 Probable ATP-dependent | 8032 |
| | RNA helicase YTHDC2 | |
| Q9BY32_C154 | ITPA Inosine triphosphate | 8033 |
| | pyrophosphatase | |
| Q9UQ13_C306 | SHOC2 Leucine-rich repeat protein | 8034 |
| | SHOC-2 | |
| P30876_C177 | POLR2B DNA-directed RNA | 8035 |
| | polymerase II subunit RPB2 | |
| Q8TB24_C484 | RIN3 Ras and Rab interactor 3 | 8036 |
| P37802_C38 | TAGLN2 Transgelin-2 | 8037 |
| P60174_C255 | TPI1 Triosephosphate isomerase | 8038 |
| Q99832_C511 | CCT7 T-complex protein 1 subunit eta | 8039 |
| O75592_C3152 | MYCBP2 Probable E3 ubiquitin- | 8040 |
| | protein ligase MYCBP2 | |
| Q9BSH5_C243 | HDHD3 Haloacid dehalogenase-like | 8041 |
| | hydrolase domain-containing 3 | |
| Q13573_C250 | SNW1 SNW domain-containing | 8042 |
| | protein 1 | |
| P46777_C100 | RPL5 60S ribosomal protein L5 | 8043 |
| O95571_C170 | ETHE1 Protein ETHE1, mitochondrial | 8044 |
| Q92556_C726 | ELMO1 Engulfment and cell motility | 8045 |
| | protein 1 | |
| P84074_C185 | HPCA Neuron-specific calcium- | 8046 |
| | binding protein hippocalcin | |
| P37235_C185 | HPCAL1 Hippocalcin-like | 8047 |
| | protein 1 | |
| Q9BSJ8_C370 | ESYT1 Extended | 8048 |
| | synaptotagmin-1 | |
| P51610_C227 | HCFC1 Host cell factor 1 | 8049 |
| P49753_C401 | ACOT2 Acyl-coenzyme A | 8050 |
| | thioesterase 2, mitochondrial | |
| J3QR44_C430 | CDK11B Cyclin-dependent | 8051 |
| | kinase 11B | |
| P21127_C440 | CDK11B Cyclin-dependent | 8052 |
| | kinase 11B | |
| O43149_C69 | ZZEF1 Zinc finger ZZ-type | 8053 |
| | and EF-hand domain- | |
| | containing | |
| Q9NZB2_C531 | FAM120A Constitutive | 8054 |
| | coactivator of PPAR-gamma- | |
| | like protein 1 | |
| Q6PJT7_C261 | ZC3H14 Zinc finger CCCH | 8055 |
| | domain-containing protein 14 | |
| Q9Y613_C31 | FHOD1 FH1/FH2 domain- | 8056 |
| | containing protein 1 | |
| Q9Y4F9_C596 | FAM65B Protein FAM65B | 8057 |
| Q93084_C614 | ATP2A3 | 8058 |
| | Sarcoplasmic/endoplasmic | |
| | reticulum calcium ATPase | |
| P78527_C25 | PRKDC DNA-dependent | 8059 |
| | protein kinase catalytic subunit | |
| Q15027_C501 | ACAP1 Arf-GAP with coiled- | 8060 |
| | coil, ANK repeat and PH | |
| | domain 1 | |
| Q9H2M9_C1336 | RAB3GAP2 Rab3 GTPase- | 8061 |
| | activating protein non-catalytic | |
| | subunit | |
| P62857_C27 | RPS28 40S ribosomal protein | 8062 |
| | S28 | |
| P60981_C163 | DSTN Destrin | 8063 |
| Q96SK2_C158 | TMEM209 Transmembrane | 8064 |
| | protein 209 | |
| P15153_C178 | RAC2 Ras-related C3 | 8065 |
| | botulinum toxin substrate 2 | |
| P37802_C124 | TAGLN2 Transgelin-2 | 8066 |
| Q63HN8_C3554 | RNF213 E3 ubiquitin-protein | 8067 |
| | ligase RNF213 | |
| O94915_C888 | FRYL Protein furry homolog- | 8068 |
| | like | |
| Q9UPR0_C90 | PLCL2 Inactive phospholipase | 8069 |
| | C-like protein 2 | |
| Q96ME7_C324 | ZNF512 Zinc finger protein | 8070 |
| | 512 | |
| Q8IZ73_C431 | RPUSD2 RNA | 8071 |
| | pseudouridylate synthase | |
| | domain-containing protein | |
| Q5SNT2_C498 | TMEM201 Transmembrane | 8072 |
| | protein 201 | |
| Q14980_C1937 | NUMA1 Nuclear mitotic | 8073 |
| | apparatus protein 1 | |
| Q9BSD7_C184 | NTPCR Cancer-related | 8074 |
| | nucleoside-triphosphatase | |
| P56192_C441 | MARS Methionine--tRNA | 8075 |
| | ligase, cytoplasmic | |
| Q9BWW4_C80 | SSBP3 Single-stranded DNA- | 8076 |
| | binding protein 3 | |
| P62834_C139 | RAP1A Ras-related protein | 8077 |
| | Rap-1A | |
| P49448_C172 | GLUD2 Glutamate | 8078 |
| | dehydrogenase 2, | |
| | mitochondrial | |
| P00367_C172 | GLUD1 Glutamate | 8079 |
| | dehydrogenase 1, | |
| | mitochondrial | |
| P23396_C134 | RPS3 40S ribosomal protein | 8080 |
| | S3 | |
| O75947_C101 | ATP5H ATP synthase subunit | 8081 |
| | d, mitochondrial | |
| Q9HC38_C171 | GLOD4 Glyoxalase domain- | 8082 |
| | containing protein 4 | |
| O00233_C59 | PSMD9 26S proteasome non- | 8083 |
| | ATPase regulatory subunit 9 | |
| P14866_C472 | HNRNPL Heterogeneous | 8084 |
| | nuclear ribonucleoprotein L | |
| Q04206_C105 | RELA Transcription factor | 8085 |
| | p65 | |
| Q96FW1_C23 | OTUB1 Ubiquitin thioesterase | 8086 |
| | OTUB1 | |
| Q9UI30_C33 | TRMT112 tRNA | 8087 |
| | methyltransferase 112 | |
| | homolog | |
| O43815_C316 | STRN Striatin | 8088 |
| P08047_C755 | SP1 Transcription factor Sp1 | 8089 |
| P60174_C104 | TPI1 Triosephosphate | 8090 |
| | isomerase | |
| O75362_C286 | ZNF217 Zinc finger protein | 8091 |
| | 217 | |
| O60496_C36 | DOK2 Docking protein 2 | 8092 |
| P49792_C2791 | RANBP2 E3 SUMO-protein | 8093 |
| | ligase RanBP2 | |
| Q13428_C38 | TCOF1 Treacle protein | 8094 |
| Q8TD47_C41 | RPS4Y2 40S ribosomal | 8095 |
| | protein S4, Y isoform 2 | |
| P22090_C41 | RPS4Y1 40S ribosomal | 8096 |
| | protein S4, Y isoform 1 | |
| Q8NDH3_C504 | NPEPL1 Probable | 8097 |
| | aminopeptidase NPEPL1 | |
| Q96P11_C362 | NSUN5 Putative | 8098 |
| | methyltransferase NSUN5 | |
| Q8N3X1_C877 | FNBP4 Formin-binding | 8099 |
| | protein 4 | |
| Q92685_C21 | ALG3 Dol-P- | 8100 |
| | Man:Man(5)GlcNAc(2)-PP- | |
| | Dol alpha-1,3- | |
| | mannosyltransferase | |
| Q00013_C242 | MPP1 55 kDa erythrocyte | 8101 |
| | membrane protein | |
| Q9BTX1_C468 | TMEM48 Nucleoporin NDC1 | 8102 |
| Q96RU3_C609 | FNBP1 Formin-binding | 8103 |
| | protein 1 | |
| P31749_C310 | AKT1 RAC-alpha | 8104 |
| | serine/threonine-protein kinase | |
| Q9Y243_C307 | AKT3 RAC-gamma | 8105 |
| | serine/threonine-protein kinase | |
| P31751_C311 | AKT2 RAC-beta | 8106 |
| | serine/threonine-protein kinase | |
| O95248_C1374 | SBF1 Myotubularin-related | 8107 |
| | protein 5 | |
| O94953_C694 | KDM4B Lysine-specific | 8108 |
| | demethylase 4B | |
| O95295_C66 | SNAPIN SNARE-associated | 8109 |
| | protein Snapin | |
| Q9Y4W2_C690 | LAS1L Ribosomal biogenesis | 8110 |
| | protein LAS1L | |
| P29375_C838 | KDM5A Lysine-specific | 8111 |
| | demethylase 5A | |
| P52948_C1068 | NUP98 Nuclear pore complex | 8112 |
| | protein Nup98-Nup96 | |
| Q9Y5Y2_C269 | NUBP2 Cytosolic Fe—S cluster | 8113 |
| | assembly factor NUBP2 | |
| P12814_C370 | ACTN1 Alpha-actinin-1 | 8114 |
| O75131_C506 | CPNE3 Copine-3 | 8115 |
| Q7Z2W4_C38 | ZC3HAV1 Zinc finger CCCH- | 8116 |
| | type antiviral protein 1 | |
| P51610_C1886 | HCFC1 Host cell factor 1 | 8117 |
| P51610_C1895 | HCFC1 Host cell factor 1 | 8118 |
| P40939_C322 | HADHA Trifunctional enzyme | 8119 |
| | subunit alpha, mitochondrial | |
| Q63HN8_C3261 | RNF213 E3 ubiquitin-protein | 8120 |
| | ligase RNF213 | |
| I3L3Q1_C264 | Uncharacterized protein | 8121 |
| Q6ZSZ5_C306 | ARHGEF18 Rho guanine | 8122 |
| | nucleotide exchange factor 18 | |
| Q9BRJ7_C88 | NUDT16L1 Protein | 8123 |
| | syndesmos | |
| P52701_C615 | MSH6 DNA mismatch repair | 8124 |
| | protein Msh6 | |
| Q9Y4K1_C976 | AIM1 Absent in melanoma 1 | 8125 |
| | protein | |
| Q15631_C225 | TSN Translin | 8126 |
| Q9H9L4_C117 | KANSL2 KAT8 regulatory | 8127 |
| | NSL complex subunit 2 | |
| Q66K74_C435 | MAP1S Microtubule- | 8128 |
| | associated protein 1S | |
| Q66K74_C440 | MAP1S Microtubule- | 8129 |
| | associated protein 1S | |
| O75319_C325 | DUSP11 RNA/RNP complex- | 8130 |
| | 1-interacting phosphatase | |
| Q02543_C16 | RPL18A 60S ribosomal | 8131 |
| | protein L18a | |
| Q96HA1_C307 | POM121 Nuclear envelope | 8132 |
| | pore membrane protein POM | |
| | 121 | |
| Q6PJE2_C42 | POMZP3 POM121 and ZP3 | 8133 |
| | fusion protein | |
| A8CG34_C284 | POM121C Nuclear envelope | 8134 |
| | pore membrane protein POM | |
| | 121C | |
| O14523_C359 | C2CD2L C2 domain- | 8135 |
| | containing protein 2-like | |
| Q99986_C50 | VRK1 Serine/threonine- | 8136 |
| | protein kinase VRK1 | |
| P62995_C118 | TRA2B Transformer-2 protein | 8137 |
| | homolog beta | |
| P62995_C119 | TRA2B Transformer-2 protein | 8138 |
| | homolog beta | |
| Q15050_C52 | RRS1 Ribosome biogenesis | 8139 |
| | regulatory protein homolog | |
| P08575_C608 | PTPRC Receptor-type | 8140 |
| | tyrosine-protein phosphatase C | |
| O43704_C22 | SULT1B1 Sulfotransferase | 8141 |
| | family cytosolic 1B member 1 | |
| P46060_C573 | RANGAP1 Ran GTPase- | 8142 |
| | activating protein 1 | |
| E9PMF8_C542 | PTPRC Receptor-type | 8143 |
| | tyrosine-protein phosphatase C | |
| Q13469_C386 | NFATC2 Nuclear factor of | 8144 |
| | activated T-cells, cytoplasmic | |
| | 2 | |
| A6NHR9_C1710 | SMCHD1 Structural | 8145 |
| | maintenance of chromosomes | |
| | flexible hinge domain | |
| | containing 1 | |
| O43159_C451 | RRP8 Ribosomal RNA- | 8146 |
| | processing protein 8 | |
| Q99439_C274 | CNN2 Calponin-2 | 8147 |
| Q99439_C290 | CNN2 Calponin-2 | 8148 |
| Q06330_C397 | RBPJ Recombining binding | 8149 |
| | protein suppressor of hairless | |
| O43264_C568 | ZW10 | 8150 |
| | Centromere/kinetochore | |
| | protein zw10 homolog | |
| Q8N806_C35 | UBR7 Putative E3 ubiquitin- | 8151 |
| | protein ligase UBR7 | |
| Q9UGV2_C359 | NDRG3 Protein NDRG3 | 8152 |
| P08758_C316 | ANXA5 Annexin A5 | 8153 |
| Q92878_C1296 | RAD50 DNA repair protein | 8154 |
| | RAD50 | |
| B0V043_C112 | VARS Valyl-tRNA synthetase | 8155 |
| O94903_C261 | PROSC Proline synthase co- | 8156 |
| | transcribed bacterial homolog | |
| P53041_C11 | PPP5C Serine/threonine- | 8157 |
| | protein phosphatase 5 | |
| Q92608_C1655 | DOCK2 Dedicator of | 8158 |
| | cytokinesis protein 2 | |
| P35236_C62 | PTPN7 Tyrosine-protein | 8159 |
| | phosphatase non-receptor type | |
| | 7 | |
| Q86YV0_C896 | RASAL3 RAS protein | 8160 |
| | activator like-3 | |
| Q6P1X6_C98 | C8orf82 UPF0598 protein | 8161 |
| | C8orf82 | |
| Q8TDN6_C52 | BRIX1 Ribosome biogenesis | 8162 |
| | protein BRX1 homolog | |
| Q15424_C225 | SAFB Scaffold attachment | 8163 |
| | factor B1 | |
| Q14151_C224 | SAFB2 Scaffold attachment | 8164 |
| | factor B2 | |
| Q8WXH0_C6161 | SYNE2 Nesprin-2 | 8165 |
| P45880_C13 | VDAC2 Voltage-dependent | 8166 |
| | anion-selective channel protein | |
| Q92575_C144 | UBXN4 UBX domain- | 8167 |
| | containing protein 4 | |
| P07741_C83 | APRT Adenine | 8168 |
| | phosphoribosyltransferase | |
| Q9BWH6_C1039 | RPAP1 RNA polymerase II- | 8169 |
| | associated protein 1 | |
| Q96EP0_C551 | RNF31 E3 ubiquitin-protein | 8170 |
| | ligase RNF31 | |
| Q9BYG3_C237 | MKI67IP MKI67 FHA | 8171 |
| | domain-interacting nucleolar | |
| | phosphoprotein | |
| P17655_C39 | CAPN2 Calpain-2 catalytic | 8172 |
| | subunit | |
| Q12789_C42 | GTF3C1 General transcription | 8173 |
| | factor 3C polypeptide 1 | |
| O00410_C1078 | IPO5 Importin-5 | 8174 |
| Q15796_C70 | SMAD2 Mothers against | 8175 |
| | decapentaplegic homolog 2 | |
| Q9H7D7_C656 | WDR26 WD repeat-containing | 8176 |
| | protein 26 | |
| O94804_C947 | STK10 Serine/threonine- | 8177 |
| | protein kinase 10 | |
| A6NHR9_C59 | SMCHD1 Structural | 8178 |
| | maintenance of chromosomes | |
| | flexible hinge domain | |
| | containing 1 | |
| Q13501_C26 | SQSTM1 Sequestosome-1 | 8179 |
| Q13501_C27 | SQSTM1 Sequestosome-1 | 8180 |
| P04899_C352 | GNAI2 Guanine nucleotide- | 8181 |
| | binding protein G(i) subunit | |
| | alpha-2 | |
| P40222_C523 | TXLNA Alpha-taxilin | 8182 |
| Q6UUV9_C131 | CRTC1 CREB-regulated | 8183 |
| | transcription coactivator 1 | |
| O75643_C428 | SNRNP200 U5 small nuclear | 8184 |
| | ribonucleoprotein 200 kDa | |
| | helicas | |
| P29590_C338 | PML Protein PML | 8185 |
| Q32MZ4_C14 | LRRMP1 Leucine-rich repeat | 8186 |
| | flightless-interacting protein | |
| P07814_C1487 | EPRS Bifunctional | 8187 |
| | glutamate/proline--tRNA | |
| | ligase | |
| Q08999_C368 | RBL2 Retinoblastoma-like | 8188 |
| | protein 2 | |
| Q6IA86_C475 | ELP2 Elongator complex | 8189 |
| | protein 2 | |
| A6NHR9_C61 | SMCHD1 Structural | 8190 |
| | maintenance of chromosomes | |
| | flexible hinge domain | |
| | containing 1 | |
| P35611_C525 | ADD1 Alpha-adducin | 8191 |
| P52701_C88 | MSH6 DNA mismatch repair | 8192 |
| | protein Msh6 | |
| Q14137_C108 | BOP1 Ribosome biogenesis | 8193 |
| | protein BOP1 | |
| Q12802_C1232 | AKAP13 A-kinase anchor | 8194 |
| | protein 13 | |
| Q92800_C423 | EZH1 Histone-lysine N- | 8195 |
| | methyltransferase EZH1 | |
| Q8N9N8_C89 | EIF1AD Probable RNA- | 8196 |
| | binding protein EIF1AD | |
| Q13813_C2233 | SPTAN1 Spectrin alpha chain, | 8197 |
| | non-erythrocytic 1 | |
| Q96EK4_C48 | THAP11 THAP domain- | 8198 |
| | containing protein 11 | |
| O95267_C713 | RASGRP1 RAS guanyl- | 8199 |
| | releasing protein 1 | |
| P62851_C59 | RPS25 40S ribosomal protein | 8200 |
| | S25 | |
| O60551_C104 | NMT2 Glycylpeptide N- | 8201 |
| | tetradecanoyltransferase 2 | |
| Q9Y228_C135 | TRAF3IP3 TRAF3-interacting | 8202 |
| | JNK-activating modulator | |
| I3L1I5_C189 | Uncharacterized protein | 8203 |
| P08047_C68 | SP1 Transcription factor Sp1 | 8204 |
| E7ETI0_C21 | ARPC4-TTLL3 Protein | 8205 |
| | ARPC4-TTLL3 | |
| P59998_C21 | ARPC4 Actin-related protein | 8206 |
| | 2/3 complex subunit 4 | |
| Q7KZ85_C336 | SUPT6H Transcription | 8207 |
| | elongation factor SPT6 | |
| Q9HB58_C461 | SP110 Sp110 nuclear body | 8208 |
| | protein | |
| B0I1T2_C793 | MYO1G Unconventional | 8209 |
| | myosin-Ig | |
| Q99497_C106 | PARK7 Protein DJ-1 | 8210 |
| Q9HB19_C191 | PLEKHA2 Pleckstrin | 8211 |
| | homology domain-containing | |
| | family A member 2 | |
| B0I1T2_C788 | MYO1G Unconventional | 8212 |
| | myosin-Ig | |
| Q9NWY4_C17 | C4orf27 UPF0609 protein | 8213 |
| | C4orf27 | |
| Q9H4L4_C243 | SENP3 Sentrin-specific | 8214 |
| | protease 3 | |
| A0AVT1_C347 | UBA6 Ubiquitin-like | 8215 |
| | modifier-activating enzyme 6 | |
| P23497_C238 | SP100 Nuclear autoantigen | 8216 |
| | Sp-100 | |
| Q9NU22_C4041 | MDN1 Midasin | 8217 |
| Q92835_C506 | INPP5D Phosphatidylinositol | 8218 |
| | 3,4,5-trisphosphate 5- | |
| | phosphatase D | |
| Q66GS9_C316 | CEP135 Centrosomal protein | 8219 |
| | of 135 kDa | |
| P48739_C13 | PITPNB Phosphatidylinositol | 8220 |
| | transfer protein beta isoform | |
| P35914_C323 | HMGCL | 8221 |
| | Hydroxymethylglutaryl-CoA | |
| | lyase, mitochondrial | |
| Q5JSL3_C1190 | DOCK11 Dedicator of | 8222 |
| | cytokinesis protein 11 | |
| Q96JY6_C160 | PDLIM2 PDZ and LIM | 8223 |
| | domain protein 2 | |
| Q7Z5K2_C94 | WAPAL Wings apart-like | 8224 |
| | protein homolog | |
| Q00653_C57 | NFKB2 Nuclear factor NF- | 8225 |
| | kappa-B p100 subunit | |
| Q8TC07_C686 | TBC1D15 TBC1 domain | 8226 |
| | family member 15 | |
| Q8N1F7_C422 | NUP93 Nuclear pore complex | 8227 |
| | protein Nup93 | |
| P57678_C210 | GEMIN4 Gem-associated | 8228 |
| | protein 4 | |
| Q7Z5R6_C401 | APBB1IP Amyloid beta A4 | 8229 |
| | precursor protein-binding | |
| | family B | |
| Q9NX24_C18 | NHP2 H/ACA | 8230 |
| | ribonucleoprotein complex | |
| | subunit 2 | |
| P55265_C622 | ADAR Double-stranded RNA- | 8231 |
| | specific adenosine deaminase | |
| Q8WVV9_C464 | HNRPLL Heterogeneous | 8232 |
| | nuclear ribonucleoprotein L- | |
| | like | |
| Q99832_C364 | CCT7 T-complex protein 1 | 8233 |
| | subunit eta | |
| P63244_C286 | GNB2L1 Guanine nucleotide- | 8234 |
| | binding protein subunit beta-2- | |
| | like 1 | |
| Q13427_C310 | PPIG Peptidyl-prolyl cis-trans | 8235 |
| | isomerase G | |
| P48147_C25 | PREP Prolyl endopeptidase | 8236 |
| Q9UJZ1_C167 | STOML2 Stomatin-like | 8237 |
| | protein 2 | |
| O75369_C26 | FLNB Filamin-B | 8238 |
| P21333_C53 | FLNA Filamin-A | 8239 |
| Q92974_C335 | ARHGEF2 Rho guanine | 8240 |
| | nucleotide exchange factor 2 | |
| Q92835_C1050 | INPP5D Phosphatidylinositol | 8241 |
| | 3,4,5-trisphosphate 5- | |
| | phosphatase 1 | |
| Q4G0F5_C334 | VPS26B Vacuolar protein | 8242 |
| | sorting-associated protein 26B | |
| P17844_C234 | DDX5 Probable ATP- | 8243 |
| | dependent RNA helicase | |
| | DDX5 | |
| Q9BSD7_C101 | NTPCR Cancer-related | 8244 |
| | nucleoside-triphosphatase | |
| Q92619_C1020 | HMHA1 Minor | 8245 |
| | histocompatibility protein HA- | |
| | 1 | |
| Q14790_C360 | CASP8 Caspase-8 | 8246 |
| O15067_C1285 | PFAS | 8247 |
| | Phosphoribosylformylglycina | |
| | midine synthase | |
| O15067_C1287 | PFAS | 8248 |
| | Phosphoribosylformylglycina | |
| | midine synthase | |
| O60880_C124 | SH2D1A SH2 domain- | 8249 |
| | containing protein 1A | |
| Q92888_C537 | ARHGEF1 Rho guanine | 8250 |
| | nucleotide exchange factor 1 | |
| O14879_C343 | IFIT3 Interferon-induced | 8251 |
| | protein with tetratricopeptide | |
| Q93052_C364 | LPP Lipoma-preferred partner | 8252 |
| P22695_C192 | UQCRC2 Cytochrome b-c1 | 8253 |
| | complex subunit 2, | |
| | mitochondrial | |
| P10398_C597 | ARAF Serine/threonine- | 8254 |
| | protein kinase A-Raf | |
| P78347_C215 | GTF2I General transcription | 8255 |
| | factor II-I | |
| P08575_C1259 | PTPRC Receptor-type | 8256 |
| | tyrosine-protein phosphatase C | |
| Q9UHB9_C562 | SRP68 Signal recognition | 8257 |
| | particle 68 kDa protein | |
| P40121_C290 | CAPG Macrophage-capping | 8258 |
| | protein | |
| P36578_C250 | RPL4 60S ribosomal protein | 8259 |
| | L4 | |
| A9UHW6_C49 | MIF4GD MIF4G domain- | 8260 |
| | containing protein | |
| Q9Y3B4_C83 | SF3B14 Pre-mRNA branch | 8261 |
| | site protein p14 | |
| Q93008_C1212 | USP9X Probable ubiquitin | 8262 |
| | carboxyl-terminal hydrolase | |
| | FAF | |
| P11171_C179 | EPB41 Protein 4.1 | 8263 |
| Q9BZS1_C169 | FOXP3 Forkhead box protein | 8264 |
| | P3 | |
| Q13469_C256 | NFATC2 Nuclear factor of | 8265 |
| | activated T-cells, cytoplasmic | |
| | 2 | |
| Q9BWW8_C25 | APOL6 Apolipoprotein L6 | 8266 |
| Q15366_C217 | PCBP2 Poly(rC)-binding | 8267 |
| | protein 2 | |
| P13796_C101 | LCP1 Plastin-2 | 8268 |
| Q16851_C123 | UGP2 UTP--glucose-1- | 8269 |
| | phosphate uridylyltransferase | |
| P53602_C108 | MVD Diphosphomevalonate | 8270 |
| | decarboxylase | |
| Q9NR30_C378 | DDX21 Nucleolar RNA | 8271 |
| | helicase 2 | |
| P13727_C201 | PRG2 Bone marrow | 8272 |
| | proteoglycan | |
| O14867_C646 | BACH1 Transcription | 8273 |
| | regulator protein BACH1 | |
| O43772_C283 | SLC25A20 Mitochondrial | 8274 |
| | camitine/acylcamitine carrier | |
| | protein | |
| Q9Y3F4_C305 | STRAP Serine-threonine | 8275 |
| | kinase receptor-associated | |
| | protein | |
| Q12802_C1677 | AKAP13 A-kinase anchor | 8276 |
| | protein 13 | |
| Q9BSU1_C222 | C16orf70 UPF0183 protein | 8277 |
| | C16orf70 | |
| P78362_C320 | SRPK2 SRSF protein kinase 2 | 8278 |
| Q9NZJ7_C385 | MTCH1 Mitochondrial carrier | 8279 |
| | homolog 1 | |
| P51692_C688 | STAT5B Signal transducer | 8280 |
| | and activator of transcription 5 | |
| Q04637_C662 | EIF4G1 Eukaryotic translation | 8281 |
| | initiation factor 4 gamma 1 | |
| Q08AF3_C369 | SLFN5 Schlafen family | 8282 |
| | member 5 | |
| Q9NZB2_C919 | FAM120A Constitutive | 8283 |
| | coactivator of PPAR-gamma- | |
| | like protein 1 | |
| O60684_C238 | KPNA6 Importin subunit | 8284 |
| | alpha-7 | |
| O15131_C238 | KPNA5 Importin subunit | 8285 |
| | alpha-6 | |
| P52294_C240 | KPNA1 Importin subunit | 8286 |
| | alpha-1 | |
| Q8ND71_C214 | GIMAP8 GTPase IMAP | 8287 |
| | family member 8 | |
| P35658_C728 | NUP214 Nuclear pore | 8288 |
| | complex protein Nup214 | |
| Q32MZ4_C644 | LRRFIP1 Leucine-rich repeat | 8289 |
| | flightless-interacting protein | |
| Q8TCU6_C1116 | PREX1 Phosphatidylinositol | 8290 |
| | 3,4,5-trisphosphate-dependent | |
| Q96I24_C460 | FUBP3 Far upstream element- | 8291 |
| | binding protein 3 | |
| Q6KC79_C1066 | NIPBL Nipped-B-like protein | 8292 |
| Q03519_C353 | TAP2 Antigen peptide | 8293 |
| | transporter 2 | |
| Q7Z3K3_C547 | POGZ Pogo transposable | 8294 |
| | element with ZNF domain | |
| Q7L8W6_C88 | ATPBD4 ATP-binding | 8295 |
| | domain-containing protein 4 | |
| Q9Y2Z0_C62 | SUGT1 Suppressor of G2 | 8296 |
| | allele of SKP1 homolog | |
| P34932_C376 | HSPA4 Heat shock 70 kDa | 8297 |
| | protein 4 | |
| Q92598_C376 | HSPH1 Heat shock protein | 8298 |
| | 105 kDa | |
| P17844_C221 | DDX5 Probable ATP- | 8299 |
| | dependent RNA helicase | |
| | DDX5 | |
| Q92841_C298 | DDX17 Probable ATP- | 8300 |
| | dependent RNA helicase | |
| | DDX17 | |
| Q5JSL3_C1204 | DOCK11 Dedicator of | 8301 |
| | cytokinesis protein 11 | |
| P09564_C230 | CD7 T-cell antigen CD7 | 8302 |
| P27635_C105 | RPL10 60S ribosomal protein | 8303 |
| | L10 | |
| Q9UKA8_C173 | RCAN3 Calcipressin-3 | 8304 |
| P00492_C106 | HPRT1 Hypoxanthine-guanine | 8305 |
| | phosphoribosyltransferase | |
| P26038_C117 | MSN Moesin | 8306 |
| P12955_C482 | PEPD Xaa-Pro dipeptidase | 8307 |
| Q9NR33_C84 | POLE4 DNA polymerase | 8308 |
| | epsilon subunit 4 | |
| Q9H8W4_C219 | PLEKHF2 Pleckstrin | 8309 |
| | homology domain-containing | |
| | family F member | |
| Q8WYP5_C1131 | AHCTF1 Protein ELYS | 8310 |
| Q9Y6K5_C350 | OAS3 2-5-oligoadenylate | 8311 |
| | synthase 3 | |
| P41250_C616 | GARS Glycine--tRNA ligase | 8312 |
| Q8NF50_C143 | DOCK8 Dedicator of | 8313 |
| | cytokinesis protein 8 | |
| H3BQZ7_C57 | Uncharacterized protein | 8314 |
| Q1KMD3_C57 | HNRNPUL2 Heterogeneous | 8315 |
| | nuclear ribonucleoprotein U- | |
| | like protein | |
| Q5JTH9_C102 | RRP12 RRP12-like protein | 8316 |
| Q9C0C9_C910 | UBE2O Ubiquitin-conjugating | 8317 |
| | enzyme E2 O | |
| Q9C0C9_C913 | UBE2O Ubiquitin-conjugating | 8318 |
| | enzyme E2 O | |
| Q8NF50_C197 | DOCK8 Dedicator of | 8319 |
| | cytokinesis protein 8 | |
| O95466_C45 | FMNL1 Formin-like protein 1 | 8320 |
| Q53GL7_C50 | PARP10 Polymerase | 8321 |
| Q9P2R3_C675 | ANKFY1 Ankyrin repeat and | 8322 |
| | FYVE domain-containing | |
| | protein | |
| O75607_C105 | NPM3 Nucleoplasmin-3 | 8323 |
| Q9NXA8_C303 | SIRT5 NAD-dependent | 8324 |
| | protein deacylase sirtuin-5, | |
| | mitochondrial | |
| Q00013_C454 | MPP1 55 kDa erythrocyte | 8325 |
| | membrane protein | |
| O60701_C276 | UGDH UDP-glucose 6- | 8326 |
| | dehydrogenase | |
| Q15149_C730 | PLEC Plectin | 8327 |
| Q9Y4P8_C393 | WIPI2 WD repeat domain | 8328 |
| | phosphoinositide-interacting | |
| | protein | |
| P27816_C126 | MAP4 Microtubule-associated | 8329 |
| | protein 4 | |
| O95487_C87 | SEC24B Protein transport | 8330 |
| | protein Sec24B | |
| Q3SXM5_C265 | HSDL1 Inactive | 8331 |
| | hydroxysteroid | |
| | dehydrogenase-like protein | |
| H3BQZ7_C293 | Uncharacterized protein | 8332 |
| Q1KMD3_C293 | HNRNPUL2 Heterogeneous | 8333 |
| | nuclear ribonucleoprotein U- | |
| | like protein 2 | |
| P16871_C349 | IL7R Interleukin-7 receptor | 8334 |
| | subunit alpha | |
| Q9HB90_C377 | RRAGC Ras-related GTP- | 8335 |
| | binding protein C | |
| Q14919_C54 | DRAP1 Dr1-associated | 8336 |
| | corepressor | |
| Q86YS7_C867 | KIAA0528 Uncharacterized | 8337 |
| | protein KIAA0528 | |
| P49915_C456 | GMPS GMP synthase | 8338 |
| Q13428_C1298 | TCOF1 Treacle protein | 8339 |
| Q9H9A6_C264 | LRRC40 Leucine-rich repeat- | 8340 |
| | containing protein 40 | |
| Q12802_C1548 | AKAP13 A-kinase anchor | 8341 |
| | protein 13 | |
| Q06330_C313 | RBPJ Recombining binding | 8342 |
| | protein suppressor of hairless | |
| Q969E8_C17 | TSR2 Pre-rRNA-processing | 8343 |
| | protein TSR2 homolog | |
| Q9ULT8_C254 | HECTD1 E3 ubiquitin-protein | 8344 |
| | ligase HECTD1 | |
| P51858_C12 | HDGF Hepatoma-derived | 8345 |
| | growth factor | |
| Q99543_C394 | DNAJC2 DnaJ homolog | 8346 |
| | subfamily C member 2 | |
| P12268_C331 | IMPDH2 Inosine-5- | 8347 |
| | monophosphate | |
| | dehydrogenase 2 | |
| P42166_C341 | TMPO Lamina-associated | 8348 |
| | polypeptide 2, isoform alpha | |
| Q92888_C911 | ARHGEF1 Rho guanine | 8349 |
| | nucleotide exchange factor 1 | |
| Q9BZ29_C628 | DOCK9 Dedicator of | 8350 |
| | cytokinesis protein 9 | |
| O95336_C78 | PGLS 6- | 8351 |
| | phosphogluconolactonase | |
| Q9UEW8_C525 | STK39 STE20/SPS1-related | 8352 |
| | proline-alanine-rich protein | |
| | kinase | |
| Q63HN8_C614 | RNF213 E3 ubiquitin-protein | 8353 |
| | ligase RNF213 | |
| P49023_C108 | PXN Paxillin | 8354 |
| Q16666_C679 | IFI16 Gamma-interferon- | 8355 |
| | inducible protein 16 | |
| Q9Y5Y2_C196 | NUBP2 Cytosolic Fe—S cluster | 8356 |
| | assembly factor NUBP2 | |
| Q9Y5Y2_C202 | NUBP2 Cytosolic Fe—S cluster | 8357 |
| | assembly factor NUBP2 | |
| Q9GZR7_C49 | DDX24 ATP-dependent RNA | 8358 |
| | helicase DDX24 | |
| Q96F63_C78 | CCDC97 Coiled-coil domain- | 8359 |
| | containing protein 97 | |
| Q9Y5Y2_C199 | NUBP2 Cytosolic Fe—S cluster | 8360 |
| | assembly factor NUBP2 | |
| Q96F07_C1112 | CYFIP2 Cytoplasmic FMR1- | 8361 |
| | interacting protein 2 | |
| Q9UPU5_C153 | USP24 Ubiquitin carboxyl- | 8362 |
| | terminal hydrolase 24 | |
| P02545_C570 | LMNA Prelamin-A/C | 8363 |
| Q7L576_C1088 | CYF1P1 Cytoplasmic FMR1- | 8364 |
| | interacting protein 1 | |
| O95671_C333 | ASMTL N-acetylserotonin O- | 8365 |
| | methyltransferase-like protein | |
| Q7Z6B0_C424 | CCDC91 Coiled-coil domain- | 8366 |
| | containing protein 91 | |
| P49321_C708 | NASP Nuclear autoantigenic | 8367 |
| | sperm protein | |
| P61978_C132 | HNRNPK Heterogeneous | 8368 |
| | nuclear ribonucleoprotein K | |
| P42224_C255 | STAT1 Signal transducer and | 8369 |
| | activator of transcription 1 | |
| Q01518_C427 | CAP1 Adenylyl cyclase- | 8370 |
| | associated protein 1 | |
| O94874_C32 | UFL1 E3 UFM1-protein ligase | 8371 |
| | 1 | |
| P42224_C247 | STAT1 Signal transducer and | 8372 |
| | activator of transcription 1 | |
| Q96BY6_C2164 | DOCK10 Dedicator of | 8373 |
| | cytokinesis protein 10 | |
| O43586_C305 | PSTPIP1 Proline-serine- | 8374 |
| | threonine phosphatase- | |
| | interacting protein 1 | |
| P50851_C1704 | LRBA Lipopolysaccharide- | 8375 |
| | responsive and beige-like | |
| | anchor protein | |
| Q96QR8_C17 | PURB Transcriptional | 8376 |
| | activator protein Pur-beta | |
| Q9H3H3_C222 | C11orf68 UPF0696 protein | 8377 |
| | C11orf68 | |
| Q14997_C1840 | PSME4 Proteasome activator | 8378 |
| | complex subunit 4 | |
| Q9BUP3_C172 | HTATIP2 Oxidoreductase | 8379 |
| | HTATIP2 | |
| Q5VSL9_C769 | FAM40A Protein FAM40A | 8380 |
| Q9NW68_C49 | BSDC1 BSD domain- | 8381 |
| | containing protein 1 | |
| Q96F07_C1265 | CYFIP2 Cytoplasmic FMR1- | 8382 |
| | interacting protein 2 | |
| P60468_C39 | SEC61B Protein transport | 8383 |
| | protein Sec61 subunit beta | |
| Q15061_C380 | WDR43 WD repeat-containing | 8384 |
| | protein 43 | |
| Q04759_C322 | PRKCQ Protein kinase C theta | 8385 |
| | type | |
| Q13615_C826 | MTMR3 Myotubularin-related | 8386 |
| | protein 3 | |
| Q13630_C116 | TSTA3 GDP-L-fucose | 8387 |
| | synthase | |
| Q9Y6R4_C1604 | MAP3K4 Mitogen-activated | 8388 |
| | protein kinase kinase kinase 4 | |
| P49711_C155 | CTCF Transcriptional | 8389 |
| | repressor CTCF | |
| Q9UPS6_C1405 | SETD1B Histone-lysine N- | 8390 |
| | methyltransferase SETD1B | |
| Q8WVT3_C160 | TRAPPC12 Trafficking | 8391 |
| | protein particle complex | |
| | subunit 12 | |
| P55060_C842 | CSE1L Exportin-2 | 8392 |
| Q9BSJ8_C611 | ESYT1 Extended | 8393 |
| | synaptotagmin-1 | |
| P52948_C1027 | NUP98 Nuclear pore complex | 8394 |
| | protein Nup98-Nup96 | |
| P50991_C120 | CCT4 T-complex protein 1 | 8395 |
| | subunit delta | |
| Q13107_C702 | USP4 Ubiquitin carboxyl- | 8396 |
| | terminal hydrolase 4 | |
| P22681_C508 | CBL E3 ubiquitin-protein | 8397 |
| | ligase CBL | |
| Q08117_C26 | AES Amino-terminal enhancer | 8398 |
| | of split | |
| P19784_C336 | CSNK2A2 Casein kinase II | 8399 |
| | subunit alpha | |
| Q16576_C97 | RBBP7 Histone-binding | 8400 |
| | protein RBBP7 | |
| Q8NBF2_C716 | NHLRC2 NHL repeat- | 8401 |
| | containing protein 2 | |
| Q00403_C223 | GTF2B Transcription | 8402 |
| | initiation factor IIB | |
| P46527_C148 | CDKN1B Cyclin-dependent | 8403 |
| | kinase inhibitor 1B | |
| P17844_C191 | DDX5 Probable ATP- | 8404 |
| | dependent RNA helicase | |
| | DDX5 | |
| P22061_C102 | PCMT1 Protein-L- | 8405 |
| | isoaspartate(D-aspartate) O- | |
| | methyltransferase | |
| P35611_C430 | ADD1 Alpha-adducin | 8406 |
| P52888_C682 | THOP1 Thimet oligopeptidase | 8407 |
| Q01081_C67 | U2AF1 Splicing factor U2AF | 8408 |
| | 35 kDa subunit | |
| Q96BY9_C320 | TMEM66 Store-operated | 8409 |
| | calcium entry-associated | |
| | regulatory | |
| Q14152_C78 | EIF3A Eukaryotic translation | 8410 |
| | initiation factor 3 subunit | |
| P13489_C409 | RNH1 Ribonuclease inhibitor | 8411 |
| Q07960_C91 | ARHGAP1 Rho GTPase- | 8412 |
| | activating protein 1 | |
| Q9UGP4_C305 | LIMD1 LIM domain- | 8413 |
| | containing protein 1 | |
| P27816_C535 | MAP4 Microtubule-associated | 8414 |
| | protein 4 | |
| Q8NCE2_C182 | MTMR14 Myotubularin- | 8415 |
| | related protein 14 | |
| O00273_C154 | DFFA DNA fragmentation | 8416 |
| | factor subunit alpha | |
| Q92973_C153 | TNPO1 Transportin-1 | 8417 |
| P51608_C413 | MECP2 Methyl-CpG-binding | 8418 |
| | protein 2 | |
| Q63HN8_C3008 | RNF213 E3 ubiquitin-protein | 8419 |
| | ligase RNF213 | |
| O15294_C620 | OGT UDP-N- | 8420 |
| | acetylglucosamine--peptide N- | |
| | acetylglucosaminyl transferase | |
| P42166_C561 | TMPO Lamina-associated | 8421 |
| | polypeptide 2, isoform alpha | |
| Q6F5E8_C746 | RLTPR Leucine-rich repeat- | 8422 |
| | containing protein 16C | |
| P55265_C392 | ADAR Double-stranded RNA- | 8423 |
| | specific adenosine deaminase | |
| Q8NB37_C154 | PDDC1 Parkinson disease 7 | 8424 |
| | domain-containing protein 1 | |
| Q6JBY9_C155 | RCSD1 CapZ-interacting | 8425 |
| | protein | |
| Q9UJU2_C321 | LEF1 Lymphoid enhancer- | 8426 |
| | binding factor 1 | |
| O00186_C501 | STXBP3 Syntaxin-binding | 8427 |
| | protein 3 | |
| Q96BY6_C1310 | DOCK10 Dedicator of | 8428 |
| | cytokinesis protein 10 | |
| O00186_C498 | STXBP3 Syntaxin-binding | 8429 |
| | protein 3 | |
| P63104_C25 | YWHAZ 14-3-3 protein | 8430 |
| | zeta/delta | |
| Q8WU79_C196 | SMAP2 Stromal membrane- | 8431 |
| | associated protein 2 | |
| Q00765_C18 | REEP5 Receptor expression- | 8432 |
| | enhancing protein 5 | |
| P51608_C429 | MECP2 Methyl-CpG-binding | 8433 |
| | protein 2 | |
| O60841_C749 | EIF5B Eukaryotic translation | 8434 |
| | initiation factor 5B | |
| P42331_C524 | ARHGAP25 Rho GTPase- | 8435 |
| | activating protein 25 | |
| P35579_C988 | MYH9 Myosin-9 | 8436 |
| Q14151_C672 | SAFB2 Scaffold attachment | 8437 |
| | factor B2 | |
| Q99490_C811 | AGAP2 Arf-GAP with | 8438 |
| | GTPase, ANK repeat and PH | |
| | domain-containing protein 2 | |
| O60307_C762 | MAST3 Microtubule- | 8439 |
| | associated serine/threonine- | |
| | protein ki | |
| P27707_C9 | DCK Deoxycytidine kinase | 8440 |
| Q99704_C308 | DOK1 Docking protein 1 | 8441 |
| O75940_C214 | SMNDC1 Survival of motor | 8442 |
| | neuron-related-splicing factor | |
| | 3 | |
| O15269_C438 | SPTLC1 Serine | 8443 |
| | palmitoyltransferase 1 | |
| Q5EBM0_C119 | CMPK2 UMP-CMP kinase 2, | 8444 |
| | mitochondrial | |
| Q96F86_C137 | EDC3 Enhancer of mRNA- | 8445 |
| | decapping protein 3 | |
| Q86YV0_C1005 | RASAL3 RAS protein | 8446 |
| | activator like-3 | |
| P30044_C204 | PRDX5 Peroxiredoxin-5, | 8447 |
| | mitochondrial | |
| P78527_C1904 | PRKDC DNA-dependent | 8448 |
| | protein kinase catalytic subunit | |
| Q9BXP2_C911 | SLC12A9 Solute carrier | 8449 |
| | family 12 member 9 | |
| Q9Y6G9_C51 | DYNC1LI1 Cytoplasmic | 8450 |
| | dynein 1 light intermediate | |
| | chain 1 | |
| Q12962_C174 | TAF10 Transcription initiation | 8451 |
| | factor TFIID subunit 10 | |
| Q8IV53_C412 | DENND1C DENN domain- | 8452 |
| | containing protein 1C | |
| Q8NFW8_C364 | CMAS N-acylneuraminate | 8453 |
| | cytidylyltransferase | |
| Q7Z4S6_C299 | KIF21A Kinesin-like protein | 8454 |
| | KIF21A | |
| Q9Y383_C348 | LUC7L2 Putative RNA- | 8455 |
| | binding protein Luc7-like 2 | |
| Q12768_C21 | KLAA0196 WASH complex | 8456 |
| | subunit strumpellin | |
| Q9ULX6_C211 | AKAP8L A-kinase anchor | 8457 |
| | protein 8-like | |
| Q2NKJ3_C584 | CTC1 CST complex subunit | 8458 |
| | CTC1 | |
| Q7Z4W1_C138 | DCXR L-xylulose reductase | 8459 |
| P78527_C1507 | PRKDC DNA-dependent | 8460 |
| | protein kinase catalytic subunit | |
| Q9NQC3_C1101 | RTN4 Reticulon-4 | 8461 |
| Q8NCE2_C429 | MTMR14 Myotubularin- | 8462 |
| | related protein 14 | |
| P41227_C194 | NAA10 N-alpha- | 8463 |
| | acetyltransferase 10 | |
| Q08AF3_C846 | SLFN5 Schlafen family | 8464 |
| | member 5 | |
| Q9NXG2_C31 | THUMPD1 THUMP domain- | 8465 |
| | containing protein 1 | |
| Q00722_C945 | PLCB2 1-phosphatidylinositol | 8466 |
| | 4,5-bisphosphate | |
| | phosphodiesterase beta-2 | |
| Q14694_C94 | USP10 Ubiquitin carboxyl- | 8467 |
| | terminal hydrolase 10 | |
| Q12933_C287 | TRAF2 TNF receptor- | 8468 |
| | associated factor 2 | |
| P14735_C974 | IDE Insulin-degrading enzyme | 8469 |
| P32456_C405 | GBP2 Interferon-induced | 8470 |
| | guanylate-binding protein 2 | |
| P32456_C404 | GBP2 Interferon-induced | 8471 |
| | guanylate-binding protein 2 | |
| Q6PJG6_C539 | BRAT1 BRCA1-associated | 8472 |
| | ATM activator 1 | |
| Q9NPG8_C337 | ZDHHC4 Probable | 8473 |
| | palmitoyltransferase ZDHHC4 | |
| Q9Y2K7_C840 | KDM2A Lysine-specific | 8474 |
| | demethylase 2A | |
| P12081_C507 | HARS Histidine--tRNA ligase, | 8475 |
| | cytoplasmic | |
| Q13469_C231 | NFATC2 Nuclear factor of | 8476 |
| | activated T-cells, cytoplasmic | |
| | 2 | |
| Q16611_C14 | BAK1 Bcl-2 homologous | 8477 |
| | antagonist/killer | |
| P35579_C931 | MYH9 Myosin-9 | 8478 |
| Q9UFW8_C92 | CGGBP1 CGG triplet repeat- | 8479 |
| | binding protein 1 | |
| P11171_C49 | EPB41 Protein 4.1 | 8480 |
| Q9UQ35_C2116 | SRRM2 Serine/arginine | 8481 |
| | repetitive matrix protein 2 | |
| Q8WXI9_C308 | GATAD2B Transcriptional | 8482 |
| | repressor p66-beta | |
| Q96EY8_C132 | MMAB Cob(I)yrinic acid a,c- | 8483 |
| | diamide adenosyltransferase, | |
| | mitochondrial | |
| Q01433_C107 | AMPD2 AMP deaminase 2 | 8484 |
| Q9H0L4_C150 | CSTF2T Cleavage stimulation | 8485 |
| | factor subunit 2 tau variant | |
| P16188_C363 | HLA-A HLA class I histocompatibility | 8486 |
| | antigen, A-30 alpha | |
| P04439_C363 | HLA-A HLA class I histocompatibility | 8487 |
| | antigen, A-3 alpha | |
| P30443_C363 | HLA-A HLA class I histocompatibility | 8488 |
| | antigen, A-1 alpha | |
| O75663_C87 | TIPRL TIP41-like protein | 8489 |
| P19838_C61 | NFKB1 Nuclear factor NF- | 8490 |
| | kappa-B p105 subunit | |
| P07858_C319 | CTSB Cathepsin B | 8491 |
| O60502_C596 | MGEA5 Bifunctional protein | 8492 |
| | NCOAT | |
| Q9HB58_C283 | SP110 Sp110 nuclear body | 8493 |
| | protein | |
| P21333_C2543 | FLNA Filamin-A | 8494 |
| Q9BTE6_C209 | AARSD1 Alanyl-tRNA | 8495 |
| | editing protein Aarsd1 | |
| Q04864_C524 | REL Proto-oncogene c-Rel | 8496 |
| O75400_C39 | PRPF40A Pre-mRNA- | 8497 |
| | processing factor 40 homolog | |
| | A | |
| P04150_C367 | NR3C1 Glucocorticoid | 8498 |
| | receptor | |
| P33241_C283 | LSP1 Lymphocyte-specific | 8499 |
| | protein 1 | |
| O60934_C487 | NBN Nibrin | 8500 |
| Q86W50_C448 | METTL16 Methyltransferase- | 8501 |
| | like protein 16 | |
| Q9UQ35_C872 | SRRM2 Serine/arginine | 8502 |
| | repetitive matrix protein 2 | |
| Q8NDH3_C81 | NPEPL1 Probable | 8503 |
| | aminopeptidase NPEPL1 | |
| O43815_C765 | STRN Striatin | 8504 |
| O15145_C162 | ARPC3 Actin-related protein | 8505 |
| | 2/3 complex subunit 3 | |
| O15355_C164 | PPM1G Protein phosphatase | 8506 |
| | 1G | |
| H3BQZ7_C308 | Uncharacterized protein | 8507 |
| Q1KMD3_C308 | HNRNPUL2 Heterogeneous | 8508 |
| | nuclear ribonucleoprotein U- | |
| | like protein | |
| Q9NQ88_C114 | TIGAR Fructose-2,6- | 8509 |
| | bisphosphatase TIGAR | |
| P23497_C309 | SP100 Nuclear autoantigen | 8510 |
| | Sp-100 | |
| A2A288_C367 | ZC3H12D Probable | 8511 |
| | ribonuclease ZC3H12D | |
| Q9C0B6_C491 | FAM5B Protein FAM5B | 8512 |
| Q9UJW0_C258 | DCTN4 Dynactin subunit 4 | 8513 |
| P05771_C217 | PRKCB Protein kinase C beta | 8514 |
| | type | |
| Q96RS6_C376 | NUDCD1 NudC domain- | 8515 |
| | containing protein 1 | |
| Q8TD19_C878 | NEK9 Serine/threonine- | 8516 |
| | protein kinase Nek9 | |
| O95786_C680 | DDX58 Probable ATP- | 8517 |
| | dependent RNA helicase | |
| | DDX58 | |
| O94763_C44 | URI1 Unconventional | 8518 |
| | prefoldin RPB5 interactor 1 | |
| Q6PCB5_C280 | RSBN1L Round spermatid | 8519 |
| | basic protein 1-like protein | |
| Q09666_C1833 | AHNAK Neuroblast | 8520 |
| | differentiation-associated | |
| | protein AHNA | |
| O95861_C42 | BPNT1 3(2),5-bisphosphate | 8521 |
| | nucleotidase 1 | |
| Q96B23_C57 | C18orf25 Uncharacterized | 8522 |
| | protein C18orf25 | |
| Q9UER7_C699 | DAXX Death domain- | 8523 |
| | associated protein 6 | |
| Q7Z6I6_C954 | ARHGAP30 Rho GTPase- | 8524 |
| | activating protein 30 | |
| Q9P107_C335 | GMIP GEM-interacting | 8525 |
| | protein | |
| Q06323_C22 | PSME1 Proteasome activator | 8526 |
| | complex subunit 1 | |
| Q9P107_C336 | GMIP GEM-interacting | 8527 |
| | protein | |
| P35579_C569 | MYH9 Myosin-9 | 8528 |
| P13489_C75 | RNH1 Ribonuclease inhibitor | 8529 |
| Q9NY27_C22 | PPP4R2 Serine/threonine- | 8530 |
| | protein phosphatase 4 | |
| | regulatory | |
| Q8NFH5_C255 | NUP35 Nucleoporin NUP53 | 8531 |
| P13489_C85 | RNH1 Ribonuclease inhibitor | 8532 |
| Q00610_C870 | CLTC Clathrin heavy chain 1 | 8533 |
| O95071_C2267 | UBR5 E3 ubiquitin-protein | 8534 |
| | ligase UBR5 | |
| Q6NZY4_C393 | ZCCHC8 Zinc finger CCHC | 8535 |
| | domain-containing protein 8 | |
| A6QL63_C783 | BTBD11 Ankyrin repeat and | 8536 |
| | BTB/POZ domain-containing | |
| | protein | |
| Q09666_C1900 | AHNAK Neuroblast | 8537 |
| | differentiation-associated | |
| | protein AHNA | |
| Q09161_C44 | NCBP1 Nuclear cap-binding | 8538 |
| | protein subunit 1 | |
| Q5JSP0_C43 | FGD3 FYVE, RhoGEF and | 8539 |
| | PH domain-containing protein | |
| | 3 | |
| Q14839_C1594 | CHD4 Chromodomain- | 8540 |
| | helicase-DNA-binding protein | |
| | 4 | |
| Q16566_C202 | CAMK4 Calcium/calmodulin- | 8541 |
| | dependent protein kinase type | |
| | 1 | |
| Q8WTW3_C513 | COG1 Conserved oligomeric | 8542 |
| | Golgi complex subunit 1 | |
| Q09666_C2162 | AHNAK Neuroblast | 8543 |
| | differentiation-associated | |
| | protein AHNA | |
| Q9HA64_C24 | FN3KRP Ketosamine-3-kinase | 8544 |
| Q96E39_C338 | RBMXL1 RNA binding motif | 8545 |
| | protein, X-linked-like-1 | |
| P35610_C92 | SOAT1 Sterol O- | 8546 |
| | acyltransferase 1 | |
| Q96T76_C549 | MMS19 MMS19 nucleotide | 8547 |
| | excision repair protein | |
| | homolog | |
| Q9H4A3_C547 | WNK1 Serine/threonine- | 8548 |
| | protein kinase WNK1 | |
| Q7Z2T5_C320 | TRMT1L TRMT1-like protein | 8549 |
| Q99996_C3868 | AKAP9 A-kinase anchor | 8550 |
| | protein 9 | |
| Q96RU3_C70 | FNBP1 Formin-binding | 8551 |
| | protein 1 | |
| Q9H9A6_C54 | LRRC40 Leucine-rich repeat- | 8552 |
| | containing protein 40 | |
| O75569_C106 | PRKRA Interferon-inducible | 8553 |
| | double stranded RNA- | |
| | dependent | |
| P04150_C302 | NR3C1 Glucocorticoid | 8554 |
| | receptor | |
| Q9NUQ9_C10 | FAM49B Protein FAM49B | 8555 |
| P20592_C707 | MX2 Interferon-induced GTP- | 8556 |
| | binding protein Mx2 | |
| Q8N5K1_C92 | CISD2 CDGSH iron-sulfur | 8557 |
| | domain-containing protein 2 | |
| O14497_C336 | ARID1A AT-rich interactive | 8558 |
| | domain-containing protein 1A | |
| O75694_C874 | NUP155 Nuclear pore | 8559 |
| | complex protein Nup155 | |
| Q5W0V3_C298 | FAM160B1 Protein | 8560 |
| | FAM160B1 | |
| Q9NSD9_C195 | FARSB Phenylalanine--tRNA | 8561 |
| | ligase beta subunit | |
| P45984_C177 | MAPK9 Mitogen-activated | 8562 |
| | protein kinase 9 | |
| Q92835_C956 | INPP5D Phosphatidylinositol | 8563 |
| | 3,4,5-trisphosphate 5- | |
| | phosphatase | |
| Q96EV2_C726 | RBM33 RNA-binding protein | 8564 |
| | 33 | |
| P55198_C571 | MLLT6 Protein AF-17 | 8565 |
| O75475_C204 | PSIP1 PC4 and SFRS1- | 8566 |
| | interacting protein | |
| P51812_C229 | RPS6KA3 Ribosomal protein | 8567 |
| | S6 kinase alpha-3 | |
| Q15418_C223 | RPS6KA1 Ribosomal protein | 8568 |
| | S6 kinase alpha-1 | |
| O94886_C791 | TMEM63A Transmembrane | 8569 |
| | protein 63A | |
| Q14005_C975 | IL16 Pro-interleukin-16 | 8570 |
| P13639_C41 | EEF2 Elongation factor 2 | 8571 |
| Q63HN8_C310 | RNF213 E3 ubiquitin-protein | 8572 |
| | ligase RNF213 | |
| Q96QD9_C242 | FYTTD1 UAP56-interacting | 8573 |
| | factor | |
| P07814_C744 | EPRS Bifunctional | 8574 |
| | glutamate/proline--tRNA | |
| | ligase | |
| Q9UKT9_C434 | IKZF3 Zinc finger protein | 8575 |
| | Aiolos | |
| Q8TC07_C197 | TBC1D15 TBC1 domain | 8576 |
| | family member 15 | |
| Q96RU3_C511 | FNBP1 Formin-binding | 8577 |
| | protein 1 | |
| Q9NUP9_C47 | LIN7C Protein lin-7 homolog | 8578 |
| | C | |
| Q9ULP9_C89 | TBC1D24 TBC1 domain | 8579 |
| | family member 24 | |
| H3BQ06_C89 | Uncharacterized protein | 8580 |
| P28702_C340 | RXRB Retinoic acid receptor | 8581 |
| | RXR-beta | |
| Q9UL15_C327 | BAG5 BAG family molecular | 8582 |
| | chaperone regulator 5 | |
| O95817_C179 | BAG3 BAG family molecular | 8583 |
| | chaperone regulator 3 | |
| Q86UT6_C331 | NLRX1 NLR family member | 8584 |
| | X1 | |
| Q96PE3_C854 | INPP4A Type I inositol 3,4- | 8585 |
| | bisphosphate 4-phosphatase | |
| P57764_C268 | GSDMD Gasdermin-D | 8586 |
| Q99873_C109 | PRMT1 Protein arginine N- | 8587 |
| | methyltransferase 1 | |
| P49589_C27 | CARS Cysteine--tRNA ligase, | 8588 |
| | cytoplasmic | |
| P53384_C31 | NUBP1 Cytosolic Fe—S cluster | 8589 |
| | assembly factor NUBP1 | |
| O95644_C228 | NFATC1 Nuclear factor of | 8590 |
| | activated T-cells, cytoplasmic | |
| | 1 | |
| Q9UPN7_C795 | PPP6R1 Serine/threonine- | 8591 |
| | protein phosphatase 6 | |
| | regulatory | |
| Q93008_C673 | USP9X Probable ubiquitin | 8592 |
| | carboxyl-terminal hydrolase | |
| | FAF | |
| O00507_C674 | USP9Y Probable ubiquitin | 8593 |
| | carboxyl-terminal hydrolase | |
| | FAF | |
| Q9UHI6_C577 | DDX20 Probable ATP- | 8594 |
| | dependent RNA helicase | |
| | DDX20 | |
| Q09666_C108 | AHNAK Neuroblast | 8595 |
| | differentiation-associated | |
| | protein AHNA | |
| O43290_C674 | SART1 U4/U6.U5 tri-snRNP- | 8596 |
| | associated protein 1 | |
| Q2NL82_C126 | TSR1 Pre-rRNA-processing | 8597 |
| | protein TSR1 homolog | |
| Q9UPN3_C4825 | MACF1 Microtubule-actin | 8598 |
| | cross-linking factor 1, | |
| | isoforms | |
| Q13588_C161 | GRAP GRB2-related adapter | 8599 |
| | protein | |
| Q9UG22_C227 | GIMAP2 GTPase IMAP | 8600 |
| | family member 2 | |
| I3L420_C73 | LSM14A Protein LSM14 | 8601 |
| | homolog A | |
| Q8ND56_C78 | LSM14A Protein LSM14 | 8602 |
| | homolog A | |
| Q96CM8_C64 | ACSF2 Acyl-CoA synthetase | 8603 |
| | family member 2, | |
| | mitochondrial | |
| Q6Y7W6_C938 | GIGYF2 PERQ amino acid- | 8604 |
| | rich with GYF domain- | |
| | containing protein | |
| Q9HB58_C327 | SP110 Sp110 nuclear body | 8605 |
| | protein | |
| Q9UGP4_C374 | LIMD1 LIM domain- | 8606 |
| | containing protein 1 | |
| P49458_C48 | SRP9 Signal recognition | 8607 |
| | particle 9 kDa protein | |
| O43566_C496 | RGS14 Regulator of G-protein | 8608 |
| | signaling 14 | |
| P21333_C717 | FLNA Filamin-A | 8609 |
| Q9NZB2_C279 | FAM120A Constitutive | 8610 |
| | coactivator of PPAR-gamma- | |
| | like protein 1 | |
| Q96EB1_C218 | ELP4 Elongator complex | 8611 |
| | protein 4 | |
| O43149_C2312 | ZZEF1 Zinc finger ZZ-type | 8612 |
| | and EF-hand domain- | |
| | containing | |
| Q0VD83_C844 | APOBR Apolipoprotein B | 8613 |
| | receptor | |
| Q00537_C127 | CDK17 Cyclin-dependent | 8614 |
| | kinase 17 | |
| Q9UJY4_C429 | GGA2 ADP-ribosylation | 8615 |
| | factor-binding protein GGA2 | |
| Q03519_C641 | TAP2 Antigen peptide | 8616 |
| | transporter 2 | |
| Q9P2X3_C195 | IMPACT Protein IMPACT | 8617 |
| P42166_C684 | TMPO Lamina-associated | 8618 |
| | polypeptide 2, isoform alpha | |
| Q5GLZ8_C392 | HERC4 Probable E3 | 8619 |
| | ubiquitin-protein ligase | |
| | HERC4 | |
| Q9H832_C100 | UBE2Z Ubiquitin-conjugating | 8620 |
| | enzyme E2 Z | |
| P36969_C93 | GPX4 Phospholipid | 8621 |
| | hydroperoxide glutathione | |
| | peroxidase, mitochondrial | |
| Q9NVG8_C36 | TBC1D13 TBC1 domain | 8622 |
| | family member 13 | |
| O75348_C69 | ATP6V1G1 V-type proton | 8623 |
| | ATPase subunit G 1 | |
| Q99575_C705 | POP1 Ribonucleases P/MRP | 8624 |
| | protein subunit POP1 | |
| O75369_C2501 | FLNB Filamin-B | 8625 |
| P50851_C2675 | LRBA Lipopolysaccharide- | 8626 |
| | responsive and beige-like | |
| | anchor protein | |
| Q99575_C706 | POP1 Ribonucleases P/MRP | 8627 |
| | protein subunit POP1 | |
| Q99704_C115 | DOK1 Docking protein 1 | 8628 |
| Q05086_C108 | UBE3A Ubiquitin-protein | 8629 |
| | ligase E3A | |
| Q15052_C553 | ARHGEF6 Rho guanine | 8630 |
| | nucleotide exchange factor 6 | |
| Q9C0B7_C286 | TMCO7 Transmembrane and | 8631 |
| | coiled-coil domain-containing | |
| | protein | |
| Q9Y2X7_C576 | GIT1 ARF GTPase-activating | 8632 |
| | protein GIT1 | |
| Q66PJ3_C349 | ARL6IP4 ADP-ribosylation | 8633 |
| | factor-like protein 6- | |
| | interacting protein 4 | |
| Q99497_C46 | PARK7 Protein DJ-1 | 8634 |
| Q9UIS9_C525 | MBD1 Methyl-CpG-binding | 8635 |
| | domain protein 1 | |
| Q8IWX8_C69 | CHERP Calcium homeostasis | 8636 |
| | endoplasmic reticulum protein | |
| Q00577_C292 | PURA Transcriptional | 8637 |
| | activator protein Pur-alpha | |
| P62249_C25 | RPS16 40S ribosomal protein | 8638 |
| | S16 | |
| Q9UJX3_C131 | ANAPC7 Anaphase- | 8639 |
| | promoting complex subunit 7 | |
| Q5VTL8_C113 | PRPF38B Pre-mRNA-splicing | 8640 |
| | factor 38B | |
| P47756_C206 | CAPZB F-actin-capping | 8641 |
| | protein subunit beta | |
| Q562E7_C198 | WDR81 WD repeat-containing | 8642 |
| | protein 81 | |
| Q8IV50_C106 | LYSMD2 LysM and putative | 8643 |
| | peptidoglycan-binding | |
| | domain-containing 2 | |
| Q96F15_C171 | GIMAP5 GTPase IMAP | 8644 |
| | family member 5 | |
| Q9P253_C22 | VPS18 Vacuolar protein | 8645 |
| | sorting-associated protein 18 | |
| | homolog | |
| Q562E7_C187 | WDR81 WD repeat-containing | 8646 |
| | protein 81 | |
| Q6KC79_C419 | NIPBL Nipped-B-like protein | 8647 |
| Q9BSJ8_C635 | ESYT1 Extended | 8648 |
| | synaptotagmin-1 | |
| Q14980_C160 | NUMA1 Nuclear mitotic | 8649 |
| | apparatus protein 1 | |
| O00571_C128 | DDX3X ATP-dependent RNA | 8650 |
| | helicase DDX3X | |
| Q96FZ5_C12 | CMTM7 CKLF-like | 8651 |
| | MARVEL transmembrane | |
| | domain-containing protein | |
| Q8TCU6_C52 | PREX1 Phosphatidylinositol | 8652 |
| | 3,4,5-trisphosphate-dependent | |
| Q9Y6K9_C131 | IKBKG NF-kappa-B essential | 8653 |
| | modulator | |
| O14497_C1968 | ARID1A AT-rich interactive | 8654 |
| | domain-containing protein 1A | |
| Q9NYF8_C688 | BCLAF1 Bcl-2-associated | 8655 |
| | transcription factor 1 | |
| Q96SK2_C301 | TMEM209 Transmembrane | 8656 |
| | protein 209 | |
| Q15424_C519 | SAFB Scaffold attachment | 8657 |
| | factor B1 | |
| P52564_C38 | MAP2K6 Dual specificity | 8658 |
| | mitogen-activated protein | |
| | kinase | |
| P08575_C398 | PTPRC Receptor-type | 8659 |
| | tyrosine-protein phosphatase C | |
| Q9NUW8_C135 | TDP1 Tyrosyl-DNA | 8660 |
| | phosphodiesterase 1 | |
| E9PMF8_C332 | PTPRC Receptor-type | 8661 |
| | tyrosine-protein phosphatase C | |
| Q9Y4W2_C140 | LAS1L Ribosomal biogenesis | 8662 |
| | protein LAS1L | |
| P04843_C477 | RPN1 Dolichyl- | 8663 |
| | diphosphooligosaccharide-- | |
| | protein glycosyltransferase | |
| | subunit 1 | |
| Q9UFC0_C249 | LRWD1 Leucine-rich repeat | 8664 |
| | and WD repeat-containing | |
| | protein | |
| Q9NUW8_C48 | TDP1 Tyrosyl-DNA | 8665 |
| | phosphodiesterase 1 | |
| Q15052_C564 | ARHGEF6 Rho guanine | 8666 |
| | nucleotide exchange factor 6 | |
| Q02543_C22 | RPL18A 60S ribosomal | 8667 |
| | protein L18a | |
| A0FGR8_C181 | ESYT2 Extended | 8668 |
| | synaptotagmin-2 | |
| Q9BTA9_C553 | WAC WW domain-containing | 8669 |
| | adapter protein with coiled- | |
| | coil | |
| Q14980_C961 | NUMA1 Nuclear mitotic | 8670 |
| | apparatus protein 1 | |
| Q9BUH6_C180 | C9orf142 Uncharacterized | 8671 |
| | protein C9orf142 | |
| P43250_C138 | GRK6 G protein-coupled | 8672 |
| | receptor kinase 6 | |
| Q15185_C58 | PTGES3 Prostaglandin E | 8673 |
| | synthase 3 | |
| Q8N4N3_C523 | KLHL36 Kelch-like protein 36 | 8674 |
| Q15669_C165 | RHOH Rho-related GTP- | 8675 |
| | binding protein RhoH | |
| Q9NZT2_C443 | OGFR Opioid growth factor | 8676 |
| | receptor | |
| Q13542_C35 | EIF4EBP2 Eukaryotic | 8677 |
| | translation initiation factor 4E- | |
| | binding protein 2 | |
| Q15669_C108 | RHOH Rho-related GTP- | 8678 |
| | binding protein RhoH | |
| Q92835_C138 | INPP5D Phosphatidylinositol | 8679 |
| | 3,4,5-trisphosphate 5- | |
| | phosphatase | |
| Q6PKG0_C864 | LARP1 La-related protein 1 | 8680 |
| Q9Y679_C391 | AUP1 Ancient ubiquitous | 8681 |
| | protein 1 | |
| P36542_C103 | ATP5C1 ATP synthase | 8682 |
| | subunit gamma, mitochondrial | |
| Q01664_C29 | TFAP4 Transcription factor | 8683 |
| | AP-4 | |
| P38606_C138 | ATP6V1A V-type proton | 8684 |
| | ATPase catalytic subunit A | |
| Q9Y520_C223 | PRRC2C Protein PRRC2C | 8685 |
| Q7Z2Z2_C474 | EFTUD1 Elongation factor Tu | 8686 |
| | GTP-binding domain- | |
| | containing | |
| O15294_C758 | OGT UDP-N- | 8687 |
| | acetylglucosamine--peptide N- | |
| | acetylglucosaminyl transferase | |
| Q96S59_C638 | RANBP9 Ran-binding protein | 8688 |
| | 9 | |
| O00541_C272 | PES1 Pescadillo homolog | 8689 |
| Q15020_C670 | SART3 Squamous cell | 8690 |
| | carcinoma antigen recognized | |
| | by T-cells 3 | |
| Q8NF91_C8703 | SYNE1 Nesprin-1 | 8691 |
| Q14966_C1279 | ZNF638 Zinc finger protein | 8692 |
| | 638 | |
| Q96C19_C172 | EFHD2 EF-hand domain- | 8693 |
| | containing protein D2 | |
| O75688_C172 | PPM1B Protein phosphatase | 8694 |
| | 1B | |
| Q05519_C455 | SRSF11 Serine/arginine-rich | 8695 |
| | splicing factor 11 | |
| P42331_C448 | ARHGAP25 Rho GTPase- | 8696 |
| | activating protein 25 | |
| P57075_C371 | UBASH3A Ubiquitin- | 8697 |
| | associated and SH3 domain- | |
| | containing protein | |
| Q96GX2_C75 | ATXN7L3B Putative ataxin-7- | 8698 |
| | like protein 3B | |
| Q14690_C361 | PDCD11 Protein RRP5 | 8699 |
| | homolog | |
| Q15149_C4494 | PLEC Plectin | 8700 |
| Q9NP31_C34 | SH2D2A SH2 domain- | 8701 |
| | containing protein 2A | |
| P30519_C282 | HMOX2 Heme oxygenase 2 | 8702 |
| P98171_C855 | ARHGAP4 Rho GTPase- | 8703 |
| | activating protein 4 | |
| I3L420_C80 | LSM14A Protein LSM14 | 8704 |
| | homolog A | |
| Q8ND56_C85 | LSM14A Protein LSM14 | 8705 |
| | homolog A | |
| P21580_C590 | TNFAIP3 Tumor necrosis | 8706 |
| | factor alpha-induced protein 3 | |
| O14618_C244 | CCS Copper chaperone for | 8707 |
| | superoxide dismutase | |
| Q06124_C573 | PTPN11 Tyrosine-protein | 8708 |
| | phosphatase non-receptor type | |
| | 11 | |
| P08240_C253 | SRPR Signal recognition | 8709 |
| | particle receptor subunit alpha | |
| O14618_C246 | CCS Copper chaperone for | 8710 |
| | superoxide dismutase | |
| Q9H2M9_C67 | RAB3GAP2 Rab3 GTPase- | 8711 |
| | activating protein non-catalytic | |
| | subunit | |
| Q06124_C567 | PTPN11 Tyrosine-protein | 8712 |
| | phosphatase non-receptor type | |
| | 11 | |
| O43865_C272 | AHCYL1 Putative | 8713 |
| | adenosylhomocysteinase 2 | |
| Q96HN2_C353 | AHCYL2 Putative | 8714 |
| | adenosylhomocysteinase 3 | |
| P41226_C599 | UBA7 Ubiquitin-like | 8715 |
| | modifier-activating enzyme 7 | |
| Q06203_C100 | PPAT | 8716 |
| | Amidophosphoribosyltransferase | |
| Q96F07_C98 | CYFIP2 Cytoplasmic FMR1- | 8717 |
| | interacting protein 2 | |
| O60610_C1227 | DIAPH1 Protein diaphanous | 8718 |
| | homolog 1 | |
| Q9Y266_C188 | NUDC Nuclear migration | 8719 |
| | protein nudC | |
| Q7L576_C98 | CYFIP1 Cytoplasmic FMR1- | 8720 |
| | interacting protein 1 | |
| Q9H5N1_C422 | RABEP2 Rab GTPase-binding | 8721 |
| | effector protein 2 | |
| Q9H5N1_C423 | RABEP2 Rab GTPase-binding | 8722 |
| | effector protein 2 | |
| Q13501_C289 | SQSTM1 Sequestosome-1 | 8723 |
| Q02446_C55 | SP4 Transcription factor Sp4 | 8724 |
| O43175_C369 | PHGDH D-3- | 8725 |
| | phosphoglycerate | |
| | dehydrogenase | |
| Q93008_C1237 | USP9X Probable ubiquitin | 8726 |
| | carboxyl-terminal hydrolase | |
| | FAF | |
| O00507_C1238 | USP9Y Probable ubiquitin | 8727 |
| | carboxyl-terminal hydrolase | |
| | FAF | |
| Q8TD19_C11 | NEK9 Serine/threonine- | 8728 |
| | protein kinase Nek9 | |
| Q6ZRI6_C367 | C15orf39 Uncharacterized | 8729 |
| | protein C15orf39 | |
| P40763_C718 | STAT3 Signal transducer and | 8730 |
| | activator of transcription 3 | |
| Q15723_C470 | ELF2 ETS-related | 8731 |
| | transcription factor Elf-2 | |
| Q8IWZ3_C615 | ANKHD1 Ankyrin repeat and | 8732 |
| | KH domain-containing protein | |
| | 1 | |
| O60664_C60 | PLIN3 Perilipin-3 | 8733 |
| Q09666_C5502 | AHNAK Neuroblast | 8734 |
| | differentiation-associated | |
| | protein AHNA | |
| Q8IV63_C191 | VRK3 Inactive | 8735 |
| | serine/threonine-protein kinase | |
| | VRK3 | |
| E7EQZ4_C146 | SMN1 Survival motor neuron | 8736 |
| | protein | |
| Q16637_C146 | SMN2 Survival motor neuron | 8737 |
| | protein | |
| Q8IWV7_C1603 | UBR1 E3 ubiquitin-protein | 8738 |
| | ligase UBR1 | |
| O14981_C936 | BTAF1 TATA-binding | 8739 |
| | protein-associated factor 172 | |
| Q07352_C34 | ZEP36L1 Zinc finger protein | 8740 |
| | 36, C3H1 type-like 1 | |
| P25205_C119 | MCM3 DNA replication | 8741 |
| | licensing factor MCM3 | |
| P35579_C1437 | MYH9 Myosin-9 | 8742 |
| Q8NF50_C2091 | DOCK8 Dedicator of | 8743 |
| | cytokinesis protein 8 | |
| P42224_C492 | STAT1 Signal transducer and | 8744 |
| | activator of transcription 1 | |
| O96008_C74 | TOMM40 Mitochondrial | 8745 |
| | import receptor subunit | |
| | TOM40 homolog | |
| O96008_C76 | TOMM40 Mitochondrial | 8746 |
| | import receptor subunit | |
| | TOM40 homolog | |
| P46063_C606 | RECQL ATP-dependent DNA | 8747 |
| | helicase Q1 | |
| Q9NVE7_C537 | PANK4 Pantothenate kinase 4 | 8748 |
| O15371_C195 | E1F3D Eukaryotic translation | 8749 |
| | initiation factor 3 subunit | |
| P45880_C47 | VDAC2 Voltage-dependent | 8750 |
| | anion-selective channel protein | |
| Q8IZ07_C540 | ANKRD13A Ankyrin repeat | 8751 |
| | domain-containing protein | |
| | 13A | |
| P49795_C73 | RGS19 Regulator of G-protein | 8752 |
| | signaling 19 | |
| P49795_C76 | RGS19 Regulator of G-protein | 8753 |
| | signaling 19 | |
| Q69YN2_C288 | CWF19L1 CWF19-like | 8754 |
| | protein 1 | |
| Q53ET0_C515 | CRTC2 CREB-regulated | 8755 |
| | transcription coactivator 2 | |
| P78371_C412 | CCT2 T-complex protein 1 | 8756 |
| | subunit beta | |
| Q99961_C277 | SH3GL1 Endophilin-A2 | 8757 |
| P33241_C36 | LSP1 Lymphocyte-specific | 8758 |
| | protein 1 | |
| Q8NFI3_C113 | ENGASE Cytosolic endo- | 8759 |
| | beta-N-acetylglucosaminidase | |
| Q9BXJ9_C238 | NAA15 N-alpha- | 8760 |
| | acetyltransferase 15, NatA | |
| | auxiliary subunit | |
| Q92570_C301 | NR4A3 Nuclear receptor | 8761 |
| | subfamily 4 group A member | |
| | 3 | |
| I3L097_C251 | Uncharacterized protein | 8762 |
| Q9H6R4_C1034 | NOL6 Nucleolar protein 6 | 8763 |
| O94992_C79 | HEXIM1 Protein HEXIM1 | 8764 |
| Q8N0X7_C504 | SPG20 Spartin | 8765 |
| O94992_C84 | HEXIM1 Protein HEXIM1 | 8766 |
| Q96HE7_C166 | ERO1L ERO1-like protein | 8767 |
| | alpha | |
| Q9GZU8_C187 | FAM192A Protein FAM192A | 8768 |
| Q9UKA4_C1232 | AKAP11 A-kinase anchor | 8769 |
| | protein 11 | |
| P42224_C155 | STAT1 Signal transducer and | 8770 |
| | activator of transcription 1 | |
| P42166_C280 | TMPO Lamina-associated | 8771 |
| | polypeptide 2, isoform alpha | |
| I3L1I5_C129 | Uncharacterized protein | 8772 |
| Q15052_C25 | ARHGEF6 Rho guanine | 8773 |
| | nucleotide exchange factor 6 | |
| O95267_C615 | RASGRP1 RAS guanyl- | 8774 |
| | releasing protein 1 | |
| Q9P258_C428 | RCC2 Protein RCC2 | 8775 |
| Q7L1Q6_C35 | BZW1 Basic leucine zipper | 8776 |
| | and W2 domain-containing | |
| | protein | |
| Q63HN8_C1510 | RNF213 E3 ubiquitin-protein | 8777 |
| | ligase RNF213 | |
| Q9Y2X3_C439 | NOP58 Nucleolar protein 58 | 8778 |
| P53384_C25 | NUBP1 Cytosolic Fe—S cluster | 8779 |
| | assembly factor NUBP1 | |
| O15054_C1205 | KDM6B Lysine-specific | 8780 |
| | demethylase 6B | |
| Q96BY6_C321 | DOCK10 Dedicator of | 8781 |
| | cytokinesis protein 10 | |
| Q96BY6_C319 | DOCK10 Dedicator of | 8782 |
| | cytokinesis protein 10 | |
| P01892_C363 | HLA-A HLA class I histocompatibility | 8783 |
| | antigen, A-2 alpha | |
| P01891_C363 | HLA-A HLA class I histocompatibility | 8784 |
| | antigen, A-68 alpha | |
| P49327_C1448 | FASN Fatty acid synthase | 8785 |
| Q8ND24_C655 | RNF214 RING finger protein | 8786 |
| | 214 | |
| Q9P2E3_C1822 | ZNFX1 NFX1-type zinc | 8787 |
| | finger-containing protein 1 | |
| Q9Y5K6_C540 | CD2AP CD2-associated | 8788 |
| | protein | |
| Q13459_C1279 | MYO9B Unconventional | 8789 |
| | myosin-IXb | |
| O15355_C241 | PPM1G Protein phosphatase | 8790 |
| | 1G | |
| Q8IV53_C174 | DENND1C DENN domain- | 8791 |
| | containing protein 1C | |
| Q6JBY9_C181 | RCSD1 CapZ-interacting | 8792 |
| | protein | |
| Q8IU85_C354 | CAMK1D | 8793 |
| | Calcium/calmodulin- | |
| | dependent protein kinase type | |
| | 1 | |
| O95644_C209 | NFATC1 Nuclear factor of | 8794 |
| | activated T-cells, cytoplasmic | |
| | 1 | |
| Q92576_C885 | PHF3 PHD finger protein 3 | 8795 |
| Q5T440_C170 | IBA57 Putative transferase | 8796 |
| | CAF17, mitochondrial | |
| P30519_C265 | HMOX2 Heme oxygenase 2 | 8797 |
| Q86UP2_C1105 | KTN1 Kinectin | 8798 |
| O15042_C624 | U2SURP U2 snRNP- | 8799 |
| | associated SURP motif- | |
| | containing protein | |
| Q9NVZ3_C133 | NECAP2 Adaptin ear-binding | 8800 |
| | coat-associated protein 2 | |
| P26651_C253 | ZFP36 Tristetraprolin | 8801 |
| Q5TFE4_C119 | NT5DC1 5-nucleotidase | 8802 |
| | domain-containing protein 1 | |
| Q9BRP1_C278 | PDCD2L Programmed cell | 8803 |
| | death protein 2-like | |
| Q9H410_C287 | DSN1 Kinetochore-associated | 8804 |
| | protein DSN1 homolog | |
| P27986_C146 | PIK3R1 Phosphatidylinositol | 8805 |
| | 3-kinase regulatory subunit | |
| | alpha | |
| P10144_C142 | GZMB Granzyme B | 8806 |
| Q9UKF6_C498 | CPSF3 Cleavage and | 8807 |
| | polyadenylation specificity | |
| | factor subunit | |
| P55265_C1224 | ADAR Double-stranded RNA- | 8808 |
| | specific adenosine deaminase | |
| Q9NXV6_C516 | CDKN2AIP CDKN2A- | 8809 |
| | interacting protein | |
| Q7Z401_C1289 | DENND4A C-myc promoter- | 8810 |
| | binding protein | |
| Q9NVT9_C69 | ARMC1 Armadillo repeat- | 8811 |
| | containing protein 1 | |
| Q9GZR7_C832 | DDX24 ATP-dependent RNA | 8812 |
| | helicase DDX24 | |
| Q2TAA2_C137 | IAH1 Isoamyl acetate- | 8813 |
| | hydrolyzing esterase 1 | |
| | homolog | |
| Q2TAA2_C150 | IAH1 Isoamyl acetate- | 8814 |
| | hydrolyzing esterase 1 | |
| | homolog | |
| Q2TAA2_C145 | IAH1 Isoamyl acetate- | 8815 |
| | hydrolyzing esterase 1 | |
| | homolog | |
| Q13315_C384 | ATM Serine-protein kinase | 8816 |
| | ATM | |
| Q9BXJ9_C721 | NAA15 N-alpha- | 8817 |
| | acetyltransferase 15, NatA | |
| | auxiliary subunit | |
| Q8TD19_C623 | NEK9 Serine/threonine- | 8818 |
| | protein kinase Nek9 | |
| P50749_C251 | RASSF2 Ras association | 8819 |
| | domain-containing protein 2 | |
| Q92831_C690 | KAT2B Histone | 8820 |
| | acetyltransferase KAT2B | |
| Q92830_C695 | KAT2A Histone | 8821 |
| | acetyltransferase KAT2A | |
| P11216_C437 | PYGB Glycogen | 8822 |
| | phosphorylase, brain form | |
| P30044_C100 | PRDX5 Peroxiredoxin-5, | 8823 |
| | mitochondrial | |
| Q9Y3T9_C585 | NOC2L Nucleolar complex | 8824 |
| | protein 2 homolog | |
| Q9NVG8_C387 | TBC1D13 TBC1 domain | 8825 |
| | family member 13 | |
| O14933_C102 | UBE2L6 Ubiquitin/ISG15- | 8826 |
| | conjugating enzyme E2 L6 | |
| Q9UNE7_C199 | STUB1 E3 ubiquitin-protein | 8827 |
| | ligase CHIP | |
| Q9UKA4_C1257 | AKAP11 A-kinase anchor | 8828 |
| | protein 11 | |
| Q8TCU6_C37 | PREX1 Phosphatidylinositol | 8829 |
| | 3,4,5-trisphosphate-dependent | |
| P54274_C118 | TERF1 Telomeric repeat- | 8830 |
| | binding factor 1 | |
| P11161_C469 | EGR2 E3 SUMO-protein | 8831 |
| | ligase EGR2 | |
| P11161_C454 | EGR2 E3 SUMO-protein | 8832 |
| | ligase EGR2 | |
| Q8TAQ2_C145 | SMARCC2 SWI/SNF | 8833 |
| | complex subunit SMARCC2 | |
| Q13232_C158 | NME3 Nucleoside | 8834 |
| | diphosphate kinase 3 | |
| Q9NXV6_C178 | CDKN2AIP CDKN2A- | 8835 |
| | interacting protein | |
| P01106_C25 | MYC Myc proto-oncogene | 8836 |
| | protein | |
| Q6L8Q7_C108 | PDE12 2,5-phosphodiesterase | 8837 |
| | 12 | |
| Q6L8Q7_C119 | PDE12 2,5-phosphodiesterase | 8838 |
| | 12 | |
| P45880_C210 | VDAC2 Voltage-dependent | 8839 |
| | anion-selective channel protein | |
| B0V043_C41 | VARS Valyl-tRNA synthetase | 8840 |
| P45880_C227 | VDAC2 Voltage-dependent | 8841 |
| | anion-selective channel protein | |
| Q9BU23_C696 | LMF2 Lipase maturation | 8842 |
| | factor 2 | |
| Q8NDX1_C435 | PSD4 PH and SEC7 domain- | 8843 |
| | containing protein 4 | |
| Q9Y4B6_C784 | VPRBP Protein VPRBP | 8844 |
| Q8IY21_C1051 | DDX60 Probable ATP- | 8845 |
| | dependent RNA helicase | |
| | DDX60 | |
| O94829_C217 | IPO13 Importin-13 | 8846 |
| Q92835_C1088 | INPP5D Phosphatidylinositol | 8847 |
| | 3,4,5-trisphosphate 5- | |
| | phosphatase | |
| P01106_C70 | MYC Myc proto-oncogene | 8848 |
| | protein | |
| Q99829_C53 | CPNE1 Copine-1 | 8849 |
| Q9Y2S2_C125 | CRYL1 Lambda-crystallin | 8850 |
| | homolog | |
| Q14289_C562 | PTK2B Protein-tyrosine | 8851 |
| | kinase 2-beta | |
| P41240_C290 | CSK Tyrosine-protein kinase | 8852 |
| | CSK | |
| Q01826_C529 | SATB1 DNA-binding protein | 8853 |
| | SATB1 | |
| Q9UPW6_C518 | SATB2 DNA-binding protein | 8854 |
| | SATB2 | |
| Q9GZT9_C127 | EGLN1 Egl nine homolog 1 | 8855 |
| Q99590_C553 | SCAF11 Protein SCAF11 | 8856 |
| Q13077_C37 | TRAF1 TNF receptor- | 8857 |
| | associated factor 1 | |
| Q13077_C38 | TRAF1 TNF receptor- | 8858 |
| | associated factor 1 | |
| O95352_C524 | ATG7 Ubiquitin-like modifier- | 8859 |
| | activating enzyme ATG7 | |
| Q63HN8_C62 | RNF213 E3 ubiquitin-protein | 8860 |
| | ligase RNF213 | |
| Q63HN8_C78 | RNF213 E3 ubiquitin-protein | 8861 |
| | ligase RNF213 | |
| E9PPU0_C1888 | EPPK1 Epiplakin | 8862 |
| Q63HN8_C4258 | RNF213 E3 ubiquitin-protein | 8863 |
| | ligase RNF213 | |
| Q9UKX7_C151 | NUP50 Nuclear pore complex | 8864 |
| | protein Nup50 | |
| P45880_C76 | VDAC2 Voltage-dependent | 8865 |
| | anion-selective channel protein | |
| Q6NYC8_C397 | PPP1R18 Phostensin | 8866 |
| Q6NYC8_C396 | PPP1R18 Phostensin | 8867 |
| Q9ULV4_C420 | CORO1C Coronin-1C | 8868 |
| Q96I15_C22 | SCLY Selenocysteine lyase | 8869 |
| Q8WXH0_C553 | SYNE2 Nesprin-2 | 8870 |
| Q9Y277_C36 | VDAC3 Voltage-dependent | 8871 |
| | anion-selective channel protein | |
| P31153_C104 | MAT2A S- | 8872 |
| | adenosylmethionine synthase | |
| | isoform type-2 | |
| Q14005_C1004 | IL16 Pro-interleukin-16 | 8873 |
| Q16548_C19 | BCL2A1 Bcl-2-related protein | 8874 |
| | A1 | |
| P18583_C92 | SON Protein SON | 8875 |
| P31153_C56 | MAT2A S- | 8876 |
| | adenosylmethionine synthase | |
| | isoform type-2 | |
| Q04759_C14 | PRKCQ Protein kinase C theta | 8877 |
| | type | |
| Q7Z4W1_C244 | DCXR L-xylulose reductase | 8878 |
| Q9Y6C9_C296 | MTCH2 Mitochondrial carrier | 8879 |
| | homolog 2 | |
| Q86YS7_C993 | KIAA0528 Uncharacterized | 8880 |
| | protein KIAA0528 | |
| Q13045_C46 | FLII Protein flightless-1 | 8881 |
| | homolog | |
| Q9UL40_C68 | ZNF346 Zinc finger protein | 8882 |
| | 346 | |
| Q5T4S7_C2554 | UBR4 E3 ubiquitin-protein | 8883 |
| | ligase UBR4 | |
| Q8N0Z8_C292 | PUSL1 tRNA pseudouridine | 8884 |
| | synthase-like 1 | |
| P46109_C249 | CRKL Crk-like protein | 8885 |
| Q96CW5_C194 | TUBGCP3 Gamma-tubulin | 8886 |
| | complex component 3 | |
| P54136_C32 | RARS Arginine--tRNA ligase, | 8887 |
| | cytoplasmic | |
| Q15005_C17 | SPCS2 Signal peptidase | 8888 |
| | complex subunit 2 | |
| Q15306_C194 | IRF4 Interferon regulatory | 8889 |
| | factor 4 | |
| Q96GW9_C425 | MARS2 Methionine--tRNA | 8890 |
| | ligase, mitochondrial | |
| Q02556_C306 | IRF8 Interferon regulatory | 8891 |
| | factor 8 | |
| Q9Y4W2_C456 | LAS1L Ribosomal biogenesis | 8892 |
| | protein LAS1L | |
| Q8TB24_C942 | RIN3 Ras and Rab interactor 3 | 8893 |
| Q96Q11_C373 | TRNT1 CCA tRNA | 8894 |
| | nucleotidyltransferase 1, | |
| | mitochondrial | |
| O00170_C122 | AIP AH receptor-interacting | 8895 |
| | protein | |
| Q7Z2W4_C645 | ZC3HAV1 Zinc finger CCCH- | 8896 |
| | type antiviral protein 1 | |
| O95081_C39 | AGFG2 Arf-GAP domain and | 8897 |
| | FG repeat-containing protein 2 | |
| P11216_C326 | PYGB Glycogen | 8898 |
| | phosphorylase, brain form | |
| Q16548_C55 | BCL2A1 Bcl-2-related protein | 8899 |
| | A1 | |
| Q7Z6Z7_C3372 | HUWE1 E3 ubiquitin-protein | 8900 |
| | ligase HUWE1 | |
| A6NDG6_C297 | PGP Phosphoglycolate | 8901 |
| | phosphatase | |
| O95336_C32 | PGLS 6- | 8902 |
| | phosphogluconolactonase | |
| O14933_C98 | UBE2L6 Ubiquitin/ISG15- | 8903 |
| | conjugating enzyme E2 L6 | |
| P49588_C773 | AARS Alanine--tRNA ligase, | 8904 |
| | cytoplasmic | |
| Q9Y277_C65 | VDAC3 Voltage-dependent | 8905 |
| | anion-selective channel protein | |
| Q6IA69_C428 | NADSYN1 Glutamine- | 8906 |
| | dependent NAD(+) synthetase | |
| Q9Y2W6_C109 | TDRKH Tudor and KH | 8907 |
| | domain-containing protein | |
| Q9NRW3_C130 | APOBEC3C Probable DNA | 8908 |
| | dC- dU-editing enzyme | |
| | APOBEC-3C | |
| P00813_C75 | ADA Adenosine deaminase | 8909 |
| O14920_C464 | IKBKB Inhibitor of nuclear | 8910 |
| | factor kappa-B kinase subunit | |
| Q9NWZ3_C13 | IRAK4 Interleukin-1 receptor- | 8911 |
| | associated kinase 4 | |
| Q13422_C394 | IKZF1 DNA-binding protein | 8912 |
| | Ikaros | |
| Q04759_C17 | PRKCQ Protein kinase C theta | 8913 |
| | type | |
| Q14005_C1011 | IL16 Pro-interleukin-16 | 8914 |
| O75694_C1344 | NUP155 Nuclear pore | 8915 |
| | complex protein Nup155 | |
| Q9BU76_C58 | MMTAG2 Multiple myeloma | 8916 |
| | tumor-associated protein 2 | |
| Q6FI81_C251 | CIAPIN1 Anamorsin | 8917 |
| P54136_C34 | RARS Arginine--tRNA ligase, | 8918 |
| | cytoplasmic | |
| Q3ZCM7_C303 | TUBB8 Tubulin beta-8 chain | 8919 |
| Q3ZCM7_C354 | TUBB8 Tubulin beta-8 chain | 8920 |
| Q13163_C300 | MAP2K5 Dual specificity | 8921 |
| | mitogen-activated protein | |
| | kinase | |
| Q8IZL8_C237 | PELP1 Proline-, glutamic | 8922 |
| | acid- and leucine-rich protein | |
| A0AVT1_C546 | UBA6 Ubiquitin-like | 8923 |
| | modifier-activating enzyme 6 | |
| Q7L2J0_C522 | MEPCE 7SK snRNA | 8924 |
| | methylphosphate capping | |
| | enzyme | |
| Q14657_C23 | LAGE3 L antigen family | 8925 |
| | member 3 | |
| B0I1T2_C979 | MYO1G Unconventional | 8926 |
| | myosin-Ig | |
| P36969_C134 | GPX4 Phospholipid | 8927 |
| | hydroperoxide glutathione | |
| | peroxidase, mitochondrial | |
| Q14149_C15 | MORC3 MORC family CW- | 8928 |
| | type zinc finger protein 3 | |
| P82932_C105 | MRPS6 28S ribosomal protein | 8929 |
| | S6, mitochondrial | |
| Q16666_C637 | IFI16 Gamma-interferon- | 8930 |
| | inducible protein 16 | |
| Q10567_C866 | AP1B1 AP-1 complex subunit | 8931 |
| | beta-1 | |
| Q709C8_C2159 | VPS13C Vacuolar protein | 8932 |
| | sorting-associated protein 13C | |
| Q7Z6Z7_C3375 | HUWE1 E3 ubiquitin-protein | 8933 |
| | ligase HUWE1 | |
| Q7Z6Z7_C3385 | HUWE1 E3 ubiquitin-protein | 8934 |
| | ligase HUWE1 | |
| Q9ULA0_C327 | DNPEP Aspartyl | 8935 |
| | aminopeptidase | |
| P29590_C479 | PML Protein PML | 8936 |
| Q9NRG0_C55 | CHRAC1 Chromatin | 8937 |
| | accessibility complex protein 1 | |
| Q14005_C1016 | IL16 Pro-interleukin-16 | 8938 |
| Q8NCF5_C232 | NFATC2IP NFATC2- | 8939 |
| | interacting protein | |
| Q96QT6_C233 | PHF12 PHD finger protein 12 | 8940 |
| Q29RF7_C532 | PDS5A Sister chromatid | 8941 |
| | cohesion protein PDS5 | |
| | homolog A | |
| Q16254_C88 | E2F4 Transcription factor | 8942 |
| | E2F4 | |
| Q53GL7_C434 | PARP10 Poly | 8943 |
| Q93009_C315 | USP7 Ubiquitin carboxyl- | 8944 |
| | terminal hydrolase 7 | |
| O95931_C102 | CBX7 Chromobox protein | 8945 |
| | homolog 7 | |
| O95931_C107 | CBX7 Chromobox protein | 8946 |
| | homolog 7 | |
| Q5VZ89_C850 | DENND4C DENN domain- | 8947 |
| | containing protein 4C | |
| Q8NF50_C939 | DOCK8 Dedicator of | 8948 |
| | cytokinesis protein 8 | |
| Q7Z3B4_C171 | NUP54 Nucleoporin p54 | 8949 |
| Q6FI81_C249 | CIAPIN1 Anamorsin | 8950 |
| Q92888_C595 | ARHGEF1 Rho guanine | 8951 |
| | nucleotide exchange factor 1 | |
| Q8TDG2_C182 | ACTRT1 Actin-related protein | 8952 |
| | T1 | |
| O60216_C78 | RAD21 Double-strand-break | 8953 |
| | repair protein rad21 homolog | |
| Q9UPN6_C102 | SCAF8 Protein SCAF8 | 8954 |
| Q9NVC6_C649 | MED17 Mediator of RNA | 8955 |
| | polymerase II transcription | |
| | subunit 17 | |
| P35579_C816 | MYH9 Myosin-9 | 8956 |
| Q9P0J1_C149 | PDP1 | 8957 |
| O95602_C613 | POLR1A DNA-directed RNA | 8958 |
| | polymerase I subunit RPA1 | |
| Q8IZL8_C536 | PELP1 Proline-, glutamic | 8959 |
| | acid- and leucine-rich protein | |
| P42331_C154 | ARHGAP25 Rho GTPase- | 8960 |
| | activating protein 25 | |
| P78527_C3837 | PRKDC DNA-dependent | 8961 |
| | protein kinase catalytic subunit | |
| P61978_C185 | HNRNPK Heterogeneous | 8962 |
| | nuclear ribonucleoprotein K | |
| P51610_C352 | HCFC1 Host cell factor 1 | 8963 |
| J3QR44_C422 | CDK11B Cyclin-dependent | 8964 |
| | kinase 11B | |
| P21127_C432 | CDK11B Cyclin-dependent | 8965 |
| | kinase 11B | |
| Q01082_C183 | SPTBN1 Spectrin beta chain, | 8966 |
| | non-erythrocytic 1 | |
| P05771_C502 | PRKCB Protein kinase C beta | 8967 |
| | type | |
| Q9NYQ6_C1840 | CELSR1 Cadherin EGF LAG | 8968 |
| | seven-pass G-type receptor 1 | |
| Q8NBU5_C137 | ATAD1 ATPase family AAA | 8969 |
| | domain-containing protein 1 | |
| Q9BXL7_C539 | CARD11 Caspase recruitment | 8970 |
| | domain-containing protein 11 | |
| Q6PCE3_C303 | PGM2L1 Glucose 1,6- | 8971 |
| | bisphosphate synthase | |
| Q9BTE3_C287 | MCMBP Mini-chromosome | 8972 |
| | maintenance complex-binding | |
| | protein | |
| Q9NTM9_C248 | CUTC Copper homeostasis | 8973 |
| | protein cutC homolog | |
| Q9Y4P1_C74 | ATG4B Cysteine protease | 8974 |
| | ATG4B | |
| P21580_C657 | TNFAIP3 Tumor necrosis | 8975 |
| | factor alpha-induced protein 3 | |
| O43149_C2546 | ZZEF1 Zinc finger ZZ-type | 8976 |
| | and EF-hand domain- | |
| | containing 1 | |
| Q8NHV1_C195 | GIMAP7 GTPase IMAP | 8977 |
| | family member 7 | |
| Q14644_C206 | RASA3 Ras GTPase- | 8978 |
| | activating protein 3 | |
| Q99567_C608 | NUP88 Nuclear pore complex | 8979 |
| | protein Nup88 | |
| P08311_C142 | CTSG Cathepsin G | 8980 |
| O60256_C31 | PRPSAP2 Phosphoribosyl | 8981 |
| | pyrophosphate synthase- | |
| | associated protein 2 | |
| O95785_C1429 | WIZ Protein Wiz | 8982 |
| O00148_C74 | DDX39A ATP-dependent | 8983 |
| | RNA helicase DDX39A | |
| O00148_C86 | DDX39A ATP-dependent | 8984 |
| | RNA helicase DDX39A | |
| Q9H4E7_C266 | DEF6 Differentially expressed | 8985 |
| | in FDCP 6 homolog | |
| P31949_C91 | S100A11 Protein S100-A11 | 8986 |
| P18206_C85 | VCL Vinculin | 8987 |
| P12931_C280 | SRC Proto-oncogene tyrosine- | 8988 |
| | protein kinase Src | |
| Q9UK61_C830 | FAM208A Protein FAM208A | 8989 |
| P19838_C925 | NFKB1 Nuclear factor NF- | 8990 |
| | kappa-B p105 subunit | |
| Q15418_C432 | RPS6KA1 Ribosomal protein | 8991 |
| | S6 kinase alpha-1 | |
| P17987_C397 | TCP1 T-complex protein 1 | 8992 |
| | subunit alpha | |
| P30566_C483 | ADSL Adenylosuccinate lyase | 8993 |
| Q96FV9_C49 | THOC1 THO complex subunit | 8994 |
| | 1 | |
| Q9H9Y6_C1061 | POLR1B DNA-directed RNA | 8995 |
| | polymerase I subunit RPA2 | |
| Q9UN37_C403 | VPS4A Vacuolar protein | 8996 |
| | sorting-associated protein 4A | |
| Q49A26_C303 | GLYR1 Putative | 8997 |
| | oxidoreductase GLYR1 | |
| P49591_C300 | SARS Serine--tRNA ligase, | 8998 |
| | cytoplasmic | |
| P78371_C289 | CCT2 T-complex protein 1 | 8999 |
| | subunit beta | |
| P55786_C265 | NPEPPS Puromycin-sensitive | 9000 |
| | aminopeptidase | |
| Q9Y314_C185 | NOSIP Nitric oxide synthase- | 9001 |
| | interacting protein | |
| P30466_C188 | HLA-B HLA class I histocompatibility | 9002 |
| | antigen, B-18 alpha | |
| P30462_C188 | HLA-B HLA class I histocompatibility | 9003 |
| | antigen, B-14 alpha | |
| Q14980_C80 | NUMA1 Nuclear mitotic | 9004 |
| | apparatus protein 1 | |
| P36954_C52 | POLR2I DNA-directed RNA | 9005 |
| | polymerase II subunit RPB9 | |
| O95639_C41 | CPSF4 Cleavage and | 9006 |
| | polyadenylation specificity | |
| | factor subunit | |
| P14618_C49 | PKM Pyruvate kinase | 9007 |
| | isozymes M1/M2 | |
| Q7L4I2_C382 | RSRC2 Arginine/serine-rich | 9008 |
| | coiled-coil protein 2 | |
| A6NHR9_C1899 | SMCHD1 Structural | 9009 |
| | maintenance of chromosomes | |
| | flexible hinge domain | |
| | containing 1 | |
| Q86U90_C99 | YRDC YrdC domain- | 9010 |
| | containing protein, | |
| | mitochondrial | |
| Q86U90_C95 | YRDC YrdC domain- | 9011 |
| | containing protein, | |
| | mitochondrial | |
| Q14839_C1827 | CHD4 Chromodomain- | 9012 |
| | helicase-DNA-binding protein | |
| | 4 | |
| Q92499_C111 | DDX1 ATP-dependent RNA | 9013 |
| | helicase DDX1 | |
| P49411_C222 | TUFM Elongation factor Tu, | 9014 |
| | mitochondrial | |
| P49411_C290 | TUFM Elongation factor Tu, | 9015 |
| | mitochondrial | |
| Q9Y5U2_C99 | TSSC4 Protein TSSC4 | 9016 |
| P49189_C267 | ALDH9A1 4- | 9017 |
| | trimethylaminobutyraldehyde | |
| | dehydrogenase | |
| O75376_C1274 | NCOR1 Nuclear receptor | 9018 |
| | corepressor 1 | |
| P23743_C645 | DGKA Diacylglycerol kinase | 9019 |
| | alpha | |
| P26368_C429 | U2AF2 Splicing factor U2AF | 9020 |
| | 65 kDa subunit | |
| Q01432_C47 | AMPD3 AMP deaminase 3 | 9021 |
| Q9HBH5_C97 | RDH14 Retinol | 9022 |
| | dehydrogenase 14 | |
| Q13576_C276 | IQGAP2 Ras GTPase- | 9023 |
| | activating-like protein | |
| | IQGAP2 | |
| Q9H5V9_C11 | CXorf56 UPF0428 protein | 9024 |
| | CXorf56 | |
| Q9NYK5_C335 | MRPL39 39S ribosomal | 9025 |
| | protein L39, mitochondrial | |
| Q96JM7_C233 | L3MBTL3 Lethal(3)malignant | 9026 |
| | brain tumor-like protein 3 | |
| P20591_C42 | MX1 Interferon-induced GTP- | 9027 |
| | binding protein Mx1 | |
| Q9NVS2_C186 | MRPS18A 28S ribosomal | 9028 |
| | protein S18a, mitochondrial | |
| O15042_C65 | U2SURP U2 snRNP- | 9029 |
| | associated SURP motif- | |
| | containing protein | |
| Q5H9U9_C1031 | DDX60L Probable ATP- | 9030 |
| | dependent RNA helicase | |
| | DDX60-like | |
| Q9Y4A5_C3535 | TRRAP | 9031 |
| | Transformation/transcription | |
| | domain-associated protein | |
| O60313_C801 | OPA1 Dynamin-like 120 kDa | 9032 |
| | protein, mitochondrial | |
| O60759_C246 | CYTIP Cytohesin-interacting | 9033 |
| | protein | |
| P51531_C1296 | SMARCA2 Probable global | 9034 |
| | transcription activator SNF2L2 | |
| P51532_C1359 | SMARCA4 Transcription | 9035 |
| | activator BRG1 | |
| Q6QNY0_C168 | BLOC1S3 Biogenesis of | 9036 |
| | lysosome-related organelles | |
| | complex | |
| P16615_C447 | ATP2A2 | 9037 |
| | Sarcoplasmic/endoplasmic | |
| | reticulum calcium ATPase | |
| Q6PJI9_C654 | WDR59 WD repeat-containing | 9038 |
| | protein 59 | |
| Q99683_C928 | MAP3K5 Mitogen-activated | 9039 |
| | protein kinase kinase kinase 5 | |
| Q99714_C58 | HSD17B10 3-hydroxyacyl- | 9040 |
| | CoA dehydrogenase type-2 | |
| A6NC98_C1222 | CCDC88B Coiled-coil | 9041 |
| | domain-containing protein | |
| | 88B | |
| P78527_C4045 | PRKDC DNA-dependent | 9042 |
| | protein kinase catalytic subunit | |
| Q9C0B1_C171 | FTO Alpha-ketoglutarate- | 9043 |
| | dependent dioxygenase FTO | |
| Q9Y490_C2161 | TLN1 Talin-1 | 9044 |
| P78527_C3403 | PRKDC DNA-dependent | 9045 |
| | protein kinase catalytic subunit | |
| Q9H668_C8 | OBFC1 CST complex subunit | 9046 |
| | STN1 | |
| Q96FS4_C811 | SIPA1 Signal-induced | 9047 |
| | proliferation-associated | |
| | protein 1 | |
| Q8NI27_C1064 | THOC2 THO complex subunit | 9048 |
| | 2 | |
| O15213_C515 | WDR46 WD repeat-containing | 9049 |
| | protein 46 | |
| P48147_C255 | PREP Prolyl endopeptidase | 9050 |
| B5ME19_C753 | EIF3CL Eukaryotic translation | 9051 |
| | initiation factor 3 subunit | |
| Q15118_C71 | PDK1 | 9052 |
| P12081_C509 | HARS Histidine--tRNA ligase, | 9053 |
| | cytoplasmic | |
| O60684_C253 | KPNA6 Importin subunit | 9054 |
| | alpha-7 | |
| Q9BVP2_C234 | GNL3 Guanine nucleotide- | 9055 |
| | binding protein-like 3 | |
| P09326_C148 | CD48 CD48 antigen | 9056 |
| O75663_C14 | TIPRL TIP41-like protein | 9057 |
| Q9Y4W2_C306 | LAS1L Ribosomal biogenesis | 9058 |
| | protein LAS1L | |
| Q92889_C560 | ERCC4 DNA repair | 9059 |
| | endonuclease XPF | |
| Q9Y4F9_C519 | FAM65B Protein FAM65B | 9060 |
| Q8N8A6_C402 | DDX51 ATP-dependent RNA | 9061 |
| | helicase DDX51 | |
| Q92900_C188 | UPF1 Regulator of nonsense | 9062 |
| | transcripts 1 | |
| Q27J81_C971 | INF2 Inverted formin-2 | 9063 |
| O00422_C26 | SAP18 Histone deacetylase | 9064 |
| | complex subunit SAP18 | |
| Q9BRU9_C207 | UTP23 rRNA-processing | 9065 |
| | protein UTP23 homolog | |
| Q9NV88_C578 | INTS9 Integrator complex | 9066 |
| | subunit 9 | |
| P57740_C78 | NUP107 Nuclear pore | 9067 |
| | complex protein Nup107 | |
| Q15005_C26 | SPCS2 Signal peptidase | 9068 |
| | complex subunit 2 | |
| O75643_C238 | SNRNP200 U5 small nuclear | 9069 |
| | ribonucleoprotein 200 kDa | |
| | helicase | |
| Q92608_C41 | DOCK2 Dedicator of | 9070 |
| | cytokinesis protein 2 | |
| P62913_C72 | RPL11 60S ribosomal protein | 9071 |
| | L11 | |
| Q96FS4_C755 | SIPA1 Signal-induced | 9072 |
| | proliferation-associated | |
| | protein 1 | |
| Q92974_C306 | ARHGEF2 Rho guanine | 9073 |
| | nucleotide exchange factor 2 | |
| Q8TDB6_C175 | DTX3L E3 ubiquitin-protein | 9074 |
| | ligase DTX3L | |
| Q68EM7_C305 | ARHGAP17 Rho GTPase- | 9075 |
| | activating protein 17 | |
| O75131_C54 | CPNE3 Copine-3 | 9076 |
| Q9P107_C895 | GMIP GEM-interacting | 9077 |
| | protein | |
| Q8IYQ7_C324 | THNSL1 Threonine synthase- | 9078 |
| | like 1 | |
| Q05086_C83 | UBE3A Ubiquitin-protein | 9079 |
| | ligase E3A | |
| Q8IWZ3_C181 | ANKHD1 Ankyrin repeat and | 9080 |
| | KH domain-containing protein | |
| | 1 | |
| Q14671_C1179 | PUM1 Pumilio homolog 1 | 9081 |
| Q9BVL2_C252 | NUPL1 Nucleoporin p58/p45 | 9082 |
| Q8NF50_C186 | DOCK8 Dedicator of | 9083 |
| | cytokinesis protein 8 | |
| Q9HB21_C389 | PLEKHA1 Pleckstrin | 9084 |
| | homology domain-containing | |
| | family A member 1 | |
| Q96HC4_C213 | PDLIM5 PDZ and LIM | 9085 |
| | domain protein 5 | |
| Q9UPU5_C1362 | USP24 Ubiquitin carboxyl- | 9086 |
| | terminal hydrolase 24 | |
| Q9Y5T5_C618 | USP16 Ubiquitin carboxyl- | 9087 |
| | terminal hydrolase 16 | |
| Q9Y570_C381 | PPME1 Protein phosphatase | 9088 |
| | methylesterase 1 | |
| Q9NYK5_C133 | MRPL39 39S ribosomal | 9089 |
| | protein L39, mitochondrial | |
| Q96T51_C703 | RUFY1 RUN and FYVE | 9090 |
| | domain-containing protein 1 | |
| Q16643_C613 | DBN1 Drebrin | 9091 |
| Q9Y5B0_C429 | CTDP1 RNA polymerase II | 9092 |
| | subunit A C-terminal domain | |
| | phosphatase | |
| Q8N806_C260 | UBR7 Putative E3 ubiquitin- | 9093 |
| | protein ligase UBR7 | |
| Q96K76_C856 | USP47 Ubiquitin carboxyl- | 9094 |
| | terminal hydrolase 47 | |
| Q9UDY8_C71 | MALT1 Mucosa-associated | 9095 |
| | lymphoid tissue lymphoma | |
| | translocation protein 1 | |
| Q8IY81_C577 | FTSJ3 pre-rRNA processing | 9096 |
| | protein FTSJ3 | |
| P21333_C796 | FLNA Filamin-A | 9097 |
| P11413_C446 | G6PD Glucose-6-phosphate 1- | 9098 |
| | dehydrogenase | |
| P42575_C320 | CASP2 Caspase-2 | 9099 |
| Q04760_C61 | GLO1 Lactoylglutathione | 9100 |
| | lyase | |
| O00562_C259 | PITPNM1 Membrane- | 9101 |
| | associated | |
| | phosphatidylinositol transfer | |
| P49848_C141 | TAF6 Transcription initiation | 9102 |
| | factor TFIID subunit 6 | |
| P08567_C160 | PLEK Pleckstrin | 9103 |
| Q7Z7H8_C180 | MRPL10 39S ribosomal | 9104 |
| | protein L10, mitochondrial | |
| O95786_C268 | DDX58 Probable ATP- | 9105 |
| | dependent RNA helicase | |
| | DDX58 | |
| P17655_C105 | CAPN2 Calpain-2 catalytic | 9106 |
| | subunit | |
| O00541_C361 | PES1 Pescadillo homolog | 9107 |
| Q8WVV9_C405 | HNRPLL Heterogeneous | 9108 |
| | nuclear ribonucleoprotein L- | |
| | like | |
| P45974_C335 | USP5 Ubiquitin carboxyl- | 9109 |
| | terminal hydrolase 5 | |
| P14868_C203 | DARS Aspartate--tRNA | 9110 |
| | ligase, cytoplasmic | |
| Q8WWY3_C247 | PRPF31 U4/U6 small nuclear | 9111 |
| | ribonucleoprotein Prp31 | |
| Q9P258_C280 | RCC2 Protein RCC2 | 9112 |
| P22087_C99 | FBL rRNA 2-O- | 9113 |
| | methyltransferase fibrillarin | |
| Q8N8A2_C408 | ANKRD44 Serine/threonine- | 9114 |
| | protein phosphatase 6 | |
| | regulatory | |
| Q96EB1_C361 | ELP4 Elongator complex | 9115 |
| | protein 4 | |
| P20073_C413 | ANXA7 Annexin A7 | 9116 |
| Q6PGP7_C1162 | TTC37 Tetratricopeptide | 9117 |
| | repeat protein 37 | |
| Q9Y490_C1978 | TLN1 Talin-1 | 9118 |
| Q14204_C1888 | DYNC1H1 Cytoplasmic | 9119 |
| | dynein 1 heavy chain 1 | |
| Q8N8A2_C334 | ANKRD44 Serine/threonine- | 9120 |
| | protein phosphatase 6 | |
| | regulatory | |
| Q96EY4_C162 | TMA16 Translation | 9121 |
| | machinery-associated protein | |
| | 16 | |
| Q9UJX4_C203 | ANAPC5 Anaphase- | 9122 |
| | promoting complex subunit 5 | |
| Q9Y490_C1509 | TLN1 Talin-1 | 9123 |
| P07814_C1480 | EPRS Bifunctional | 9124 |
| | glutamate/proline--tRNA | |
| | ligase | |
| Q14152_C198 | EIF3A Eukaryotic translation | 9125 |
| | initiation factor 3 subunit | |
| P52306_C29 | RAP1GDS1 Rap1 GTPase- | 9126 |
| | GDP dissociation stimulator 1 | |
| Q96RL1_C257 | UIMC1 BRCA1-A complex | 9127 |
| | subunit RAP80 | |
| Q8N4N3_C254 | KLHL36 Kelch-like protein 36 | 9128 |
| Q92616_C55 | GCN1L1 Translational | 9129 |
| | activator GCN1 | |
| Q14966_C652 | ZNF638 Zinc finger protein | 9130 |
| | 638 | |
| P40763_C687 | STAT3 Signal transducer and | 9131 |
| | activator of transcription 3 | |
| P22307_C94 | SCP2 Non-specific lipid- | 9132 |
| | transfer protein | |
| P41250_C466 | GARS Glycine--tRNA ligase | 9133 |
| Q8WXH0_C5315 | SYNE2 Nesprin-2 | 9134 |
| P13639_C567 | EEF2 Elongation factor 2 | 9135 |
| Q99973_C171 | TEP1 Telomerase protein | 9136 |
| | component 1 | |
| Q8N4C6_C1986 | NIN Ninein | 9137 |
| Q8TBC4_C237 | UBA3 NEDD8-activating | 9138 |
| | enzyme E1 catalytic subunit | |
| Q5VWQ0_C318 | RSBN1 Round spermatid | 9139 |
| | basic protein 1 | |
| Q96FS4_C732 | SIPA1 Signal-induced | 9140 |
| | proliferation-associated | |
| | protein 1 | |
| P20700_C198 | LMNB1 Lamin-B1 | 9141 |
| P34949_C289 | MPI Mannose-6-phosphate | 9142 |
| | isomerase | |
| Q5T1M5_C828 | FKBP15 FK506-binding | 9143 |
| | protein 15 | |
| Q13315_C2991 | ATM Serine-protein kinase | 9144 |
| | ATM | |
| Q9UIG0_C953 | BAZ1B Tyrosine-protein | 9145 |
| | kinase BAZ1B | |
| Q9ULX6_C128 | AKAP8L A-kinase anchor | 9146 |
| | protein 8-like | |
| Q92620_C865 | DHX38 Pre-mRNA-splicing | 9147 |
| | factor ATP-dependent RNA | |
| | helicas | |
| Q7L5D6_C160 | GET4 Golgi to ER traffic | 9148 |
| | protein 4 homolog | |
| P34932_C417 | HSPA4 Heat shock 70 kDa | 9149 |
| | protein 4 | |
| Q7KZ85_C1435 | SUPT6H Transcription | 9150 |
| | elongation factor SPT6 | |
| Q9Y2Z2_C315 | MTO1 Protein MTO1 | 9151 |
| | homolog, mitochondrial | |
| P08514_C87 | ITGA2B Integrin alpha-IIb | 9152 |
| O75821_C139 | EIF3G Eukaryotic translation | 9153 |
| | initiation factor 3 subunit | |
| Q9Y265_C141 | RUVBL1 RuvB-like 1 | 9154 |
| Q969M7_C50 | UBE2F NEDD8-conjugating | 9155 |
| | enzyme UBE2F | |
| H3BSR4_C50 | UBE2F Ubiquitin-conjugating | 9156 |
| | enzyme E2F (Putative) | |
| P25098_C120 | ADRBK1 Beta-adrenergic | 9157 |
| | receptor kinase 1 | |
| O60831_C28 | PRAF2 PRA1 family protein 2 | 9158 |
| O00429_C367 | DNM1L Dynamin-1-like | 9159 |
| | protein | |
| Q7Z5K2_C160 | WAPAL Wings apart-like | 9160 |
| | protein homolog | |
| P13807_C699 | GYS1 Glycogen | 9161 |
| Q96CP2_C132 | FLYWCH2 FLYWCH family | 9162 |
| | member 2 | |
| P26651_C250 | ZFP36 Tristetraprolin | 9163 |
| Q92974_C478 | ARHGEF2 Rho guanine | 9164 |
| | nucleotide exchange factor 2 | |
| Q9H4B7_C303 | TUBB1 Tubulin beta-1 chain | 9165 |
| Q9Y5A7_C52 | NUB1 NEDD8 ultimate buster | 9166 |
| | 1 | |
| Q8IZD4_C369 | DCP1B mRNA-decapping | 9167 |
| | enzyme 1B | |
| Q13422_C223 | IKZF1 DNA-binding protein | 9168 |
| | Ikaros | |
| Q15181_C270 | PPA1 Inorganic | 9169 |
| | pyrophosphatase | |
| P49916_C929 | LIG3 DNA ligase 3 | 9170 |
| Q9Y6C9_C49 | MTCH2 Mitochondrial carrier | 9171 |
| | homolog 2 | |
| O15265_C639 | ATXN7 Ataxin-7 | 9172 |
| Q9BQ67_C11 | GRWD1 Glutamate-rich WD | 9173 |
| | repeat-containing protein 1 | |
| Q7Z6Z7_C3658 | HUWE1 E3 ubiquitin-protein | 9174 |
| | ligase HUWE1 | |
| Q9BTE3_C325 | MCMBP Mini-chromosome | 9175 |
| | maintenance complex-binding | |
| | protein | |
| Q92917_C137 | GPKOW G patch domain and | 9176 |
| | KOW motifs-containing | |
| | protein | |
| Q9BSA9_C32 | TMEM175 Transmembrane | 9177 |
| | protein 175 | |
| Q9H9A5_C504 | CNOT10 CCR4-NOT | 9178 |
| | transcription complex subunit | |
| | 10 | |
| P0CB43_C51 | FAM203B Protein FAM203B | 9179 |
| Q9BWU0_C125 | SLC4A1AP Kanadaptin | 9180 |
| Q96FW1_C91 | OTUB1 Ubiquitin thioesterase | 9181 |
| | OTUB1 | |
| Q9UEY8_C73 | ADD3 Gamma-adducin | 9182 |
| Q8N4N3_C308 | KLHL36 Kelch-like protein 36 | 9183 |
| Q9H3M7_C170 | TXNIP Thioredoxin- | 9184 |
| | interacting protein | |
| Q8N9N7_C128 | LRRC57 Leucine-rich repeat- | 9185 |
| | containing protein 57 | |
| I3L1I5_C144 | Uncharacterized protein | 9186 |
| Q7L2J0_C54 | MEPCE 7SK snRNA | 9187 |
| | methylphosphate capping | |
| | enzyme | |
| Q9Y2H0_C800 | DLGAP4 Disks large- | 9188 |
| | associated protein 4 | |
| Q9Y314_C8 | NOSIP Nitric oxide synthase- | 9189 |
| | interacting protein | |
| Q9Y490_C1927 | TLN1 Talin-1 | 9190 |
| Q8N2G8_C502 | GHDC GH3 domain- | 9191 |
| | containing protein | |
| P53992_C78 | SEC24C Protein transport | 9192 |
| | protein Sec24C | |
| P60891_C91 | PRPS1 Ribose-phosphate | 9193 |
| | pyrophosphokinase 1 | |
| P11908_C91 | PRPS2 Ribose-phosphate | 9194 |
| | pyrophosphokinase 2 | |
| P21108_C91 | PRPS1L1 Ribose-phosphate | 9195 |
| | pyrophosphokinase 3 | |
| Q9UPN7_C216 | PPP6R1 Serine/threonine- | 9196 |
| | protein phosphatase 6 | |
| | regulatory | |
| Q63HN8_C3084 | RNF213 E3 ubiquitin-protein | 9197 |
| | ligase RNF213 | |
| P61247_C111 | RPS3A 40S ribosomal protein | 9198 |
| | S3a | |
| P18583_C2070 | SON Protein SON | 9199 |
| P28331_C727 | NDUFS1 NADH-ubiquinone | 9200 |
| | oxidoreductase 75 kDa | |
| | subunit, mitochondrial | |
| P46063_C478 | RECQL ATP-dependent DNA | 9201 |
| | helicase Q1 | |
| Q7L2J0_C429 | MEPCE 7SK snRNA | 9202 |
| | methylphosphate capping | |
| | enzyme | |
| Q9NR45_C287 | NANS Sialic acid synthase | 9203 |
| Q92945_C379 | KHSRP Far upstream element- | 9204 |
| | binding protein 2 | |
| O75934_C132 | BCAS2 Pre-mRNA-splicing | 9205 |
| | factor SPF27 | |
| P42229_C101 | STAT5A Signal transducer | 9206 |
| | and activator of transcription 5 | |
| Q96SI9_C195 | STRBP Spermatid perinuclear | 9207 |
| | RNA-binding protein | |
| P78527_C1629 | PRKDC DNA-dependent | 9208 |
| | protein kinase catalytic subunit | |
| P28715_C12 | ERCC5 DNA repair protein | 9209 |
| | complementing XP-G cells | |
| O60318_C1839 | MCM3AP 80 kDa MCM3- | 9210 |
| | associated protein | |
| P43354_C190 | NR4A2 Nuclear receptor | 9211 |
| | subfamily 4 group A member | |
| | 2 | |
| Q562E7_C215 | WDR81 WD repeat-containing | 9212 |
| | protein 81 | |
| P61247_C139 | RPS3A 40S ribosomal protein | 9213 |
| | S3a | |
| P19174_C646 | PLCG1 1-phosphatidylinositol | 9214 |
| | 4,5-bisphosphate | |
| | phosphodiesterase | |
| Q8IUR7_C283 | ARMC8 Armadillo repeat- | 9215 |
| | containing protein 8 | |
| O75828_C227 | CBR3 Carbonyl reductase | 9216 |
| Q9NR30_C291 | DDX21 Nucleolar RNA | 9217 |
| | helicase 2 | |
| Q9HCK8_C1655 | CHD8 Chromodomain- | 9218 |
| | helicase-DNA-binding protein | |
| | 8 | |
| Q5T4S7_C3864 | UBR4 E3 ubiquitin-protein | 9219 |
| | ligase UBR4 | |
| Q9H869_C290 | YY1AP1 YY1-associated | 9220 |
| | protein 1 | |
| Q3T8J9_C813 | GON4L GON-4-like protein | 9221 |
| P11161_C234 | EGR2 E3 SUMO-protein | 9222 |
| | ligase EGR2 | |
| P50579_C436 | METAP2 Methionine | 9223 |
| | aminopeptidase 2 | |
| P42229_C126 | STAT5A Signal transducer | 9224 |
| | and activator of transcription 5 | |
| Q15545_C72 | TAF7 Transcription initiation | 9225 |
| | factor TFIID subunit 7 | |
| Q9UM19_C187 | HPCAL4 Hippocalcin-like | 9226 |
| | protein 4 | |
| Q92989_C338 | CLP1 Polyribonucleotide 5- | 9227 |
| | hydroxyl-kinase Clp1 | |
| Q5VV42_C138 | CDKAL1 | 9228 |
| | Threonylcarbamoyladenosine | |
| | tRNA methylthiotransferase | |
| Q9BVQ7_C580 | SPATA5L1 Spermatogenesis- | 9229 |
| | associated protein 5-like | |
| | protein | |
| P24928_C451 | POLR2A DNA-directed RNA | 9230 |
| | polymerase II subunit RPB1 | |
| Q14573_C2668 | ITPR3 Inositol 1,4,5- | 9231 |
| | trisphosphate receptor type 3 | |
| Q9NUU7_C392 | DDX19A ATP-dependent | 9232 |
| | RNA helicase DDX19A | |
| Q9UMR2_C393 | DDX19B ATP-dependent | 9233 |
| | RNA helicase DDX19B | |
| P49368_C398 | CCT3 T-complex protein 1 | 9234 |
| | subunit gamma | |
| P0DJD1_C348 | RGPD2 RANBP2-like and | 9235 |
| | GRIP domain-containing | |
| | protein 2 | |
| Q8TF42_C367 | UBASH3B Ubiquitin- | 9236 |
| | associated and SH3 domain- | |
| | containing protein | |
| Q9BWG4_C81 | SSBP4 Single-stranded DNA- | 9237 |
| | binding protein 4 | |
| P50995_C501 | ANXA11 Annexin A11 | 9238 |
| Q9UPP1_C684 | PHF8 Histone lysine | 9239 |
| | demethylase PHF8 | |
| Q9UQ35_C1036 | SRRM2 Serine/arginine | 9240 |
| | repetitive matrix protein 2 | |
| Q9Y6A5_C749 | TACC3 Transforming acidic | 9241 |
| | coiled-coil-containing protein | |
| P13489_C248 | RNH1 Ribonuclease inhibitor | 9242 |
| Q66K74_C588 | MAP1S Microtubule- | 9243 |
| | associated protein 1S | |
| Q16881_C209 | TXNRD1 Thioredoxin | 9244 |
| | reductase 1, cytoplasmic | |
| H0YBQ0_C258 | TXNRD3 Thioredoxin | 9245 |
| | reductase 3 | |
| Q9NNW7_C86 | TXNRD2 Thioredoxin | 9246 |
| | reductase 2, mitochondrial | |
| P78527_C1499 | PRKDC DNA-dependent | 9247 |
| | protein kinase catalytic subunit | |
| Q9UGR2_C956 | ZC3H7B Zinc finger CCCH | 9248 |
| | domain-containing protein 7B | |
| P49903_C71 | SEPHS1 Selenide, water | 9249 |
| | dikinase 1 | |
| O14976_C87 | GAK Cyclin-G-associated | 9250 |
| | kinase | |
| Q86WB0_C406 | ZC3HC1 Nuclear-interacting | 9251 |
| | partner of ALK | |
| Q9H9A6_C23 | LRRC40 Leucine-rich repeat- | 9252 |
| | containing protein 40 | |
| Q3KQU3_C373 | MAP7D1 MAP7 domain- | 9253 |
| | containing protein 1 | |
| Q9H4L5_C24 | OSBPL3 Oxysterol-binding | 9254 |
| | protein-related protein 3 | |
| P27987_C561 | ITPKB Inositol-trisphosphate | 9255 |
| | 3-kinase B | |
| Q9Y520_C177 | PRRC2C Protein PRRC2C | 9256 |
| P53041_C77 | PPP5C Serine/threonine- | 9257 |
| | protein phosphatase 5 | |
| Q9Y2H0_C726 | DLGAP4 Disks large- | 9258 |
| | associated protein 4 | |
| P21580_C54 | TNFAIP3 Tumor necrosis | 9259 |
| | factor alpha-induced protein 3 | |
| Q15334_C505 | LLGL1 Lethal(2) giant larvae | 9260 |
| | protein homolog 1 | |
| O15111_C406 | CHUK Inhibitor of nuclear | 9261 |
| | factor kappa-B kinase subunit | |
| Q14149_C797 | MORC3 MORC family CW- | 9262 |
| | type zinc finger protein 3 | |
| O00329_C219 | PIK3CD Phosphatidylinositol | 9263 |
| | 4,5-bisphosphate 3-kinase | |
| | catalytic subunit delta isoform | |
| Q9NPD3_C97 | EXOSC4 Exosome complex | 9264 |
| | component RRP41 | |
| P98171_C527 | ARHGAP4 Rho GTPase- | 9265 |
| | activating protein 4 | |
| Q8NFI3_C454 | ENGASE Cytosolic endo- | 9266 |
| | beta-N-acetylglucosaminidase | |
| Q13342_C686 | SP140 Nuclear body protein | 9267 |
| | SP140 | |
| Q9H930_C399 | SP140L Nuclear body protein | 9268 |
| | SP140-like protein | |
| Q9C0B0_C696 | UNK RING finger protein | 9269 |
| | unkempt homolog | |
| Q8TB03_C12 | CXorf38 Uncharacterized | 9270 |
| | protein CXorf38 | |
| P21580_C767 | TNFAIP3 Tumor necrosis | 9271 |
| | factor alpha-induced protein 3 | |
| O94906_C698 | PRPF6 Pre-mRNA-processing | 9272 |
| | factor 6 | |
| P43304_C385 | GPD2 Glycerol-3-phosphate | 9273 |
| | dehydrogenase, mitochondrial | |
| Q9H4E7_C267 | DEF6 Differentially expressed | 9274 |
| | in FDCP 6 homolog | |
| Q8NEM7_C298 | FAM48A Protein FAM48A | 9275 |
| O75367_C286 | H2AFY Core histone macro- | 9276 |
| | H2A.1 | |
| O00148_C197 | DDX39A ATP-dependent | 9277 |
| | RNA helicase DDX39A | |
| P40926_C275 | MDH2 Malate dehydrogenase, | 9278 |
| | mitochondrial | |
| Q6P2E9_C249 | EDC4 Enhancer of mRNA- | 9279 |
| | decapping protein 4 | |
| Q9Y6K9_C54 | IKBKG NF-kappa-B essential | 9280 |
| | modulator | |
| Q8TDB6_C159 | DTX3L E3 ubiquitin-protein | 9281 |
| | ligase DTX3L | |
| Q5SSJ5_C359 | HP1BP3 Heterochromatin | 9282 |
| | protein 1-binding protein 3 | |
| P41252_C87 | IARS Isoleucine--tRNA | 9283 |
| | ligase, cytoplasmic | |
| P28838_C445 | LAP3 Cytosol aminopeptidase | 9284 |
| P62979_C144 | RPS27A Ubiquitin-40S | 9285 |
| | ribosomal protein S27a | |
| Q29RF7_C1084 | PDS5A Sister chromatid | 9286 |
| | cohesion protein PDS5 | |
| | homolog A | |
| Q9UPN7_C437 | PPP6R1 Serine/threonine- | 9287 |
| | protein phosphatase 6 | |
| | regulatory | |
| Q04206_C38 | RELA Transcription factor | 9288 |
| | p65 | |
| Q9BTT0_C87 | ANP32E Acidic leucine-rich | 9289 |
| | nuclear phosphoprotein 32 | |
| | family | |
| Q6ZMZ3_C251 | SYNE3 Nesprin-3 | 9290 |
| Q8N201_C969 | INTS1 Integrator complex | 9291 |
| | subunit 1 | |
| Q7Z591_C1078 | AKNA AT-hook-containing | 9292 |
| | transcription factor | |
| Q9ULT8_C1995 | HECTD1 E3 ubiquitin-protein | 9293 |
| | ligase HECTD1 | |
| Q8IZL8_C239 | PELP1 Proline-, glutamic | 9294 |
| | acid- and leucine-rich protein | |
| Q7Z6I6_C1021 | ARHGAP30 Rho GTPase- | 9295 |
| | activating protein 30 | |
| Q8NDX1_C988 | PSD4 PH and SEC7 domain- | 9296 |
| | containing protein 4 | |
| Q92598_C658 | HSPH1 Heat shock protein | 9297 |
| | 105 kDa | |
| Q15020_C537 | SART3 Squamous cell | 9298 |
| | carcinoma antigen recognized | |
| | by T-cells 3 | |
| Q9UJC3_C432 | HOOK1 Protein Hook | 9299 |
| | homolog 1 | |
| Q9BQG0_C1031 | MYBBP1A Myb-binding | 9300 |
| | protein 1A | |
| O00170_C208 | AIP AH receptor-interacting | 9301 |
| | protein | |
| O60244_C729 | MED14 Mediator of RNA | 9302 |
| | polymerase II transcription | |
| | subunit | |
| Q9NUB1_C422 | ACSS1 Acetyl-coenzyme A | 9303 |
| | synthetase 2-like, | |
| | mitochondrial | |
| O76074_C447 | PDE5A cGMP-specific 3,5- | 9304 |
| | cyclic phosphodiesterase | |
| Q96EY5_C231 | FAM125A Multivesicular | 9305 |
| | body subunit 12A | |
| Q9P2I0_C621 | CPSF2 Cleavage and | 9306 |
| | polyadenylation specificity | |
| | factor subunit | |
| Q9NQC7_C129 | CYLD Ubiquitin carboxyl- | 9307 |
| | terminal hydrolase CYLD | |
| P49903_C31 | SEPHS1 Selenide, water | 9308 |
| | dikinase 1 | |
| P22102_C41 | GART Trifunctional purine | 9309 |
| | biosynthetic protein adenosin | |
| Q13464_C1206 | ROCK1 Rho-associated | 9310 |
| | protein kinase 1 | |
| Q01813_C112 | PFKP 6-phosphofructokinase | 9311 |
| | type C | |
| P16188_C188 | HLA-A HLA class I histocompatibility | 9312 |
| | antigen, A-30 alpha | |
| P18463_C188 | HLA-B HLA class I histocompatibility | 9313 |
| | antigen, B-37 alpha | |
| P30499_C188 | HLA-C HLA class I histocompatibility | 9314 |
| | antigen, Cw-1 alpha | |
| P30492_C188 | HLA-B HLA class I histocompatibility | 9315 |
| | antigen, B-54 alpha | |
| Q29940_C188 | HLA-B HLA class I histocompatibility | 9316 |
| | antigen, B-59 alpha | |
| F8VZB9_C225 | HLA-C HLA class I histocompatibility | 9317 |
| | antigen, Cw-14 alpha | |
| P10321_C188 | HLA-C HLA class I histocompatibility | 9318 |
| | antigen, Cw-7 alpha | |
| Q96Q15_C3401 | SMG1 Serine/threonine- | 9319 |
| | protein kinase SMG1 | |
| P27635_C71 | RPL10 60S ribosomal protein | 9320 |
| | L10 | |
| P49756_C83 | RBM25 RNA-binding protein | 9321 |
| | 25 | |
| Q8NE71_C758 | ABCF1 ATP-binding cassette | 9322 |
| | sub-family F member 1 | |
| O95696_C441 | BRD1 Bromodomain- | 9323 |
| | containing protein 1 | |
| Q9NXR7_C34 | BRE BRCA1-A complex | 9324 |
| | subunit BRE | |
| O00478_C512 | BTN3A3 Butyrophilin | 9325 |
| | subfamily 3 member A3 | |
| O60942_C419 | RNGTT mRNA-capping | 9326 |
| | enzyme | |
| Q16831_C17 | UPP1 Uridine phosphorylase 1 | 9327 |
| Q14142_C20 | TRIM14 Tripartite motif- | 9328 |
| | containing protein 14 | |
| O75367_C276 | H2AFY Core histone macro- | 9329 |
| | H2A.1 | |
| Q15052_C57 | ARHGEF6 Rho guanine | 9330 |
| | nucleotide exchange factor 6 | |
| P35626_C340 | ADRBK2 Beta-adrenergic | 9331 |
| | receptor kinase 2 | |
| P25098_C340 | ADRBK1 Beta-adrenergic | 9332 |
| | receptor kinase 1 | |
| P15428_C182 | HPGD 15- | 9333 |
| | hydroxyprostaglandin | |
| | dehydrogenase | |
| Q96FZ2_C131 | C3orf37 UPF0361 protein | 9334 |
| | C3orf37 | |
| Q9Y2H0_C736 | DLGAP4 Disks large- | 9335 |
| | associated protein 4 | |
| Q8TCU4_C2878 | ALMS1 Alstrom syndrome | 9336 |
| | protein 1 | |
| O95801_C238 | TTC4 Tetratricopeptide repeat | 9337 |
| | protein 4 | |
| O95571_C219 | ETHE1 Protein ETHE1, | 9338 |
| | mitochondrial | |
| Q9H4Z3_C29 | PCIF1 Phosphorylated CTD- | 9339 |
| | interacting factor 1 | |
| P55957_C15 | BID BH3-interacting domain | 9340 |
| | death agonist | |
| P49458_C39 | SRP9 Signal recognition | 9341 |
| | particle 9 kDa protein | |
| Q9NQ88_C161 | TIGAR Fructose-2,6- | 9342 |
| | bisphosphatase TIGAR | |
| P22234_C63 | PAICS Multifunctional protein | 9343 |
| | ADE2 | |
| P35611_C253 | ADD1 Alpha-adducin | 9344 |
| P19474_C463 | TRIM21 E3 ubiquitin-protein | 9345 |
| | ligase TRIM21 | |
| Q12874_C103 | SF3A3 Splicing factor 3A | 9346 |
| | subunit 3 | |
| Q03188_C612 | CENPC1 Centromere protein | 9347 |
| | C 1 | |
| P42166_C518 | TMPO Lamina-associated | 9348 |
| | polypeptide 2, isoform alpha | |
| Q9NY12_C80 | GAR1 H/ACA | 9349 |
| | ribonucleoprotein complex | |
| | subunit 1 | |
| Q10567_C241 | AP1B1 AP-1 complex subunit | 9350 |
| | beta-1 | |
| Q96F86_C499 | EDC3 Enhancer of mRNA- | 9351 |
| | decapping protein 3 | |
| Q86UV5_C986 | USP48 Ubiquitin carboxyl- | 9352 |
| | terminal hydrolase 48 | |
| P49368_C213 | CCT3 T-complex protein 1 | 9353 |
| | subunit gamma | |
| P08559_C91 | PDHA1 Pyruvate | 9354 |
| | dehydrogenase E1 component | |
| | subunit alpha, mitochondrial | |
| Q96SZ5_C18 | ADO 2-aminoethanethiol | 9355 |
| | dioxygenase | |
| Q92974_C715 | ARHGEF2 Rho guanine | 9356 |
| | nucleotide exchange factor 2 | |
| Q92597_C289 | NDRG1 Protein NDRG1 | 9357 |
| Q9BZF2_C466 | OSBPL7 Oxysterol-binding | 9358 |
| | protein-related protein 7 | |
| Q9BQ69_C186 | MACROD1 O-acetyl-ADP- | 9359 |
| | ribose deacetylase MACROD1 | |
| Q14738_C17 | PPP2R5D Serine/threonine- | 9360 |
| | protein phosphatase 2A 56 | |
| | kDa regulatory subunit, delta | |
| | isoform | |
| P55210_C186 | CASP7 Caspase-7 | 9361 |
| P31939_C241 | ATIC Bifunctional purine | 9362 |
| | biosynthesis protein PURH | |
| O00329_C366 | PIK3CD Phosphatidylinositol | 9363 |
| | 4,5-bisphosphate 3-kinase | |
| | catalytic subunit delta isoform | |
| Q9NZT2_C417 | OGFR Opioid growth factor | 9364 |
| | receptor | |
| Q96I25_C302 | RBM17 Splicing factor 45 | 9365 |
| | CPTK | |
| Q13158_C98 | FADD Protein FADD | 9366 |
| P52948_C1312 | NUP98 Nuclear pore complex | 9367 |
| | protein Nup98-Nup96 | |
| Q7Z5K2_C964 | WAPAL Wings apart-like | 9368 |
| | protein homolog | |
| Q8NI37_C57 | PPTC7 Protein phosphatase | 9369 |
| | PTC7 homolog | |
| Q9Y6Y8_C604 | SEC23IP SEC23-interacting | 9370 |
| | protein | |
| Q969E8_C114 | TSR2 Pre-rRNA-processing | 9371 |
| | protein TSR2 homolog | |
| Q9UEW8_C82 | STK39 STE20/SPS1-related | 9372 |
| | proline-alanine-rich protein | |
| | kinase | |
| Q9P032_C87 | NDUFAF4 NADH | 9373 |
| | dehydrogenase | |
| Q7L2J0_C244 | MEPCE 7SK snRNA | 9374 |
| | methylphosphate capping | |
| | enzyme | |
| Q70CQ2_C741 | USP34 Ubiquitin carboxyl- | 9375 |
| | terminal hydrolase 34 | |
| Q08378_C1403 | GOLGA3 Golgin subfamily A | 9376 |
| | member 3 | |
| Q14C86_C741 | GAPVD1 GTPase-activating | 9377 |
| | protein and VPS9 domain- | |
| | containing protein 1 | |
| O00267_C626 | SUPT5H Transcription | 9378 |
| | elongation factor SPT5 | |
| P51159_C188 | RAB27A Ras-related protein | 9379 |
| | Rab-27A | |
| Q9NZ63_C145 | C9orf78 Uncharacterized | 9380 |
| | protein C9orf78 | |
| P15374_C95 | UCHL3 Ubiquitin carboxyl- | 9381 |
| | terminal hydrolase isozyme L3 | |
| Q9H9A5_C633 | CNOT10 CCR4-NOT | 9382 |
| | transcription complex subunit | |
| | 10 | |
| Q99614_C28 | TTC1 Tetratricopeptide repeat | 9383 |
| | protein 1 | |
| Q8ND71_C75 | GIMAP8 GTPase IMAP | 9384 |
| | family member 8 | |
| P11171_C224 | EPB41 Protein 4.1 | 9385 |
| Q9H400_C138 | LIME1 Lck-interacting | 9386 |
| | transmembrane adapter 1 | |
| Q9BVC5_C10 | C2orf49 Ashwin | 9387 |
| Q15007_C270 | WTAP Pre-mRNA-splicing | 9388 |
| | regulator WTAP | |
| P17987_C76 | TCP1 T-complex protein 1 | 9389 |
| | subunit alpha | |
| H7BZ11_C99 | Uncharacterized protein | 9390 |
| Q969Q0_C88 | RPL36AL 60S ribosomal | 9391 |
| | protein L36a-like | |
| Q9NS86_C187 | LANCL2 LanC-like protein 2 | 9392 |
| Q9H7N4_C675 | SCAF1 Splicing factor, | 9393 |
| | arginine/serine-rich 19 | |
| Q9Y3S1_C326 | WNK2 Serine/threonine- | 9394 |
| | protein kinase WNK2 | |
| Q9BYP7_C278 | WNK3 Serine/threonine- | 9395 |
| | protein kinase WNK3 | |
| Q9H4A3_C352 | WNK1 Serine/threonine- | 9396 |
| | protein kinase WNK1 | |
| Q8IYL3_C124 | C1orf174 UPF0688 protein | 9397 |
| | C1orf174 | |
| Q86YS7_C449 | KIAA0528 Uncharacterized | 9398 |
| | protein KIAA0528 | |
| Q86UW7_C1052 | CADPS2 Calcium-dependent | 9399 |
| | secretion activator 2 | |
| Q9UBS0_C348 | RPS6KB2 Ribosomal protein | 9400 |
| | S6 kinase beta-2 | |
| O60232_C152 | SSSCA1 Sjoegren | 9401 |
| | syndrome/scleroderma | |
| | autoantigen 1 | |
| Q9NZT2_C87 | OGFR Opioid growth factor | 9402 |
| | receptor | |
| Q8N3D4_C1364 | EHBP1L1 EH domain-binding | 9403 |
| | protein 1-like protein 1 | |
| Q14980_C1729 | NUMA1 Nuclear mitotic | 9404 |
| | apparatus protein 1 | |
| Q15365_C118 | PCBP1 Poly(rC)-binding | 9405 |
| | protein 1 | |
| Q9UP83_C64 | COG5 Conserved oligomeric | 9406 |
| | Golgi complex subunit 5 | |
| Q5RKV6_C117 | EXOSC6 Exosome complex | 9407 |
| | component MTR3 | |
| Q96T23_C1436 | RSF1 Remodeling and spacing | 9408 |
| | factor 1 | |
| P13727_C147 | PRG2 Bone marrow | 9409 |
| | proteoglycan | |
| Q6NYC8_C276 | PPP1R18 Phostensin | 9410 |
| O60282_C393 | KIF5C Kinesin heavy chain | 9411 |
| | isoform 5C | |
| Q92616_C2015 | GCN1L1 Translational | 9412 |
| | activator GCN1 | |
| O75808_C101 | SOLH Calpain-15 | 9413 |
| Q9UQ35_C1029 | SRRM2 Serine/arginine | 9414 |
| | repetitive matrix protein 2 | |
| Q8IZ73_C246 | RPUSD2 RNA | 9415 |
| | pseudouridylate synthase | |
| | domain-containing protein | |
| Q92985_C394 | IRF7 Interferon regulatory | 9416 |
| | factor 7 | |
| P10914_C274 | IRF1 Interferon regulatory | 9417 |
| | factor 1 | |
| P50395_C202 | GDI2 Rab GDP dissociation | 9418 |
| | inhibitor beta | |
| Q9UK61_C690 | FAM208A Protein FAM208A | 9419 |
| Q9UPN7_C612 | PPP6R1 Serine/threonine- | 9420 |
| | protein phosphatase 6 | |
| | regulatory | |
| P07996_C813 | THBS1 Thrombospondin-1 | 9421 |
| Q96GW9_C428 | MARS2 Methionine--tRNA | 9422 |
| | ligase, mitochondrial | |
| O14920_C716 | IKBKB Inhibitor of nuclear | 9423 |
| | factor kappa-B kinase subunit | |
| Q7Z6I6_C20 | ARHGAP30 Rho GTPase- | 9424 |
| | activating protein 30 | |
| Q9NYV4_C1009 | CDK12 Cyclin-dependent | 9425 |
| | kinase 12 | |
| Q9UG63_C388 | ABCF2 ATP-binding cassette | 9426 |
| | sub-family F member 2 | |
| Q96AE4_C332 | FUBP1 Far upstream element- | 9427 |
| | binding protein 1 | |
| Q8N3P4_C1371 | VPS8 Vacuolar protein | 9428 |
| | sorting-associated protein 8 | |
| | homolog | |
| O15541_C15 | RNF113A RING finger | 9429 |
| | protein 113A | |
| Q9UBN7_C426 | HDAC6 Histone deacetylase 6 | 9430 |
| P41252_C526 | IARS Isoleucine--tRNA | 9431 |
| | ligase, cytoplasmic | |
| O60306_C28 | AQR Intron-binding protein | 9432 |
| | aquarius | |
| Q8NFW8_C432 | CMAS N-acylneuraminate | 9433 |
| | cytidylyltransferase | |
| Q9UHB4_C486 | NDOR1 NADPH-dependent | 9434 |
| | diflavin oxidoreductase 1 | |
| Q5TGY3_C1540 | AHDC1 AT-hook DNA- | 9435 |
| | binding motif-containing | |
| | protein 1 | |
| Q8IW35_C484 | CEP97 Centrosomal protein of | 9436 |
| | 97 kDa | |
| Q8WUA4_C212 | GTF3C2 General transcription | 9437 |
| | factor 3C polypeptide 2 | |
| Q6UUV7_C541 | CRTC3 CREB-regulated | 9438 |
| | transcription coactivator 3 | |
| Q5T4S7_C2449 | UBR4 E3 ubiquitin-protein | 9439 |
| | ligase UBR4 | |
| Q13155_C291 | AIMP2 Aminoacyl tRNA | 9440 |
| | synthase complex-interacting | |
| | multif | |
| Q8TEU7_C1561 | RAPGEF6 Rap guanine | 9441 |
| | nucleotide exchange factor 6 | |
| P49792_C1196 | RANBP2 E3 SUMO-protein | 9442 |
| | ligase RanBP2 | |
| O14617_C574 | AP3D1 AP-3 complex subunit | 9443 |
| | delta-1 | |
| Q6P2E9_C54 | EDC4 Enhancer of mRNA- | 9444 |
| | decapping protein 4 | |
| Q5VZ89_C975 | DENND4C DENN domain- | 9445 |
| | containing protein 4C | |
| P57772_C406 | EEFSEC Selenocysteine- | 9446 |
| | specific elongation factor | |
| P30041_C91 | PRDX6 Peroxiredoxin-6 | 9447 |
| Q9H2G2_C1212 | SLK STE20-like | 9448 |
| | serine/threonine-protein kinase | |
| Q16891_C603 | IMMT Mitochondrial inner | 9449 |
| | membrane protein | |
| P55201_C570 | BRPF1 Peregrin | 9450 |
| P09543_C49 | CNP 2,3-cyclic-nucleotide 3- | 9451 |
| | phosphodiesterase | |
| P53701_C178 | HCCS Cytochrome c-type | 9452 |
| | heme lyase | |
| O75376_C2056 | NCOR1 Nuclear receptor | 9453 |
| | corepressor 1 | |
| O60610_C164 | DIAPH1 Protein diaphanous | 9454 |
| | homolog 1 | |
| Q9ULL5_C112 | PRR12 Proline-rich protein 12 | 9455 |
| Q9UMS4_C230 | PRPF19 Pre-mRNA- | 9456 |
| | processing factor 19 | |
| Q99576_C112 | TSC22D3 TSC22 domain | 9457 |
| | family protein 3 | |
| Q9UFW8_C167 | CGGBP1 CGG triplet repeat- | 9458 |
| | binding protein 1 | |
| P62873_C25 | GNB1 Guanine nucleotide- | 9459 |
| | binding protein | |
| | G(I)/G(S)/G(T) | |
| P04049_C27 | RAF1 RAF proto-oncogene | 9460 |
| | serine/threonine-protein kinase | |
| Q9Y6A5_C426 | TACC3 Transforming acidic | 9461 |
| | coiled-coil-containing protein | |
| Q9Y6A5_C502 | TACC3 Transforming acidic | 9462 |
| | coiled-coil-containing protein | |
| Q9H6K4_C164 | OPA3 Optic atrophy 3 protein | 9463 |
| P25205_C360 | MCM3 DNA replication | 9464 |
| | licensing factor MCM3 | |
| Q9UMS0_C213 | NFU1 NFU1 iron-sulfur | 9465 |
| | cluster scaffold homolog, | |
| | mitochondrial | |
| Q08881_C143 | ITK Tyrosine-protein kinase | 9466 |
| | ITK/TSK | |
| Q14974_C455 | KPNB1 Importin subunit beta- | 9467 |
| | 1 | |
| Q8WVJ2_C14 | NUDCD2 NudC domain- | 9468 |
| | containing protein 2 | |
| O00139_C525 | KIF2A Kinesin-like protein | 9469 |
| | KIF2A | |
| Q96T51_C184 | RUFY1 RUN and FYVE | 9470 |
| | domain-containing protein 1 | |
| P08575_C1178 | PTPRC Receptor-type | 9471 |
| | tyrosine-protein phosphatase C | |
| P30626_C75 | SRI Sorcin | 9472 |
| P63010_C95 | AP2B1 AP-2 complex subunit | 9473 |
| | beta | |
| F5H5P2_C231 | Uncharacterized protein | 9474 |
| P12694_C197 | BCKDHA 2-oxoisovalerate | 9475 |
| | dehydrogenase subunit alpha, | |
| | mitochondrial | |
| Q14C86_C1129 | GAPVD1 GTPase-activating | 9476 |
| | protein and VPS9 domain- | |
| | containing protein 1 | |
| A6NKT7_C349 | RGPD3 RanBP2-like and | 9477 |
| | GRIP domain-containing | |
| | protein 3 | |
| Q9Y3L3_C56 | SH3BP1 SH3 domain-binding | 9478 |
| | protein 1 | |
| Q8IXH7_C293 | TH1L Negative elongation | 9479 |
| | factor C/D | |
| Q53ET0_C675 | CRTC2 CREB-regulated | 9480 |
| | transcription coactivator 2 | |
| Q9BZH6_C363 | WDR11 WD repeat-containing | 9481 |
| | protein 11 | |
| Q9BZH6_C364 | WDR11 WD repeat-containing | 9482 |
| | protein 11 | |
| O95466_C733 | FMNL1 Formin-like protein 1 | 9483 |
| P30876_C1155 | POLR2B DNA-directed RNA | 9484 |
| | polymerase II subunit RPB2 | |
| P57764_C457 | GSDMD Gasdermin-D | 9485 |
| P40925_C251 | MDH1 Malate dehydrogenase, | 9486 |
| | cytoplasmic | |
| Q29RF7_C1116 | PDS5A Sister chromatid | 9487 |
| | cohesion protein PDS5 | |
| | homolog A | |
| P15498_C845 | VAV1 Proto-oncogene vav | 9488 |
| Q8N163_C443 | KIAA1967 DBIRD complex | 9489 |
| | subunit KIAA1967 | |
| Q32P44_C307 | EML3 Echinoderm | 9490 |
| | microtubule-associated | |
| | protein-like 3 | |
| Q8WZ74_C759 | CTTNBP2 Cortactin-binding | 9491 |
| | protein 2 | |
| O60264_C165 | SMARCA5 SWI/SNF-related | 9492 |
| | matrix-associated actin- | |
| | dependent | |
| Q9BY77_C338 | POLDIP3 Polymerase delta- | 9493 |
| | interacting protein 3 | |
| P81877_C82 | SSBP2 Single-stranded DNA- | 9494 |
| | binding protein 2 | |
| O43396_C34 | TXNL1 Thioredoxin-like | 9495 |
| | protein 1 | |
| Q7Z6Z7_C1879 | HUWE1 E3 ubiquitin-protein | 9496 |
| | ligase HUWE1 | |
| Q5VT06_C2716 | CEP350 Centrosome- | 9497 |
| | associated protein 350 | |
| O95466_C939 | FMNL1 Formin-like protein 1 | 9498 |
| Q13045_C576 | FLII Protein flightless-1 | 9499 |
| | homolog | |
| Q8WUW1_C43 | BRK1 Protein BRICK1 | 9500 |
| Q86UP2_C303 | KTN1 Kinectin | 9501 |
| Q9BSQ5_C437 | CCM2 Malcavernin | 9502 |
| O43149_C212 | ZZEF1 Zinc finger ZZ-type | 9503 |
| | and EF-hand domain- | |
| | containing | |
| P19338_C543 | NCL Nucleolin | 9504 |
| P10599_C35 | TXN Thioredoxin | 9505 |
| Q9UNI6_C265 | DUSP12 Dual specificity | 9506 |
| | protein phosphatase 12 | |
| O43143_C774 | DHX15 Putative pre-mRNA- | 9507 |
| | splicing factor ATP-dependent | |
| | RN | |
| P25205_C148 | MCM3 DNA replication | 9508 |
| | licensing factor MCM3 | |
| P48553_C1130 | TRAPPC10 Trafficking | 9509 |
| | protein particle complex | |
| | subunit 10 | |
| Q86XR8_C497 | CEP57 Centrosomal protein of | 9510 |
| | 57 kDa | |
| P27987_C178 | ITPKB Inositol-trisphosphate | 9511 |
| | 3-kinase B | |
| Q86W50_C432 | METTL16 Methyltransferase- | 9512 |
| | like protein 16 | |
| O15042_C320 | U2SURP U2 snRNP- | 9513 |
| | associated SURP motif- | |
| | containing protein | |
| Q8IY17_C409 | PNPLA6 Neuropathy target | 9514 |
| | esterase | |
| Q8TDD1_C586 | DDX54 ATP-dependent RNA | 9515 |
| | helicase DDX54 | |
| Q13905_C474 | RAPGEF1 Rap guanine | 9516 |
| | nucleotide exchange factor 1 | |
| A4D1P6_C246 | WDR91 WD repeat-containing | 9517 |
| | protein 91 | |
| P78371_C535 | CCT2 T-complex protein 1 | 9518 |
| | subunit beta | |
| Q08J23_C502 | NSUN2 tRNA (cytosine(34)- | 9519 |
| | C(5))-methyltransferase | |
| Q8IV53_C781 | DENND1C DENN domain- | 9520 |
| | containing protein 1C | |
| O94776_C261 | MTA2 Metastasis-associated | 9521 |
| | protein MTA2 | |
| E7ENX8_C353 | Uncharacterized protein | 9522 |
| Q09666_C2806 | AHNAK Neuroblast | 9523 |
| | differentiation-associated | |
| | protein AHNA | |
| Q63HN8_C2536 | RNF213 E3 ubiquitin-protein | 9524 |
| | ligase RNF213 | |
| Q96EP0_C59 | RNF31 E3 ubiquitin-protein | 9525 |
| | ligase RNF31 | |
| Q86VP6_C1007 | CAND1 Cullin-associated | 9526 |
| | NEDD8-dissociated protein 1 | |
| Q9H0J9_C474 | PARP12 Polymerase | 9527 |
| Q14966_C259 | ZNF638 Zinc finger protein | 9528 |
| | 638 | |
| Q8WWW0_C225 | RASSF5 Ras association | 9529 |
| | domain-containing protein 5 | |
| Q96K58_C126 | ZNF668 Zinc finger protein | 9530 |
| | 668 | |
| Q12768_C385 | KIAA0196 WASH complex | 9531 |
| | subunit strumpellin | |
| Q96CW5_C168 | TUBGCP3 Gamma-tubulin | 9532 |
| | complex component 3 | |
| Q9BYW2_C1471 | SETD2 Histone-lysine N- | 9533 |
| | methyltransferase SETD2 | |
| Q7Z2W4_C518 | ZC3HAV1 Zinc finger CCCH- | 9534 |
| | type antiviral protein 1 | |
| Q6XQN6_C533 | NAPRT1 Nicotinate | 9535 |
| | phosphoribosyltransferase | |
| Q9Y6K9_C347 | IKBKG NF-kappa-B essential | 9536 |
| | modulator K.ASC*QESAR.I | |
| P11142_C603 | HSPA8 Heat shock cognate 71 | 9537 |
| | kDa protein K.VC*NPIITK.L | |
| I3L2F9_C269 | Uncharacterized protein | 9538 |
| | K.NMMAAC*DPR.H | |
| I3L2F9_C320 | Uncharacterized protein | 9539 |
| | K.TAVC*DIPPR.G | |
| Q96JB2_C349 | COG3 Conserved oligomeric | 9540 |
| | Golgi complex subunit 3 | |
| A2A288_C517 | ZC3H12D Probable | 9541 |
| | ribonuclease ZC3H12D | |
| Q9NR33_C85 | POLE4 DNA polymerase | 9542 |
| | epsilon subunit 4 | |
| Q8IXQ6_C728 | PARP9 Polymerase | 9543 |
| Q13422_C254 | IKZF1 DNA-binding protein | 9544 |
| | Ikaros | |
| Q14151_C361 | SAFB2 Scaffold attachment | 9545 |
| | factor B2 | |
| Q8TDX7_C298 | NEK7 Serine/threonine- | 9546 |
| | protein kinase Nek7 | |
| P61962_C256 | DCAF7 DDB1- and CUL4- | 9547 |
| | associated factor 7 | |
| Q00653_C738 | NFKB2 Nuclear factor NF- | 9548 |
| | kappa-B p100 subunit | |
| O95671_C274 | ASMTL N-acetylserotonin O- | 9549 |
| | methyltransferase-like protein | |
| Q92619_C599 | HMHA1 Minor | 9550 |
| | histocompatibility protein HA- | |
| | 1 | |
| Q9UJV9_C568 | DDX41 Probable ATP- | 9551 |
| | dependent RNA helicase | |
| | DDX41 | |
| C9J4G0_C66 | C17orf49 HCG32827, isoform | 9552 |
| | CRA_d | |
| I3L1I5_C221 | Uncharacterized protein | 9553 |
| Q9Y6I9_C165 | TEX264 Testis-expressed | 9554 |
| | sequence 264 protein | |
| Q66K14_C289 | TBC1D9B TBC1 domain | 9555 |
| | family member 9B | |
| Q16875_C155 | PFKFB3 6-phosphofructo-2- | 9556 |
| | kinase/fructose-2,6- | |
| | bisphosphata | |
| Q9UJX3_C329 | ANAPC7 Anaphase- | 9557 |
| | promoting complex subunit 7 | |
| Q9NR56_C27 | MBNL1 Muscleblind-like | 9558 |
| | protein 1 | |
| Q96L21_C105 | RPL10L 60S ribosomal | 9559 |
| | protein L10-like | |
| P03989_C349 | HLA-B HLA class I histocompatibility | 9560 |
| | antigen, B-27 alpha | |
| Q9Y4W2_C469 | LAS1L Ribosomal biogenesis | 9561 |
| | protein LAS1L | |
| Q9P258_C144 | RCC2 Protein RCC2 | 9562 |
| Q96BX8_C88 | MOB3A MOB kinase | 9563 |
| | activator 3A | |
| Q9BW27_C51 | NUP85 Nuclear pore complex | 9564 |
| | protein Nup85 | |
| Q9H582_C507 | ZNF644 Zinc finger protein | 9565 |
| | 644 | |
| Q9H4A6_C84 | GOLPH3 Golgi | 9566 |
| | phosphoprotein 3 | |
| Q9H4A5_C70 | GOLPH3L Golgi | 9567 |
| | phosphoprotein 3-like | |
| Q96DM3_C333 | C18orf8 Uncharacterized | 9568 |
| | protein C18orf8 | |
| P06239_C378 | LCK Tyrosine-protein kinase | 9569 |
| | Lck | |
| Q95365_C125 | HLA-B HLA class I histocompatibility | 9570 |
| | antigen, B-38 alpha | |
| P30453_C125 | HLA-A HLA class I histocompatibility | 9571 |
| | antigen, A-34 alpha | |
| P30479_C125 | HLA-B HLA class I histocompatibility | 9572 |
| | antigen, B-41 alpha | |
| Q29836_C125 | HLA-B HLA class I histocompatibility | 9573 |
| | antigen, B-67 alpha | |
| P30466_C125 | HLA-B HLA class I histocompatibility | 9574 |
| | antigen, B-18 alpha | |
| P30462_C125 | HLA-B HLA class I histocompatibility | 9575 |
| | antigen, B-14 alpha | |
| P30460_C125 | HLA-B HLA class I histocompatibility | 9576 |
| | antigen, B-8 alpha | |
| P01892_C125 | HLA-A HLA class I histocompatibility | 9577 |
| | antigen, A-2 alpha | |
| Q9H7Z7_C110 | PTGES2 Prostaglandin E | 9578 |
| | synthase 2 | |
| Q8TEU7_C1368 | RAPGEF6 Rap guanine | 9579 |
| | nucleotide exchange factor 6 | |
| Q14CN2_C308 | CLCA4 Calcium-activated | 9580 |
| | chloride channel regulator 4 | |
| Q96FK6_C82 | WDR89 WD repeat-containing | 9581 |
| | protein 89 | |
| Q95365_C188 | HLA-B HLA class I histocompatibility | 9582 |
| | antigen, B-38 alpha | |
| Q29836_C188 | HLA-B HLA class I histocompatibility | 9583 |
| | antigen, B-67 alpha | |
| O14981_C939 | BTAF1 TATA-binding | 9584 |
| | protein-associated factor 172 | |
| Q9NXC5_C799 | MIOS WD repeat-containing | 9585 |
| | protein mio | |
| Q13613_C117 | MTMR1 Myotubularin-related | 9586 |
| | protein 1 | |
| Q8IYL3_C215 | C1orf174 UPF0688 protein | 9587 |
| | C1orf174 | |
| Q14289_C899 | PTK2B Protein-tyrosine | 9588 |
| | kinase 2-beta | |
| P26358_C62 | DNMT1 DNA (cytosine-5)- | 9589 |
| | methyltransferase 1 | |
| Q7RTR2_C561 | NLRC3 Protein NLRC3 | 9590 |
| Q9UKA4_C1488 | AKAP11 A-kinase anchor | 9591 |
| | protein 11 | |
| O76075_C194 | DFFB DNA fragmentation | 9592 |
| | factor subunit beta | |
| O00221_C187 | NFKBIE NF-kappa-B | 9593 |
| | inhibitor epsilon | |
| Q96F46_C703 | IL17RA Interleukin-17 | 9594 |
| | receptor A | |
| P36776_C682 | LONP1 Lon protease | 9595 |
| | homolog, mitochondrial | |
| Q9UGK8_C189 | SERGEF Secretion-regulating | 9596 |
| | guanine nucleotide exchange | |
| | factor | |
| Q9HAV4_C1131 | XPO5 Exportin-5 | 9597 |
| P38646_C366 | HSPA9 Stress-70 protein, | 9598 |
| | mitochondrial | |
| P30084_C213 | ECHS1 Enoyl-CoA hydratase, | 9599 |
| | mitochondrial | |
| Q9Y3P8_C126 | SIT1 Signaling threshold- | 9600 |
| | regulating transmembrane | |
| | adaptor 1 | |
| Q92844_C328 | TANK TRAF family member- | 9601 |
| | associated NF-kappa-B | |
| | activator | |
| Q9NR50_C281 | EIF2B3 Translation initiation | 9602 |
| | factor eIF-2B subunit gamma | |
| P36969_C102 | GPX4 Phospholipid | 9603 |
| | hydroperoxide glutathione | |
| | peroxidase, mitochondrial | |
| P29144_C787 | TPP2 Tripeptidyl-peptidase 2 | 9604 |
| P40121_C282 | CAPG Macrophage-capping | 9605 |
| | protein | |
| Q5JSL3_C592 | DOCK11 Dedicator of | 9606 |
| | cytokinesis protein 11 | |
| P52306_C85 | RAP1GDS1 Rap1 GTPase- | 9607 |
| | GDP dissociation stimulator 1 | |
| P61978_C205 | HNRNPK Heterogeneous | 9608 |
| | nuclear ribonucleoprotein K | |
| P35250_C88 | RFC2 Replication factor C | 9609 |
| | subunit 2 | |
| Q96PY6_C276 | NEK1 Serine/threonine- | 9610 |
| | protein kinase Nek1 | |
| Q9Y5J1_C90 | UTP18 U3 small nucleolar | 9611 |
| | RNA-associated protein 18 | |
| | homolog | |
| O95365_C123 | ZBTB7A Zinc finger and BTB | 9612 |
| | domain-containing protein 7A | |
| Q9ULG1_C1001 | INO80 DNA helicase INO80 | 9613 |
| O95801_C63 | TTC4 Tetratricopeptide repeat | 9614 |
| | protein 4 | |
| P57772_C55 | EEFSEC Selenocysteine- | 9615 |
| | specific elongation factor | |
| Q96FE7_C228 | PIK3IP1 Phosphoinositide-3- | 9616 |
| | kinase-interacting protein 1 | |
| Q92945_C176 | KHSRP Far upstream element- | 9617 |
| | binding protein 2 | |
| P15121_C304 | AKR1B1 Aldose reductase | 9618 |
| Q15149_C3017 | PLEC Plectin | 9619 |
| Q12849_C476 | GRSF1 G-rich sequence factor | 9620 |
| | 1 | |
| P55160_C338 | NCKAP1L Nck-associated | 9621 |
| | protein 1-like | |
| P50990_C430 | CCT8 T-complex protein 1 | 9622 |
| | subunit theta | |
| P53597_C60 | SUCLG1 Succinyl-CoA ligase | 9623 |
| Q9Y277_C229 | VDAC3 Voltage-dependent | 9624 |
| | anion-selective channel protein | |
| P30481_C125 | HLA-B HLA class I histocompatibility | 9625 |
| | antigen, B-44 alpha | |
| P04222_C125 | HLA-C HLA class I histocompatibility | 9626 |
| | antigen, Cw-3 alpha | |
| Q96L91_C162 | EP400 E1A-binding protein | 9627 |
| | p400 | |
| Q9Y4E8_C264 | USP15 Ubiquitin carboxyl- | 9628 |
| | terminal hydrolase 15 | |
| Q8IZH2_C1450 | XRN1 5-3 exoribonuclease 1 | 9629 |
| Q29RF7_C581 | PDS5A Sister chromatid | 9630 |
| | cohesion protein PDS5 | |
| | homolog A | |
| Q3KQU3_C251 | MAP7D1 MAP7 domain- | 9631 |
| | containing protein 1 | |
| O60318_C1864 | MCM3AP 80 kDa MCM3- | 9632 |
| | associated protein | |
| Q9NT62_C182 | ATG3 Ubiquitin-like- | 9633 |
| | conjugating enzyme ATG3 | |
| Q14511_C18 | NEDD9 Enhancer of | 9634 |
| | filamentation 1 | |
| O00139_C406 | KIF2A Kinesin-like protein | 9635 |
| | KIF2A | |
| P09914_C138 | IFIT1 Interferon-induced | 9636 |
| | protein with tetratricopeptide | |
| O60216_C35 | RAD21 Double-strand-break | 9637 |
| | repair protein rad21 homolog | |
| P04222_C345 | HLA-C HLA class I histocompatibility | 9638 |
| | antigen, Cw-3 alpha | |
| P57764_C38 | GSDMD Gasdermin-D | 9639 |
| Q9Y520_C2340 | PRRC2C Protein PRRC2C | 9640 |
| P62913_C150 | RPL11 60S ribosomal protein | 9641 |
| | L11 | |
| Q9Y2Y0_C149 | ARL2BP ADP-ribosylation | 9642 |
| | factor-like protein 2-binding | |
| | protein | |
| Q16514_C143 | TAF12 Transcription initiation | 9643 |
| | factor TFIID subunit 12 | |
| O14526_C571 | FCHO1 FCH domain only | 9644 |
| | protein 1 | |
| Q6F5E8_C1339 | RLTPR Leucine-rich repeat- | 9645 |
| | containing protein 16C | |
| Q9H7N4_C362 | SCAF1 Splicing factor, | 9646 |
| | arginine/serine-rich 19 | |
| Q8WW01_C13 | TSEN15 tRNA-splicing | 9647 |
| | endonuclease subunit Sen15 | |
| O15226_C424 | NKRF NF-kappa-B-repressing | 9648 |
| | factor | |
| P40763_C712 | STAT3 Signal transducer and | 9649 |
| | activator of transcription 3 | |
| Q92608_C1083 | DOCK2 Dedicator of | 9650 |
| | cytokinesis protein 2 | |
| Q8TDZ2_C837 | MICAL1 Protein-methionine | 9651 |
| | sulfoxide oxidase MICAL1 | |
| Q9NRA8_C318 | EIF4ENIF1 Eukaryotic | 9652 |
| | translation initiation factor 4E | |
| | transporter | |
| P29590_C227 | PML Protein PML | 9653 |
| Q9UBU9_C252 | NXF1 Nuclear RNA export | 9654 |
| | factor 1 | |
| P61586_C159 | RHOA Transforming protein | 9655 |
| | RhoA | |

Table 5 illustrates an exemplary listing of cysteine-containing polypeptides. The cysteine residue of interest is denoted with (*).

TABLE 5

| Protein | | Cysteine | | SEQ |
|---|---|---|---|---|
| Identifier | | Residue | | ID |
| (Accession No.) | Protein Name | Number | Sequence | NO: |
| O75874 | Isocitrate | C269 | MSKKISGGSVVEMQGDEMTRIIWELIKEKLIFPYV | 9656 |
| | dehydrogenase | | ELDLHSYDLGIENRDATNDQVTKDAAEAIKKHNV | |
| | 1 (IDH1) | | GVKCATITPDEKRVEEFKLKQMWKSPNGTIRNIL | |
| | | | GGTVFREAIICKNIPRLVSGWVKPIIIGRHAYGDQY | |
| | | | RATDFVVPGPGKVEITYTPSDGTQKVTYLVHNFE | |
| | | | EGGGVAMGMYNQDKSIEDFAHSSFQMALSKGWP | |
| | | | LYLSTKNTILKKYDGRFKDIFQEIYDKQYKSQFEA | |
| | | | QKIWYEHRLIDDMVAQAMKSEGGFIWAC*KNYD | |
| | | | GDVQSDSVAQGYGSLGMMTSVLVCPDGKTVEAE | |
| | | | AAHGTVTRHYRMYQKGQETSTNPIASIFAWTRGL | |
| | | | AHRAKLDNNKELAFFANALEEVSIETIEAGFMTK | |
| | | | DLAACIKGLPNVQRSDYLNTFEFMDKLGENLKIK | |
| | | | LAQAKL | |
| P48735 | Isocitrate | C308 | MAGYLRVVRSLCRASGSRPAWAPAALTAPTSQE | 9657 |
| | dehdrogenase | | QPRRHYADKRIKVAKPVVEMDGDEMTRIIWQFIK | |
| | 2 (IDH2) | | EKLILPHVDIQLKYFDLGLPNRDQTDDQVTIDSAL | |
| | | | ATQKYSVAVKCATITPDEARVEEFKLKKMWKSP | |
| | | | NGTIRNILGGTVFREPIICKNIPRLVPGWTKPITIGR | |
| | | | HAHGDQYKATDEVADRAGTFKMVFTPKDGSGV | |
| | | | KEWEVYNFPAGGVGMGMYNTDESISGFAHSCFQ | |
| | | | YAIQKKWPLYMSTKNTILICAYDGRFKDIFQEIFDK | |
| | | | HYKTDFDKNKCIWYEHRLIDDMVAQVLKSSGGFV | |
| | | | WAC*KNYDGDVQSDILAQGFGSLGLMTSVLVCP | |
| | | | DGKTIEAEAAHGTVTRHYREHQKGRPTSTNPIA | |
| | | | SIFAWTRGLEHRGKLDGNQDLIRFAQMLEKVCVE | |
| | | | TVESGAMTKDLAGCIHGLSNVKLNEHFLNTTDFL | |
| | | | DTIKSNLDRALGRQ | |
| Q14790 | CASP8 | C360 | MDFSRNLYDIGEQLDSEDLASLKFLSLDYIPQRKQ | 9658 |
| | | | EPIKDALMLFQRLQEKRMLEESNLSFLKELLFRIN | |
| | | | RLDLLITYLNTRKEEMERELQTPGRAQISAYRVM | |
| | | | LYQISEEVSRSELRSFKFLLQEEISKCKLDDDMNLL | |
| | | | DIFIEMEKRVILGEGKLDILKRVCAQINKSLLKIIN | |
| | | | DYEEFSKERSSSLEGSPDEFSNGEELCGVMTISD | |
| | | | SPREQDSESQTLDKVYQMKSICPRGYCLIINNHNF | |
| | | | AKAREKVPKLHSIRDRNGTHLDAGALTTTFEELH | |
| | | | FEIKPHDDCTVEQIYEILKIYQLMDHSNMDCFICCI | |
| | | | LSHGDKGITYGTDGQEAPIYELTSQFTGLKCPSLA | |
| | | | GKPKVFFIQAC*QGDNYQKGIPVETDSEEQPYLE | |
| | | | MDLSSPQTRYIPDEADFLLGMATVNNCVSYRNPA | |
| | | | EGTWYIQSLCQSLRERCPRGDDILTILTEVNYEV | |
| | | | SNKDDKKNMGKQMPQPTFTLRKKLVFPSD | |
| Q92851 | CASP10 | C401 | MKSQGQHWYS SSDKNCKVSF REKLLIIDSN | 9659 |
| | | | LGVQDVENLK FLCIGLVPNKKLEKSSSASD | |
| | | | VFEHLLAEDL LSEEDPFFLA ELLYIIRQKK | |
| | | | LLQHLNCTKE EVERLLPTRQ RVSLFRNLLY | |
| | | | ELSEGIDSENLKDMIFLLKD SLPKTEMTSL | |
| | | | SFLAFLEKQGKIDEDNLTCL EDLCKTVVPK | |
| | | | LLRNIEKYKREKAIQIVTPP VDKEAESYQG | |
| | | | EEELVSQTDVKTFLEALPQE SWQNKHAGSN | |
| | | | GNRATNGAPSLVSRGMQGASANTLNSETSTKRA | |
| | | | AVYRMNRNHRGLCVIVNNHSFTSLKDR | |
| | | | QGTHICDAEILSHVFQWLGET VHIHNNVTKV | |
| | | | EMEMVLQKQKCNPAHADGDCFVFCILTHGRFGA | |
| | | | VYSSDEALIPIREIMSHFTALQCPRLAEKPKLFFIQ | |
| | | | AC*QGEEIQPSVSIEADALNPEQAPTSLQDSIPAEA | |
| | | | DFLLGLATVPGYVSFRHVEEGSWYIQSLCNHLKK | |
| | | | LVPRMLKFLEKTMEIRGRKRTVWGAICQISATSLP | |
| | | | TAISAQTPRPPMRRWSSVS | |
| Q99873 | PRMT1 | C109 | MENFVATLANGMSLQPPLEEVSCGQAESSEICPNA | 9660 |
| | | | EDMTSKDYYFDSYAHEGIHEEMLKDEVRTLTYRN | |
| | | | SMFHNRHLFKDKVVLDVGSGTGILCMFAAKAGA | |
| | | | RKVIGIEC*SSISDYAVKIVKANKLDHVVTIIKGKV | |
| | | | EEVELPVEKVDIIISEWMGYCLFYESMLNTVLYAR | |
| | | | DKWLAPDGLIFPDRATLYVTAIEDRQYKDYKIHW | |
| | | | WENVYGFDMSCIICDVAIKEPLVDVVDPKQLVTN | |
| | | | ACLIKEVDIYTVKVEDLTFTSPFCLQVKRNDYVH | |
| | | | ALVAYFNIEFTRCHKRTGFSTSPESPYTHWKQTVF | |
| | | | YMEDYLTVKTGEEIFGTIGMRPNAKNNRDLDFTI | |
| | | | DLDFKGQLCELSCSTDYRMR | |
| Q9NYL2 | MAP3 kinase | C22 | MSSLGASFVQIKFDDLQFFENC*GGGSFGSVYRA | 9661 |
| | MLTK (or | | KWISQDKEVAVKKLLKIEKEAEILSVLSHRNIIQFY | |
| | ZAK) | | GVILEPPNYGIVTEYASLGSLYDYINSNRSEEMDM | |
| | | | DHIMTWATDVAKGMHYLHMEAPVKVIHRDLKS | |
| | | | RNVVIAADGVLKCDFGASRFHNHTTHMSLVGTT | |
| | | | PWMAPEVIQSLPVSETCDTYSYGVVLWEMLTREV | |
| | | | PFKGLEGLQVAWLVVEKNERLTIPSSCPRSFAELL | |
| | | | HQCWEADAKICRPSFKQIISILESMSNDTSLPDKCN | |
| | | | SFLHNKAEWRCEIEATLERLKKLERDISFKEQELK | |
| | | | ERERRLKMWEQKLTEQSNTPLLPSFEIGAWTEDD | |
| | | | VYCWVQQLVRKGDSSAEMSVYASLFICENNITGK | |
| | | | RLLLLEEEDLKDMGIVSKGHIIHFKSAIEKLTHDYI | |
| | | | NLFHFPPLIKDSGGEPEENEEKIVNLELVFGFHLKP | |
| | | | GTGPQDCKWKMYMEMDGDEIAITYIKDVTFNTN | |
| | | | LPDAEILKMTKPPFVMEKWIVGIAKSQTVECTVT | |
| | | | YESDVRTPKSTKHVHSIQWSRTKPQDEVKAVQLA | |
| | | | IQTLFTNSDGNPGSRSDSSADCQWLDTLRMRQIAS | |
| | | | NTSLQRSQSNPILGSPFFSHFDGQDSYAAAVRRPQ | |
| | | | VPIKYQQITPVNQSRSSSPTQYGLTKNFSSLHLNSR | |
| | | | DSGFSSGNTDTSSERGRYSDRSRNKYGRGSISLNS | |
| | | | SPRGRYSGKSQHSTPSRGRYPGKFYRVSQSALNP | |
| | | | HQSPDFKRSPRDLHQPNTIPGMPLHPETDSRASEE | |
| | | | DSKVSEGGWTKVEYRKKPHRPSPAKTNKERARG | |
| | | | DHRGWRNF | |
| P12268 | IMPDH2 | C140, | MADYLISGGTSYVPDDGLTAQQLFNCGDGLTYN | 9662 |
| | | C331 | DFLILPGYIDFTADQVDLTSALTKKITLKTPLVSSP | |
| | | | MDTVTEAGMAIAMALTGGIGFIHHNCTPEFQANE | |
| | | | VRKVICKYEQGFITDPVVLSPKDRVRDVFEAKARH | |
| | | | GFC*GIPITDTGRMGSRLVGIISSRDIDFLKEEEHDC | |
| | | | FLEEIMTKREDLVVAPAGITLKEANEILQRSICKGK | |
| | | | LPIVNEDDELVAHARTDLKICNRDYPLASKDAKICQ | |
| | | | LLCGAAIGTHEDDKYRLDLLAQAGVDVVVLDSS | |
| | | | QGNSIFQINMIKYIKDKYPNLQVIGGNVVTAAQA | |
| | | | KNLIDAGVDALRVGMGSGSIC*ITQEVLACGRPQ | |
| | | | ATAVYKVSEYARRFGVPVIADGGIQNVGHIAKAL | |
| | | | ALGASTVMMGSLLAATTEAPGEYFFSDGIRLKKY | |
| | | | RGMGSLDAMDKHLSSQNRYFSEADKIKVAQGVS | |
| | | | GAVQDKGSIHKIVPYLIAGIQHSCQDIGAKSLTQV | |
| | | | RAMMYSGELKFEKRTSSAQVEGGVHSLHSYEKR | |
| | | | LF | |
| Q9NQ88 | TIGAR | C114, | MARFALTVVRHGETRFNKEICHQGQGVDEPLSET | 9663 |
| | | C161 | GFKQAAAAGIFLNNVKFTHAFSSDLMRTKQTMH | |
| | | | GILERSICFCICDMTVKYDSRLRERKYGVVEGICALS | |
| | | | ELRAMAKAAREEC*PVFTPPGGETLDQVKMRGID | |
| | | | FFEFLCQLILKEADQKEQFSQGSPSNC*LETSLAEI | |
| | | | FPLGKNHSSKVNSDSGIPGLAASVLVVSHGAYMR | |
| | | | SLFDYFLTDLKCSLPATLSRSELMSVTPNTGMSLFI | |
| | | | INFEEGREVICPTVQCICMNLQDHLNGLTETR | |
| Q04759 | PKCO | C14, C17 | MSPFLRIGLSNFDC*GSC*QSCQGEAVNPYCAVLV | 9664 |
| | | | KEYVESENGQMYIQKKPTMYPPWDSTFDAHINK | |
| | | | GRVMQIIVKGKNVDLISETTVELYSLAERCRKNN | |
| | | | GKTEIWLELKPQGRMLMNARYFLEMSDTKDMNE | |
| | | | FETEGFFALHQRRGAIKQAKVHHVKCHEFTATFF | |
| | | | PQPTFCSVCHEFVWGLNKQGYQCRQCNAAIHICK | |
| | | | CIDKVIAKCTGSAINSRETMFHKERFKIDMPHRFK | |
| | | | VYNYKSPTFCEHCGTLLWGLARQGLKCDACGMN | |
| | | | VHHRCQTKVANLCGINQKLMAEALAMIESTQQ | |
| | | | ARCLRDTEQIFREGPVEIGLPCSIKNEARPPCLPTP | |
| | | | GKREPQGISWESPLDEVDKMCHLPEPELNKERPSL | |
| | | | QIKLKTEDFILHKMLGKGSFGKVFLAEFKKTNQFF | |
| | | | AIKALKKDVVLMDDDVECTMVEKRVLSLAWEHP | |
| | | | FLTHMFCTFQTKENLFFVMEYLNGGDLMYHIQSC | |
| | | | HKFDLSRATFYAAEIILGLQFLHSKGIVYRDLKLD | |
| | | | NILLDKDGHIKIADFGMCKENNILGDAKTNTFCGT | |
| | | | PDYIAPEILLGQKYNHSVDWWSFGVLLYEMLIGQ | |
| | | | SPFHGQDEEELFHSIRMDNPFYPRWLEKEAKDLL | |
| | | | VKLFVREPEKRLGVRGDIRQHPLFREINWEELERK | |
| | | | EIDPPFRPKVKSPFDCSNFDKEFLNEKPRLSFADRA | |
| | | | LINSMDQNMFRNFSFMNPGMERLIS | |
| Q96SW2 | Cereblon | C188, | MAGEGDQQDAAHNMGNHLPLLPAESEEEDEMEV | 9665 |
| | | C287 | EDQDSKEAKKPNIINFDTSLPTSHTYLGADMEEFH | |
| | | | GRTLHDDDSCQVIPVLPQVMMILTPGQTLPLQLFH | |
| | | | PQEVSMVRNLIQKDRTFAVLAYSNVQEREAQFGT | |
| | | | TAEIYAYREEQDFGIEIVKVKAIGRQRFKVLELRT | |
| | | | QSDGIQQAKVQILPEC*VLPSTMSAVQLESLNKCQ | |
| | | | IFPSKPVSREDQCSYKWWQKYQICRKFHCANLTS | |
| | | | WPRWLYSLYDAETLMDRIKKQLREWDENLKDDS | |
| | | | LPSNPIDFSYRVAAC*LPIDDVLRIQLLKIGSAIQRL | |
| | | | RCELDIMNKCTSLCCKQCQETEITTKNEIFSLSLCG | |
| | | | PMAAYVNPHGYVHETLTVYKACNLNLIGRPSTEH | |
| | | | SWFPGYAWTVAQCKKASHIGWKFTATICKDMSP | |
| | | | QKFWGLTRSALLPTIPDTEDEISPDKVILCL | |

Table 6A-Table 6E illustrate a list of cysteine containing proteins and potential cysteine site of conjugation separated by protein class. Table 6A illustrates cysteine containing enzymes and potential cysteine conjugation site. Table 6B shows a list of cysteine containing transcription factors and regulators. Table 6C shows an exemplary list of cysteine containing channels, transporters and receptors. Table 6D illustrates an exemplary cysteine containing adapter, scaffolding, and modulator protein. Table 6E provides an exemplary list of uncategorized cysteine containing proteins.

TABLE 6A

| | | Cysteine | |
|---|---|---|---|
| Identifier | Protein Name | Location | Protein Class |
| O14920 | IKBKB Inhibitor of nuclear factor kappa-B kinase subunit | C464 | Enzyme |
| O14933 | UBE2L6 Ubiquitin/ISG15-conjugating enzyme E2 L6 PCTK | C98 | Enzyme |
| O94953 | KDM4B Lysine-specific demethylase 4B | C694 | Enzyme |
| P00813 | ADA Adenosine deaminase | C75 | Enzyme |
| P09211 | GSTP1 Glutathione S-transferase P | C48 | Enzyme |
| P15374 | UCHL3 Ubiquitin carboxyl-terminal hydrolase isozyme L3 | C95 | Enzyme |
| P16455 | MGMT Methylated-DNA--protein-cysteine methyltransferase | C145; C150 | Enzyme |
| P17812 | CTP synthase 1 | C491 | Enzyme |
| P19447 | ERCC3 TFIIH basal transcription factor complex helicase | C342 | Enzyme |
| P21580 | TNFAIP3 Tumor necrosis factor alpha-induced protein 3 | C54 | Enzyme |
| P24752 | ACAT1 Acetyl-CoA acetyltransferase, mitochondrial | C119; C126; | Enzyme |
| | | C196; C413 | |
| P40261 | Nicotinamide N-methyltransferase | C165 | Enzyme |
| P41226 | UBA7 Ubiquitin-like modifier-activating enzyme 7 | C599 | Enzyme |
| P42575 | CASP2 Caspase-2 | C370 | Enzyme |
| P43403 | ZAP70 Tyrosine-protein kinase ZAP-70 | C117 | Enzyme |
| P48735 | IDH2 Isocitrate dehydrogenase | C308 | Enzyme |
| P51617 | IRAK1 Interleukin-1 receptor-associated kinase 1 | C608 | Enzyme |
| P61081 | NEDD8-conjugating enzyme Ubc12 | C47 | Enzyme |
| P61088 | Ubiquitin-conjugating enzyme E2 N | C87 | Enzyme |
| P68036 | UBE2L3 Ubiquitin-conjugating enzyme E2 L3 | C86 | Enzyme |
| Q00535 | CDK5 Cyclin-dependent kinase 5 | C157 | Enzyme |
| Q04759 | PRKCQ Protein kinase C theta type | C14; C17 | Enzyme |
| Q06124 | Tyrosine-protein phosphatase non-receptor type 11 | C573 | Enzyme |
| Q09472 | EP300 Histone acetyltransferase p300 | C1738 | Enzyme |
| Q14790 | CASP8 Caspase-8 | C360 | Enzyme |
| Q15084 | PDIA6 Protein disulfide-isomerase A6 | C55; C58; | Enzyme |
| | | C190; C193 | |
| Q15910 | EZH2 Histone-lysine N-methyltransferase EZH2 | C503 | Enzyme |
| Q16763 | UBE2S Ubiquitin-conjugating enzyme E2 S | C118 | Enzyme |
| Q16822 | PCK2 Phosphoenolpyruvate carboxykinase | C306 | Enzyme |
| Q16875 | 6-phosphofructo-2-kinase/fructose-2,6-bisphosphatase 3 | C155 | Enzyme |
| Q16877 | PFKFB4 6-phosphofructo-2-kinase/fructose-2,6-bisphosphata | C159 | Enzyme |
| Q6L8Q7 | PDE12 2,5-phosphodiesterase 12 | C108 | Enzyme |
| Q70CQ2 | USP34 Ubiquitin carboxyl-terminal hydrolase 34 | C741; C1090 | Enzyme |
| Q86UV5 | USP48 Ubiquitin carboxyl-terminal hydrolase 48 | C39 | Enzyme |
| Q92851 | Caspase-10 | C401 | Enzyme |
| Q93009 | USP7 Ubiquitin carboxyl-terminal hydrolase 7 | C223; C315 | Enzyme |
| Q96FA3 | PELI1 E3 ubiquitin-protein ligase pellino homolog 1 | C282 | Enzyme |
| Q96JH7 | VCPIP1 Deubiquitinating protein VCIP135 | C219 | Enzyme |
| Q96RU2 | USP28 Ubiquitin carboxyl-terminal hydrolase 28 | C171; C733 | Enzyme |
| Q99873 | PRMT1 Protein arginine N-methyltransferase 1 | C109 | Enzyme |
| Q9C0C9 | UBE2O Ubiquitin-conjugating enzyme E2 O | C375 | Enzyme |
| Q9NRW4 | Dual specificity protein phosphatase 22 | C124 | Enzyme |
| Q9NWZ3 | IRAK4 Interleukin-1 receptor-associated kinase 4 | C13 | Enzyme |
| Q9NYL2 | MLTK Mitogen-activated protein kinase kinase kinase MLT | C22 | Enzyme |
| Q9UPT9 | USP22 Ubiquitin carboxyl-terminal hydrolase 22 | C44; C71 | Enzyme |
| Q9Y3Z3 | SAMHD1 SAM domain and HD domain-containing protein 1 | C522 | Enzyme |
| Q9Y4C1 | KDM3A Lysine-specific demethylase 3A | C251 | Enzyme |
| Q9Y5T5 | USP16 Ubiquitin carboxyl-terminal hydrolase 16 | C205 | Enzyme |

TABLE 6B

| | | Cysteine | |
|---|---|---|---|
| Identifier | Protein Name | Location | Protein Class |
| O75362 | ZNF217 Zinc finger protein 217 | C286 | Transcription |
| | | | factors and |
| | | | regulators |
| P04150 | NR3C1 Glucocorticoid receptor | C302; C622 | Transcription |
| | | | factors and |
| | | | regulators |
| P09086 | POU2F2 POU domain, class 2, transcription factor 2 | C346 | Transcription |
| | | | factors and |
| | | | regulators |
| P40763 | STAT3 Signal transducer and activator of transcription 3 | C259 | Transcription |
| | | | factors and |
| | | | regulators |
| P48200 | IREB2 Iron-responsive element-binding protein 2 | C137 | Transcription |
| | | | factors and |
| | | | regulators |
| Q01201 | RELB Transcription factor RelB | C109 | Transcription |
| | | | factors and |
| | | | regulators |
| Q02556 | IRF8 Interferon regulatory factor 8 | C306 | Transcription |
| | | | factors and |
| | | | regulators |
| Q15306 | IRF4 Interferon regulatory factor 4 | C194 | Transcription |
| | | | factors and |
| | | | regulators |
| Q7Z2W4 | ZC3HAV1 Zinc finger CCCH-type antiviral protein 1 | C645 | Transcription |
| | | | factors and |
| | | | regulators |
| Q8TAQ2 | SMARCC2 SWI/SNF complex subunit SMARCC2 | C145 | Transcription |
| | | | factors and |
| | | | regulators |

TABLE 6C

| | | Cysteine | |
|---|---|---|---|
| Identifier | Protein Name | Location | Protein Class |
| P63244 | GNB2L1 Guanine nucleotide-binding protein subunit | C182 | Channels, |
| | beta-2-like 1 | | Transporters, |
| | | | Receptors |
| Q16186 | Proteasomal ubiquitin receptor ADRM1 | C88 | Channels, |
| | | | Transporters, |
| | | | Receptors |
| Q9HB90 | RRAGC Ras-related GTP-binding protein C | C358; C377 | Channels, |
| | | | transporters, |
| | | | and receptors |

TABLE 6D

| | | Cysteine | |
|---|---|---|---|
| Identifier | Protein Name | Location | Protein Class |
| P14598 | NCF1 Neutrophil cytosol factor 1 | C378 | Adapter, |
| | | | scaffolding, |
| | | | modulator proteins |

TABLE 6E

| | | Cysteine | |
|---|---|---|---|
| Identifier | Protein Name | Location | Protein Class |
| O00170 | AIP AH receptor-interacting protein | C122 | Uncategorized |
| O00541 | PES1 Pescadillo homolog | C272; C361 | Uncategorized |
| O00622 | CYR61 Protein CYR61 | C39; C70; | Uncategorized |
| | | C134 | |
| O14980 | XPO1 Exportin-1 | C34; C528; | Uncategorized |
| | | C1070 | |
| P50851 | LRBA Lipopolysaccharide-responsive and beige-like | C1704; C2675 | Uncategorized |
| | anchor protein | | |
| Q96GG9 | DCUN1D1 DCN1-like protein 1 | C115 | Uncategorized |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods within the scope of these claims and their equivalents be covered thereby.

What is claimed is:

1. A method of modulating an immune response in a subject in need thereof, comprising administering to the subject a therapeutically effective amount of a small molecule fragment of Formula (I): 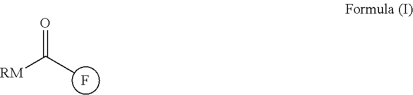
wherein:
RM is a reactive moiety selected from a Michael acceptor moiety, a leaving group moiety, or a moiety capable of forming a covalent bond with the thiol group of a cysteine residue; and
F is a small molecule fragment moiety.

2. The method of claim 1, wherein the small molecule fragment interacts with an endogenous cysteine-containing polypeptide expressed in the subject to form a cysteine-containing polypeptide-small molecule fragment adduct.

3. The method of claim 1 or 2, wherein the small molecule fragment is covalently bond to a cysteine residue of the cysteine-containing polypeptide.

4. The method of claim 2, wherein the cysteine-containing polypeptide-small molecule fragment adduct induces an immune response.

5. The method of claim 2 or 4, wherein the cysteine-containing polypeptide-small molecule fragment adduct induces a humoral immune response or a cell-mediated immune response.

6. The method of claim 2, wherein the cysteine-containing polypeptide-small molecule fragment adduct increases an immune response relative to a control.

7. The method of claim 6, wherein the control is the level of an immune response in the subject prior to administration of the small molecule fragment or the level of an immune response in a subject who has not been exposed to the small molecule fragment.

8. The method of claim 2, wherein the cysteine-containing polypeptide is overexpressed in a disease or condition.

9. The method of claim 2, wherein the cysteine-containing polypeptide comprises one or more mutations, optionally overexpressed in a disease or condition.

10. The method of claim 8 or 9, wherein the disease or condition is cancer.

11. The method of claim 2, wherein the cysteine-containing polypeptide comprises a biologically active cysteine site, optionally located about 10 Å or less to an active-site ligand or residue.

12. The method of claim 2, wherein the cysteine-containing polypeptide comprises a protein illustrated in Tables 1-6.

13. The method of claim 2, wherein the cysteine-containing polypeptide comprises cereblon.

14. The method of claim 2, wherein the cysteine-containing polypeptide is at most 50 amino acid residues in length.

15. The method of claim 14, wherein the cysteine-containing polypeptide comprises an isolated and purified polypeptide comprising at least 80%, 85%, 90%, 95%, 96%, 9%, 98%, or 99% sequence identity to at least seven contiguous amino acids of an amino acid sequence selected from SEQ ID NOs: 1-9655.

16. The method of claim 1, wherein F is a small molecule fragment moiety illustrated in FIG. 1.

17. An isolated and purified antibody or its binding fragment thereof comprising a heavy chain CDR1, CDR2 and CDR3 sequence and a light chain CDR1, CDR2 and CDR3 sequence, wherein the heavy chain and light chain CDRs interact with a cysteine-containing polypeptide that is covalently bond to a small molecule fragment, wherein the small molecule fragment is a small molecule fragment of Formula (I): 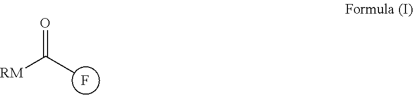
wherein:
RM is a reactive moiety selected from a Michael acceptor moiety, a leaving group moiety, or a moiety capable of forming a covalent bond with the thiol group of a cysteine residue; and
wherein the small molecule fragment is covalently bond to a cysteine residue of the cysteine-containing polypeptide.

18. The isolated and purified antibody or its binding fragment thereof of claim 17, wherein the antibody or its binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof.

19. The isolated and purified antibody or its binding fragment thereof of claim 17, wherein F is a small molecule fragment moiety illustrated in FIG. 1.

20. The isolated and purified antibody or its binding fragment thereof of claim 17, wherein the cysteine-containing polypeptide comprises a protein illustrated in Tables 1-6.

21. The isolated and purified antibody or its binding fragment thereof of claim 17, wherein the cysteine-containing polypeptide comprises cereblon.

22. The isolated and purified antibody or its binding fragment thereof of claim 17, wherein the cysteine-containing polypeptide is at most 50 amino acid residues in length.

23. The isolated and purified antibody or its binding fragment thereof of claim 22, wherein the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQ ID NOs: 1-9655.

24. A vaccine comprising a small molecule fragment of Formula (I): 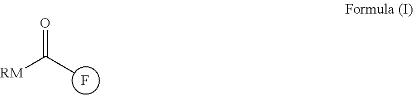
wherein:
RM is a reactive moiety selected from a Michael acceptor moiety, a leaving group moiety, or a moiety capable of forming a covalent bond with the thiol group of a cysteine residue; and
F is a small molecule fragment moiety.

25. The vaccine of claim 24, wherein F is a small molecule fragment moiety illustrated in FIG. 1.

26. A kit comprising an isolated and purified antibody or its binding fragment thereof of claims 17-23, or a vaccine of claims 24-25.

27. A method of modulating cereblon activity, comprising:
contacting a cell expressing cereblon with a small molecule fragment of Formula (I): 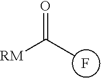
wherein EM is a reactive moiety selected from a Michael acceptor moiety, a leaving group moiety, or a moiety capable of forming a covalent bond with the thiol group of cysteine residue; and F is a small molecule fragment moiety;
wherein the small molecule fragment of Formula (I) covalently binds to residue 187 or residue 288 of cereblon; and
wherein residue positions 187 and 288 correspond to positions 187 and 288 of SEQ ID NO: 9665.

28. The method of claim 27, wherein F is a small molecule fragment moiety illustrated in FIG. 1.

29. The method of claim 27, wherein F optionally comprises a second reactive moiety.

30. The method of claim 27, wherein the cell is a mammalian cell.

31. The method of claim 27, wherein the method is an in vivo method.
